(19) United States
(12) Patent Application Publication
(10) Pub. No.: US 2024/0000795 A1
(43) Pub. Date: Jan. 4, 2024

(54) METHODS AND COMPOSITIONS RELATING TO CONTROLLING PSYCHEDELIC EFFECTS WITH SEROTONIN RECEPTOR MODULATORS

(71) Applicant: Terran Biosciences Inc., New York, NY (US)

(72) Inventor: Sam CLARK, New York, NY (US)

(21) Appl. No.: 17/945,865

(22) Filed: Sep. 15, 2022

Related U.S. Application Data
(60) Provisional application No. 63/357,470, filed on Jun. 30, 2022.

(51) Int. Cl. A61K 31/5513; A61K 31/519; A61K 31/554; A61K 45/06; A61P 25/14

(52) U.S. Cl. CPC A61K 31/5513; A61K 31/519; A61K 31/554; A61K 45/06; A61P 25/14

(57) ABSTRACT

Provided herein are compositions relating to psychedelics and serotonin receptor modulators. Further provided herein are methods of suppressing or halting hallucinogenic effects of a psychedelic and methods of treating a disease or disorder (e.g., depression or diseases or disorders related to depression) comprising administering psychedelics and serotonin receptor modulators.

### CROSS REFERENCE TO RELATED APPLICATIONS

This application claims the benefit of U.S. Provisional Patent Application No. 63/357,470, filed Jun. 30, 2022, the content of which is incorporated by reference herein in its entirety.

### BACKGROUND

Psychedelic substances have been used by humans for millennia for spiritual and medicinal purposes. Clinical research has recently begun to provide evidence supporting their use as therapeutics for numerous neuropsychiatric disorders, including obsessive-compulsive disorder, posttraumatic stress disorder, and treatment-resistant depression (TRD). However, the use of psychedelic substances such as psilocybin are not widely utilized for therapeutics because of the intense psychedelic-induced alterations in sensory perception and consciousness that they produce.

Psychedelics have been shown to have therapeutic benefits; however, the duration of activity often greatly exceeds the time for psychedelic-assisted psychotherapy. For example, while a typical therapy session might last up to several hours, LSD can have effects lasting 12 hours, and psilocybin can last 6 hours. Such long lasting effects of psychedelics may limit the number of patients that a doctor can treat per day and thus limits access to these important medicines.

Psychedelics induce hallucinations through the 2A receptor and it has been reported that pretreatment with selective 2A antagonist sufficient to pre-block the 2A receptor blocks the subjective effects of the psychedelic in animal models when the psychedelic is administered at timepoints after the selective 2A antagonist. However, whether selective 2A antagonists can end psychedelic effects (e.g., trips) when administered after the trip has already begun is unknown.

The therapeutic potential of psychedelics could be improved if the psychedelic response could be diminished without impairing the therapeutic response. Provided herein are compositions and methods relating to psychedelics and serotonin receptor modulators that provide therapeutic effects while reducing the psychedelic-induced alterations in sensory perception and consciousness.

### BRIEF SUMMARY

In one aspect, provided herein are methods of treating a disease or disorder in a subject in need thereof, the method comprising:
(a) administering to the subject a psychedelic, and
(b) administering to the subject olanzapine, risperidone, or quetiapine;
wherein olanzapine, risperidone, or quetiapine is administered after the administration of the psychedelic.

In certain embodiments, olanzapine, risperidone, or quetiapine is administered at most about 3 hours after the administration of the psychedelic.

In certain embodiments, olanzapine, risperidone, or quetiapine is administered at most about 2 hours after the administration of the psychedelic.

In certain embodiments, olanzapine, risperidone, or quetiapine is administered at most about 1 hour after the administration of the psychedelic.

In certain embodiments, olanzapine, risperidone, or quetiapine is administered at most about 0.5 hours after the administration of the psychedelic.

In certain embodiments, the disease or disorder is depression or a disease or disorder related to depression.

In certain embodiments, the depression is major depressive disorder, persistent depressive disorder, bipolar disorder, treatment resistant depression (TRD), postpartum depression, premenstrual dysphoric disorder, or seasonal affective disorder.

In certain embodiments, the disease or disorder is an anxiety disorder.

In certain embodiments, the anxiety disorder is generalized anxiety disorder, post-traumatic stress disorder, obsessive-compulsive disorder, panic disorder, social anxiety disorder, phobias, or borderline personality disorder.

In certain embodiments, the psychedelic is an agonist of serotonin receptor 2A.

In certain embodiments, the psychedelic is selected from psilocin, psilocybin, 4-Acetoxy-DMT, LSD, ALD-52, 1P-LSD, DMT, 5-MeO-DMT, 2C-B, ibogaine, MDMA, DOM, mescaline, (R)-MDMA, (S)-MDMA, MBDB, Methylone, (R)-methylone, (S)-methylone, MDEA, (S)-MDEA, (R)-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI.

In certain embodiments, the psychedelic is psilocybin.

In certain embodiments, the psychedelic is LSD.

In certain embodiments, the psychedelic is DMT or 5-MeO-DMT.

In certain embodiments, the psychedelic is O-acetyl psilocin.

In certain embodiments, the psychedelic is psilocin.

In certain embodiments, the psychedelic is a phenethylamine or a tryptamine.

In certain embodiments, olanzapine is administered.

In certain embodiments, risperidone is administered.

In certain embodiments, quetiapine is administered.

In certain embodiments, the psychedelic is administered orally, intravenously, subcutaneously, by inhalation, or by an injection.

In certain embodiments, olanzapine, risperidone, or quetiapine is administered orally, intravenously, subcutaneously, by inhalation, or by an injection.

In another aspect, provided herein are methods of reducing adverse effects of a psychedelic in the treatment of a disease or disorder in a subject in need thereof, the method comprising:
(a) administering to the subject a psychedelic, and
(b) administering to the subject olanzapine, risperidone, or quetiapine to posttreat the subject,
wherein olanzapine, risperidone, or quetiapine is administered after the administration of the psychedelic.

In certain embodiments, the olanzapine, risperidone, or quetiapine is administered at most about 3 hours after the administration of the psychedelic.

In certain embodiments, the psychedelic is selected from the group consisting of psilocin, psilocybin, 4-Acetoxy-DMT, LSD, ALD-52, 1P-LSD, DMT, 5-MeO-DMT, 2C-B, ibogaine, MDMA, DOM, mescaline, (R)-MDMA, (S)-MDMA, MBDB, Methylone, (R)-methylone, (S)-methylone, MDEA, (S)-MDEA, (R)-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI.

In certain embodiments, the disease or disorder is depression or a disease or disorder related to depression, or the disease or disorder is an anxiety disorder.

In yet another aspect, also provided herein are compositions comprising:
a) a psychedelic;
b) olanzapine, risperidone, or quetiapine; and
c) an excipient,
wherein olanzapine, risperidone, or quetiapine is released after the release of the psychedelic.

In certain embodiments, olanzapine, risperidone, or quetiapine is released at most about 3 hours after the release of the psychedelic.

In certain embodiments, the psychedelic is selected from the group consisting of psilocin, psilocybin, 4-Acetoxy-DMT, LSD, ALD-52, 1P-LSD, DMT, 5-MeO-DMT, 2C-B, ibogaine, MDMA, DOM, mescaline, (R)-MDMA, (S)-MDMA, MBDB, Methylone, (R)-methylone, (S)-methylone, MDEA, (S)-MDEA, (R)-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI.

In yet another aspect, provided herein are methods of treating a disease or disorder in a subject in need thereof, the method comprising administering to the subject the composition provided herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows dose-dependent effects of a single i.p. injection of LSD and psilocybin on head-twitch responses in mice. Mice were dosed intraperitoneally with vehicle (saline), psilocybin (1 mg/kg) or LSD (0.1 or 0.32 mg/kg). Following dosing, mice were replaced into their test cages and head-twitch responses continuously scored for 40 min.

FIGS. 2A-2B illustrate comparison of LSD induced head twitches in mice with and without administration of volinanserin after T=5 minutes. Mice were dosed intraperitoneally with LSD (0.32 mg/kg) and head-twitch responses were continuously scored for 5 min. After 5 min, mice were intravenously dosed with either vehicle (saline) or volinanserin (1 mg/kg). Cumulative head-twitches were measured every 2 minutes until 40 minutes after agonist dosing.

FIGS. 3A-3B illustrate comparison of LSD induced head twitches in mice with and without administration of volinanserin after T=10 minutes. Mice were dosed intraperitoneally with LSD (0.32 mg/kg) and head-twitch responses were continuously scored for 10 minutes. After 10 minutes, mice were intravenously dosed with either vehicle (saline) or volinanserin (1 mg/kg). Cumulative head-twitch behavior was measured every 2 minutes until 40 minutes after agonist dosing.

FIGS. 4A-4B illustrate comparison of LSD induced head twitches in mice with and without administration of volinanserin after T=8 minutes. Mice were dosed intraperitoneally with LSD (0.32 mg/kg) and head-twitch responses were continuously scored for 8 minutes. After 8 minutes, mice were intravenously dosed with either vehicle (saline) or volinanserin (1 mg/kg). Cumulative head-twitch behavior was measured every 2 minutes until 40 minutes after agonist dosing.

FIGS. 5A-5B illustrate comparison of LSD induced head twitches in mice with and without administration of ketanserin after T=8 minutes. Mice were dosed intraperitoneally with psilocybin (2 mg/kg) and head-twitch responses were continuously scored for 8 minutes. After 8 minutes, mice were intravenously dosed with either vehicle (DMSO:Cremophor:HPCD) or ketanserin (2 mg/kg). Cumulative head-twitch behavior was measured every 2 minutes until 40 minutes after agonist dosing.

FIGS. 6A-6B illustrate comparison of LSD induced head twitches in mice with and without administration of eplivanserin and pimavanserin after T=8 minutes. Mice were dosed intraperitoneally with LSD (0.32 mg/kg) and head-twitch responses were continuously scored for 8 minutes. After 8 minutes, mice were intravenously dosed with either vehicle (DMSO:HPCD [10:90]), eplivanserin (1 mg/kg), or pimavanserin (1 mg/kg). Cumulative head-twitch behavior was measured every 2 minutes until 40 minutes after agonist dosing.

FIGS. 7A-7B illustrate effects of eplivanserin administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with eplivanserin (1 mg/kg). Cumulative head-twitch behavior was measured every 2 minutes until 40 minutes after agonist dosing. As shown here, administration of eplivanserin (in n=6 mice) partially suppressed the head-twitch response.

FIGS. 8A-8B illustrate average effects of eplivanserin administered after T=8 minutes on LSD induced head twitches in mice. Protocol 6 S-TBS-011 and Protocol 14 S-TBS-021: Mice were dosed intraperitoneally with LSD (0.32 mg/kg) and head-twitch responses were continuously scored for 8 minutes. After 8 minutes, mice were intravenously dosed with either vehicle (DMSO:HPCD [10:90]) or eplivanserin (1 mg/kg). Cumulative head-twitch behavior was measured every 2 minutes until 40 minutes after agonist dosing. The plots show the average head twitches in mice dosed with LSD and eplivanserin in S-TBS-011 (n=3) and S-TBS-021 (n=6), compared to the average head twitches in mice dosed with LSD and the vehicle (DMSO:HPCD [10:90]) in S-TBS-011 and across all other studies with the same vehicle.

FIGS. 9A-9B illustrate effects of risperidone and pruvanserin administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with either vehicle (DMSO:HPCD [10:90]), risperidone (1 mg/kg), or pruvanserin (1 mg/kg). Cumulative head-twitch behavior was measured every 2 minutes until 40 minutes after agonist dosing.

FIGS. 10A-10B illustrate effects of nelotanserin, ritanserin, and olanzapine administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with either vehicle (DMSO:HPCD [10:90]), nelotanserin (1 mg/kg), ritanserin (1 mg/kg), or olanzepine (1 mg/kg). Cumulative head-twitch behavior was measured every 2 minutes until 40 minutes after agonist dosing.

FIGS. 11A-11B illustrate effects of quetiapine administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with either vehicle (saline) or quetiapine (1 mg/kg). Cumulative head-twitch behavior was measured every 2 minutes until 40 minutes after agonist dosing.

FIGS. 12A-12B illustrate effects of AC-279 administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with either vehicle (saline) or AC-279 (1 mg/kg). Cumulative head-twitch behavior was measured every 2 minutes until 40 minutes after agonist dosing.

FIGS. 13A-13B illustrate effects of flibanserin administered after T=8 minutes on LSD induced head twitches in mice. Mice were dosed intraperitoneally with LSD (0.32 mg/kg) and head-twitch responses were continuously scored for 8 minutes. After 8 minutes, mice were intravenously dosed with either vehicle (DMSO:HPCD) or flibanserin (1 mg/kg or 4 mg/kg). Cumulative head-twitch behavior was measured every 2 minutes until 40 minutes after agonist dosing.

FIGS. 14A-14B illustrate comparison of psilocybin induced head twitches in mice with and without administration of volinanserin after T=7 minutes. Mice were dosed intraperitoneally with psilocybin (2 mg/kg) and head-twitch responses were continuously scored for 7 minutes. After 7 min, mice were intravenously dosed with either vehicle (saline) or volinanserin (1 mg/kg). Cumulative head-twitches were measured every 2 minutes until 40 minutes after agonist dosing.

FIG. 15A-15B illustrate effects of eplivanserin and pimavanserin administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle 1 (DMSO:HPCD [10:90]), volinanserin (1 mg/kg), or pimavanserin (1 mg/kg). Cumulative head-twitch behavior was measured every 2 minutes until 40 minutes after agonist dosing.

FIGS. 16A-16B illustrate effects of pruvanserin and risperidone administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle 1 (DMSO:HPCD [10:90]), pruvanserin (1 mg/kg), or risperidone (1 mg/kg). Cumulative head-twitch behavior was measured every 2 minutes until 40 minutes after agonist dosing.

FIGS. 17A-17B illustrate effects of olanzapine, ritanserin, and nelotanserin administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle 1 (DMSO:HPCD [10:90]), olanzepine (1 mg/kg), ritanserin (1 mg/kg), or nelotanserin (1 mg/kg). Cumulative head-twitch behavior was measured every 2 minutes until 40 minutes after agonist dosing.

FIGS. 18A-18B illustrate effects of quetiapine administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle 1 (saline) or quetiapine (1 mg/kg). Cumulative head-twitch behavior was measured every 2 minutes until 40 minutes after agonist dosing. As shown here, administration of quetiapine completely suppressed the head-twitch response. 18A shows a graph.

FIGS. 19A-19B illustrate comparison of psilocybin induced head twitches in mice with and without administration of ketanserin after T=7 minutes. Mice were dosed intraperitoneally with psilocybin (2 mg/kg) and head-twitch responses were continuously scored for 7 minutes. After 7 minutes, mice were intravenously dosed with either vehicle (DMSO:cremophor:HPCD) or ketanserin (2 mg/kg). Cumulative head-twitch behavior was measured every 2 minutes until 40 minutes after agonist dosing.

FIGS. 20A-20B illustrate effects of AC-279 administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle (DMSO:HPCD [10:90]) or AC-279 (1 mg/kg). Cumulative head-twitch behavior was measured every 2 minutes until 40 minutes after agonist dosing.

FIGS. 21A-21B: Effects of flibanserin administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with flibanserin (1 mg/kg or 4 mg/kg). Cumulative head-twitch behavior was measured every 2 minutes until 40 minutes after agonist dosing. The average cumulative head twitches in mice dosed with psilocybin and then the same vehicle (DMSO:HPCD [10:90]) from the other studies is plotted for reference.

FIGS. 22A-22B illustrate effects of high dose nelotanserin administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle (DMSO:Kolliphor:HPCD (20% in water) [10:10:80]) or nelotanserin (4 mg/kg). Cumulative head-twitch behavior was measured every 2 minutes until 40 minutes after agonist dosing.

FIGS. 23A-23B illustrate effects of nelotanserin administered after T=7 minutes on psilocybin induced head twitches in mice. Mice were dosed intraperitoneally with psilocybin (2 mg/kg) and head-twitch responses were continuously scored for 7 minutes. After 7 minutes, mice were intravenously dosed with either vehicle 1 (DMSO:HPCD [10:90]) or nelotanserin (1 mg/kg) in S-TBS-016, or with either vehicle 2 (DMSO:Kolliphor:HPCD (20% in water) [10:10:80]) or nelotanserin (4 mg/kg) in S-TBS-023. Cumulative head-twitch behavior was measured every 2 minutes until 40 minutes after agonist dosing.

### DETAILED DESCRIPTION

Provided herein are compositions relating to psychedelics and serotonin receptor modulators. Further provided herein are methods of suppressing or halting hallucinogenic effects of a psychedelic and methods of treating a disease or disorder (e.g., depression or diseases or disorders related to depression) comprising administering psychedelics and serotonin receptor modulators. In some embodiments, a 5HT2A antagonist is administered after administration of a psychedelics (e.g., LSD or psilocybin), e.g., administered at a timepoint where the trip effect induced by the psychedelic reaches or is about to reach its peak effects. LSD and psilocybin are representative psychedelics of two main classes of psychedelics, DMT class (which also includes DMT, 5-MeO-DMT, etc.) and the ergot psychedelics class. Both LSD and psilocybin have shown therapeutic benefit in humans, and having potency in the mg range (psilocybin) or in the microgram range (LSD).

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “modified release” coating encompasses coatings that delay release, sustain release, extend release, prevent release, minimize release and/or otherwise prolong the release of a drug relative to formulations lacking such coatings which release a drug relatively quickly (i.e., “immediate release” compositions). The term “modified release” encompasses “sustained release,” “extended release,” “delayed release,” and the like. The term “modified release” is used interchangeably with “controlled release” or “delayed release”. The term “modified-release” or “delayed release” dosage composition refers broadly to a dosage form showing one or more modified-release properties, as described herein.

### Compositions

Psychedelics have the potential to be used for therapeutics for treating various neuropsychiatric disorders including depression. However, use of psychedelics for treatment are associated with negative effects such as psychedelic-induced alterations in sensory perception and consciousness. These negative effects would be greatly reduced if the psychedelic response could be diminished without impairing the therapeutic response. Described herein, in certain embodiments, are compositions comprising a psychedelic and a serotonin receptor modulator that provide therapeutic effects while reducing the effects associated with psychedelics. In some embodiments, the psychedelic and the serotonin receptor modulator are provided in a single oral dosage with two different release profiles such that the serotonin receptor modulator is released a certain time after the psychedelic. In some embodiments the psychedelic is given alone as a single oral dosage and the serotonin receptor modulator is given later as a separate single oral dosage. Compositions and methods as described herein, in certain embodiments, provide an anti-depressive effect without alteration in sensory perception and consciousness or with a minimal alteration in sensory perception and consciousness.

Many potential benefits for psychedelics in treating neurological conditions have been contemplated. Numerous neurological conditions are resistant to current treatments in a significant segment of the population and present widespread socioeconomic burden and personal suffering. Several problems have persisted in attempts to implement psychedelics into potential treatments for common and oftentimes intractable conditions such as depression and treatment-resistant depression. One such problem is the triggering of non-ordinary states of consciousness including hallucinogenic experiences in subjects in a which a psychedelic is administered. Another such problem is that the psychedelic-induced alterations in neural function which lead to the non-ordinary states of consciousness were believed to be the bases for many of the potentially contemplated therapeutic benefits of such psychedelics. The solutions provided herein solve these problems by demonstrating novel compositions and methods in which therapeutic benefits for treating or alleviating symptoms of a neurological condition using a psychedelic have been discovered that can be persist or be amplified by specifically blocking a component of the psychedelic which elicits the non-ordinary states of consciousness. This alleviates the undesirable and burdensome side-effects for utilizing psychedelics as therapeutics.

Described herein, in some embodiments, are compositions comprising a psychedelic and a serotonin receptor modulator, wherein the serotonin receptor modulator is released at most about 3 hours post to the release of the psychedelic. In some embodiments, the serotonin receptor modulator is released at most about 2 hours post to the release of the psychedelic. In some embodiments, the serotonin receptor modulator is released at most about 1.5 hours post to the release of the psychedelic. In some embodiments, the serotonin receptor modulator is released at most about 1 hour post to the release of the psychedelic. In some embodiments, the serotonin receptor modulator is released at about 1 hour post to the release of the psychedelic. In some embodiments, the serotonin receptor modulator is released at most about 5 minutes, 10 minutes, 20 minutes, 30 minutes, 40 minutes, 50 minutes, 1 hour, 1.25 hours, 1.5 hours, 2 hours, or 3 hours post to the release of the psychedelic. In some embodiments, the serotonin receptor modulator is released at most about 3 hours, 4 hours, 5 hours, 6 hours, 7 hours, 8 hours, 9 hours, or more than 9 hours post to the release of the psychedelic. In some embodiments, the serotonin receptor modulator is released in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour post to the release of the psychedelic.

In some embodiments, the serotonin receptor modulator is released at least about 0.5 hours post to the release of the psychedelic. In some embodiments, the serotonin receptor modulator is released at least about 1 hour post to the release of the psychedelic.

In some embodiments, the serotonin receptor modulator is released at most about 6 hours post to the release of the psychedelic. In some embodiments, the serotonin receptor modulator is released at most about 5 hours post to the release of the psychedelic. In some embodiments, the serotonin receptor modulator is released at most about 4 hours post to the release of the psychedelic. In some embodiments, the serotonin receptor modulator is released at most about 3 hours post to the release of the psychedelic.

In a preferred embodiment, the serotonin receptor modulator is released at about 1 hour to about 3 hours post to the release of the psychedelic.

Described herein, in some embodiments, are compositions comprising a psychedelic and a serotonin receptor modulator, wherein the serotonin receptor modulator is used to post-treat after the release of the psychedelic. In some embodiments, the serotonin receptor modulator is used to post-treat at most about 2 hours post to the release of the psychedelic. In some embodiments, the serotonin receptor modulator is used to posttreat at most about 1 hour post to the release of the psychedelic. In some embodiments, the serotonin receptor modulator is used to posttreat at most about 1.5 hours post to the release of the psychedelic. In some embodiments, the serotonin receptor modulator is used to posttreat at about 1 hour post to the release of the psychedelic. In some embodiments, the serotonin receptor modulator is used to posttreat at most about 5 minutes, 10 minutes, 20 minutes, 30 minutes, 40 minutes, 50 minutes, 1 hour, 1.25 hours, 1.5 hours, 2 hours, or 3 hours post to the release of the psychedelic. In some embodiments, the serotonin receptor modulator is used to posttreat at most about 3 hours, 4 hours, 5 hours, 6 hours, 7 hours, 8 hours, 9 hours, or more than 9 hours post to the release of the psychedelic. In some embodiments, the serotonin receptor modulator is used to posttreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour.

Described herein, in some embodiments, are compositions comprising a psychedelic and a serotonin receptor modulator, wherein the psychedelic modulates a serotonin receptor. In some embodiments, the psychedelic is a serotonergic psychedelic (also known as serotonergic hallucinogen). Serotonergic psychedelics are a subclass of psychedelic drugs with a method of action strongly tied to the neurotransmitter serotonin. Serotonin (often referred to as 5-HT, short for its full chemical name 5-hydroxytryptamine) is a naturally occurring neurotransmitter which is tied to positive mood, certain involuntary muscle control, and countless other functions, many of which are not yet fully understood. In some embodiments, the psychedelic is a serotonin receptor agonist. In some embodiments, the psychedelic is a partial agonist. In some embodiments, the serotonin receptor is serotonin receptor 1, serotonin receptor 2, serotonin receptor 4, serotonin receptor 5, serotonin receptor 6, or serotonin receptor 7. In some embodiments, the serotonin receptor is serotonin receptor TA, serotonin receptor 1B, serotonin receptor 1D, serotonin receptor 1E, serotonin receptor 1F, serotonin receptor 2A, serotonin receptor 2B, serotonin receptor 2C, serotonin receptor 4, serotonin receptor 5A, serotonin receptor 5B, serotonin receptor 6, or serotonin receptor 7. In some embodiments, the serotonin receptor is serotonin receptor 1B, serotonin receptor 4, serotonin receptor 6, or serotonin receptor 7.

Psychedelics

In some embodiments, the psychedelic is selected from the group consisting of psilocin, baeocystin, norbaeocystin, lisurgide, ibogaine, mescaline (3,4,5-trimethoxy-phenethylamine), phenethylamine (PEA), carboxamindotryptamine, proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5-dimethoxyphenethylamine), 3,4-Methylenedioxy-A (MDA), 3,4-methylenedioxy-N-ethylamphetamine (MDE), asymbescaline (3,4-Diethoxy-5-methoxyphenethylamine), mescaline-NBOMe, 1B-LSD, ETH-LAD, 1P-ETH-LAD, AL-LAD, LSZ, LSM-775, 1-(4-Bromofuro[2,3-f] [1]benzofuran-8-yl)propan-2-amine, 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromoamphetamine, 2,5-dimethoxy-4-bromo-o-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 2-Bromo-4,5-methylenedioxy-A (2-Br-4,5-MDA), 4-Bromo-3,5-dimethoxy-A (4-Br-3,5-DMA), 3,4-Dimethyl-2,5-dimethoxy-PEA (2C-G), 3,4-Trimethylene-2,5-dimethoxy-PEA (2C-G-3), 3,4-Trimethylene-2,5-dimethoxy-A (G-3), 3,4-Tetramethylene-2,5-dimethoxy-PEA (2C-G-4), 3,4-Tetramethylene-2,5-dimethoxy-A (G-4), 3,4-Norbornyl-2,5-dimethoxy-PEA (2C-G-5), 3,4-Norbornyl-2,5-dimethoxy-A (G-5), 1,4-Dimethoxynaphthyl-2-ethylamine (2C-G-N), 1,4-Dimethoxynaphthyl-2-isopropylamine (G-N), 2,5-Dimethoxy-PEA (2C-H), 4-Ethoxy-3,5-dimethoxy-A (3C-E), 4-Ethoxy-3,5-dimethoxy-PEA, 4-Benzyloxy-3,5-dimethoxy-A (3C-BZ), 4-Isopropoxy-2,5-dimethoxy-PEA (2C-O-4), 4-Methylseleno-2,5-dimethoxy-PEA (2C-SE), 4-Methylthio-2,5-dimethoxy-PEA (2C-T), 4-Isopropylthio-2,6-dimethoxy-PEA (psi-2C-T-4), 4-(2-Methoxyethylthio)-2,5-dimethoxy-PEA (2C-T-13), 4-Cyclopropylthio-2,5-dimethoxy-PEA (2C-T-15), 4-(s)-Butylthio-2,5-dimethoxy-PEA (2C-T-17), 4-Acetoxy-N-methyl-N-ethyltryptamine (4-AcO-MET), 4-Acetoxy-N-methyl-N-allyltryptamine (4-AcO-MALT), 4-Acetyloxy-N,N-diallyltryptamine (4-AcO-DALT), N,N,N-trimethyl-4-phosphoryloxytryptamine (aeruginascin), 4-Hydroxy-N,N,N-trimethyltryptamine, [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine, 6-Allyl-N,N-diethyl-NL, N,N-Dibutyl-T, N,N-Diethyl-T, N,N-Diisopropyl-T, alpha-methyl-T, 5-Methyoxy-alpha-methyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy 1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N,N-Dipropyl-4-hydroxy-T, N-Ethyl-4-hydroxy-N-methyl-T, 4-Hydroxy-N-isopropyl-N-methyl-T,4-Hydroxy-N-methyl-N-propyl-T, 4-Hydroxy-N,N-tetramethylene-T, Ibogaine, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, 2,N-Dimethyl-4,5-methylenedioxy-A, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, N-Isopropyl-4-methoxy-N-methyl-T, N-Isopropyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha Methyl-T, N-Ethyl-T, N-Methyl-T, 6-Propyl-N L, N,N-Tetramethylene-T, Tryptamine, 7-Methoxy-1-methyl-1, 2,3,4-tetrahydro-C, alpha,N-Dimethyl-5-methoxy-T, alpha-Ethyl-3,4,5-trimethoxy-PEA (AEM), 4-Methylthio-2,5-dimethoxy-A (ALEPH), 4-Ethylthio-2,5-dimethoxy-A (ALEPH-2), 4-Isopropylthio-2,5-dimethoxy-A (ALEPH-4), 4-Phenylthio-2,5-dimethoxy-A (ALEPH-6), 4-Propylthio-2,5-dimethoxy-A (ALEPH-7), 2,5-Dimethoxy-alpha-ethyl-4-methyl-PEA (ARIADNE), 4-Butoxy-3,5-dimethoxy-PEA, 2,5-Dimethoxy-4,N-dimethyl-A (BEATRICE), 2,5-Bismethylthio-4-methyl-A (BIS-TOM), 4-Bromo-2,5,beta-trimethoxy-PEA (BOB), 2,5,beta-Trimethoxy-4-methyl-PEA (BOD), beta-Methoxy-3,4-methylenedioxy-PEA (BOH), 2,5-Dimethoxy-beta-hydroxy-4-methyl-PEA (BOHD), 3,4,5,beta-Tetramethoxy-PEA (BOM), 4-Cy clopropylmethoxy-3,5-dimethoxy-PEA (CPM), 4-Trideuteromethyl-3,5-dimethoxy-PEA (4-D), 3,4,5-trimethoxy-beta,beta-dideuterophenethylamine (beta-D), 4-Methyl-3,5-Dimethoxy-PEA, 2,4-Dimethoxy-A (2,4-DMA), 2,5-Dimethoxy-A (2,5-DMA), 3,4-Dimethoxy-A (3,4-DMA), 2-(2,5-Dimethoxy-4-methylphenyl)-cyclopropylamine (DMCPA), 3,4-Dimethoxy-beta-hydroxy-PEA (DME), 2,5-Dimethoxy-3,4-methylenedioxy-A (DMMDA), 2,3-Dimethoxy-4,5-methylenedioxy-A (DMMDA-2), 3,4-Dimethoxy-PEA (DMPEA), 2,5-dimethoxy-4-(n)-amylamphetamine (DOAM), 4-(2-Fluoroethyl)-2,5-dimethoxy-A (DOEF), 4-Ethyl-2,5-dimethoxy-A (DOET), 4-Methyl-2,6-dimethoxy-A (psi-DOM), 4-Propyl-2,5-dimethoxy-A (DOPR), 2,4,5-Triethoxy-A (EEE),4-Diethoxy-5-methoxy-A (EEM), 2,5-Diethoxy-4-methoxy-A (EME), 2-Ethoxy-4,5-dimethoxy-A (EMM), N,alpha-diethyl-3,4-methylenedioxy-PEA (ETHYL-J), N-Ethyl-alpha-propyl-3,4-methylenedioxy-PEA (ETHYL-K), Benzofuran-2-methyl-5-methoxy-6-(2-aminopropane), Benzofuran-2,2-dimethyl-5-methoxy-6-(2-aminopropane), N-Hydroxy-N-methyl-3,4-methylenedioxy-A (FLEA), 3,4-Dimethyl-2,5-dimethoxy-A, 2,5-Dimethoxy-N-hydroxy-4-ethylthio-PEA (HOT-2), 2,5-Dimethoxy-N-hydroxy-4-(n)-propylthio-PEA (HOT-7), 2,5-Dimethoxy-N-hydroxy-4-(s)-butylthio-PEA (HOT-17), 2,5-Dimethoxy-N,N-dimethyl-4-iodo-A (IDNNA), 2,3,4-Trimethoxy-PEA (IM), 3,5-Dimethoxy-4-isopropoxy-PEA (IP), 5-Ethoxy-2-methoxy-4-methyl-A (IRIS), alpha-Ethyl-3,4-methylenedioxy-PEA, 3-Methoxy-4,5-methylenedioxy-PEA, 3-Methoxy-4,5-methylenedioxy-A (MMDA), 2-Methoxy-4,5-methylenedioxy-A (MMDA-2), 2-Methoxy-3,4-methylenedioxy-A (MMDA-3a), 4-Methoxy-2,3-methylenedioxy-A (MMDA-3b), 4-methoxyamphetamine, N-Allyl-3,4-methylenedioxy-A (MDAL), N-Butyl-3,4-methylenedioxy-A (MDBU), N-Benzyl-3,4-methylenedioxy-A (MDBZ), N-Cyclopropylmethyl-3,4-methylenedioxy-A (MDCPM), N,N-Dimethyl-3,4-methylenedioxy-A (MDDM), N-(2-Hy droxyethyl)-3,4-methylenedioxy-A, N-Isopropyl-3,4-methylenedioxy-A (MDIP), N-Methyl-3,4-ethylenedioxy-A (MDMC), N-Methoxy-3,4-methylenedioxy-A, N-(2-Methoxyethyl)-3,4-methylenedioxy-A, alpha,alpha,N-Trimethyl-3,4-methylenedioxy-PEA (MDMP), N-Hydroxy-3,4-methylenedioxy-A (MDOH), 3,4-Methylenedioxy-PEA, alpha,alpha-Dimethyl-3,4-methylenedioxy-PEA (MDPH), N-Propargyl-3,4-methylenedioxy-A (MDPL), N-Propyl-3,4-methylenedioxy-A (MDPR), 3,4-Dimethoxy-5-ethoxy-PEA (ME), 3-methoxy-4,5-Ethylenedioxy-A (MEDA), 2-Methoxy-4,5-diethoxy-A (MEE), 2,5-Dimethoxy-4-ethoxy-A (MEM), 3-Methoxy-4-ethoxy-PEA, 5-Bromo-2,4-dimethoxy-A, 5-Methylthio-2,4-dimethoxy-A, N-Methyl-2,5-dimethoxy-A, 4-Bromo-2,5-dimethoxy-N-methyl-A, N-Methyl-alpha-ethyl-3,4-methylenedioxy-PEA, N-Methyl-alpha-propyl-3,4-methylenedioxy-PEA, N-Methyl-4-methoxy-A, N-Methyl-2-methoxy-4,5-methylenedioxy-A, 2,4-Dimethoxy-5-ethoxy-A (MME), 3,4-Dimethoxy-5-propoxy-PEA (MP), 2,5-Dimethoxy-4-propoxy-A (MPM), 2-Methylthio-4,5-dimethoxy-A, 3,5-Dimethoxy-4-phenethyloxy-PEA (PE), 4-Propynyloxy-3,5-dimethoxy-PEA, 3,5-Diethoxy-4-methoxy-PEA, 3,4,5-Tetramethoxy-A, 4-Ethoxy-3-ethylthio-5-methoxy-PEA, 3-Ethoxy-4-ethylthio-5-methoxy-PEA, 3,4-Diethoxy-5-methylthio-PEA, 4-Thiobutoxy-3,5-dimethoxy-PEA, 4-Ethoxy-5-methoxy-3-methylthio-PEA (3-TE), 3,5-Dimethoxy-4-ethylthio-PEA (4-TE), 2-Methylthio-3,4-dimethoxy-PEA (2-TIM), 3-Methylthio-2,4-dimethoxy-PEA (3-TIM), 4-Methylthio-2,3-dimethoxy-PEA (4-TIM), 3-Methylthio-4,5-dimethoxy-PEA (3-TM), 4-Methylthio-3,5-dimethoxy-PEA (4-TM), 3,4,5-Trimethoxy-A (TMA), 2,4,5-Trimethoxy-A (TMA-2), 2,3,4-Trimethoxy-A (TMA-3), 2,3,5-Trimethoxy-A (TMA-4), 2,3,6-Trimethoxy-A (TMA-5), 2,4,6-Trimethoxy-A (TMA-6), 4,5-Dimethoxy-3-ethylthio-PEA (3-TME), 3-Ethoxy-5-methoxy-4-methylthio-PEA (4-TME), 3-Ethoxy-4-methoxy-5-methylthio-PEA (5-TME), 2-Methylthio-3,4-methylenedioxy-A, 4,5-Thiomethyleneoxy-2-methoxy-A, 2,4,5-Trimethoxy-PEA, 4-Ethyl-5-methoxy-2-methylthio-A (2-TOET), 4-Ethyl-2-methoxy-5-methylthio-A (5-TOET), 4-Ethyl-2-methoxy-5-methylthio-A (2-TOM), 2-Methoxy-4-methyl-5-methylthio-A (5-TOM), 2-Methoxy-4-methyl-5-methylsulfinyl-A (TOMSO), 4-Propylthio-3,5-dimethoxy-PEA (TP), 3,4,5-Triethoxy-PEA (TRIS), 3-Ethoxy-5-ethylthio-4-methoxy-PEA (3-TSB), 3,5-Diethoxy-4-methylthio-PEA (4-TSB), 4,5-Diethoxy-3-ethylthio-PEA (3-T-TRIS), 3,5-Diethoxy-4-ethylthio-PEA (4-T-TRIS), 2-(2-Chlorophenyl)-2-(methylamino)cyclohexanone (ketamine), 5-methoxy-2,3-dihydro-1H-inden-2-amine (MEAI), N-methyl-N-allyltryptamine (MALT),N-ethyl-N-propyltryptamine (EPT), 5-Methoxy-N,N-diallyltryptamine (5-MeO-DALT), 6-Methoxy-N,N-dimethyltryptamine (6-MeO-DMT), 6-fluoro-N,N,-dimethyltryptamine (6-Fluoro-DMT), N-methyl-N-propyltryptamine (MPT), N-Methyl-N-isopropyltryptamine (MiPT), N,N-Dimethyl-N-allyltryptamine (DMALT), 4-Acetoxy-N,N,N-trimethyltryptamine (4-AcO-TMT), 4-Acetoxy-N,N-dimethyl-N-ethyltryptamine (4-OAc-DMET), 4-Acetoxy-N,N-dimethyl-N-propyltryptamine (4-AcO-DMPT), N-(4-bromophenyl)adamantan-2-amine (bromantane), 3-(6-(4-fluoro-3-methoxyphenoxy)pyrimidin-4-yl)-5,5-dimethylimidazolidine-2,4-dione, N-(4-((2-fluorobenzyl)oxy)benzyl)-2-(trifluoromethyl)thiazole-4-carboxamide, 4-{trans-2-[4-(3-Fluorophenyl)pyrimidin-2-yl]cyclopropyl}benzenesulfonamide, sodium 6-methoxy-2-methyl-3-(3,4,5-trimethoxybenzoyl)benzofuran-7-yl phosphate, 1-ethyl-6-(indan-2-ylamino)-3-(morpholine-4-carbonyl)-1,8-naphthyridin-4-one, 4-((1S,3S)-3-(5-cyclopentyl-1,2,4-oxadiazol-3-yl)-2,2-dimethylcyclopropyl)benzenesulfonamide, (2S,5R)-5-(4-((1-(5-fluoro-2-(trifluoromethoxy)phenyl)-1H-tetrazol-5-yl)oxy)phenyl)pyrrolidine-2-carboxamide, N-(4-fluorophenethyl)-3-methylisoxazole-4-sulfonamide, (7-hydroxy-6-methoxy-2-methylbenzofuran-3-yl)(3,4,5-trimethoxyphenyl)methanone(7-hydroxy-6-methoxy-2-methylbenzofuran-3-yl)(3,4,5-trimethoxyphenyl)methanone, 6-((2,3-dihydro-1H-inden-2-yl)amino)-1-ethyl-3-(1-methyl-1H-imidazol-2-yl)-1,8-naphthyridin-4(1H)-one, 2-(2-(Allyloxy)-5-fluorophenyl)cyclopropyl)methanamine, 1,5-dimethyl-N-(2-(trifluoromethyl)pyridin-4-yl)-1H-indole-3-carboxamide, (2-(5-Fluoro-2-(2-fluoroethoxy)phenyl)cyclopropyl)methanamine, (2-(5-Chloro-2-(2-fluoroethoxy)phenyl)cyclopropyl)methanamine, (2-(5-Chloro-2-((2-fluoroallyl)oxy)phenyl)cyclopropyl)methanamine, 5,6-dimethoxy-2,3-dihydro-1H-inden-2-amine, N-N-disisopropyltryptamine-4-glutarate, 2-methoxy-7-methyl-5,6,7,8,9,10-hexahydropyrido[3′,2′:4,5]pyrrolo[2,3-d]azepine, 1-(5-methoxy-1H-indol-1-yl)-N,N,2-trimethylpropan-2-amine, 2-(5-methoxy-1H-pyrrolo[2,3-c]pyridin-1-yl)-N,N-dimethylethanamine, (R)-N,N-diethyl-1,3,4,5-tetrahydrobenzo[cd]indol-4-amine, (S)-N,N-diethyl-1,3,4,5-tetrahydrobenzo[cd]indol-4-amin, 2-(4-allyl-2,5-dimethoxyphenyl)ethanamine, N-Ethyl-2-(5-Fluoro-1H-Indol-3-YL)-N-Methylethan-1-Amine, (R)-2-(methylamino)-2-phenylcyclohexanone, (R)-2-(D3-methylamino)-2-phenylcyclohexanone, (S)-2-(methylamino)-2-phenylcyclohexanone, (S)-2-(D3-methylamino)-2-phenylcyclohexanone, (S)-3-(2,5-dimethoxy-4-(trifluoromethyl)phenyl)piperidine, 3-methyl-metheathinone (3-MMC), 3-(2-(Bis(Methyl-D3)Amino)Ethyl-1,1,2,2-D4)-1H-Indol-4-YL(9Z,12Z)-Octadeca-9,12-Dienoate, (R)-3-((1-(Methyl-d3)Pyrolidin-2-YL)Methyl)-1H-Indol-4-OL, 5-(2-methylaminopropyl)benzofuran, 6-(2-methylaminopropyl)benzofuran, 2-chloro-N,N,-dimethyltryptamine,2-bromo-N,N,-dimethyltryptamine, 2-bromo-4-acetoxy-N,N-dimethyltryptamine, 2-chloro-4-methoxy-N,N,-dimethyltryptamine, 1-(3-(2-(dimethylamino)ethyl)-1H-indol-4-yl)-N-methylmethanesulfonamide, DMT-alpha,alpha-d2 (DMT-d2), psilocin-alpha,alpha-d2 (psilocin-d2), aeruginascin-alpha-alpha-d2 (aeruginascin-d2), razoxane, dexrazoxane, N-Allyl-3,4-methylenedioxy-amphetamine (MDAL), N-Butyl-3,4-methylenedioxyamphetamine (MDBU), N-Benzyl-3,4-methylenedioxy amphetamine (MDBZ), N-Cyclopropylmethyl-3,4-methylenedioxyamphetamine (MDCPM), N,N-Dimethyl-3,4-methylenedioxyamphetamine (MDDM), N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC), N-Methoxy-3,4-methylenedioxyamphetamine (MDMEO), N-(2-Methoxyethyl)-3,4-methylenedioxyamphetamine (MDMEOET), alpha,alpha,N-Trimethyl-3,4-methylenedioxyphenethylamine (MDMP), 3,4-Methylenedioxy-N-methylphentermine, N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine), N-Propargyl-3,4-methylenedioxyamphetamine (MDPL), 3,4-methylenedioxy-N-methyl-a-ethylphenylethylamine,3,4-Methylenedioxyamphetamine (MDA), Ethylone, (also known as 3,4-methylenedioxy-N ethylcathinone), and N-Propyl-3,4 methylenedioxy amphetamine (MDPR), or a pharmaceutically acceptable salt, solvate, metabolite, deuterated analogue, derivative, prodrug, or combinations thereof.

In some embodiments, the psychedelic is selected from the group consisting of psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, 0-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), and asymbescaline (3,4-Diethoxy-5-methoxyphenethylamine). In some embodiments, the psychedelic is psilocybin or psilocin. In some embodiments, the psychedelic is [3-(2-dimethylaminoethyl)-1H-indol-4-yl]dihydrogen phosphate, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl]dihydrogen phosphate, 4-hydroxytryptamine, [3-(2-trimethylaminoethyl)-1H-indol-4-yl]dihydrogen phosphate, or 4-hydroxy-N,N,N-trimethyltryptamine. In some embodiments, the psychedelic is 1-acetyl LSD (ALD-52). In some embodiments, the psychedelic is O-acetyl psilocin (psilacetin).

In certain embodiments, the psychedelic is selected from psilocin, psilocybin, 4-Acetoxy-DMT, LSD, ALD-52, 1P-LSD, DMT, 5-MeO-DMT, 2C-B, ibogaine, MDMA, DOM, mescaline, (R)-MDMA, (S)-MDMA, MBDB, Methylone, (R)-methylone, (S)-methylone, MDEA, (S)-MDEA, (R)-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI.

In some embodiments, the psychedelic is LSD. In some embodiments, the LSD derivative is 1P-LSD, 1B-LSD, ETH-LAD, 1P-ETH-LAD, AL-LAD, LSZ, LSM-775, 1-(4-Bromofuro[2,3-f] [1]benzofuran-8-yl)propan-2-amine.

In some embodiments, the psychedelic is mescaline. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

In some embodiments, the psychedelic is a phenethylamine or a tryptamine, selected from 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-o-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl]dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl]dihydrogen phosphate, and 4-hydroxy-N,N,N-trimethyltryptamine, 6-Allyl-N,N-diethyl-NL, N,N-Dibu-tyl-T, N,N-Diethyl-T, N,N-Diisopropyl-T, 5-Methyoxy-alpha-methyl-T, N,N-Dimethyl-T, 2,alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methy 1-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1,2,3,4-tetrahydro-C, and alpha,N-Dimethyl-5-methoxy-T.

In some embodiments, the psychedelic is selected from the group consisting of

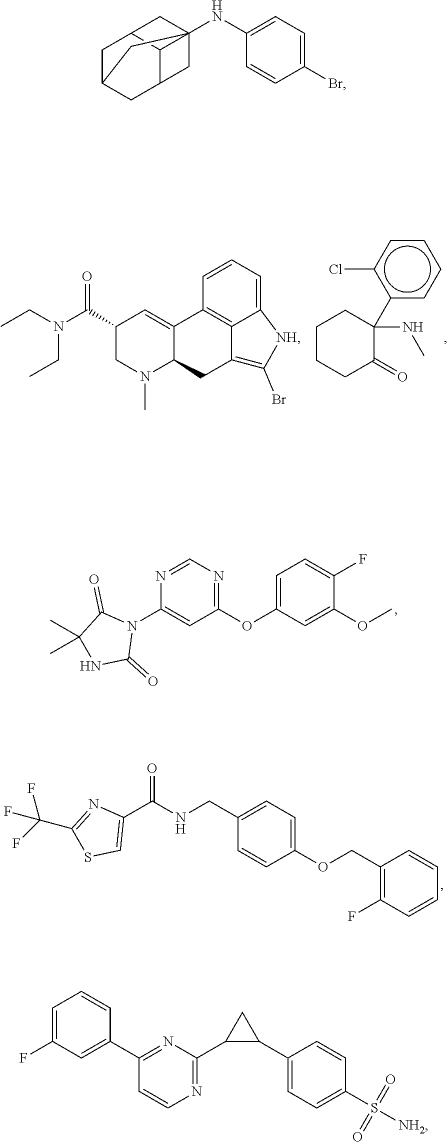

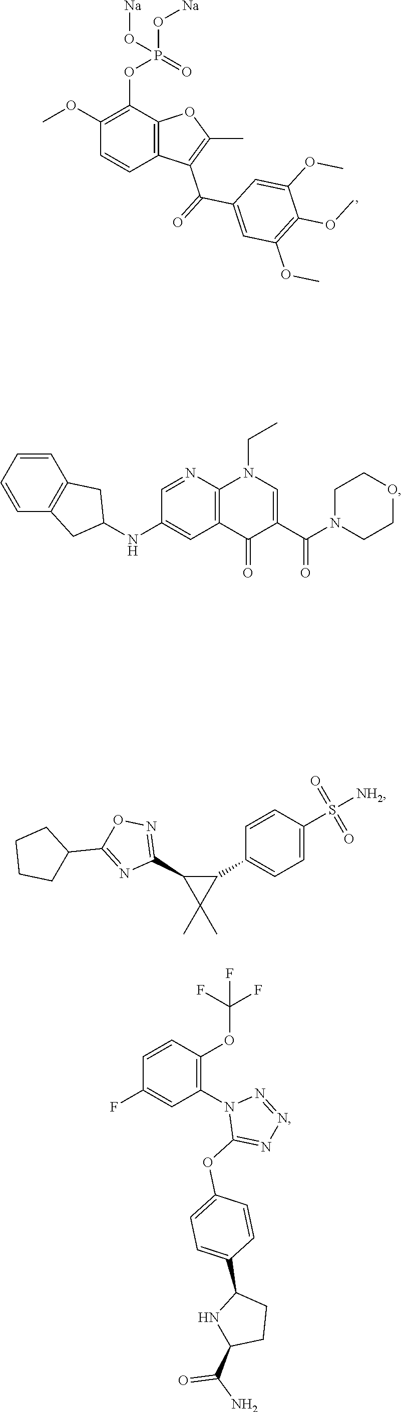

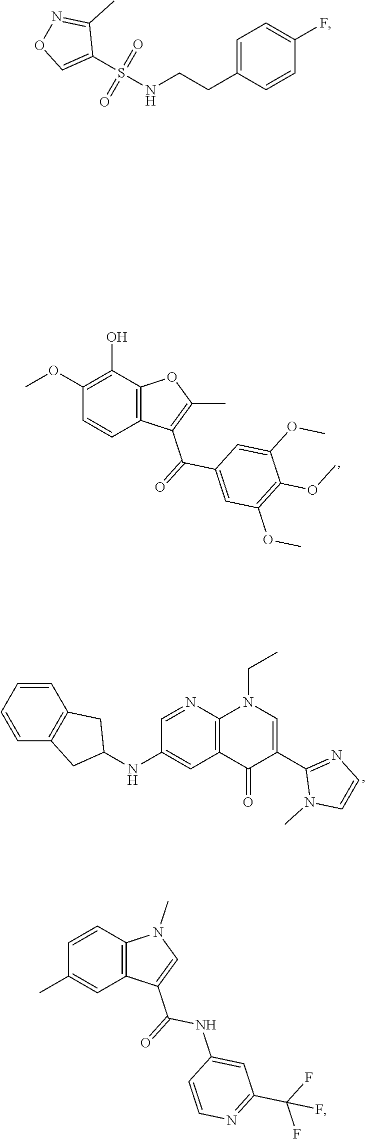

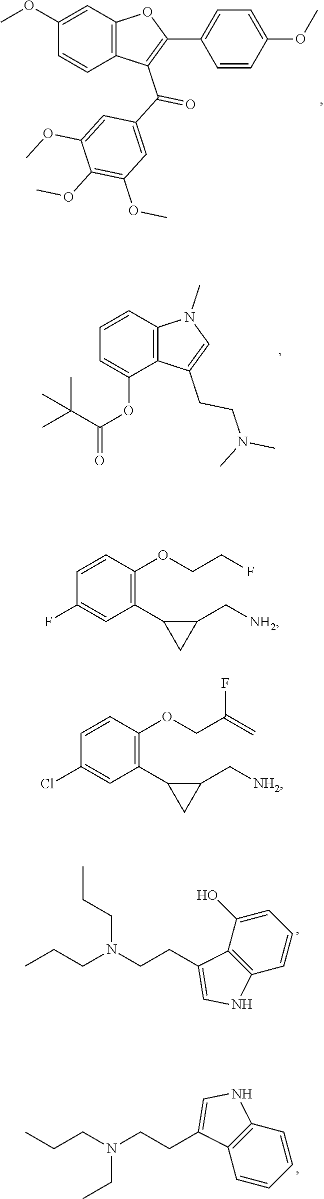

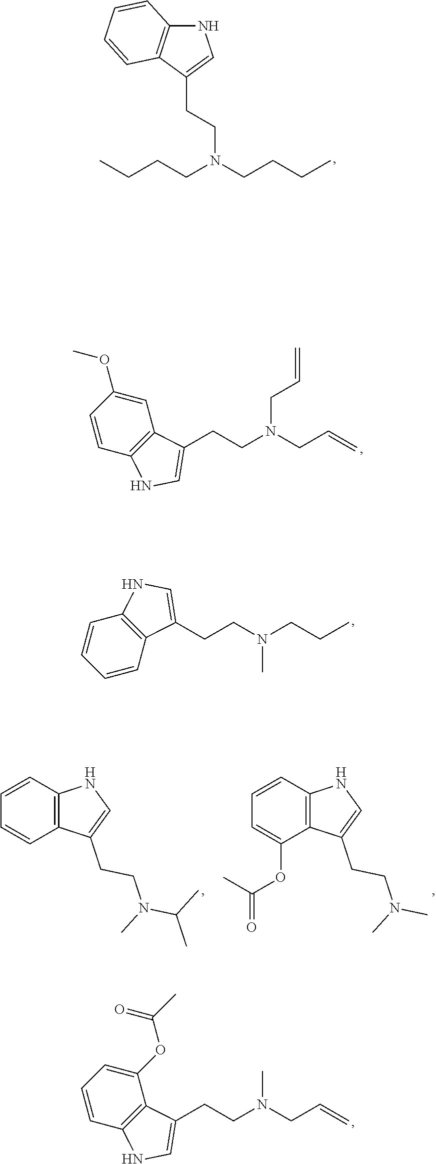

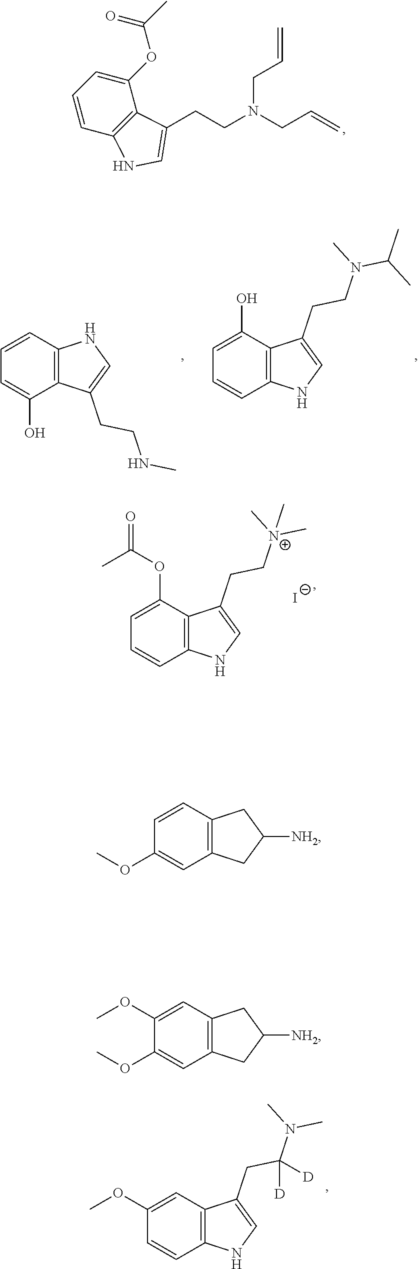

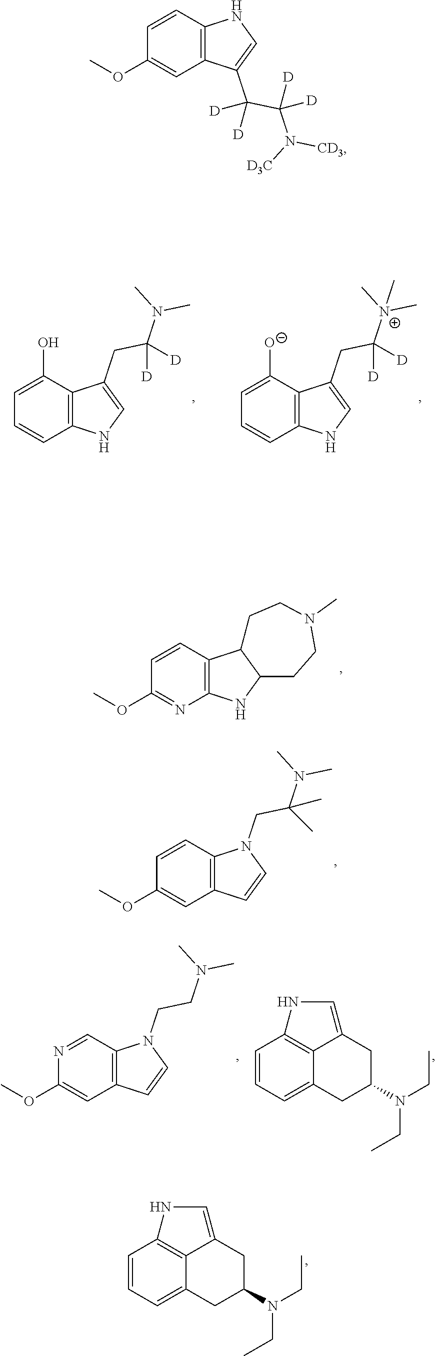

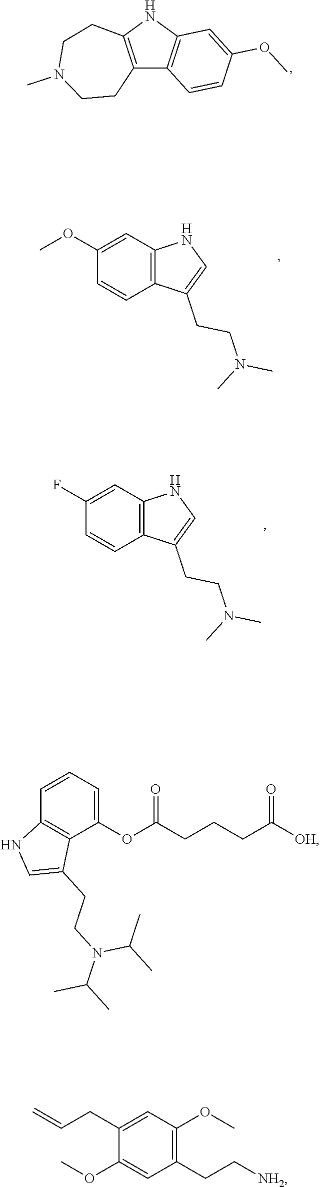

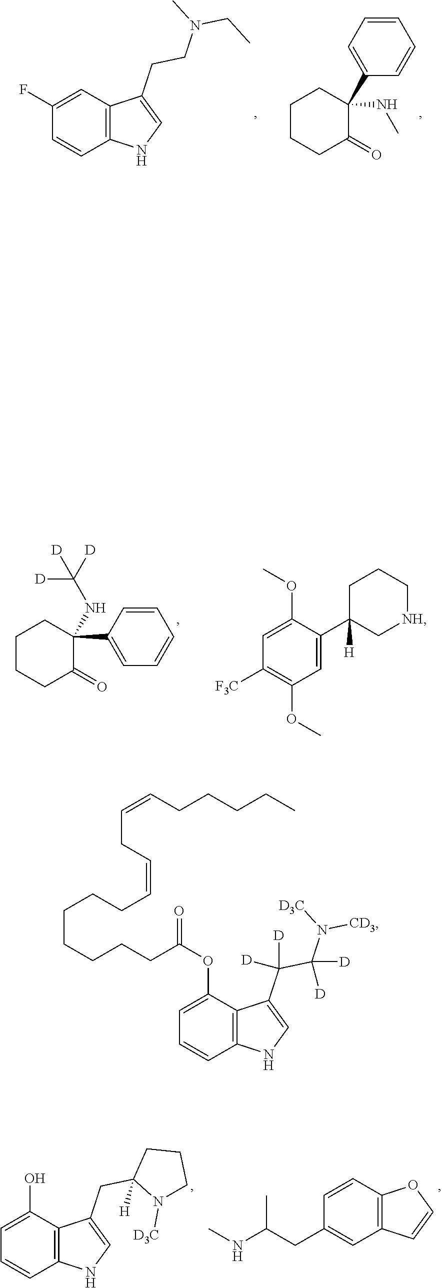

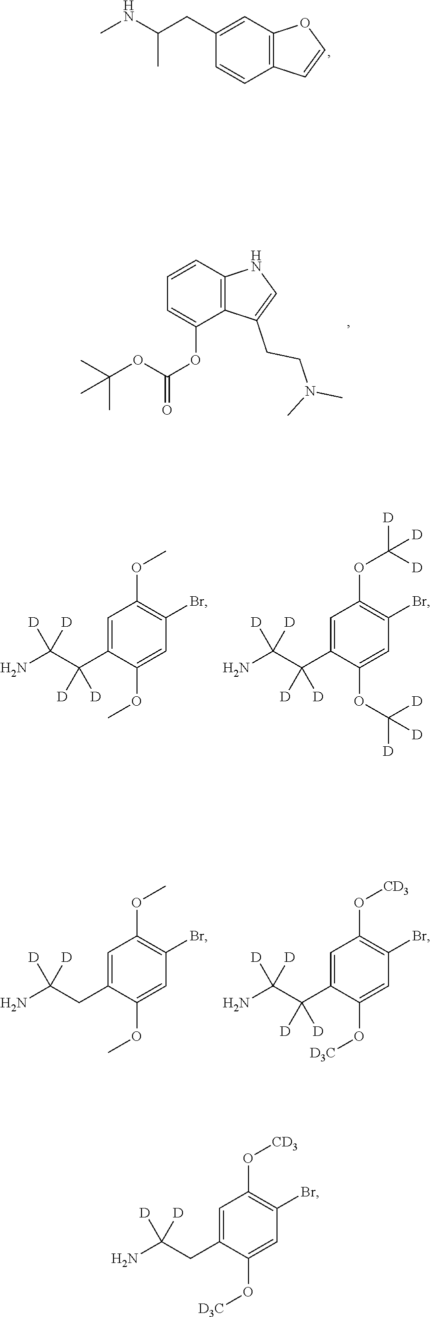

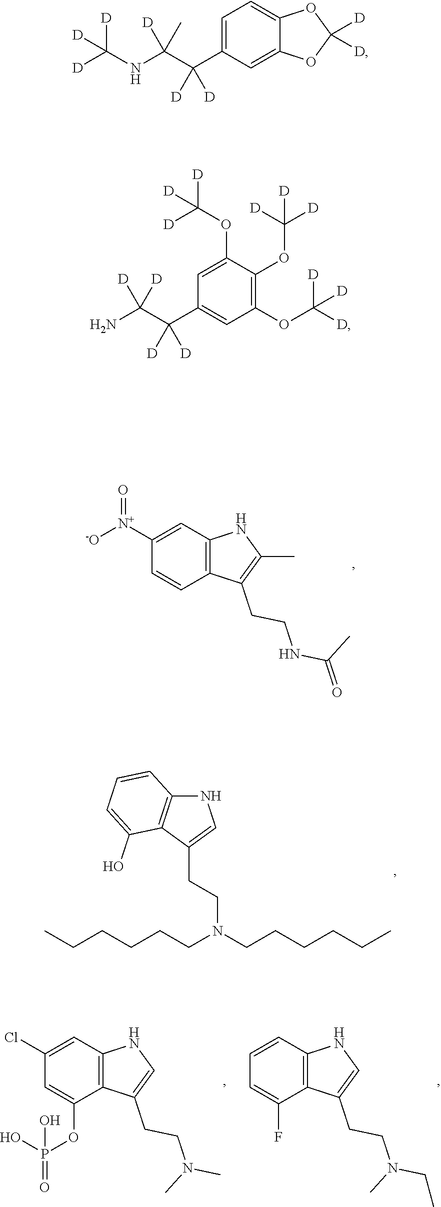

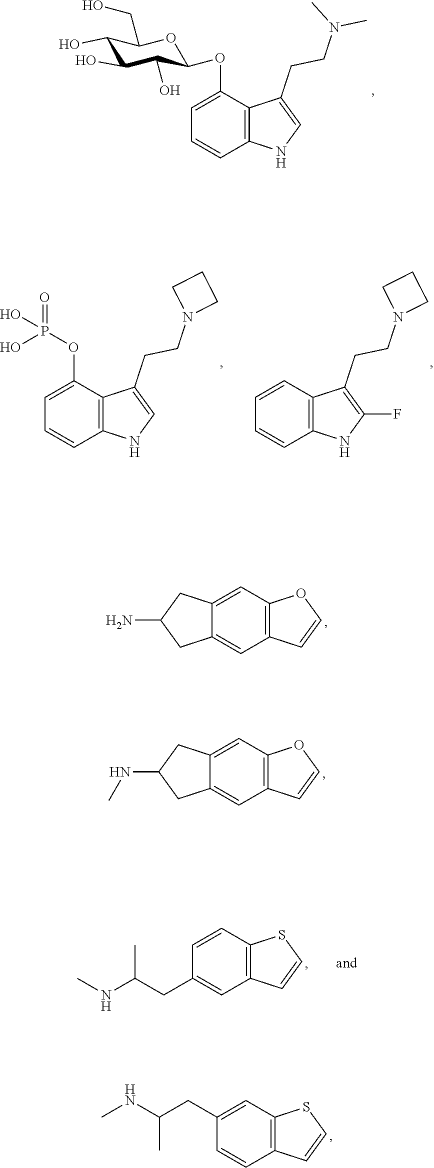

or a pharmaceutically acceptable salt, ester, solvate, metabolite, deuterated analogue, derivative, prodrug, or combinations thereof.

In some embodiments, the psilocybin or psilocin is present in the form of an extract from a mushroom and/or truffle (sclerotium). In some embodiments, the mushroom or truffle is from the genus Psilocybe, Gymnopilus, Panaeolus, Copelandia, Hypholoma, Pluteus, Inocybe, Conocybe, Panaeolina, Gerronema, Agrocybe, Galerina and/or Mycena. In some embodiments, the mushroom or truffle is P. azurescens, P. semilanceata, P. cyanescens, P. cubensis, P. subcubensis, P. tampanensis, P. mexicana, P. atlantis, and/or P. semilanceata.

In some embodiments, the psychedelic is selected from the group consisting of psilocybin, 4-Acetoxy-DMT, LSD, ALD-52, 1P-LSD, DMT, 5-MeO-DMT, 2C-B, MDMA, R-MDMA, S-MDMA, DOM, MBDB, Methylone, R-Methylone, S-methylone, 2-Br-LSD, tabernanthalog, AAZ-A-154, MDAI, MDEA, S-MDEA, and GHB or sodium oxybate.

Described herein, in some embodiments, are compositions comprising a psychedelic and a serotonin receptor modulator, wherein the serotonin receptor modulator is a serotonin receptor antagonist. Further described herein, in some embodiments, are compositions comprising a psychedelic and a serotonin receptor modulator, wherein the serotonin receptor modulator is a serotonin receptor inverse agonist. Further described herein, in some embodiments, are compositions comprising a psychedelic and a serotonin receptor modulator, wherein the serotonin receptor modulator is a serotonin receptor allosteric modulator. In some embodiments, the serotonin receptor is serotonin receptor 1, serotonin receptor 2, serotonin receptor 4, serotonin receptor 5, serotonin receptor 6, or serotonin receptor 7. In some embodiments, the serotonin receptor is serotonin receptor 1A, serotonin receptor 1B, serotonin receptor 1D, serotonin receptor 1E, serotonin receptor 1F, serotonin receptor 2A, serotonin receptor 2B, serotonin receptor 2C, serotonin receptor 4, serotonin receptor 5A, serotonin receptor 5B, serotonin receptor 6, or serotonin receptor 7. In some embodiments the serotonin receptor is serotonin receptor 2A.

### Serotonin Receptor Modulators

In some embodiments, the serotonin receptor modulator described herein is selected from ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, and lumateperone.

In some embodiments, the serotonin receptor modulator described herein comprises kMDL-11,939, eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, MDL-100,907, pimavanserin, nelotanserin, lorcaserin, flibanserin, and roluperiodone. In some embodiments, the serotonin receptor modulator is ketanserin. In some embodiments, the serotonin receptor modulator is pimavanserin.

In some embodiments, the serotonin receptor modulator is ketanserin.

In some embodiments, the serotonin receptor modulator is pimavanserin.

In some embodiments, the serotonin receptor modulator is eplivanserin. Co-Administration of psychedelic and serotonin receptor modulator

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI.

In some embodiments, the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is eplivanserin or volinanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is eplivanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is volinanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ritanserin, pimavanserin, nelotanserin, pruvanserin, and flibanserin.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ketanserin, pimavanserin, nelotanserin, pruvanserin, flibanserin, olanzapine, quetiapine, and risperidone.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, pimavanserin, nelotanserin, pruvanserin, flibanserin, olanzapine, quetiapine, and risperidone.

In some embodiments, the psychedelic is selected from the group consisting of psilocybin, 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of pimavanserin, nelotanserin, pruvanserin, flibanserin, olanzapine, quetiapine, and risperidone.

In some embodiments, the psychedelic is selected from the group consisting of 4-Acetoxy-DMT, LSD, ALD-52, 1P-LSD, DMT, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is selected from the group consisting of 4-Acetoxy-DMT, LSD, ALD-52, 1P-LSD, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is selected from the group consisting of 4-Acetoxy-DMT, ALD-52, 1P-LSD, 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is Psilocybin, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is LSD, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is DMT, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 5-MeO-DMT, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 4-Acetoxy-DMT, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is ALD-52, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 1P-LSD, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 2C-B, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is ibogaine, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is MDMA, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is DOM, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is mescaline, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is R-MDMA, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is S-MDMA, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is MBDB, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is methylone, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is R-methylone, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is S-methylone, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is MDEA, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is S-MDEA, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is N-Ethyl-2-(5-Fluoro-1H-Indol-3YL)-N-Methylethan-1-Amine, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 4-OH-DiPT hemi-glutarate, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 5,6-Dimethoxy-2-Aminoindane, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 5-Methoxy-2-Aminoindane, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is 2-Br-LSD, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is MDAI, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is psilocybin, and the serotonin receptor modulator is eplivanserin.

In some embodiments, the psychedelic is LSD, and the serotonin receptor modulator is eplivanserin.

In some embodiments, the psychedelic is DMT, and the serotonin receptor modulator is eplivanserin.

In some embodiments, the psychedelic is 5-MeO-DMT, and the serotonin receptor modulator is eplivanserin.

In some embodiments, the psychedelic is 4-Acetoxy-DMT, and the serotonin receptor modulator is eplivanserin.

In some embodiments, the psychedelic is ALD-52, and the serotonin receptor modulator is eplivanserin.

In some embodiments, the psychedelic is 1P-LSD, and the serotonin receptor modulator is eplivanserin.

In some embodiments, the psychedelic is 2C-B, and the serotonin receptor modulator is eplivanserin.

In some embodiments, the psychedelic is ibogaine, and the serotonin receptor modulator is eplivanserin.

In some embodiments, the psychedelic is MDMA, and the serotonin receptor modulator is eplivanserin.

In some embodiments, the psychedelic is DOM, and the serotonin receptor modulator is eplivanserin.

In some embodiments, the psychedelic is mescaline, and the serotonin receptor modulator is eplivanserin.

In some embodiments, the psychedelic is S-MDMA, and the serotonin receptor modulator is eplivanserin.

In some embodiments, the psychedelic is R-MDMA, and the serotonin receptor modulator is eplivanserin.

In some embodiments, the psychedelic is MBDB, and the serotonin receptor modulator is eplivanserin.

In some embodiments, the psychedelic is methylone, and the serotonin receptor modulator is eplivanserin.

In some embodiments, the psychedelic is R-methylone, and the serotonin receptor modulator is eplivanserin.

In some embodiments, the psychedelic is S-methylone, and the serotonin receptor modulator is eplivanserin.

In some embodiments, the psychedelic is MDEA, and the serotonin receptor modulator is eplivanserin.

In some embodiments, the psychedelic is S-MDEA, and the serotonin receptor modulator is eplivanserin.

In some embodiments, the psychedelic is N-Ethyl-2-(5-Fluoro-1H-Indol-3YL)-N-Methylethan-1-Amine, and the serotonin receptor modulator is eplivanserin.

In some embodiments, the psychedelic is 4-OH-DiPT hemi-glutarate, and the serotonin receptor modulator is eplivanserin.

In some embodiments, the psychedelic is 5,6-Dimethoxy-2-Aminoindane, and the serotonin receptor modulator is eplivanserin.

In some embodiments, the psychedelic is 5-Methoxy-2-Aminoindane, and the serotonin receptor modulator is eplivanserin.

In some embodiments, the psychedelic is 2-Br-LSD, and the serotonin receptor modulator is eplivanserin.

In some embodiments, the psychedelic is MDAI, and the serotonin receptor modulator is eplivanserin.

In some embodiments, the psychedelic is psilocybin, and the serotonin receptor modulator is volinanserin.

In some embodiments, the psychedelic is LSD, and the serotonin receptor modulator is volinanserin.

In some embodiments, the psychedelic is DMT, and the serotonin receptor modulator is volinanserin.

In some embodiments, the psychedelic is 5-MeO-DMT, and the serotonin receptor modulator is volinanserin.

In some embodiments, the psychedelic is 4-Acetoxy-DMT, and the serotonin receptor modulator is volinanserin.

In some embodiments, the psychedelic is ALD-52, and the serotonin receptor modulator is volinanserin.

In some embodiments, the psychedelic is 1P-LSD, and the serotonin receptor modulator is volinanserin.

In some embodiments, the psychedelic is 2C-B, and the serotonin receptor modulator is volinanserin.

In some embodiments, the psychedelic is ibogaine, and the serotonin receptor modulator is volinanserin.

In some embodiments, the psychedelic is MDMA, and the serotonin receptor modulator is volinanserin.

In some embodiments, the psychedelic is DOM, and the serotonin receptor modulator is volinanserin.

In some embodiments, the psychedelic is mescaline, and the serotonin receptor modulator is volinanserin.

In some embodiments, the psychedelic is S-MDMA, and the serotonin receptor modulator is volinanserin.

In some embodiments, the psychedelic is R-MDMA, and the serotonin receptor modulator is volinanserin.

In some embodiments, the psychedelic is MBDB, and the serotonin receptor modulator is volinanserin.

In some embodiments, the psychedelic is methylone, and the serotonin receptor modulator is volinanserin.

In some embodiments, the psychedelic is R-methylone, and the serotonin receptor modulator is volinanserin.

In some embodiments, the psychedelic is S-methylone, and the serotonin receptor modulator is volinanserin.

In some embodiments, the psychedelic is MDEA, and the serotonin receptor modulator is volinanserin.

In some embodiments, the psychedelic is S-MDEA, and the serotonin receptor modulator is volinanserin.

In some embodiments, the psychedelic is N-Ethyl-2-(5-Fluoro-TH-Indol-3YL)-N-Methylethan-1-Amine, and the serotonin receptor modulator is volinanserin.

In some embodiments, the psychedelic is 4-OH-DiPT hemi-glutarate, and the serotonin receptor modulator is volinanserin.

In some embodiments, the psychedelic is 5,6-Dimethoxy-2-Aminoindane, and the serotonin receptor modulator is volinanserin.

In some embodiments, the psychedelic is 5-Methoxy-2-Aminoindane, and the serotonin receptor modulator is volinanserin.

In some embodiments, the psychedelic is 2-Br-LSD, and the serotonin receptor modulator is volinanserin.

In some embodiments, the psychedelic is MDAI, and the serotonin receptor modulator is volinanserin.

In some embodiments, the psychedelic is psilocybin, and the serotonin receptor modulator is ketanserin.

In some embodiments, the psychedelic is LSD, and the serotonin receptor modulator is ketanserin.

In some embodiments, the psychedelic is DMT, and the serotonin receptor modulator is ketanserin.

In some embodiments, the psychedelic is 5-MeO-DMT, and the serotonin receptor modulator is ketanserin.

In some embodiments, the psychedelic is 4-Acetoxy-DMT, and the serotonin receptor modulator is ketanserin.

In some embodiments, the psychedelic is ALD-52, and the serotonin receptor modulator is ketanserin.

In some embodiments, the psychedelic is 1P-LSD, and the serotonin receptor modulator is ketanserin.

In some embodiments, the psychedelic is 2C-B, and the serotonin receptor modulator is ketanserin.

In some embodiments, the psychedelic is ibogaine, and the serotonin receptor modulator is ketanserin.

In some embodiments, the psychedelic is MDMA, and the serotonin receptor modulator is ketanserin.

In some embodiments, the psychedelic is DOM, and the serotonin receptor modulator is ketanserin.

In some embodiments, the psychedelic is mescaline, and the serotonin receptor modulator is ketanserin.

In some embodiments, the psychedelic is S-MDMA, and the serotonin receptor modulator is ketanserin.

In some embodiments, the psychedelic is R-MDMA, and the serotonin receptor modulator is ketanserin.

In some embodiments, the psychedelic is MBDB, and the serotonin receptor modulator is ketanserin.

In some embodiments, the psychedelic is methylone, and the serotonin receptor modulator is ketanserin.

In some embodiments, the psychedelic is R-methylone, and the serotonin receptor modulator is ketanserin.

In some embodiments, the psychedelic is S-methylone, and the serotonin receptor modulator is ketanserin.

In some embodiments, the psychedelic is MDEA, and the serotonin receptor modulator is ketanserin.

In some embodiments, the psychedelic is S-MDEA, and the serotonin receptor modulator is ketanserin.

In some embodiments, the psychedelic is N-Ethyl-2-(5-Fluoro-1H-Indol-3YL)-N-Methylethan-1-Amine, and the serotonin receptor modulator is ketanserin.

In some embodiments, the psychedelic is 4-OH-DiPT hemi-glutarate, and the serotonin receptor modulator is ketanserin.

In some embodiments, the psychedelic is 5,6-Dimethoxy-2-Aminoindane, and the serotonin receptor modulator is ketanserin.

In some embodiments, the psychedelic is 5-Methoxy-2-Aminoindane, and the serotonin receptor modulator is ketanserin.

In some embodiments, the psychedelic is 2-Br-LSD, and the serotonin receptor modulator is ketanserin.

In some embodiments, the psychedelic is MDAI, and the serotonin receptor modulator is ketanserin.

In some embodiments, the psychedelic is psilocybin, and the serotonin receptor modulator is ritanserin.

In some embodiments, the psychedelic is LSD, and the serotonin receptor modulator is ritanserin.

In some embodiments, the psychedelic is DMT, and the serotonin receptor modulator is ritanserin.

In some embodiments, the psychedelic is 5-MeO-DMT, and the serotonin receptor modulator is ritanserin.

In some embodiments, the psychedelic is 4-Acetoxy-DMT, and the serotonin receptor modulator is ritanserin.

In some embodiments, the psychedelic is ALD-52, and the serotonin receptor modulator is ritanserin.

In some embodiments, the psychedelic is 1P-LSD, and the serotonin receptor modulator is ritanserin.

In some embodiments, the psychedelic is 2C-B, and the serotonin receptor modulator is ritanserin.

In some embodiments, the psychedelic is ibogaine, and the serotonin receptor modulator is ritanserin.

In some embodiments, the psychedelic is MDMA, and the serotonin receptor modulator is ritanserin.

In some embodiments, the psychedelic is DOM, and the serotonin receptor modulator is ritanserin.

In some embodiments, the psychedelic is mescaline, and the serotonin receptor modulator is ritanserin.

In some embodiments, the psychedelic is S-MDMA, and the serotonin receptor modulator is ritanserin.

In some embodiments, the psychedelic is R-MDMA, and the serotonin receptor modulator is ritanserin.

In some embodiments, the psychedelic is MBDB, and the serotonin receptor modulator is ritanserin.

In some embodiments, the psychedelic is methylone, and the serotonin receptor modulator is ritanserin.

In some embodiments, the psychedelic is R-methylone, and the serotonin receptor modulator is ritanserin.

In some embodiments, the psychedelic is S-methylone, and the serotonin receptor modulator is ritanserin.

In some embodiments, the psychedelic is MDEA, and the serotonin receptor modulator is ritanserin.

In some embodiments, the psychedelic is S-MDEA, and the serotonin receptor modulator is ritanserin.

In some embodiments, the psychedelic is N-Ethyl-2-(5-Fluoro-1H-Indol-3YL)-N-Methylethan-1-Amine, and the serotonin receptor modulator is ritanserin.

In some embodiments, the psychedelic is 4-OH-DiPT hemi-glutarate, and the serotonin receptor modulator is ritanserin.

In some embodiments, the psychedelic is 5,6-Dimethoxy-2-Aminoindane, and the serotonin receptor modulator is ritanserin.

In some embodiments, the psychedelic is 5-Methoxy-2-Aminoindane, and the serotonin receptor modulator is ritanserin.

In some embodiments, the psychedelic is 2-Br-LSD, and the serotonin receptor modulator is ritanserin.

In some embodiments, the psychedelic is MDAI, and the serotonin receptor modulator is ritanserin.

In some embodiments, the psychedelic is psilocybin, and the serotonin receptor modulator is pimavanserin.

In some embodiments, the psychedelic is LSD, and the serotonin receptor modulator is pimavanserin.

In some embodiments, the psychedelic is DMT, and the serotonin receptor modulator is pimavanserin.

In some embodiments, the psychedelic is 5-MeO-DMT, and the serotonin receptor modulator is pimavanserin.

In some embodiments, the psychedelic is 4-Acetoxy-DMT, and the serotonin receptor modulator is pimavanserin.

In some embodiments, the psychedelic is ALD-52, and the serotonin receptor modulator is pimavanserin.

In some embodiments, the psychedelic is 1P-LSD, and the serotonin receptor modulator is pimavanserin.

In some embodiments, the psychedelic is 2C-B, and the serotonin receptor modulator is pimavanserin.

In some embodiments, the psychedelic is ibogaine, and the serotonin receptor modulator is pimavanserin.

In some embodiments, the psychedelic is MDMA, and the serotonin receptor modulator is pimavanserin.

In some embodiments, the psychedelic is DOM, and the serotonin receptor modulator is pimavanserin.

In some embodiments, the psychedelic is mescaline, and the serotonin receptor modulator is pimavanserin.

In some embodiments, the psychedelic is S-MDMA, and the serotonin receptor modulator is pimavanserin.

In some embodiments, the psychedelic is R-MDMA, and the serotonin receptor modulator is pimavanserin.

In some embodiments, the psychedelic is MBDB, and the serotonin receptor modulator is pimavanserin.

In some embodiments, the psychedelic is methylone, and the serotonin receptor modulator is pimavanserin.

In some embodiments, the psychedelic is R-methylone, and the serotonin receptor modulator is pimavanserin.

In some embodiments, the psychedelic is S-methylone, and the serotonin receptor modulator is pimavanserin.

In some embodiments, the psychedelic is MDEA, and the serotonin receptor modulator is pimavanserin.

In some embodiments, the psychedelic is S-MDEA, and the serotonin receptor modulator is pimavanserin.

In some embodiments, the psychedelic is N-Ethyl-2-(5-Fluoro-1H-Indol-3YL)-N-Methylethan-1-Amine, and the serotonin receptor modulator is pimavanserin.

In some embodiments, the psychedelic is 4-OH-DiPT hemi-glutarate, and the serotonin receptor modulator is pimavanserin.

In some embodiments, the psychedelic is 5,6-Dimethoxy-2-Aminoindane, and the serotonin receptor modulator is pimavanserin.

In some embodiments, the psychedelic is 5-Methoxy-2-Aminoindane, and the serotonin receptor modulator is pimavanserin.

In some embodiments, the psychedelic is 2-Br-LSD, and the serotonin receptor modulator is pimavanserin.

In some embodiments, the psychedelic is MDAI, and the serotonin receptor modulator is pimavanserin.

In some embodiments, the psychedelic is psilocybin, and the serotonin receptor modulator is nelotanserin.

In some embodiments, the psychedelic is LSD, and the serotonin receptor modulator is nelotanserin.

In some embodiments, the psychedelic is DMT, and the serotonin receptor modulator is nelotanserin.

In some embodiments, the psychedelic is 5-MeO-DMT, and the serotonin receptor modulator is nelotanserin.

In some embodiments, the psychedelic is 4-Acetoxy-DMT, and the serotonin receptor modulator is nelotanserin.

In some embodiments, the psychedelic is ALD-52, and the serotonin receptor modulator is nelotanserin.

In some embodiments, the psychedelic is 1P-LSD, and the serotonin receptor modulator is nelotanserin.

In some embodiments, the psychedelic is 2C-B, and the serotonin receptor modulator is nelotanserin.

In some embodiments, the psychedelic is ibogaine, and the serotonin receptor modulator is nelotanserin.

In some embodiments, the psychedelic is MDMA, and the serotonin receptor modulator is nelotanserin.

In some embodiments, the psychedelic is DOM, and the serotonin receptor modulator is nelotanserin.

In some embodiments, the psychedelic is mescaline, and the serotonin receptor modulator is nelotanserin.

In some embodiments, the psychedelic is S-MDMA, and the serotonin receptor modulator is nelotanserin.

In some embodiments, the psychedelic is R-MDMA, and the serotonin receptor modulator is nelotanserin.

In some embodiments, the psychedelic is MBDB, and the serotonin receptor modulator is nelotanserin.

In some embodiments, the psychedelic is methylone, and the serotonin receptor modulator is nelotanserin.

In some embodiments, the psychedelic is R-methylone, and the serotonin receptor modulator is nelotanserin.

In some embodiments, the psychedelic is S-methylone, and the serotonin receptor modulator is nelotanserin.

In some embodiments, the psychedelic is MDEA, and the serotonin receptor modulator is nelotanserin.

In some embodiments, the psychedelic is S-MDEA, and the serotonin receptor modulator is nelotanserin.

In some embodiments, the psychedelic is N-Ethyl-2-(5-Fluoro-1H-Indol-3YL)-N-Methylethan-1-Amine, and the serotonin receptor modulator is nelotanserin.

In some embodiments, the psychedelic is 4-OH-DiPT hemi-glutarate, and the serotonin receptor modulator is nelotanserin.

In some embodiments, the psychedelic is 5,6-Dimethoxy-2-Aminoindane, and the serotonin receptor modulator is nelotanserin.

In some embodiments, the psychedelic is 5-Methoxy-2-Aminoindane, and the serotonin receptor modulator is nelotanserin.

In some embodiments, the psychedelic is 2-Br-LSD, and the serotonin receptor modulator is nelotanserin.

In some embodiments, the psychedelic is MDAI, and the serotonin receptor modulator is nelotanserin.

In some embodiments, the psychedelic is psilocybin, and the serotonin receptor modulator is pruvanserin.

In some embodiments, the psychedelic is LSD, and the serotonin receptor modulator is pruvanserin.

In some embodiments, the psychedelic is DMT, and the serotonin receptor modulator is pruvanserin.

In some embodiments, the psychedelic is 5-MeO-DMT, and the serotonin receptor modulator is pruvanserin.

In some embodiments, the psychedelic is 4-Acetoxy-DMT, and the serotonin receptor modulator is pruvanserin.

In some embodiments, the psychedelic is ALD-52, and the serotonin receptor modulator is pruvanserin.

In some embodiments, the psychedelic is 1P-LSD, and the serotonin receptor modulator is pruvanserin.

In some embodiments, the psychedelic is 2C-B, and the serotonin receptor modulator is pruvanserin.

In some embodiments, the psychedelic is ibogaine, and the serotonin receptor modulator is pruvanserin.

In some embodiments, the psychedelic is MDMA, and the serotonin receptor modulator is pruvanserin.

In some embodiments, the psychedelic is DOM, and the serotonin receptor modulator is pruvanserin.

In some embodiments, the psychedelic is mescaline, and the serotonin receptor modulator is pruvanserin.

In some embodiments, the psychedelic is S-MDMA, and the serotonin receptor modulator is pruvanserin.

In some embodiments, the psychedelic is R-MDMA, and the serotonin receptor modulator is pruvanserin.

In some embodiments, the psychedelic is MBDB, and the serotonin receptor modulator is pruvanserin.

In some embodiments, the psychedelic is methylone, and the serotonin receptor modulator is pruvanserin.

In some embodiments, the psychedelic is R-methylone, and the serotonin receptor modulator is pruvanserin.

In some embodiments, the psychedelic is S-methylone, and the serotonin receptor modulator is pruvanserin.

In some embodiments, the psychedelic is MDEA, and the serotonin receptor modulator is pruvanserin.

In some embodiments, the psychedelic is S-MDEA, and the serotonin receptor modulator is pruvanserin.

In some embodiments, the psychedelic is N-Ethyl-2-(5-Fluoro-1H-Indol-3YL)-N-Methylethan-1-Amine, and the serotonin receptor modulator is pruvanserin.

In some embodiments, the psychedelic is 4-OH-DiPT hemi-glutarate, and the serotonin receptor modulator is pruvanserin.

In some embodiments, the psychedelic is 5,6-Dimethoxy-2-Aminoindane, and the serotonin receptor modulator is pruvanserin.

In some embodiments, the psychedelic is 5-Methoxy-2-Aminoindane, and the serotonin receptor modulator is pruvanserin.

In some embodiments, the psychedelic is 2-Br-LSD, and the serotonin receptor modulator is pruvanserin.

In some embodiments, the psychedelic is MDAI, and the serotonin receptor modulator is pruvanserin.

In some embodiments, the psychedelic is psilocybin, and the serotonin receptor modulator is flibanserin.

In some embodiments, the psychedelic is LSD, and the serotonin receptor modulator is flibanserin.

In some embodiments, the psychedelic is DMT, and the serotonin receptor modulator is flibanserin.

In some embodiments, the psychedelic is 5-MeO-DMT, and the serotonin receptor modulator is flibanserin.

In some embodiments, the psychedelic is 4-Acetoxy-DMT, and the serotonin receptor modulator is flibanserin.

In some embodiments, the psychedelic is ALD-52, and the serotonin receptor modulator is flibanserin.

In some embodiments, the psychedelic is 1P-LSD, and the serotonin receptor modulator is flibanserin.

In some embodiments, the psychedelic is 2C-B, and the serotonin receptor modulator is flibanserin.

In some embodiments, the psychedelic is ibogaine, and the serotonin receptor modulator is flibanserin.

In some embodiments, the psychedelic is MDMA, and the serotonin receptor modulator is flibanserin.

In some embodiments, the psychedelic is DOM, and the serotonin receptor modulator is flibanserin.

In some embodiments, the psychedelic is mescaline, and the serotonin receptor modulator is flibanserin.

In some embodiments, the psychedelic is S-MDMA, and the serotonin receptor modulator is flibanserin.

In some embodiments, the psychedelic is R-MDMA, and the serotonin receptor modulator is flibanserin.

In some embodiments, the psychedelic is MBDB, and the serotonin receptor modulator is flibanserin.

In some embodiments, the psychedelic is methylone, and the serotonin receptor modulator is flibanserin.

In some embodiments, the psychedelic is R-methylone, and the serotonin receptor modulator is flibanserin.

In some embodiments, the psychedelic is S-methylone, and the serotonin receptor modulator is flibanserin.

In some embodiments, the psychedelic is MDEA, and the serotonin receptor modulator is flibanserin.

In some embodiments, the psychedelic is S-MDEA, and the serotonin receptor modulator is flibanserin.

In some embodiments, the psychedelic is N-Ethyl-2-(5-Fluoro-1H-Indol-3YL)-N-Methylethan-1-Amine, and the serotonin receptor modulator is flibanserin.

In some embodiments, the psychedelic is 4-OH-DiPT hemi-glutarate, and the serotonin receptor modulator is flibanserin.

In some embodiments, the psychedelic is 5,6-Dimethoxy-2-Aminoindane, and the serotonin receptor modulator is flibanserin.

In some embodiments, the psychedelic is 5-Methoxy-2-Aminoindane, and the serotonin receptor modulator is flibanserin.

In some embodiments, the psychedelic is 2-Br-LSD, and the serotonin receptor modulator is flibanserin.

In some embodiments, the psychedelic is MDAI, and the serotonin receptor modulator is flibanserin.

In some embodiments, the psychedelic is psilocybin, and the serotonin receptor modulator is olanzapine.

In some embodiments, the psychedelic is LSD, and the serotonin receptor modulator is olanzapine.

In some embodiments, the psychedelic is DMT, and the serotonin receptor modulator is olanzapine.

In some embodiments, the psychedelic is 5-MeO-DMT, and the serotonin receptor modulator is olanzapine.

In some embodiments, the psychedelic is 4-Acetoxy-DMT, and the serotonin receptor modulator is olanzapine.

In some embodiments, the psychedelic is ALD-52, and the serotonin receptor modulator is olanzapine.

In some embodiments, the psychedelic is 1P-LSD, and the serotonin receptor modulator is olanzapine.

In some embodiments, the psychedelic is 2C-B, and the serotonin receptor modulator is olanzapine.

In some embodiments, the psychedelic is ibogaine, and the serotonin receptor modulator is olanzapine.

In some embodiments, the psychedelic is MDMA, and the serotonin receptor modulator is olanzapine.

In some embodiments, the psychedelic is DOM, and the serotonin receptor modulator is olanzapine.

In some embodiments, the psychedelic is mescaline, and the serotonin receptor modulator is olanzapine.

In some embodiments, the psychedelic is S-MDMA, and the serotonin receptor modulator is olanzapine.

In some embodiments, the psychedelic is R-MDMA, and the serotonin receptor modulator is olanzapine.

In some embodiments, the psychedelic is MBDB, and the serotonin receptor modulator is olanzapine.

In some embodiments, the psychedelic is methylone, and the serotonin receptor modulator is olanzapine.

In some embodiments, the psychedelic is R-methylone, and the serotonin receptor modulator is olanzapine.

In some embodiments, the psychedelic is S-methylone, and the serotonin receptor modulator is olanzapine.

In some embodiments, the psychedelic is MDEA, and the serotonin receptor modulator is olanzapine.

In some embodiments, the psychedelic is S-MDEA, and the serotonin receptor modulator is olanzapine.

In some embodiments, the psychedelic is N-Ethyl-2-(5-Fluoro-1H-Indol-3YL)-N-Methylethan-1-Amine, and the serotonin receptor modulator is olanzapine.

In some embodiments, the psychedelic is 4-OH-DiPT hemi-glutarate, and the serotonin receptor modulator is olanzapine.

In some embodiments, the psychedelic is 5,6-Dimethoxy-2-Aminoindane, and the serotonin receptor modulator is olanzapine.

In some embodiments, the psychedelic is 5-Methoxy-2-Aminoindane, and the serotonin receptor modulator is olanzapine.

In some embodiments, the psychedelic is 2-Br-LSD, and the serotonin receptor modulator is olanzapine.

In some embodiments, the psychedelic is MDAI, and the serotonin receptor modulator is olanzapine.

In some embodiments, the psychedelic is psilocybin, and the serotonin receptor modulator is quetiapine.

In some embodiments, the psychedelic is LSD, and the serotonin receptor modulator is quetiapine.

In some embodiments, the psychedelic is DMT, and the serotonin receptor modulator is quetiapine.

In some embodiments, the psychedelic is 5-MeO-DMT, and the serotonin receptor modulator is quetiapine.

In some embodiments, the psychedelic is 4-Acetoxy-DMT, and the serotonin receptor modulator is quetiapine.

In some embodiments, the psychedelic is ALD-52, and the serotonin receptor modulator is quetiapine.

In some embodiments, the psychedelic is 1P-LSD, and the serotonin receptor modulator is quetiapine.

In some embodiments, the psychedelic is 2C-B, and the serotonin receptor modulator is quetiapine.

In some embodiments, the psychedelic is ibogaine, and the serotonin receptor modulator is quetiapine.

In some embodiments, the psychedelic is MDMA, and the serotonin receptor modulator is quetiapine.

In some embodiments, the psychedelic is DOM, and the serotonin receptor modulator is quetiapine.

In some embodiments, the psychedelic is mescaline, and the serotonin receptor modulator is quetiapine.

In some embodiments, the psychedelic is S-MDMA, and the serotonin receptor modulator is quetiapine.

In some embodiments, the psychedelic is R-MDMA, and the serotonin receptor modulator is quetiapine.

In some embodiments, the psychedelic is MBDB, and the serotonin receptor modulator is quetiapine.

In some embodiments, the psychedelic is methylone, and the serotonin receptor modulator is quetiapine.

In some embodiments, the psychedelic is R-methylone, and the serotonin receptor modulator is quetiapine.

In some embodiments, the psychedelic is S-methylone, and the serotonin receptor modulator is quetiapine.

In some embodiments, the psychedelic is MDEA, and the serotonin receptor modulator is quetiapine.

In some embodiments, the psychedelic is S-MDEA, and the serotonin receptor modulator is quetiapine.

In some embodiments, the psychedelic is N-Ethyl-2-(5-Fluoro-1H-Indol-3YL)-N-Methylethan-1-Amine, and the serotonin receptor modulator is quetiapine.

In some embodiments, the psychedelic is 4-OH-DiPT hemi-glutarate, and the serotonin receptor modulator is quetiapine.

In some embodiments, the psychedelic is 5,6-Dimethoxy-2-Aminoindane, and the serotonin receptor modulator is quetiapine.

In some embodiments, the psychedelic is 5-Methoxy-2-Aminoindane, and the serotonin receptor modulator is quetiapine.

In some embodiments, the psychedelic is 2-Br-LSD, and the serotonin receptor modulator is quetiapine.

In some embodiments, the psychedelic is MDAI, and the serotonin receptor modulator is quetiapine.

In some embodiments, the psychedelic is psilocybin, and the serotonin receptor modulator is risperidone.

In some embodiments, the psychedelic is LSD, and the serotonin receptor modulator is risperidone.

In some embodiments, the psychedelic is DMT, and the serotonin receptor modulator is risperidone.

In some embodiments, the psychedelic is 5-MeO-DMT, and the serotonin receptor modulator is risperidone.

In some embodiments, the psychedelic is 4-Acetoxy-DMT, and the serotonin receptor modulator is risperidone.

In some embodiments, the psychedelic is ALD-52, and the serotonin receptor modulator is risperidone.

In some embodiments, the psychedelic is 1P-LSD, and the serotonin receptor modulator is risperidone.

In some embodiments, the psychedelic is 2C-B, and the serotonin receptor modulator is risperidone.

In some embodiments, the psychedelic is ibogaine, and the serotonin receptor modulator is risperidone.

In some embodiments, the psychedelic is MDMA, and the serotonin receptor modulator is risperidone.

In some embodiments, the psychedelic is DOM, and the serotonin receptor modulator is risperidone.

In some embodiments, the psychedelic is mescaline, and the serotonin receptor modulator is risperidone.

In some embodiments, the psychedelic is S-MDMA, and the serotonin receptor modulator is risperidone.

In some embodiments, the psychedelic is R-MDMA, and the serotonin receptor modulator is risperidone.

In some embodiments, the psychedelic is MBDB, and the serotonin receptor modulator is risperidone.

In some embodiments, the psychedelic is methylone, and the serotonin receptor modulator is risperidone.

In some embodiments, the psychedelic is R-methylone, and the serotonin receptor modulator is risperidone.

In some embodiments, the psychedelic is S-methylone, and the serotonin receptor modulator is risperidone.

In some embodiments, the psychedelic is MDEA, and the serotonin receptor modulator is risperidone.

In some embodiments, the psychedelic is S-MDEA, and the serotonin receptor modulator is risperidone.

In some embodiments, the psychedelic is N-Ethyl-2-(5-Fluoro-1H-Indol-3YL)-N-Methylethan-1-Amine, and the serotonin receptor modulator is risperidone.

In some embodiments, the psychedelic is 4-OH-DiPT hemi-glutarate, and the serotonin receptor modulator is risperidone.

In some embodiments, the psychedelic is 5,6-Dimethoxy-2-Aminoindane, and the serotonin receptor modulator is risperidone.

In some embodiments, the psychedelic is 5-Methoxy-2-Aminoindane, and the serotonin receptor modulator is risperidone.

In some embodiments, the psychedelic is 2-Br-LSD, and the serotonin receptor modulator is risperidone.

In some embodiments, the psychedelic is MDAI, and the serotonin receptor modulator is risperidone.

In some embodiments, the psychedelic is selected from the group consisting of psilocybin, 4-Acetoxy-DMT, LSD, ALD-52, 1P-LSD, DMT, and 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is flibanserin.

In some embodiments, the psychedelic is selected from the group consisting of psilocybin, 4-Acetoxy-DMT, LSD, ALD-52, 1P-LSD, DMT, and 5-MeO-DMT, 2C-B, ibogaine, MDMA, DOM, mescaline, R-MDMA, S-MDMA, MBDB, Methylone, R-methylone, S-methylone, MDEA, S-MDEA, N-Ethyl-2-(5-Fluoro-1H-Indol-3YL)-N-Methylethan-1-Amine, 4-OH-DiPT hemi-glutarate, 5,6-Dimethoxy-2-Aminoindane, 5-Methoxy-2-Aminoindane, 2-Br-LSD, and MDAI, and the serotonin receptor modulator is selected from the group consisting of eplivanserin, volinanserin, ketanserin, ritanserin, pimavanserin, nelotanserin, pruvanserin, flibanserin, olanzapine, quetiapine, and risperidone.

In some embodiments, the psychedelic is selected from the group consisting of psilocybin, LSD, DMT, and 5-MeO-DMT and the serotonin receptor modulator is selected from the group consisting of eplivanserin, volinanserin, pimavanserin, nelotanserin, and pruvanserin, flibanserin, olanzapine, quetiapine, and risperidone.

In some embodiments, the psychedelic is selected from the group consisting of psilocybin, LSD, DMT, and 5-MeO-DMT5-MeO-DMT and the serotonin receptor modulator is eplivanserin or volinanserin.

In some embodiments, the psychedelic is selected from the group consisting of psilocybin, LSD, DMT, and 5-MeO-DMT and the serotonin receptor modulator is eplivanserin.

In some embodiments, the psychedelic is selected from the group consisting of psilocybin, LSD, DMT, and 5-MeO-DMT and the serotonin receptor modulator is volinanserin.

In some embodiments, the psychedelic is psilocybin and the serotonin receptor modulator is eplivanserin or volinanserin. In some embodiments, the psychedelic is LSD, and the serotonin receptor modulator is eplivanserin or volinanserin. In some embodiments, the psychedelic is DMT, and the serotonin receptor modulator is eplivanserin or volinanserin. In some embodiments, the psychedelic is 5-MeO-DMT and the serotonin receptor modulator is eplivanserin or volinanserin.

In some embodiments, the psychedelic is selected from the group consisting of psilocybin, LSD, DMT, and 5-MeO-DMT and the serotonin receptor modulator is selected from the group consisting of pimavanserin, nelotanserin, and pruvanserin.

In some embodiments, the psychedelic is psilocybin, and the serotonin receptor modulator is selected from the group consisting of pimavanserin, nelotanserin, and pruvanserin.

In some embodiments, the psychedelic is LSD, and the serotonin receptor modulator is selected from the group consisting of pimavanserin, nelotanserin, and pruvanserin.

In some embodiments, the psychedelic is DMT, and the serotonin receptor modulator is selected from the group consisting of pimavanserin, nelotanserin, and pruvanserin.

In some embodiments, the psychedelic is 5-MeO-DMT and the serotonin receptor modulator is selected from the group consisting of pimavanserin, nelotanserin, and pruvanserin.

In some embodiments, the psychedelic is psilocybin, and the serotonin receptor modulator is selected from the group consisting of pimavanserin, and nelotanserin.

In some embodiments, the psychedelic is LSD, and the serotonin receptor modulator is selected from the group consisting of pimavanserin, and nelotanserin.

In some embodiments, the psychedelic is DMT, and the serotonin receptor modulator is selected from the group consisting of pimavanserin, and nelotanserin.

In some embodiments, the psychedelic is 5-MeO-DMT and the serotonin receptor modulator is selected from the group consisting of pimavanserin, and nelotanserin.

In some embodiments, the serotonin receptor modulator is eplivanserin and the psychedelic is psilocybin. In some embodiments, the serotonin receptor modulator is volinanserin and the psychedelic is psilocybin. In some embodiments, the serotonin receptor modulator is ketanserin and the psychedelic is psilocybin. In some embodiments, the serotonin receptor modulator is ritanserin and the psychedelic is psilocybin. In some embodiments, the serotonin receptor modulator is pimavanserin and the psychedelic is psilocybin. In some embodiments, the serotonin receptor modulator is nelotanserin and the psychedelic is psilocybin. In some embodiments, the serotonin receptor modulator is pruvanserin and the psychedelic is psilocybin.

In some embodiments, the serotonin receptor modulator is eplivanserin and the psychedelic is LSD. In some embodiments, the serotonin receptor modulator is volinanserin and the psychedelic is LSD. In some embodiments, the serotonin receptor modulator is ketanserin and the psychedelic is LSD. In some embodiments, the serotonin receptor modulator is ritanserin and the psychedelic is LSD. In some embodiments, the serotonin receptor modulator is pimavanserin and the psychedelic is LSD. In some embodiments, the serotonin receptor modulator is nelotanserin and the psychedelic is LSD. In some embodiments, the serotonin receptor modulator is pruvanserin and the psychedelic is LSD.

In some embodiments, the serotonin receptor modulator is eplivanserin and the psychedelic is DMT. In some embodiments, the serotonin receptor modulator is volinanserin and the psychedelic is DMT. In some embodiments, the serotonin receptor modulator is ketanserin and the psychedelic is DMT. In some embodiments, the serotonin receptor modulator is ritanserin and the psychedelic is DMT. In some embodiments, the serotonin receptor modulator is pimavanserin and the psychedelic is DMT. In some embodiments, the serotonin receptor modulator is nelotanserin and the psychedelic is DMT. In some embodiments, the serotonin receptor modulator is pruvanserin and the psychedelic is DMT.

In some embodiments, the serotonin receptor modulator is eplivanserin and the psychedelic is 5-MeO-DMT. In some embodiments, the serotonin receptor modulator is volinanserin and the psychedelic is 5-MeO-DMT. In some embodiments, the serotonin receptor modulator is ketanserin and the psychedelic is 5-MeO-DMT. In some embodiments, the serotonin receptor modulator is ritanserin and the psychedelic is 5-MeO-DMT. In some embodiments, the serotonin receptor modulator is pimavanserin and the psychedelic is 5-MeO-DMT. In some embodiments, the serotonin receptor modulator is nelotanserin and the psychedelic is 5-MeO-DMT. In some embodiments, the serotonin receptor modulator is pruvanserin and the psychedelic is 5-MeO-DMT.

In some embodiments, each of the combinations of psychedelics and serotonin receptor modulators described herein are contemplated at each of the following dosages and dose ranges described herein. In some embodiments, each of the combinations of psychedelics and serotonin receptor modulators described herein are contemplated at each of the following administration time periods described herein. In some embodiments, each of the combinations of psychedelics and serotonin receptor modulators described herein are contemplated at each of the following dosages and dose ranges and each of the following administration time periods described herein.

Described herein, in some embodiments, are compositions comprising a psychedelic and a serotonin receptor modulator, wherein the psychedelic (e.g., psilocybin, psilocin, 0-acetyl psilocin, LSD, 1-acetyl LSD) is provided at varying doses. In some embodiments, the psychedelic is provided in a range about 10 milligrams (mg) to 400 mg, about 10 mg to about 300 mg, about 10 mg to about 200 mg, about 10 mg to about 100 mg, about 10 mg to about 80 mg, about 10 mg to about 70 mg, about 10 mg to about 60 mg, about 10 mg to about 50 mg, about 10 mg to about 40 mg, about 20 mg to 400 mg, about 20 mg to about 300 mg, about 20 mg to about 200 mg, about 20 mg to about 100 mg, about 20 mg to about 80 mg, about 20 mg to about 70 mg, about 20 mg to about 60 mg, about 20 mg to about 50 mg, about 20 mg to about 40 mg, about 50 mg to 400 mg, about 50 mg to about 300 mg, about 50 mg to about 200 mg, about 50 mg to about 100 mg, about 50 mg to about 80 mg, about 50 mg to about 70 mg, or about 50 mg to about 60 mg. In some embodiments, the psychedelic is provided at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, or about 500 mg. In some embodiments, the psychedelic is a phenethylamine or a tryptamine.

In some embodiments, the psychedelic is provided in a range about 10 micrograms (μg) to 400 μg, about 10 μg to about 300 μg, about 10 μg to about 200 μg, about 10 μg to about 100 μg, about 10 μg to about 80 μg, about 10 μg to about 70 μg, about 10 μg to about 60 μg, about 10 μg to about 50 μg, about 10 μg to about 40 μg, about 20 μg to 400 μg, about 20 μg to about 300 μg, about 20 μg to about 200 μg, about 20 μg to about 100 μg, about 20 μg to about 80 μg, about 20 μg to about 70 μg, about 20 μg to about 60 μg, about 20 μg to about 50 μg, about 20 μg to about 40 μg, about 50 μg to 400 μg, about 50 μg to about 300 μg, about 50 μg to about 200 μg, about 50 μg to about 100 μg, about 50 μg to about 80 μg, about 50 μg to about 70 μg, or about 50 μg to about 60 μg. In some embodiments, the psychedelic is provided at about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 240 μg, about 250 μg, about 260 μg, about 270 μg, about 275 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, 410 μg, about 420 μg, about 430 μg, about 440 μg, about 450 μg, about 460 μg, about 470 μg, about 480 μg, about 490 μg, or about 500 μg. In some embodiments, the psychedelic is LSD. In some embodiments, the LSD derivative is 1P-LSD, 1B-LSD, ETH-LAD, 1P-ETH-LAD, AL-LAD, LSZ, LSM-775, 1-(4-Bromofuro[2,3-f] [1]benzofuran-8-yl)propan-2-amine.

In some embodiments, the psychedelic is provided in a range about 100 milligrams (mg) to 4 grams (g), about 100 mg to about 3 g, about 100 mg to about 2 g, about 100 mg to about 1 g, about 100 mg to about 800 mg, about 100 mg to about 700 mg, about 100 mg to about 600 mg, about 100 mg to about 500 mg, about 100 mg to about 400 mg, about 200 mg to 4 g, about 200 mg to about 3 g, about 200 mg to about 2 g, about 200 mg to about 1 g, about 200 mg to about 800 mg, about 200 mg to about 700 mg, about 200 mg to about 600 mg, about 200 mg to about 500 mg, about 200 mg to about 400 mg, about 500 mg to 4 g, about 500 mg to about 3 g, about 500 mg to about 2 g, about 500 mg to about 1 g, about 500 mg to about 800 mg, about 500 mg to about 700 mg, or about 500 mg to about 600 mg. In some embodiments, the psychedelic is provided at about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 2 g, about 3 g, about 4 g, or about 5 g. In some embodiments, the psychedelic is mescaline. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine).

Described herein, in some embodiments, are compositions comprising a psychedelic and a serotonin receptor modulator, wherein the serotonin receptor modulator (e.g., ketanserin, pimavanserin) is provided at varying doses. In some embodiments, the serotonin receptor modulator is provided in a range about 10 mg to 400 mg, about 10 mg to about 300 mg, about 10 mg to about 200 mg, about 10 mg to about 100 mg, about 10 mg to about 80 mg, about 10 mg to about 70 mg, about 10 mg to about 60 mg, about 10 mg to about 50 mg, about 10 mg to about 40 mg, about 20 mg to 400 mg, about 20 mg to about 300 mg, about 20 mg to about 200 mg, about 20 mg to about 100 mg, about 20 mg to about 80 mg, about 20 mg to about 70 mg, about 20 mg to about 60 mg, about 20 mg to about 50 mg, about 20 mg to about 40 mg, about 50 mg to 400 mg, about 50 mg to about 300 mg, about 50 mg to about 200 mg, about 50 mg to about 100 mg, about 50 mg to about 80 mg, about 50 mg to about 70 mg, or about 50 mg to about 60 mg. In some embodiments, the serotonin receptor is provided at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 250 mg, about 260 mg, about 270 mg, about 275 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, or about 500 mg.

In some embodiments, the serotonin receptor modulator is selected from the group consisting of ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, and lumateperone.

In some embodiments, the serotonin receptor modulator is selected from the group consisting of MDL-11,939, eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, MDL-100,907, pimavanserin, nelotanserin, lorcaserin, flibanserin, and roluperiodone. In some embodiments, the serotonin receptor modulator is ketanserin.

In some embodiments, the serotonin receptor modulator is ketanserin.

In some embodiments, the serotonin receptor modulator is pimavanserin.

In some embodiments, the serotonin receptor modulator is eplivanserin.

In some embodiments, the serotonin receptor modulator is volinanserin.

In some embodiments, the serotonin receptor modulator is pruvanserin.

In some embodiments, the serotonin receptor modulator is nelotanserin.

In some embodiments, the serotonin receptor modulator is flibanserin.

In some embodiments, the serotonin receptor modulator is ritanserin.

In some embodiments, the serotonin receptor modulator is olanzapine.

In some embodiments, the serotonin receptor modulator is quetiapine.

In some embodiments, the serotonin receptor modulator is risperidone.

In some embodiments, the serotonin receptor modulator is eplivanserin and the psychedelic is psilocybin, wherein the eplivanserin is provided between about 1 mg to about 40 mg, or about 5 mg to about 10 mg, and the psilocybin is provided between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is volinanserin and the psychedelic is psilocybin, wherein the volinanserin is provided in about 1 mg to about 60 mg, or about 5 mg to about 20 mg, and the psilocybin is provided between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is ketanserin and the psychedelic is psilocybin, wherein the ketanserin is provided in about 10 mg to about 80 mg, about 30 mg to about 50 mg, or about 40 mg and the psilocybin is provided between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is ritanserin and the psychedelic is psilocybin, wherein the ritanserin is provided in about 1 mg to about 40 mg, or about 2.5 mg to about 10 mg, and the psilocybin is provided between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is pimavanserin and the psychedelic is psilocybin, wherein the pimavanserin is provided in about 1 mg to about 60 mg, or about 17 mg to about 34 mg, and the psilocybin is provided between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is nelotanserin and the psychedelic is psilocybin, wherein the nelotanserin is provided in about 1 mg to about 80 mg, or about 40 mg to about 80 mg, and the psilocybin is provided between about 10 mg to about 50 mg, about 25 mg to about 30 mg, about 35 mg or about 40 mg, about 25 mg, about 30 mg, about 35 mg, or about 40 mg.

In some embodiments, the serotonin receptor modulator is pruvanserin and the psychedelic is psilocybin, wherein the pruvanserin is provided in about 1 mg to about 40 mg, or about 3 mg to about 10 mg, and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is flibanserin and the psychedelic is psilocybin, wherein the flibanserin is present in about 10 mg to about 200 mg, or about 80 mg to about 120 mg, or about 100 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is olanzapine and the psychedelic is psilocybin, wherein the olanzapine is present in about 2.5 mg to about 30 mg, or about 5 mg or about 10 mg, or about 20 mg or about 25 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is an extended-release form of olanzapine (e.g., ZYPREXA RELPREVV) and the psychedelic is psilocybin, wherein the extended-release form of olanzapine (e.g., ZYPREXA RELPREVV) is present in about 50 mg to about 450 mg, or about 150 mg or about 210 mg, or about 300 mg or about 405 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is quetiapine and the psychedelic is psilocybin, wherein the quetiapine is present in about 25 mg to about 800 mg, or about 50 mg to about 100 mg, or about 150 mg or about 200 mg or about 250 mg or about 300 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is an extended-release form of quetiapine and the psychedelic is psilocybin, wherein the extended-release form of quetiapine is present in about 50 mg to about 300 mg, or about 50 mg or about 100 mg or about 200 mg, or about 300 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is risperidone and the psychedelic is psilocybin, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is an extended-release form of risperidone (e.g., RISPERDAL CONSTA) and the psychedelic is psilocybin, wherein the extended-release form of risperidone (e.g., RISPERDAL CONSTA) is present in about 12.5 mg, or about 25 mg, or about 37.5 mg, or about 50 mg and the psilocybin is present between about 10 mg to about 50 mg, about 25 mg to about 30 mg, about 35 mg to about 40 mg, about 25 mg, about 30 mg, about 35 mg, or about 40 mg.

In some embodiments, the serotonin receptor modulator is eplivanserin and the psychedelic is o-acetyl-psilocin (4-Acetoxy-DMT), wherein the eplivanserin is present in about 1 mg to about 40 mg, or about 5 mg to about 10 mg, and the 4-Acetoxy-DMT is present between about 5 mg to about 50 mg, about 10 mg to about 25 mg, about 30 mg, about 35 mg to about 40 mg.

In some embodiments, the serotonin receptor modulator is volinanserin and the psychedelic is 4-Acetoxy-DMT, wherein the volinanserin is present in about 1 mg to about 60 mg, or about 5 mg to about 20 mg, and the 4-Acetoxy-DMT is present between about 5 mg to about 50 mg, about 10 mg to about 25 mg, about 30 mg, about 35 mg, or about 40 mg.

In some embodiments, the serotonin receptor modulator is ketanserin and the psychedelic is 4-Acetoxy-DMT, wherein the ketanserin is present in about 10 mg to about 80 mg, about 30 mg to about 50 mg, or about 40 mg and the 4-Acetoxy-DMT is present between about 5 mg to about 50 mg, about 10 mg to about 25 mg, about 30 mg, or about 35 mg to about 40 mg.

In some embodiments, the serotonin receptor modulator is ritanserin and the psychedelic is 4-Acetoxy-DMT, wherein the ritanserin is present in about 1 mg to about 40 mg, or about 2.5 mg to about 10 mg, and the 4-Acetoxy-DMT is present between about 5 mg to about 50 mg, about 10 mg to about 25 mg, about 30 mg, about 35 mg, or about 40 mg.

In some embodiments, the serotonin receptor modulator is pimavanserin and the psychedelic is 4-Acetoxy-DMT, wherein the pimavanserin is present in about 1 mg to about 60 mg, or about 17 mg to about 34 mg, and the 4-Acetoxy-DMT is present between about 5 mg to about 50 mg, about 10 mg to about 25 mg, about 30 mg, about 35 mg, or about 40 mg.

In some embodiments, the serotonin receptor modulator is nelotanserin and the psychedelic is 4-Acetoxy-DMT, wherein the nelotanserin is present in about 1 mg to about 80 mg, or about 40 mg to about 80 mg, and the 4-Acetoxy-DMT is present between about 5 mg to about 50 mg, about 10 mg to about 25 mg, about 30 mg, about 35 mg, or about 40 mg.

In some embodiments, the serotonin receptor modulator is pruvanserin and the psychedelic is 4-Acetoxy-DMT, wherein the pruvanserin is present in about 1 mg to about 40 mg, or about 3 mg to about 10 mg, and the 4-Acetoxy-DMT is present between about 5 mg to about 50 mg, about 10 mg to about 25 mg, about 30 mg, about 35 mg, or about 40 mg.

In some embodiments, the serotonin receptor modulator is flibanserin and the psychedelic is 4-Acetoxy-DMT, wherein the flibanserin is present in about 10 mg to about 200 mg, or about 80 mg to about 120 mg, or about 100 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is olanzapine and the psychedelic is 4-Acetoxy-DMT, wherein the olanzapine is present in about 2.5 mg to about 30 mg, or about 5 mg or about 10 mg, or about 20 mg or about 25 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is an extended-release form of olanzapine (e.g., ZYPREXA RELPREVV) and the psychedelic is 4-Acetoxy-DMT, wherein the extended-release form of olanzapine (e.g., ZYPREXA RELPREVV) is present in about 50 mg to about 450 mg, or about 150 mg or about 210 mg, or about 300 mg or about 405 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is quetiapine and the psychedelic is 4-Acetoxy-DMT, wherein the quetiapine is present in about 25 mg to about 800 mg, or about 50 mg to about 100 mg, or about 150 mg or about 200 mg or about 250 mg or about 300 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is an extended-release form of quetiapine and the psychedelic is 4-Acetoxy-DMT, wherein the extended-release form of quetiapine is present in about 50 mg to about 300 mg, or about 50 mg or about 100 mg or about 200 mg, or about 300 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is risperidone and the psychedelic is 4-Acetoxy-DMT, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is an extended-release form of risperidone (e.g., RISPERDAL CONSTA) and the psychedelic is 4-Acetoxy-DMT, wherein the extended-release form of risperidone (e.g., RISPERDAL CONSTA) is present in about 12.5 mg, or about 25 mg, or about 37.5 mg, or about 50 mg and the 4-Acetoxy-DMT is present between about 5 mg to about 50 mg, about 10 mg to about 25 mg, about 30 mg, about 35 mg, or about 40 mg.

In some embodiments, the serotonin receptor modulator is eplivanserin and the psychedelic is LSD, wherein the eplivanserin is present in about 1 mg to about 40 mg, or about 5 mg to about 10 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is volinanserin and the psychedelic is LSD, wherein the volinanserin is present in about 1 mg to about 60 mg, or about 5 mg to about 20 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is ketanserin and the psychedelic is LSD, wherein the ketanserin is present in about 10 mg to about 80 mg, about 30 mg to about 50 mg, or about 40 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is ritanserin and the psychedelic is LSD, wherein the ritanserin is present in about 1 mg to about 40 mg, or about 2.5 mg to about 10 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is pimavanserin and the psychedelic is LSD, wherein the pimavanserin is present in about 1 mg to about 60 mg, or about 17 mg to about 34 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is nelotanserin and the psychedelic is LSD, wherein the nelotanserin is present in about 1 mg to about 80 mg, or about 40 mg to about 80 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is pruvanserin and the psychedelic is LSD, wherein the pruvanserin is present in about 1 mg to about 40 mg, or about 3 mg to about 10 mg, and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is flibanserin and the psychedelic is LSD, wherein the flibanserin is present in about 10 mg to about 200 mg, or about 80 mg to about 120 mg, or about 100 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is olanzapine and the psychedelic is LSD, wherein the olanzapine is present in about 2.5 mg to about 30 mg, or about 5 mg or about 10 mg, or about 20 mg or about 25 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of olanzapine (e.g., ZYPREXA RELPREVV) and the psychedelic is LSD, wherein the extended-release form of olanzapine (e.g., ZYPREXA RELPREVV) is present in about 50 mg to about 450 mg, or about 150 mg or about 210 mg, or about 300 mg or about 405 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is quetiapine and the psychedelic is LSD, wherein the quetiapine is present in about 25 mg to about 800 mg, or about 50 mg to about 100 mg, or about 150 mg or about 200 mg or about 250 mg or about 300 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of quetiapine and the psychedelic is LSD, wherein the extended-release form of quetiapine is present in about 50 mg to about 300 mg, or about 50 mg or about 100 mg or about 200 mg, or about 300 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is risperidone and the psychedelic is LSD, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of risperidone (e.g., RISPERDAL CONSTA) and the psychedelic is LSD, wherein the extended-release form of risperidone (e.g., RISPERDAL CONSTA) is present in about 12.5 mg, or about 25 mg, or about 37.5 mg, or about 50 mg and the LSD is present between about 1 microgram to about 400 micrograms, about 50 micrograms to about 200 micrograms, about 100 micrograms, about 150 micrograms, or about 250 micrograms.

In some embodiments, the serotonin receptor modulator is eplivanserin and the psychedelic is ALD-52 (1-acetyl-LSD), wherein the eplivanserin is present in about 1 mg to about 40 mg, or about 5 mg to about 10 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is volinanserin and the psychedelic is ALD-52 (1-acetyl-LSD), wherein the volinanserin is present in about 1 mg to about 60 mg, or about 5 mg to about 20 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is ketanserin and the psychedelic is ALD-52, wherein the ketanserin is present in about 10 mg to about 80 mg, about 30 mg to about 50 mg, or about 40 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is ritanserin and the psychedelic is ALD-52, wherein the ritanserin is present in about 1 mg to about 40 mg, or about 2.5 mg to about 10 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is pimavanserin and the psychedelic is ALD-52, wherein the pimavanserin is present in about 1 mg to about 60 mg, or about 17 mg to about 34 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is nelotanserin and the psychedelic is ALD-52, wherein the nelotanserin is present in about 1 mg to about 80 mg, or about 40 mg to about 80 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is pruvanserin and the psychedelic is ALD-52, wherein the pruvanserin is present in about 1 mg to about 40 mg, or about 3 mg to about 10 mg, and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is flibanserin and the psychedelic is ALD-52, wherein the flibanserin is present in about 10 mg to about 200 mg, or about 80 mg to about 120 mg, or about 100 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is olanzapine and the psychedelic is ALD-52, wherein the olanzapine is present in about 2.5 mg to about 30 mg, or about 5 mg or about 10 mg, or about 20 mg or about 25 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of olanzapine (e.g., ZYPREXA RELPREVV) and the psychedelic is ALD-52, wherein the extended-release form of olanzapine (e.g., ZYPREXA RELPREVV) is present in about 50 mg to about 450 mg, or about 150 mg or about 210 mg, or about 300 mg or about 405 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is quetiapine and the psychedelic is ALD-52, wherein the quetiapine is present in about 25 mg to about 800 mg, or about 50 mg to about 100 mg, or about 150 mg or about 200 mg or about 250 mg or about 300 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of quetiapine and the psychedelic is ALD-52, wherein the extended-release form of quetiapine is present in about 50 mg to about 300 mg, or about 50 mg or about 100 mg or about 200 mg, or about 300 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is risperidone and the psychedelic is ALD-52, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of risperidone (e.g., RISPERDAL CONSTA) and the psychedelic is ALD-52, wherein the extended-release form of risperidone (e.g., RISPERDAL CONSTA) is present in about 12.5 mg, or about 25 mg, or about 37.5 mg, or about 50 mg and the ALD-52 is present between about 1 microgram to about 400 micrograms, about 50 micrograms to about 200 micrograms, about 100 micrograms, about 150 micrograms, or about 250 micrograms.

In some embodiments, the serotonin receptor modulator is eplivanserin and the psychedelic is 1P-LSD (1-propionyl-LSD), wherein the eplivanserin is present in about 1 mg to about 40 mg, or about 5 mg to about 10 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is volinanserin and the psychedelic is 1P-LSD, wherein the volinanserin is present in about 1 mg to about 60 mg, or about 5 mg to about 20 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is ketanserin and the psychedelic is 1P-LSD, wherein the ketanserin is present in about 10 mg to about 80 mg, about 30 mg to about 50 mg, or about 40 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is ritanserin and the psychedelic is 1P-LSD, wherein the ritanserin is present in about 1 mg to about 40 mg, or about 2.5 mg to about 10 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is pimavanserin and the psychedelic is 1P-LSD, wherein the pimavanserin is present in about 1 mg to about 60 mg, or about 17 mg to about 34 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is nelotanserin and the psychedelic is 1P-LSD, wherein the nelotanserin is present in about 1 mg to about 80 mg, or about 40 mg to about 80 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is pruvanserin and the psychedelic is 1P-LSD, wherein the pruvanserin is present in about 1 mg to about 40 mg, or about 3 mg to about 10 mg, and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is flibanserin and the psychedelic is 1P-LSD, wherein the flibanserin is present in about 10 mg to about 200 mg, or about 80 mg to about 120 mg, or about 100 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is olanzapine and the psychedelic is 1P-LSD, wherein the olanzapine is present in about 2.5 mg to about 30 mg, or about 5 mg or about 10 mg, or about 20 mg or about 25 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of olanzapine (e.g., ZYPREXA RELPREVV) and the psychedelic is 1P-LSD, wherein the extended-release form of olanzapine (e.g., ZYPREXA RELPREVV) is present in about 50 mg to about 450 mg, or about 150 mg or about 210 mg, or about 300 mg or about 405 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is quetiapine and the psychedelic is 1P-LSD, wherein the quetiapine is present in about 25 mg to about 800 mg, or about 50 mg to about 100 mg, or about 150 mg or about 200 mg or about 250 mg or about 300 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of quetiapine and the psychedelic is 1P-LSD, wherein the extended-release form of quetiapine is present in about 50 mg to about 300 mg, or about 50 mg or about 100 mg or about 200 mg, or about 300 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is risperidone and the psychedelic is 1P-LSD, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is an extended-release form of risperidone (e.g., RISPERDAL CONSTA) and the psychedelic is 1P-LSD, wherein the extended-release form of risperidone (e.g., RISPERDAL CONSTA) is present in about 12.5 mg, or about 25 mg, or about 37.5 mg, or about 50 mg and the 1P-LSD is present between about 1 microgram to about 400 micrograms, about 50 micrograms to about 200 micrograms, about 100 micrograms, about 150 micrograms, or about 250 micrograms.

In some embodiments, the serotonin receptor modulator is eplivanserin and the psychedelic is DMT, wherein the eplivanserin is present in about 1 mg to about 40 mg, or about 5 mg to about 10 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is volinanserin and the psychedelic is DMT, wherein the volinanserin is present in about 1 mg to about 60 mg, or about 5 mg to about 20 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is ketanserin and the psychedelic is DMT, wherein the ketanserin is present in about 10 mg to about 80 mg, about 30 mg to about 50 mg, or about 40 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is ritanserin and the psychedelic is DMT, wherein the ritanserin is present in about 1 mg to about 40 mg, or about 2.5 mg to about 10 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is pimavanserin and the psychedelic is DMT, wherein the pimavanserin is present in about 1 mg to about 60 mg, or about 17 mg to about 34 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is nelotanserin and the psychedelic is DMT, wherein the nelotanserin is present in about 1 mg to about 80 mg, or about 40 mg to about 80 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is pruvanserin and the psychedelic is DMT, wherein the pruvanserin is present in about 1 mg to about 40 mg, or about 3 mg to about 10 mg, and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is flibanserin and the psychedelic is DMT, wherein the flibanserin is present in about 10 mg to about 200 mg, or about 80 mg to about 120 mg, or about 100 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is olanzapine and the psychedelic is DMT, wherein the olanzapine is present in about 2.5 mg to about 30 mg, or about 5 mg or about 10 mg, or about 20 mg or about 25 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is an extended-release form of olanzapine (e.g., ZYPREXA RELPREVV) and the psychedelic is DMT, wherein the extended-release form of olanzapine (e.g., ZYPREXA RELPREVV) is present in about 50 mg to about 450 mg, or about 150 mg or about 210 mg, or about 300 mg or about 405 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is quetiapine and the psychedelic is DMT, wherein the quetiapine is present in about 25 mg to about 800 mg, or about 50 mg to about 100 mg, or about 150 mg or about 200 mg or about 250 mg or about 300 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is an extended-release form of quetiapine and the psychedelic is DMT, wherein the extended-release form of quetiapine is present in about 50 mg to about 300 mg, or about 50 mg or about 100 mg or about 200 mg, or about 300 mg and the DMT is present between about 1 mg and 60 mg, about 0.1 mg/kg to about 0.3 mg/kg, or about 0.1 mg/kg followed by 0.3 mg/kg

In some embodiments, the serotonin receptor modulator is risperidone and the psychedelic is DMT, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the DMT is present between about 1 mg and 60 mg, about 0.1 mg/kg to about 0.3 mg/kg, or about 0.1 mg/kg followed by 0.3 mg/kg.

In some embodiments, the serotonin receptor modulator is an extended-release form of risperidone (e.g., RISPERDAL CONSTA) and the psychedelic is DMT, wherein the extended-release form of risperidone (e.g., RISPERDAL CONSTA) is present in about 12.5 mg, or about 25 mg, or about 37.5 mg, or about 50 mg and the DMT is present between about 1 mg and 60 mg or 0.1 mg/kg to about 0.3 mg/kg or 0.1 mg/kg followed by 0.3 mg/kg.

In some embodiments, the serotonin receptor modulator is eplivanserin and the psychedelic is 5-MeO-DMT, wherein the eplivanserin is present in about 1 mg to about 40 mg, or about 5 mg to about 10 mg, and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is volinanserin and the psychedelic is 5-MeO-DMT, wherein the volinanserin is present in about 1 mg to about 60 mg, or about 5 mg to about 20 mg, and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is ketanserin and the psychedelic is 5-MeO-DMT, wherein the ketanserin is present in about 10 mg to about 80 mg, about 30 mg to about 50 mg, or about 40 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is ritanserin and the psychedelic is 5-MeO-DMT, wherein the ritanserin is present in about 1 mg to about 40 mg, or about 2.5 mg to about 10 mg, and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is pimavanserin and the psychedelic is 5-MeO-DMT, wherein the pimavanserin is present in about 1 mg to about 60 mg, or about 17 mg to about 34 mg, and the 5-MeO-DMT is present between about 1 mg and 30 mg, about 0.1 mg/kg to about 0.3 mg/kg, or about 0.1 mg/kg followed by 0.3 mg/kg

In some embodiments, the serotonin receptor modulator is nelotanserin and the psychedelic is 5-MeO-DMT, wherein the nelotanserin is present in about 1 mg to about 80 mg, or about 40 mg to about 80 mg, and the 5-MeO-DMT is present between about 1 mg and 30 mg or 0.1 mg/kg to about 0.3 mg/kg or 0.1 mg/kg followed by 0.3 mg/kg.

In some embodiments, the serotonin receptor modulator is pruvanserin and the psychedelic is 5-MeO-DMT, wherein the pruvanserin is present in about 1 mg to about 40 mg, or about 3 mg to about 10 mg, and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is flibanserin and the psychedelic is 5-MeO-DMT, wherein the flibanserin is present in about 10 mg to about 200 mg, or about 80 mg to about 120 mg, or about 100 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is olanzapine and the psychedelic is 5-MeO-DMT, wherein the olanzapine is present in about 2.5 mg to about 30 mg, or about 5 mg or about 10 mg, or about 20 mg or about 25 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is an extended-release form of olanzapine (e.g., ZYPREXA RELPREVV) and the psychedelic is 5-MeO-DMT, wherein the extended-release form of olanzapine (e.g., ZYPREXA RELPREVV) is present in about 50 mg to about 450 mg, or about 150 mg or about 210 mg, or about 300 mg or about 405 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is quetiapine and the psychedelic is 5-MeO-DMT, wherein the quetiapine is present in about 25 mg to about 800 mg, or about 50 mg to about 100 mg, or about 150 mg or about 200 mg or about 250 mg or about 300 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is an extended-release form of quetiapine and the psychedelic is 5-MeO-DMT, wherein the extended-release form of quetiapine is present in about 50 mg to about 300 mg, or about 50 mg or about 100 mg or about 200 mg, or about 300 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is risperidone and the psychedelic is 5-MeO-DMT, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is an extended-release form of risperidone (e.g., RISPERDAL CONSTA) and the psychedelic is 5-MeO-DMT, wherein the extended-release form of risperidone (e.g., RISPERDAL CONSTA) is present in about 12.5 mg, or about 25 mg, or about 37.5 mg, or about 50 mg and the 5-MeO-DMT is present between about 1 mg and 30 mg, about 0.1 mg/kg to about 0.3 mg/kg, or about 0.1 mg/kg followed by 0.3 mg/kg.

In some embodiments, the serotonin receptor modulator is eplivanserin and the psychedelic is 2C-B, wherein the eplivanserin is present in about 1 mg to about 40 mg, or about 5 mg to about 10 mg, and the 2C-B is present between about 1 mg and 40 mg.

In some embodiments, the serotonin receptor modulator is volinanserin and the psychedelic is 2C-B, wherein the volinanserin is present in about 1 mg to about 60 mg, or about 5 mg to about 20 mg, and the 2C-B is present between about 1 mg and 40 mg.

In some embodiments, the serotonin receptor modulator is ketanserin and the psychedelic is 2C-B, wherein the ketanserin is present in about 10 mg to about 80 mg, about 30 mg to about 50 mg, or about 40 mg and the 2C-B is present between about 1 mg and 40 mg.

In some embodiments, the serotonin receptor modulator is ritanserin and the psychedelic is 2C-B, wherein the ritanserin is present in about 1 mg to about 40 mg, or about 2.5 mg to about 10 mg, and the 2C-B is present between about 1 mg and 40 mg.

In some embodiments, the serotonin receptor modulator is pimavanserin and the psychedelic is 2C-B, wherein the pimavanserin is present in about 1 mg to about 60 mg, or about 17 mg to about 34 mg, and the 2C-B is present between about 1 mg and 40 mg.

In some embodiments, the serotonin receptor modulator is nelotanserin and the psychedelic is 2C-B, wherein the nelotanserin is present in about 1 mg to about 80 mg, or about 40 mg to about 80 mg, and the 2C-B is present between about 1 mg and 40 mg.

In some embodiments, the serotonin receptor modulator is pruvanserin and the psychedelic is 2C-B, wherein the pruvanserin is present in about 1 mg to about 40 mg, or about 3 mg to about 10 mg, and the 2C-B is present between about 1 mg and 40 mg.

In some embodiments, the serotonin receptor modulator is flibanserin and the psychedelic is 2C-B, wherein the flibanserin is present in about 10 mg to about 200 mg, or about 80 mg to about 120 mg, or about 100 mg and the 2C-B is present between about 1 mg and 40 mg.

In some embodiments, the serotonin receptor modulator is olanzapine and the psychedelic is 2C-B, wherein the olanzapine is present in about 2.5 mg to about 30 mg, or about 5 mg or about 10 mg, or about 20 mg or about 25 mg and the 2C-B is present between about 1 mg and 40 mg.

In some embodiments, the serotonin receptor modulator is an extended-release form of olanzapine (e.g., ZYPREXA RELPREVV) and the psychedelic is 2C-B, wherein the extended-release form of olanzapine (e.g., ZYPREXA RELPREVV) is present in about 50 mg to about 450 mg, or about 150 mg or about 210 mg, or about 300 mg or about 405 mg and the 2C-B is present between about 1 mg and 40 mg.

In some embodiments, the serotonin receptor modulator is quetiapine and the psychedelic is 2C-B, wherein the quetiapine is present in about 25 mg to about 800 mg, or about 50 mg to about 100 mg, or about 150 mg or about 200 mg or about 250 mg or about 300 mg and the 2C-B is present between about 1 mg and 40 mg.

In some embodiments, the serotonin receptor modulator is an extended-release form of quetiapine and the psychedelic is 2C-B, wherein the extended-release form of quetiapine is present in about 50 mg to about 300 mg, or about 50 mg or about 100 mg or about 200 mg, or about 300 mg and the 2C-B is present between about 1 mg and 40 mg.

In some embodiments, the serotonin receptor modulator is risperidone and the psychedelic is 2C-B, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the 2C-B is present between about 1 mg and 40 mg.

In some embodiments, the serotonin receptor modulator is an extended-release form of risperidone (e.g., RISPERDAL CONSTA) and the psychedelic is 2C-B, wherein the extended-release form of risperidone (e.g., RISPERDAL CONSTA) is present in about 12.5 mg, or about 25 mg, or about 37.5 mg, or about 50 mg and the 2C-B is present between about 1 mg and 40 mg.

In some embodiments, the serotonin receptor modulator is eplivanserin and the psychedelic is mescaline, wherein the eplivanserin is present in about 1 mg to about 40 mg, or about 5 mg to about 10 mg, and the mescaline is present between about 50 mg and about 800 mg, or between about 200 mg and 500 mg.

In some embodiments, the serotonin receptor modulator is volinanserin and the psychedelic is mescaline, wherein the volinanserin is present in about 1 mg to about 60 mg, or about 5 mg to about 20 mg, and the mescaline is present between about 50 mg and about 800 mg, or between about 200 mg and 500 mg.

In some embodiments, the serotonin receptor modulator is ketanserin and the psychedelic is mescaline, wherein the ketanserin is present in about 10 mg to about 80 mg, about 30 mg to about 50 mg, or about 40 mg and the mescaline is present between about 50 mg and about 800 mg, or between about 200 mg and 500 mg.

In some embodiments, the serotonin receptor modulator is ritanserin and the psychedelic is mescaline, wherein the ritanserin is present in about 1 mg to about 40 mg, or about 2.5 mg to about 10 mg, and the mescaline is present between about 50 mg and about 800 mg, or between about 200 mg and 500 mg.

In some embodiments, the serotonin receptor modulator is pimavanserin and the psychedelic is mescaline, wherein the pimavanserin is present in about 1 mg to about 60 mg, or about 17 mg to about 34 mg, and the mescaline is present between about 50 mg and about 800 mg, or between about 200 mg and 500 mg.

In some embodiments, the serotonin receptor modulator is nelotanserin and the psychedelic is mescaline, wherein the nelotanserin is present in about 1 mg to about 80 mg, or about 40 mg to about 80 mg, and the mescaline is present between about 50 mg and about 800 mg, or between about 200 mg and 500 mg.

In some embodiments, the serotonin receptor modulator is pruvanserin and the psychedelic is mescaline, wherein the pruvanserin is present in about 1 mg to about 40 mg, or about 3 mg to about 10 mg, and the mescaline is present between about 50 mg and about 800 mg, or between about 200 mg and 500 mg.

In some embodiments, the serotonin receptor modulator is flibanserin and the psychedelic is mescaline, wherein the flibanserin is present in about 10 mg to about 200 mg, or about 80 mg to about 120 mg, or about 100 mg and the mescaline is present between about 50 mg and about 800 mg, or between about 200 mg and 500 mg.

In some embodiments, the serotonin receptor modulator is olanzapine and the psychedelic is mescaline, wherein the olanzapine is present in about 2.5 mg to about 30 mg, or about 5 mg or about 10 mg, or about 20 mg or about 25 mg and the mescaline is present between about 50 mg and about 800 mg, or between about 200 mg and 500 mg.

In some embodiments, the serotonin receptor modulator is an extended-release form of olanzapine (e.g., ZYPREXA RELPREVV) and the psychedelic is mescaline, wherein the extended-release form of olanzapine (e.g., ZYPREXA RELPREVV) is present in about 50 mg to about 450 mg, or about 150 mg or about 210 mg, or about 300 mg or about 405 mg and the mescaline is present between about 50 mg and about 800 mg, or between about 200 mg and 500 mg.

In some embodiments, the serotonin receptor modulator is quetiapine and the psychedelic is mescaline, wherein the quetiapine is present in about 25 mg to about 800 mg, or about 50 mg to about 100 mg, or about 150 mg or about 200 mg or about 250 mg or about 300 mg and the mescaline is present between about 50 mg and about 800 mg, or between about 200 mg and 500 mg.

In some embodiments, the serotonin receptor modulator is an extended-release form of quetiapine and the psychedelic is mescaline, wherein the extended-release form of quetiapine is present in about 50 mg to about 300 mg, or about 50 mg or about 100 mg or about 200 mg, or about 300 mg and the mescaline is present between about 50 mg and about 800 mg, or between about 200 mg and 500 mg.

In some embodiments, the serotonin receptor modulator is risperidone and the psychedelic is mescaline, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the mescaline is present between about 50 mg and about 800 mg, or between about 200 mg and 500 mg.

In some embodiments, the serotonin receptor modulator is an extended-release form of risperidone (e.g., RISPERDAL CONSTA) and the psychedelic is mescaline, wherein the extended-release form of risperidone (e.g., RISPERDAL CONSTA) is present in about 12.5 mg, or about 25 mg, or about 37.5 mg, or about 50 mg and the mescaline is present between about 50 mg and about 800 mg, or between about 200 mg and 500 mg.

In some embodiments, the serotonin receptor modulator is eplivanserin and the psychedelic is 12-methoxyibogamine (ibogaine), wherein the eplivanserin is present in about 1 mg to about 40 mg, or about 5 mg to about 10 mg, and the ibogaine is present between about 50 mg and about 1,000 mg, or between about 500 mg and 800 mg.

In some embodiments, the serotonin receptor modulator is volinanserin and the psychedelic is ibogaine, wherein the volinanserin is present in about 1 mg to about 60 mg, or about 5 mg to about 20 mg, and the ibogaine is present between about 50 mg and about 1,000 mg, or between about 500 mg and 800 mg.

In some embodiments, the serotonin receptor modulator is ketanserin and the psychedelic is ibogaine, wherein the ketanserin is present in about 10 mg to about 80 mg, about 30 mg to about 50 mg, or about 40 mg and the ibogaine is present between about 50 mg and about 1,000 mg, or between about 500 mg and 800 mg.

In some embodiments, the serotonin receptor modulator is ritanserin and the psychedelic is ibogaine, wherein the ritanserin is present in about 1 mg to about 40 mg, or about 2.5 mg to about 10 mg, and the ibogaine is present between about 50 mg and about 1,000 mg, or between about 500 mg and 800 mg.

In some embodiments, the serotonin receptor modulator is pimavanserin and the psychedelic is ibogaine, wherein the pimavanserin is present in about 1 mg to about 60 mg, or about 17 mg to about 34 mg, and the ibogaine is present between about 50 mg and about 1,000 mg, or between about 500 mg and 800 mg.

In some embodiments, the serotonin receptor modulator is nelotanserin and the psychedelic is ibogaine, wherein the nelotanserin is present in about 1 mg to about 80 mg, or about 40 mg to about 80 mg, and the ibogaine is present between about 50 mg and about 1,000 mg, or between about 500 mg and 800 mg.

In some embodiments, the serotonin receptor modulator is pruvanserin and the psychedelic is ibogaine, wherein the pruvanserin is present in about 1 mg to about 40 mg, or about 3 mg to about 10 mg, and the ibogaine is present between about 50 mg and about 1,000 mg, or between about 500 mg and 800 mg.

In some embodiments, the serotonin receptor modulator is flibanserin and the psychedelic is ibogaine, wherein the flibanserin is present in about 10 mg to about 200 mg, or about 80 mg to about 120 mg, or about 100 mg and the ibogaine is present between about 50 mg and about 1,000 mg, or between about 500 mg and 800 mg.

In some embodiments, the serotonin receptor modulator is olanzapine and the psychedelic is ibogaine, wherein the olanzapine is present in about 2.5 mg to about 30 mg, or about 5 mg or about 10 mg, or about 20 mg or about 25 mg and the ibogaine is present between about 50 mg and about 1,000 mg, or between about 500 mg and 800 mg.

In some embodiments, the serotonin receptor modulator is an extended-release form of olanzapine (e.g., ZYPREXA RELPREVV) and the psychedelic is ibogaine, wherein the extended-release form of olanzapine (e.g., ZYPREXA RELPREVV) is present in about 50 mg to about 450 mg, or about 150 mg or about 210 mg, or about 300 mg or about 405 mg and the ibogaine is present between about 50 mg and about 1,000 mg, or between about 500 mg and 800 mg.

In some embodiments, the serotonin receptor modulator is quetiapine and the psychedelic is ibogaine, wherein the quetiapine is present in about 25 mg to about 800 mg, or about 50 mg to about 100 mg, or about 150 mg or about 200 mg or about 250 mg or about 300 mg and the ibogaine is present between about 50 mg and about 1,000 mg, or between about 500 mg and 800 mg.

In some embodiments, the serotonin receptor modulator is an extended-release form of quetiapine and the psychedelic is ibogaine, wherein the extended-release form of quetiapine is present in about 50 mg to about 300 mg, or about 50 mg or about 100 mg or about 200 mg, or about 300 mg and the ibogaine is present between about 50 mg and about 1,000 mg, or between about 500 mg and 800 mg.

In some embodiments, the serotonin receptor modulator is risperidone and the psychedelic is ibogaine, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the ibogaine is present between about 50 mg and about 1,000 mg, or between about 500 mg and 800 mg.

In some embodiments, the serotonin receptor modulator is an extended-release form of risperidone (e.g., RISPERDAL CONSTA) and the psychedelic is ibogaine, wherein the extended-release form of risperidone (e.g., RISPERDAL CONSTA) is present in about 12.5 mg, or about 25 mg, or about 37.5 mg, or about 50 mg and the ibogaine is present between about 50 mg and about 1,000 mg, or between about 500 mg and 800 mg.

In some embodiments, the serotonin receptor modulator is eplivanserin and the psychedelic is MDMA, wherein the eplivanserin is present in about 1 mg to about 40 mg, or about 5 mg to about 10 mg, and the MDMA is present between about 50 mg and about 200 mg, or between about 80 mg and 120 mg.

In some embodiments, the serotonin receptor modulator is volinanserin and the psychedelic is MDMA, wherein the volinanserin is present in about 1 mg to about 60 mg, or about 5 mg to about 20 mg, and the MDMA is present between about 50 mg and about 200 mg, or between about 80 mg and 120 mg.

In some embodiments, the serotonin receptor modulator is ketanserin and the psychedelic is MDMA, wherein the ketanserin is present in about 10 mg to about 80 mg, about 30 mg to about 50 mg, or about 40 mg and the MDMA is present between about 50 mg and about 200 mg, or between about 80 mg and 120 mg.

In some embodiments, the serotonin receptor modulator is ritanserin and the psychedelic is MDMA, wherein the ritanserin is present in about 1 mg to about 40 mg, or about 2.5 mg to about 10 mg, and the MDMA is present between about 50 mg and about 200 mg, or between about 80 mg and 120 mg.

In some embodiments, the serotonin receptor modulator is pimavanserin and the psychedelic is MDMA, wherein the pimavanserin is present in about 1 mg to about 60 mg, or about 17 mg to about 34 mg, and the MDMA is present between about 50 mg and about 200 mg, or between about 80 mg and 120 mg.

In some embodiments, the serotonin receptor modulator is nelotanserin and the psychedelic is MDMA, wherein the nelotanserin is present in about 1 mg to about 80 mg, or about 40 mg to about 80 mg, and the MDMA is present between about 50 mg and about 200 mg, or between about 80 mg and 120 mg.

In some embodiments, the serotonin receptor modulator is pruvanserin and the psychedelic is MDMA, wherein the pruvanserin is present in about 1 mg to about 40 mg, or about 3 mg to about 10 mg, and the MDMA is present between about 50 mg and about 200 mg, or between about 80 mg and 120 mg.

In some embodiments, the serotonin receptor modulator is flibanserin and the psychedelic is MDMA, wherein the flibanserin is present in about 10 mg to about 200 mg, or about 80 mg to about 120 mg, or about 100 mg and the MDMA is present between about 50 mg and about 200 mg, or between about 80 mg and 120 mg.

In some embodiments, the serotonin receptor modulator is olanzapine and the psychedelic is MDMA, wherein the olanzapine is present in about 2.5 mg to about 30 mg, or about 5 mg or about 10 mg, or about 20 mg or about 25 mg and the MDMA is present between about 50 mg and about 200 mg, or between about 80 mg and 120 mg.

In some embodiments, the serotonin receptor modulator is an extended-release form of olanzapine (e.g., ZYPREXA RELPREVV) and the psychedelic is MDMA, wherein the extended-release form of olanzapine (e.g., ZYPREXA RELPREVV) is present in about 50 mg to about 450 mg, or about 150 mg or about 210 mg, or about 300 mg or about 405 mg and the MDMA is present between about 50 mg and about 200 mg, or between about 80 mg and 120 mg.

In some embodiments, the serotonin receptor modulator is quetiapine and the psychedelic is MDMA, wherein the quetiapine is present in about 25 mg to about 800 mg, or about 50 mg to about 100 mg, or about 150 mg or about 200 mg or about 250 mg or about 300 mg and the MDMA is present between about 50 mg and about 200 mg, or between about 80 mg and 120 mg.

In some embodiments, the serotonin receptor modulator is an extended-release form of quetiapine and the psychedelic is MDMA, wherein the extended-release form of quetiapine is present in about 50 mg to about 300 mg, or about 50 mg or about 100 mg or about 200 mg, or about 300 mg and the MDMA is present between about 50 mg and about 200 mg, or between about 80 mg and 120 mg.

In some embodiments, the serotonin receptor modulator is risperidone and the psychedelic is MDMA, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the MDMA is present between about 50 mg and about 200 mg, or between about 80 mg and 120 mg.

In some embodiments, the serotonin receptor modulator is an extended-release form of risperidone (e.g., RISPERDAL CONSTA) and the psychedelic is MDMA, wherein the extended-release form of risperidone (e.g., RISPERDAL CONSTA) is present in about 12.5 mg, or about 25 mg, or about 37.5 mg, or about 50 mg and the MDMA is present between about 50 mg and about 200 mg, or between about 80 mg and 120 mg.

In some embodiments, the serotonin receptor modulator is eplivanserin and the psychedelic is DOM, wherein the eplivanserin is present in about 1 mg to about 40 mg, or about 5 mg to about 10 mg, and the DOM is present between about 0.5 mg and about 15 mg, or about 5 mg.

In some embodiments, the serotonin receptor modulator is volinanserin and the psychedelic is DOM, wherein the volinanserin is present in about 1 mg to about 60 mg, or about 5 mg to about 20 mg, and the DOM is present between about 0.5 mg and about 15 mg, or about 5 mg.

In some embodiments, the serotonin receptor modulator is ketanserin and the psychedelic is DOM, wherein the ketanserin is present in about 10 mg to about 80 mg, about 30 mg to about 50 mg, or about 40 mg and the DOM is present between about 0.5 mg and about 15 mg, or about 5 mg.

In some embodiments, the serotonin receptor modulator is ritanserin and the psychedelic is DOM, wherein the ritanserin is present in about 1 mg to about 40 mg, or about 2.5 mg to about 10 mg, and the DOM is present between about 0.5 mg and about 15 mg, or about 5 mg.

In some embodiments, the serotonin receptor modulator is pimavanserin and the psychedelic is DOM, wherein the pimavanserin is present in about 1 mg to about 60 mg, or about 17 mg to about 34 mg, and the DOM is present between about 0.5 mg and about 15 mg, or about 5 mg.

In some embodiments, the serotonin receptor modulator is nelotanserin and the psychedelic is DOM, wherein the nelotanserin is present in about 1 mg to about 80 mg, or about 40 mg to about 80 mg, and the DOM is present between about 0.5 mg and about 15 mg, or about 5 mg.

In some embodiments, the serotonin receptor modulator is pruvanserin and the psychedelic is DOM, wherein the pruvanserin is present in about 1 mg to about 40 mg, or about 3 mg to about 10 mg, and the DOM is present between about 0.5 mg and about 15 mg, or about 5 mg.

In some embodiments, the serotonin receptor modulator is flibanserin and the psychedelic is DOM, wherein the flibanserin is present in about 10 mg to about 200 mg, or about 80 mg to about 120 mg, or about 100 mg and the DOM is present between about 0.5 mg and about 15 mg, or about 5 mg.

In some embodiments, the serotonin receptor modulator is olanzapine and the psychedelic is DOM, wherein the olanzapine is present in about 2.5 mg to about 30 mg, or about 5 mg or about 10 mg, or about 20 mg or about 25 mg and the DOM is present between about 0.5 mg and about 15 mg, or about 5 mg.

In some embodiments, the serotonin receptor modulator is an extended-release form of olanzapine (e.g., ZYPREXA RELPREVV) and the psychedelic is DOM, wherein the extended-release form of olanzapine (e.g., ZYPREXA RELPREVV) is present in about 50 mg to about 450 mg, or about 150 mg or about 210 mg, or about 300 mg or about 405 mg and the DOM is present between about 0.5 mg and about 15 mg, or about 5 mg.

In some embodiments, the serotonin receptor modulator is quetiapine and the psychedelic is DOM, wherein the quetiapine is present in about 25 mg to about 800 mg, or about 50 mg to about 100 mg, or about 150 mg or about 200 mg or about 250 mg or about 300 mg and the DOM is present between about 0.5 mg and about 15 mg, or about 5 mg.

In some embodiments, the serotonin receptor modulator is an extended-release form of quetiapine and the psychedelic is DOM, wherein the extended-release form of quetiapine is present in about 50 mg to about 300 mg, or about 50 mg or about 100 mg or about 200 mg, or about 300 mg and the DOM is present between about 0.5 mg and about 15 mg, or about 5 mg.

In some embodiments, the serotonin receptor modulator is risperidone and the psychedelic is DOM, wherein the risperidone is present in about 0.5 mg to about 20 mg or about 0.5 mg, or about 1 mg, or about 2 mg, or about 3 mg or about 4 mg or about 5 mg or about 7.5 mg or about 10 mg or about 16 mg and the DOM is present between about 0.5 mg and about 15 mg, or about 5 mg.

In some embodiments, the serotonin receptor modulator is an extended-release form of risperidone (e.g., RISPERDAL CONSTA) and the psychedelic is DOM, wherein the extended-release form of risperidone (e.g., RISPERDAL CONSTA) is present in about 12.5 mg, or about 25 mg, or about 37.5 mg, or about 50 mg and the DOM is present between about 0.5 mg and about 15 mg, or about 5 mg.

In some embodiments, the serotonin receptor modulator for use with the psychedelic (R)-MDMA is eplivanserin and, wherein the eplivanserin is administered in about 1 mg to about 40 mg, or about 5 mg to about 10 mg, and the (R)-MDMA is administered between about 50 mg and about 200 mg, or between about 80 mg and 120 mg.

In some embodiments, the serotonin receptor modulator is volinanserin and the psychedelic is (R)-MDMA, wherein the volinanserin is administered in about 1 mg to about 60 mg, or about 5 mg to about 20 mg, and the (R)-MDMA is administered between about 50 mg and about 200 mg, or between about 80 mg and 120 mg.

In some embodiments, the serotonin receptor modulator for use with the psychedelic (R)-MDMA is ketanserin, wherein the ketanserin is administered in about 10 mg to about 80 mg, about 30 mg to about 50 mg, or about 40 mg and the (R)-MDMA is administered between about 50 mg and about 200 mg, or between about 80 mg and 120 mg.

In some embodiments, the serotonin receptor modulator for use with the psychedelic (R)-MDMA is ritanserin, wherein the ritanserin is administered in about 1 mg to about 40 mg, or about 2.5 mg to about 10 mg, and the (R)-MDMA is administered between about 50 mg and about 200 mg, or between about 80 mg and 120 mg.

In some embodiments, the serotonin receptor modulator is pimavanserin and the psychedelic is (R)-MDMA, wherein the pimavanserin is administered in about 1 mg to about 60 mg, or about 17 mg to about 34 mg, and the (R)-MDMA is administered between about 50 mg and about 200 mg, or between about 80 mg and 120 mg.

In some embodiments, the serotonin receptor modulator for use with the psychedelic (R)-MDMA is nelotanserin, wherein the nelotanserin is administered in about 1 mg to about 80 mg, or about 40 mg to about 80 mg, and the (R)-MDMA is administered between about 50 mg and about 200 mg, or between about 80 mg and 120 mg.

In some embodiments, the serotonin receptor modulator for use with the psychedelic (R)-MDMA is pruvanserin, wherein the pruvanserin is administered in about 1 mg to about 40 mg, or about 3 mg to about 10 mg, and the (R)-MDMA is administered between about 50 mg and about 200 mg, or between about 80 mg and 120 mg.

In some embodiments, the serotonin receptor modulator for use with the psychedelic (R)-MDMA is flibanserin, wherein the flibanserin is administered in about 10 mg to about 200 mg, or about 80 mg to about 120 mg, or about 100 mg and the (R)-MDMA is administered between about 50 mg and about 200 mg, or between about 80 mg and 120 mg.

In some embodiments, the serotonin receptor modulator is olanzapine and the psychedelic is (R)-MDMA, wherein the olanzapine is administered in about 2.5 mg to about 30 mg, or about 5 mg or about 10 mg, or about 20 mg or about 25 mg, and the (R)-MDMA is administered between about 50 mg and about 200 mg, or between about 80 mg and 120 mg.

In some embodiments, the serotonin receptor modulator is an extended-release of olanzapine (e.g., ZYPREXA RELPREVV) and the psychedelic is (R)-MDMA, wherein the extended-release form of olanzapine (e.g., ZYPREXA RELPREVV) is administered in about 50 mg to about 450 mg, or about 150 mg or about 210 mg, or about 300 mg or about 405 mg, and the (R)-MDMA is administered between about 50 mg and about 200 mg, or between about 80 mg and 120 mg.

In some embodiments, the serotonin receptor modulator is quetiapine and the psychedelic is (R)-MDMA, wherein the quetiapine is administered in about 25 mg to about 800 mg, or about 50 mg to about 100 mg, or about 150 mg or about 200 mg or about 250 mg or about 300 mg, and the (R)-MDMA is administered between about 50 mg and about 200 mg, or between about 80 mg and 120 mg.

In some embodiments, the serotonin receptor modulator is an extended-release of quetiapine and the psychedelic is (R)-MDMA, wherein the extended-release of quetiapine is administered in about 50 mg to about 300 mg, or about 50 mg or about 100 mg or about 200 mg, or about 300 mg, and the (R)-MDMA is administered between about 50 mg and about 200 mg, or between about 80 mg and 120 mg.

In some embodiments, the serotonin receptor modulator is risperidone and the psychedelic is (R)-MDMA, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the (R)-MDMA is administered between about 50 mg and about 200 mg, or between about 80 mg and 120 mg.

In some embodiments, the serotonin receptor modulator is an extended-release of risperidone (e.g., RISPERDAL CONSTA) and the psychedelic is (R)-MDMA, wherein the extended-release form of risperidone (e.g., RISPERDAL CONSTA) is administered in about 12.5 mg, or about 25 mg, or about 37.5 mg, or about 50 mg, and the (R)-MDMA is administered between about 50 mg and about 200 mg, or between about 80 mg and 120 mg.

In some embodiments, the serotonin receptor modulator for use with the psychedelic (S)-MDMA is eplivanserin and, wherein the eplivanserin is administered in about 1 mg to about 40 mg, or about 5 mg to about 10 mg, and the (S)-MDMA is administered between about 50 mg and about 200 mg, or between about 80 mg and 120 mg.

In some embodiments, the serotonin receptor modulator is volinanserin and the psychedelic is (S)-MDMA, wherein the volinanserin is administered in about 1 mg to about 60 mg, or about 5 mg to about 20 mg, and the (S)-MDMA is administered between about 50 mg and about 200 mg, or between about 80 mg and 120 mg.

In some embodiments, the serotonin receptor modulator for use with the psychedelic (S)-MDMA is ketanserin, wherein the ketanserin is administered in about 10 mg to about 80 mg, about 30 mg to about 50 mg, or about 40 mg and the (S)-MDMA is administered between about 50 mg and about 200 mg, or between about 80 mg and 120 mg.

In some embodiments, the serotonin receptor modulator for use with the psychedelic (S)-MDMA is ritanserin, wherein the ritanserin is administered in about 1 mg to about 40 mg, or about 2.5 mg to about 10 mg, and the (S)-MDMA is administered between about 50 mg and about 200 mg, or between about 80 mg and 120 mg.

In some embodiments, the serotonin receptor modulator is pimavanserin and the psychedelic is (S)-MDMA, wherein the pimavanserin is administered in about 1 mg to about 60 mg, or about 17 mg to about 34 mg, and the (S)-MDMA is administered between about 50 mg and about 200 mg, or between about 80 mg and 120 mg.

In some embodiments, the serotonin receptor modulator for use with the psychedelic (S)-MDMA is nelotanserin, wherein the nelotanserin is administered in about 1 mg to about 80 mg, or about 40 mg to about 80 mg, and the (S)-MDMA is administered between about 50 mg and about 200 mg, or between about 80 mg and 120 mg.

In some embodiments, the serotonin receptor modulator for use with the psychedelic (S)-MDMA is pruvanserin, wherein the pruvanserin is administered in about 1 mg to about 40 mg, or about 3 mg to about 10 mg, and the (S)-MDMA is administered between about 50 mg and about 200 mg, or between about 80 mg and 120 mg.

In some embodiments, the serotonin receptor modulator for use with the psychedelic (S)-MDMA is flibanserin, wherein the flibanserin is administered in about 10 mg to about 200 mg, or about 80 mg to about 120 mg, or about 100 mg and the (S)-MDMA is administered between about 50 mg and about 200 mg, or between about 80 mg and 120 mg.

In some embodiments, the serotonin receptor modulator is olanzapine and the psychedelic is (S)-MDMA, wherein the olanzapine is administered in about 2.5 mg to about 30 mg, or about 5 mg or about 10 mg, or about 20 mg or about 25 mg, and the (S)-MDMA is administered between about 50 mg and about 200 mg, or between about 80 mg and 120 mg.

In some embodiments, the serotonin receptor modulator is an extended-release form of olanzapine (e.g., ZYPREXA RELPREVV) and the psychedelic is (S)-MDMA, wherein the extended-release form of olanzapine (e.g., ZYPREXA RELPREVV) is administered in about 50 mg to about 450 mg, or about 150 mg or about 210 mg, or about 300 mg or about 405 mg, and the (S)-MDMA is administered between about 50 mg and about 200 mg, or between about 80 mg and 120 mg.

In some embodiments, the serotonin receptor modulator is quetiapine and the psychedelic is (S)-MDMA, wherein the quetiapine is administered in about 25 mg to about 800 mg, or about 50 mg to about 100 mg, or about 150 mg or about 200 mg or about 250 mg or about 300 mg, and the (S)-MDMA is administered between about 50 mg and about 200 mg, or between about 80 mg and 120 mg.

In some embodiments, the serotonin receptor modulator is an extended-release form of quetiapine and the psychedelic is (S)-MDMA, wherein the extended-release form of quetiapine is administered in about 50 mg to about 300 mg, or about 50 mg or about 100 mg or about 200 mg, or about 300 mg, and the (S)-MDMA is administered between about 50 mg and about 200 mg, or between about 80 mg and 120 mg.

In some embodiments, the serotonin receptor modulator is risperidone and the psychedelic is (S)-MDMA, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the (S)-MDMA is administered between about 50 mg and about 200 mg, or between about 80 mg and 120 mg.

In some embodiments, the serotonin receptor modulator is an extended-release form of risperidone (e.g., RISPERDAL CONSTA) and the psychedelic is (S)-MDMA, wherein the extended-release form of risperidone (e.g., RISPERDAL CONSTA) is administered in about 12.5 mg, or about 25 mg, or about 37.5 mg, or about 50 mg, and the (S)-MDMA is administered between about 50 mg and about 200 mg, or between about 80 mg and 120 mg.

In some embodiments, the serotonin receptor modulator for use with MBDB is eplivanserin, wherein the eplivanserin is administered in about 1 mg to about 40 mg, or about 5 mg to about 10 mg and MBDB is administered in about 10 mg to about 500 mg or about 150 mg to about 250 mg or about 180 mg or about 210 mg or about 250 mg.

In some embodiments, the serotonin receptor modulator for use with MBDB is volinanserin, wherein the volinanserin is administered in about 1 mg to about 60 mg, or about 5 mg to about 20 mg and MBDB is administered in about 10 mg to about 500 mg or about 150 mg to about 250 mg or about 180 mg or about 210 mg or about 250 mg.

In some embodiments, the serotonin receptor modulator for use with MBDB is ketanserin, wherein the ketanserin is administered in about 10 mg to about 80 mg, about 30 mg to about 50 mg, or about 40 mg and MBDB is administered in about 10 mg to about 500 mg or about 150 mg to about 250 mg or about 180 mg or about 210 mg or about 250 mg.

In some embodiments, the serotonin receptor modulator for use with MBDB is ritanserin, wherein the ritanserin is administered in about 1 mg to about 40 mg, or about 2.5 mg to about 10 mg and MBDB is administered in about 10 mg to about 500 mg or about 150 mg to about 250 mg or about 180 mg or about 210 mg or about 250 mg.

In some embodiments, the serotonin receptor modulator for use with MBDB is pimavanserin, wherein the pimavanserin is administered in about 1 mg to about 60 mg, or about 17 mg to about 34 mg and MBDB is administered in about 10 mg to about 500 mg or about 150 mg to about 250 mg or about 180 mg or about 210 mg or about 250 mg.

In some embodiments, the serotonin receptor modulator for use with MBDB is nelotanserin, wherein the nelotanserin is administered in about 1 mg to about 80 mg, or about 40 mg to about 80 mg and MBDB is administered in about 10 mg to about 500 mg or about 150 mg to about 250 mg or about 180 mg or about 210 mg or about 250 mg.

In some embodiments, the serotonin receptor modulator for use with MBDB is pruvanserin, wherein the pruvanserin is administered in about 1 mg to about 40 mg, or about 3 mg to about 10 mg and MBDB is administered in about 10 mg to about 500 mg or about 150 mg to about 250 mg or about 180 mg or about 210 mg or about 250 mg.

In some embodiments, the serotonin receptor modulator for use with MBDB is flibanserin, wherein the flibanserin is administered in about 10 mg to about 200 mg, or about 80 mg to about 120 mg, or about 100 mg and MBDB is administered in about 10 mg to about 500 mg or about 150 mg to about 250 mg or about 180 mg or about 210 mg or about 250 mg.

In some embodiments, the serotonin receptor modulator is olanzapine and the psychedelic is MBDB, wherein the olanzapine is administered in about 2.5 mg to about 30 mg, or about 5 mg or about 10 mg, or about 20 mg or about 25 mg, and the MBDB is administered in about 10 mg to about 500 mg or about 150 mg to about 250 mg or about 180 mg or about 210 mg or about 250 mg.

In some embodiments, the serotonin receptor modulator is an extended-release form of olanzapine (e.g., ZYPREXA RELPREVV) and the psychedelic is MBDB, wherein the extended-release form of olanzapine (e.g., ZYPREXA RELPREVV) is administered in about 50 mg to about 450 mg, or about 150 mg or about 210 mg, or about 300 mg or about 405 mg, and the MBDB is administered in about 10 mg to about 500 mg or about 150 mg to about 250 mg or about 180 mg or about 210 mg or about 250 mg.

In some embodiments, the serotonin receptor modulator is quetiapine and the psychedelic is MBDB, wherein the quetiapine is administered in about 25 mg to about 800 mg, or about 50 mg to about 100 mg, or about 150 mg or about 200 mg or about 250 mg or about 300 mg, and the MBDB is administered in about 10 mg to about 500 mg or about 150 mg to about 250 mg or about 180 mg or about 210 mg or about 250 mg.

In some embodiments, the serotonin receptor modulator is an extended-release form of quetiapine and the psychedelic is MBDB, wherein the extended-release form of quetiapine is administered in about 50 mg to about 300 mg, or about 50 mg or about 100 mg or about 200 mg, or about 300 mg, and the MBDB is administered in about 10 mg to about 500 mg or about 150 mg to about 250 mg or about 180 mg or about 210 mg or about 250 mg.

In some embodiments, the serotonin receptor modulator is risperidone and the psychedelic is MBDB, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the MBDB is administered in about 10 mg to about 500 mg or about 150 mg to about 250 mg or about 180 mg or about 210 mg or about 250 mg.

In some embodiments, the serotonin receptor modulator is an extended-release form of risperidone (e.g., RISPERDAL CONSTA) and the psychedelic is MBDB, wherein the extended-release form of risperidone (e.g., RISPERDAL CONSTA) is administered in about 12.5 mg, or about 25 mg, or about 37.5 mg, or about 50 mg, and the MBDB is administered in about 10 mg to about 500 mg or about 150 mg to about 250 mg or about 180 mg or about 210 mg or about 250 mg.

In some embodiments, the serotonin receptor modulator for use with methylone disclosed herein is eplivanserin, wherein the eplivanserin is administered in about 1 mg to about 40 mg, or about 5 mg to about 10 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is volinanserin, wherein the volinanserin is administered in about 1 mg to about 60 mg, or about 5 mg to about 20 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is ketanserin, wherein the ketanserin is administered in about 10 mg to about 80 mg, about 30 mg to about 50 mg, or about 40 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is ritanserin, wherein the ritanserin is administered in about 1 mg to about 40 mg, or about 2.5 mg to about 10 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is pimavanserin, wherein the pimavanserin is administered in about 1 mg to about 60 mg, or about 17 mg to about 34 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is nelotanserin, wherein the nelotanserin is administered in about 1 mg to about 80 mg, or about 40 mg to about 80 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is pruvanserin, wherein the pruvanserin is administered in about 1 mg to about 40 mg, or about 3 mg to about 10 mg and the methylone is administered in about 10 mg to about 500 mg or about 100 mg to about 250 mg or about 120 mg or about 150 mg or about 180 mg or about 250 mg.

In some embodiments, the serotonin receptor modulator for use with the methylone is flibanserin, wherein the flibanserin is administered in about 10 mg to about 200 mg, or about 80 mg to about 120 mg, or about 100 mg and the methylone is administered in about 10 mg to about 500 mg or about 100 mg to about 250 mg or about 120 mg or about 150 mg or about 180 mg or about 250 mg.

In some embodiments, the serotonin receptor modulator is olanzapine and the psychedelic is methylone, wherein the olanzapine is administered in about 2.5 mg to about 30 mg, or about 5 mg or about 10 mg, or about 20 mg or about 25 mg, and the methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of olanzapine (e.g., ZYPREXA RELPREVV) and the psychedelic is methylone, wherein the extended-release form of olanzapine (e.g., ZYPREXA RELPREVV) is administered in about 50 mg to about 450 mg, or about 150 mg or about 210 mg, or about 300 mg or about 405 mg, and the methylone is administered in about 10 mg to about 500 mg or about 100 mg to about 250 mg or about 120 mg or about 150 mg or about 180 mg or about 250 mg.

In some embodiments, the serotonin receptor modulator is quetiapine and the psychedelic is methylone, wherein the quetiapine is administered in about 25 mg to about 800 mg, or about 50 mg to about 100 mg, or about 150 mg or about 200 mg or about 250 mg or about 300 mg, and the methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of quetiapine and the psychedelic is methylone, wherein the extended-release form of quetiapine is administered in about 50 mg to about 300 mg, or about 50 mg or about 100 mg or about 200 mg, or about 300 mg, and the methylone is administered in about 10 mg to about 500 mg or about 100 mg to about 250 mg or about 120 mg or about 150 mg or about 180 mg or about 250 mg.

In some embodiments, the serotonin receptor modulator is risperidone and the psychedelic is methylone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of risperidone (e.g., RISPERDAL CONSTA) and the psychedelic is methylone, wherein the extended-release form of risperidone (e.g., RISPERDAL CONSTA) is administered in about 12.5 mg, or about 25 mg, or about 37.5 mg, or about 50 mg, and the methylone is administered in about 10 mg to about 500 mg or about 100 mg to about 250 mg or about 120 mg or about 150 mg or about 180 mg or about 250 mg.

In some embodiments, the serotonin receptor modulator for use with (R)-methylone disclosed herein is eplivanserin, wherein the eplivanserin is administered in about 1 mg to about 40 mg, or about 5 mg to about 10 mg and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is volinanserin, wherein the volinanserin is administered in about 1 mg to about 60 mg, or about 5 mg to about 20 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is ketanserin, wherein the ketanserin is administered in about 10 mg to about 80 mg, about 30 mg to about 50 mg, or about 40 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is ritanserin, wherein the ritanserin is administered in about 1 mg to about 40 mg, or about 2.5 mg to about 10 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is pimavanserin, wherein the pimavanserin is administered in about 1 mg to about 60 mg, or about 17 mg to about 34 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is nelotanserin, wherein the nelotanserin is administered in about 1 mg to about 80 mg, or about 40 mg to about 80 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is pruvanserin, wherein the pruvanserin is administered in about 1 mg to about 40 mg, or about 3 mg to about 10 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with the (R)-methylone is flibanserin, wherein the flibanserin is administered in about 10 mg to about 200 mg, or about 80 mg to about 120 mg, or about 100 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is olanzapine and the psychedelic is (R)-methylone, wherein the olanzapine is administered in about 2.5 mg to about 30 mg, or about 5 mg or about 10 mg, or about 20 mg or about 25 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of olanzapine (e.g., ZYPREXA RELPREVV) and the psychedelic is (R)-methylone, wherein the extended-release form of olanzapine (e.g., ZYPREXA RELPREVV) is administered in about 50 mg to about 450 mg, or about 150 mg or about 210 mg, or about 300 mg or about 405 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is quetiapine and the psychedelic is (R)-methylone, wherein the quetiapine is administered in about 25 mg to about 800 mg, or about 50 mg to about 100 mg, or about 150 mg or about 200 mg or about 250 mg or about 300 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of quetiapine and the psychedelic is (R)-methylone, wherein the extended-release form of quetiapine is administered in about 50 mg to about 300 mg, or about 50 mg or about 100 mg or about 200 mg, or about 300 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is risperidone and the psychedelic is (R)-methylone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of risperidone (e.g., RISPERDAL CONSTA) and the psychedelic is (R)-methylone, wherein the extended-release form of risperidone (e.g., RISPERDAL CONSTA) is administered in about 12.5 mg, or about 25 mg, or about 37.5 mg, or about 50 mg, and the (R)-methylone is administered in about 10 mg to about 500 mg or about 100 mg to about 250 mg or about 120 mg or about 150 mg or about 180 mg or about 250 mg.

In some embodiments, the serotonin receptor modulator for use with (S)-methylone disclosed herein is eplivanserin, wherein the eplivanserin is administered in about 1 mg to about 40 mg, or about 5 mg to about 10 mg and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is volinanserin, wherein the volinanserin is administered in about 1 mg to about 60 mg, or about 5 mg to about 20 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is ketanserin, wherein the ketanserin is administered in about 10 mg to about 80 mg, about 30 mg to about 50 mg, or about 40 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is ritanserin, wherein the ritanserin is administered in about 1 mg to about 40 mg, or about 2.5 mg to about 10 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is pimavanserin, wherein the pimavanserin is administered in about 1 mg to about 60 mg, or about 17 mg to about 34 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is nelotanserin, wherein the nelotanserin is administered in about 1 mg to about 80 mg, or about 40 mg to about 80 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is pruvanserin, wherein the pruvanserin is administered in about 1 mg to about 40 mg, or about 3 mg to about 10 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with the (S)-methylone is flibanserin, wherein the flibanserin is administered in about 10 mg to about 200 mg, or about 80 mg to about 120 mg, or about 100 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is olanzapine and the psychedelic is (S)-methylone, wherein the olanzapine is administered in about 2.5 mg to about 30 mg, or about 5 mg or about 10 mg, or about 20 mg or about 25 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of olanzapine (e.g., ZYPREXA RELPREVV) and the psychedelic is (S)-methylone, wherein the extended-release form of olanzapine (e.g., ZYPREXA RELPREVV) is administered in about 50 mg to about 450 mg, or about 150 mg or about 210 mg, or about 300 mg or about 405 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is quetiapine and the psychedelic is (S)-methylone, wherein the quetiapine is administered in about 25 mg to about 800 mg, or about 50 mg to about 100 mg, or about 150 mg or about 200 mg or about 250 mg or about 300 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of quetiapine and the psychedelic is (S)-methylone, wherein the extended-release form of quetiapine is administered in about 50 mg to about 300 mg, or about 50 mg or about 100 mg or about 200 mg, or about 300 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is risperidone and the psychedelic is (S)-methylone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator is an extended-release form of risperidone (e.g., RISPERDAL CONSTA) and the psychedelic is (S)-methylone, wherein the extended-release form of risperidone (e.g., RISPERDAL CONSTA) is administered in about 12.5 mg, or about 25 mg, or about 37.5 mg, or about 50 mg, and the (S)-methylone is administered in about 10 mg to about 500 mg or about 100 mg to about 250 mg or about 120 mg or about 150 mg or about 180 mg or about 250 mg.

In some embodiments, the serotonin receptor modulator for use with N-ethyl-3,4-methylenedioxyamphetamine (MDEA) is eplivanserin, wherein the eplivanserin is administered in about 1 mg to about 40 mg, or about 5 mg to about 10 mg and MDEA is administered in about 10 mg to about 300 mg or about 100 mg to about 180 mg or about 120 mg or about 150 mg or about 160 mg.

In some embodiments, the serotonin receptor modulator for use with the N-ethyl-3,4-methylenedioxyamphetamine (MDEA) is volinanserin, wherein the volinanserin is administered in about 1 mg to about 60 mg, or about 5 mg to about 20 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is ketanserin, wherein the ketanserin is administered in about 10 mg to about 80 mg, about 30 mg to about 50 mg, or about 40 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is ritanserin, wherein the ritanserin is administered in about 1 mg to about 40 mg, or about 2.5 mg to about 10 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is pimavanserin, wherein the pimavanserin is administered in about 1 mg to about 60 mg, or about 17 mg to about 34 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is nelotanserin, wherein the nelotanserin is administered in about 1 mg to about 80 mg, or about 40 mg to about 80 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is pruvanserin, wherein the pruvanserin is administered in about 1 mg to about 40 mg, or about 3 mg to about 10 mg and MDEA is administered in about 10 mg to about 300 mg or about 100 mg to about 180 mg or about 120 mg or about 150 mg or about 160 mg.

In some embodiments, the serotonin receptor modulator for use with N-ethyl-3,4-methylenedioxyamphetamine (MDEA) is flibanserin, wherein the flibanserin is administered in about 10 mg to about 200 mg, or about 80 mg to about 120 mg, or about 100 mg and MDEA is administered in about 10 mg to about 300 mg or about 100 mg to about 180 mg or about 120 mg or about 150 mg or about 160 mg.

In some embodiments, the serotonin receptor modulator is olanzapine and the psychedelic is N-ethyl-3,4-methylenedioxyamphetamine (MDEA), wherein the olanzapine is administered in about 2.5 mg to about 30 mg, or about 5 mg or about 10 mg, or about 20 mg or about 25 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator is an extended-release form of olanzapine (e.g., ZYPREXA RELPREVV) and the psychedelic is N-ethyl-3,4-methylenedioxyamphetamine (MDEA), wherein the extended-release form of olanzapine (e.g., ZYPREXA RELPREVV) is administered in about 50 mg to about 450 mg, or about 150 mg or about 210 mg, or about 300 mg or about 405 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator is quetiapine and the psychedelic is N-ethyl-3,4-methylenedioxyamphetamine (MDEA), wherein the quetiapine is administered in about 25 mg to about 800 mg, or about 50 mg to about 100 mg, or about 150 mg or about 200 mg or about 250 mg or about 300 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator is an extended-release form of quetiapine and the psychedelic is N-ethyl-3,4-methylenedioxyamphetamine (MDEA), wherein the extended-release form of quetiapine is administered in about 50 mg to about 300 mg, or about 50 mg or about 100 mg or about 200 mg, or about 300 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator is risperidone and the psychedelic is N-ethyl-3,4-methylenedioxyamphetamine (MDEA), wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator is an extended-release form of risperidone (e.g., RISPERDAL CONSTA) and the psychedelic is N-ethyl-3,4-methylenedioxyamphetamine (MDEA), wherein the extended-release form of risperidone (e.g., RISPERDAL CONSTA) is administered in about 12.5 mg, or about 25 mg, or about 37.5 mg, or about 50 mg, and the N-ethyl-3,4-methylenedioxyamphetamine (MDEA) is administered in about 10 mg to about 300 mg or about 100 mg to about 180 mg or about 120 mg or about 150 mg or about 160 mg.

In some embodiments, the serotonin receptor modulator for use with (S)—N-ethyl-3,4-methylenedioxyamphetamine (S-MDEA) is eplivanserin, wherein the eplivanserin is administered in about 1 mg to about 40 mg, or about 5 mg to about 10 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is volinanserin, wherein the volinanserin is administered in about 1 mg to about 60 mg, or about 5 mg to about 20 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is ketanserin, wherein the ketanserin is administered in about 10 mg to about 80 mg, about 30 mg to about 50 mg, or about 40 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is ritanserin, wherein the ritanserin is administered in about 1 mg to about 40 mg, or about 2.5 mg to about 10 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is pimavanserin, wherein the pimavanserin is administered in about 1 mg to about 60 mg, or about 17 mg to about 34 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is nelotanserin, wherein the nelotanserin is administered in about 1 mg to about 80 mg, or about 40 mg to about 80 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is pruvanserin, wherein the pruvanserin is administered in about 1 mg to about 40 mg, or about 3 mg to about 10 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator for use with the (S)—N-ethyl-3,4-methylenedioxyamphetamine (S-MDEA) is flibanserin, wherein the flibanserin is administered in about 10 mg to about 200 mg, or about 80 mg to about 120 mg, or about 100 mg and the (S)—N-ethyl-3,4-methylenedioxyamphetamine (S-MDEA) disclosed herein is administered in about 10 mg to about 300 mg or about 100 mg to about 180 mg or about 120 mg or about 150 mg or about 160 mg.

In some embodiments, the serotonin receptor modulator is olanzapine and the psychedelic is (S)—N-ethyl-3,4-methylenedioxyamphetamine (S-MDEA), wherein the olanzapine is administered in about 2.5 mg to about 30 mg, or about 5 mg or about 10 mg, or about 20 mg or about 25 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator is an extended-release form of olanzapine (e.g., ZYPREXA RELPREVV) and the psychedelic is (S)—N-ethyl-3,4-methylenedioxyamphetamine (S-MDEA), wherein the extended-release form of olanzapine (e.g., ZYPREXA RELPREVV) is administered in about 50 mg to about 450 mg, or about 150 mg or about 210 mg, or about 300 mg or about 405 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator is quetiapine and the psychedelic is (S)—N-ethyl-3,4-methylenedioxyamphetamine (S-MDEA), wherein the quetiapine is administered in about 25 mg to about 800 mg, or about 50 mg to about 100 mg, or about 150 mg or about 200 mg or about 250 mg or about 300 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator is an extended-release form of quetiapine and the psychedelic is (S)—N-ethyl-3,4-methylenedioxyamphetamine (S-MDEA), wherein the extended-release form of quetiapine is administered in about 50 mg to about 300 mg, or about 50 mg or about 100 mg or about 200 mg, or about 300 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator is risperidone and the psychedelic is (S)—N-ethyl-3,4-methylenedioxyamphetamine (S-MDEA), wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator is an extended-release form of risperidone (e.g., RISPERDAL CONSTA) and the psychedelic is (S)—N-ethyl-3,4-methylenedioxyamphetamine (S-MDEA), wherein the extended-release form of risperidone (e.g., RISPERDAL CONSTA) is administered in about 12.5 mg, or about 25 mg, or about 37.5 mg, or about 50 mg, and the (S)—N-ethyl-3,4-methylenedioxyamphetamine (S-MDEA) is administered in about 10 mg to about 300 mg or about 100 mg to about 180 mg or about 120 mg or about 150 mg or about 160 mg.

In some embodiments, the serotonin receptor modulator for use with N-ethyl-2-(5-fluoro-1H-indol-3-yl)-N-methylethan-1-amine is eplivanserin, wherein the eplivanserin is administered in about 1 mg to about 40 mg, or about 5 mg to about 10 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-1H-indol-3-yl)-N-methylethan-1-amine is volinanserin, wherein the volinanserin is administered in about 1 mg to about 60 mg, or about 5 mg to about 20 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-1H-indol-3-yl)-N-methylethan-1-amine is ketanserin, wherein the ketanserin is administered in about 10 mg to about 80 mg, about 30 mg to about 50 mg, or about 40 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-TH-indol-3-yl)-N-methylethan-1-amine is ritanserin, wherein the ritanserin is administered in about 1 mg to about 40 mg, or about 2.5 mg to about 10 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-1H-indol-3-yl)-N-methylethan-1-amine is pimavanserin, wherein the pimavanserin is administered in about 1 mg to about 60 mg, or about 17 mg to about 34 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-TH-indol-3-yl)-N-methylethan-1-amine is nelotanserin, wherein the nelotanserin is administered in about 1 mg to about 80 mg, or about 40 mg to about 80 mg, and the N-ethyl-2-(5-fluoro-TH-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-TH-indol-3-yl)-N-methylethan-1-amine is pruvanserin, wherein the pruvanserin is administered in about 1 mg to about 40 mg, or about 3 mg to about 10 mg, and the N-ethyl-2-(5-fluoro-TH-indol-3-yl)-N-methylethan-1-amine is administered from about 100 ug to 100 mg, or from 1 mg to 20 mg, or from 15 to 30 mg.

In some embodiments, the serotonin receptor modulator for use with the N-ethyl-2-(5-fluoro-TH-indol-3-yl)-N-methylethan-1-amine is flibanserin, wherein the flibanserin is administered in about 10 mg to about 200 mg, or about 80 mg to about 120 mg, or about 100 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is olanzapine and the psychedelic is N-ethyl-2-(5-fluoro-1H-indol-3-yl)-N-methylethan-1-amine, wherein the olanzapine is administered in about 2.5 mg to about 30 mg, or about 5 mg or about 10 mg, or about 20 mg or about 25 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is an extended-release form of olanzapine (e.g., ZYPREXA RELPREVV) and the psychedelic is N-ethyl-2-(5-fluoro-1H-indol-3-yl)-N-methylethan-1-amine, wherein the extended-release form of olanzapine (e.g., ZYPREXA RELPREVV) is administered in about 50 mg to about 450 mg, or about 150 mg or about 210 mg, or about 300 mg or about 405 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is quetiapine and the psychedelic is N-ethyl-2-(5-fluoro-1H-indol-3-yl)-N-methylethan-1-amine, wherein the quetiapine is administered in about 25 mg to about 800 mg, or about 50 mg to about 100 mg, or about 150 mg or about 200 mg or about 250 mg or about 300 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is an extended-release form of quetiapine and the psychedelic is N-ethyl-2-(5-fluoro-1H-indol-3-yl)-N-methylethan-1-amine, wherein the extended-release form of quetiapine is administered in about 50 mg to about 300 mg, or about 50 mg or about 100 mg or about 200 mg, or about 300 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is risperidone and the psychedelic is N-ethyl-2-(5-fluoro-1H-indol-3-yl)-N-methylethan-1-amine, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator is an extended-release form of risperidone (e.g., RISPERDAL CONSTA) and the psychedelic is N-ethyl-2-(5-fluoro-1H-indol-3-yl)-N-methylethan-1-amine, wherein the extended-release form of risperidone (e.g., RISPERDAL CONSTA) is administered in about 12.5 mg, or about 25 mg, or about 37.5 mg, or about 50 mg, and the N-ethyl-2-(5-fluoro-1H-indol-3-yl)-N-methylethan-1-amine is administered from about 100 ug to 100 mg, or from 1 mg to 20 mg, or from 15 to 30 mg.

In some embodiments, the serotonin receptor modulator for use with 4-OH-DIPT hemi-glutarate is eplivanserin, wherein the eplivanserin is administered in about 1 mg to about 40 mg, or about 5 mg to about 10 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is volinanserin, wherein the volinanserin is administered in about 1 mg to about 60 mg, or about 5 mg to about 20 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate hydrochloride disclosed herein is ketanserin, wherein the ketanserin is administered in about 10 mg to about 80 mg, about 30 mg to about 50 mg, or about 40 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is ritanserin, wherein the ritanserin is administered in about 1 mg to about 40 mg, or about 2.5 mg to about 10 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is pimavanserin, wherein the pimavanserin is administered in about 1 mg to about 60 mg, or about 17 mg to about 34 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is nelotanserin, wherein the nelotanserin is administered in about 1 mg to about 80 mg, or about 40 mg to about 80 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is pruvanserin, wherein the pruvanserin is administered in about 1 mg to about 40 mg, or about 3 mg to about 10 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with 4-OH-DIPT hemi-glutarate is flibanserin, wherein the flibanserin is administered in about 10 mg to about 200 mg, or about 80 mg to about 120 mg, or about 100 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is olanzapine and the psychedelic is 4-OH-DIPT hemi-glutarate, wherein the olanzapine is administered in about 2.5 mg to about 30 mg, or about 5 mg or about 10 mg, or about 20 mg or about 25 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is an extended-release form of olanzapine (e.g., ZYPREXA RELPREVV) and the psychedelic is 4-OH-DIPT hemi-glutarate, wherein the extended-release form of olanzapine (e.g., ZYPREXA RELPREVV) is administered in about 50 mg to about 450 mg, or about 150 mg or about 210 mg, or about 300 mg or about 405 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is quetiapine and the psychedelic is 4-OH-DIPT hemi-glutarate, wherein the quetiapine is administered in about 25 mg to about 800 mg, or about 50 mg to about 100 mg, or about 150 mg or about 200 mg or about 250 mg or about 300 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is an extended-release form of quetiapine and the psychedelic is 4-OH-DIPT hemi-glutarate, wherein the extended-release form of quetiapine is administered in about 50 mg to about 300 mg, or about 50 mg or about 100 mg or about 200 mg, or about 300 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is risperidone and the psychedelic is 4-OH-DIPT hemi-glutarate, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator is an extended-release form of risperidone (e.g., RISPERDAL CONSTA) and the psychedelic is 4-OH-DIPT hemi-glutarate, wherein the extended-release form of risperidone (e.g., RISPERDAL CONSTA) is administered in about 12.5 mg, or about 25 mg, or about 37.5 mg, or about 50 mg, and the 4-OH-DIPT hemi-glutarate is administered in about 1 mg to about 50 mg, or about 3 mg to about 10 mg, 15 mg, 20 mg, 25 mg, or 30 mg.

In some embodiments, the serotonin receptor modulator for use with the psychedelic 5,6-dimethoxy-2-aminoindane is eplivanserin and, wherein the eplivanserin is administered in about 1 mg to about 40 mg, or about 5 mg to about 10 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is volinanserin and the psychedelic is 5,6-dimethoxy-2-aminoindane, wherein the volinanserin is administered in about 1 mg to about 60 mg, or about 5 mg to about 20 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5,6-dimethoxy-2-aminoindane is ketanserin, wherein the ketanserin is administered in about 10 mg to about 80 mg, about 30 mg to about 50 mg, or about 40 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5,6-dimethoxy-2-aminoindane is ritanserin, wherein the ritanserin is administered in about 1 mg to about 40 mg, or about 2.5 mg to about 10 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is pimavanserin and the psychedelic is 5,6-dimethoxy-2-aminoindane, wherein the pimavanserin is administered in about 1 mg to about 60 mg, or about 17 mg to about 34 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5,6-dimethoxy-2-aminoindane is nelotanserin, wherein the nelotanserin is administered in about 1 mg to about 80 mg, or about 40 mg to about 80 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5,6-dimethoxy-2-aminoindane is pruvanserin, wherein the pruvanserin is administered in about 1 mg to about 40 mg, or about 3 mg to about 10 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5,6-dimethoxy-2-aminoindane is flibanserin, wherein the flibanserin is administered in about 10 mg to about 200 mg, or about 80 mg to about 120 mg, or about 100 mg and 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is olanzapine and the psychedelic is 5,6-dimethoxy-2-aminoindane, wherein the olanzapine is administered in about 2.5 mg to about 30 mg, or about 5 mg or about 10 mg, or about 20 mg or about 25 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of olanzapine (e.g., ZYPREXA RELPREVV) and the psychedelic is 5,6-dimethoxy-2-aminoindane, wherein the extended-release form of olanzapine (e.g., ZYPREXA RELPREVV) is administered in about 50 mg to about 450 mg, or about 150 mg or about 210 mg, or about 300 mg or about 405 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is quetiapine and the psychedelic is 5,6-dimethoxy-2-aminoindane, wherein the quetiapine is administered in about 25 mg to about 800 mg, or about 50 mg to about 100 mg, or about 150 mg or about 200 mg or about 250 mg or about 300 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of quetiapine and the psychedelic is 5,6-dimethoxy-2-aminoindane, wherein the extended-release form of quetiapine is administered in about 50 mg to about 300 mg, or about 50 mg or about 100 mg or about 200 mg, or about 300 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is risperidone and the psychedelic is 5,6-dimethoxy-2-aminoindane, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of risperidone (e.g., RISPERDAL CONSTA) and the psychedelic is 5,6-dimethoxy-2-aminoindane, wherein the extended-release form of risperidone (e.g., RISPERDAL CONSTA) is administered in about 12.5 mg, or about 25 mg, or about 37.5 mg, or about 50 mg, and the 5,6-dimethoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is eplivanserin and, wherein the eplivanserin is administered in about 1 mg to about 40 mg, or about 5 mg to about 10 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is volinanserin and the psychedelic is 5-methoxy-2-aminoindane, wherein the volinanserin is administered in about 1 mg to about 60 mg, or about 5 mg to about 20 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is ketanserin, wherein the ketanserin is administered in about 10 mg to about 80 mg, about 30 mg to about 50 mg, or about 40 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is ritanserin, wherein the ritanserin is administered in about 1 mg to about 40 mg, or about 2.5 mg to about 10 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is pimavanserin and the psychedelic is 5-methoxy-2-aminoindane, wherein the pimavanserin is administered in about 1 mg to about 60 mg, or about 17 mg to about 34 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is nelotanserin, wherein the nelotanserin is administered in about 1 mg to about 80 mg, or about 40 mg to about 80 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is pruvanserin, wherein the pruvanserin is administered in about 1 mg to about 40 mg, or about 3 mg to about 10 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with the psychedelic 5-methoxy-2-aminoindane is flibanserin, wherein the flibanserin is administered in about 10 mg to about 200 mg, or about 80 mg to about 120 mg, or about 100 mg and 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is olanzapine and the psychedelic is 5-methoxy-2-aminoindane, wherein the olanzapine is administered in about 2.5 mg to about 30 mg, or about 5 mg or about 10 mg, or about 20 mg or about 25 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of olanzapine (e.g., ZYPREXA RELPREVV) and the psychedelic is 5-methoxy-2-aminoindane, wherein the extended-release form of olanzapine (e.g., ZYPREXA RELPREVV) is administered in about 50 mg to about 450 mg, or about 150 mg or about 210 mg, or about 300 mg or about 405 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is quetiapine and the psychedelic is 5-methoxy-2-aminoindane, wherein the quetiapine is administered in about 25 mg to about 800 mg, or about 50 mg to about 100 mg, or about 150 mg or about 200 mg or about 250 mg or about 300 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of quetiapine and the psychedelic is 5-methoxy-2-aminoindane, wherein the extended-release form of quetiapine is administered in about 50 mg to about 300 mg, or about 50 mg or about 100 mg or about 200 mg, or about 300 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is risperidone and the psychedelic is 5-methoxy-2-aminoindane, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator is an extended-release form of risperidone (e.g., RISPERDAL CONSTA) and the psychedelic is 5-methoxy-2-aminoindane, wherein the extended-release form of risperidone (e.g., RISPERDAL CONSTA) is administered in about 12.5 mg, or about 25 mg, or about 37.5 mg, or about 50 mg, and the 5-methoxy-2-aminoindane is administered in about 20 mg to about 300 mg or about 60 mg to about 180 mg or about 160 mg to about 300 mg or about 1.6 mg per kg to about 4.8 mg per kg or about 99.2 mg to about 297.6 mg.

In some embodiments, the serotonin receptor modulator for use with 2-Br-LSD is eplivanserin, wherein the eplivanserin is administered in about 1 mg to about 40 mg, or about 5 mg to about 10 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is volinanserin, wherein the volinanserin is administered in about 1 mg to about 60 mg, or about 5 mg to about 20 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is ketanserin, wherein the ketanserin is administered in about 10 mg to about 80 mg, about 30 mg to about 50 mg, or about 40 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is ritanserin, wherein the ritanserin is administered in about 1 mg to about 40 mg, or about 2.5 mg to about 10 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is pimavanserin, wherein the pimavanserin is administered in about 1 mg to about 60 mg, or about 17 mg to about 34 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is nelotanserin, wherein the nelotanserin is administered in about 1 mg to about 80 mg, or about 40 mg to about 80 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is pruvanserin, wherein the pruvanserin is administered in about 1 mg to about 40 mg, or about 3 mg to about 10 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with 2-Br-LSD is flibanserin, wherein the flibanserin is administered in about 10 mg to about 200 mg, or about 80 mg to about 120 mg, or about 100 mg and 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is olanzapine and the psychedelic is 2-Br-LSD, wherein the olanzapine is administered in about 2.5 mg to about 30 mg, or about 5 mg or about 10 mg, or about 20 mg or about 25 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is an extended-release form of olanzapine (e.g., ZYPREXA RELPREVV) and the psychedelic is 2-Br-LSD, wherein the extended-release form of olanzapine (e.g., ZYPREXA RELPREVV) is administered in about 50 mg to about 450 mg, or about 150 mg or about 210 mg, or about 300 mg or about 405 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is quetiapine and the psychedelic is 2-Br-LSD, wherein the quetiapine is administered in about 25 mg to about 800 mg, or about 50 mg to about 100 mg, or about 150 mg or about 200 mg or about 250 mg or about 300 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is an extended-release form of quetiapine and the psychedelic is 2-Br-LSD, wherein the extended-release form of quetiapine is administered in about 50 mg to about 300 mg, or about 50 mg or about 100 mg or about 200 mg, or about 300 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is risperidone and the psychedelic is 2-Br-LSD, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator is an extended-release form of risperidone (e.g., RISPERDAL CONSTA) and the psychedelic is 2-Br-LSD, wherein the extended-release form of risperidone (e.g., RISPERDAL CONSTA) is administered in about 12.5 mg, or about 25 mg, or about 37.5 mg, or about 50 mg, and the 2-Br-LSD is administered in about 10 micrograms to 1.0 mg or about 1.8 mg or about 30 micrograms per kg or about 0.1 mg to about 50 mg or about 1 mg to about 20 mg or 20 mg to about 30 mg.

In some embodiments, the serotonin receptor modulator for use with the psychedelic MDAI is eplivanserin and, wherein the eplivanserin is administered in about 1 mg to about 40 mg, or about 5 mg to about 10 mg and MDAI is administered in about 40 mg to about 300 mg or about 60 mg to about 180 mg or about 60 mg or about 80 mg or about 100 mg or about 180 mg.

In some embodiments, the serotonin receptor modulator is volinanserin and the psychedelic is MDAI, wherein the volinanserin is administered in about 1 mg to about 60 mg, or about 5 mg to about 20 mg and MDAI is administered in about 40 mg to about 300 mg or about 60 mg to about 180 mg or about 60 mg or about 80 mg or about 100 mg or about 180 mg.

In some embodiments, the serotonin receptor modulator for use with the psychedelic MDAI is ketanserin, wherein the ketanserin is administered in about 10 mg to about 80 mg, about 30 mg to about 50 mg, or about 40 mg and MDAI is administered in about 40 mg to about 300 mg or about 60 mg to about 180 mg or about 60 mg or about 80 mg or about 100 mg or about 180 mg.

In some embodiments, the serotonin receptor modulator for use with the psychedelic MDAI is ritanserin, wherein the ritanserin is administered in about 1 mg to about 40 mg, or about 2.5 mg to about 10 mg and MDAI is administered in about 40 mg to about 300 mg or about 60 mg to about 180 mg or about 60 mg or about 80 mg or about 100 mg or about 180 mg.

In some embodiments, the serotonin receptor modulator is pimavanserin and the psychedelic is MDAI, wherein the pimavanserin is administered in about 1 mg to about 60 mg, or about 17 mg to about 34 mg and MDAI is administered in about 40 mg to about 300 mg or about 60 mg to about 180 mg or about 60 mg or about 80 mg or about 100 mg or about 180 mg.

In some embodiments, the serotonin receptor modulator for use with the psychedelic MDAI is nelotanserin, wherein the nelotanserin is administered in about 1 mg to about 80 mg, or about 40 mg to about 80 mg and MDAI is administered in about 40 mg to about 300 mg or about 60 mg to about 180 mg or about 60 mg or about 80 mg or about 100 mg or about 180 mg.

In some embodiments, the serotonin receptor modulator for use with the psychedelic MDAI is pruvanserin, wherein the pruvanserin is administered in about 1 mg to about 40 mg, or about 3 mg to about 10 mg and MDAI is administered in about 40 mg to about 300 mg or about 60 mg to about 180 mg or about 60 mg or about 80 mg or about 100 mg or about 180 mg.

In some embodiments, the serotonin receptor modulator for use with the psychedelic MDAI is flibanserin, wherein the flibanserin is administered in about 10 mg to about 200 mg, or about 80 mg to about 120 mg, or about 100 mg and MDAI is administered in about 40 mg to about 300 mg or about 60 mg to about 180 mg or about 60 mg or about 80 mg or about 100 mg or about 180 mg.

In some embodiments, the serotonin receptor modulator is olanzapine and the psychedelic is MDAI, wherein the olanzapine is administered in about 2.5 mg to about 30 mg, or about 5 mg or about 10 mg, or about 20 mg or about 25 mg, and the MDAI is administered in about 40 mg to about 300 mg or about 60 mg to about 180 mg or about 60 mg or about 80 mg or about 100 mg or about 180 mg.

In some embodiments, the serotonin receptor modulator is an extended-release form of olanzapine (e.g., ZYPREXA RELPREVV) and the psychedelic is MDAI, wherein the extended-release form of olanzapine (e.g., ZYPREXA RELPREVV) is administered in about 50 mg to about 450 mg, or about 150 mg or about 210 mg, or about 300 mg or about 405 mg, and the MDAI is administered in about 40 mg to about 300 mg or about 60 mg to about 180 mg or about 60 mg or about 80 mg or about 100 mg or about 180 mg.

In some embodiments, the serotonin receptor modulator is quetiapine and the psychedelic is MDAI, wherein the quetiapine is administered in about 25 mg to about 800 mg, or about 50 mg to about 100 mg, or about 150 mg or about 200 mg or about 250 mg or about 300 mg, and the MDAI is administered in about 40 mg to about 300 mg or about 60 mg to about 180 mg or about 60 mg or about 80 mg or about 100 mg or about 180 mg.

In some embodiments, the serotonin receptor modulator is an extended-release form of quetiapine and the psychedelic is MDAI, wherein the extended-release form of quetiapine is administered in about 50 mg to about 300 mg, or about 50 mg or about 100 mg or about 200 mg, or about 300 mg, and the MDAI is administered in about 40 mg to about 300 mg or about 60 mg to about 180 mg or about 60 mg or about 80 mg or about 100 mg or about 180 mg.

In some embodiments, the serotonin receptor modulator is risperidone and the psychedelic is MDAI, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the MDAI is administered in about 40 mg to about 300 mg or about 60 mg to about 180 mg or about 60 mg or about 80 mg or about 100 mg or about 180 mg.

In some embodiments, the serotonin receptor modulator is an extended-release form of risperidone (e.g., RISPERDAL CONSTA) and the psychedelic is MDAI, wherein the extended-release form of risperidone (e.g., RISPERDAL CONSTA) is administered in about 12.5 mg, or about 25 mg, or about 37.5 mg, or about 50 mg, and the MDAI is administered in about 40 mg to about 300 mg or about 60 mg to about 180 mg or about 60 mg or about 80 mg or about 100 mg or about 180 mg.

Described herein, in some embodiments, are compositions comprising a psychedelic and a serotonin receptor modulator, wherein the psychedelic and the serotonin receptor modulator are provided in a single form or single unit dosage form. In some embodiments, the single form is in the form of a pill, ampoule, vial, or tablet.

### Methods of Use

In some embodiments, a serotonin receptor modulator provided herein and a psychedelic are administered in a same formulation. In other embodiments, a serotonin receptor modulator provided herein and a psychedelic are administered in different formulations. In some embodiments, the serotonin receptor modulator is administered in a first pharmaceutical formulation, and the psychedelic is administered in a second pharmaceutical formulation. In some embodiments, the first pharmaceutical formulation and the second pharmaceutical formulation are suitable for the same administration route (e.g., oral, intravenous, intranasal, etc.). In some embodiments, the first pharmaceutical formulation and the second pharmaceutical formulation are suitable for different administration routes (e.g., oral, intravenous, intranasal, etc., respectively).

In some embodiments, a serotonin receptor modulator provided herein and a psychedelic are administered in a fixed dose combination (e.g., a solid dosage form). In some embodiments, the fixed dosage combination comprises a) an immediate release (IR) first dosage composition comprising a psychedelic and a first excipient; and b) a delay release (DR) second dosage composition comprising a serotonin receptor modulator and a second excipient. In some embodiments, the psychedelic is released first and then the serotonin receptor modulator is released either at the same time or at a later timepoint when the patient experiences a set duration of the psychedelic trip and the administration of the serotonin receptor modulator ends the trip sooner as compared to no administration of serotonin receptor modulator after the administration of the psychedelic. In some embodiments, the psychedelic is released first and then the serotonin receptor modulator is released either at the same time or at a later timepoint when the patient experiences a set duration of the psychedelic trip and the administration reduces intensity of the trip effects in the patient.

In some embodiments, the fixed dose combination can be administered in any appropriate route, including a parenteral (e.g., subcutaneous, intramuscular, intravenous, or inhalation) route. In some embodiments, the compositions are prepared by any method known in the art of pharmacy, for example by mixing the active ingredient with the carrier(s) or excipient(s) under sterile conditions.

In another aspect, provided herein are method of reducing psychedelic trip effects in a patient induced by a psychedelic comprising administering a serotonin receptor modulator to the patient wherein the patient has been administered a psychedelic. In some embodiments, the patient has psychedelic trip after administration of the psychedelic.

In another aspect, provided herein are methods of treating a disease or disorder in a subject in need thereof, the method comprising administering to the subject a composition comprising: a psychedelic; a serotonin receptor modulator; and an excipient, wherein the serotonin receptor modulator is released at most about 3 hours post to the release of the psychedelic. In some embodiments, the disease or disorder is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

Described herein are methods of treating depression or a disease or disorder related to depression in a subject in need thereof, the method comprising administering to the subject a composition comprising: a psychedelic; a serotonin receptor modulator; and an excipient, wherein the serotonin receptor modulator is released at most about 3 hours post to the release of the psychedelic. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, methods of treating depression or a disease or disorder related to depression comprise treating the symptoms associated with the depression or the disease or disorder related to depression.

Methods as described herein, in some embodiments, comprise administering composition comprising a psychedelic and a serotonin receptor modulator, wherein the serotonin receptor modulator attenuates the activation of the serotonin receptor. In some embodiments, the serotonin receptor modulator attenuates the activation of the serotonin receptor that results in psychedelic-induced perceptual alterations but does not attenuate the activation of the serotonin receptor resulting in neural changes. In some embodiments, the serotonin receptor modulator attenuates the activation of the serotonin receptor by the psychedelic. In some embodiments, the serotonin receptor modulator attenuates the activation of the serotonin receptor by 5-95%, 10-90%, 20-80%, 30-70%, 40-60%, 50-95%, 65-85%, or 75-95%. In some embodiments, the serotonin receptor modulator attenuates the activation of the serotonin receptor by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator attenuates the activation of the serotonin receptor by at least about or about 5%. In some embodiments, the serotonin receptor modulator attenuates the activation of the serotonin receptor by at least about or about 10%. In some embodiments, the serotonin receptor modulator attenuates the activation of the serotonin receptor by at least about or about 50%. In some embodiments, the serotonin receptor modulator substantially blocks or blocks the activation of the serotonin receptor. In some embodiments, the serotonin receptor modulator substantially blocks or blocks the activation of the serotonin receptor by the psychedelic.

In some embodiments, methods as described herein comprise administering composition comprising a psychedelic and a serotonin receptor modulator, wherein the serotonin receptor modulator attenuates the activation of the serotonin receptor post to administration of the psychedelic. In some embodiments, the serotonin receptor modulator attenuates the activation of the serotonin receptor by 5-95%, 10-90%, 20-80%, 30-70%, 40-60%, 50-95%, 65-85%, or 75-95% post to administration of the psychedelic. In some embodiments, the serotonin receptor modulator attenuates the activation of the serotonin receptor by at least about or about 5%, 10%, 15%, 20%, 25%, 30%, 35%, 40%, 45%, 50%, 55%, 60%, 65%0, 70%, 75%, 80%, 85%, 90%, 95%, or greater than 95% post to administration of the psychedelic. In some embodiments, the serotonin receptor modulator attenuates the activation of the serotonin receptor by at least about or about 5% post to administration of the psychedelic. In some embodiments, the serotonin receptor modulator attenuates the activation of the serotonin receptor by at least about or about 10% post to administration of the psychedelic. In some embodiments, the serotonin receptor modulator attenuates the activation of the serotonin receptor by at least about or about 50% post to administration of the psychedelic. In some embodiments, the serotonin receptor modulator substantially blocks or blocks the activation of the serotonin receptor post to administration of the psychedelic.

Methods as described herein, in some embodiments, comprise administering composition comprising a psychedelic and a serotonin receptor modulator, wherein the serotonin receptor modulator attenuates the psychedelic-induced perceptual alterations. In some embodiments, psychedelic-induced perceptual alterations comprise changes in consciousness. In some embodiments, the serotonin receptor modulator attenuates the psychedelic-induced perceptual alterations by 5-95%, 10-90%, 20-80%, 30-70%, 40-60%, 50-95%, 65-85%, or 75-95%. In some embodiments, the serotonin receptor modulator attenuates the psychedelic-induced perceptual alterations by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator attenuates the psychedelic-induced perceptual alterations by at least about or about 5%. In some embodiments, the serotonin receptor modulator attenuates the psychedelic-induced perceptual alterations by at least about or about 10%. In some embodiments, the serotonin receptor modulator attenuates the psychedelic-induced perceptual alterations by at least about or about 50%. In some embodiments, the serotonin receptor modulator substantially blocks or blocks the psychedelic-induced perceptual alterations.

Methods as described herein, in some embodiments, comprise administering composition comprising a psychedelic and a serotonin receptor modulator, wherein the serotonin receptor modulator enhances the anti-depressive effects of the psychedelic. In some embodiments, the serotonin receptor modulator improves the anti-depressive effects of the psychedelic by 5-95%, 10-90%, 20-80%, 30-70%, 40-60%, 50-95%, 65-85%, or 75-95%. In some embodiments, the serotonin receptor modulator improves the anti-depressive effects of the psychedelic by at least about or about 5%, 10%, 15%, 20%, 25%, 30%, 35%, 40%, 45%, 50%, 55%, 60%, 65%, 70%, 75%, 80%, 85%, 90%, 95%, or greater than 95%. In some embodiments, the serotonin receptor modulator improves the anti-depressive effects of the psychedelic by at least about or about 5%. In some embodiments, the serotonin receptor modulator improves the anti-depressive effects of the psychedelic by at least about or about 10%. In some embodiments, the serotonin receptor modulator improves the anti-depressive effects of the psychedelic by at least about or about 50%.

In some embodiments, the serotonin receptor modulator attenuates the activation of the serotonin receptor when the serotonin receptor modulator is administered or released from the composition provided herein to post-treat post to the release of the psychedelic for a certain time. In some embodiments, the serotonin receptor modulator attenuates the activation of the serotonin receptor when the serotonin receptor modulator is administered or released from the composition provided herein to post-treat at an early time point (e.g., at most about 1.5 hours). In some embodiments, the serotonin receptor modulator attenuates the activation of the serotonin receptor when the serotonin receptor modulator is administered or released from the composition provided herein to post-treat at most about 2 hours post to the release of the psychedelic. In some embodiments, the serotonin receptor modulator attenuates the activation of the serotonin receptor when the serotonin receptor modulator is administered or released from the composition provided herein to post-treat at most about 1 hour post to the release of the psychedelic. In some embodiments, the serotonin receptor modulator attenuates the activation of the serotonin receptor when the serotonin receptor modulator is administered or released from the composition provided herein to post-treat at most about 1.5 hours post to the release of the psychedelic. In some embodiments, the serotonin receptor modulator attenuates the activation of the serotonin receptor when the serotonin receptor modulator is administered or released from the composition provided herein to post-treat at about 1 hour post to the release of the psychedelic. In some embodiments, the serotonin receptor modulator attenuates the activation of the serotonin receptor when the serotonin receptor modulator is used to post-treat at about or most about 5 minutes, 10 minutes, 20 minutes, 30 minutes, 40 minutes, 50 minutes, 1 hour, 1.25 hours, 1.5 hours, 2 hours, or 3 hours post to the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours post to the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour post to the release of the psychedelic.

In another aspect, also provided herein are methods of treating a disease or disorder in a subject in need thereof, the method comprising administering to the subject a psychedelic and a serotonin receptor modulator, wherein the serotonin receptor is administered post to the administration of the psychedelic, and wherein the serotonin receptor modulator is administered at most about 3 hours post to the administration of the psychedelic. In some embodiments, the disease or disorder is depression or a disease or disorder related to depression.

In another aspect, also provided herein are methods of treating a disease or disorder in a subject in need thereof, the method comprising administering to the subject a psychedelic and a serotonin receptor modulator, wherein the serotonin receptor is administered to the subject post to the administration of the psychedelic to post-treat the subject, and wherein the serotonin receptor modulator is administered at most about 3 hours post to the administration of the psychedelic. In some embodiments, the disease or disorder is depression or a disease or disorder related to depression.

In another aspect, also provided herein are methods of treating a disease or disorder in a subject in need thereof, the methods comprising (i) administering to the subject a serotonin receptor modulator to post-treat the subject, and (ii) administering to the subject a psychedelic, and wherein the serotonin receptor modulator is administered at most about 3 hours post to the administration of the psychedelic. In some embodiments, the disease or disorder is depression or a disease or disorder related to depression.

In yet another aspect, also provided herein are methods of treating a disease or disorder in a subject in need thereof, the methods comprising administering to the subject a psychedelic, wherein the subject has been post-treated by a serotonin receptor modulator, and wherein the serotonin receptor modulator is administered at most about 3 hours post to the administration of the psychedelic. In some embodiments, the disease or disorder is depression or a disease or disorder related to depression.

In yet another aspect, also provided herein are methods of reducing the adverse effects (e.g., hallucination) of a psychedelic in the treatment of a disease or disorder in a subject in need thereof, the methods comprising (i) administering to the subject a serotonin receptor modulator to post-treat the subject, and (ii) administering to the subject a psychedelic, and wherein the serotonin receptor modulator is administered at most about 3 hours post to the administration of the psychedelic. In some embodiments, the disease or disorder is depression or a disease or disorder related to depression.

In yet another aspect, also provided herein are methods of reducing the adverse effects (e.g., hallucination) of a psychedelic in the treatment of a disease or disorder in a subject in need thereof, the methods comprising administering to the subject a psychedelic and a serotonin receptor modulator, wherein the serotonin receptor is administered post to the administration of the psychedelic, and wherein the serotonin receptor modulator is administered at most about 3 hours post to the administration of the psychedelic. In some embodiments, the disease or disorder is depression or a disease or disorder related to depression.

Also provided herein are methods of treating a disease or disorder in a subject in need thereof, the methods comprising administering to the subject a psychedelic, wherein the subject has been post-treated by a serotonin receptor modulator, wherein the serotonin receptor modulator is administered at most about 3 hours post to the administration of the psychedelic, and wherein the serotonin receptor modulator suppresses the adverse effects (e.g., hallucination). In some embodiments, the disease or disorder is depression or a disease or disorder related to depression.

In some embodiments, a benzodiazepine is not administered to the subject. In some embodiments, the subject has not been administered a benzodiazepine. In some embodiments, the subject is not administered or co-administered a benzodiazepine.

In some embodiments, the benzodiazepine is lorazepam, prazepam, flurazepam, quazepam, triazolam, clonazepam, chlordiazepoxide, halazepam, estazolam, temazepam, clorazepate, oxazepam, diazepam, midazolam, or alprazolam. In one embodiment, the benzodiazepine is lorazepam.

In certain embodiments of the methods provided herein, the co-administration of the psychedelic and the serotonin receptor modulator are therapeutically effective in treating a disease or disorder. In some embodiments, the disease or disorder is depression or a disease or disorder related to depression.

In some embodiments of the methods provided herein, the serotonin receptor modulator is administered at most about 3 hours post to the administration of the psychedelic. In some embodiments, the serotonin receptor modulator is administered at most about 2.5 hours post to the administration of the psychedelic. In some embodiments, the serotonin receptor modulator is administered at most about 2 hours post to the administration of the psychedelic. In some embodiments, the serotonin receptor modulator is administered at most about 1.5 hours post to the administration of the psychedelic. In some embodiments, the serotonin receptor modulator is administered at most about 1 hour post to the administration of the psychedelic. In some embodiments, the serotonin receptor modulator is administered at about 1 hour post to the release of the psychedelic. In some embodiments, the serotonin receptor modulator is administered at least about 0.5 hours post to the administration of the psychedelic. In some embodiments, the serotonin receptor modulator is administered at least about 1 hour post to the release of the psychedelic. In some embodiments, the serotonin receptor modulator is administered at most about 5 minutes, 10 minutes, 20 minutes, 30 minutes, 40 minutes, 50 minutes, 1 hour, 1.25 hours, 1.5 hours, 2 hours, or 3 hours post to the administration of the psychedelic. In some embodiments, the serotonin receptor modulator is administered at most about 3 hours, 4 hours, 5 hours, 6 hours, 7 hours, 8 hours, 9 hours, or more than 9 hours post to the administration of the psychedelic. In some embodiments, the serotonin receptor modulator is administered in a window of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 70 minutes to about 3 hours, about 80 minutes to about 3 hours, about 90 minutes to about 3 hours, about 100 minutes to about 3 hours, about 110 minutes to about 3 hours, about 2 hours to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 70 minutes to about 2 hours, about 80 minutes to about 2 hours, about 90 minutes to about 2 hours, about 100 minutes to about 2 hours, about 110 minutes to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour post to the administration of the psychedelic.

In some embodiments, the serotonin receptor modulator is administered at most about 6 hours post to the administration of the psychedelic. In some embodiments, the serotonin receptor modulator is administered at most about 5 hours post to the administration of the psychedelic. In some embodiments, the serotonin receptor modulator is administered at most about 4 hours post to the administration of the psychedelic. In some embodiments, the serotonin receptor modulator is administered at most about 3 hours post to the administration of the psychedelic.

In some embodiments, the serotonin receptor modulator is administered at least about 5 minutes, 10 minutes, 20 minutes, 30 minutes, 40 minutes, 50 minutes, 60 minutes, 70 minutes, 80 minutes, 90 minutes, 100 minutes, 110 minutes, 2 hours, 2.5 hours or 3 hours post to the administration of the psychedelic. In some embodiments, the serotonin receptor modulator is administered at least about 3 hours, 4 hours, 5 hours, 6 hours, 7 hours, 8 hours, 9 hours, or more than 9 hours post to the administration of the psychedelic.

In a preferred embodiment, the serotonin receptor modulator is administered at about 1 hour to about 3 hours post to the administration of the psychedelic.

In some embodiments, the serotonin receptor modulator is eplivanserin and the psychedelic is psilocybin, wherein the eplivanserin is administered to post-treat at least 15 minutes post to the administration of psilocybin. In some embodiments, the serotonin receptor modulator is eplivanserin and the psychedelic is psilocybin, wherein the eplivanserin is administered to post-treat at least 30 minutes post to the psilocybin. In some embodiments, the serotonin receptor modulator is eplivanserin and the psychedelic is psilocybin, wherein the eplivanserin is administered to post-treat at least 60 minutes post to the psilocybin. In some embodiments, the serotonin receptor modulator is eplivanserin and the psychedelic is psilocybin, wherein the eplivanserin is administered to post-treat at least 90 minutes post to the psilocybin. In some embodiments, the serotonin receptor modulator is eplivanserin and the psychedelic is psilocybin, wherein the eplivanserin is administered to post-treat at least 120 minutes post to the psilocybin. In some embodiments, the serotonin receptor modulator is eplivanserin and the psychedelic is psilocybin, wherein the eplivanserin is administered to post-treat at least 150 minutes post to the psilocybin. In some embodiments, the serotonin receptor modulator is eplivanserin and the psychedelic is psilocybin, wherein the eplivanserin is administered to post-treat between about 15 minutes and about 150 minutes post to the psilocybin. In some embodiments, the serotonin receptor modulator is eplivanserin and the psychedelic is psilocybin, wherein the eplivanserin is administered to post-treat at least 180 minutes post to the psilocybin. In some embodiments, the serotonin receptor modulator is eplivanserin and the psychedelic is psilocybin, wherein the eplivanserin is administered to post-treat at least 210 minutes post to the psilocybin. In some embodiments, the serotonin receptor modulator is eplivanserin and the psychedelic is psilocybin, wherein the eplivanserin is administered to post-treat at least 240 minutes post to the psilocybin. In some embodiments, the serotonin receptor modulator is eplivanserin and the psychedelic is psilocybin, wherein the eplivanserin is administered to post-treat at least 270 minutes post to the psilocybin. In some embodiments, the serotonin receptor modulator is eplivanserin and the psychedelic is psilocybin, wherein the eplivanserin is administered to post-treat at least 300 minutes post to the psilocybin. In some embodiments, the serotonin receptor modulator is eplivanserin and the psychedelic is psilocybin, wherein the eplivanserin is administered to post-treat at least 330 minutes post to the psilocybin. In some embodiments, the serotonin receptor modulator is eplivanserin and the psychedelic is psilocybin, wherein the eplivanserin is administered to post-treat at least 360 minutes post to the psilocybin.

In some preferred embodiments, the serotonin receptor modulator is eplivanserin and the psychedelic is psilocybin, wherein eplivanserin is administered to post-treat between about 60 minutes and about 180 minutes post to the administration of psilocybin.

In some embodiments, the serotonin receptor modulator is volinanserin and the psychedelic is psilocybin, wherein the volinanserin is administered to post-treat at least 15 minutes post to the administration of psilocybin. In some embodiments, the serotonin receptor modulator is volinanserin and the psychedelic is psilocybin, wherein the volinanserin is administered to post-treat at least 30 minutes post to the psilocybin. In some embodiments, the serotonin receptor modulator is volinanserin and the psychedelic is psilocybin, wherein the volinanserin is administered to post-treat at least 60 minutes post to the psilocybin. In some embodiments, the serotonin receptor modulator is volinanserin and the psychedelic is psilocybin, wherein the volinanserin is administered to post-treat at least 90 minutes post to the psilocybin. In some embodiments, the serotonin receptor modulator is volinanserin and the psychedelic is psilocybin, wherein the volinanserin is administered to post-treat at least 120 minutes post to the psilocybin. In some embodiments, the serotonin receptor modulator is volinanserin and the psychedelic is psilocybin, wherein the volinanserin is administered to post-treat at least 150 minutes post to the psilocybin. In some embodiments, the serotonin receptor modulator is volinanserin and the psychedelic is psilocybin, wherein the volinanserin is administered to post-treat between about 15 minutes and about 150 minutes post to the psilocybin. In some embodiments, the serotonin receptor modulator is volinanserin and the psychedelic is psilocybin, wherein the volinanserin is administered to post-treat at least 180 minutes post to the psilocybin. In some embodiments, the serotonin receptor modulator is volinanserin and the psychedelic is psilocybin, wherein the volinanserin is administered to post-treat at least 210 minutes post to the psilocybin. In some embodiments, the serotonin receptor modulator is volinanserin and the psychedelic is psilocybin, wherein the volinanserin is administered to post-treat at least 240 minutes post to the psilocybin. In some embodiments, the serotonin receptor modulator is volinanserin and the psychedelic is psilocybin, wherein the volinanserin is administered to post-treat at least 270 minutes post to the psilocybin. In some embodiments, the serotonin receptor modulator is volinanserin and the psychedelic is psilocybin, wherein the volinanserin is administered to post-treat at least 300 minutes post to the psilocybin. In some embodiments, the serotonin receptor modulator is volinanserin and the psychedelic is psilocybin, wherein the volinanserin is administered to post-treat at least 330 minutes post to the psilocybin. In some embodiments, the serotonin receptor modulator is volinanserin and the psychedelic is psilocybin, wherein the volinanserin is administered to post-treat at least 360 minutes post to the psilocybin. In some preferred embodiments, the serotonin receptor modulator is volinanserin and the psychedelic is psilocybin, wherein volinanserin is administered to post-treat between about 60 minutes and about 180 minutes post to the administration of psilocybin.

In some embodiments, the serotonin receptor modulator is ketanserin and the psychedelic is psilocybin, wherein the ketanserin is administered to post-treat at least 15 minutes post to the administration of psilocybin. In some embodiments, the serotonin receptor modulator is ketanserin and the psychedelic is psilocybin, wherein the ketanserin is administered to post-treat at least 30 minutes post to the psilocybin. In some embodiments, the serotonin receptor modulator is ketanserin and the psychedelic is psilocybin, wherein the ketanserin is administered to post-treat at least 60 minutes post to the psilocybin. In some embodiments, the serotonin receptor modulator is ketanserin and the psychedelic is psilocybin, wherein the ketanserin is administered to post-treat at least 90 minutes post to the psilocybin. In some embodiments, the serotonin receptor modulator is ketanserin and the psychedelic is psilocybin, wherein the ketanserin is administered to post-treat at least 120 minutes post to the psilocybin. In some embodiments, the serotonin receptor modulator is ketanserin and the psychedelic is psilocybin, wherein the ketanserin is administered to post-treat at least 150 minutes post to the psilocybin. In some embodiments, the serotonin receptor modulator is ketanserin and the psychedelic is psilocybin, wherein the ketanserin is administered to post-treat between about 15 minutes and about 150 minutes post to the psilocybin. In some embodiments, the serotonin receptor modulator is ketanserin and the psychedelic is psilocybin, wherein the ketanserin is administered to post-treat at least 180 minutes post to the psilocybin. In some embodiments, the serotonin receptor modulator is ketanserin and the psychedelic is psilocybin, wherein the ketanserin is administered to post-treat at least 210 minutes post to the psilocybin. In some embodiments, the serotonin receptor modulator is ketanserin and the psychedelic is psilocybin, wherein the ketanserin is administered to post-treat at least 240 minutes post to the psilocybin. In some embodiments, the serotonin receptor modulator is ketanserin and the psychedelic is psilocybin, wherein the ketanserin is administered to post-treat at least 270 minutes post to the psilocybin. In some embodiments, the serotonin receptor modulator is ketanserin and the psychedelic is psilocybin, wherein the ketanserin is administered to post-treat at least 300 minutes post to the psilocybin. In some embodiments, the serotonin receptor modulator is ketanserin and the psychedelic is psilocybin, wherein the ketanserin is administered to post-treat at least 330 minutes post to the psilocybin. In some embodiments, the serotonin receptor modulator is ketanserin and the psychedelic is psilocybin, wherein the ketanserin is administered to post-treat at least 360 minutes post to the psilocybin. In some preferred embodiments, the serotonin receptor modulator is ketanserin and the psychedelic is psilocybin, wherein ketanserin is administered to post-treat between about 60 minutes and about 180 minutes post to the administration of psilocybin.

In some embodiments, the serotonin receptor modulator is ritanserin and the psychedelic is psilocybin, wherein the ritanserin is administered to post-treat at least 15 minutes post to the administration of psilocybin. In some embodiments, the serotonin receptor modulator is ritanserin and the psychedelic is psilocybin, wherein the ritanserin is administered to post-treat at least 30 minutes post to the psilocybin. In some embodiments, the serotonin receptor modulator is ritanserin and the psychedelic is psilocybin, wherein the ritanserin is administered to post-treat at least 60 minutes post to the psilocybin. In some embodiments, the serotonin receptor modulator is ritanserin and the psychedelic is psilocybin, wherein the ritanserin is administered to post-treat at least 90 minutes post to the psilocybin. In some embodiments, the serotonin receptor modulator is ritanserin and the psychedelic is psilocybin, wherein the ritanserin is administered to post-treat at least 120 minutes post to the psilocybin. In some embodiments, the serotonin receptor modulator is ritanserin and the psychedelic is psilocybin, wherein the ritanserin is administered to post-treat at least 150 minutes post to the psilocybin. In some embodiments, the serotonin receptor modulator is ritanserin and the psychedelic is psilocybin, wherein the ritanserin is administered to post-treat between about 15 minutes and about 150 minutes post to the psilocybin. In some embodiments, the serotonin receptor modulator is ritanserin and the psychedelic is psilocybin, wherein the ritanserin is administered to post-treat at least 180 minutes post to the psilocybin. In some embodiments, the serotonin receptor modulator is ritanserin and the psychedelic is psilocybin, wherein the ritanserin is administered to post-treat at least 210 minutes post to the psilocybin. In some embodiments, the serotonin receptor modulator is ritanserin and the psychedelic is psilocybin, wherein the ritanserin is administered to post-treat at least 240 minutes post to the psilocybin. In some embodiments, the serotonin receptor modulator is ritanserin and the psychedelic is psilocybin, wherein the ritanserin is administered to post-treat at least 270 minutes post to the psilocybin. In some embodiments, the serotonin receptor modulator is ritanserin and the psychedelic is psilocybin, wherein the ritanserin is administered to post-treat at least 300 minutes post to the psilocybin. In some embodiments, the serotonin receptor modulator is ritanserin and the psychedelic is psilocybin, wherein the ritanserin is administered to post-treat at least 330 minutes post to the psilocybin. In some embodiments, the serotonin receptor modulator is ritanserin and the psychedelic is psilocybin, wherein the ritanserin is administered to post-treat at least 360 minutes post to the psilocybin. In some preferred embodiments, the serotonin receptor modulator is ritanserin and the psychedelic is psilocybin, wherein ritanserin is administered to post-treat between about 60 minutes and about 180 minutes post to the administration of psilocybin.

In some embodiments, the serotonin receptor modulator is pimavanserin and the psychedelic is psilocybin, wherein the pimavanserin is administered to post-treat at least 15 minutes post to the administration of psilocybin. In some embodiments, the serotonin receptor modulator is pimavanserin and the psychedelic is psilocybin, wherein the pimavanserin is administered to post-treat at least 30 minutes post to the psilocybin. In some embodiments, the serotonin receptor modulator is pimavanserin and the psychedelic is psilocybin, wherein the pimavanserin is administered to post-treat at least 60 minutes post to the psilocybin. In some embodiments, the serotonin receptor modulator is pimavanserin and the psychedelic is psilocybin, wherein the pimavanserin is administered to post-treat at least 90 minutes post to the psilocybin. In some embodiments, the serotonin receptor modulator is pimavanserin and the psychedelic is psilocybin, wherein the pimavanserin is administered to post-treat at least 120 minutes post to the psilocybin. In some embodiments, the serotonin receptor modulator is pimavanserin and the psychedelic is psilocybin, wherein the pimavanserin is administered to post-treat at least 150 minutes post to the psilocybin. In some embodiments, the serotonin receptor modulator is pimavanserin and the psychedelic is psilocybin, wherein the pimavanserin is administered to post-treat between about 15 minutes and about 150 minutes post to the psilocybin. In some embodiments, the serotonin receptor modulator is pimavanserin and the psychedelic is psilocybin, wherein the pimavanserin is administered to post-treat at least 180 minutes post to the psilocybin. In some embodiments, the serotonin receptor modulator is pimavanserin and the psychedelic is psilocybin, wherein the pimavanserin is administered to post-treat at least 210 minutes post to the psilocybin. In some embodiments, the serotonin receptor modulator is pimavanserin and the psychedelic is psilocybin, wherein the pimavanserin is administered to post-treat at least 240 minutes post to the psilocybin. In some embodiments, the serotonin receptor modulator is pimavanserin and the psychedelic is psilocybin, wherein the pimavanserin is administered to post-treat at least 270 minutes post to the psilocybin. In some embodiments, the serotonin receptor modulator is pimavanserin and the psychedelic is psilocybin, wherein the pimavanserin is administered to post-treat at least 300 minutes post to the psilocybin. In some embodiments, the serotonin receptor modulator is pimavanserin and the psychedelic is psilocybin, wherein the pimavanserin is administered to post-treat at least 330 minutes post to the psilocybin. In some embodiments, the serotonin receptor modulator is pimavanserin and the psychedelic is psilocybin, wherein the pimavanserin is administered to post-treat at least 360 minutes post to the psilocybin. In some preferred embodiments, the serotonin receptor modulator is pimavanserin and the psychedelic is psilocybin, wherein pimavanserin is administered to post-treat between about 60 minutes and about 180 minutes post to the administration of psilocybin.

In some embodiments, the serotonin receptor modulator is nelotanserin and the psychedelic is psilocybin, wherein the nelotanserin is administered to post-treat at least 15 minutes post to the administration of psilocybin. In some embodiments, the serotonin receptor modulator is nelotanserin and the psychedelic is psilocybin, wherein the nelotanserin is administered to post-treat at least 30 minutes post to the psilocybin. In some embodiments, the serotonin receptor modulator is nelotanserin and the psychedelic is psilocybin, wherein the nelotanserin is administered to post-treat at least 60 minutes post to the psilocybin. In some embodiments, the serotonin receptor modulator is nelotanserin and the psychedelic is psilocybin, wherein the nelotanserin is administered to post-treat at least 90 minutes post to the psilocybin. In some embodiments, the serotonin receptor modulator is nelotanserin and the psychedelic is psilocybin, wherein the nelotanserin is administered to post-treat at least 120 minutes post to the psilocybin. In some embodiments, the serotonin receptor modulator is nelotanserin and the psychedelic is psilocybin, wherein the nelotanserin is administered to post-treat at least 150 minutes post to the psilocybin. In some embodiments, the serotonin receptor modulator is nelotanserin and the psychedelic is psilocybin, wherein the nelotanserin is administered to post-treat between about 15 minutes and about 150 minutes post to the psilocybin. In some embodiments, the serotonin receptor modulator is nelotanserin and the psychedelic is psilocybin, wherein the nelotanserin is administered to post-treat at least 180 minutes post to the psilocybin. In some embodiments, the serotonin receptor modulator is nelotanserin and the psychedelic is psilocybin, wherein the nelotanserin is administered to post-treat at least 210 minutes post to the psilocybin. In some embodiments, the serotonin receptor modulator is nelotanserin and the psychedelic is psilocybin, wherein the nelotanserin is administered to post-treat at least 240 minutes post to the psilocybin. In some embodiments, the serotonin receptor modulator is nelotanserin and the psychedelic is psilocybin, wherein the nelotanserin is administered to post-treat at least 270 minutes post to the psilocybin. In some embodiments, the serotonin receptor modulator is nelotanserin and the psychedelic is psilocybin, wherein the nelotanserin is administered to post-treat at least 300 minutes post to the psilocybin. In some embodiments, the serotonin receptor modulator is nelotanserin and the psychedelic is psilocybin, wherein the nelotanserin is administered to post-treat at least 330 minutes post to the psilocybin. In some embodiments, the serotonin receptor modulator is nelotanserin and the psychedelic is psilocybin, wherein the nelotanserin is administered to post-treat at least 360 minutes post to the psilocybin. In some preferred embodiments, the serotonin receptor modulator is nelotanserin and the psychedelic is psilocybin, wherein nelotanserin is administered to post-treat between about 60 minutes and about 180 minutes post to the administration of psilocybin.

In some embodiments, the serotonin receptor modulator is pruvanserin and the psychedelic is psilocybin, wherein the pruvanserin is administered to post-treat at least 15 minutes post to the administration of psilocybin. In some embodiments, the serotonin receptor modulator is pruvanserin and the psychedelic is psilocybin, wherein the pruvanserin is administered to post-treat at least 30 minutes post to the psilocybin. In some embodiments, the serotonin receptor modulator is pruvanserin and the psychedelic is psilocybin, wherein the pruvanserin is administered to post-treat at least 60 minutes post to the psilocybin. In some embodiments, the serotonin receptor modulator is pruvanserin and the psychedelic is psilocybin, wherein the pruvanserin is administered to post-treat at least 90 minutes post to the psilocybin. In some embodiments, the serotonin receptor modulator is pruvanserin and the psychedelic is psilocybin, wherein the pruvanserin is administered to post-treat at least 120 minutes post to the psilocybin. In some embodiments, the serotonin receptor modulator is pruvanserin and the psychedelic is psilocybin, wherein the pruvanserin is administered to post-treat at least 150 minutes post to the psilocybin. In some embodiments, the serotonin receptor modulator is pruvanserin and the psychedelic is psilocybin, wherein the pruvanserin is administered to post-treat between about 15 minutes and about 150 minutes post to the psilocybin. In some embodiments, the serotonin receptor modulator is pruvanserin and the psychedelic is psilocybin, wherein the pruvanserin is administered to post-treat at least 180 minutes post to the psilocybin. In some embodiments, the serotonin receptor modulator is pruvanserin and the psychedelic is psilocybin, wherein the pruvanserin is administered to post-treat at least 210 minutes post to the psilocybin. In some embodiments, the serotonin receptor modulator is pruvanserin and the psychedelic is psilocybin, wherein the pruvanserin is administered to post-treat at least 240 minutes post to the psilocybin. In some embodiments, the serotonin receptor modulator is pruvanserin and the psychedelic is psilocybin, wherein the pruvanserin is administered to post-treat at least 270 minutes post to the psilocybin. In some embodiments, the serotonin receptor modulator is pruvanserin and the psychedelic is psilocybin, wherein the pruvanserin is administered to post-treat at least 300 minutes post to the psilocybin. In some embodiments, the serotonin receptor modulator is pruvanserin and the psychedelic is psilocybin, wherein the pruvanserin is administered to post-treat at least 330 minutes post to the psilocybin. In some embodiments, the serotonin receptor modulator is pruvanserin and the psychedelic is psilocybin, wherein the pruvanserin is administered to post-treat at least 360 minutes post to the psilocybin. In some preferred embodiments, the serotonin receptor modulator is pruvanserin and the psychedelic is psilocybin, wherein pruvanserin is administered to post-treat between about 60 minutes and about 180 minutes post to the administration of psilocybin.

In some embodiments, the serotonin receptor modulator is flibanserin and the psychedelic is psilocybin, wherein the flibanserin is administered to post-treat at least 15 minutes post to the administration of psilocybin. In some embodiments, the serotonin receptor modulator is flibanserin and the psychedelic is psilocybin, wherein the flibanserin is administered to post-treat at least 30 minutes post to the psilocybin. In some embodiments, the serotonin receptor modulator is flibanserin and the psychedelic is psilocybin, wherein the flibanserin is administered to post-treat at least 60 minutes post to the psilocybin. In some embodiments, the serotonin receptor modulator is flibanserin and the psychedelic is psilocybin, wherein the flibanserin is administered to post-treat at least 90 minutes post to the psilocybin. In some embodiments, the serotonin receptor modulator is flibanserin and the psychedelic is psilocybin, wherein the flibanserin is administered to post-treat at least 120 minutes post to the psilocybin. In some embodiments, the serotonin receptor modulator is flibanserin and the psychedelic is psilocybin, wherein the flibanserin is administered to post-treat at least 150 minutes post to the psilocybin. In some embodiments, the serotonin receptor modulator is flibanserin and the psychedelic is psilocybin, wherein the flibanserin is administered to post-treat between about 15 minutes and about 150 minutes post to the psilocybin. In some embodiments, the serotonin receptor modulator is flibanserin and the psychedelic is psilocybin, wherein the flibanserin is administered to post-treat at least 180 minutes post to the psilocybin. In some embodiments, the serotonin receptor modulator is flibanserin and the psychedelic is psilocybin, wherein the flibanserin is administered to post-treat at least 210 minutes post to the psilocybin. In some embodiments, the serotonin receptor modulator is flibanserin and the psychedelic is psilocybin, wherein the flibanserin is administered to post-treat at least 240 minutes post to the psilocybin. In some embodiments, the serotonin receptor modulator is flibanserin and the psychedelic is psilocybin, wherein the flibanserin is administered to post-treat at least 270 minutes post to the psilocybin. In some embodiments, the serotonin receptor modulator is flibanserin and the psychedelic is psilocybin, wherein the flibanserin is administered to post-treat at least 300 minutes post to the psilocybin. In some embodiments, the serotonin receptor modulator is flibanserin and the psychedelic is psilocybin, wherein the flibanserin is administered to post-treat at least 330 minutes post to the psilocybin. In some embodiments, the serotonin receptor modulator is flibanserin and the psychedelic is psilocybin, wherein the flibanserin is administered to post-treat at least 360 minutes post to the psilocybin. In some preferred embodiments, the serotonin receptor modulator is flibanserin and the psychedelic is psilocybin, wherein flibanserin is administered to post-treat between about 60 minutes and about 180 minutes post to the administration of psilocybin.

In some embodiments, the serotonin receptor modulator is olanzapine and the psychedelic is psilocybin, wherein the olanzapine is administered to post-treat at least 15 minutes post to the administration of psilocybin. In some embodiments, the serotonin receptor modulator is olanzapine and the psychedelic is psilocybin, wherein the olanzapine is administered to post-treat at least 30 minutes post to the psilocybin. In some embodiments, the serotonin receptor modulator is olanzapine and the psychedelic is psilocybin, wherein the olanzapine is administered to post-treat at least 60 minutes post to the psilocybin. In some embodiments, the serotonin receptor modulator is olanzapine and the psychedelic is psilocybin, wherein the olanzapine is administered to post-treat at least 90 minutes post to the psilocybin. In some embodiments, the serotonin receptor modulator is olanzapine and the psychedelic is psilocybin, wherein the olanzapine is administered to post-treat at least 120 minutes post to the psilocybin. In some embodiments, the serotonin receptor modulator is olanzapine and the psychedelic is psilocybin, wherein the olanzapine is administered to post-treat at least 150 minutes post to the psilocybin. In some embodiments, the serotonin receptor modulator is olanzapine and the psychedelic is psilocybin, wherein the olanzapine is administered to post-treat between about 15 minutes and about 150 minutes post to the psilocybin. In some embodiments, the serotonin receptor modulator is olanzapine and the psychedelic is psilocybin, wherein the olanzapine is administered to post-treat at least 180 minutes post to the psilocybin. In some embodiments, the serotonin receptor modulator is olanzapine and the psychedelic is psilocybin, wherein the olanzapine is administered to post-treat at least 210 minutes post to the psilocybin. In some embodiments, the serotonin receptor modulator is olanzapine and the psychedelic is psilocybin, wherein the olanzapine is administered to post-treat at least 240 minutes post to the psilocybin. In some embodiments, the serotonin receptor modulator is olanzapine and the psychedelic is psilocybin, wherein the olanzapine is administered to post-treat at least 270 minutes post to the psilocybin. In some embodiments, the serotonin receptor modulator is olanzapine and the psychedelic is psilocybin, wherein the olanzapine is administered to post-treat at least 300 minutes post to the psilocybin. In some embodiments, the serotonin receptor modulator is olanzapine and the psychedelic is psilocybin, wherein the olanzapine is administered to post-treat at least 330 minutes post to the psilocybin. In some embodiments, the serotonin receptor modulator is olanzapine and the psychedelic is psilocybin, wherein the olanzapine is administered to post-treat at least 360 minutes post to the psilocybin. In some preferred embodiments, the serotonin receptor modulator is olanzapine and the psychedelic is psilocybin, wherein olanzapine is administered to post-treat between about 60 minutes and about 180 minutes post to the administration of psilocybin.

In some embodiments, the serotonin receptor modulator is quetiapine and the psychedelic is psilocybin, wherein the quetiapine is administered to post-treat at least 15 minutes post to the administration of psilocybin. In some embodiments, the serotonin receptor modulator is quetiapine and the psychedelic is psilocybin, wherein the quetiapine is administered to post-treat at least 30 minutes post to the psilocybin. In some embodiments, the serotonin receptor modulator is quetiapine and the psychedelic is psilocybin, wherein the quetiapine is administered to post-treat at least 60 minutes post to the psilocybin. In some embodiments, the serotonin receptor modulator is quetiapine and the psychedelic is psilocybin, wherein the quetiapine is administered to post-treat at least 90 minutes post to the psilocybin. In some embodiments, the serotonin receptor modulator is quetiapine and the psychedelic is psilocybin, wherein the quetiapine is administered to post-treat at least 120 minutes post to the psilocybin. In some embodiments, the serotonin receptor modulator is quetiapine and the psychedelic is psilocybin, wherein the quetiapine is administered to post-treat at least 150 minutes post to the psilocybin. In some embodiments, the serotonin receptor modulator is quetiapine and the psychedelic is psilocybin, wherein the quetiapine is administered to post-treat between about 15 minutes and about 150 minutes post to the psilocybin. In some embodiments, the serotonin receptor modulator is quetiapine and the psychedelic is psilocybin, wherein the quetiapine is administered to post-treat at least 180 minutes post to the psilocybin. In some embodiments, the serotonin receptor modulator is quetiapine and the psychedelic is psilocybin, wherein the quetiapine is administered to post-treat at least 210 minutes post to the psilocybin. In some embodiments, the serotonin receptor modulator is quetiapine and the psychedelic is psilocybin, wherein the quetiapine is administered to post-treat at least 240 minutes post to the psilocybin. In some embodiments, the serotonin receptor modulator is quetiapine and the psychedelic is psilocybin, wherein the quetiapine is administered to post-treat at least 270 minutes post to the psilocybin. In some embodiments, the serotonin receptor modulator is quetiapine and the psychedelic is psilocybin, wherein the quetiapine is administered to post-treat at least 300 minutes post to the psilocybin. In some embodiments, the serotonin receptor modulator is quetiapine and the psychedelic is psilocybin, wherein the quetiapine is administered to post-treat at least 330 minutes post to the psilocybin. In some embodiments, the serotonin receptor modulator is quetiapine and the psychedelic is psilocybin, wherein the quetiapine is administered to post-treat at least 360 minutes post to the psilocybin. In some preferred embodiments, the serotonin receptor modulator is quetiapine and the psychedelic is psilocybin, wherein quetiapine is administered to post-treat between about 60 minutes and about 180 minutes post to the administration of psilocybin.

In some embodiments, the serotonin receptor modulator is risperidone and the psychedelic is psilocybin, wherein the risperidone is administered to post-treat at least 15 minutes post to the administration of psilocybin. In some embodiments, the serotonin receptor modulator is risperidone and the psychedelic is psilocybin, wherein the risperidone is administered to post-treat at least 30 minutes post to the psilocybin. In some embodiments, the serotonin receptor modulator is risperidone and the psychedelic is psilocybin, wherein the risperidone is administered to post-treat at least 60 minutes post to the psilocybin. In some embodiments, the serotonin receptor modulator is risperidone and the psychedelic is psilocybin, wherein the risperidone is administered to post-treat at least 90 minutes post to the psilocybin. In some embodiments, the serotonin receptor modulator is risperidone and the psychedelic is psilocybin, wherein the risperidone is administered to post-treat at least 120 minutes post to the psilocybin. In some embodiments, the serotonin receptor modulator is risperidone and the psychedelic is psilocybin, wherein the risperidone is administered to post-treat at least 150 minutes post to the psilocybin. In some embodiments, the serotonin receptor modulator is risperidone and the psychedelic is psilocybin, wherein the risperidone is administered to post-treat between about 15 minutes and about 150 minutes post to the psilocybin. In some embodiments, the serotonin receptor modulator is risperidone and the psychedelic is psilocybin, wherein the risperidone is administered to post-treat at least 180 minutes post to the psilocybin. In some embodiments, the serotonin receptor modulator is risperidone and the psychedelic is psilocybin, wherein the risperidone is administered to post-treat at least 210 minutes post to the psilocybin. In some embodiments, the serotonin receptor modulator is risperidone and the psychedelic is psilocybin, wherein the risperidone is administered to post-treat at least 240 minutes post to the psilocybin. In some embodiments, the serotonin receptor modulator is risperidone and the psychedelic is psilocybin, wherein the risperidone is administered to post-treat at least 270 minutes post to the psilocybin. In some embodiments, the serotonin receptor modulator is risperidone and the psychedelic is psilocybin, wherein the risperidone is administered to post-treat at least 300 minutes post to the psilocybin. In some embodiments, the serotonin receptor modulator is risperidone and the psychedelic is psilocybin, wherein the risperidone is administered to post-treat at least 330 minutes post to the psilocybin. In some embodiments, the serotonin receptor modulator is risperidone and the psychedelic is psilocybin, wherein the risperidone is administered to post-treat at least 360 minutes post to the psilocybin. In some preferred embodiments, the serotonin receptor modulator is risperidone and the psychedelic is psilocybin, wherein risperidone is administered to post-treat between about 60 minutes and about 180 minutes post to the administration of psilocybin.

In some embodiments, the serotonin receptor modulator is eplivanserin and the psychedelic is 4-Acetoxy-DMT, wherein the eplivanserin is administered to post-treat at least 15 minutes post to the administration of 4-Acetoxy-DMT. In some embodiments, the serotonin receptor modulator is eplivanserin and the psychedelic is 4-Acetoxy-DMT, wherein the eplivanserin is administered to post-treat at least 30 minutes post to the 4-Acetoxy-DMT. In some embodiments, the serotonin receptor modulator is eplivanserin and the psychedelic is 4-Acetoxy-DMT, wherein the eplivanserin is administered to post-treat at least 60 minutes post to the 4-Acetoxy-DMT. In some embodiments, the serotonin receptor modulator is eplivanserin and the psychedelic is 4-Acetoxy-DMT, wherein the eplivanserin is administered to post-treat at least 90 minutes post to the 4-Acetoxy-DMT. In some embodiments, the serotonin receptor modulator is eplivanserin and the psychedelic is 4-Acetoxy-DMT, wherein the eplivanserin is administered to post-treat at least 120 minutes post to the 4-Acetoxy-DMT. In some embodiments, the serotonin receptor modulator is eplivanserin and the psychedelic is 4-Acetoxy-DMT, wherein the eplivanserin is administered to post-treat at least 150 minutes post to the 4-Acetoxy-DMT. In some embodiments, the serotonin receptor modulator is eplivanserin and the psychedelic is 4-Acetoxy-DMT, wherein the eplivanserin is administered to post-treat between about 15 minutes and about 150 minutes post to the 4-Acetoxy-DMT. In some embodiments, the serotonin receptor modulator is eplivanserin and the psychedelic is 4-Acetoxy-DMT, wherein the eplivanserin is administered to post-treat at least 180 minutes post to the 4-Acetoxy-DMT. In some embodiments, the serotonin receptor modulator is eplivanserin and the psychedelic is 4-Acetoxy-DMT, wherein the eplivanserin is administered to post-treat at least 210 minutes post to the 4-Acetoxy-DMT. In some embodiments, the serotonin receptor modulator is eplivanserin and the psychedelic is 4-Acetoxy-DMT, wherein the eplivanserin is administered to post-treat at least 240 minutes post to the 4-Acetoxy-DMT. In some embodiments, the serotonin receptor modulator is eplivanserin and the psychedelic is 4-Acetoxy-DMT, wherein the eplivanserin is administered to post-treat at least 270 minutes post to the 4-Acetoxy-DMT. In some embodiments, the serotonin receptor modulator is eplivanserin and the psychedelic is 4-Acetoxy-DMT, wherein the eplivanserin is administered to post-treat at least 300 minutes post to the 4-Acetoxy-DMT. In some embodiments, the serotonin receptor modulator is eplivanserin and the psychedelic is 4-Acetoxy-DMT, wherein the eplivanserin is administered to post-treat at least 330 minutes post to the 4-Acetoxy-DMT. In some embodiments, the serotonin receptor modulator is eplivanserin and the psychedelic is 4-Acetoxy-DMT, wherein the eplivanserin is administered to post-treat at least 360 minutes post to the 4-Acetoxy-DMT.

In some preferred embodiments, the serotonin receptor modulator is eplivanserin and the psychedelic is 4-Acetoxy-DMT, wherein eplivanserin is administered to post-treat between about 60 minutes and about 180 minutes post to the administration of 4-Acetoxy-DMT.

In some embodiments, the serotonin receptor modulator is volinanserin and the psychedelic is 4-Acetoxy-DMT, wherein the volinanserin is administered to post-treat at least 15 minutes post to the administration of 4-Acetoxy-DMT. In some embodiments, the serotonin receptor modulator is volinanserin and the psychedelic is 4-Acetoxy-DMT, wherein the volinanserin is administered to post-treat at least 30 minutes post to the 4-Acetoxy-DMT. In some embodiments, the serotonin receptor modulator is volinanserin and the psychedelic is 4-Acetoxy-DMT, wherein the volinanserin is administered to post-treat at least 60 minutes post to the 4-Acetoxy-DMT. In some embodiments, the serotonin receptor modulator is volinanserin and the psychedelic is 4-Acetoxy-DMT, wherein the volinanserin is administered to post-treat at least 90 minutes post to the 4-Acetoxy-DMT. In some embodiments, the serotonin receptor modulator is volinanserin and the psychedelic is 4-Acetoxy-DMT, wherein the volinanserin is administered to post-treat at least 120 minutes post to the 4-Acetoxy-DMT. In some embodiments, the serotonin receptor modulator is volinanserin and the psychedelic is 4-Acetoxy-DMT, wherein the volinanserin is administered to post-treat at least 150 minutes post to the 4-Acetoxy-DMT. In some embodiments, the serotonin receptor modulator is volinanserin and the psychedelic is 4-Acetoxy-DMT, wherein the volinanserin is administered to post-treat between about 15 minutes and about 150 minutes post to the 4-Acetoxy-DMT. In some embodiments, the serotonin receptor modulator is volinanserin and the psychedelic is 4-Acetoxy-DMT, wherein the volinanserin is administered to post-treat at least 180 minutes post to the 4-Acetoxy-DMT. In some embodiments, the serotonin receptor modulator is volinanserin and the psychedelic is 4-Acetoxy-DMT, wherein the volinanserin is administered to post-treat at least 210 minutes post to the 4-Acetoxy-DMT. In some embodiments, the serotonin receptor modulator is volinanserin and the psychedelic is 4-Acetoxy-DMT, wherein the volinanserin is administered to post-treat at least 240 minutes post to the 4-Acetoxy-DMT. In some embodiments, the serotonin receptor modulator is volinanserin and the psychedelic is 4-Acetoxy-DMT, wherein the volinanserin is administered to post-treat at least 270 minutes post to the 4-Acetoxy-DMT. In some embodiments, the serotonin receptor modulator is volinanserin and the psychedelic is 4-Acetoxy-DMT, wherein the volinanserin is administered to post-treat at least 300 minutes post to the 4-Acetoxy-DMT. In some embodiments, the serotonin receptor modulator is volinanserin and the psychedelic is 4-Acetoxy-DMT, wherein the volinanserin is administered to post-treat at least 330 minutes post to the 4-Acetoxy-DMT. In some embodiments, the serotonin receptor modulator is volinanserin and the psychedelic is 4-Acetoxy-DMT, wherein the volinanserin is administered to post-treat at least 360 minutes post to the 4-Acetoxy-DMT. In some preferred embodiments, the serotonin receptor modulator is volinanserin and the psychedelic is 4-Acetoxy-DMT, wherein volinanserin is administered to post-treat between about 60 minutes and about 180 minutes post to the administration of 4-Acetoxy-DMT.

In some embodiments, the serotonin receptor modulator is ketanserin and the psychedelic is 4-Acetoxy-DMT, wherein the ketanserin is administered to post-treat at least 15 minutes post to the administration of 4-Acetoxy-DMT. In some embodiments, the serotonin receptor modulator is ketanserin and the psychedelic is 4-Acetoxy-DMT, wherein the ketanserin is administered to post-treat at least 30 minutes post to the 4-Acetoxy-DMT. In some embodiments, the serotonin receptor modulator is ketanserin and the psychedelic is 4-Acetoxy-DMT, wherein the ketanserin is administered to post-treat at least 60 minutes post to the 4-Acetoxy-DMT. In some embodiments, the serotonin receptor modulator is ketanserin and the psychedelic is 4-Acetoxy-DMT, wherein the ketanserin is administered to post-treat at least 90 minutes post to the 4-Acetoxy-DMT. In some embodiments, the serotonin receptor modulator is ketanserin and the psychedelic is 4-Acetoxy-DMT, wherein the ketanserin is administered to post-treat at least 120 minutes post to the 4-Acetoxy-DMT. In some embodiments, the serotonin receptor modulator is ketanserin and the psychedelic is 4-Acetoxy-DMT, wherein the ketanserin is administered to post-treat at least 150 minutes post to the 4-Acetoxy-DMT. In some embodiments, the serotonin receptor modulator is ketanserin and the psychedelic is 4-Acetoxy-DMT, wherein the ketanserin is administered to post-treat between about 15 minutes and about 150 minutes post to the 4-Acetoxy-DMT. In some embodiments, the serotonin receptor modulator is ketanserin and the psychedelic is 4-Acetoxy-DMT, wherein the ketanserin is administered to post-treat at least 180 minutes post to the 4-Acetoxy-DMT. In some embodiments, the serotonin receptor modulator is ketanserin and the psychedelic is 4-Acetoxy-DMT, wherein the ketanserin is administered to post-treat at least 210 minutes post to the 4-Acetoxy-DMT. In some embodiments, the serotonin receptor modulator is ketanserin and the psychedelic is 4-Acetoxy-DMT, wherein the ketanserin is administered to post-treat at least 240 minutes post to the 4-Acetoxy-DMT. In some embodiments, the serotonin receptor modulator is ketanserin and the psychedelic is 4-Acetoxy-DMT, wherein the ketanserin is administered to post-treat at least 270 minutes post to the 4-Acetoxy-DMT. In some embodiments, the serotonin receptor modulator is ketanserin and the psychedelic is 4-Acetoxy-DMT, wherein the ketanserin is administered to post-treat at least 300 minutes post to the 4-Acetoxy-DMT. In some embodiments, the serotonin receptor modulator is ketanserin and the psychedelic is 4-Acetoxy-DMT, wherein the ketanserin is administered to post-treat at least 330 minutes post to the 4-Acetoxy-DMT. In some embodiments, the serotonin receptor modulator is ketanserin and the psychedelic is 4-Acetoxy-DMT, wherein the ketanserin is administered to post-treat at least 360 minutes post to the 4-Acetoxy-DMT. In some preferred embodiments, the serotonin receptor modulator is ketanserin and the psychedelic is 4-Acetoxy-DMT, wherein ketanserin is administered to post-treat between about 60 minutes and about 180 minutes post to the administration of 4-Acetoxy-DMT.

In some embodiments, the serotonin receptor modulator is ritanserin and the psychedelic is 4-Acetoxy-DMT, wherein the ritanserin is administered to post-treat at least 15 minutes post to the administration of 4-Acetoxy-DMT. In some embodiments, the serotonin receptor modulator is ritanserin and the psychedelic is 4-Acetoxy-DMT, wherein the ritanserin is administered to post-treat at least 30 minutes post to the 4-Acetoxy-DMT. In some embodiments, the serotonin receptor modulator is ritanserin and the psychedelic is 4-Acetoxy-DMT, wherein the ritanserin is administered to post-treat at least 60 minutes post to the 4-Acetoxy-DMT. In some embodiments, the serotonin receptor modulator is ritanserin and the psychedelic is 4-Acetoxy-DMT, wherein the ritanserin is administered to post-treat at least 90 minutes post to the 4-Acetoxy-DMT. In some embodiments, the serotonin receptor modulator is ritanserin and the psychedelic is 4-Acetoxy-DMT, wherein the ritanserin is administered to post-treat at least 120 minutes post to the 4-Acetoxy-DMT. In some embodiments, the serotonin receptor modulator is ritanserin and the psychedelic is 4-Acetoxy-DMT, wherein the ritanserin is administered to post-treat at least 150 minutes post to the 4-Acetoxy-DMT. In some embodiments, the serotonin receptor modulator is ritanserin and the psychedelic is 4-Acetoxy-DMT, wherein the ritanserin is administered to post-treat between about 15 minutes and about 150 minutes post to the 4-Acetoxy-DMT. In some embodiments, the serotonin receptor modulator is ritanserin and the psychedelic is 4-Acetoxy-DMT, wherein the ritanserin is administered to post-treat at least 180 minutes post to the 4-Acetoxy-DMT. In some embodiments, the serotonin receptor modulator is ritanserin and the psychedelic is 4-Acetoxy-DMT, wherein the ritanserin is administered to post-treat at least 210 minutes post to the 4-Acetoxy-DMT. In some embodiments, the serotonin receptor modulator is ritanserin and the psychedelic is 4-Acetoxy-DMT, wherein the ritanserin is administered to post-treat at least 240 minutes post to the 4-Acetoxy-DMT. In some embodiments, the serotonin receptor modulator is ritanserin and the psychedelic is 4-Acetoxy-DMT, wherein the ritanserin is administered to post-treat at least 270 minutes post to the 4-Acetoxy-DMT. In some embodiments, the serotonin receptor modulator is ritanserin and the psychedelic is 4-Acetoxy-DMT, wherein the ritanserin is administered to post-treat at least 300 minutes post to the 4-Acetoxy-DMT. In some embodiments, the serotonin receptor modulator is ritanserin and the psychedelic is 4-Acetoxy-DMT, wherein the ritanserin is administered to post-treat at least 330 minutes post to the 4-Acetoxy-DMT. In some embodiments, the serotonin receptor modulator is ritanserin and the psychedelic is 4-Acetoxy-DMT, wherein the ritanserin is administered to post-treat at least 360 minutes post to the 4-Acetoxy-DMT. In some preferred embodiments, the serotonin receptor modulator is ritanserin and the psychedelic is 4-Acetoxy-DMT, wherein ritanserin is administered to post-treat between about 60 minutes and about 180 minutes post to the administration of 4-Acetoxy-DMT.

In some embodiments, the serotonin receptor modulator is pimavanserin and the psychedelic is 4-Acetoxy-DMT, wherein the pimavanserin is administered to post-treat at least 15 minutes post to the administration of 4-Acetoxy-DMT. In some embodiments, the serotonin receptor modulator is pimavanserin and the psychedelic is 4-Acetoxy-DMT, wherein the pimavanserin is administered to post-treat at least 30 minutes post to the 4-Acetoxy-DMT. In some embodiments, the serotonin receptor modulator is pimavanserin and the psychedelic is 4-Acetoxy-DMT, wherein the pimavanserin is administered to post-treat at least 60 minutes post to the 4-Acetoxy-DMT. In some embodiments, the serotonin receptor modulator is pimavanserin and the psychedelic is 4-Acetoxy-DMT, wherein the pimavanserin is administered to post-treat at least 90 minutes post to the 4-Acetoxy-DMT. In some embodiments, the serotonin receptor modulator is pimavanserin and the psychedelic is 4-Acetoxy-DMT, wherein the pimavanserin is administered to post-treat at least 120 minutes post to the 4-Acetoxy-DMT. In some embodiments, the serotonin receptor modulator is pimavanserin and the psychedelic is 4-Acetoxy-DMT, wherein the pimavanserin is administered to post-treat at least 150 minutes post to the 4-Acetoxy-DMT. In some embodiments, the serotonin receptor modulator is pimavanserin and the psychedelic is 4-Acetoxy-DMT, wherein the pimavanserin is administered to post-treat between about 15 minutes and about 150 minutes post to the 4-Acetoxy-DMT. In some embodiments, the serotonin receptor modulator is pimavanserin and the psychedelic is 4-Acetoxy-DMT, wherein the pimavanserin is administered to post-treat at least 180 minutes post to the 4-Acetoxy-DMT. In some embodiments, the serotonin receptor modulator is pimavanserin and the psychedelic is 4-Acetoxy-DMT, wherein the pimavanserin is administered to post-treat at least 210 minutes post to the 4-Acetoxy-DMT. In some embodiments, the serotonin receptor modulator is pimavanserin and the psychedelic is 4-Acetoxy-DMT, wherein the pimavanserin is administered to post-treat at least 240 minutes post to the 4-Acetoxy-DMT. In some embodiments, the serotonin receptor modulator is pimavanserin and the psychedelic is 4-Acetoxy-DMT, wherein the pimavanserin is administered to post-treat at least 270 minutes post to the 4-Acetoxy-DMT. In some embodiments, the serotonin receptor modulator is pimavanserin and the psychedelic is 4-Acetoxy-DMT, wherein the pimavanserin is administered to post-treat at least 300 minutes post to the 4-Acetoxy-DMT. In some embodiments, the serotonin receptor modulator is pimavanserin and the psychedelic is 4-Acetoxy-DMT, wherein the pimavanserin is administered to post-treat at least 330 minutes post to the 4-Acetoxy-DMT. In some embodiments, the serotonin receptor modulator is pimavanserin and the psychedelic is 4-Acetoxy-DMT, wherein the pimavanserin is administered to post-treat at least 360 minutes post to the 4-Acetoxy-DMT. In some preferred embodiments, the serotonin receptor modulator is pimavanserin and the psychedelic is 4-Acetoxy-DMT, wherein pimavanserin is administered to post-treat between about 60 minutes and about 180 minutes post to the administration of 4-Acetoxy-DMT.

In some embodiments, the serotonin receptor modulator is nelotanserin and the psychedelic is 4-Acetoxy-DMT, wherein the nelotanserin is administered to post-treat at least 15 minutes post to the administration of 4-Acetoxy-DMT. In some embodiments, the serotonin receptor modulator is nelotanserin and the psychedelic is 4-Acetoxy-DMT, wherein the nelotanserin is administered to post-treat at least 30 minutes post to the 4-Acetoxy-DMT. In some embodiments, the serotonin receptor modulator is nelotanserin and the psychedelic is 4-Acetoxy-DMT, wherein the nelotanserin is administered to post-treat at least 60 minutes post to the 4-Acetoxy-DMT. In some embodiments, the serotonin receptor modulator is nelotanserin and the psychedelic is 4-Acetoxy-DMT, wherein the nelotanserin is administered to post-treat at least 90 minutes post to the 4-Acetoxy-DMT. In some embodiments, the serotonin receptor modulator is nelotanserin and the psychedelic is 4-Acetoxy-DMT, wherein the nelotanserin is administered to post-treat at least 120 minutes post to the 4-Acetoxy-DMT. In some embodiments, the serotonin receptor modulator is nelotanserin and the psychedelic is 4-Acetoxy-DMT, wherein the nelotanserin is administered to post-treat at least 150 minutes post to the 4-Acetoxy-DMT. In some embodiments, the serotonin receptor modulator is nelotanserin and the psychedelic is 4-Acetoxy-DMT, wherein the nelotanserin is administered to post-treat between about 15 minutes and about 150 minutes post to the 4-Acetoxy-DMT. In some embodiments, the serotonin receptor modulator is nelotanserin and the psychedelic is 4-Acetoxy-DMT, wherein the nelotanserin is administered to post-treat at least 180 minutes post to the 4-Acetoxy-DMT. In some embodiments, the serotonin receptor modulator is nelotanserin and the psychedelic is 4-Acetoxy-DMT, wherein the nelotanserin is administered to post-treat at least 210 minutes post to the 4-Acetoxy-DMT. In some embodiments, the serotonin receptor modulator is nelotanserin and the psychedelic is 4-Acetoxy-DMT, wherein the nelotanserin is administered to post-treat at least 240 minutes post to the 4-Acetoxy-DMT. In some embodiments, the serotonin receptor modulator is nelotanserin and the psychedelic is 4-Acetoxy-DMT, wherein the nelotanserin is administered to post-treat at least 270 minutes post to the 4-Acetoxy-DMT. In some embodiments, the serotonin receptor modulator is nelotanserin and the psychedelic is 4-Acetoxy-DMT, wherein the nelotanserin is administered to post-treat at least 300 minutes post to the 4-Acetoxy-DMT. In some embodiments, the serotonin receptor modulator is nelotanserin and the psychedelic is 4-Acetoxy-DMT, wherein the nelotanserin is administered to post-treat at least 330 minutes post to the 4-Acetoxy-DMT. In some embodiments, the serotonin receptor modulator is nelotanserin and the psychedelic is 4-Acetoxy-DMT, wherein the nelotanserin is administered to post-treat at least 360 minutes post to the 4-Acetoxy-DMT. In some preferred embodiments, the serotonin receptor modulator is nelotanserin and the psychedelic is 4-Acetoxy-DMT, wherein nelotanserin is administered to post-treat between about 60 minutes and about 180 minutes post to the administration of 4-Acetoxy-DMT.

In some embodiments, the serotonin receptor modulator is pruvanserin and the psychedelic is 4-Acetoxy-DMT, wherein the pruvanserin is administered to post-treat at least 15 minutes post to the administration of 4-Acetoxy-DMT. In some embodiments, the serotonin receptor modulator is pruvanserin and the psychedelic is 4-Acetoxy-DMT, wherein the pruvanserin is administered to post-treat at least 30 minutes post to the 4-Acetoxy-DMT. In some embodiments, the serotonin receptor modulator is pruvanserin and the psychedelic is 4-Acetoxy-DMT, wherein the pruvanserin is administered to post-treat at least 60 minutes post to the 4-Acetoxy-DMT. In some embodiments, the serotonin receptor modulator is pruvanserin and the psychedelic is 4-Acetoxy-DMT, wherein the pruvanserin is administered to post-treat at least 90 minutes post to the 4-Acetoxy-DMT. In some embodiments, the serotonin receptor modulator is pruvanserin and the psychedelic is 4-Acetoxy-DMT, wherein the pruvanserin is administered to post-treat at least 120 minutes post to the 4-Acetoxy-DMT. In some embodiments, the serotonin receptor modulator is pruvanserin and the psychedelic is 4-Acetoxy-DMT, wherein the pruvanserin is administered to post-treat at least 150 minutes post to the 4-Acetoxy-DMT. In some embodiments, the serotonin receptor modulator is pruvanserin and the psychedelic is 4-Acetoxy-DMT, wherein the pruvanserin is administered to post-treat between about 15 minutes and about 150 minutes post to the 4-Acetoxy-DMT. In some embodiments, the serotonin receptor modulator is pruvanserin and the psychedelic is 4-Acetoxy-DMT, wherein the pruvanserin is administered to post-treat at least 180 minutes post to the 4-Acetoxy-DMT. In some embodiments, the serotonin receptor modulator is pruvanserin and the psychedelic is 4-Acetoxy-DMT, wherein the pruvanserin is administered to post-treat at least 210 minutes post to the 4-Acetoxy-DMT. In some embodiments, the serotonin receptor modulator is pruvanserin and the psychedelic is 4-Acetoxy-DMT, wherein the pruvanserin is administered to post-treat at least 240 minutes post to the 4-Acetoxy-DMT. In some embodiments, the serotonin receptor modulator is pruvanserin and the psychedelic is 4-Acetoxy-DMT, wherein the pruvanserin is administered to post-treat at least 270 minutes post to the 4-Acetoxy-DMT. In some embodiments, the serotonin receptor modulator is pruvanserin and the psychedelic is 4-Acetoxy-DMT, wherein the pruvanserin is administered to post-treat at least 300 minutes post to the 4-Acetoxy-DMT. In some embodiments, the serotonin receptor modulator is pruvanserin and the psychedelic is 4-Acetoxy-DMT, wherein the pruvanserin is administered to post-treat at least 330 minutes post to the 4-Acetoxy-DMT. In some embodiments, the serotonin receptor modulator is pruvanserin and the psychedelic is 4-Acetoxy-DMT, wherein the pruvanserin is administered to post-treat at least 360 minutes post to the 4-Acetoxy-DMT. In some preferred embodiments, the serotonin receptor modulator is pruvanserin and the psychedelic is 4-Acetoxy-DMT, wherein pruvanserin is administered to post-treat between about 60 minutes and about 180 minutes post to the administration of 4-Acetoxy-DMT.

In some embodiments, the serotonin receptor modulator is flibanserin and the psychedelic is 4-Acetoxy-DMT, wherein the flibanserin is administered to post-treat at least 15 minutes post to the administration of 4-Acetoxy-DMT. In some embodiments, the serotonin receptor modulator is flibanserin and the psychedelic is 4-Acetoxy-DMT, wherein the flibanserin is administered to post-treat at least 30 minutes post to the 4-Acetoxy-DMT. In some embodiments, the serotonin receptor modulator is flibanserin and the psychedelic is 4-Acetoxy-DMT, wherein the flibanserin is administered to post-treat at least 60 minutes post to the 4-Acetoxy-DMT. In some embodiments, the serotonin receptor modulator is flibanserin and the psychedelic is 4-Acetoxy-DMT, wherein the flibanserin is administered to post-treat at least 90 minutes post to the 4-Acetoxy-DMT. In some embodiments, the serotonin receptor modulator is flibanserin and the psychedelic is 4-Acetoxy-DMT, wherein the flibanserin is administered to post-treat at least 120 minutes post to the 4-Acetoxy-DMT. In some embodiments, the serotonin receptor modulator is flibanserin and the psychedelic is 4-Acetoxy-DMT, wherein the flibanserin is administered to post-treat at least 150 minutes post to the 4-Acetoxy-DMT. In some embodiments, the serotonin receptor modulator is flibanserin and the psychedelic is 4-Acetoxy-DMT, wherein the flibanserin is administered to post-treat between about 15 minutes and about 150 minutes post to the 4-Acetoxy-DMT. In some embodiments, the serotonin receptor modulator is flibanserin and the psychedelic is 4-Acetoxy-DMT, wherein the flibanserin is administered to post-treat at least 180 minutes post to the 4-Acetoxy-DMT. In some embodiments, the serotonin receptor modulator is flibanserin and the psychedelic is 4-Acetoxy-DMT, wherein the flibanserin is administered to post-treat at least 210 minutes post to the 4-Acetoxy-DMT. In some embodiments, the serotonin receptor modulator is flibanserin and the psychedelic is 4-Acetoxy-DMT, wherein the flibanserin is administered to post-treat at least 240 minutes post to the 4-Acetoxy-DMT. In some embodiments, the serotonin receptor modulator is flibanserin and the psychedelic is 4-Acetoxy-DMT, wherein the flibanserin is administered to post-treat at least 270 minutes post to the 4-Acetoxy-DMT. In some embodiments, the serotonin receptor modulator is flibanserin and the psychedelic is 4-Acetoxy-DMT, wherein the flibanserin is administered to post-treat at least 300 minutes post to the 4-Acetoxy-DMT. In some embodiments, the serotonin receptor modulator is flibanserin and the psychedelic is 4-Acetoxy-DMT, wherein the flibanserin is administered to post-treat at least 330 minutes post to the 4-Acetoxy-DMT. In some embodiments, the serotonin receptor modulator is flibanserin and the psychedelic is 4-Acetoxy-DMT, wherein the flibanserin is administered to post-treat at least 360 minutes post to the 4-Acetoxy-DMT. In some preferred embodiments, the serotonin receptor modulator is flibanserin and the psychedelic is 4-Acetoxy-DMT, wherein flibanserin is administered to post-treat between about 60 minutes and about 180 minutes post to the administration of 4-Acetoxy-DMT.

In some embodiments, the serotonin receptor modulator is olanzapine and the psychedelic is 4-Acetoxy-DMT, wherein the olanzapine is administered to post-treat at least 15 minutes post to the administration of 4-Acetoxy-DMT. In some embodiments, the serotonin receptor modulator is olanzapine and the psychedelic is 4-Acetoxy-DMT, wherein the olanzapine is administered to post-treat at least 30 minutes post to the 4-Acetoxy-DMT. In some embodiments, the serotonin receptor modulator is olanzapine and the psychedelic is 4-Acetoxy-DMT, wherein the olanzapine is administered to post-treat at least 60 minutes post to the 4-Acetoxy-DMT. In some embodiments, the serotonin receptor modulator is olanzapine and the psychedelic is 4-Acetoxy-DMT, wherein the olanzapine is administered to post-treat at least 90 minutes post to the 4-Acetoxy-DMT. In some embodiments, the serotonin receptor modulator is olanzapine and the psychedelic is 4-Acetoxy-DMT, wherein the olanzapine is administered to post-treat at least 120 minutes post to the 4-Acetoxy-DMT. In some embodiments, the serotonin receptor modulator is olanzapine and the psychedelic is 4-Acetoxy-DMT, wherein the olanzapine is administered to post-treat at least 150 minutes post to the 4-Acetoxy-DMT. In some embodiments, the serotonin receptor modulator is olanzapine and the psychedelic is 4-Acetoxy-DMT, wherein the olanzapine is administered to post-treat between about 15 minutes and about 150 minutes post to the 4-Acetoxy-DMT. In some embodiments, the serotonin receptor modulator is olanzapine and the psychedelic is 4-Acetoxy-DMT, wherein the olanzapine is administered to post-treat at least 180 minutes post to the 4-Acetoxy-DMT. In some embodiments, the serotonin receptor modulator is olanzapine and the psychedelic is 4-Acetoxy-DMT, wherein the olanzapine is administered to post-treat at least 210 minutes post to the 4-Acetoxy-DMT. In some embodiments, the serotonin receptor modulator is olanzapine and the psychedelic is 4-Acetoxy-DMT, wherein the olanzapine is administered to post-treat at least 240 minutes post to the 4-Acetoxy-DMT. In some embodiments, the serotonin receptor modulator is olanzapine and the psychedelic is 4-Acetoxy-DMT, wherein the olanzapine is administered to post-treat at least 270 minutes post to the 4-Acetoxy-DMT. In some embodiments, the serotonin receptor modulator is olanzapine and the psychedelic is 4-Acetoxy-DMT, wherein the olanzapine is administered to post-treat at least 300 minutes post to the 4-Acetoxy-DMT. In some embodiments, the serotonin receptor modulator is olanzapine and the psychedelic is 4-Acetoxy-DMT, wherein the olanzapine is administered to post-treat at least 330 minutes post to the 4-Acetoxy-DMT. In some embodiments, the serotonin receptor modulator is olanzapine and the psychedelic is 4-Acetoxy-DMT, wherein the olanzapine is administered to post-treat at least 360 minutes post to the 4-Acetoxy-DMT. In some preferred embodiments, the serotonin receptor modulator is olanzapine and the psychedelic is 4-Acetoxy-DMT, wherein olanzapine is administered to post-treat between about 60 minutes and about 180 minutes post to the administration of 4-Acetoxy-DMT.

In some embodiments, the serotonin receptor modulator is quetiapine and the psychedelic is 4-Acetoxy-DMT, wherein the quetiapine is administered to post-treat at least 15 minutes post to the administration of 4-Acetoxy-DMT. In some embodiments, the serotonin receptor modulator is quetiapine and the psychedelic is 4-Acetoxy-DMT, wherein the quetiapine is administered to post-treat at least 30 minutes post to the 4-Acetoxy-DMT. In some embodiments, the serotonin receptor modulator is quetiapine and the psychedelic is 4-Acetoxy-DMT, wherein the quetiapine is administered to post-treat at least 60 minutes post to the 4-Acetoxy-DMT. In some embodiments, the serotonin receptor modulator is quetiapine and the psychedelic is 4-Acetoxy-DMT, wherein the quetiapine is administered to post-treat at least 90 minutes post to the 4-Acetoxy-DMT. In some embodiments, the serotonin receptor modulator is quetiapine and the psychedelic is 4-Acetoxy-DMT, wherein the quetiapine is administered to post-treat at least 120 minutes post to the 4-Acetoxy-DMT. In some embodiments, the serotonin receptor modulator is quetiapine and the psychedelic is 4-Acetoxy-DMT, wherein the quetiapine is administered to post-treat at least 150 minutes post to the 4-Acetoxy-DMT. In some embodiments, the serotonin receptor modulator is quetiapine and the psychedelic is 4-Acetoxy-DMT, wherein the quetiapine is administered to post-treat between about 15 minutes and about 150 minutes post to the 4-Acetoxy-DMT. In some embodiments, the serotonin receptor modulator is quetiapine and the psychedelic is 4-Acetoxy-DMT, wherein the quetiapine is administered to post-treat at least 180 minutes post to the 4-Acetoxy-DMT. In some embodiments, the serotonin receptor modulator is quetiapine and the psychedelic is 4-Acetoxy-DMT, wherein the quetiapine is administered to post-treat at least 210 minutes post to the 4-Acetoxy-DMT. In some embodiments, the serotonin receptor modulator is quetiapine and the psychedelic is 4-Acetoxy-DMT, wherein the quetiapine is administered to post-treat at least 240 minutes post to the 4-Acetoxy-DMT. In some embodiments, the serotonin receptor modulator is quetiapine and the psychedelic is 4-Acetoxy-DMT, wherein the quetiapine is administered to post-treat at least 270 minutes post to the 4-Acetoxy-DMT. In some embodiments, the serotonin receptor modulator is quetiapine and the psychedelic is 4-Acetoxy-DMT, wherein the quetiapine is administered to post-treat at least 300 minutes post to the 4-Acetoxy-DMT. In some embodiments, the serotonin receptor modulator is quetiapine and the psychedelic is 4-Acetoxy-DMT, wherein the quetiapine is administered to post-treat at least 330 minutes post to the 4-Acetoxy-DMT. In some embodiments, the serotonin receptor modulator is quetiapine and the psychedelic is 4-Acetoxy-DMT, wherein the quetiapine is administered to post-treat at least 360 minutes post to the 4-Acetoxy-DMT. In some preferred embodiments, the serotonin receptor modulator is quetiapine and the psychedelic is 4-Acetoxy-DMT, wherein quetiapine is administered to post-treat between about 60 minutes and about 180 minutes post to the administration of 4-Acetoxy-DMT.

In some embodiments, the serotonin receptor modulator is risperidone and the psychedelic is 4-Acetoxy-DMT, wherein the risperidone is administered to post-treat at least 15 minutes post to the administration of 4-Acetoxy-DMT. In some embodiments, the serotonin receptor modulator is risperidone and the psychedelic is 4-Acetoxy-DMT, wherein the risperidone is administered to post-treat at least 30 minutes post to the 4-Acetoxy-DMT. In some embodiments, the serotonin receptor modulator is risperidone and the psychedelic is 4-Acetoxy-DMT, wherein the risperidone is administered to post-treat at least 60 minutes post to the 4-Acetoxy-DMT. In some embodiments, the serotonin receptor modulator is risperidone and the psychedelic is 4-Acetoxy-DMT, wherein the risperidone is administered to post-treat at least 90 minutes post to the 4-Acetoxy-DMT. In some embodiments, the serotonin receptor modulator is risperidone and the psychedelic is 4-Acetoxy-DMT, wherein the risperidone is administered to post-treat at least 120 minutes post to the 4-Acetoxy-DMT. In some embodiments, the serotonin receptor modulator is risperidone and the psychedelic is 4-Acetoxy-DMT, wherein the risperidone is administered to post-treat at least 150 minutes post to the 4-Acetoxy-DMT. In some embodiments, the serotonin receptor modulator is risperidone and the psychedelic is 4-Acetoxy-DMT, wherein the risperidone is administered to post-treat between about 15 minutes and about 150 minutes post to the 4-Acetoxy-DMT. In some embodiments, the serotonin receptor modulator is risperidone and the psychedelic is 4-Acetoxy-DMT, wherein the risperidone is administered to post-treat at least 180 minutes post to the 4-Acetoxy-DMT. In some embodiments, the serotonin receptor modulator is risperidone and the psychedelic is 4-Acetoxy-DMT, wherein the risperidone is administered to post-treat at least 210 minutes post to the 4-Acetoxy-DMT. In some embodiments, the serotonin receptor modulator is risperidone and the psychedelic is 4-Acetoxy-DMT, wherein the risperidone is administered to post-treat at least 240 minutes post to the 4-Acetoxy-DMT. In some embodiments, the serotonin receptor modulator is risperidone and the psychedelic is 4-Acetoxy-DMT, wherein the risperidone is administered to post-treat at least 270 minutes post to the 4-Acetoxy-DMT. In some embodiments, the serotonin receptor modulator is risperidone and the psychedelic is 4-Acetoxy-DMT, wherein the risperidone is administered to post-treat at least 300 minutes post to the 4-Acetoxy-DMT. In some embodiments, the serotonin receptor modulator is risperidone and the psychedelic is 4-Acetoxy-DMT, wherein the risperidone is administered to post-treat at least 330 minutes post to the 4-Acetoxy-DMT. In some embodiments, the serotonin receptor modulator is risperidone and the psychedelic is 4-Acetoxy-DMT, wherein the risperidone is administered to post-treat at least 360 minutes post to the 4-Acetoxy-DMT. In some preferred embodiments, the serotonin receptor modulator is risperidone and the psychedelic is 4-Acetoxy-DMT, wherein risperidone is administered to post-treat between about 60 minutes and about 180 minutes post to the administration of 4-Acetoxy-DMT.

In some embodiments, the serotonin receptor modulator is eplivanserin and the psychedelic is LSD, wherein the eplivanserin is administered to post-treat at least 15 minutes post to the administration of LSD. In some embodiments, the serotonin receptor modulator is eplivanserin and the psychedelic is LSD, wherein the eplivanserin is administered to post-treat at least 30 minutes post to the LSD. In some embodiments, the serotonin receptor modulator is eplivanserin and the psychedelic is LSD, wherein the eplivanserin is administered to post-treat at least 60 minutes post to the LSD. In some embodiments, the serotonin receptor modulator is eplivanserin and the psychedelic is LSD, wherein the eplivanserin is administered to post-treat at least 90 minutes post to the LSD. In some embodiments, the serotonin receptor modulator is eplivanserin and the psychedelic is LSD, wherein the eplivanserin is administered to post-treat at least 120 minutes post to the LSD. In some embodiments, the serotonin receptor modulator is eplivanserin and the psychedelic is LSD, wherein the eplivanserin is administered to post-treat at least 150 minutes post to the LSD. In some embodiments, the serotonin receptor modulator is eplivanserin and the psychedelic is LSD, wherein the eplivanserin is administered to post-treat between about 15 minutes and about 150 minutes post to the LSD. In some embodiments, the serotonin receptor modulator is eplivanserin and the psychedelic is LSD, wherein the eplivanserin is administered to post-treat at least 180 minutes post to the LSD. In some embodiments, the serotonin receptor modulator is eplivanserin and the psychedelic is LSD, wherein the eplivanserin is administered to post-treat at least 210 minutes post to the LSD. In some embodiments, the serotonin receptor modulator is eplivanserin and the psychedelic is LSD, wherein the eplivanserin is administered to post-treat at least 240 minutes post to the LSD. In some embodiments, the serotonin receptor modulator is eplivanserin and the psychedelic is LSD, wherein the eplivanserin is administered to post-treat at least 270 minutes post to the LSD. In some embodiments, the serotonin receptor modulator is eplivanserin and the psychedelic is LSD, wherein the eplivanserin is administered to post-treat at least 300 minutes post to the LSD. In some embodiments, the serotonin receptor modulator is eplivanserin and the psychedelic is LSD, wherein the eplivanserin is administered to post-treat at least 330 minutes post to the LSD. In some embodiments, the serotonin receptor modulator is eplivanserin and the psychedelic is LSD, wherein the eplivanserin is administered to post-treat at least 360 minutes post to the LSD.

In some preferred embodiments, the serotonin receptor modulator is eplivanserin and the psychedelic is LSD, wherein eplivanserin is administered to post-treat between about 60 minutes and about 180 minutes post to the administration of LSD.

In some embodiments, the serotonin receptor modulator is volinanserin and the psychedelic is LSD, wherein the volinanserin is administered to post-treat at least 15 minutes post to the administration of LSD. In some embodiments, the serotonin receptor modulator is volinanserin and the psychedelic is LSD, wherein the volinanserin is administered to post-treat at least 30 minutes post to the LSD. In some embodiments, the serotonin receptor modulator is volinanserin and the psychedelic is LSD, wherein the volinanserin is administered to post-treat at least 60 minutes post to the LSD. In some embodiments, the serotonin receptor modulator is volinanserin and the psychedelic is LSD, wherein the volinanserin is administered to post-treat at least 90 minutes post to the LSD. In some embodiments, the serotonin receptor modulator is volinanserin and the psychedelic is LSD, wherein the volinanserin is administered to post-treat at least 120 minutes post to the LSD. In some embodiments, the serotonin receptor modulator is volinanserin and the psychedelic is LSD, wherein the volinanserin is administered to post-treat at least 150 minutes post to the LSD. In some embodiments, the serotonin receptor modulator is volinanserin and the psychedelic is LSD, wherein the volinanserin is administered to post-treat between about 15 minutes and about 150 minutes post to the LSD. In some embodiments, the serotonin receptor modulator is volinanserin and the psychedelic is LSD, wherein the volinanserin is administered to post-treat at least 180 minutes post to the LSD. In some embodiments, the serotonin receptor modulator is volinanserin and the psychedelic is LSD, wherein the volinanserin is administered to post-treat at least 210 minutes post to the LSD. In some embodiments, the serotonin receptor modulator is volinanserin and the psychedelic is LSD, wherein the volinanserin is administered to post-treat at least 240 minutes post to the LSD. In some embodiments, the serotonin receptor modulator is volinanserin and the psychedelic is LSD, wherein the volinanserin is administered to post-treat at least 270 minutes post to the LSD. In some embodiments, the serotonin receptor modulator is volinanserin and the psychedelic is LSD, wherein the volinanserin is administered to post-treat at least 300 minutes post to the LSD. In some embodiments, the serotonin receptor modulator is volinanserin and the psychedelic is LSD, wherein the volinanserin is administered to post-treat at least 330 minutes post to the LSD. In some embodiments, the serotonin receptor modulator is volinanserin and the psychedelic is LSD, wherein the volinanserin is administered to post-treat at least 360 minutes post to the LSD. In some preferred embodiments, the serotonin receptor modulator is volinanserin and the psychedelic is LSD, wherein volinanserin is administered to post-treat between about 60 minutes and about 180 minutes post to the administration of LSD.

In some embodiments, the serotonin receptor modulator is ketanserin and the psychedelic is LSD, wherein the ketanserin is administered to post-treat at least 15 minutes post to the administration of LSD. In some embodiments, the serotonin receptor modulator is ketanserin and the psychedelic is LSD, wherein the ketanserin is administered to post-treat at least 30 minutes post to the LSD. In some embodiments, the serotonin receptor modulator is ketanserin and the psychedelic is LSD, wherein the ketanserin is administered to post-treat at least 60 minutes post to the LSD. In some embodiments, the serotonin receptor modulator is ketanserin and the psychedelic is LSD, wherein the ketanserin is administered to post-treat at least 90 minutes post to the LSD. In some embodiments, the serotonin receptor modulator is ketanserin and the psychedelic is LSD, wherein the ketanserin is administered to post-treat at least 120 minutes post to the LSD. In some embodiments, the serotonin receptor modulator is ketanserin and the psychedelic is LSD, wherein the ketanserin is administered to post-treat at least 150 minutes post to the LSD. In some embodiments, the serotonin receptor modulator is ketanserin and the psychedelic is LSD, wherein the ketanserin is administered to post-treat between about 15 minutes and about 150 minutes post to the LSD. In some embodiments, the serotonin receptor modulator is ketanserin and the psychedelic is LSD, wherein the ketanserin is administered to post-treat at least 180 minutes post to the LSD. In some embodiments, the serotonin receptor modulator is ketanserin and the psychedelic is LSD, wherein the ketanserin is administered to post-treat at least 210 minutes post to the LSD. In some embodiments, the serotonin receptor modulator is ketanserin and the psychedelic is LSD, wherein the ketanserin is administered to post-treat at least 240 minutes post to the LSD. In some embodiments, the serotonin receptor modulator is ketanserin and the psychedelic is LSD, wherein the ketanserin is administered to post-treat at least 270 minutes post to the LSD. In some embodiments, the serotonin receptor modulator is ketanserin and the psychedelic is LSD, wherein the ketanserin is administered to post-treat at least 300 minutes post to the LSD. In some embodiments, the serotonin receptor modulator is ketanserin and the psychedelic is LSD, wherein the ketanserin is administered to post-treat at least 330 minutes post to the LSD. In some embodiments, the serotonin receptor modulator is ketanserin and the psychedelic is LSD, wherein the ketanserin is administered to post-treat at least 360 minutes post to the LSD. In some preferred embodiments, the serotonin receptor modulator is ketanserin and the psychedelic is LSD, wherein ketanserin is administered to post-treat between about 60 minutes and about 180 minutes post to the administration of LSD.

In some embodiments, the serotonin receptor modulator is ritanserin and the psychedelic is LSD, wherein the ritanserin is administered to post-treat at least 15 minutes post to the administration of LSD. In some embodiments, the serotonin receptor modulator is ritanserin and the psychedelic is LSD, wherein the ritanserin is administered to post-treat at least 30 minutes post to the LSD. In some embodiments, the serotonin receptor modulator is ritanserin and the psychedelic is LSD, wherein the ritanserin is administered to post-treat at least 60 minutes post to the LSD. In some embodiments, the serotonin receptor modulator is ritanserin and the psychedelic is LSD, wherein the ritanserin is administered to post-treat at least 90 minutes post to the LSD. In some embodiments, the serotonin receptor modulator is ritanserin and the psychedelic is LSD, wherein the ritanserin is administered to post-treat at least 120 minutes post to the LSD. In some embodiments, the serotonin receptor modulator is ritanserin and the psychedelic is LSD, wherein the ritanserin is administered to post-treat at least 150 minutes post to the LSD. In some embodiments, the serotonin receptor modulator is ritanserin and the psychedelic is LSD, wherein the ritanserin is administered to post-treat between about 15 minutes and about 150 minutes post to the LSD. In some embodiments, the serotonin receptor modulator is ritanserin and the psychedelic is LSD, wherein the ritanserin is administered to post-treat at least 180 minutes post to the LSD. In some embodiments, the serotonin receptor modulator is ritanserin and the psychedelic is LSD, wherein the ritanserin is administered to post-treat at least 210 minutes post to the LSD. In some embodiments, the serotonin receptor modulator is ritanserin and the psychedelic is LSD, wherein the ritanserin is administered to post-treat at least 240 minutes post to the LSD. In some embodiments, the serotonin receptor modulator is ritanserin and the psychedelic is LSD, wherein the ritanserin is administered to post-treat at least 270 minutes post to the LSD. In some embodiments, the serotonin receptor modulator is ritanserin and the psychedelic is LSD, wherein the ritanserin is administered to post-treat at least 300 minutes post to the LSD. In some embodiments, the serotonin receptor modulator is ritanserin and the psychedelic is LSD, wherein the ritanserin is administered to post-treat at least 330 minutes post to the LSD. In some embodiments, the serotonin receptor modulator is ritanserin and the psychedelic is LSD, wherein the ritanserin is administered to post-treat at least 360 minutes post to the LSD. In some preferred embodiments, the serotonin receptor modulator is ritanserin and the psychedelic is LSD, wherein ritanserin is administered to post-treat between about 60 minutes and about 180 minutes post to the administration of LSD.

In some embodiments, the serotonin receptor modulator is pimavanserin and the psychedelic is LSD, wherein the pimavanserin is administered to post-treat at least 15 minutes post to the administration of LSD. In some embodiments, the serotonin receptor modulator is pimavanserin and the psychedelic is LSD, wherein the pimavanserin is administered to post-treat at least 30 minutes post to the LSD. In some embodiments, the serotonin receptor modulator is pimavanserin and the psychedelic is LSD, wherein the pimavanserin is administered to post-treat at least 60 minutes post to the LSD. In some embodiments, the serotonin receptor modulator is pimavanserin and the psychedelic is LSD, wherein the pimavanserin is administered to post-treat at least 90 minutes post to the LSD. In some embodiments, the serotonin receptor modulator is pimavanserin and the psychedelic is LSD, wherein the pimavanserin is administered to post-treat at least 120 minutes post to the LSD. In some embodiments, the serotonin receptor modulator is pimavanserin and the psychedelic is LSD, wherein the pimavanserin is administered to post-treat at least 150 minutes post to the LSD. In some embodiments, the serotonin receptor modulator is pimavanserin and the psychedelic is LSD, wherein the pimavanserin is administered to post-treat between about 15 minutes and about 150 minutes post to the LSD. In some embodiments, the serotonin receptor modulator is pimavanserin and the psychedelic is LSD, wherein the pimavanserin is administered to post-treat at least 180 minutes post to the LSD. In some embodiments, the serotonin receptor modulator is pimavanserin and the psychedelic is LSD, wherein the pimavanserin is administered to post-treat at least 210 minutes post to the LSD. In some embodiments, the serotonin receptor modulator is pimavanserin and the psychedelic is LSD, wherein the pimavanserin is administered to post-treat at least 240 minutes post to the LSD. In some embodiments, the serotonin receptor modulator is pimavanserin and the psychedelic is LSD, wherein the pimavanserin is administered to post-treat at least 270 minutes post to the LSD. In some embodiments, the serotonin receptor modulator is pimavanserin and the psychedelic is LSD, wherein the pimavanserin is administered to post-treat at least 300 minutes post to the LSD. In some embodiments, the serotonin receptor modulator is pimavanserin and the psychedelic is LSD, wherein the pimavanserin is administered to post-treat at least 330 minutes post to the LSD. In some embodiments, the serotonin receptor modulator is pimavanserin and the psychedelic is LSD, wherein the pimavanserin is administered to post-treat at least 360 minutes post to the LSD. In some preferred embodiments, the serotonin receptor modulator is pimavanserin and the psychedelic is LSD, wherein pimavanserin is administered to post-treat between about 60 minutes and about 180 minutes post to the administration of LSD.

In some embodiments, the serotonin receptor modulator is nelotanserin and the psychedelic is LSD, wherein the nelotanserin is administered to post-treat at least 15 minutes post to the administration of LSD. In some embodiments, the serotonin receptor modulator is nelotanserin and the psychedelic is LSD, wherein the nelotanserin is administered to post-treat at least 30 minutes post to the LSD. In some embodiments, the serotonin receptor modulator is nelotanserin and the psychedelic is LSD, wherein the nelotanserin is administered to post-treat at least 60 minutes post to the LSD. In some embodiments, the serotonin receptor modulator is nelotanserin and the psychedelic is LSD, wherein the nelotanserin is administered to post-treat at least 90 minutes post to the LSD. In some embodiments, the serotonin receptor modulator is nelotanserin and the psychedelic is LSD, wherein the nelotanserin is administered to post-treat at least 120 minutes post to the LSD. In some embodiments, the serotonin receptor modulator is nelotanserin and the psychedelic is LSD, wherein the nelotanserin is administered to post-treat at least 150 minutes post to the LSD. In some embodiments, the serotonin receptor modulator is nelotanserin and the psychedelic is LSD, wherein the nelotanserin is administered to post-treat between about 15 minutes and about 150 minutes post to the LSD. In some embodiments, the serotonin receptor modulator is nelotanserin and the psychedelic is LSD, wherein the nelotanserin is administered to post-treat at least 180 minutes post to the LSD. In some embodiments, the serotonin receptor modulator is nelotanserin and the psychedelic is LSD, wherein the nelotanserin is administered to post-treat at least 210 minutes post to the LSD. In some embodiments, the serotonin receptor modulator is nelotanserin and the psychedelic is LSD, wherein the nelotanserin is administered to post-treat at least 240 minutes post to the LSD. In some embodiments, the serotonin receptor modulator is nelotanserin and the psychedelic is LSD, wherein the nelotanserin is administered to post-treat at least 270 minutes post to the LSD. In some embodiments, the serotonin receptor modulator is nelotanserin and the psychedelic is LSD, wherein the nelotanserin is administered to post-treat at least 300 minutes post to the LSD. In some embodiments, the serotonin receptor modulator is nelotanserin and the psychedelic is LSD, wherein the nelotanserin is administered to post-treat at least 330 minutes post to the LSD. In some embodiments, the serotonin receptor modulator is nelotanserin and the psychedelic is LSD, wherein the nelotanserin is administered to post-treat at least 360 minutes post to the LSD. In some preferred embodiments, the serotonin receptor modulator is nelotanserin and the psychedelic is LSD, wherein nelotanserin is administered to post-treat between about 60 minutes and about 180 minutes post to the administration of LSD.

In some embodiments, the serotonin receptor modulator is pruvanserin and the psychedelic is LSD, wherein the pruvanserin is administered to post-treat at least 15 minutes post to the administration of LSD. In some embodiments, the serotonin receptor modulator is pruvanserin and the psychedelic is LSD, wherein the pruvanserin is administered to post-treat at least 30 minutes post to the LSD. In some embodiments, the serotonin receptor modulator is pruvanserin and the psychedelic is LSD, wherein the pruvanserin is administered to post-treat at least 60 minutes post to the LSD. In some embodiments, the serotonin receptor modulator is pruvanserin and the psychedelic is LSD, wherein the pruvanserin is administered to post-treat at least 90 minutes post to the LSD. In some embodiments, the serotonin receptor modulator is pruvanserin and the psychedelic is LSD, wherein the pruvanserin is administered to post-treat at least 120 minutes post to the LSD. In some embodiments, the serotonin receptor modulator is pruvanserin and the psychedelic is LSD, wherein the pruvanserin is administered to post-treat at least 150 minutes post to the LSD. In some embodiments, the serotonin receptor modulator is pruvanserin and the psychedelic is LSD, wherein the pruvanserin is administered to post-treat between about 15 minutes and about 150 minutes post to the LSD. In some embodiments, the serotonin receptor modulator is pruvanserin and the psychedelic is LSD, wherein the pruvanserin is administered to post-treat at least 180 minutes post to the LSD. In some embodiments, the serotonin receptor modulator is pruvanserin and the psychedelic is LSD, wherein the pruvanserin is administered to post-treat at least 210 minutes post to the LSD. In some embodiments, the serotonin receptor modulator is pruvanserin and the psychedelic is LSD, wherein the pruvanserin is administered to post-treat at least 240 minutes post to the LSD. In some embodiments, the serotonin receptor modulator is pruvanserin and the psychedelic is LSD, wherein the pruvanserin is administered to post-treat at least 270 minutes post to the LSD. In some embodiments, the serotonin receptor modulator is pruvanserin and the psychedelic is LSD, wherein the pruvanserin is administered to post-treat at least 300 minutes post to the LSD. In some embodiments, the serotonin receptor modulator is pruvanserin and the psychedelic is LSD, wherein the pruvanserin is administered to post-treat at least 330 minutes post to the LSD. In some embodiments, the serotonin receptor modulator is pruvanserin and the psychedelic is LSD, wherein the pruvanserin is administered to post-treat at least 360 minutes post to the LSD. In some preferred embodiments, the serotonin receptor modulator is pruvanserin and the psychedelic is LSD, wherein pruvanserin is administered to post-treat between about 60 minutes and about 180 minutes post to the administration of LSD.

In some embodiments, the serotonin receptor modulator is flibanserin and the psychedelic is LSD, wherein the flibanserin is administered to post-treat at least 15 minutes post to the administration of LSD. In some embodiments, the serotonin receptor modulator is flibanserin and the psychedelic is LSD, wherein the flibanserin is administered to post-treat at least 30 minutes post to the LSD. In some embodiments, the serotonin receptor modulator is flibanserin and the psychedelic is LSD, wherein the flibanserin is administered to post-treat at least 60 minutes post to the LSD. In some embodiments, the serotonin receptor modulator is flibanserin and the psychedelic is LSD, wherein the flibanserin is administered to post-treat at least 90 minutes post to the LSD. In some embodiments, the serotonin receptor modulator is flibanserin and the psychedelic is LSD, wherein the flibanserin is administered to post-treat at least 120 minutes post to the LSD. In some embodiments, the serotonin receptor modulator is flibanserin and the psychedelic is LSD, wherein the flibanserin is administered to post-treat at least 150 minutes post to the LSD. In some embodiments, the serotonin receptor modulator is flibanserin and the psychedelic is LSD, wherein the flibanserin is administered to post-treat between about 15 minutes and about 150 minutes post to the LSD. In some embodiments, the serotonin receptor modulator is flibanserin and the psychedelic is LSD, wherein the flibanserin is administered to post-treat at least 180 minutes post to the LSD. In some embodiments, the serotonin receptor modulator is flibanserin and the psychedelic is LSD, wherein the flibanserin is administered to post-treat at least 210 minutes post to the LSD. In some embodiments, the serotonin receptor modulator is flibanserin and the psychedelic is LSD, wherein the flibanserin is administered to post-treat at least 240 minutes post to the LSD. In some embodiments, the serotonin receptor modulator is flibanserin and the psychedelic is LSD, wherein the flibanserin is administered to post-treat at least 270 minutes post to the LSD. In some embodiments, the serotonin receptor modulator is flibanserin and the psychedelic is LSD, wherein the flibanserin is administered to post-treat at least 300 minutes post to the LSD. In some embodiments, the serotonin receptor modulator is flibanserin and the psychedelic is LSD, wherein the flibanserin is administered to post-treat at least 330 minutes post to the LSD. In some embodiments, the serotonin receptor modulator is flibanserin and the psychedelic is LSD, wherein the flibanserin is administered to post-treat at least 360 minutes post to the LSD. In some preferred embodiments, the serotonin receptor modulator is flibanserin and the psychedelic is LSD, wherein flibanserin is administered to post-treat between about 60 minutes and about 180 minutes post to the administration of LSD.

In some embodiments, the serotonin receptor modulator is olanzapine and the psychedelic is LSD, wherein the olanzapine is administered to post-treat at least 15 minutes post to the administration of LSD. In some embodiments, the serotonin receptor modulator is olanzapine and the psychedelic is LSD, wherein the olanzapine is administered to post-treat at least 30 minutes post to the LSD. In some embodiments, the serotonin receptor modulator is olanzapine and the psychedelic is LSD, wherein the olanzapine is administered to post-treat at least 60 minutes post to the LSD. In some embodiments, the serotonin receptor modulator is olanzapine and the psychedelic is LSD, wherein the olanzapine is administered to post-treat at least 90 minutes post to the LSD. In some embodiments, the serotonin receptor modulator is olanzapine and the psychedelic is LSD, wherein the olanzapine is administered to post-treat at least 120 minutes post to the LSD. In some embodiments, the serotonin receptor modulator is olanzapine and the psychedelic is LSD, wherein the olanzapine is administered to post-treat at least 150 minutes post to the LSD. In some embodiments, the serotonin receptor modulator is olanzapine and the psychedelic is LSD, wherein the olanzapine is administered to post-treat between about 15 minutes and about 150 minutes post to the LSD. In some embodiments, the serotonin receptor modulator is olanzapine and the psychedelic is LSD, wherein the olanzapine is administered to post-treat at least 180 minutes post to the LSD. In some embodiments, the serotonin receptor modulator is olanzapine and the psychedelic is LSD, wherein the olanzapine is administered to post-treat at least 210 minutes post to the LSD. In some embodiments, the serotonin receptor modulator is olanzapine and the psychedelic is LSD, wherein the olanzapine is administered to post-treat at least 240 minutes post to the LSD. In some embodiments, the serotonin receptor modulator is olanzapine and the psychedelic is LSD, wherein the olanzapine is administered to post-treat at least 270 minutes post to the LSD. In some embodiments, the serotonin receptor modulator is olanzapine and the psychedelic is LSD, wherein the olanzapine is administered to post-treat at least 300 minutes post to the LSD. In some embodiments, the serotonin receptor modulator is olanzapine and the psychedelic is LSD, wherein the olanzapine is administered to post-treat at least 330 minutes post to the LSD. In some embodiments, the serotonin receptor modulator is olanzapine and the psychedelic is LSD, wherein the olanzapine is administered to post-treat at least 360 minutes post to the LSD. In some preferred embodiments, the serotonin receptor modulator is olanzapine and the psychedelic is LSD, wherein olanzapine is administered to post-treat between about 60 minutes and about 180 minutes post to the administration of LSD.

In some embodiments, the serotonin receptor modulator is quetiapine and the psychedelic is LSD, wherein the quetiapine is administered to post-treat at least 15 minutes post to the administration of LSD. In some embodiments, the serotonin receptor modulator is quetiapine and the psychedelic is LSD, wherein the quetiapine is administered to post-treat at least 30 minutes post to the LSD. In some embodiments, the serotonin receptor modulator is quetiapine and the psychedelic is LSD, wherein the quetiapine is administered to post-treat at least 60 minutes post to the LSD. In some embodiments, the serotonin receptor modulator is quetiapine and the psychedelic is LSD, wherein the quetiapine is administered to post-treat at least 90 minutes post to the LSD. In some embodiments, the serotonin receptor modulator is quetiapine and the psychedelic is LSD, wherein the quetiapine is administered to post-treat at least 120 minutes post to the LSD. In some embodiments, the serotonin receptor modulator is quetiapine and the psychedelic is LSD, wherein the quetiapine is administered to post-treat at least 150 minutes post to the LSD. In some embodiments, the serotonin receptor modulator is quetiapine and the psychedelic is LSD, wherein the quetiapine is administered to post-treat between about 15 minutes and about 150 minutes post to the LSD. In some embodiments, the serotonin receptor modulator is quetiapine and the psychedelic is LSD, wherein the quetiapine is administered to post-treat at least 180 minutes post to the LSD. In some embodiments, the serotonin receptor modulator is quetiapine and the psychedelic is LSD, wherein the quetiapine is administered to post-treat at least 210 minutes post to the LSD. In some embodiments, the serotonin receptor modulator is quetiapine and the psychedelic is LSD, wherein the quetiapine is administered to post-treat at least 240 minutes post to the LSD. In some embodiments, the serotonin receptor modulator is quetiapine and the psychedelic is LSD, wherein the quetiapine is administered to post-treat at least 270 minutes post to the LSD. In some embodiments, the serotonin receptor modulator is quetiapine and the psychedelic is LSD, wherein the quetiapine is administered to post-treat at least 300 minutes post to the LSD. In some embodiments, the serotonin receptor modulator is quetiapine and the psychedelic is LSD, wherein the quetiapine is administered to post-treat at least 330 minutes post to the LSD. In some embodiments, the serotonin receptor modulator is quetiapine and the psychedelic is LSD, wherein the quetiapine is administered to post-treat at least 360 minutes post to the LSD. In some preferred embodiments, the serotonin receptor modulator is quetiapine and the psychedelic is LSD, wherein quetiapine is administered to post-treat between about 60 minutes and about 180 minutes post to the administration of LSD.

In some embodiments, the serotonin receptor modulator is risperidone and the psychedelic is LSD, wherein the risperidone is administered to post-treat at least 15 minutes post to the administration of LSD. In some embodiments, the serotonin receptor modulator is risperidone and the psychedelic is LSD, wherein the risperidone is administered to post-treat at least 30 minutes post to the LSD. In some embodiments, the serotonin receptor modulator is risperidone and the psychedelic is LSD, wherein the risperidone is administered to post-treat at least 60 minutes post to the LSD. In some embodiments, the serotonin receptor modulator is risperidone and the psychedelic is LSD, wherein the risperidone is administered to post-treat at least 90 minutes post to the LSD. In some embodiments, the serotonin receptor modulator is risperidone and the psychedelic is LSD, wherein the risperidone is administered to post-treat at least 120 minutes post to the LSD. In some embodiments, the serotonin receptor modulator is risperidone and the psychedelic is LSD, wherein the risperidone is administered to post-treat at least 150 minutes post to the LSD. In some embodiments, the serotonin receptor modulator is risperidone and the psychedelic is LSD, wherein the risperidone is administered to post-treat between about 15 minutes and about 150 minutes post to the LSD. In some embodiments, the serotonin receptor modulator is risperidone and the psychedelic is LSD, wherein the risperidone is administered to post-treat at least 180 minutes post to the LSD. In some embodiments, the serotonin receptor modulator is risperidone and the psychedelic is LSD, wherein the risperidone is administered to post-treat at least 210 minutes post to the LSD. In some embodiments, the serotonin receptor modulator is risperidone and the psychedelic is LSD, wherein the risperidone is administered to post-treat at least 240 minutes post to the LSD. In some embodiments, the serotonin receptor modulator is risperidone and the psychedelic is LSD, wherein the risperidone is administered to post-treat at least 270 minutes post to the LSD. In some embodiments, the serotonin receptor modulator is risperidone and the psychedelic is LSD, wherein the risperidone is administered to post-treat at least 300 minutes post to the LSD. In some embodiments, the serotonin receptor modulator is risperidone and the psychedelic is LSD, wherein the risperidone is administered to post-treat at least 330 minutes post to the LSD. In some embodiments, the serotonin receptor modulator is risperidone and the psychedelic is LSD, wherein the risperidone is administered to post-treat at least 360 minutes post to the LSD. In some preferred embodiments, the serotonin receptor modulator is risperidone and the psychedelic is LSD, wherein risperidone is administered to post-treat between about 60 minutes and about 180 minutes post to the administration of LSD.

In some embodiments, the serotonin receptor modulator is eplivanserin and the psychedelic is ALD-52, wherein the eplivanserin is administered to post-treat at least 15 minutes post to the administration of ALD-52. In some embodiments, the serotonin receptor modulator is eplivanserin and the psychedelic is ALD-52, wherein the eplivanserin is administered to post-treat at least 30 minutes post to the ALD-52. In some embodiments, the serotonin receptor modulator is eplivanserin and the psychedelic is ALD-52, wherein the eplivanserin is administered to post-treat at least 60 minutes post to the ALD-52. In some embodiments, the serotonin receptor modulator is eplivanserin and the psychedelic is ALD-52, wherein the eplivanserin is administered to post-treat at least 90 minutes post to the ALD-52. In some embodiments, the serotonin receptor modulator is eplivanserin and the psychedelic is ALD-52, wherein the eplivanserin is administered to post-treat at least 120 minutes post to the ALD-52. In some embodiments, the serotonin receptor modulator is eplivanserin and the psychedelic is ALD-52, wherein the eplivanserin is administered to post-treat at least 150 minutes post to the ALD-52. In some embodiments, the serotonin receptor modulator is eplivanserin and the psychedelic is ALD-52, wherein the eplivanserin is administered to post-treat between about 15 minutes and about 150 minutes post to the ALD-52. In some embodiments, the serotonin receptor modulator is eplivanserin and the psychedelic is ALD-52, wherein the eplivanserin is administered to post-treat at least 180 minutes post to the ALD-52. In some embodiments, the serotonin receptor modulator is eplivanserin and the psychedelic is ALD-52, wherein the eplivanserin is administered to post-treat at least 210 minutes post to the ALD-52. In some embodiments, the serotonin receptor modulator is eplivanserin and the psychedelic is ALD-52, wherein the eplivanserin is administered to post-treat at least 240 minutes post to the ALD-52. In some embodiments, the serotonin receptor modulator is eplivanserin and the psychedelic is ALD-52, wherein the eplivanserin is administered to post-treat at least 270 minutes post to the ALD-52. In some embodiments, the serotonin receptor modulator is eplivanserin and the psychedelic is ALD-52, wherein the eplivanserin is administered to post-treat at least 300 minutes post to the ALD-52. In some embodiments, the serotonin receptor modulator is eplivanserin and the psychedelic is ALD-52, wherein the eplivanserin is administered to post-treat at least 330 minutes post to the ALD-52. In some embodiments, the serotonin receptor modulator is eplivanserin and the psychedelic is ALD-52, wherein the eplivanserin is administered to post-treat at least 360 minutes post to the ALD-52.

In some preferred embodiments, the serotonin receptor modulator is eplivanserin and the psychedelic is ALD-52, wherein eplivanserin is administered to post-treat between about 60 minutes and about 180 minutes post to the administration of ALD-52.

In some embodiments, the serotonin receptor modulator is volinanserin and the psychedelic is ALD-52, wherein the volinanserin is administered to post-treat at least 15 minutes post to the ALD-52. In some embodiments, the serotonin receptor modulator is volinanserin and the psychedelic is ALD-52, wherein the volinanserin is administered to post-treat at least 30 minutes post to the ALD-52. In some embodiments, the serotonin receptor modulator is volinanserin and the psychedelic is ALD-52, wherein the volinanserin is administered to post-treat at least 60 minutes post to the ALD-52. In some embodiments, the serotonin receptor modulator is volinanserin and the psychedelic is ALD-52, wherein the volinanserin is administered to post-treat at least 90 minutes post to the ALD-52. In some embodiments, the serotonin receptor modulator is volinanserin and the psychedelic is ALD-52, wherein the volinanserin is administered to post-treat at least 120 minutes post to the ALD-52. In some embodiments, the serotonin receptor modulator is volinanserin and the psychedelic is ALD-52, wherein the volinanserin is administered to post-treat at least 150 minutes post to the ALD-52. In some embodiments, the serotonin receptor modulator is volinanserin and the psychedelic is ALD-52, wherein the volinanserin is administered to post-treat between about 15 minutes and about 150 minutes post to the ALD-52. In some embodiments, the serotonin receptor modulator is volinanserin and the psychedelic is ALD-52, wherein the volinanserin is administered to post-treat at least 180 minutes post to the ALD-52. In some embodiments, the serotonin receptor modulator is volinanserin and the psychedelic is ALD-52, wherein the volinanserin is administered to post-treat at least 210 minutes post to the ALD-52. In some embodiments, the serotonin receptor modulator is volinanserin and the psychedelic is ALD-52, wherein the volinanserin is administered to post-treat at least 240 minutes post to the ALD-52. In some embodiments, the serotonin receptor modulator is volinanserin and the psychedelic is ALD-52, wherein the volinanserin is administered to post-treat at least 270 minutes post to the ALD-52. In some embodiments, the serotonin receptor modulator is volinanserin and the psychedelic is ALD-52, wherein the volinanserin is administered to post-treat at least 300 minutes post to the ALD-52. In some embodiments, the serotonin receptor modulator is volinanserin and the psychedelic is ALD-52, wherein the volinanserin is administered to post-treat at least 330 minutes post to the ALD-52. In some embodiments, the serotonin receptor modulator is volinanserin and the psychedelic is ALD-52, wherein the volinanserin is administered to post-treat at least 360 minutes post to the ALD-52. In some preferred embodiments, the serotonin receptor modulator is volinanserin and the psychedelic is ALD-52, wherein volinanserin is administered to post-treat between about 60 minutes and about 180 minutes post to the administration of ALD-52.

In some embodiments, the serotonin receptor modulator is ketanserin and the psychedelic is ALD-52, wherein the ketanserin is administered to post-treat at least 15 minutes post to the administration of ALD-52. In some embodiments, the serotonin receptor modulator is ketanserin and the psychedelic is ALD-52, wherein the ketanserin is administered to post-treat at least 30 minutes post to the ALD-52. In some embodiments, the serotonin receptor modulator is ketanserin and the psychedelic is ALD-52, wherein the ketanserin is administered to post-treat at least 60 minutes post to the ALD-52. In some embodiments, the serotonin receptor modulator is ketanserin and the psychedelic is ALD-52, wherein the ketanserin is administered to post-treat at least 90 minutes post to the ALD-52. In some embodiments, the serotonin receptor modulator is ketanserin and the psychedelic is ALD-52, wherein the ketanserin is administered to post-treat at least 120 minutes post to the ALD-52. In some embodiments, the serotonin receptor modulator is ketanserin and the psychedelic is ALD-52, wherein the ketanserin is administered to post-treat at least 150 minutes post to the ALD-52. In some embodiments, the serotonin receptor modulator is ketanserin and the psychedelic is ALD-52, wherein the ketanserin is administered to post-treat between about 15 minutes and about 150 minutes post to the ALD-52. In some embodiments, the serotonin receptor modulator is ketanserin and the psychedelic is ALD-52, wherein the ketanserin is administered to post-treat at least 180 minutes post to the ALD-52. In some embodiments, the serotonin receptor modulator is ketanserin and the psychedelic is ALD-52, wherein the ketanserin is administered to post-treat at least 210 minutes post to the ALD-52. In some embodiments, the serotonin receptor modulator is ketanserin and the psychedelic is ALD-52, wherein the ketanserin is administered to post-treat at least 240 minutes post to the ALD-52. In some embodiments, the serotonin receptor modulator is ketanserin and the psychedelic is ALD-52, wherein the ketanserin is administered to post-treat at least 270 minutes post to the ALD-52. In some embodiments, the serotonin receptor modulator is ketanserin and the psychedelic is ALD-52, wherein the ketanserin is administered to post-treat at least 300 minutes post to the ALD-52. In some embodiments, the serotonin receptor modulator is ketanserin and the psychedelic is ALD-52, wherein the ketanserin is administered to post-treat at least 330 minutes post to the ALD-52. In some embodiments, the serotonin receptor modulator is ketanserin and the psychedelic is ALD-52, wherein the ketanserin is administered to post-treat at least 360 minutes post to the ALD-52. In some preferred embodiments, the serotonin receptor modulator is ketanserin and the psychedelic is ALD-52, wherein ketanserin is administered to post-treat between about 60 minutes and about 180 minutes post to the administration of ALD-52.

In some embodiments, the serotonin receptor modulator is ritanserin and the psychedelic is ALD-52, wherein the ritanserin is administered to post-treat at least 15 minutes post to the ALD-52. In some embodiments, the serotonin receptor modulator is ritanserin and the psychedelic is ALD-52, wherein the ritanserin is administered to post-treat at least 30 minutes post to the ALD-52. In some embodiments, the serotonin receptor modulator is ritanserin and the psychedelic is ALD-52, wherein the ritanserin is administered to post-treat at least 60 minutes post to the ALD-52. In some embodiments, the serotonin receptor modulator is ritanserin and the psychedelic is ALD-52, wherein the ritanserin is administered to post-treat at least 90 minutes post to the ALD-52. In some embodiments, the serotonin receptor modulator is ritanserin and the psychedelic is ALD-52, wherein the ritanserin is administered to post-treat at least 120 minutes post to the ALD-52. In some embodiments, the serotonin receptor modulator is ritanserin and the psychedelic is ALD-52, wherein the ritanserin is administered to post-treat at least 150 minutes post to the ALD-52. In some embodiments, the serotonin receptor modulator is ritanserin and the psychedelic is ALD-52, wherein the ritanserin is administered to post-treat between about 15 minutes and about 150 minutes post to the ALD-52. In some embodiments, the serotonin receptor modulator is ritanserin and the psychedelic is ALD-52, wherein the ritanserin is administered to post-treat at least 180 minutes post to the ALD-52. In some embodiments, the serotonin receptor modulator is ritanserin and the psychedelic is ALD-52, wherein the ritanserin is administered to post-treat at least 210 minutes post to the ALD-52. In some embodiments, the serotonin receptor modulator is ritanserin and the psychedelic is ALD-52, wherein the ritanserin is administered to post-treat at least 240 minutes post to the ALD-52. In some embodiments, the serotonin receptor modulator is ritanserin and the psychedelic is ALD-52, wherein the ritanserin is administered to post-treat at least 270 minutes post to the ALD-52. In some embodiments, the serotonin receptor modulator is ritanserin and the psychedelic is ALD-52, wherein the ritanserin is administered to post-treat at least 300 minutes post to the ALD-52. In some embodiments, the serotonin receptor modulator is ritanserin and the psychedelic is ALD-52, wherein the ritanserin is administered to post-treat at least 330 minutes post to the ALD-52. In some embodiments, the serotonin receptor modulator is ritanserin and the psychedelic is ALD-52, wherein the ritanserin is administered to post-treat at least 360 minutes post to the ALD-52. In some preferred embodiments, the serotonin receptor modulator is ritanserin and the psychedelic is ALD-52, wherein ritanserin is administered to post-treat between about 60 minutes and about 180 minutes post to the administration of ALD-52.

In some embodiments, the serotonin receptor modulator is pimavanserin and the psychedelic is ALD-52, wherein the pimavanserin is administered to post-treat at least 15 minutes post to the ALD-52. In some embodiments, the serotonin receptor modulator is pimavanserin and the psychedelic is ALD-52, wherein the pimavanserin is administered to post-treat at least 30 minutes post to the ALD-52. In some embodiments, the serotonin receptor modulator is pimavanserin and the psychedelic is ALD-52, wherein the pimavanserin is administered to post-treat at least 60 minutes post to the ALD-52. In some embodiments, the serotonin receptor modulator is pimavanserin and the psychedelic is ALD-52, wherein the pimavanserin is administered to post-treat at least 90 minutes post to the ALD-52. In some embodiments, the serotonin receptor modulator is pimavanserin and the psychedelic is ALD-52, wherein the pimavanserin is administered to post-treat at least 120 minutes post to the ALD-52. In some embodiments, the serotonin receptor modulator is pimavanserin and the psychedelic is ALD-52, wherein the pimavanserin is administered to post-treat at least 150 minutes post to the ALD-52. In some embodiments, the serotonin receptor modulator is pimavanserin and the psychedelic is ALD-52, wherein the pimavanserin is administered to post-treat between about 15 minutes and about 150 minutes post to the ALD-52. In some embodiments, the serotonin receptor modulator is pimavanserin and the psychedelic is ALD-52, wherein the pimavanserin is administered to post-treat at least 180 minutes post to the ALD-52. In some embodiments, the serotonin receptor modulator is pimavanserin and the psychedelic is ALD-52, wherein the pimavanserin is administered to post-treat at least 210 minutes post to the ALD-52. In some embodiments, the serotonin receptor modulator is pimavanserin and the psychedelic is ALD-52, wherein the pimavanserin is administered to post-treat at least 240 minutes post to the ALD-52. In some embodiments, the serotonin receptor modulator is pimavanserin and the psychedelic is ALD-52, wherein the pimavanserin is administered to post-treat at least 270 minutes post to the ALD-52. In some embodiments, the serotonin receptor modulator is pimavanserin and the psychedelic is ALD-52, wherein the pimavanserin is administered to post-treat at least 300 minutes post to the ALD-52. In some embodiments, the serotonin receptor modulator is pimavanserin and the psychedelic is ALD-52, wherein the pimavanserin is administered to post-treat at least 330 minutes post to the ALD-52. In some embodiments, the serotonin receptor modulator is pimavanserin and the psychedelic is ALD-52, wherein the pimavanserin is administered to post-treat at least 360 minutes post to the ALD-52. In some preferred embodiments, the serotonin receptor modulator is pimavanserin and the psychedelic is ALD-52, wherein pimavanserin is administered to post-treat between about 60 minutes and about 180 minutes post to the administration of ALD-52.

In some embodiments, the serotonin receptor modulator is nelotanserin and the psychedelic is ALD-52, wherein the nelotanserin is administered to post-treat at least 15 minutes post to the ALD-52. In some embodiments, the serotonin receptor modulator is nelotanserin and the psychedelic is ALD-52, wherein the nelotanserin is administered to post-treat at least 30 minutes post to the ALD-52. In some embodiments, the serotonin receptor modulator is nelotanserin and the psychedelic is ALD-52, wherein the nelotanserin is administered to post-treat at least 60 minutes post to the ALD-52. In some embodiments, the serotonin receptor modulator is nelotanserin and the psychedelic is ALD-52, wherein the nelotanserin is administered to post-treat at least 90 minutes post to the ALD-52. In some embodiments, the serotonin receptor modulator is nelotanserin and the psychedelic is ALD-52, wherein the nelotanserin is administered to post-treat at least 120 minutes post to the ALD-52. In some embodiments, the serotonin receptor modulator is nelotanserin and the psychedelic is ALD-52, wherein the nelotanserin is administered to post-treat at least 150 minutes post to the ALD-52. In some embodiments, the serotonin receptor modulator is nelotanserin and the psychedelic is ALD-52, wherein the nelotanserin is administered to post-treat between about 15 minutes and about 150 minutes post to the ALD-52. In some embodiments, the serotonin receptor modulator is nelotanserin and the psychedelic is ALD-52, wherein the nelotanserin is administered to post-treat at least 180 minutes post to the ALD-52. In some embodiments, the serotonin receptor modulator is nelotanserin and the psychedelic is ALD-52, wherein the nelotanserin is administered to post-treat at least 210 minutes post to the ALD-52. In some embodiments, the serotonin receptor modulator is nelotanserin and the psychedelic is ALD-52, wherein the nelotanserin is administered to post-treat at least 240 minutes post to the ALD-52. In some embodiments, the serotonin receptor modulator is nelotanserin and the psychedelic is ALD-52, wherein the nelotanserin is administered to post-treat at least 270 minutes post to the ALD-52. In some embodiments, the serotonin receptor modulator is nelotanserin and the psychedelic is ALD-52, wherein the nelotanserin is administered to post-treat at least 300 minutes post to the ALD-52. In some embodiments, the serotonin receptor modulator is nelotanserin and the psychedelic is ALD-52, wherein the nelotanserin is administered to post-treat at least 330 minutes post to the ALD-52. In some embodiments, the serotonin receptor modulator is nelotanserin and the psychedelic is ALD-52, wherein the nelotanserin is administered to post-treat at least 360 minutes post to the ALD-52. In some preferred embodiments, the serotonin receptor modulator is nelotanserin and the psychedelic is ALD-52, wherein nelotanserin is administered to post-treat between about 60 minutes and about 180 minutes post to the administration of ALD-52.

In some embodiments, the serotonin receptor modulator is pruvanserin and the psychedelic is ALD-52, wherein the pruvanserin is administered to post-treat at least 15 minutes post to the ALD-52. In some embodiments, the serotonin receptor modulator is pruvanserin and the psychedelic is ALD-52, wherein the pruvanserin is administered to post-treat at least 30 minutes post to the ALD-52. In some embodiments, the serotonin receptor modulator is pruvanserin and the psychedelic is ALD-52, wherein the pruvanserin is administered to post-treat at least 60 minutes post to the ALD-52. In some embodiments, the serotonin receptor modulator is pruvanserin and the psychedelic is ALD-52, wherein the pruvanserin is administered to post-treat at least 90 minutes post to the ALD-52. In some embodiments, the serotonin receptor modulator is pruvanserin and the psychedelic is ALD-52, wherein the pruvanserin is administered to post-treat at least 120 minutes post to the ALD-52. In some embodiments, the serotonin receptor modulator is pruvanserin and the psychedelic is ALD-52, wherein the pruvanserin is administered to post-treat at least 150 minutes post to the ALD-52. In some embodiments, the serotonin receptor modulator is pruvanserin and the psychedelic is ALD-52, wherein the pruvanserin is administered to post-treat between about 15 minutes and about 150 minutes post to the ALD-52. In some embodiments, the serotonin receptor modulator is pruvanserin and the psychedelic is ALD-52, wherein the pruvanserin is administered to post-treat at least 180 minutes post to the ALD-52. In some embodiments, the serotonin receptor modulator is pruvanserin and the psychedelic is ALD-52, wherein the pruvanserin is administered to post-treat at least 210 minutes post to the ALD-52. In some embodiments, the serotonin receptor modulator is pruvanserin and the psychedelic is ALD-52, wherein the pruvanserin is administered to post-treat at least 240 minutes post to the ALD-52. In some embodiments, the serotonin receptor modulator is pruvanserin and the psychedelic is ALD-52, wherein the pruvanserin is administered to post-treat at least 270 minutes post to the ALD-52. In some embodiments, the serotonin receptor modulator is pruvanserin and the psychedelic is ALD-52, wherein the pruvanserin is administered to post-treat at least 300 minutes post to the ALD-52. In some embodiments, the serotonin receptor modulator is pruvanserin and the psychedelic is ALD-52, wherein the pruvanserin is administered to post-treat at least 330 minutes post to the ALD-52. In some embodiments, the serotonin receptor modulator is pruvanserin and the psychedelic is ALD-52, wherein the pruvanserin is administered to post-treat at least 360 minutes post to the ALD-52. In some preferred embodiments, the serotonin receptor modulator is pruvanserin and the psychedelic is ALD-52, wherein pruvanserin is administered to post-treat between about 60 minutes and about 180 minutes post to the administration of ALD-52.

In some embodiments, the serotonin receptor modulator is flibanserin and the psychedelic is ALD-52, wherein the flibanserin is administered to post-treat at least 15 minutes post to the administration of ALD-52. In some embodiments, the serotonin receptor modulator is flibanserin and the psychedelic is ALD-52, wherein the flibanserin is administered to post-treat at least 30 minutes post to the ALD-52. In some embodiments, the serotonin receptor modulator is flibanserin and the psychedelic is ALD-52, wherein the flibanserin is administered to post-treat at least 60 minutes post to the ALD-52. In some embodiments, the serotonin receptor modulator is flibanserin and the psychedelic is ALD-52, wherein the flibanserin is administered to post-treat at least 90 minutes post to the ALD-52. In some embodiments, the serotonin receptor modulator is flibanserin and the psychedelic is ALD-52, wherein the flibanserin is administered to post-treat at least 120 minutes post to the ALD-52. In some embodiments, the serotonin receptor modulator is flibanserin and the psychedelic is ALD-52, wherein the flibanserin is administered to post-treat at least 150 minutes post to the ALD-52. In some embodiments, the serotonin receptor modulator is flibanserin and the psychedelic is ALD-52, wherein the flibanserin is administered to post-treat between about 15 minutes and about 150 minutes post to the ALD-52. In some embodiments, the serotonin receptor modulator is flibanserin and the psychedelic is ALD-52, wherein the flibanserin is administered to post-treat at least 180 minutes post to the ALD-52. In some embodiments, the serotonin receptor modulator is flibanserin and the psychedelic is ALD-52, wherein the flibanserin is administered to post-treat at least 210 minutes post to the ALD-52. In some embodiments, the serotonin receptor modulator is flibanserin and the psychedelic is ALD-52, wherein the flibanserin is administered to post-treat at least 240 minutes post to the ALD-52. In some embodiments, the serotonin receptor modulator is flibanserin and the psychedelic is ALD-52, wherein the flibanserin is administered to post-treat at least 270 minutes post to the ALD-52. In some embodiments, the serotonin receptor modulator is flibanserin and the psychedelic is ALD-52, wherein the flibanserin is administered to post-treat at least 300 minutes post to the ALD-52. In some embodiments, the serotonin receptor modulator is flibanserin and the psychedelic is ALD-52, wherein the flibanserin is administered to post-treat at least 330 minutes post to the ALD-52. In some embodiments, the serotonin receptor modulator is flibanserin and the psychedelic is ALD-52, wherein the flibanserin is administered to post-treat at least 360 minutes post to the ALD-52. In some preferred embodiments, the serotonin receptor modulator is flibanserin and the psychedelic is ALD-52, wherein flibanserin is administered to post-treat between about 60 minutes and about 180 minutes post to the administration of ALD-52.

In some embodiments, the serotonin receptor modulator is olanzapine and the psychedelic is ALD-52, wherein the olanzapine is administered to post-treat at least 15 minutes post to the administration of ALD-52. In some embodiments, the serotonin receptor modulator is olanzapine and the psychedelic is ALD-52, wherein the olanzapine is administered to post-treat at least 30 minutes post to the ALD-52. In some embodiments, the serotonin receptor modulator is olanzapine and the psychedelic is ALD-52, wherein the olanzapine is administered to post-treat at least 60 minutes post to the ALD-52. In some embodiments, the serotonin receptor modulator is olanzapine and the psychedelic is ALD-52, wherein the olanzapine is administered to post-treat at least 90 minutes post to the ALD-52. In some embodiments, the serotonin receptor modulator is olanzapine and the psychedelic is ALD-52, wherein the olanzapine is administered to post-treat at least 120 minutes post to the ALD-52. In some embodiments, the serotonin receptor modulator is olanzapine and the psychedelic is ALD-52, wherein the olanzapine is administered to post-treat at least 150 minutes post to the ALD-52. In some embodiments, the serotonin receptor modulator is olanzapine and the psychedelic is ALD-52, wherein the olanzapine is administered to post-treat between about 15 minutes and about 150 minutes post to the ALD-52. In some embodiments, the serotonin receptor modulator is olanzapine and the psychedelic is ALD-52, wherein the olanzapine is administered to post-treat at least 180 minutes post to the ALD-52. In some embodiments, the serotonin receptor modulator is olanzapine and the psychedelic is ALD-52, wherein the olanzapine is administered to post-treat at least 210 minutes post to the ALD-52. In some embodiments, the serotonin receptor modulator is olanzapine and the psychedelic is ALD-52, wherein the olanzapine is administered to post-treat at least 240 minutes post to the ALD-52. In some embodiments, the serotonin receptor modulator is olanzapine and the psychedelic is ALD-52, wherein the olanzapine is administered to post-treat at least 270 minutes post to the ALD-52. In some embodiments, the serotonin receptor modulator is olanzapine and the psychedelic is ALD-52, wherein the olanzapine is administered to post-treat at least 300 minutes post to the ALD-52. In some embodiments, the serotonin receptor modulator is olanzapine and the psychedelic is ALD-52, wherein the olanzapine is administered to post-treat at least 330 minutes post to the ALD-52. In some embodiments, the serotonin receptor modulator is olanzapine and the psychedelic is ALD-52, wherein the olanzapine is administered to post-treat at least 360 minutes post to the ALD-52. In some preferred embodiments, the serotonin receptor modulator is olanzapine and the psychedelic is ALD-52, wherein olanzapine is administered to post-treat between about 60 minutes and about 180 minutes post to the administration of ALD-52.

In some embodiments, the serotonin receptor modulator is quetiapine and the psychedelic is ALD-52, wherein the quetiapine is administered to post-treat at least 15 minutes post to the administration of ALD-52. In some embodiments, the serotonin receptor modulator is quetiapine and the psychedelic is ALD-52, wherein the quetiapine is administered to post-treat at least 30 minutes post to the ALD-52. In some embodiments, the serotonin receptor modulator is quetiapine and the psychedelic is ALD-52, wherein the quetiapine is administered to post-treat at least 60 minutes post to the ALD-52. In some embodiments, the serotonin receptor modulator is quetiapine and the psychedelic is ALD-52, wherein the quetiapine is administered to post-treat at least 90 minutes post to the ALD-52. In some embodiments, the serotonin receptor modulator is quetiapine and the psychedelic is ALD-52, wherein the quetiapine is administered to post-treat at least 120 minutes post to the ALD-52. In some embodiments, the serotonin receptor modulator is quetiapine and the psychedelic is ALD-52, wherein the quetiapine is administered to post-treat at least 150 minutes post to the ALD-52. In some embodiments, the serotonin receptor modulator is quetiapine and the psychedelic is ALD-52, wherein the quetiapine is administered to post-treat between about 15 minutes and about 150 minutes post to the ALD-52. In some embodiments, the serotonin receptor modulator is quetiapine and the psychedelic is ALD-52, wherein the quetiapine is administered to post-treat at least 180 minutes post to the ALD-52. In some embodiments, the serotonin receptor modulator is quetiapine and the psychedelic is ALD-52, wherein the quetiapine is administered to post-treat at least 210 minutes post to the ALD-52. In some embodiments, the serotonin receptor modulator is quetiapine and the psychedelic is ALD-52, wherein the quetiapine is administered to post-treat at least 240 minutes post to the ALD-52. In some embodiments, the serotonin receptor modulator is quetiapine and the psychedelic is ALD-52, wherein the quetiapine is administered to post-treat at least 270 minutes post to the ALD-52. In some embodiments, the serotonin receptor modulator is quetiapine and the psychedelic is ALD-52, wherein the quetiapine is administered to post-treat at least 300 minutes post to the ALD-52. In some embodiments, the serotonin receptor modulator is quetiapine and the psychedelic is ALD-52, wherein the quetiapine is administered to post-treat at least 330 minutes post to the ALD-52. In some embodiments, the serotonin receptor modulator is quetiapine and the psychedelic is ALD-52, wherein the quetiapine is administered to post-treat at least 360 minutes post to the ALD-52. In some preferred embodiments, the serotonin receptor modulator is quetiapine and the psychedelic is ALD-52, wherein quetiapine is administered to post-treat between about 60 minutes and about 180 minutes post to the administration of ALD-52.

In some embodiments, the serotonin receptor modulator is risperidone and the psychedelic is ALD-52, wherein the risperidone is administered to post-treat at least 15 minutes post to the administration of ALD-52. In some embodiments, the serotonin receptor modulator is risperidone and the psychedelic is ALD-52, wherein the risperidone is administered to post-treat at least 30 minutes post to the ALD-52. In some embodiments, the serotonin receptor modulator is risperidone and the psychedelic is ALD-52, wherein the risperidone is administered to post-treat at least 60 minutes post to the ALD-52. In some embodiments, the serotonin receptor modulator is risperidone and the psychedelic is ALD-52, wherein the risperidone is administered to post-treat at least 90 minutes post to the ALD-52. In some embodiments, the serotonin receptor modulator is risperidone and the psychedelic is ALD-52, wherein the risperidone is administered to post-treat at least 120 minutes post to the ALD-52. In some embodiments, the serotonin receptor modulator is risperidone and the psychedelic is ALD-52, wherein the risperidone is administered to post-treat at least 150 minutes post to the ALD-52. In some embodiments, the serotonin receptor modulator is risperidone and the psychedelic is ALD-52, wherein the risperidone is administered to post-treat between about 15 minutes and about 150 minutes post to the ALD-52. In some embodiments, the serotonin receptor modulator is risperidone and the psychedelic is ALD-52, wherein the risperidone is administered to post-treat at least 180 minutes post to the ALD-52. In some embodiments, the serotonin receptor modulator is risperidone and the psychedelic is ALD-52, wherein the risperidone is administered to post-treat at least 210 minutes post to the ALD-52. In some embodiments, the serotonin receptor modulator is risperidone and the psychedelic is ALD-52, wherein the risperidone is administered to post-treat at least 240 minutes post to the ALD-52. In some embodiments, the serotonin receptor modulator is risperidone and the psychedelic is ALD-52, wherein the risperidone is administered to post-treat at least 270 minutes post to the ALD-52. In some embodiments, the serotonin receptor modulator is risperidone and the psychedelic is ALD-52, wherein the risperidone is administered to post-treat at least 300 minutes post to the ALD-52. In some embodiments, the serotonin receptor modulator is risperidone and the psychedelic is ALD-52, wherein the risperidone is administered to post-treat at least 330 minutes post to the ALD-52. In some embodiments, the serotonin receptor modulator is risperidone and the psychedelic is ALD-52, wherein the risperidone is administered to post-treat at least 360 minutes post to the ALD-52. In some preferred embodiments, the serotonin receptor modulator is risperidone and the psychedelic is ALD-52, wherein risperidone is administered to post-treat between about 60 minutes and about 180 minutes post to the administration of ALD-52.

In some embodiments, the serotonin receptor modulator is eplivanserin and the psychedelic is 1P-LSD, wherein the eplivanserin is administered to post-treat at least 15 minutes post to the administration of 1P-LSD. In some embodiments, the serotonin receptor modulator is eplivanserin and the psychedelic is 1P-LSD, wherein the eplivanserin is administered to post-treat at least 30 minutes post to the 1P-LSD. In some embodiments, the serotonin receptor modulator is eplivanserin and the psychedelic is 1P-LSD, wherein the eplivanserin is administered to post-treat at least 60 minutes post to the 1P-LSD. In some embodiments, the serotonin receptor modulator is eplivanserin and the psychedelic is 1P-LSD, wherein the eplivanserin is administered to post-treat at least 90 minutes post to the 1P-LSD. In some embodiments, the serotonin receptor modulator is eplivanserin and the psychedelic is 1P-LSD, wherein the eplivanserin is administered to post-treat at least 120 minutes post to the 1P-LSD. In some embodiments, the serotonin receptor modulator is eplivanserin and the psychedelic is 1P-LSD, wherein the eplivanserin is administered to post-treat at least 150 minutes post to the 1P-LSD. In some embodiments, the serotonin receptor modulator is eplivanserin and the psychedelic is 1P-LSD, wherein the eplivanserin is administered to post-treat between about 15 minutes and about 150 minutes post to the 1P-LSD. In some embodiments, the serotonin receptor modulator is eplivanserin and the psychedelic is 1P-LSD, wherein the eplivanserin is administered to post-treat at least 180 minutes post to the 1P-LSD. In some embodiments, the serotonin receptor modulator is eplivanserin and the psychedelic is 1P-LSD, wherein the eplivanserin is administered to post-treat at least 210 minutes post to the 1P-LSD. In some embodiments, the serotonin receptor modulator is eplivanserin and the psychedelic is 1P-LSD, wherein the eplivanserin is administered to post-treat at least 240 minutes post to the 1P-LSD. In some embodiments, the serotonin receptor modulator is eplivanserin and the psychedelic is 1P-LSD, wherein the eplivanserin is administered to post-treat at least 270 minutes post to the 1P-LSD. In some embodiments, the serotonin receptor modulator is eplivanserin and the psychedelic is 1P-LSD, wherein the eplivanserin is administered to post-treat at least 300 minutes post to the 1P-LSD. In some embodiments, the serotonin receptor modulator is eplivanserin and the psychedelic is 1P-LSD, wherein the eplivanserin is administered to post-treat at least 330 minutes post to the 1P-LSD. In some embodiments, the serotonin receptor modulator is eplivanserin and the psychedelic is 1P-LSD, wherein the eplivanserin is administered to post-treat at least 360 minutes post to the 1P-LSD.

In some preferred embodiments, the serotonin receptor modulator is eplivanserin and the psychedelic is 1P-LSD, wherein eplivanserin is administered to post-treat between about 60 minutes and about 180 minutes post to the administration of 1P-LSD.

In some embodiments, the serotonin receptor modulator is volinanserin and the psychedelic is 1P-LSD, wherein the volinanserin is administered to post-treat at least 15 minutes post to the 1P-LSD. In some embodiments, the serotonin receptor modulator is volinanserin and the psychedelic is 1P-LSD, wherein the volinanserin is administered to post-treat at least 30 minutes post to the 1P-LSD. In some embodiments, the serotonin receptor modulator is volinanserin and the psychedelic is 1P-LSD, wherein the volinanserin is administered to post-treat at least 60 minutes post to the 1P-LSD. In some embodiments, the serotonin receptor modulator is volinanserin and the psychedelic is 1P-LSD, wherein the volinanserin is administered to post-treat at least 90 minutes post to the 1P-LSD. In some embodiments, the serotonin receptor modulator is volinanserin and the psychedelic is 1P-LSD, wherein the volinanserin is administered to post-treat at least 120 minutes post to the 1P-LSD. In some embodiments, the serotonin receptor modulator is volinanserin and the psychedelic is 1P-LSD, wherein the volinanserin is administered to post-treat at least 150 minutes post to the 1P-LSD. In some embodiments, the serotonin receptor modulator is volinanserin and the psychedelic is 1P-LSD, wherein the volinanserin is administered to post-treat between about 15 minutes and about 150 minutes post to the 1P-LSD. In some embodiments, the serotonin receptor modulator is volinanserin and the psychedelic is 1P-LSD, wherein the volinanserin is administered to post-treat at least 180 minutes post to the 1P-LSD. In some embodiments, the serotonin receptor modulator is volinanserin and the psychedelic is 1P-LSD, wherein the volinanserin is administered to post-treat at least 210 minutes post to the 1P-LSD. In some embodiments, the serotonin receptor modulator is volinanserin and the psychedelic is 1P-LSD, wherein the volinanserin is administered to post-treat at least 240 minutes post to the 1P-LSD. In some embodiments, the serotonin receptor modulator is volinanserin and the psychedelic is 1P-LSD, wherein the volinanserin is administered to post-treat at least 270 minutes post to the 1P-LSD. In some embodiments, the serotonin receptor modulator is volinanserin and the psychedelic is 1P-LSD, wherein the volinanserin is administered to post-treat at least 300 minutes post to the 1P-LSD. In some embodiments, the serotonin receptor modulator is volinanserin and the psychedelic is 1P-LSD, wherein the volinanserin is administered to post-treat at least 330 minutes post to the 1P-LSD. In some embodiments, the serotonin receptor modulator is volinanserin and the psychedelic is 1P-LSD, wherein the volinanserin is administered to post-treat at least 360 minutes post to the 1P-LSD. In some preferred embodiments, the serotonin receptor modulator is volinanserin and the psychedelic is 1P-LSD, wherein volinanserin is administered to post-treat between about 60 minutes and about 180 minutes post to the administration of 1P-LSD.

In some embodiments, the serotonin receptor modulator is ketanserin and the psychedelic is 1P-LSD, wherein the ketanserin is administered to post-treat at least 15 minutes post to the administration of 1P-LSD. In some embodiments, the serotonin receptor modulator is ketanserin and the psychedelic is 1P-LSD, wherein the ketanserin is administered to post-treat at least 30 minutes post to the 1P-LSD. In some embodiments, the serotonin receptor modulator is ketanserin and the psychedelic is 1P-LSD, wherein the ketanserin is administered to post-treat at least 60 minutes post to the 1P-LSD. In some embodiments, the serotonin receptor modulator is ketanserin and the psychedelic is 1P-LSD, wherein the ketanserin is administered to post-treat at least 90 minutes post to the 1P-LSD. In some embodiments, the serotonin receptor modulator is ketanserin and the psychedelic is 1P-LSD, wherein the ketanserin is administered to post-treat at least 120 minutes post to the 1P-LSD. In some embodiments, the serotonin receptor modulator is ketanserin and the psychedelic is 1P-LSD, wherein the ketanserin is administered to post-treat at least 150 minutes post to the 1P-LSD. In some embodiments, the serotonin receptor modulator is ketanserin and the psychedelic is 1P-LSD, wherein the ketanserin is administered to post-treat between about 15 minutes and about 150 minutes post to the 1P-LSD. In some embodiments, the serotonin receptor modulator is ketanserin and the psychedelic is 1P-LSD, wherein the ketanserin is administered to post-treat at least 180 minutes post to the 1P-LSD. In some embodiments, the serotonin receptor modulator is ketanserin and the psychedelic is 1P-LSD, wherein the ketanserin is administered to post-treat at least 210 minutes post to the 1P-LSD. In some embodiments, the serotonin receptor modulator is ketanserin and the psychedelic is 1P-LSD, wherein the ketanserin is administered to post-treat at least 240 minutes post to the 1P-LSD. In some embodiments, the serotonin receptor modulator is ketanserin and the psychedelic is 1P-LSD, wherein the ketanserin is administered to post-treat at least 270 minutes post to the 1P-LSD. In some embodiments, the serotonin receptor modulator is ketanserin and the psychedelic is 1P-LSD, wherein the ketanserin is administered to post-treat at least 300 minutes post to the 1P-LSD. In some embodiments, the serotonin receptor modulator is ketanserin and the psychedelic is 1P-LSD, wherein the ketanserin is administered to post-treat at least 330 minutes post to the 1P-LSD. In some embodiments, the serotonin receptor modulator is ketanserin and the psychedelic is 1P-LSD, wherein the ketanserin is administered to post-treat at least 360 minutes post to the 1P-LSD. In some preferred embodiments, the serotonin receptor modulator is ketanserin and the psychedelic is 1P-LSD, wherein ketanserin is administered to post-treat between about 60 minutes and about 180 minutes post to the administration of 1P-LSD.

In some embodiments, the serotonin receptor modulator is ritanserin and the psychedelic is 1P-LSD, wherein the ritanserin is administered to post-treat at least 15 minutes post to the 1P-LSD. In some embodiments, the serotonin receptor modulator is ritanserin and the psychedelic is 1P-LSD, wherein the ritanserin is administered to post-treat at least 30 minutes post to the 1P-LSD. In some embodiments, the serotonin receptor modulator is ritanserin and the psychedelic is 1P-LSD, wherein the ritanserin is administered to post-treat at least 60 minutes post to the 1P-LSD. In some embodiments, the serotonin receptor modulator is ritanserin and the psychedelic is 1P-LSD, wherein the ritanserin is administered to post-treat at least 90 minutes post to the 1P-LSD. In some embodiments, the serotonin receptor modulator is ritanserin and the psychedelic is 1P-LSD, wherein the ritanserin is administered to post-treat at least 120 minutes post to the 1P-LSD. In some embodiments, the serotonin receptor modulator is ritanserin and the psychedelic is 1P-LSD, wherein the ritanserin is administered to post-treat at least 150 minutes post to the 1P-LSD. In some embodiments, the serotonin receptor modulator is ritanserin and the psychedelic is 1P-LSD, wherein the ritanserin is administered to post-treat between about 15 minutes and about 150 minutes post to the 1P-LSD. In some embodiments, the serotonin receptor modulator is ritanserin and the psychedelic is 1P-LSD, wherein the ritanserin is administered to post-treat at least 180 minutes post to the 1P-LSD. In some embodiments, the serotonin receptor modulator is ritanserin and the psychedelic is 1P-LSD, wherein the ritanserin is administered to post-treat at least 210 minutes post to the 1P-LSD. In some embodiments, the serotonin receptor modulator is ritanserin and the psychedelic is 1P-LSD, wherein the ritanserin is administered to post-treat at least 240 minutes post to the 1P-LSD. In some embodiments, the serotonin receptor modulator is ritanserin and the psychedelic is 1P-LSD, wherein the ritanserin is administered to post-treat at least 270 minutes post to the 1P-LSD. In some embodiments, the serotonin receptor modulator is ritanserin and the psychedelic is 1P-LSD, wherein the ritanserin is administered to post-treat at least 300 minutes post to the 1P-LSD. In some embodiments, the serotonin receptor modulator is ritanserin and the psychedelic is 1P-LSD, wherein the ritanserin is administered to post-treat at least 330 minutes post to the 1P-LSD. In some embodiments, the serotonin receptor modulator is ritanserin and the psychedelic is 1P-LSD, wherein the ritanserin is administered to post-treat at least 360 minutes post to the 1P-LSD. In some preferred embodiments, the serotonin receptor modulator is ritanserin and the psychedelic is 1P-LSD, wherein ritanserin is administered to post-treat between about 60 minutes and about 180 minutes post to the administration of 1P-LSD.

In some embodiments, the serotonin receptor modulator is pimavanserin and the psychedelic is 1P-LSD, wherein the pimavanserin is administered to post-treat at least 15 minutes post to the 1P-LSD. In some embodiments, the serotonin receptor modulator is pimavanserin and the psychedelic is 1P-LSD, wherein the pimavanserin is administered to post-treat at least 30 minutes post to the 1P-LSD. In some embodiments, the serotonin receptor modulator is pimavanserin and the psychedelic is 1P-LSD, wherein the pimavanserin is administered to post-treat at least 60 minutes post to the 1P-LSD. In some embodiments, the serotonin receptor modulator is pimavanserin and the psychedelic is 1P-LSD, wherein the pimavanserin is administered to post-treat at least 90 minutes post to the 1P-LSD. In some embodiments, the serotonin receptor modulator is pimavanserin and the psychedelic is 1P-LSD, wherein the pimavanserin is administered to post-treat at least 120 minutes post to the 1P-LSD. In some embodiments, the serotonin receptor modulator is pimavanserin and the psychedelic is 1P-LSD, wherein the pimavanserin is administered to post-treat at least 150 minutes post to the 1P-LSD. In some embodiments, the serotonin receptor modulator is pimavanserin and the psychedelic is 1P-LSD, wherein the pimavanserin is administered to post-treat between about 15 minutes and about 150 minutes post to the 1P-LSD. In some embodiments, the serotonin receptor modulator is pimavanserin and the psychedelic is 1P-LSD, wherein the pimavanserin is administered to post-treat at least 180 minutes post to the 1P-LSD. In some embodiments, the serotonin receptor modulator is pimavanserin and the psychedelic is 1P-LSD, wherein the pimavanserin is administered to post-treat at least 210 minutes post to the 1P-LSD. In some embodiments, the serotonin receptor modulator is pimavanserin and the psychedelic is 1P-LSD, wherein the pimavanserin is administered to post-treat at least 240 minutes post to the 1P-LSD. In some embodiments, the serotonin receptor modulator is pimavanserin and the psychedelic is 1P-LSD, wherein the pimavanserin is administered to post-treat at least 270 minutes post to the 1P-LSD. In some embodiments, the serotonin receptor modulator is pimavanserin and the psychedelic is 1P-LSD, wherein the pimavanserin is administered to post-treat at least 300 minutes post to the 1P-LSD. In some embodiments, the serotonin receptor modulator is pimavanserin and the psychedelic is 1P-LSD, wherein the pimavanserin is administered to post-treat at least 330 minutes post to the 1P-LSD. In some embodiments, the serotonin receptor modulator is pimavanserin and the psychedelic is 1P-LSD, wherein the pimavanserin is administered to post-treat at least 360 minutes post to the 1P-LSD. In some preferred embodiments, the serotonin receptor modulator is pimavanserin and the psychedelic is 1P-LSD, wherein pimavanserin is administered to post-treat between about 60 minutes and about 180 minutes post to the administration of 1P-LSD.

In some embodiments, the serotonin receptor modulator is nelotanserin and the psychedelic is 1P-LSD, wherein the nelotanserin is administered to post-treat at least 15 minutes post to the 1P-LSD. In some embodiments, the serotonin receptor modulator is nelotanserin and the psychedelic is 1P-LSD, wherein the nelotanserin is administered to post-treat at least 30 minutes post to the 1P-LSD. In some embodiments, the serotonin receptor modulator is nelotanserin and the psychedelic is 1P-LSD, wherein the nelotanserin is administered to post-treat at least 60 minutes post to the 1P-LSD. In some embodiments, the serotonin receptor modulator is nelotanserin and the psychedelic is 1P-LSD, wherein the nelotanserin is administered to post-treat at least 90 minutes post to the 1P-LSD. In some embodiments, the serotonin receptor modulator is nelotanserin and the psychedelic is 1P-LSD, wherein the nelotanserin is administered to post-treat at least 120 minutes post to the 1P-LSD. In some embodiments, the serotonin receptor modulator is nelotanserin and the psychedelic is 1P-LSD, wherein the nelotanserin is administered to post-treat at least 150 minutes post to the 1P-LSD. In some embodiments, the serotonin receptor modulator is nelotanserin and the psychedelic is 1P-LSD, wherein the nelotanserin is administered to post-treat between about 15 minutes and about 150 minutes post to the 1P-LSD. In some embodiments, the serotonin receptor modulator is nelotanserin and the psychedelic is 1P-LSD, wherein the nelotanserin is administered to post-treat at least 180 minutes post to the 1P-LSD. In some embodiments, the serotonin receptor modulator is nelotanserin and the psychedelic is 1P-LSD, wherein the nelotanserin is administered to post-treat at least 210 minutes post to the 1P-LSD. In some embodiments, the serotonin receptor modulator is nelotanserin and the psychedelic is 1P-LSD, wherein the nelotanserin is administered to post-treat at least 240 minutes post to the 1P-LSD. In some embodiments, the serotonin receptor modulator is nelotanserin and the psychedelic is 1P-LSD, wherein the nelotanserin is administered to post-treat at least 270 minutes post to the 1P-LSD. In some embodiments, the serotonin receptor modulator is nelotanserin and the psychedelic is 1P-LSD, wherein the nelotanserin is administered to post-treat at least 300 minutes post to the 1P-LSD. In some embodiments, the serotonin receptor modulator is nelotanserin and the psychedelic is 1P-LSD, wherein the nelotanserin is administered to post-treat at least 330 minutes post to the 1P-LSD. In some embodiments, the serotonin receptor modulator is nelotanserin and the psychedelic is 1P-LSD, wherein the nelotanserin is administered to post-treat at least 360 minutes post to the 1P-LSD. In some preferred embodiments, the serotonin receptor modulator is nelotanserin and the psychedelic is 1P-LSD, wherein nelotanserin is administered to post-treat between about 60 minutes and about 180 minutes post to the administration of 1P-LSD.

In some embodiments, the serotonin receptor modulator is pruvanserin and the psychedelic is 1P-LSD, wherein the pruvanserin is administered to post-treat at least 15 minutes post to the 1P-LSD. In some embodiments, the serotonin receptor modulator is pruvanserin and the psychedelic is 1P-LSD, wherein the pruvanserin is administered to post-treat at least 30 minutes post to the 1P-LSD. In some embodiments, the serotonin receptor modulator is pruvanserin and the psychedelic is 1P-LSD, wherein the pruvanserin is administered to post-treat at least 60 minutes post to the 1P-LSD. In some embodiments, the serotonin receptor modulator is pruvanserin and the psychedelic is 1P-LSD, wherein the pruvanserin is administered to post-treat at least 90 minutes post to the 1P-LSD. In some embodiments, the serotonin receptor modulator is pruvanserin and the psychedelic is 1P-LSD, wherein the pruvanserin is administered to post-treat at least 120 minutes post to the 1P-LSD. In some embodiments, the serotonin receptor modulator is pruvanserin and the psychedelic is 1P-LSD, wherein the pruvanserin is administered to post-treat at least 150 minutes post to the 1P-LSD. In some embodiments, the serotonin receptor modulator is pruvanserin and the psychedelic is 1P-LSD, wherein the pruvanserin is administered to post-treat between about 15 minutes and about 150 minutes post to the 1P-LSD. In some embodiments, the serotonin receptor modulator is pruvanserin and the psychedelic is 1P-LSD, wherein the pruvanserin is administered to post-treat at least 180 minutes post to the 1P-LSD. In some embodiments, the serotonin receptor modulator is pruvanserin and the psychedelic is 1P-LSD, wherein the pruvanserin is administered to post-treat at least 210 minutes post to the 1P-LSD. In some embodiments, the serotonin receptor modulator is pruvanserin and the psychedelic is 1P-LSD, wherein the pruvanserin is administered to post-treat at least 240 minutes post to the 1P-LSD. In some embodiments, the serotonin receptor modulator is pruvanserin and the psychedelic is 1P-LSD, wherein the pruvanserin is administered to post-treat at least 270 minutes post to the 1P-LSD. In some embodiments, the serotonin receptor modulator is pruvanserin and the psychedelic is 1P-LSD, wherein the pruvanserin is administered to post-treat at least 300 minutes post to the 1P-LSD. In some embodiments, the serotonin receptor modulator is pruvanserin and the psychedelic is 1P-LSD, wherein the pruvanserin is administered to post-treat at least 330 minutes post to the 1P-LSD. In some embodiments, the serotonin receptor modulator is pruvanserin and the psychedelic is 1P-LSD, wherein the pruvanserin is administered to post-treat at least 360 minutes post to the 1P-LSD. In some preferred embodiments, the serotonin receptor modulator is pruvanserin and the psychedelic is 1P-LSD, wherein pruvanserin is administered to post-treat between about 60 minutes and about 180 minutes post to the administration of 1P-LSD.

In some embodiments, the serotonin receptor modulator is flibanserin and the psychedelic is 1P-LSD, wherein the flibanserin is administered to post-treat at least 15 minutes post to the administration of 1P-LSD. In some embodiments, the serotonin receptor modulator is flibanserin and the psychedelic is 1P-LSD, wherein the flibanserin is administered to post-treat at least 30 minutes post to the 1P-LSD. In some embodiments, the serotonin receptor modulator is flibanserin and the psychedelic is 1P-LSD, wherein the flibanserin is administered to post-treat at least 60 minutes post to the 1P-LSD. In some embodiments, the serotonin receptor modulator is flibanserin and the psychedelic is 1P-LSD, wherein the flibanserin is administered to post-treat at least 90 minutes post to the 1P-LSD. In some embodiments, the serotonin receptor modulator is flibanserin and the psychedelic is 1P-LSD, wherein the flibanserin is administered to post-treat at least 120 minutes post to the 1P-LSD. In some embodiments, the serotonin receptor modulator is flibanserin and the psychedelic is 1P-LSD, wherein the flibanserin is administered to post-treat at least 150 minutes post to the 1P-LSD. In some embodiments, the serotonin receptor modulator is flibanserin and the psychedelic is 1P-LSD, wherein the flibanserin is administered to post-treat between about 15 minutes and about 150 minutes post to the 1P-LSD. In some embodiments, the serotonin receptor modulator is flibanserin and the psychedelic is 1P-LSD, wherein the flibanserin is administered to post-treat at least 180 minutes post to the 1P-LSD. In some embodiments, the serotonin receptor modulator is flibanserin and the psychedelic is 1P-LSD, wherein the flibanserin is administered to post-treat at least 210 minutes post to the 1P-LSD. In some embodiments, the serotonin receptor modulator is flibanserin and the psychedelic is 1P-LSD, wherein the flibanserin is administered to post-treat at least 240 minutes post to the 1P-LSD. In some embodiments, the serotonin receptor modulator is flibanserin and the psychedelic is 1P-LSD, wherein the flibanserin is administered to post-treat at least 270 minutes post to the 1P-LSD. In some embodiments, the serotonin receptor modulator is flibanserin and the psychedelic is 1P-LSD, wherein the flibanserin is administered to post-treat at least 300 minutes post to the 1P-LSD. In some embodiments, the serotonin receptor modulator is flibanserin and the psychedelic is 1P-LSD, wherein the flibanserin is administered to post-treat at least 330 minutes post to the 1P-LSD. In some embodiments, the serotonin receptor modulator is flibanserin and the psychedelic is 1P-LSD, wherein the flibanserin is administered to post-treat at least 360 minutes post to the 1P-LSD. In some preferred embodiments, the serotonin receptor modulator is flibanserin and the psychedelic is 1P-LSD, wherein flibanserin is administered to post-treat between about 60 minutes and about 180 minutes post to the administration of 1P-LSD.

In some embodiments, the serotonin receptor modulator is olanzapine and the psychedelic is 1P-LSD, wherein the olanzapine is administered to post-treat at least 15 minutes post to the administration of 1P-LSD. In some embodiments, the serotonin receptor modulator is olanzapine and the psychedelic is 1P-LSD, wherein the olanzapine is administered to post-treat at least 30 minutes post to the 1P-LSD. In some embodiments, the serotonin receptor modulator is olanzapine and the psychedelic is 1P-LSD, wherein the olanzapine is administered to post-treat at least 60 minutes post to the 1P-LSD. In some embodiments, the serotonin receptor modulator is olanzapine and the psychedelic is 1P-LSD, wherein the olanzapine is administered to post-treat at least 90 minutes post to the 1P-LSD. In some embodiments, the serotonin receptor modulator is olanzapine and the psychedelic is 1P-LSD, wherein the olanzapine is administered to post-treat at least 120 minutes post to the 1P-LSD. In some embodiments, the serotonin receptor modulator is olanzapine and the psychedelic is 1P-LSD, wherein the olanzapine is administered to post-treat at least 150 minutes post to the 1P-LSD. In some embodiments, the serotonin receptor modulator is olanzapine and the psychedelic is 1P-LSD, wherein the olanzapine is administered to post-treat between about 15 minutes and about 150 minutes post to the 1P-LSD. In some embodiments, the serotonin receptor modulator is olanzapine and the psychedelic is 1P-LSD, wherein the olanzapine is administered to post-treat at least 180 minutes post to the 1P-LSD. In some embodiments, the serotonin receptor modulator is olanzapine and the psychedelic is 1P-LSD, wherein the olanzapine is administered to post-treat at least 210 minutes post to the 1P-LSD. In some embodiments, the serotonin receptor modulator is olanzapine and the psychedelic is 1P-LSD, wherein the olanzapine is administered to post-treat at least 240 minutes post to the 1P-LSD. In some embodiments, the serotonin receptor modulator is olanzapine and the psychedelic is 1P-LSD, wherein the olanzapine is administered to post-treat at least 270 minutes post to the 1P-LSD. In some embodiments, the serotonin receptor modulator is olanzapine and the psychedelic is 1P-LSD, wherein the olanzapine is administered to post-treat at least 300 minutes post to the 1P-LSD. In some embodiments, the serotonin receptor modulator is olanzapine and the psychedelic is 1P-LSD, wherein the olanzapine is administered to post-treat at least 330 minutes post to the 1P-LSD. In some embodiments, the serotonin receptor modulator is olanzapine and the psychedelic is 1P-LSD, wherein the olanzapine is administered to post-treat at least 360 minutes post to the 1P-LSD. In some preferred embodiments, the serotonin receptor modulator is olanzapine and the psychedelic is 1P-LSD, wherein olanzapine is administered to post-treat between about 60 minutes and about 180 minutes post to the administration of 1P-LSD.

In some embodiments, the serotonin receptor modulator is quetiapine and the psychedelic is 1P-LSD, wherein the quetiapine is administered to post-treat at least 15 minutes post to the administration of 1P-LSD. In some embodiments, the serotonin receptor modulator is quetiapine and the psychedelic is 1P-LSD, wherein the quetiapine is administered to post-treat at least 30 minutes post to the 1P-LSD. In some embodiments, the serotonin receptor modulator is quetiapine and the psychedelic is 1P-LSD, wherein the quetiapine is administered to post-treat at least 60 minutes post to the 1P-LSD. In some embodiments, the serotonin receptor modulator is quetiapine and the psychedelic is 1P-LSD, wherein the quetiapine is administered to post-treat at least 90 minutes post to the 1P-LSD. In some embodiments, the serotonin receptor modulator is quetiapine and the psychedelic is 1P-LSD, wherein the quetiapine is administered to post-treat at least 120 minutes post to the 1P-LSD. In some embodiments, the serotonin receptor modulator is quetiapine and the psychedelic is 1P-LSD, wherein the quetiapine is administered to post-treat at least 150 minutes post to the 1P-LSD. In some embodiments, the serotonin receptor modulator is quetiapine and the psychedelic is 1P-LSD, wherein the quetiapine is administered to post-treat between about 15 minutes and about 150 minutes post to the 1P-LSD. In some embodiments, the serotonin receptor modulator is quetiapine and the psychedelic is 1P-LSD, wherein the quetiapine is administered to post-treat at least 180 minutes post to the 1P-LSD. In some embodiments, the serotonin receptor modulator is quetiapine and the psychedelic is 1P-LSD, wherein the quetiapine is administered to post-treat at least 210 minutes post to the 1P-LSD. In some embodiments, the serotonin receptor modulator is quetiapine and the psychedelic is 1P-LSD, wherein the quetiapine is administered to post-treat at least 240 minutes post to the 1P-LSD. In some embodiments, the serotonin receptor modulator is quetiapine and the psychedelic is 1P-LSD, wherein the quetiapine is administered to post-treat at least 270 minutes post to the 1P-LSD. In some embodiments, the serotonin receptor modulator is quetiapine and the psychedelic is 1P-LSD, wherein the quetiapine is administered to post-treat at least 300 minutes post to the 1P-LSD. In some embodiments, the serotonin receptor modulator is quetiapine and the psychedelic is 1P-LSD, wherein the quetiapine is administered to post-treat at least 330 minutes post to the 1P-LSD. In some embodiments, the serotonin receptor modulator is quetiapine and the psychedelic is 1P-LSD, wherein the quetiapine is administered to post-treat at least 360 minutes post to the 1P-LSD. In some preferred embodiments, the serotonin receptor modulator is quetiapine and the psychedelic is 1P-LSD, wherein quetiapine is administered to post-treat between about 60 minutes and about 180 minutes post to the administration of 1P-LSD.

In some embodiments, the serotonin receptor modulator is risperidone and the psychedelic is 1P-LSD, wherein the risperidone is administered to post-treat at least 15 minutes post to the administration of 1P-LSD. In some embodiments, the serotonin receptor modulator is risperidone and the psychedelic is 1P-LSD, wherein the risperidone is administered to post-treat at least 30 minutes post to the 1P-LSD. In some embodiments, the serotonin receptor modulator is risperidone and the psychedelic is 1P-LSD, wherein the risperidone is administered to post-treat at least 60 minutes post to the 1P-LSD. In some embodiments, the serotonin receptor modulator is risperidone and the psychedelic is 1P-LSD, wherein the risperidone is administered to post-treat at least 90 minutes post to the 1P-LSD. In some embodiments, the serotonin receptor modulator is risperidone and the psychedelic is 1P-LSD, wherein the risperidone is administered to post-treat at least 120 minutes post to the 1P-LSD. In some embodiments, the serotonin receptor modulator is risperidone and the psychedelic is 1P-LSD, wherein the risperidone is administered to post-treat at least 150 minutes post to the 1P-LSD. In some embodiments, the serotonin receptor modulator is risperidone and the psychedelic is 1P-LSD, wherein the risperidone is administered to post-treat between about 15 minutes and about 150 minutes post to the 1P-LSD. In some embodiments, the serotonin receptor modulator is risperidone and the psychedelic is 1P-LSD, wherein the risperidone is administered to post-treat at least 180 minutes post to the 1P-LSD. In some embodiments, the serotonin receptor modulator is risperidone and the psychedelic is 1P-LSD, wherein the risperidone is administered to post-treat at least 210 minutes post to the 1P-LSD. In some embodiments, the serotonin receptor modulator is risperidone and the psychedelic is 1P-LSD, wherein the risperidone is administered to post-treat at least 240 minutes post to the 1P-LSD. In some embodiments, the serotonin receptor modulator is risperidone and the psychedelic is 1P-LSD, wherein the risperidone is administered to post-treat at least 270 minutes post to the 1P-LSD. In some embodiments, the serotonin receptor modulator is risperidone and the psychedelic is 1P-LSD, wherein the risperidone is administered to post-treat at least 300 minutes post to the 1P-LSD. In some embodiments, the serotonin receptor modulator is risperidone and the psychedelic is 1P-LSD, wherein the risperidone is administered to post-treat at least 330 minutes post to the 1P-LSD. In some embodiments, the serotonin receptor modulator is risperidone and the psychedelic is 1P-LSD, wherein the risperidone is administered to post-treat at least 360 minutes post to the 1P-LSD. In some preferred embodiments, the serotonin receptor modulator is risperidone and the psychedelic is 1P-LSD, wherein risperidone is administered to post-treat between about 60 minutes and about 180 minutes post to the administration of 1P-LSD.

In some embodiments, the serotonin receptor modulator is eplivanserin and the psychedelic is DMT, wherein the eplivanserin is administered to post-treat at least 15 minutes post to the administration of DMT. In some embodiments, the serotonin receptor modulator is eplivanserin and the psychedelic is DMT, wherein the eplivanserin is administered to post-treat at least 30 minutes post to the DMT. In some embodiments, the serotonin receptor modulator is eplivanserin and the psychedelic is DMT, wherein the eplivanserin is administered to post-treat at least 60 minutes post to the DMT. In some embodiments, the serotonin receptor modulator is eplivanserin and the psychedelic is DMT, wherein the eplivanserin is administered to post-treat at least 90 minutes post to the DMT. In some embodiments, the serotonin receptor modulator is eplivanserin and the psychedelic is DMT, wherein the eplivanserin is administered to post-treat at least 120 minutes post to the DMT. In some embodiments, the serotonin receptor modulator is eplivanserin and the psychedelic is DMT, wherein the eplivanserin is administered to post-treat at least 150 minutes post to the DMT. In some embodiments, the serotonin receptor modulator is eplivanserin and the psychedelic is DMT, wherein the eplivanserin is administered to post-treat between about 15 minutes and about 150 minutes post to the DMT. In some embodiments, the serotonin receptor modulator is eplivanserin and the psychedelic is DMT, wherein the eplivanserin is administered to post-treat at least 180 minutes post to the DMT. In some embodiments, the serotonin receptor modulator is eplivanserin and the psychedelic is DMT, wherein the eplivanserin is administered to post-treat at least 210 minutes post to the DMT. In some embodiments, the serotonin receptor modulator is eplivanserin and the psychedelic is DMT, wherein the eplivanserin is administered to post-treat at least 240 minutes post to the DMT. In some embodiments, the serotonin receptor modulator is eplivanserin and the psychedelic is DMT, wherein the eplivanserin is administered to post-treat at least 270 minutes post to the DMT. In some embodiments, the serotonin receptor modulator is eplivanserin and the psychedelic is DMT, wherein the eplivanserin is administered to post-treat at least 300 minutes post to the DMT. In some embodiments, the serotonin receptor modulator is eplivanserin and the psychedelic is DMT, wherein the eplivanserin is administered to post-treat at least 330 minutes post to the DMT. In some embodiments, the serotonin receptor modulator is eplivanserin and the psychedelic is DMT, wherein the eplivanserin is administered to post-treat at least 360 minutes post to the DMT.

In some preferred embodiments, the serotonin receptor modulator is eplivanserin and the psychedelic is DMT, wherein eplivanserin is administered to post-treat between about 60 minutes and about 180 minutes post to the administration of DMT.

In some embodiments, the serotonin receptor modulator is volinanserin and the psychedelic is DMT, wherein the volinanserin is administered to post-treat at least 15 minutes post to the administration of DMT. In some embodiments, the serotonin receptor modulator is volinanserin and the psychedelic is DMT, wherein the volinanserin is administered to post-treat at least 30 minutes post to the DMT. In some embodiments, the serotonin receptor modulator is volinanserin and the psychedelic is DMT, wherein the volinanserin is administered to post-treat at least 60 minutes post to the DMT. In some embodiments, the serotonin receptor modulator is volinanserin and the psychedelic is DMT, wherein the volinanserin is administered to post-treat at least 90 minutes post to the DMT. In some embodiments, the serotonin receptor modulator is volinanserin and the psychedelic is DMT, wherein the volinanserin is administered to post-treat at least 120 minutes post to the DMT. In some embodiments, the serotonin receptor modulator is volinanserin and the psychedelic is DMT, wherein the volinanserin is administered to post-treat at least 150 minutes post to the DMT. In some embodiments, the serotonin receptor modulator is volinanserin and the psychedelic is DMT, wherein the volinanserin is administered to post-treat between about 15 minutes and about 150 minutes post to the DMT. In some embodiments, the serotonin receptor modulator is volinanserin and the psychedelic is DMT, wherein the volinanserin is administered to post-treat at least 180 minutes post to the DMT. In some embodiments, the serotonin receptor modulator is volinanserin and the psychedelic is DMT, wherein the volinanserin is administered to post-treat at least 210 minutes post to the DMT. In some embodiments, the serotonin receptor modulator is volinanserin and the psychedelic is DMT, wherein the volinanserin is administered to post-treat at least 240 minutes post to the DMT. In some embodiments, the serotonin receptor modulator is volinanserin and the psychedelic is DMT, wherein the volinanserin is administered to post-treat at least 270 minutes post to the DMT. In some embodiments, the serotonin receptor modulator is volinanserin and the psychedelic is DMT, wherein the volinanserin is administered to post-treat at least 300 minutes post to the DMT. In some embodiments, the serotonin receptor modulator is volinanserin and the psychedelic is DMT, wherein the volinanserin is administered to post-treat at least 330 minutes post to the DMT. In some embodiments, the serotonin receptor modulator is volinanserin and the psychedelic is DMT, wherein the volinanserin is administered to post-treat at least 360 minutes post to the DMT. In some preferred embodiments, the serotonin receptor modulator is volinanserin and the psychedelic is DMT, wherein volinanserin is administered to post-treat between about 60 minutes and about 180 minutes post to the administration of DMT.

In some embodiments, the serotonin receptor modulator is ketanserin and the psychedelic is DMT, wherein the ketanserin is administered to post-treat at least 15 minutes post to the administration of DMT. In some embodiments, the serotonin receptor modulator is ketanserin and the psychedelic is DMT, wherein the ketanserin is administered to post-treat at least 30 minutes post to the DMT. In some embodiments, the serotonin receptor modulator is ketanserin and the psychedelic is DMT, wherein the ketanserin is administered to post-treat at least 60 minutes post to the DMT. In some embodiments, the serotonin receptor modulator is ketanserin and the psychedelic is DMT, wherein the ketanserin is administered to post-treat at least 90 minutes post to the DMT. In some embodiments, the serotonin receptor modulator is ketanserin and the psychedelic is DMT, wherein the ketanserin is administered to post-treat at least 120 minutes post to the DMT. In some embodiments, the serotonin receptor modulator is ketanserin and the psychedelic is DMT, wherein the ketanserin is administered to post-treat at least 150 minutes post to the DMT. In some embodiments, the serotonin receptor modulator is ketanserin and the psychedelic is DMT, wherein the ketanserin is administered to post-treat between about 15 minutes and about 150 minutes post to the DMT. In some embodiments, the serotonin receptor modulator is ketanserin and the psychedelic is DMT, wherein the ketanserin is administered to post-treat at least 180 minutes post to the DMT. In some embodiments, the serotonin receptor modulator is ketanserin and the psychedelic is DMT, wherein the ketanserin is administered to post-treat at least 210 minutes post to the DMT. In some embodiments, the serotonin receptor modulator is ketanserin and the psychedelic is DMT, wherein the ketanserin is administered to post-treat at least 240 minutes post to the DMT. In some embodiments, the serotonin receptor modulator is ketanserin and the psychedelic is DMT, wherein the ketanserin is administered to post-treat at least 270 minutes post to the DMT. In some embodiments, the serotonin receptor modulator is ketanserin and the psychedelic is DMT, wherein the ketanserin is administered to post-treat at least 300 minutes post to the DMT. In some embodiments, the serotonin receptor modulator is ketanserin and the psychedelic is DMT, wherein the ketanserin is administered to post-treat at least 330 minutes post to the DMT. In some embodiments, the serotonin receptor modulator is ketanserin and the psychedelic is DMT, wherein the ketanserin is administered to post-treat at least 360 minutes post to the DMT. In some preferred embodiments, the serotonin receptor modulator is ketanserin and the psychedelic is DMT, wherein ketanserin is administered to post-treat between about 60 minutes and about 180 minutes post to the administration of DMT.

In some embodiments, the serotonin receptor modulator is ritanserin and the psychedelic is DMT, wherein the ritanserin is administered to post-treat at least 15 minutes post to the DMT. In some embodiments, the serotonin receptor modulator is ritanserin and the psychedelic is DMT, wherein the ritanserin is administered to post-treat at least 30 minutes post to the DMT. In some embodiments, the serotonin receptor modulator is ritanserin and the psychedelic is DMT, wherein the ritanserin is administered to post-treat at least 60 minutes post to the DMT. In some embodiments, the serotonin receptor modulator is ritanserin and the psychedelic is DMT, wherein the ritanserin is administered to post-treat at least 90 minutes post to the DMT. In some embodiments, the serotonin receptor modulator is ritanserin and the psychedelic is DMT, wherein the ritanserin is administered to post-treat at least 120 minutes post to the DMT. In some embodiments, the serotonin receptor modulator is ritanserin and the psychedelic is DMT, wherein the ritanserin is administered to post-treat at least 150 minutes post to the DMT. In some embodiments, the serotonin receptor modulator is ritanserin and the psychedelic is DMT, wherein the ritanserin is administered to post-treat between about 15 minutes and about 150 minutes post to the DMT. In some embodiments, the serotonin receptor modulator is ritanserin and the psychedelic is DMT, wherein the ritanserin is administered to post-treat at least 180 minutes post to the DMT. In some embodiments, the serotonin receptor modulator is ritanserin and the psychedelic is DMT, wherein the ritanserin is administered to post-treat at least 210 minutes post to the DMT. In some embodiments, the serotonin receptor modulator is ritanserin and the psychedelic is DMT, wherein the ritanserin is administered to post-treat at least 240 minutes post to the DMT. In some embodiments, the serotonin receptor modulator is ritanserin and the psychedelic is DMT, wherein the ritanserin is administered to post-treat at least 270 minutes post to the DMT. In some embodiments, the serotonin receptor modulator is ritanserin and the psychedelic is DMT, wherein the ritanserin is administered to post-treat at least 300 minutes post to the DMT. In some embodiments, the serotonin receptor modulator is ritanserin and the psychedelic is DMT, wherein the ritanserin is administered to post-treat at least 330 minutes post to the DMT. In some embodiments, the serotonin receptor modulator is ritanserin and the psychedelic is DMT, wherein the ritanserin is administered to post-treat at least 360 minutes post to the DMT. In some preferred embodiments, the serotonin receptor modulator is ritanserin and the psychedelic is DMT, wherein ritanserin is administered to post-treat between about 60 minutes and about 180 minutes post to the administration of DMT.

In some embodiments, the serotonin receptor modulator is pimavanserin and the psychedelic is DMT, wherein the pimavanserin is administered to post-treat at least 15 minutes post to the DMT. In some embodiments, the serotonin receptor modulator is pimavanserin and the psychedelic is DMT, wherein the pimavanserin is administered to post-treat at least 30 minutes post to the DMT. In some embodiments, the serotonin receptor modulator is pimavanserin and the psychedelic is DMT, wherein the pimavanserin is administered to post-treat at least 60 minutes post to the DMT. In some embodiments, the serotonin receptor modulator is pimavanserin and the psychedelic is DMT, wherein the pimavanserin is administered to post-treat at least 90 minutes post to the DMT. In some embodiments, the serotonin receptor modulator is pimavanserin and the psychedelic is DMT, wherein the pimavanserin is administered to post-treat at least 120 minutes post to the DMT. In some embodiments, the serotonin receptor modulator is pimavanserin and the psychedelic is DMT, wherein the pimavanserin is administered to post-treat at least 150 minutes post to the DMT. In some embodiments, the serotonin receptor modulator is pimavanserin and the psychedelic is DMT, wherein the pimavanserin is administered to post-treat between about 15 minutes and about 150 minutes post to the DMT. In some embodiments, the serotonin receptor modulator is pimavanserin and the psychedelic is DMT, wherein the pimavanserin is administered to post-treat at least 180 minutes post to the DMT. In some embodiments, the serotonin receptor modulator is pimavanserin and the psychedelic is DMT, wherein the pimavanserin is administered to post-treat at least 210 minutes post to the DMT. In some embodiments, the serotonin receptor modulator is pimavanserin and the psychedelic is DMT, wherein the pimavanserin is administered to post-treat at least 240 minutes post to the DMT. In some embodiments, the serotonin receptor modulator is pimavanserin and the psychedelic is DMT, wherein the pimavanserin is administered to post-treat at least 270 minutes post to the DMT. In some embodiments, the serotonin receptor modulator is pimavanserin and the psychedelic is DMT, wherein the pimavanserin is administered to post-treat at least 300 minutes post to the DMT. In some embodiments, the serotonin receptor modulator is pimavanserin and the psychedelic is DMT, wherein the pimavanserin is administered to post-treat at least 330 minutes post to the DMT. In some embodiments, the serotonin receptor modulator is pimavanserin and the psychedelic is DMT, wherein the pimavanserin is administered to post-treat at least 360 minutes post to the DMT. In some preferred embodiments, the serotonin receptor modulator is pimavanserin and the psychedelic is DMT, wherein pimavanserin is administered to post-treat between about 60 minutes and about 180 minutes post to the administration of DMT.

In some embodiments, the serotonin receptor modulator is nelotanserin and the psychedelic is DMT, wherein the nelotanserin is administered to post-treat at least 15 minutes post to the DMT. In some embodiments, the serotonin receptor modulator is nelotanserin and the psychedelic is DMT, wherein the nelotanserin is administered to post-treat at least 30 minutes post to the DMT. In some embodiments, the serotonin receptor modulator is nelotanserin and the psychedelic is DMT, wherein the nelotanserin is administered to post-treat at least 60 minutes post to the DMT. In some embodiments, the serotonin receptor modulator is nelotanserin and the psychedelic is DMT, wherein the nelotanserin is administered to post-treat at least 90 minutes post to the DMT. In some embodiments, the serotonin receptor modulator is nelotanserin and the psychedelic is DMT, wherein the nelotanserin is administered to post-treat at least 120 minutes post to the DMT. In some embodiments, the serotonin receptor modulator is nelotanserin and the psychedelic is DMT, wherein the nelotanserin is administered to post-treat at least 150 minutes post to the DMT. In some embodiments, the serotonin receptor modulator is nelotanserin and the psychedelic is DMT, wherein the nelotanserin is administered to post-treat between about 15 minutes and about 150 minutes post to the DMT. In some embodiments, the serotonin receptor modulator is nelotanserin and the psychedelic is DMT, wherein the nelotanserin is administered to post-treat at least 180 minutes post to the DMT. In some embodiments, the serotonin receptor modulator is nelotanserin and the psychedelic is DMT, wherein the nelotanserin is administered to post-treat at least 210 minutes post to the DMT. In some embodiments, the serotonin receptor modulator is nelotanserin and the psychedelic is DMT, wherein the nelotanserin is administered to post-treat at least 240 minutes post to the DMT. In some embodiments, the serotonin receptor modulator is nelotanserin and the psychedelic is DMT, wherein the nelotanserin is administered to post-treat at least 270 minutes post to the DMT. In some embodiments, the serotonin receptor modulator is nelotanserin and the psychedelic is DMT, wherein the nelotanserin is administered to post-treat at least 300 minutes post to the DMT. In some embodiments, the serotonin receptor modulator is nelotanserin and the psychedelic is DMT, wherein the nelotanserin is administered to post-treat at least 330 minutes post to the DMT. In some embodiments, the serotonin receptor modulator is nelotanserin and the psychedelic is DMT, wherein the nelotanserin is administered to post-treat at least 360 minutes post to the DMT. In some preferred embodiments, the serotonin receptor modulator is nelotanserin and the psychedelic is DMT, wherein nelotanserin is administered to post-treat between about 60 minutes and about 180 minutes post to the administration of DMT.

In some embodiments, the serotonin receptor modulator is pruvanserin and the psychedelic is DMT, wherein the pruvanserin is administered to post-treat at least 15 minutes post to the DMT. In some embodiments, the serotonin receptor modulator is pruvanserin and the psychedelic is DMT, wherein the pruvanserin is administered to post-treat at least 30 minutes post to the DMT. In some embodiments, the serotonin receptor modulator is pruvanserin and the psychedelic is DMT, wherein the pruvanserin is administered to post-treat at least 60 minutes post to the DMT. In some embodiments, the serotonin receptor modulator is pruvanserin and the psychedelic is DMT, wherein the pruvanserin is administered to post-treat at least 90 minutes post to the DMT. In some embodiments, the serotonin receptor modulator is pruvanserin and the psychedelic is DMT, wherein the pruvanserin is administered to post-treat at least 120 minutes post to the DMT. In some embodiments, the serotonin receptor modulator is pruvanserin and the psychedelic is DMT, wherein the pruvanserin is administered to post-treat at least 150 minutes post to the DMT. In some embodiments, the serotonin receptor modulator is pruvanserin and the psychedelic is DMT, wherein the pruvanserin is administered to post-treat between about 15 minutes and about 150 minutes post to the DMT. In some embodiments, the serotonin receptor modulator is pruvanserin and the psychedelic is DMT, wherein the pruvanserin is administered to post-treat at least 180 minutes post to the DMT. In some embodiments, the serotonin receptor modulator is pruvanserin and the psychedelic is DMT, wherein the pruvanserin is administered to post-treat at least 210 minutes post to the DMT. In some embodiments, the serotonin receptor modulator is pruvanserin and the psychedelic is DMT, wherein the pruvanserin is administered to post-treat at least 240 minutes post to the DMT. In some embodiments, the serotonin receptor modulator is pruvanserin and the psychedelic is DMT, wherein the pruvanserin is administered to post-treat at least 270 minutes post to the DMT. In some embodiments, the serotonin receptor modulator is pruvanserin and the psychedelic is DMT, wherein the pruvanserin is administered to post-treat at least 300 minutes post to the DMT. In some embodiments, the serotonin receptor modulator is pruvanserin and the psychedelic is DMT, wherein the pruvanserin is administered to post-treat at least 330 minutes post to the DMT. In some embodiments, the serotonin receptor modulator is pruvanserin and the psychedelic is DMT, wherein the pruvanserin is administered to post-treat at least 360 minutes post to the DMT. In some preferred embodiments, the serotonin receptor modulator is pruvanserin and the psychedelic is DMT, wherein pruvanserin is administered to post-treat between about 60 minutes and about 180 minutes post to the administration of DMT.

In some embodiments, the serotonin receptor modulator is flibanserin and the psychedelic is DMT, wherein the flibanserin is administered to post-treat at least 15 minutes post to the administration of DMT. In some embodiments, the serotonin receptor modulator is flibanserin and the psychedelic is DMT, wherein the flibanserin is administered to post-treat at least 30 minutes post to the DMT. In some embodiments, the serotonin receptor modulator is flibanserin and the psychedelic is DMT, wherein the flibanserin is administered to post-treat at least 60 minutes post to the DMT. In some embodiments, the serotonin receptor modulator is flibanserin and the psychedelic is DMT, wherein the flibanserin is administered to post-treat at least 90 minutes post to the DMT. In some embodiments, the serotonin receptor modulator is flibanserin and the psychedelic is DMT, wherein the flibanserin is administered to post-treat at least 120 minutes post to the DMT. In some embodiments, the serotonin receptor modulator is flibanserin and the psychedelic is DMT, wherein the flibanserin is administered to post-treat at least 150 minutes post to the DMT. In some embodiments, the serotonin receptor modulator is flibanserin and the psychedelic is DMT, wherein the flibanserin is administered to post-treat between about 15 minutes and about 150 minutes post to the DMT. In some embodiments, the serotonin receptor modulator is flibanserin and the psychedelic is DMT, wherein the flibanserin is administered to post-treat at least 180 minutes post to the DMT. In some embodiments, the serotonin receptor modulator is flibanserin and the psychedelic is DMT, wherein the flibanserin is administered to post-treat at least 210 minutes post to the DMT. In some embodiments, the serotonin receptor modulator is flibanserin and the psychedelic is DMT, wherein the flibanserin is administered to post-treat at least 240 minutes post to the DMT. In some embodiments, the serotonin receptor modulator is flibanserin and the psychedelic is DMT, wherein the flibanserin is administered to post-treat at least 270 minutes post to the DMT. In some embodiments, the serotonin receptor modulator is flibanserin and the psychedelic is DMT, wherein the flibanserin is administered to post-treat at least 300 minutes post to the DMT. In some embodiments, the serotonin receptor modulator is flibanserin and the psychedelic is DMT, wherein the flibanserin is administered to post-treat at least 330 minutes post to the DMT. In some embodiments, the serotonin receptor modulator is flibanserin and the psychedelic is DMT, wherein the flibanserin is administered to post-treat at least 360 minutes post to the DMT. In some preferred embodiments, the serotonin receptor modulator is flibanserin and the psychedelic is DMT, wherein flibanserin is administered to post-treat between about 60 minutes and about 180 minutes post to the administration of DMT.

In some embodiments, the serotonin receptor modulator is olanzapine and the psychedelic is DMT, wherein the olanzapine is administered to post-treat at least 15 minutes post to the administration of DMT. In some embodiments, the serotonin receptor modulator is olanzapine and the psychedelic is DMT, wherein the olanzapine is administered to post-treat at least 30 minutes post to the DMT. In some embodiments, the serotonin receptor modulator is olanzapine and the psychedelic is DMT, wherein the olanzapine is administered to post-treat at least 60 minutes post to the DMT. In some embodiments, the serotonin receptor modulator is olanzapine and the psychedelic is DMT, wherein the olanzapine is administered to post-treat at least 90 minutes post to the DMT. In some embodiments, the serotonin receptor modulator is olanzapine and the psychedelic is DMT, wherein the olanzapine is administered to post-treat at least 120 minutes post to the DMT. In some embodiments, the serotonin receptor modulator is olanzapine and the psychedelic is DMT, wherein the olanzapine is administered to post-treat at least 150 minutes post to the DMT. In some embodiments, the serotonin receptor modulator is olanzapine and the psychedelic is DMT, wherein the olanzapine is administered to post-treat between about 15 minutes and about 150 minutes post to the DMT. In some embodiments, the serotonin receptor modulator is olanzapine and the psychedelic is DMT, wherein the olanzapine is administered to post-treat at least 180 minutes post to the DMT. In some embodiments, the serotonin receptor modulator is olanzapine and the psychedelic is DMT, wherein the olanzapine is administered to post-treat at least 210 minutes post to the DMT. In some embodiments, the serotonin receptor modulator is olanzapine and the psychedelic is DMT, wherein the olanzapine is administered to post-treat at least 240 minutes post to the DMT. In some embodiments, the serotonin receptor modulator is olanzapine and the psychedelic is DMT, wherein the olanzapine is administered to post-treat at least 270 minutes post to the DMT. In some embodiments, the serotonin receptor modulator is olanzapine and the psychedelic is DMT, wherein the olanzapine is administered to post-treat at least 300 minutes post to the DMT. In some embodiments, the serotonin receptor modulator is olanzapine and the psychedelic is DMT, wherein the olanzapine is administered to post-treat at least 330 minutes post to the DMT. In some embodiments, the serotonin receptor modulator is olanzapine and the psychedelic is DMT, wherein the olanzapine is administered to post-treat at least 360 minutes post to the DMT. In some preferred embodiments, the serotonin receptor modulator is olanzapine and the psychedelic is DMT, wherein olanzapine is administered to post-treat between about 60 minutes and about 180 minutes post to the administration of DMT.

In some embodiments, the serotonin receptor modulator is quetiapine and the psychedelic is DMT, wherein the quetiapine is administered to post-treat at least 15 minutes post to the administration of DMT. In some embodiments, the serotonin receptor modulator is quetiapine and the psychedelic is DMT, wherein the quetiapine is administered to post-treat at least 30 minutes post to the DMT. In some embodiments, the serotonin receptor modulator is quetiapine and the psychedelic is DMT, wherein the quetiapine is administered to post-treat at least 60 minutes post to the DMT. In some embodiments, the serotonin receptor modulator is quetiapine and the psychedelic is DMT, wherein the quetiapine is administered to post-treat at least 90 minutes post to the DMT. In some embodiments, the serotonin receptor modulator is quetiapine and the psychedelic is DMT, wherein the quetiapine is administered to post-treat at least 120 minutes post to the DMT. In some embodiments, the serotonin receptor modulator is quetiapine and the psychedelic is DMT, wherein the quetiapine is administered to post-treat at least 150 minutes post to the DMT. In some embodiments, the serotonin receptor modulator is quetiapine and the psychedelic is DMT, wherein the quetiapine is administered to post-treat between about 15 minutes and about 150 minutes post to the DMT. In some embodiments, the serotonin receptor modulator is quetiapine and the psychedelic is DMT, wherein the quetiapine is administered to post-treat at least 180 minutes post to the DMT. In some embodiments, the serotonin receptor modulator is quetiapine and the psychedelic is DMT, wherein the quetiapine is administered to post-treat at least 210 minutes post to the DMT. In some embodiments, the serotonin receptor modulator is quetiapine and the psychedelic is DMT, wherein the quetiapine is administered to post-treat at least 240 minutes post to the DMT. In some embodiments, the serotonin receptor modulator is quetiapine and the psychedelic is DMT, wherein the quetiapine is administered to post-treat at least 270 minutes post to the DMT. In some embodiments, the serotonin receptor modulator is quetiapine and the psychedelic is DMT, wherein the quetiapine is administered to post-treat at least 300 minutes post to the DMT. In some embodiments, the serotonin receptor modulator is quetiapine and the psychedelic is DMT, wherein the quetiapine is administered to post-treat at least 330 minutes post to the DMT. In some embodiments, the serotonin receptor modulator is quetiapine and the psychedelic is DMT, wherein the quetiapine is administered to post-treat at least 360 minutes post to the DMT. In some preferred embodiments, the serotonin receptor modulator is quetiapine and the psychedelic is DMT, wherein quetiapine is administered to post-treat between about 60 minutes and about 180 minutes post to the administration of DMT.

In some embodiments, the serotonin receptor modulator is risperidone and the psychedelic is DMT, wherein the risperidone is administered to post-treat at least 15 minutes post to the administration of DMT. In some embodiments, the serotonin receptor modulator is risperidone and the psychedelic is DMT, wherein the risperidone is administered to post-treat at least 30 minutes post to the DMT. In some embodiments, the serotonin receptor modulator is risperidone and the psychedelic is DMT, wherein the risperidone is administered to post-treat at least 60 minutes post to the DMT. In some embodiments, the serotonin receptor modulator is risperidone and the psychedelic is DMT, wherein the risperidone is administered to post-treat at least 90 minutes post to the DMT. In some embodiments, the serotonin receptor modulator is risperidone and the psychedelic is DMT, wherein the risperidone is administered to post-treat at least 120 minutes post to the DMT. In some embodiments, the serotonin receptor modulator is risperidone and the psychedelic is DMT, wherein the risperidone is administered to post-treat at least 150 minutes post to the DMT. In some embodiments, the serotonin receptor modulator is risperidone and the psychedelic is DMT, wherein the risperidone is administered to post-treat between about 15 minutes and about 150 minutes post to the DMT. In some embodiments, the serotonin receptor modulator is risperidone and the psychedelic is DMT, wherein the risperidone is administered to post-treat at least 180 minutes post to the DMT. In some embodiments, the serotonin receptor modulator is risperidone and the psychedelic is DMT, wherein the risperidone is administered to post-treat at least 210 minutes post to the DMT. In some embodiments, the serotonin receptor modulator is risperidone and the psychedelic is DMT, wherein the risperidone is administered to post-treat at least 240 minutes post to the DMT. In some embodiments, the serotonin receptor modulator is risperidone and the psychedelic is DMT, wherein the risperidone is administered to post-treat at least 270 minutes post to the DMT. In some embodiments, the serotonin receptor modulator is risperidone and the psychedelic is DMT, wherein the risperidone is administered to post-treat at least 300 minutes post to the DMT. In some embodiments, the serotonin receptor modulator is risperidone and the psychedelic is DMT, wherein the risperidone is administered to post-treat at least 330 minutes post to the DMT. In some embodiments, the serotonin receptor modulator is risperidone and the psychedelic is DMT, wherein the risperidone is administered to post-treat at least 360 minutes post to the DMT. In some preferred embodiments, the serotonin receptor modulator is risperidone and the psychedelic is DMT, wherein risperidone is administered to post-treat between about 60 minutes and about 180 minutes post to the administration of DMT.

In some embodiments, the serotonin receptor modulator is eplivanserin and the psychedelic is 5-MeO-DMT, wherein the eplivanserin is administered to post-treat at least 15 minutes post to the administration of 5-MeO-DMT. In some embodiments, the serotonin receptor modulator is eplivanserin and the psychedelic is 5-MeO-DMT, wherein the eplivanserin is administered to post-treat at least 30 minutes post to the 5-MeO-DMT. In some embodiments, the serotonin receptor modulator is eplivanserin and the psychedelic is 5-MeO-DMT, wherein the eplivanserin is administered to post-treat at least 60 minutes post to the 5-MeO-DMT. In some embodiments, the serotonin receptor modulator is eplivanserin and the psychedelic is 5-MeO-DMT, wherein the eplivanserin is administered to post-treat at least 90 minutes post to the 5-MeO-DMT. In some embodiments, the serotonin receptor modulator is eplivanserin and the psychedelic is 5-MeO-DMT, wherein the eplivanserin is administered to post-treat at least 120 minutes post to the 5-MeO-DMT. In some embodiments, the serotonin receptor modulator is eplivanserin and the psychedelic is 5-MeO-DMT, wherein the eplivanserin is administered to post-treat at least 150 minutes post to the 5-MeO-DMT. In some embodiments, the serotonin receptor modulator is eplivanserin and the psychedelic is 5-MeO-DMT, wherein the eplivanserin is administered to post-treat between about 15 minutes and about 150 minutes post to the 5-MeO-DMT. In some embodiments, the serotonin receptor modulator is eplivanserin and the psychedelic is 5-MeO-DMT, wherein the eplivanserin is administered to post-treat at least 180 minutes post to the 5-MeO-DMT. In some embodiments, the serotonin receptor modulator is eplivanserin and the psychedelic is 5-MeO-DMT, wherein the eplivanserin is administered to post-treat at least 210 minutes post to the 5-MeO-DMT. In some embodiments, the serotonin receptor modulator is eplivanserin and the psychedelic is 5-MeO-DMT, wherein the eplivanserin is administered to post-treat at least 240 minutes post to the 5-MeO-DMT. In some embodiments, the serotonin receptor modulator is eplivanserin and the psychedelic is 5-MeO-DMT, wherein the eplivanserin is administered to post-treat at least 270 minutes post to the 5-MeO-DMT. In some embodiments, the serotonin receptor modulator is eplivanserin and the psychedelic is 5-MeO-DMT, wherein the eplivanserin is administered to post-treat at least 300 minutes post to the 5-MeO-DMT. In some embodiments, the serotonin receptor modulator is eplivanserin and the psychedelic is 5-MeO-DMT, wherein the eplivanserin is administered to post-treat at least 330 minutes post to the 5-MeO-DMT. In some embodiments, the serotonin receptor modulator is eplivanserin and the psychedelic is 5-MeO-DMT, wherein the eplivanserin is administered to post-treat at least 360 minutes post to the 5-MeO-DMT.

In some preferred embodiments, the serotonin receptor modulator is eplivanserin and the psychedelic is 5-MeO-DMT, wherein eplivanserin is administered to post-treat between about 60 minutes and about 180 minutes post to the administration of 5-MeO-DMT.

In some embodiments, the serotonin receptor modulator is volinanserin and the psychedelic is 5-MeO-DMT, wherein the volinanserin is administered to post-treat at least 15 minutes post to the administration of 5-MeO-DMT. In some embodiments, the serotonin receptor modulator is volinanserin and the psychedelic is 5-MeO-DMT, wherein the volinanserin is administered to post-treat at least 30 minutes post to the 5-MeO-DMT. In some embodiments, the serotonin receptor modulator is volinanserin and the psychedelic is 5-MeO-DMT, wherein the volinanserin is administered to post-treat at least 60 minutes post to the 5-MeO-DMT. In some embodiments, the serotonin receptor modulator is volinanserin and the psychedelic is 5-MeO-DMT, wherein the volinanserin is administered to post-treat at least 90 minutes post to the 5-MeO-DMT. In some embodiments, the serotonin receptor modulator is volinanserin and the psychedelic is 5-MeO-DMT, wherein the volinanserin is administered to post-treat at least 120 minutes post to the 5-MeO-DMT. In some embodiments, the serotonin receptor modulator is volinanserin and the psychedelic is 5-MeO-DMT, wherein the volinanserin is administered to post-treat at least 150 minutes post to the 5-MeO-DMT. In some embodiments, the serotonin receptor modulator is volinanserin and the psychedelic is 5-MeO-DMT, wherein the volinanserin is administered to post-treat between about 15 minutes and about 150 minutes post to the 5-MeO-DMT. In some embodiments, the serotonin receptor modulator is volinanserin and the psychedelic is 5-MeO-DMT, wherein the volinanserin is administered to post-treat at least 180 minutes post to the 5-MeO-DMT. In some embodiments, the serotonin receptor modulator is volinanserin and the psychedelic is 5-MeO-DMT, wherein the volinanserin is administered to post-treat at least 210 minutes post to the 5-MeO-DMT. In some embodiments, the serotonin receptor modulator is volinanserin and the psychedelic is 5-MeO-DMT, wherein the volinanserin is administered to post-treat at least 240 minutes post to the 5-MeO-DMT. In some embodiments, the serotonin receptor modulator is volinanserin and the psychedelic is 5-MeO-DMT, wherein the volinanserin is administered to post-treat at least 270 minutes post to the 5-MeO-DMT. In some embodiments, the serotonin receptor modulator is volinanserin and the psychedelic is 5-MeO-DMT, wherein the volinanserin is administered to post-treat at least 300 minutes post to the 5-MeO-DMT. In some embodiments, the serotonin receptor modulator is volinanserin and the psychedelic is 5-MeO-DMT, wherein the volinanserin is administered to post-treat at least 330 minutes post to the 5-MeO-DMT. In some embodiments, the serotonin receptor modulator is volinanserin and the psychedelic is 5-MeO-DMT, wherein the volinanserin is administered to post-treat at least 360 minutes post to the 5-MeO-DMT. In some preferred embodiments, the serotonin receptor modulator is volinanserin and the psychedelic is 5-MeO-DMT, wherein volinanserin is administered to post-treat between about 60 minutes and about 180 minutes post to the administration of 5-MeO-DMT.

In some embodiments, the serotonin receptor modulator is ketanserin and the psychedelic is 5-MeO-DMT, wherein the ketanserin is administered to post-treat at least 15 minutes post to the administration of 5-MeO-DMT. In some embodiments, the serotonin receptor modulator is ketanserin and the psychedelic is 5-MeO-DMT, wherein the ketanserin is administered to post-treat at least 30 minutes post to the 5-MeO-DMT. In some embodiments, the serotonin receptor modulator is ketanserin and the psychedelic is 5-MeO-DMT, wherein the ketanserin is administered to post-treat at least 60 minutes post to the 5-MeO-DMT. In some embodiments, the serotonin receptor modulator is ketanserin and the psychedelic is 5-MeO-DMT, wherein the ketanserin is administered to post-treat at least 90 minutes post to the 5-MeO-DMT. In some embodiments, the serotonin receptor modulator is ketanserin and the psychedelic is 5-MeO-DMT, wherein the ketanserin is administered to post-treat at least 120 minutes post to the 5-MeO-DMT. In some embodiments, the serotonin receptor modulator is ketanserin and the psychedelic is 5-MeO-DMT, wherein the ketanserin is administered to post-treat at least 150 minutes post to the 5-MeO-DMT. In some embodiments, the serotonin receptor modulator is ketanserin and the psychedelic is 5-MeO-DMT, wherein the ketanserin is administered to post-treat between about 15 minutes and about 150 minutes post to the 5-MeO-DMT. In some embodiments, the serotonin receptor modulator is ketanserin and the psychedelic is 5-MeO-DMT, wherein the ketanserin is administered to post-treat at least 180 minutes post to the 5-MeO-DMT. In some embodiments, the serotonin receptor modulator is ketanserin and the psychedelic is 5-MeO-DMT, wherein the ketanserin is administered to post-treat at least 210 minutes post to the 5-MeO-DMT. In some embodiments, the serotonin receptor modulator is ketanserin and the psychedelic is 5-MeO-DMT, wherein the ketanserin is administered to post-treat at least 240 minutes post to the 5-MeO-DMT. In some embodiments, the serotonin receptor modulator is ketanserin and the psychedelic is 5-MeO-DMT, wherein the ketanserin is administered to post-treat at least 270 minutes post to the 5-MeO-DMT. In some embodiments, the serotonin receptor modulator is ketanserin and the psychedelic is 5-MeO-DMT, wherein the ketanserin is administered to post-treat at least 300 minutes post to the 5-MeO-DMT. In some embodiments, the serotonin receptor modulator is ketanserin and the psychedelic is 5-MeO-DMT, wherein the ketanserin is administered to post-treat at least 330 minutes post to the 5-MeO-DMT. In some embodiments, the serotonin receptor modulator is ketanserin and the psychedelic is 5-MeO-DMT, wherein the ketanserin is administered to post-treat at least 360 minutes post to the 5-MeO-DMT. In some preferred embodiments, the serotonin receptor modulator is ketanserin and the psychedelic is 5-MeO-DMT, wherein ketanserin is administered to post-treat between about 60 minutes and about 180 minutes post to the administration of 5-MeO-DMT.

In some embodiments, the serotonin receptor modulator is ritanserin and the psychedelic is 5-MeO-DMT, wherein the ritanserin is administered to post-treat at least 15 minutes post to the 5-MeO-DMT. In some embodiments, the serotonin receptor modulator is ritanserin and the psychedelic is 5-MeO-DMT, wherein the ritanserin is administered to post-treat at least 30 minutes post to the 5-MeO-DMT. In some embodiments, the serotonin receptor modulator is ritanserin and the psychedelic is 5-MeO-DMT, wherein the ritanserin is administered to post-treat at least 60 minutes post to the 5-MeO-DMT. In some embodiments, the serotonin receptor modulator is ritanserin and the psychedelic is 5-MeO-DMT, wherein the ritanserin is administered to post-treat at least 90 minutes post to the 5-MeO-DMT. In some embodiments, the serotonin receptor modulator is ritanserin and the psychedelic is 5-MeO-DMT, wherein the ritanserin is administered to post-treat at least 120 minutes post to the 5-MeO-DMT. In some embodiments, the serotonin receptor modulator is ritanserin and the psychedelic is 5-MeO-DMT, wherein the ritanserin is administered to post-treat at least 150 minutes post to the 5-MeO-DMT. In some embodiments, the serotonin receptor modulator is ritanserin and the psychedelic is 5-MeO-DMT, wherein the ritanserin is administered to post-treat between about 15 minutes and about 150 minutes post to the 5-MeO-DMT. In some embodiments, the serotonin receptor modulator is ritanserin and the psychedelic is 5-MeO-DMT, wherein the ritanserin is administered to post-treat at least 180 minutes post to the 5-MeO-DMT. In some embodiments, the serotonin receptor modulator is ritanserin and the psychedelic is 5-MeO-DMT, wherein the ritanserin is administered to post-treat at least 210 minutes post to the 5-MeO-DMT. In some embodiments, the serotonin receptor modulator is ritanserin and the psychedelic is 5-MeO-DMT, wherein the ritanserin is administered to post-treat at least 240 minutes post to the 5-MeO-DMT. In some embodiments, the serotonin receptor modulator is ritanserin and the psychedelic is 5-MeO-DMT, wherein the ritanserin is administered to post-treat at least 270 minutes post to the 5-MeO-DMT. In some embodiments, the serotonin receptor modulator is ritanserin and the psychedelic is 5-MeO-DMT, wherein the ritanserin is administered to post-treat at least 300 minutes post to the 5-MeO-DMT. In some embodiments, the serotonin receptor modulator is ritanserin and the psychedelic is 5-MeO-DMT, wherein the ritanserin is administered to post-treat at least 330 minutes post to the 5-MeO-DMT. In some embodiments, the serotonin receptor modulator is ritanserin and the psychedelic is 5-MeO-DMT, wherein the ritanserin is administered to post-treat at least 360 minutes post to the 5-MeO-DMT. In some preferred embodiments, the serotonin receptor modulator is ritanserin and the psychedelic is 5-MeO-DMT, wherein ritanserin is administered to post-treat between about 60 minutes and about 180 minutes post to the administration of 5-MeO-DMT.

In some embodiments, the serotonin receptor modulator is pimavanserin and the psychedelic is 5-MeO-DMT, wherein the pimavanserin is administered to post-treat at least 15 minutes post to the 5-MeO-DMT. In some embodiments, the serotonin receptor modulator is pimavanserin and the psychedelic is 5-MeO-DMT, wherein the pimavanserin is administered to post-treat at least 30 minutes post to the 5-MeO-DMT. In some embodiments, the serotonin receptor modulator is pimavanserin and the psychedelic is 5-MeO-DMT, wherein the pimavanserin is administered to post-treat at least 60 minutes post to the 5-MeO-DMT. In some embodiments, the serotonin receptor modulator is pimavanserin and the psychedelic is 5-MeO-DMT, wherein the pimavanserin is administered to post-treat at least 90 minutes post to the 5-MeO-DMT. In some embodiments, the serotonin receptor modulator is pimavanserin and the psychedelic is 5-MeO-DMT, wherein the pimavanserin is administered to post-treat at least 120 minutes post to the 5-MeO-DMT. In some embodiments, the serotonin receptor modulator is pimavanserin and the psychedelic is 5-MeO-DMT, wherein the pimavanserin is administered to post-treat at least 150 minutes post to the 5-MeO-DMT. In some embodiments, the serotonin receptor modulator is pimavanserin and the psychedelic is 5-MeO-DMT, wherein the pimavanserin is administered to post-treat between about 15 minutes and about 150 minutes post to the 5-MeO-DMT. In some embodiments, the serotonin receptor modulator is pimavanserin and the psychedelic is 5-MeO-DMT, wherein the pimavanserin is administered to post-treat at least 180 minutes post to the 5-MeO-DMT. In some embodiments, the serotonin receptor modulator is pimavanserin and the psychedelic is 5-MeO-DMT, wherein the pimavanserin is administered to post-treat at least 210 minutes post to the 5-MeO-DMT. In some embodiments, the serotonin receptor modulator is pimavanserin and the psychedelic is 5-MeO-DMT, wherein the pimavanserin is administered to post-treat at least 240 minutes post to the 5-MeO-DMT. In some embodiments, the serotonin receptor modulator is pimavanserin and the psychedelic is 5-MeO-DMT, wherein the pimavanserin is administered to post-treat at least 270 minutes post to the 5-MeO-DMT. In some embodiments, the serotonin receptor modulator is pimavanserin and the psychedelic is 5-MeO-DMT, wherein the pimavanserin is administered to post-treat at least 300 minutes post to the 5-MeO-DMT. In some embodiments, the serotonin receptor modulator is pimavanserin and the psychedelic is 5-MeO-DMT, wherein the pimavanserin is administered to post-treat at least 330 minutes post to the 5-MeO-DMT. In some embodiments, the serotonin receptor modulator is pimavanserin and the psychedelic is 5-MeO-DMT, wherein the pimavanserin is administered to post-treat at least 360 minutes post to the 5-MeO-DMT. In some preferred embodiments, the serotonin receptor modulator is pimavanserin and the psychedelic is 5-MeO-DMT, wherein pimavanserin is administered to post-treat between about 60 minutes and about 180 minutes post to the administration of 5-MeO-DMT.

In some embodiments, the serotonin receptor modulator is nelotanserin and the psychedelic is 5-MeO-DMT, wherein the nelotanserin is administered to post-treat at least 15 minutes post to the 5-MeO-DMT. In some embodiments, the serotonin receptor modulator is nelotanserin and the psychedelic is 5-MeO-DMT, wherein the nelotanserin is administered to post-treat at least 30 minutes post to the 5-MeO-DMT. In some embodiments, the serotonin receptor modulator is nelotanserin and the psychedelic is 5-MeO-DMT, wherein the nelotanserin is administered to post-treat at least 60 minutes post to the 5-MeO-DMT. In some embodiments, the serotonin receptor modulator is nelotanserin and the psychedelic is 5-MeO-DMT, wherein the nelotanserin is administered to post-treat at least 90 minutes post to the 5-MeO-DMT. In some embodiments, the serotonin receptor modulator is nelotanserin and the psychedelic is 5-MeO-DMT, wherein the nelotanserin is administered to post-treat at least 120 minutes post to the 5-MeO-DMT. In some embodiments, the serotonin receptor modulator is nelotanserin and the psychedelic is 5-MeO-DMT, wherein the nelotanserin is administered to post-treat at least 150 minutes post to the 5-MeO-DMT. In some embodiments, the serotonin receptor modulator is nelotanserin and the psychedelic is 5-MeO-DMT, wherein the nelotanserin is administered to post-treat between about 15 minutes and about 150 minutes post to the 5-MeO-DMT. In some embodiments, the serotonin receptor modulator is nelotanserin and the psychedelic is 5-MeO-DMT, wherein the nelotanserin is administered to post-treat at least 180 minutes post to the 5-MeO-DMT. In some embodiments, the serotonin receptor modulator is nelotanserin and the psychedelic is 5-MeO-DMT, wherein the nelotanserin is administered to post-treat at least 210 minutes post to the 5-MeO-DMT. In some embodiments, the serotonin receptor modulator is nelotanserin and the psychedelic is 5-MeO-DMT, wherein the nelotanserin is administered to post-treat at least 240 minutes post to the 5-MeO-DMT. In some embodiments, the serotonin receptor modulator is nelotanserin and the psychedelic is 5-MeO-DMT, wherein the nelotanserin is administered to post-treat at least 270 minutes post to the 5-MeO-DMT. In some embodiments, the serotonin receptor modulator is nelotanserin and the psychedelic is 5-MeO-DMT, wherein the nelotanserin is administered to post-treat at least 300 minutes post to the 5-MeO-DMT. In some embodiments, the serotonin receptor modulator is nelotanserin and the psychedelic is 5-MeO-DMT, wherein the nelotanserin is administered to post-treat at least 330 minutes post to the 5-MeO-DMT. In some embodiments, the serotonin receptor modulator is nelotanserin and the psychedelic is 5-MeO-DMT, wherein the nelotanserin is administered to post-treat at least 360 minutes post to the 5-MeO-DMT. In some preferred embodiments, the serotonin receptor modulator is nelotanserin and the psychedelic is 5-MeO-DMT, wherein nelotanserin is administered to post-treat between about 60 minutes and about 180 minutes post to the administration of 5-MeO-DMT.

In some embodiments, the serotonin receptor modulator is pruvanserin and the psychedelic is 5-MeO-DMT, wherein the pruvanserin is administered to post-treat at least 15 minutes post to the 5-MeO-DMT. In some embodiments, the serotonin receptor modulator is pruvanserin and the psychedelic is 5-MeO-DMT, wherein the pruvanserin is administered to post-treat at least 30 minutes post to the 5-MeO-DMT. In some embodiments, the serotonin receptor modulator is pruvanserin and the psychedelic is 5-MeO-DMT, wherein the pruvanserin is administered to post-treat at least 60 minutes post to the 5-MeO-DMT. In some embodiments, the serotonin receptor modulator is pruvanserin and the psychedelic is 5-MeO-DMT, wherein the pruvanserin is administered to post-treat at least 90 minutes post to the 5-MeO-DMT. In some embodiments, the serotonin receptor modulator is pruvanserin and the psychedelic is 5-MeO-DMT, wherein the pruvanserin is administered to post-treat at least 120 minutes post to the 5-MeO-DMT. In some embodiments, the serotonin receptor modulator is pruvanserin and the psychedelic is 5-MeO-DMT, wherein the pruvanserin is administered to post-treat at least 150 minutes post to the 5-MeO-DMT. In some embodiments, the serotonin receptor modulator is pruvanserin and the psychedelic is 5-MeO-DMT, wherein the pruvanserin is administered to post-treat between about 15 minutes and about 150 minutes post to the 5-MeO-DMT. In some embodiments, the serotonin receptor modulator is pruvanserin and the psychedelic is 5-MeO-DMT, wherein the pruvanserin is administered to post-treat at least 180 minutes post to the 5-MeO-DMT. In some embodiments, the serotonin receptor modulator is pruvanserin and the psychedelic is 5-MeO-DMT, wherein the pruvanserin is administered to post-treat at least 210 minutes post to the 5-MeO-DMT. In some embodiments, the serotonin receptor modulator is pruvanserin and the psychedelic is 5-MeO-DMT, wherein the pruvanserin is administered to post-treat at least 240 minutes post to the 5-MeO-DMT. In some embodiments, the serotonin receptor modulator is pruvanserin and the psychedelic is 5-MeO-DMT, wherein the pruvanserin is administered to post-treat at least 270 minutes post to the 5-MeO-DMT. In some embodiments, the serotonin receptor modulator is pruvanserin and the psychedelic is 5-MeO-DMT, wherein the pruvanserin is administered to post-treat at least 300 minutes post to the 5-MeO-DMT. In some embodiments, the serotonin receptor modulator is pruvanserin and the psychedelic is 5-MeO-DMT, wherein the pruvanserin is administered to post-treat at least 330 minutes post to the 5-MeO-DMT. In some embodiments, the serotonin receptor modulator is pruvanserin and the psychedelic is 5-MeO-DMT, wherein the pruvanserin is administered to post-treat at least 360 minutes post to the 5-MeO-DMT. In some preferred embodiments, the serotonin receptor modulator is pruvanserin and the psychedelic is 5-MeO-DMT, wherein pruvanserin is administered to post-treat between about 60 minutes and about 180 minutes post to the administration of 5-MeO-DMT.

In some embodiments, the serotonin receptor modulator is flibanserin and the psychedelic is 5-MeO-DMT, wherein the flibanserin is administered to post-treat at least 15 minutes post to the administration of 5-MeO-DMT. In some embodiments, the serotonin receptor modulator is flibanserin and the psychedelic is 5-MeO-DMT, wherein the flibanserin is administered to post-treat at least 30 minutes post to the 5-MeO-DMT. In some embodiments, the serotonin receptor modulator is flibanserin and the psychedelic is 5-MeO-DMT, wherein the flibanserin is administered to post-treat at least 60 minutes post to the 5-MeO-DMT. In some embodiments, the serotonin receptor modulator is flibanserin and the psychedelic is 5-MeO-DMT, wherein the flibanserin is administered to post-treat at least 90 minutes post to the 5-MeO-DMT. In some embodiments, the serotonin receptor modulator is flibanserin and the psychedelic is 5-MeO-DMT, wherein the flibanserin is administered to post-treat at least 120 minutes post to the 5-MeO-DMT. In some embodiments, the serotonin receptor modulator is flibanserin and the psychedelic is 5-MeO-DMT, wherein the flibanserin is administered to post-treat at least 150 minutes post to the 5-MeO-DMT. In some embodiments, the serotonin receptor modulator is flibanserin and the psychedelic is 5-MeO-DMT, wherein the flibanserin is administered to post-treat between about 15 minutes and about 150 minutes post to the 5-MeO-DMT. In some embodiments, the serotonin receptor modulator is flibanserin and the psychedelic is 5-MeO-DMT, wherein the flibanserin is administered to post-treat at least 180 minutes post to the 5-MeO-DMT. In some embodiments, the serotonin receptor modulator is flibanserin and the psychedelic is 5-MeO-DMT, wherein the flibanserin is administered to post-treat at least 210 minutes post to the 5-MeO-DMT. In some embodiments, the serotonin receptor modulator is flibanserin and the psychedelic is 5-MeO-DMT, wherein the flibanserin is administered to post-treat at least 240 minutes post to the 5-MeO-DMT. In some embodiments, the serotonin receptor modulator is flibanserin and the psychedelic is 5-MeO-DMT, wherein the flibanserin is administered to post-treat at least 270 minutes post to the 5-MeO-DMT. In some embodiments, the serotonin receptor modulator is flibanserin and the psychedelic is 5-MeO-DMT, wherein the flibanserin is administered to post-treat at least 300 minutes post to the 5-MeO-DMT. In some embodiments, the serotonin receptor modulator is flibanserin and the psychedelic is 5-MeO-DMT, wherein the flibanserin is administered to post-treat at least 330 minutes post to the 5-MeO-DMT. In some embodiments, the serotonin receptor modulator is flibanserin and the psychedelic is 5-MeO-DMT, wherein the flibanserin is administered to post-treat at least 360 minutes post to the 5-MeO-DMT. In some preferred embodiments, the serotonin receptor modulator is flibanserin and the psychedelic is 5-MeO-DMT, wherein flibanserin is administered to post-treat between about 60 minutes and about 180 minutes post to the administration of 5-MeO-DMT.

In some embodiments, the serotonin receptor modulator is olanzapine and the psychedelic is 5-MeO-DMT, wherein the olanzapine is administered to post-treat at least 15 minutes post to the administration of 5-MeO-DMT. In some embodiments, the serotonin receptor modulator is olanzapine and the psychedelic is 5-MeO-DMT, wherein the olanzapine is administered to post-treat at least 30 minutes post to the 5-MeO-DMT. In some embodiments, the serotonin receptor modulator is olanzapine and the psychedelic is 5-MeO-DMT, wherein the olanzapine is administered to post-treat at least 60 minutes post to the 5-MeO-DMT. In some embodiments, the serotonin receptor modulator is olanzapine and the psychedelic is 5-MeO-DMT, wherein the olanzapine is administered to post-treat at least 90 minutes post to the 5-MeO-DMT. In some embodiments, the serotonin receptor modulator is olanzapine and the psychedelic is 5-MeO-DMT, wherein the olanzapine is administered to post-treat at least 120 minutes post to the 5-MeO-DMT. In some embodiments, the serotonin receptor modulator is olanzapine and the psychedelic is 5-MeO-DMT, wherein the olanzapine is administered to post-treat at least 150 minutes post to the 5-MeO-DMT. In some embodiments, the serotonin receptor modulator is olanzapine and the psychedelic is 5-MeO-DMT, wherein the olanzapine is administered to post-treat between about 15 minutes and about 150 minutes post to the 5-MeO-DMT. In some embodiments, the serotonin receptor modulator is olanzapine and the psychedelic is 5-MeO-DMT, wherein the olanzapine is administered to post-treat at least 180 minutes post to the 5-MeO-DMT. In some embodiments, the serotonin receptor modulator is olanzapine and the psychedelic is 5-MeO-DMT, wherein the olanzapine is administered to post-treat at least 210 minutes post to the 5-MeO-DMT. In some embodiments, the serotonin receptor modulator is olanzapine and the psychedelic is 5-MeO-DMT, wherein the olanzapine is administered to post-treat at least 240 minutes post to the 5-MeO-DMT. In some embodiments, the serotonin receptor modulator is olanzapine and the psychedelic is 5-MeO-DMT, wherein the olanzapine is administered to post-treat at least 270 minutes post to the 5-MeO-DMT. In some embodiments, the serotonin receptor modulator is olanzapine and the psychedelic is 5-MeO-DMT, wherein the olanzapine is administered to post-treat at least 300 minutes post to the 5-MeO-DMT. In some embodiments, the serotonin receptor modulator is olanzapine and the psychedelic is 5-MeO-DMT, wherein the olanzapine is administered to post-treat at least 330 minutes post to the 5-MeO-DMT. In some embodiments, the serotonin receptor modulator is olanzapine and the psychedelic is 5-MeO-DMT, wherein the olanzapine is administered to post-treat at least 360 minutes post to the 5-MeO-DMT. In some preferred embodiments, the serotonin receptor modulator is olanzapine and the psychedelic is 5-MeO-DMT, wherein olanzapine is administered to post-treat between about 60 minutes and about 180 minutes post to the administration of 5-MeO-DMT.

In some embodiments, the serotonin receptor modulator is quetiapine and the psychedelic is 5-MeO-DMT, wherein the quetiapine is administered to post-treat at least 15 minutes post to the administration of 5-MeO-DMT. In some embodiments, the serotonin receptor modulator is quetiapine and the psychedelic is 5-MeO-DMT, wherein the quetiapine is administered to post-treat at least 30 minutes post to the 5-MeO-DMT. In some embodiments, the serotonin receptor modulator is quetiapine and the psychedelic is 5-MeO-DMT, wherein the quetiapine is administered to post-treat at least 60 minutes post to the 5-MeO-DMT. In some embodiments, the serotonin receptor modulator is quetiapine and the psychedelic is 5-MeO-DMT, wherein the quetiapine is administered to post-treat at least 90 minutes post to the 5-MeO-DMT. In some embodiments, the serotonin receptor modulator is quetiapine and the psychedelic is 5-MeO-DMT, wherein the quetiapine is administered to post-treat at least 120 minutes post to the 5-MeO-DMT. In some embodiments, the serotonin receptor modulator is quetiapine and the psychedelic is 5-MeO-DMT, wherein the quetiapine is administered to post-treat at least 150 minutes post to the 5-MeO-DMT. In some embodiments, the serotonin receptor modulator is quetiapine and the psychedelic is 5-MeO-DMT, wherein the quetiapine is administered to post-treat between about 15 minutes and about 150 minutes post to the 5-MeO-DMT. In some embodiments, the serotonin receptor modulator is quetiapine and the psychedelic is 5-MeO-DMT, wherein the quetiapine is administered to post-treat at least 180 minutes post to the 5-MeO-DMT. In some embodiments, the serotonin receptor modulator is quetiapine and the psychedelic is 5-MeO-DMT, wherein the quetiapine is administered to post-treat at least 210 minutes post to the 5-MeO-DMT. In some embodiments, the serotonin receptor modulator is quetiapine and the psychedelic is 5-MeO-DMT, wherein the quetiapine is administered to post-treat at least 240 minutes post to the 5-MeO-DMT. In some embodiments, the serotonin receptor modulator is quetiapine and the psychedelic is 5-MeO-DMT, wherein the quetiapine is administered to post-treat at least 270 minutes post to the 5-MeO-DMT. In some embodiments, the serotonin receptor modulator is quetiapine and the psychedelic is 5-MeO-DMT, wherein the quetiapine is administered to post-treat at least 300 minutes post to the 5-MeO-DMT. In some embodiments, the serotonin receptor modulator is quetiapine and the psychedelic is 5-MeO-DMT, wherein the quetiapine is administered to post-treat at least 330 minutes post to the 5-MeO-DMT. In some embodiments, the serotonin receptor modulator is quetiapine and the psychedelic is 5-MeO-DMT, wherein the quetiapine is administered to post-treat at least 360 minutes post to the 5-MeO-DMT. In some preferred embodiments, the serotonin receptor modulator is quetiapine and the psychedelic is 5-MeO-DMT, wherein quetiapine is administered to post-treat between about 60 minutes and about 180 minutes post to the administration of 5-MeO-DMT.

In some embodiments, the serotonin receptor modulator is risperidone and the psychedelic is 5-MeO-DMT, wherein the risperidone is administered to post-treat at least 15 minutes post to the administration of 5-MeO-DMT. In some embodiments, the serotonin receptor modulator is risperidone and the psychedelic is 5-MeO-DMT, wherein the risperidone is administered to post-treat at least 30 minutes post to the 5-MeO-DMT. In some embodiments, the serotonin receptor modulator is risperidone and the psychedelic is 5-MeO-DMT, wherein the risperidone is administered to post-treat at least 60 minutes post to the 5-MeO-DMT. In some embodiments, the serotonin receptor modulator is risperidone and the psychedelic is 5-MeO-DMT, wherein the risperidone is administered to post-treat at least 90 minutes post to the 5-MeO-DMT. In some embodiments, the serotonin receptor modulator is risperidone and the psychedelic is 5-MeO-DMT, wherein the risperidone is administered to post-treat at least 120 minutes post to the 5-MeO-DMT. In some embodiments, the serotonin receptor modulator is risperidone and the psychedelic is 5-MeO-DMT, wherein the risperidone is administered to post-treat at least 150 minutes post to the 5-MeO-DMT. In some embodiments, the serotonin receptor modulator is risperidone and the psychedelic is 5-MeO-DMT, wherein the risperidone is administered to post-treat between about 15 minutes and about 150 minutes post to the 5-MeO-DMT. In some embodiments, the serotonin receptor modulator is risperidone and the psychedelic is 5-MeO-DMT, wherein the risperidone is administered to post-treat at least 180 minutes post to the 5-MeO-DMT. In some embodiments, the serotonin receptor modulator is risperidone and the psychedelic is 5-MeO-DMT, wherein the risperidone is administered to post-treat at least 210 minutes post to the 5-MeO-DMT. In some embodiments, the serotonin receptor modulator is risperidone and the psychedelic is 5-MeO-DMT, wherein the risperidone is administered to post-treat at least 240 minutes post to the 5-MeO-DMT. In some embodiments, the serotonin receptor modulator is risperidone and the psychedelic is 5-MeO-DMT, wherein the risperidone is administered to post-treat at least 270 minutes post to the 5-MeO-DMT. In some embodiments, the serotonin receptor modulator is risperidone and the psychedelic is 5-MeO-DMT, wherein the risperidone is administered to post-treat at least 300 minutes post to the 5-MeO-DMT. In some embodiments, the serotonin receptor modulator is risperidone and the psychedelic is 5-MeO-DMT, wherein the risperidone is administered to post-treat at least 330 minutes post to the 5-MeO-DMT. In some embodiments, the serotonin receptor modulator is risperidone and the psychedelic is 5-MeO-DMT, wherein the risperidone is administered to post-treat at least 360 minutes post to the 5-MeO-DMT. In some preferred embodiments, the serotonin receptor modulator is risperidone and the psychedelic is 5-MeO-DMT, wherein risperidone is administered to post-treat between about 60 minutes and about 180 minutes post to the administration of 5-MeO-DMT.

In some embodiments, the serotonin receptor modulator is eplivanserin and the psychedelic is 2C-B, wherein the eplivanserin is administered to post-treat at least 15 minutes post to the administration of 2C-B. In some embodiments, the serotonin receptor modulator is eplivanserin and the psychedelic is 2C-B, wherein the eplivanserin is administered to post-treat at least 30 minutes post to the 2C-B. In some embodiments, the serotonin receptor modulator is eplivanserin and the psychedelic is 2C-B, wherein the eplivanserin is administered to post-treat at least 60 minutes post to the 2C-B. In some embodiments, the serotonin receptor modulator is eplivanserin and the psychedelic is 2C-B, wherein the eplivanserin is administered to post-treat at least 90 minutes post to the 2C-B. In some embodiments, the serotonin receptor modulator is eplivanserin and the psychedelic is 2C-B, wherein the eplivanserin is administered to post-treat at least 120 minutes post to the 2C-B. In some embodiments, the serotonin receptor modulator is eplivanserin and the psychedelic is 2C-B, wherein the eplivanserin is administered to post-treat at least 150 minutes post to the 2C-B. In some embodiments, the serotonin receptor modulator is eplivanserin and the psychedelic is 2C-B, wherein the eplivanserin is administered to post-treat between about 15 minutes and about 150 minutes post to the 2C-B. In some embodiments, the serotonin receptor modulator is eplivanserin and the psychedelic is 2C-B, wherein the eplivanserin is administered to post-treat at least 180 minutes post to the 2C-B. In some embodiments, the serotonin receptor modulator is eplivanserin and the psychedelic is 2C-B, wherein the eplivanserin is administered to post-treat at least 210 minutes post to the 2C-B. In some embodiments, the serotonin receptor modulator is eplivanserin and the psychedelic is 2C-B, wherein the eplivanserin is administered to post-treat at least 240 minutes post to the 2C-B. In some embodiments, the serotonin receptor modulator is eplivanserin and the psychedelic is 2C-B, wherein the eplivanserin is administered to post-treat at least 270 minutes post to the 2C-B. In some embodiments, the serotonin receptor modulator is eplivanserin and the psychedelic is 2C-B, wherein the eplivanserin is administered to post-treat at least 300 minutes post to the 2C-B. In some embodiments, the serotonin receptor modulator is eplivanserin and the psychedelic is 2C-B, wherein the eplivanserin is administered to post-treat at least 330 minutes post to the 2C-B. In some embodiments, the serotonin receptor modulator is eplivanserin and the psychedelic is 2C-B, wherein the eplivanserin is administered to post-treat at least 360 minutes post to the 2C-B.

In some preferred embodiments, the serotonin receptor modulator is eplivanserin and the psychedelic is 2C-B, wherein eplivanserin is administered to post-treat between about 60 minutes and about 180 minutes post to the administration of 2C-B.

In some embodiments, the serotonin receptor modulator is volinanserin and the psychedelic is 2C-B, wherein the volinanserin is administered to post-treat at least 15 minutes post to the administration of 2C-B. In some embodiments, the serotonin receptor modulator is volinanserin and the psychedelic is 2C-B, wherein the volinanserin is administered to post-treat at least 30 minutes post to the 2C-B. In some embodiments, the serotonin receptor modulator is volinanserin and the psychedelic is 2C-B, wherein the volinanserin is administered to post-treat at least 60 minutes post to the 2C-B. In some embodiments, the serotonin receptor modulator is volinanserin and the psychedelic is 2C-B, wherein the volinanserin is administered to post-treat at least 90 minutes post to the 2C-B. In some embodiments, the serotonin receptor modulator is volinanserin and the psychedelic is 2C-B, wherein the volinanserin is administered to post-treat at least 120 minutes post to the 2C-B. In some embodiments, the serotonin receptor modulator is volinanserin and the psychedelic is 2C-B, wherein the volinanserin is administered to post-treat at least 150 minutes post to the 2C-B. In some embodiments, the serotonin receptor modulator is volinanserin and the psychedelic is 2C-B, wherein the volinanserin is administered to post-treat between about 15 minutes and about 150 minutes post to the 2C-B. In some embodiments, the serotonin receptor modulator is volinanserin and the psychedelic is 2C-B, wherein the volinanserin is administered to post-treat at least 180 minutes post to the 2C-B. In some embodiments, the serotonin receptor modulator is volinanserin and the psychedelic is 2C-B, wherein the volinanserin is administered to post-treat at least 210 minutes post to the 2C-B. In some embodiments, the serotonin receptor modulator is volinanserin and the psychedelic is 2C-B, wherein the volinanserin is administered to post-treat at least 240 minutes post to the 2C-B. In some embodiments, the serotonin receptor modulator is volinanserin and the psychedelic is 2C-B, wherein the volinanserin is administered to post-treat at least 270 minutes post to the 2C-B. In some embodiments, the serotonin receptor modulator is volinanserin and the psychedelic is 2C-B, wherein the volinanserin is administered to post-treat at least 300 minutes post to the 2C-B. In some embodiments, the serotonin receptor modulator is volinanserin and the psychedelic is 2C-B, wherein the volinanserin is administered to post-treat at least 330 minutes post to the 2C-B. In some embodiments, the serotonin receptor modulator is volinanserin and the psychedelic is 2C-B, wherein the volinanserin is administered to post-treat at least 360 minutes post to the 2C-B. In some preferred embodiments, the serotonin receptor modulator is volinanserin and the psychedelic is 2C-B, wherein volinanserin is administered to post-treat between about 60 minutes and about 180 minutes post to the administration of 2C-B.

In some embodiments, the serotonin receptor modulator is ketanserin and the psychedelic is 2C-B, wherein the ketanserin is administered to post-treat at least 15 minutes post to the administration of 2C-B. In some embodiments, the serotonin receptor modulator is ketanserin and the psychedelic is 2C-B, wherein the ketanserin is administered to post-treat at least 30 minutes post to the 2C-B. In some embodiments, the serotonin receptor modulator is ketanserin and the psychedelic is 2C-B, wherein the ketanserin is administered to post-treat at least 60 minutes post to the 2C-B. In some embodiments, the serotonin receptor modulator is ketanserin and the psychedelic is 2C-B, wherein the ketanserin is administered to post-treat at least 90 minutes post to the 2C-B. In some embodiments, the serotonin receptor modulator is ketanserin and the psychedelic is 2C-B, wherein the ketanserin is administered to post-treat at least 120 minutes post to the 2C-B. In some embodiments, the serotonin receptor modulator is ketanserin and the psychedelic is 2C-B, wherein the ketanserin is administered to post-treat at least 150 minutes post to the 2C-B. In some embodiments, the serotonin receptor modulator is ketanserin and the psychedelic is 2C-B, wherein the ketanserin is administered to post-treat between about 15 minutes and about 150 minutes post to the 2C-B. In some embodiments, the serotonin receptor modulator is ketanserin and the psychedelic is 2C-B, wherein the ketanserin is administered to post-treat at least 180 minutes post to the 2C-B. In some embodiments, the serotonin receptor modulator is ketanserin and the psychedelic is 2C-B, wherein the ketanserin is administered to post-treat at least 210 minutes post to the 2C-B. In some embodiments, the serotonin receptor modulator is ketanserin and the psychedelic is 2C-B, wherein the ketanserin is administered to post-treat at least 240 minutes post to the 2C-B. In some embodiments, the serotonin receptor modulator is ketanserin and the psychedelic is 2C-B, wherein the ketanserin is administered to post-treat at least 270 minutes post to the 2C-B. In some embodiments, the serotonin receptor modulator is ketanserin and the psychedelic is 2C-B, wherein the ketanserin is administered to post-treat at least 300 minutes post to the 2C-B. In some embodiments, the serotonin receptor modulator is ketanserin and the psychedelic is 2C-B, wherein the ketanserin is administered to post-treat at least 330 minutes post to the 2C-B. In some embodiments, the serotonin receptor modulator is ketanserin and the psychedelic is 2C-B, wherein the ketanserin is administered to post-treat at least 360 minutes post to the 2C-B. In some preferred embodiments, the serotonin receptor modulator is ketanserin and the psychedelic is 2C-B, wherein ketanserin is administered to post-treat between about 60 minutes and about 180 minutes post to the administration of 2C-B.

In some embodiments, the serotonin receptor modulator is ritanserin and the psychedelic is 2C-B, wherein the ritanserin is administered to post-treat at least 15 minutes post to the 2C-B. In some embodiments, the serotonin receptor modulator is ritanserin and the psychedelic is 2C-B, wherein the ritanserin is administered to post-treat at least 30 minutes post to the 2C-B. In some embodiments, the serotonin receptor modulator is ritanserin and the psychedelic is 2C-B, wherein the ritanserin is administered to post-treat at least 60 minutes post to the 2C-B. In some embodiments, the serotonin receptor modulator is ritanserin and the psychedelic is 2C-B, wherein the ritanserin is administered to post-treat at least 90 minutes post to the 2C-B. In some embodiments, the serotonin receptor modulator is ritanserin and the psychedelic is 2C-B, wherein the ritanserin is administered to post-treat at least 120 minutes post to the 2C-B. In some embodiments, the serotonin receptor modulator is ritanserin and the psychedelic is 2C-B, wherein the ritanserin is administered to post-treat at least 150 minutes post to the 2C-B. In some embodiments, the serotonin receptor modulator is ritanserin and the psychedelic is 2C-B, wherein the ritanserin is administered to post-treat between about 15 minutes and about 150 minutes post to the 2C-B. In some embodiments, the serotonin receptor modulator is ritanserin and the psychedelic is 2C-B, wherein the ritanserin is administered to post-treat at least 180 minutes post to the 2C-B. In some embodiments, the serotonin receptor modulator is ritanserin and the psychedelic is 2C-B, wherein the ritanserin is administered to post-treat at least 210 minutes post to the 2C-B. In some embodiments, the serotonin receptor modulator is ritanserin and the psychedelic is 2C-B, wherein the ritanserin is administered to post-treat at least 240 minutes post to the 2C-B. In some embodiments, the serotonin receptor modulator is ritanserin and the psychedelic is 2C-B, wherein the ritanserin is administered to post-treat at least 270 minutes post to the 2C-B. In some embodiments, the serotonin receptor modulator is ritanserin and the psychedelic is 2C-B, wherein the ritanserin is administered to post-treat at least 300 minutes post to the 2C-B. In some embodiments, the serotonin receptor modulator is ritanserin and the psychedelic is 2C-B, wherein the ritanserin is administered to post-treat at least 330 minutes post to the 2C-B. In some embodiments, the serotonin receptor modulator is ritanserin and the psychedelic is 2C-B, wherein the ritanserin is administered to post-treat at least 360 minutes post to the 2C-B. In some preferred embodiments, the serotonin receptor modulator is ritanserin and the psychedelic is 2C-B, wherein ritanserin is administered to post-treat between about 60 minutes and about 180 minutes post to the administration of 2C-B.

In some embodiments, the serotonin receptor modulator is pimavanserin and the psychedelic is 2C-B, wherein the pimavanserin is administered to post-treat at least 15 minutes post to the 2C-B. In some embodiments, the serotonin receptor modulator is pimavanserin and the psychedelic is 2C-B, wherein the pimavanserin is administered to post-treat at least 30 minutes post to the 2C-B. In some embodiments, the serotonin receptor modulator is pimavanserin and the psychedelic is 2C-B, wherein the pimavanserin is administered to post-treat at least 60 minutes post to the 2C-B. In some embodiments, the serotonin receptor modulator is pimavanserin and the psychedelic is 2C-B, wherein the pimavanserin is administered to post-treat at least 90 minutes post to the 2C-B. In some embodiments, the serotonin receptor modulator is pimavanserin and the psychedelic is 2C-B, wherein the pimavanserin is administered to post-treat at least 120 minutes post to the 2C-B. In some embodiments, the serotonin receptor modulator is pimavanserin and the psychedelic is 2C-B, wherein the pimavanserin is administered to post-treat at least 150 minutes post to the 2C-B. In some embodiments, the serotonin receptor modulator is pimavanserin and the psychedelic is 2C-B, wherein the pimavanserin is administered to post-treat between about 15 minutes and about 150 minutes post to the 2C-B. In some embodiments, the serotonin receptor modulator is pimavanserin and the psychedelic is 2C-B, wherein the pimavanserin is administered to post-treat at least 180 minutes post to the 2C-B. In some embodiments, the serotonin receptor modulator is pimavanserin and the psychedelic is 2C-B, wherein the pimavanserin is administered to post-treat at least 210 minutes post to the 2C-B. In some embodiments, the serotonin receptor modulator is pimavanserin and the psychedelic is 2C-B, wherein the pimavanserin is administered to post-treat at least 240 minutes post to the 2C-B. In some embodiments, the serotonin receptor modulator is pimavanserin and the psychedelic is 2C-B, wherein the pimavanserin is administered to post-treat at least 270 minutes post to the 2C-B. In some embodiments, the serotonin receptor modulator is pimavanserin and the psychedelic is 2C-B, wherein the pimavanserin is administered to post-treat at least 300 minutes post to the 2C-B. In some embodiments, the serotonin receptor modulator is pimavanserin and the psychedelic is 2C-B, wherein the pimavanserin is administered to post-treat at least 330 minutes post to the 2C-B. In some embodiments, the serotonin receptor modulator is pimavanserin and the psychedelic is 2C-B, wherein the pimavanserin is administered to post-treat at least 360 minutes post to the 2C-B. In some preferred embodiments, the serotonin receptor modulator is pimavanserin and the psychedelic is 2C-B, wherein pimavanserin is administered to post-treat between about 60 minutes and about 180 minutes post to the administration of 2C-B.

In some embodiments, the serotonin receptor modulator is nelotanserin and the psychedelic is 2C-B, wherein the nelotanserin is administered to post-treat at least 15 minutes post to the 2C-B. In some embodiments, the serotonin receptor modulator is nelotanserin and the psychedelic is 2C-B, wherein the nelotanserin is administered to post-treat at least 30 minutes post to the 2C-B. In some embodiments, the serotonin receptor modulator is nelotanserin and the psychedelic is 2C-B, wherein the nelotanserin is administered to post-treat at least 60 minutes post to the 2C-B. In some embodiments, the serotonin receptor modulator is nelotanserin and the psychedelic is 2C-B, wherein the nelotanserin is administered to post-treat at least 90 minutes post to the 2C-B. In some embodiments, the serotonin receptor modulator is nelotanserin and the psychedelic is 2C-B, wherein the nelotanserin is administered to post-treat at least 120 minutes post to the 2C-B. In some embodiments, the serotonin receptor modulator is nelotanserin and the psychedelic is 2C-B, wherein the nelotanserin is administered to post-treat at least 150 minutes post to the 2C-B. In some embodiments, the serotonin receptor modulator is nelotanserin and the psychedelic is 2C-B, wherein the nelotanserin is administered to post-treat between about 15 minutes and about 150 minutes post to the 2C-B. In some embodiments, the serotonin receptor modulator is nelotanserin and the psychedelic is 2C-B, wherein the nelotanserin is administered to post-treat at least 180 minutes post to the 2C-B. In some embodiments, the serotonin receptor modulator is nelotanserin and the psychedelic is 2C-B, wherein the nelotanserin is administered to post-treat at least 210 minutes post to the 2C-B. In some embodiments, the serotonin receptor modulator is nelotanserin and the psychedelic is 2C-B, wherein the nelotanserin is administered to post-treat at least 240 minutes post to the 2C-B. In some embodiments, the serotonin receptor modulator is nelotanserin and the psychedelic is 2C-B, wherein the nelotanserin is administered to post-treat at least 270 minutes post to the 2C-B. In some embodiments, the serotonin receptor modulator is nelotanserin and the psychedelic is 2C-B, wherein the nelotanserin is administered to post-treat at least 300 minutes post to the 2C-B. In some embodiments, the serotonin receptor modulator is nelotanserin and the psychedelic is 2C-B, wherein the nelotanserin is administered to post-treat at least 330 minutes post to the 2C-B. In some embodiments, the serotonin receptor modulator is nelotanserin and the psychedelic is 2C-B, wherein the nelotanserin is administered to post-treat at least 360 minutes post to the 2C-B. In some preferred embodiments, the serotonin receptor modulator is nelotanserin and the psychedelic is 2C-B, wherein nelotanserin is administered to post-treat between about 60 minutes and about 180 minutes post to the administration of 2C-B.

In some embodiments, the serotonin receptor modulator is pruvanserin and the psychedelic is 2C-B, wherein the pruvanserin is administered to post-treat at least 15 minutes post to the 2C-B. In some embodiments, the serotonin receptor modulator is pruvanserin and the psychedelic is 2C-B, wherein the pruvanserin is administered to post-treat at least 30 minutes post to the 2C-B. In some embodiments, the serotonin receptor modulator is pruvanserin and the psychedelic is 2C-B, wherein the pruvanserin is administered to post-treat at least 60 minutes post to the 2C-B. In some embodiments, the serotonin receptor modulator is pruvanserin and the psychedelic is 2C-B, wherein the pruvanserin is administered to post-treat at least 90 minutes post to the 2C-B. In some embodiments, the serotonin receptor modulator is pruvanserin and the psychedelic is 2C-B, wherein the pruvanserin is administered to post-treat at least 120 minutes post to the 2C-B. In some embodiments, the serotonin receptor modulator is pruvanserin and the psychedelic is 2C-B, wherein the pruvanserin is administered to post-treat at least 150 minutes post to the 2C-B. In some embodiments, the serotonin receptor modulator is pruvanserin and the psychedelic is 2C-B, wherein the pruvanserin is administered to post-treat between about 15 minutes and about 150 minutes post to the 2C-B. In some embodiments, the serotonin receptor modulator is pruvanserin and the psychedelic is 2C-B, wherein the pruvanserin is administered to post-treat at least 180 minutes post to the 2C-B. In some embodiments, the serotonin receptor modulator is pruvanserin and the psychedelic is 2C-B, wherein the pruvanserin is administered to post-treat at least 210 minutes post to the 2C-B. In some embodiments, the serotonin receptor modulator is pruvanserin and the psychedelic is 2C-B, wherein the pruvanserin is administered to post-treat at least 240 minutes post to the 2C-B. In some embodiments, the serotonin receptor modulator is pruvanserin and the psychedelic is 2C-B, wherein the pruvanserin is administered to post-treat at least 270 minutes post to the 2C-B. In some embodiments, the serotonin receptor modulator is pruvanserin and the psychedelic is 2C-B, wherein the pruvanserin is administered to post-treat at least 300 minutes post to the 2C-B. In some embodiments, the serotonin receptor modulator is pruvanserin and the psychedelic is 2C-B, wherein the pruvanserin is administered to post-treat at least 330 minutes post to the 2C-B. In some embodiments, the serotonin receptor modulator is pruvanserin and the psychedelic is 2C-B, wherein the pruvanserin is administered to post-treat at least 360 minutes post to the 2C-B. In some preferred embodiments, the serotonin receptor modulator is pruvanserin and the psychedelic is 2C-B, wherein pruvanserin is administered to post-treat between about 60 minutes and about 180 minutes post to the administration of 2C-B.

In some embodiments, the serotonin receptor modulator is flibanserin and the psychedelic is 2C-B, wherein the flibanserin is administered to post-treat at least 15 minutes post to the administration of 2C-B. In some embodiments, the serotonin receptor modulator is flibanserin and the psychedelic is 2C-B, wherein the flibanserin is administered to post-treat at least 30 minutes post to the 2C-B. In some embodiments, the serotonin receptor modulator is flibanserin and the psychedelic is 2C-B, wherein the flibanserin is administered to post-treat at least 60 minutes post to the 2C-B. In some embodiments, the serotonin receptor modulator is flibanserin and the psychedelic is 2C-B, wherein the flibanserin is administered to post-treat at least 90 minutes post to the 2C-B. In some embodiments, the serotonin receptor modulator is flibanserin and the psychedelic is 2C-B, wherein the flibanserin is administered to post-treat at least 120 minutes post to the 2C-B. In some embodiments, the serotonin receptor modulator is flibanserin and the psychedelic is 2C-B, wherein the flibanserin is administered to post-treat at least 150 minutes post to the 2C-B. In some embodiments, the serotonin receptor modulator is flibanserin and the psychedelic is 2C-B, wherein the flibanserin is administered to post-treat between about 15 minutes and about 150 minutes post to the 2C-B. In some embodiments, the serotonin receptor modulator is flibanserin and the psychedelic is 2C-B, wherein the flibanserin is administered to post-treat at least 180 minutes post to the 2C-B. In some embodiments, the serotonin receptor modulator is flibanserin and the psychedelic is 2C-B, wherein the flibanserin is administered to post-treat at least 210 minutes post to the 2C-B. In some embodiments, the serotonin receptor modulator is flibanserin and the psychedelic is 2C-B, wherein the flibanserin is administered to post-treat at least 240 minutes post to the 2C-B. In some embodiments, the serotonin receptor modulator is flibanserin and the psychedelic is 2C-B, wherein the flibanserin is administered to post-treat at least 270 minutes post to the 2C-B. In some embodiments, the serotonin receptor modulator is flibanserin and the psychedelic is 2C-B, wherein the flibanserin is administered to post-treat at least 300 minutes post to the 2C-B. In some embodiments, the serotonin receptor modulator is flibanserin and the psychedelic is 2C-B, wherein the flibanserin is administered to post-treat at least 330 minutes post to the 2C-B. In some embodiments, the serotonin receptor modulator is flibanserin and the psychedelic is 2C-B, wherein the flibanserin is administered to post-treat at least 360 minutes post to the 2C-B. In some preferred embodiments, the serotonin receptor modulator is flibanserin and the psychedelic is 2C-B, wherein flibanserin is administered to post-treat between about 60 minutes and about 180 minutes post to the administration of 2C-B.

In some embodiments, the serotonin receptor modulator is olanzapine and the psychedelic is 2C-B, wherein the olanzapine is administered to post-treat at least 15 minutes post to the administration of 2C-B. In some embodiments, the serotonin receptor modulator is olanzapine and the psychedelic is 2C-B, wherein the olanzapine is administered to post-treat at least 30 minutes post to the 2C-B. In some embodiments, the serotonin receptor modulator is olanzapine and the psychedelic is 2C-B, wherein the olanzapine is administered to post-treat at least 60 minutes post to the 2C-B. In some embodiments, the serotonin receptor modulator is olanzapine and the psychedelic is 2C-B, wherein the olanzapine is administered to post-treat at least 90 minutes post to the 2C-B. In some embodiments, the serotonin receptor modulator is olanzapine and the psychedelic is 2C-B, wherein the olanzapine is administered to post-treat at least 120 minutes post to the 2C-B. In some embodiments, the serotonin receptor modulator is olanzapine and the psychedelic is 2C-B, wherein the olanzapine is administered to post-treat at least 150 minutes post to the 2C-B. In some embodiments, the serotonin receptor modulator is olanzapine and the psychedelic is 2C-B, wherein the olanzapine is administered to post-treat between about 15 minutes and about 150 minutes post to the 2C-B. In some embodiments, the serotonin receptor modulator is olanzapine and the psychedelic is 2C-B, wherein the olanzapine is administered to post-treat at least 180 minutes post to the 2C-B. In some embodiments, the serotonin receptor modulator is olanzapine and the psychedelic is 2C-B, wherein the olanzapine is administered to post-treat at least 210 minutes post to the 2C-B. In some embodiments, the serotonin receptor modulator is olanzapine and the psychedelic is 2C-B, wherein the olanzapine is administered to post-treat at least 240 minutes post to the 2C-B. In some embodiments, the serotonin receptor modulator is olanzapine and the psychedelic is 2C-B, wherein the olanzapine is administered to post-treat at least 270 minutes post to the 2C-B. In some embodiments, the serotonin receptor modulator is olanzapine and the psychedelic is 2C-B, wherein the olanzapine is administered to post-treat at least 300 minutes post to the 2C-B. In some embodiments, the serotonin receptor modulator is olanzapine and the psychedelic is 2C-B, wherein the olanzapine is administered to post-treat at least 330 minutes post to the 2C-B. In some embodiments, the serotonin receptor modulator is olanzapine and the psychedelic is 2C-B, wherein the olanzapine is administered to post-treat at least 360 minutes post to the 2C-B. In some preferred embodiments, the serotonin receptor modulator is olanzapine and the psychedelic is 2C-B, wherein olanzapine is administered to post-treat between about 60 minutes and about 180 minutes post to the administration of 2C-B.

In some embodiments, the serotonin receptor modulator is quetiapine and the psychedelic is 2C-B, wherein the quetiapine is administered to post-treat at least 15 minutes post to the administration of 2C-B. In some embodiments, the serotonin receptor modulator is quetiapine and the psychedelic is 2C-B, wherein the quetiapine is administered to post-treat at least 30 minutes post to the 2C-B. In some embodiments, the serotonin receptor modulator is quetiapine and the psychedelic is 2C-B, wherein the quetiapine is administered to post-treat at least 60 minutes post to the 2C-B. In some embodiments, the serotonin receptor modulator is quetiapine and the psychedelic is 2C-B, wherein the quetiapine is administered to post-treat at least 90 minutes post to the 2C-B. In some embodiments, the serotonin receptor modulator is quetiapine and the psychedelic is 2C-B, wherein the quetiapine is administered to post-treat at least 120 minutes post to the 2C-B. In some embodiments, the serotonin receptor modulator is quetiapine and the psychedelic is 2C-B, wherein the quetiapine is administered to post-treat at least 150 minutes post to the 2C-B. In some embodiments, the serotonin receptor modulator is quetiapine and the psychedelic is 2C-B, wherein the quetiapine is administered to post-treat between about 15 minutes and about 150 minutes post to the 2C-B. In some embodiments, the serotonin receptor modulator is quetiapine and the psychedelic is 2C-B, wherein the quetiapine is administered to post-treat at least 180 minutes post to the 2C-B. In some embodiments, the serotonin receptor modulator is quetiapine and the psychedelic is 2C-B, wherein the quetiapine is administered to post-treat at least 210 minutes post to the 2C-B. In some embodiments, the serotonin receptor modulator is quetiapine and the psychedelic is 2C-B, wherein the quetiapine is administered to post-treat at least 240 minutes post to the 2C-B. In some embodiments, the serotonin receptor modulator is quetiapine and the psychedelic is 2C-B, wherein the quetiapine is administered to post-treat at least 270 minutes post to the 2C-B. In some embodiments, the serotonin receptor modulator is quetiapine and the psychedelic is 2C-B, wherein the quetiapine is administered to post-treat at least 300 minutes post to the 2C-B. In some embodiments, the serotonin receptor modulator is quetiapine and the psychedelic is 2C-B, wherein the quetiapine is administered to post-treat at least 330 minutes post to the 2C-B. In some embodiments, the serotonin receptor modulator is quetiapine and the psychedelic is 2C-B, wherein the quetiapine is administered to post-treat at least 360 minutes post to the 2C-B. In some preferred embodiments, the serotonin receptor modulator is quetiapine and the psychedelic is 2C-B, wherein quetiapine is administered to post-treat between about 60 minutes and about 180 minutes post to the administration of 2C-B.

In some embodiments, the serotonin receptor modulator is risperidone and the psychedelic is 2C-B, wherein the risperidone is administered to post-treat at least 15 minutes post to the administration of 2C-B. In some embodiments, the serotonin receptor modulator is risperidone and the psychedelic is 2C-B, wherein the risperidone is administered to post-treat at least 30 minutes post to the 2C-B. In some embodiments, the serotonin receptor modulator is risperidone and the psychedelic is 2C-B, wherein the risperidone is administered to post-treat at least 60 minutes post to the 2C-B. In some embodiments, the serotonin receptor modulator is risperidone and the psychedelic is 2C-B, wherein the risperidone is administered to post-treat at least 90 minutes post to the 2C-B. In some embodiments, the serotonin receptor modulator is risperidone and the psychedelic is 2C-B, wherein the risperidone is administered to post-treat at least 120 minutes post to the 2C-B. In some embodiments, the serotonin receptor modulator is risperidone and the psychedelic is 2C-B, wherein the risperidone is administered to post-treat at least 150 minutes post to the 2C-B. In some embodiments, the serotonin receptor modulator is risperidone and the psychedelic is 2C-B, wherein the risperidone is administered to post-treat between about 15 minutes and about 150 minutes post to the 2C-B. In some embodiments, the serotonin receptor modulator is risperidone and the psychedelic is 2C-B, wherein the risperidone is administered to post-treat at least 180 minutes post to the 2C-B. In some embodiments, the serotonin receptor modulator is risperidone and the psychedelic is 2C-B, wherein the risperidone is administered to post-treat at least 210 minutes post to the 2C-B. In some embodiments, the serotonin receptor modulator is risperidone and the psychedelic is 2C-B, wherein the risperidone is administered to post-treat at least 240 minutes post to the 2C-B. In some embodiments, the serotonin receptor modulator is risperidone and the psychedelic is 2C-B, wherein the risperidone is administered to post-treat at least 270 minutes post to the 2C-B. In some embodiments, the serotonin receptor modulator is risperidone and the psychedelic is 2C-B, wherein the risperidone is administered to post-treat at least 300 minutes post to the 2C-B. In some embodiments, the serotonin receptor modulator is risperidone and the psychedelic is 2C-B, wherein the risperidone is administered to post-treat at least 330 minutes post to the 2C-B. In some embodiments, the serotonin receptor modulator is risperidone and the psychedelic is 2C-B, wherein the risperidone is administered to post-treat at least 360 minutes post to the 2C-B. In some preferred embodiments, the serotonin receptor modulator is risperidone and the psychedelic is 2C-B, wherein risperidone is administered to post-treat between about 60 minutes and about 180 minutes post to the administration of 2C-B.

In some embodiments, the serotonin receptor modulator is eplivanserin and the psychedelic is ibogaine, wherein the eplivanserin is administered to post-treat at least 15 minutes post to the administration of ibogaine. In some embodiments, the serotonin receptor modulator is eplivanserin and the psychedelic is ibogaine, wherein the eplivanserin is administered to post-treat at least 30 minutes post to the ibogaine. In some embodiments, the serotonin receptor modulator is eplivanserin and the psychedelic is ibogaine, wherein the eplivanserin is administered to post-treat at least 60 minutes post to the ibogaine. In some embodiments, the serotonin receptor modulator is eplivanserin and the psychedelic is ibogaine, wherein the eplivanserin is administered to post-treat at least 90 minutes post to the ibogaine. In some embodiments, the serotonin receptor modulator is eplivanserin and the psychedelic is ibogaine, wherein the eplivanserin is administered to post-treat at least 120 minutes post to the ibogaine. In some embodiments, the serotonin receptor modulator is eplivanserin and the psychedelic is ibogaine, wherein the eplivanserin is administered to post-treat at least 150 minutes post to the ibogaine. In some embodiments, the serotonin receptor modulator is eplivanserin and the psychedelic is ibogaine, wherein the eplivanserin is administered to post-treat between about 15 minutes and about 150 minutes post to the ibogaine. In some embodiments, the serotonin receptor modulator is eplivanserin and the psychedelic is ibogaine, wherein the eplivanserin is administered to post-treat at least 180 minutes post to the ibogaine. In some embodiments, the serotonin receptor modulator is eplivanserin and the psychedelic is ibogaine, wherein the eplivanserin is administered to post-treat at least 210 minutes post to the ibogaine. In some embodiments, the serotonin receptor modulator is eplivanserin and the psychedelic is ibogaine, wherein the eplivanserin is administered to post-treat at least 240 minutes post to the ibogaine. In some embodiments, the serotonin receptor modulator is eplivanserin and the psychedelic is ibogaine, wherein the eplivanserin is administered to post-treat at least 270 minutes post to the ibogaine. In some embodiments, the serotonin receptor modulator is eplivanserin and the psychedelic is ibogaine, wherein the eplivanserin is administered to post-treat at least 300 minutes post to the ibogaine. In some embodiments, the serotonin receptor modulator is eplivanserin and the psychedelic is ibogaine, wherein the eplivanserin is administered to post-treat at least 330 minutes post to the ibogaine. In some embodiments, the serotonin receptor modulator is eplivanserin and the psychedelic is ibogaine, wherein the eplivanserin is administered to post-treat at least 360 minutes post to the ibogaine.

In some preferred embodiments, the serotonin receptor modulator is eplivanserin and the psychedelic is ibogaine, wherein eplivanserin is administered to post-treat between about 60 minutes and about 180 minutes post to the administration of ibogaine.

In some embodiments, the serotonin receptor modulator is volinanserin and the psychedelic is ibogaine, wherein the volinanserin is administered to post-treat at least 15 minutes post to the administration of ibogaine. In some embodiments, the serotonin receptor modulator is volinanserin and the psychedelic is ibogaine, wherein the volinanserin is administered to post-treat at least 30 minutes post to the ibogaine. In some embodiments, the serotonin receptor modulator is volinanserin and the psychedelic is ibogaine, wherein the volinanserin is administered to post-treat at least 60 minutes post to the ibogaine. In some embodiments, the serotonin receptor modulator is volinanserin and the psychedelic is ibogaine, wherein the volinanserin is administered to post-treat at least 90 minutes post to the ibogaine. In some embodiments, the serotonin receptor modulator is volinanserin and the psychedelic is ibogaine, wherein the volinanserin is administered to post-treat at least 120 minutes post to the ibogaine. In some embodiments, the serotonin receptor modulator is volinanserin and the psychedelic is ibogaine, wherein the volinanserin is administered to post-treat at least 150 minutes post to the ibogaine. In some embodiments, the serotonin receptor modulator is volinanserin and the psychedelic is ibogaine, wherein the volinanserin is administered to post-treat between about 15 minutes and about 150 minutes post to the ibogaine. In some embodiments, the serotonin receptor modulator is volinanserin and the psychedelic is ibogaine, wherein the volinanserin is administered to post-treat at least 180 minutes post to the ibogaine. In some embodiments, the serotonin receptor modulator is volinanserin and the psychedelic is ibogaine, wherein the volinanserin is administered to post-treat at least 210 minutes post to the ibogaine. In some embodiments, the serotonin receptor modulator is volinanserin and the psychedelic is ibogaine, wherein the volinanserin is administered to post-treat at least 240 minutes post to the ibogaine. In some embodiments, the serotonin receptor modulator is volinanserin and the psychedelic is ibogaine, wherein the volinanserin is administered to post-treat at least 270 minutes post to the ibogaine. In some embodiments, the serotonin receptor modulator is volinanserin and the psychedelic is ibogaine, wherein the volinanserin is administered to post-treat at least 300 minutes post to the ibogaine. In some embodiments, the serotonin receptor modulator is volinanserin and the psychedelic is ibogaine, wherein the volinanserin is administered to post-treat at least 330 minutes post to the ibogaine. In some embodiments, the serotonin receptor modulator is volinanserin and the psychedelic is ibogaine, wherein the volinanserin is administered to post-treat at least 360 minutes post to the ibogaine. In some preferred embodiments, the serotonin receptor modulator is volinanserin and the psychedelic is ibogaine, wherein volinanserin is administered to post-treat between about 60 minutes and about 180 minutes post to the administration of ibogaine.

In some embodiments, the serotonin receptor modulator is ketanserin and the psychedelic is ibogaine, wherein the ketanserin is administered to post-treat at least 15 minutes post to the administration of ibogaine. In some embodiments, the serotonin receptor modulator is ketanserin and the psychedelic is ibogaine, wherein the ketanserin is administered to post-treat at least 30 minutes post to the ibogaine. In some embodiments, the serotonin receptor modulator is ketanserin and the psychedelic is ibogaine, wherein the ketanserin is administered to post-treat at least 60 minutes post to the ibogaine. In some embodiments, the serotonin receptor modulator is ketanserin and the psychedelic is ibogaine, wherein the ketanserin is administered to post-treat at least 90 minutes post to the ibogaine. In some embodiments, the serotonin receptor modulator is ketanserin and the psychedelic is ibogaine, wherein the ketanserin is administered to post-treat at least 120 minutes post to the ibogaine. In some embodiments, the serotonin receptor modulator is ketanserin and the psychedelic is ibogaine, wherein the ketanserin is administered to post-treat at least 150 minutes post to the ibogaine. In some embodiments, the serotonin receptor modulator is ketanserin and the psychedelic is ibogaine, wherein the ketanserin is administered to post-treat between about 15 minutes and about 150 minutes post to the ibogaine. In some embodiments, the serotonin receptor modulator is ketanserin and the psychedelic is ibogaine, wherein the ketanserin is administered to post-treat at least 180 minutes post to the ibogaine. In some embodiments, the serotonin receptor modulator is ketanserin and the psychedelic is ibogaine, wherein the ketanserin is administered to post-treat at least 210 minutes post to the ibogaine. In some embodiments, the serotonin receptor modulator is ketanserin and the psychedelic is ibogaine, wherein the ketanserin is administered to post-treat at least 240 minutes post to the ibogaine. In some embodiments, the serotonin receptor modulator is ketanserin and the psychedelic is ibogaine, wherein the ketanserin is administered to post-treat at least 270 minutes post to the ibogaine. In some embodiments, the serotonin receptor modulator is ketanserin and the psychedelic is ibogaine, wherein the ketanserin is administered to post-treat at least 300 minutes post to the ibogaine. In some embodiments, the serotonin receptor modulator is ketanserin and the psychedelic is ibogaine, wherein the ketanserin is administered to post-treat at least 330 minutes post to the ibogaine. In some embodiments, the serotonin receptor modulator is ketanserin and the psychedelic is ibogaine, wherein the ketanserin is administered to post-treat at least 360 minutes post to the ibogaine. In some preferred embodiments, the serotonin receptor modulator is ketanserin and the psychedelic is ibogaine, wherein ketanserin is administered to post-treat between about 60 minutes and about 180 minutes post to the administration of ibogaine.

In some embodiments, the serotonin receptor modulator is ritanserin and the psychedelic is ibogaine, wherein the ritanserin is administered to post-treat at least 15 minutes post to the ibogaine. In some embodiments, the serotonin receptor modulator is ritanserin and the psychedelic is ibogaine, wherein the ritanserin is administered to post-treat at least 30 minutes post to the ibogaine. In some embodiments, the serotonin receptor modulator is ritanserin and the psychedelic is ibogaine, wherein the ritanserin is administered to post-treat at least 60 minutes post to the ibogaine. In some embodiments, the serotonin receptor modulator is ritanserin and the psychedelic is ibogaine, wherein the ritanserin is administered to post-treat at least 90 minutes post to the ibogaine. In some embodiments, the serotonin receptor modulator is ritanserin and the psychedelic is ibogaine, wherein the ritanserin is administered to post-treat at least 120 minutes post to the ibogaine. In some embodiments, the serotonin receptor modulator is ritanserin and the psychedelic is ibogaine, wherein the ritanserin is administered to post-treat at least 150 minutes post to the ibogaine. In some embodiments, the serotonin receptor modulator is ritanserin and the psychedelic is ibogaine, wherein the ritanserin is administered to post-treat between about 15 minutes and about 150 minutes post to the ibogaine. In some embodiments, the serotonin receptor modulator is ritanserin and the psychedelic is ibogaine, wherein the ritanserin is administered to post-treat at least 180 minutes post to the ibogaine. In some embodiments, the serotonin receptor modulator is ritanserin and the psychedelic is ibogaine, wherein the ritanserin is administered to post-treat at least 210 minutes post to the ibogaine. In some embodiments, the serotonin receptor modulator is ritanserin and the psychedelic is ibogaine, wherein the ritanserin is administered to post-treat at least 240 minutes post to the ibogaine. In some embodiments, the serotonin receptor modulator is ritanserin and the psychedelic is ibogaine, wherein the ritanserin is administered to post-treat at least 270 minutes post to the ibogaine. In some embodiments, the serotonin receptor modulator is ritanserin and the psychedelic is ibogaine, wherein the ritanserin is administered to post-treat at least 300 minutes post to the ibogaine. In some embodiments, the serotonin receptor modulator is ritanserin and the psychedelic is ibogaine, wherein the ritanserin is administered to post-treat at least 330 minutes post to the ibogaine. In some embodiments, the serotonin receptor modulator is ritanserin and the psychedelic is ibogaine, wherein the ritanserin is administered to post-treat at least 360 minutes post to the ibogaine. In some preferred embodiments, the serotonin receptor modulator is ritanserin and the psychedelic is ibogaine, wherein ritanserin is administered to post-treat between about 60 minutes and about 180 minutes post to the administration of ibogaine.

In some embodiments, the serotonin receptor modulator is pimavanserin and the psychedelic is ibogaine, wherein the pimavanserin is administered to post-treat at least 15 minutes post to the ibogaine. In some embodiments, the serotonin receptor modulator is pimavanserin and the psychedelic is ibogaine, wherein the pimavanserin is administered to post-treat at least 30 minutes post to the ibogaine. In some embodiments, the serotonin receptor modulator is pimavanserin and the psychedelic is ibogaine, wherein the pimavanserin is administered to post-treat at least 60 minutes post to the ibogaine. In some embodiments, the serotonin receptor modulator is pimavanserin and the psychedelic is ibogaine, wherein the pimavanserin is administered to post-treat at least 90 minutes post to the ibogaine. In some embodiments, the serotonin receptor modulator is pimavanserin and the psychedelic is ibogaine, wherein the pimavanserin is administered to post-treat at least 120 minutes post to the ibogaine. In some embodiments, the serotonin receptor modulator is pimavanserin and the psychedelic is ibogaine, wherein the pimavanserin is administered to post-treat at least 150 minutes post to the ibogaine. In some embodiments, the serotonin receptor modulator is pimavanserin and the psychedelic is ibogaine, wherein the pimavanserin is administered to post-treat between about 15 minutes and about 150 minutes post to the ibogaine. In some embodiments, the serotonin receptor modulator is pimavanserin and the psychedelic is ibogaine, wherein the pimavanserin is administered to post-treat at least 180 minutes post to the ibogaine. In some embodiments, the serotonin receptor modulator is pimavanserin and the psychedelic is ibogaine, wherein the pimavanserin is administered to post-treat at least 210 minutes post to the ibogaine. In some embodiments, the serotonin receptor modulator is pimavanserin and the psychedelic is ibogaine, wherein the pimavanserin is administered to post-treat at least 240 minutes post to the ibogaine. In some embodiments, the serotonin receptor modulator is pimavanserin and the psychedelic is ibogaine, wherein the pimavanserin is administered to post-treat at least 270 minutes post to the ibogaine. In some embodiments, the serotonin receptor modulator is pimavanserin and the psychedelic is ibogaine, wherein the pimavanserin is administered to post-treat at least 300 minutes post to the ibogaine. In some embodiments, the serotonin receptor modulator is pimavanserin and the psychedelic is ibogaine, wherein the pimavanserin is administered to post-treat at least 330 minutes post to the ibogaine. In some embodiments, the serotonin receptor modulator is pimavanserin and the psychedelic is ibogaine, wherein the pimavanserin is administered to post-treat at least 360 minutes post to the ibogaine. In some preferred embodiments, the serotonin receptor modulator is pimavanserin and the psychedelic is ibogaine, wherein pimavanserin is administered to post-treat between about 60 minutes and about 180 minutes post to the administration of ibogaine.

In some embodiments, the serotonin receptor modulator is nelotanserin and the psychedelic is ibogaine, wherein the nelotanserin is administered to post-treat at least 15 minutes post to the ibogaine. In some embodiments, the serotonin receptor modulator is nelotanserin and the psychedelic is ibogaine, wherein the nelotanserin is administered to post-treat at least 30 minutes post to the ibogaine. In some embodiments, the serotonin receptor modulator is nelotanserin and the psychedelic is ibogaine, wherein the nelotanserin is administered to post-treat at least 60 minutes post to the ibogaine. In some embodiments, the serotonin receptor modulator is nelotanserin and the psychedelic is ibogaine, wherein the nelotanserin is administered to post-treat at least 90 minutes post to the ibogaine. In some embodiments, the serotonin receptor modulator is nelotanserin and the psychedelic is ibogaine, wherein the nelotanserin is administered to post-treat at least 120 minutes post to the ibogaine. In some embodiments, the serotonin receptor modulator is nelotanserin and the psychedelic is ibogaine, wherein the nelotanserin is administered to post-treat at least 150 minutes post to the ibogaine. In some embodiments, the serotonin receptor modulator is nelotanserin and the psychedelic is ibogaine, wherein the nelotanserin is administered to post-treat between about 15 minutes and about 150 minutes post to the ibogaine. In some embodiments, the serotonin receptor modulator is nelotanserin and the psychedelic is ibogaine, wherein the nelotanserin is administered to post-treat at least 180 minutes post to the ibogaine. In some embodiments, the serotonin receptor modulator is nelotanserin and the psychedelic is ibogaine, wherein the nelotanserin is administered to post-treat at least 210 minutes post to the ibogaine. In some embodiments, the serotonin receptor modulator is nelotanserin and the psychedelic is ibogaine, wherein the nelotanserin is administered to post-treat at least 240 minutes post to the ibogaine. In some embodiments, the serotonin receptor modulator is nelotanserin and the psychedelic is ibogaine, wherein the nelotanserin is administered to post-treat at least 270 minutes post to the ibogaine. In some embodiments, the serotonin receptor modulator is nelotanserin and the psychedelic is ibogaine, wherein the nelotanserin is administered to post-treat at least 300 minutes post to the ibogaine. In some embodiments, the serotonin receptor modulator is nelotanserin and the psychedelic is ibogaine, wherein the nelotanserin is administered to post-treat at least 330 minutes post to the ibogaine. In some embodiments, the serotonin receptor modulator is nelotanserin and the psychedelic is ibogaine, wherein the nelotanserin is administered to post-treat at least 360 minutes post to the ibogaine. In some preferred embodiments, the serotonin receptor modulator is nelotanserin and the psychedelic is ibogaine, wherein nelotanserin is administered to post-treat between about 60 minutes and about 180 minutes post to the administration of ibogaine.

In some embodiments, the serotonin receptor modulator is pruvanserin and the psychedelic is ibogaine, wherein the pruvanserin is administered to post-treat at least 15 minutes post to the ibogaine. In some embodiments, the serotonin receptor modulator is pruvanserin and the psychedelic is ibogaine, wherein the pruvanserin is administered to post-treat at least 30 minutes post to the ibogaine. In some embodiments, the serotonin receptor modulator is pruvanserin and the psychedelic is ibogaine, wherein the pruvanserin is administered to post-treat at least 60 minutes post to the ibogaine. In some embodiments, the serotonin receptor modulator is pruvanserin and the psychedelic is ibogaine, wherein the pruvanserin is administered to post-treat at least 90 minutes post to the ibogaine. In some embodiments, the serotonin receptor modulator is pruvanserin and the psychedelic is ibogaine, wherein the pruvanserin is administered to post-treat at least 120 minutes post to the ibogaine. In some embodiments, the serotonin receptor modulator is pruvanserin and the psychedelic is ibogaine, wherein the pruvanserin is administered to post-treat at least 150 minutes post to the ibogaine. In some embodiments, the serotonin receptor modulator is pruvanserin and the psychedelic is ibogaine, wherein the pruvanserin is administered to post-treat between about 15 minutes and about 150 minutes post to the ibogaine. In some embodiments, the serotonin receptor modulator is pruvanserin and the psychedelic is ibogaine, wherein the pruvanserin is administered to post-treat at least 180 minutes post to the ibogaine. In some embodiments, the serotonin receptor modulator is pruvanserin and the psychedelic is ibogaine, wherein the pruvanserin is administered to post-treat at least 210 minutes post to the ibogaine. In some embodiments, the serotonin receptor modulator is pruvanserin and the psychedelic is ibogaine, wherein the pruvanserin is administered to post-treat at least 240 minutes post to the ibogaine. In some embodiments, the serotonin receptor modulator is pruvanserin and the psychedelic is ibogaine, wherein the pruvanserin is administered to post-treat at least 270 minutes post to the ibogaine. In some embodiments, the serotonin receptor modulator is pruvanserin and the psychedelic is ibogaine, wherein the pruvanserin is administered to post-treat at least 300 minutes post to the ibogaine. In some embodiments, the serotonin receptor modulator is pruvanserin and the psychedelic is ibogaine, wherein the pruvanserin is administered to post-treat at least 330 minutes post to the ibogaine. In some embodiments, the serotonin receptor modulator is pruvanserin and the psychedelic is ibogaine, wherein the pruvanserin is administered to post-treat at least 360 minutes post to the ibogaine. In some preferred embodiments, the serotonin receptor modulator is pruvanserin and the psychedelic is ibogaine, wherein pruvanserin is administered to post-treat between about 60 minutes and about 180 minutes post to the administration of ibogaine.

In some embodiments, the serotonin receptor modulator is flibanserin and the psychedelic is ibogaine, wherein the flibanserin is administered to post-treat at least 15 minutes post to the administration of ibogaine. In some embodiments, the serotonin receptor modulator is flibanserin and the psychedelic is ibogaine, wherein the flibanserin is administered to post-treat at least 30 minutes post to the ibogaine. In some embodiments, the serotonin receptor modulator is flibanserin and the psychedelic is ibogaine, wherein the flibanserin is administered to post-treat at least 60 minutes post to the ibogaine. In some embodiments, the serotonin receptor modulator is flibanserin and the psychedelic is ibogaine, wherein the flibanserin is administered to post-treat at least 90 minutes post to the ibogaine. In some embodiments, the serotonin receptor modulator is flibanserin and the psychedelic is ibogaine, wherein the flibanserin is administered to post-treat at least 120 minutes post to the ibogaine. In some embodiments, the serotonin receptor modulator is flibanserin and the psychedelic is ibogaine, wherein the flibanserin is administered to post-treat at least 150 minutes post to the ibogaine. In some embodiments, the serotonin receptor modulator is flibanserin and the psychedelic is ibogaine, wherein the flibanserin is administered to post-treat between about 15 minutes and about 150 minutes post to the ibogaine. In some embodiments, the serotonin receptor modulator is flibanserin and the psychedelic is ibogaine, wherein the flibanserin is administered to post-treat at least 180 minutes post to the ibogaine. In some embodiments, the serotonin receptor modulator is flibanserin and the psychedelic is ibogaine, wherein the flibanserin is administered to post-treat at least 210 minutes post to the ibogaine. In some embodiments, the serotonin receptor modulator is flibanserin and the psychedelic is ibogaine, wherein the flibanserin is administered to post-treat at least 240 minutes post to the ibogaine. In some embodiments, the serotonin receptor modulator is flibanserin and the psychedelic is ibogaine, wherein the flibanserin is administered to post-treat at least 270 minutes post to the ibogaine. In some embodiments, the serotonin receptor modulator is flibanserin and the psychedelic is ibogaine, wherein the flibanserin is administered to post-treat at least 300 minutes post to the ibogaine. In some embodiments, the serotonin receptor modulator is flibanserin and the psychedelic is ibogaine, wherein the flibanserin is administered to post-treat at least 330 minutes post to the ibogaine. In some embodiments, the serotonin receptor modulator is flibanserin and the psychedelic is ibogaine, wherein the flibanserin is administered to post-treat at least 360 minutes post to the ibogaine. In some preferred embodiments, the serotonin receptor modulator is flibanserin and the psychedelic is ibogaine, wherein flibanserin is administered to post-treat between about 60 minutes and about 180 minutes post to the administration of ibogaine.

In some embodiments, the serotonin receptor modulator is olanzapine and the psychedelic is ibogaine, wherein the olanzapine is administered to post-treat at least 15 minutes post to the administration of ibogaine. In some embodiments, the serotonin receptor modulator is olanzapine and the psychedelic is ibogaine, wherein the olanzapine is administered to post-treat at least 30 minutes post to the ibogaine. In some embodiments, the serotonin receptor modulator is olanzapine and the psychedelic is ibogaine, wherein the olanzapine is administered to post-treat at least 60 minutes post to the ibogaine. In some embodiments, the serotonin receptor modulator is olanzapine and the psychedelic is ibogaine, wherein the olanzapine is administered to post-treat at least 90 minutes post to the ibogaine. In some embodiments, the serotonin receptor modulator is olanzapine and the psychedelic is ibogaine, wherein the olanzapine is administered to post-treat at least 120 minutes post to the ibogaine. In some embodiments, the serotonin receptor modulator is olanzapine and the psychedelic is ibogaine, wherein the olanzapine is administered to post-treat at least 150 minutes post to the ibogaine. In some embodiments, the serotonin receptor modulator is olanzapine and the psychedelic is ibogaine, wherein the olanzapine is administered to post-treat between about 15 minutes and about 150 minutes post to the ibogaine. In some embodiments, the serotonin receptor modulator is olanzapine and the psychedelic is ibogaine, wherein the olanzapine is administered to post-treat at least 180 minutes post to the ibogaine. In some embodiments, the serotonin receptor modulator is olanzapine and the psychedelic is ibogaine, wherein the olanzapine is administered to post-treat at least 210 minutes post to the ibogaine. In some embodiments, the serotonin receptor modulator is olanzapine and the psychedelic is ibogaine, wherein the olanzapine is administered to post-treat at least 240 minutes post to the ibogaine. In some embodiments, the serotonin receptor modulator is olanzapine and the psychedelic is ibogaine, wherein the olanzapine is administered to post-treat at least 270 minutes post to the ibogaine. In some embodiments, the serotonin receptor modulator is olanzapine and the psychedelic is ibogaine, wherein the olanzapine is administered to post-treat at least 300 minutes post to the ibogaine. In some embodiments, the serotonin receptor modulator is olanzapine and the psychedelic is ibogaine, wherein the olanzapine is administered to post-treat at least 330 minutes post to the ibogaine. In some embodiments, the serotonin receptor modulator is olanzapine and the psychedelic is ibogaine, wherein the olanzapine is administered to post-treat at least 360 minutes post to the ibogaine. In some preferred embodiments, the serotonin receptor modulator is olanzapine and the psychedelic is ibogaine, wherein olanzapine is administered to post-treat between about 60 minutes and about 180 minutes post to the administration of ibogaine.

In some embodiments, the serotonin receptor modulator is quetiapine and the psychedelic is ibogaine, wherein the quetiapine is administered to post-treat at least 15 minutes post to the administration of ibogaine. In some embodiments, the serotonin receptor modulator is quetiapine and the psychedelic is ibogaine, wherein the quetiapine is administered to post-treat at least 30 minutes post to the ibogaine. In some embodiments, the serotonin receptor modulator is quetiapine and the psychedelic is ibogaine, wherein the quetiapine is administered to post-treat at least 60 minutes post to the ibogaine. In some embodiments, the serotonin receptor modulator is quetiapine and the psychedelic is ibogaine, wherein the quetiapine is administered to post-treat at least 90 minutes post to the ibogaine. In some embodiments, the serotonin receptor modulator is quetiapine and the psychedelic is ibogaine, wherein the quetiapine is administered to post-treat at least 120 minutes post to the ibogaine. In some embodiments, the serotonin receptor modulator is quetiapine and the psychedelic is ibogaine, wherein the quetiapine is administered to post-treat at least 150 minutes post to the ibogaine. In some embodiments, the serotonin receptor modulator is quetiapine and the psychedelic is ibogaine, wherein the quetiapine is administered to post-treat between about 15 minutes and about 150 minutes post to the ibogaine. In some embodiments, the serotonin receptor modulator is quetiapine and the psychedelic is ibogaine, wherein the quetiapine is administered to post-treat at least 180 minutes post to the ibogaine. In some embodiments, the serotonin receptor modulator is quetiapine and the psychedelic is ibogaine, wherein the quetiapine is administered to post-treat at least 210 minutes post to the ibogaine. In some embodiments, the serotonin receptor modulator is quetiapine and the psychedelic is ibogaine, wherein the quetiapine is administered to post-treat at least 240 minutes post to the ibogaine. In some embodiments, the serotonin receptor modulator is quetiapine and the psychedelic is ibogaine, wherein the quetiapine is administered to post-treat at least 270 minutes post to the ibogaine. In some embodiments, the serotonin receptor modulator is quetiapine and the psychedelic is ibogaine, wherein the quetiapine is administered to post-treat at least 300 minutes post to the ibogaine. In some embodiments, the serotonin receptor modulator is quetiapine and the psychedelic is ibogaine, wherein the quetiapine is administered to post-treat at least 330 minutes post to the ibogaine. In some embodiments, the serotonin receptor modulator is quetiapine and the psychedelic is ibogaine, wherein the quetiapine is administered to post-treat at least 360 minutes post to the ibogaine. In some preferred embodiments, the serotonin receptor modulator is quetiapine and the psychedelic is ibogaine, wherein quetiapine is administered to post-treat between about 60 minutes and about 180 minutes post to the administration of ibogaine.

In some embodiments, the serotonin receptor modulator is risperidone and the psychedelic is ibogaine, wherein the risperidone is administered to post-treat at least 15 minutes post to the administration of ibogaine. In some embodiments, the serotonin receptor modulator is risperidone and the psychedelic is ibogaine, wherein the risperidone is administered to post-treat at least 30 minutes post to the ibogaine. In some embodiments, the serotonin receptor modulator is risperidone and the psychedelic is ibogaine, wherein the risperidone is administered to post-treat at least 60 minutes post to the ibogaine. In some embodiments, the serotonin receptor modulator is risperidone and the psychedelic is ibogaine, wherein the risperidone is administered to post-treat at least 90 minutes post to the ibogaine. In some embodiments, the serotonin receptor modulator is risperidone and the psychedelic is ibogaine, wherein the risperidone is administered to post-treat at least 120 minutes post to the ibogaine. In some embodiments, the serotonin receptor modulator is risperidone and the psychedelic is ibogaine, wherein the risperidone is administered to post-treat at least 150 minutes post to the ibogaine. In some embodiments, the serotonin receptor modulator is risperidone and the psychedelic is ibogaine, wherein the risperidone is administered to post-treat between about 15 minutes and about 150 minutes post to the ibogaine. In some embodiments, the serotonin receptor modulator is risperidone and the psychedelic is ibogaine, wherein the risperidone is administered to post-treat at least 180 minutes post to the ibogaine. In some embodiments, the serotonin receptor modulator is risperidone and the psychedelic is ibogaine, wherein the risperidone is administered to post-treat at least 210 minutes post to the ibogaine. In some embodiments, the serotonin receptor modulator is risperidone and the psychedelic is ibogaine, wherein the risperidone is administered to post-treat at least 240 minutes post to the ibogaine. In some embodiments, the serotonin receptor modulator is risperidone and the psychedelic is ibogaine, wherein the risperidone is administered to post-treat at least 270 minutes post to the ibogaine. In some embodiments, the serotonin receptor modulator is risperidone and the psychedelic is ibogaine, wherein the risperidone is administered to post-treat at least 300 minutes post to the ibogaine. In some embodiments, the serotonin receptor modulator is risperidone and the psychedelic is ibogaine, wherein the risperidone is administered to post-treat at least 330 minutes post to the ibogaine. In some embodiments, the serotonin receptor modulator is risperidone and the psychedelic is ibogaine, wherein the risperidone is administered to post-treat at least 360 minutes post to the ibogaine. In some preferred embodiments, the serotonin receptor modulator is risperidone and the psychedelic is ibogaine, wherein risperidone is administered to post-treat between about 60 minutes and about 180 minutes post to the administration of ibogaine.

In some embodiments, the serotonin receptor modulator is eplivanserin and the psychedelic is MDMA, wherein the eplivanserin is administered to post-treat at least 15 minutes post to the administration of MDMA. In some embodiments, the serotonin receptor modulator is eplivanserin and the psychedelic is MDMA, wherein the eplivanserin is administered to post-treat at least 30 minutes post to the MDMA. In some embodiments, the serotonin receptor modulator is eplivanserin and the psychedelic is MDMA, wherein the eplivanserin is administered to post-treat at least 60 minutes post to the MDMA. In some embodiments, the serotonin receptor modulator is eplivanserin and the psychedelic is MDMA, wherein the eplivanserin is administered to post-treat at least 90 minutes post to the MDMA. In some embodiments, the serotonin receptor modulator is eplivanserin and the psychedelic is MDMA, wherein the eplivanserin is administered to post-treat at least 120 minutes post to the MDMA. In some embodiments, the serotonin receptor modulator is eplivanserin and the psychedelic is MDMA, wherein the eplivanserin is administered to post-treat at least 150 minutes post to the MDMA. In some embodiments, the serotonin receptor modulator is eplivanserin and the psychedelic is MDMA, wherein the eplivanserin is administered to post-treat between about 15 minutes and about 150 minutes post to the MDMA. In some embodiments, the serotonin receptor modulator is eplivanserin and the psychedelic is MDMA, wherein the eplivanserin is administered to post-treat at least 180 minutes post to the MDMA. In some embodiments, the serotonin receptor modulator is eplivanserin and the psychedelic is MDMA, wherein the eplivanserin is administered to post-treat at least 210 minutes post to the MDMA. In some embodiments, the serotonin receptor modulator is eplivanserin and the psychedelic is MDMA, wherein the eplivanserin is administered to post-treat at least 240 minutes post to the MDMA. In some embodiments, the serotonin receptor modulator is eplivanserin and the psychedelic is MDMA, wherein the eplivanserin is administered to post-treat at least 270 minutes post to the MDMA. In some embodiments, the serotonin receptor modulator is eplivanserin and the psychedelic is MDMA, wherein the eplivanserin is administered to post-treat at least 300 minutes post to the MDMA. In some embodiments, the serotonin receptor modulator is eplivanserin and the psychedelic is MDMA, wherein the eplivanserin is administered to post-treat at least 330 minutes post to the MDMA. In some embodiments, the serotonin receptor modulator is eplivanserin and the psychedelic is MDMA, wherein the eplivanserin is administered to post-treat at least 360 minutes post to the MDMA.

In some preferred embodiments, the serotonin receptor modulator is eplivanserin and the psychedelic is MDMA, wherein eplivanserin is administered to post-treat between about 60 minutes and about 180 minutes post to the administration of MDMA.

In some embodiments, the serotonin receptor modulator is volinanserin and the psychedelic is MDMA, wherein the volinanserin is administered to post-treat at least 15 minutes post to the administration of MDMA. In some embodiments, the serotonin receptor modulator is volinanserin and the psychedelic is MDMA, wherein the volinanserin is administered to post-treat at least 30 minutes post to the MDMA. In some embodiments, the serotonin receptor modulator is volinanserin and the psychedelic is MDMA, wherein the volinanserin is administered to post-treat at least 60 minutes post to the MDMA. In some embodiments, the serotonin receptor modulator is volinanserin and the psychedelic is MDMA, wherein the volinanserin is administered to post-treat at least 90 minutes post to the MDMA. In some embodiments, the serotonin receptor modulator is volinanserin and the psychedelic is MDMA, wherein the volinanserin is administered to post-treat at least 120 minutes post to the MDMA. In some embodiments, the serotonin receptor modulator is volinanserin and the psychedelic is MDMA, wherein the volinanserin is administered to post-treat at least 150 minutes post to the MDMA. In some embodiments, the serotonin receptor modulator is volinanserin and the psychedelic is MDMA, wherein the volinanserin is administered to post-treat between about 15 minutes and about 150 minutes post to the MDMA. In some embodiments, the serotonin receptor modulator is volinanserin and the psychedelic is MDMA, wherein the volinanserin is administered to post-treat at least 180 minutes post to the MDMA. In some embodiments, the serotonin receptor modulator is volinanserin and the psychedelic is MDMA, wherein the volinanserin is administered to post-treat at least 210 minutes post to the MDMA. In some embodiments, the serotonin receptor modulator is volinanserin and the psychedelic is MDMA, wherein the volinanserin is administered to post-treat at least 240 minutes post to the MDMA. In some embodiments, the serotonin receptor modulator is volinanserin and the psychedelic is MDMA, wherein the volinanserin is administered to post-treat at least 270 minutes post to the MDMA. In some embodiments, the serotonin receptor modulator is volinanserin and the psychedelic is MDMA, wherein the volinanserin is administered to post-treat at least 300 minutes post to the MDMA. In some embodiments, the serotonin receptor modulator is volinanserin and the psychedelic is MDMA, wherein the volinanserin is administered to post-treat at least 330 minutes post to the MDMA. In some embodiments, the serotonin receptor modulator is volinanserin and the psychedelic is MDMA, wherein the volinanserin is administered to post-treat at least 360 minutes post to the MDMA. In some preferred embodiments, the serotonin receptor modulator is volinanserin and the psychedelic is MDMA, wherein volinanserin is administered to post-treat between about 60 minutes and about 180 minutes post to the administration of MDMA.

In some embodiments, the serotonin receptor modulator is ketanserin and the psychedelic is MDMA, wherein the ketanserin is administered to post-treat at least 15 minutes post to the administration of MDMA. In some embodiments, the serotonin receptor modulator is ketanserin and the psychedelic is MDMA, wherein the ketanserin is administered to post-treat at least 30 minutes post to the MDMA. In some embodiments, the serotonin receptor modulator is ketanserin and the psychedelic is MDMA, wherein the ketanserin is administered to post-treat at least 60 minutes post to the MDMA. In some embodiments, the serotonin receptor modulator is ketanserin and the psychedelic is MDMA, wherein the ketanserin is administered to post-treat at least 90 minutes post to the MDMA. In some embodiments, the serotonin receptor modulator is ketanserin and the psychedelic is MDMA, wherein the ketanserin is administered to post-treat at least 120 minutes post to the MDMA. In some embodiments, the serotonin receptor modulator is ketanserin and the psychedelic is MDMA, wherein the ketanserin is administered to post-treat at least 150 minutes post to the MDMA. In some embodiments, the serotonin receptor modulator is ketanserin and the psychedelic is MDMA, wherein the ketanserin is administered to post-treat between about 15 minutes and about 150 minutes post to the MDMA. In some embodiments, the serotonin receptor modulator is ketanserin and the psychedelic is MDMA, wherein the ketanserin is administered to post-treat at least 180 minutes post to the MDMA. In some embodiments, the serotonin receptor modulator is ketanserin and the psychedelic is MDMA, wherein the ketanserin is administered to post-treat at least 210 minutes post to the MDMA. In some embodiments, the serotonin receptor modulator is ketanserin and the psychedelic is MDMA, wherein the ketanserin is administered to post-treat at least 240 minutes post to the MDMA. In some embodiments, the serotonin receptor modulator is ketanserin and the psychedelic is MDMA, wherein the ketanserin is administered to post-treat at least 270 minutes post to the MDMA. In some embodiments, the serotonin receptor modulator is ketanserin and the psychedelic is MDMA, wherein the ketanserin is administered to post-treat at least 300 minutes post to the MDMA. In some embodiments, the serotonin receptor modulator is ketanserin and the psychedelic is MDMA, wherein the ketanserin is administered to post-treat at least 330 minutes post to the MDMA. In some embodiments, the serotonin receptor modulator is ketanserin and the psychedelic is MDMA, wherein the ketanserin is administered to post-treat at least 360 minutes post to the MDMA. In some preferred embodiments, the serotonin receptor modulator is ketanserin and the psychedelic is MDMA, wherein ketanserin is administered to post-treat between about 60 minutes and about 180 minutes post to the administration of MDMA.

In some embodiments, the serotonin receptor modulator is ritanserin and the psychedelic is MDMA, wherein the ritanserin is administered to post-treat at least 15 minutes post to the MDMA. In some embodiments, the serotonin receptor modulator is ritanserin and the psychedelic is MDMA, wherein the ritanserin is administered to post-treat at least 30 minutes post to the MDMA. In some embodiments, the serotonin receptor modulator is ritanserin and the psychedelic is MDMA, wherein the ritanserin is administered to post-treat at least 60 minutes post to the MDMA. In some embodiments, the serotonin receptor modulator is ritanserin and the psychedelic is MDMA, wherein the ritanserin is administered to post-treat at least 90 minutes post to the MDMA. In some embodiments, the serotonin receptor modulator is ritanserin and the psychedelic is MDMA, wherein the ritanserin is administered to post-treat at least 120 minutes post to the MDMA. In some embodiments, the serotonin receptor modulator is ritanserin and the psychedelic is MDMA, wherein the ritanserin is administered to post-treat at least 150 minutes post to the MDMA. In some embodiments, the serotonin receptor modulator is ritanserin and the psychedelic is MDMA, wherein the ritanserin is administered to post-treat between about 15 minutes and about 150 minutes post to the MDMA. In some embodiments, the serotonin receptor modulator is ritanserin and the psychedelic is MDMA, wherein the ritanserin is administered to post-treat at least 180 minutes post to the MDMA. In some embodiments, the serotonin receptor modulator is ritanserin and the psychedelic is MDMA, wherein the ritanserin is administered to post-treat at least 210 minutes post to the MDMA. In some embodiments, the serotonin receptor modulator is ritanserin and the psychedelic is MDMA, wherein the ritanserin is administered to post-treat at least 240 minutes post to the MDMA. In some embodiments, the serotonin receptor modulator is ritanserin and the psychedelic is MDMA, wherein the ritanserin is administered to post-treat at least 270 minutes post to the MDMA. In some embodiments, the serotonin receptor modulator is ritanserin and the psychedelic is MDMA, wherein the ritanserin is administered to post-treat at least 300 minutes post to the MDMA. In some embodiments, the serotonin receptor modulator is ritanserin and the psychedelic is MDMA, wherein the ritanserin is administered to post-treat at least 330 minutes post to the MDMA. In some embodiments, the serotonin receptor modulator is ritanserin and the psychedelic is MDMA, wherein the ritanserin is administered to post-treat at least 360 minutes post to the MDMA. In some preferred embodiments, the serotonin receptor modulator is ritanserin and the psychedelic is MDMA, wherein ritanserin is administered to post-treat between about 60 minutes and about 180 minutes post to the administration of MDMA.

In some embodiments, the serotonin receptor modulator is pimavanserin and the psychedelic is MDMA, wherein the pimavanserin is administered to post-treat at least 15 minutes post to the MDMA. In some embodiments, the serotonin receptor modulator is pimavanserin and the psychedelic is MDMA, wherein the pimavanserin is administered to post-treat at least 30 minutes post to the MDMA. In some embodiments, the serotonin receptor modulator is pimavanserin and the psychedelic is MDMA, wherein the pimavanserin is administered to post-treat at least 60 minutes post to the MDMA. In some embodiments, the serotonin receptor modulator is pimavanserin and the psychedelic is MDMA, wherein the pimavanserin is administered to post-treat at least 90 minutes post to the MDMA. In some embodiments, the serotonin receptor modulator is pimavanserin and the psychedelic is MDMA, wherein the pimavanserin is administered to post-treat at least 120 minutes post to the MDMA. In some embodiments, the serotonin receptor modulator is pimavanserin and the psychedelic is MDMA, wherein the pimavanserin is administered to post-treat at least 150 minutes post to the MDMA. In some embodiments, the serotonin receptor modulator is pimavanserin and the psychedelic is MDMA, wherein the pimavanserin is administered to post-treat between about 15 minutes and about 150 minutes post to the MDMA. In some embodiments, the serotonin receptor modulator is pimavanserin and the psychedelic is MDMA, wherein the pimavanserin is administered to post-treat at least 180 minutes post to the MDMA. In some embodiments, the serotonin receptor modulator is pimavanserin and the psychedelic is MDMA, wherein the pimavanserin is administered to post-treat at least 210 minutes post to the MDMA. In some embodiments, the serotonin receptor modulator is pimavanserin and the psychedelic is MDMA, wherein the pimavanserin is administered to post-treat at least 240 minutes post to the MDMA. In some embodiments, the serotonin receptor modulator is pimavanserin and the psychedelic is MDMA, wherein the pimavanserin is administered to post-treat at least 270 minutes post to the MDMA. In some embodiments, the serotonin receptor modulator is pimavanserin and the psychedelic is MDMA, wherein the pimavanserin is administered to post-treat at least 300 minutes post to the MDMA. In some embodiments, the serotonin receptor modulator is pimavanserin and the psychedelic is MDMA, wherein the pimavanserin is administered to post-treat at least 330 minutes post to the MDMA. In some embodiments, the serotonin receptor modulator is pimavanserin and the psychedelic is MDMA, wherein the pimavanserin is administered to post-treat at least 360 minutes post to the MDMA. In some preferred embodiments, the serotonin receptor modulator is pimavanserin and the psychedelic is MDMA, wherein pimavanserin is administered to post-treat between about 60 minutes and about 180 minutes post to the administration of MDMA.

In some embodiments, the serotonin receptor modulator is nelotanserin and the psychedelic is MDMA, wherein the nelotanserin is administered to post-treat at least 15 minutes post to the MDMA. In some embodiments, the serotonin receptor modulator is nelotanserin and the psychedelic is MDMA, wherein the nelotanserin is administered to post-treat at least 30 minutes post to the MDMA. In some embodiments, the serotonin receptor modulator is nelotanserin and the psychedelic is MDMA, wherein the nelotanserin is administered to post-treat at least 60 minutes post to the MDMA. In some embodiments, the serotonin receptor modulator is nelotanserin and the psychedelic is MDMA, wherein the nelotanserin is administered to post-treat at least 90 minutes post to the MDMA. In some embodiments, the serotonin receptor modulator is nelotanserin and the psychedelic is MDMA, wherein the nelotanserin is administered to post-treat at least 120 minutes post to the MDMA. In some embodiments, the serotonin receptor modulator is nelotanserin and the psychedelic is MDMA, wherein the nelotanserin is administered to post-treat at least 150 minutes post to the MDMA. In some embodiments, the serotonin receptor modulator is nelotanserin and the psychedelic is MDMA, wherein the nelotanserin is administered to post-treat between about 15 minutes and about 150 minutes post to the MDMA. In some embodiments, the serotonin receptor modulator is nelotanserin and the psychedelic is MDMA, wherein the nelotanserin is administered to post-treat at least 180 minutes post to the MDMA. In some embodiments, the serotonin receptor modulator is nelotanserin and the psychedelic is MDMA, wherein the nelotanserin is administered to post-treat at least 210 minutes post to the MDMA. In some embodiments, the serotonin receptor modulator is nelotanserin and the psychedelic is MDMA, wherein the nelotanserin is administered to post-treat at least 240 minutes post to the MDMA. In some embodiments, the serotonin receptor modulator is nelotanserin and the psychedelic is MDMA, wherein the nelotanserin is administered to post-treat at least 270 minutes post to the MDMA. In some embodiments, the serotonin receptor modulator is nelotanserin and the psychedelic is MDMA, wherein the nelotanserin is administered to post-treat at least 300 minutes post to the MDMA. In some embodiments, the serotonin receptor modulator is nelotanserin and the psychedelic is MDMA, wherein the nelotanserin is administered to post-treat at least 330 minutes post to the MDMA. In some embodiments, the serotonin receptor modulator is nelotanserin and the psychedelic is MDMA, wherein the nelotanserin is administered to post-treat at least 360 minutes post to the MDMA. In some preferred embodiments, the serotonin receptor modulator is nelotanserin and the psychedelic is MDMA, wherein nelotanserin is administered to post-treat between about 60 minutes and about 180 minutes post to the administration of MDMA.

In some embodiments, the serotonin receptor modulator is pruvanserin and the psychedelic is MDMA, wherein the pruvanserin is administered to post-treat at least 15 minutes post to the MDMA. In some embodiments, the serotonin receptor modulator is pruvanserin and the psychedelic is MDMA, wherein the pruvanserin is administered to post-treat at least 30 minutes post to the MDMA. In some embodiments, the serotonin receptor modulator is pruvanserin and the psychedelic is MDMA, wherein the pruvanserin is administered to post-treat at least 60 minutes post to the MDMA. In some embodiments, the serotonin receptor modulator is pruvanserin and the psychedelic is MDMA, wherein the pruvanserin is administered to post-treat at least 90 minutes post to the MDMA. In some embodiments, the serotonin receptor modulator is pruvanserin and the psychedelic is MDMA, wherein the pruvanserin is administered to post-treat at least 120 minutes post to the MDMA. In some embodiments, the serotonin receptor modulator is pruvanserin and the psychedelic is MDMA, wherein the pruvanserin is administered to post-treat at least 150 minutes post to the MDMA. In some embodiments, the serotonin receptor modulator is pruvanserin and the psychedelic is MDMA, wherein the pruvanserin is administered to post-treat between about 15 minutes and about 150 minutes post to the MDMA. In some embodiments, the serotonin receptor modulator is pruvanserin and the psychedelic is MDMA, wherein the pruvanserin is administered to post-treat at least 180 minutes post to the MDMA. In some embodiments, the serotonin receptor modulator is pruvanserin and the psychedelic is MDMA, wherein the pruvanserin is administered to post-treat at least 210 minutes post to the MDMA. In some embodiments, the serotonin receptor modulator is pruvanserin and the psychedelic is MDMA, wherein the pruvanserin is administered to post-treat at least 240 minutes post to the MDMA. In some embodiments, the serotonin receptor modulator is pruvanserin and the psychedelic is MDMA, wherein the pruvanserin is administered to post-treat at least 270 minutes post to the MDMA. In some embodiments, the serotonin receptor modulator is pruvanserin and the psychedelic is MDMA, wherein the pruvanserin is administered to post-treat at least 300 minutes post to the MDMA. In some embodiments, the serotonin receptor modulator is pruvanserin and the psychedelic is MDMA, wherein the pruvanserin is administered to post-treat at least 330 minutes post to the MDMA. In some embodiments, the serotonin receptor modulator is pruvanserin and the psychedelic is MDMA, wherein the pruvanserin is administered to post-treat at least 360 minutes post to the MDMA. In some preferred embodiments, the serotonin receptor modulator is pruvanserin and the psychedelic is MDMA, wherein pruvanserin is administered to post-treat between about 60 minutes and about 180 minutes post to the administration of MDMA.

In some embodiments, the serotonin receptor modulator is flibanserin and the psychedelic is MDMA, wherein the flibanserin is administered to post-treat at least 15 minutes post to the administration of MDMA. In some embodiments, the serotonin receptor modulator is flibanserin and the psychedelic is MDMA, wherein the flibanserin is administered to post-treat at least 30 minutes post to the MDMA. In some embodiments, the serotonin receptor modulator is flibanserin and the psychedelic is MDMA, wherein the flibanserin is administered to post-treat at least 60 minutes post to the MDMA. In some embodiments, the serotonin receptor modulator is flibanserin and the psychedelic is MDMA, wherein the flibanserin is administered to post-treat at least 90 minutes post to the MDMA. In some embodiments, the serotonin receptor modulator is flibanserin and the psychedelic is MDMA, wherein the flibanserin is administered to post-treat at least 120 minutes post to the MDMA. In some embodiments, the serotonin receptor modulator is flibanserin and the psychedelic is MDMA, wherein the flibanserin is administered to post-treat at least 150 minutes post to the MDMA. In some embodiments, the serotonin receptor modulator is flibanserin and the psychedelic is MDMA, wherein the flibanserin is administered to post-treat between about 15 minutes and about 150 minutes post to the MDMA. In some embodiments, the serotonin receptor modulator is flibanserin and the psychedelic is MDMA, wherein the flibanserin is administered to post-treat at least 180 minutes post to the MDMA. In some embodiments, the serotonin receptor modulator is flibanserin and the psychedelic is MDMA, wherein the flibanserin is administered to post-treat at least 210 minutes post to the MDMA. In some embodiments, the serotonin receptor modulator is flibanserin and the psychedelic is MDMA, wherein the flibanserin is administered to post-treat at least 240 minutes post to the MDMA. In some embodiments, the serotonin receptor modulator is flibanserin and the psychedelic is MDMA, wherein the flibanserin is administered to post-treat at least 270 minutes post to the MDMA. In some embodiments, the serotonin receptor modulator is flibanserin and the psychedelic is MDMA, wherein the flibanserin is administered to post-treat at least 300 minutes post to the MDMA. In some embodiments, the serotonin receptor modulator is flibanserin and the psychedelic is MDMA, wherein the flibanserin is administered to post-treat at least 330 minutes post to the MDMA. In some embodiments, the serotonin receptor modulator is flibanserin and the psychedelic is MDMA, wherein the flibanserin is administered to post-treat at least 360 minutes post to the MDMA. In some preferred embodiments, the serotonin receptor modulator is flibanserin and the psychedelic is MDMA, wherein flibanserin is administered to post-treat between about 60 minutes and about 180 minutes post to the administration of MDMA.

In some embodiments, the serotonin receptor modulator is olanzapine and the psychedelic is MDMA, wherein the olanzapine is administered to post-treat at least 15 minutes post to the administration of MDMA. In some embodiments, the serotonin receptor modulator is olanzapine and the psychedelic is MDMA, wherein the olanzapine is administered to post-treat at least 30 minutes post to the MDMA. In some embodiments, the serotonin receptor modulator is olanzapine and the psychedelic is MDMA, wherein the olanzapine is administered to post-treat at least 60 minutes post to the MDMA. In some embodiments, the serotonin receptor modulator is olanzapine and the psychedelic is MDMA, wherein the olanzapine is administered to post-treat at least 90 minutes post to the MDMA. In some embodiments, the serotonin receptor modulator is olanzapine and the psychedelic is MDMA, wherein the olanzapine is administered to post-treat at least 120 minutes post to the MDMA. In some embodiments, the serotonin receptor modulator is olanzapine and the psychedelic is MDMA, wherein the olanzapine is administered to post-treat at least 150 minutes post to the MDMA. In some embodiments, the serotonin receptor modulator is olanzapine and the psychedelic is MDMA, wherein the olanzapine is administered to post-treat between about 15 minutes and about 150 minutes post to the MDMA. In some embodiments, the serotonin receptor modulator is olanzapine and the psychedelic is MDMA, wherein the olanzapine is administered to post-treat at least 180 minutes post to the MDMA. In some embodiments, the serotonin receptor modulator is olanzapine and the psychedelic is MDMA, wherein the olanzapine is administered to post-treat at least 210 minutes post to the MDMA. In some embodiments, the serotonin receptor modulator is olanzapine and the psychedelic is MDMA, wherein the olanzapine is administered to post-treat at least 240 minutes post to the MDMA. In some embodiments, the serotonin receptor modulator is olanzapine and the psychedelic is MDMA, wherein the olanzapine is administered to post-treat at least 270 minutes post to the MDMA. In some embodiments, the serotonin receptor modulator is olanzapine and the psychedelic is MDMA, wherein the olanzapine is administered to post-treat at least 300 minutes post to the MDMA. In some embodiments, the serotonin receptor modulator is olanzapine and the psychedelic is MDMA, wherein the olanzapine is administered to post-treat at least 330 minutes post to the MDMA. In some embodiments, the serotonin receptor modulator is olanzapine and the psychedelic is MDMA, wherein the olanzapine is administered to post-treat at least 360 minutes post to the MDMA. In some preferred embodiments, the serotonin receptor modulator is olanzapine and the psychedelic is MDMA, wherein olanzapine is administered to post-treat between about 60 minutes and about 180 minutes post to the administration of MDMA.

In some embodiments, the serotonin receptor modulator is quetiapine and the psychedelic is MDMA, wherein the quetiapine is administered to post-treat at least 15 minutes post to the administration of MDMA. In some embodiments, the serotonin receptor modulator is quetiapine and the psychedelic is MDMA, wherein the quetiapine is administered to post-treat at least 30 minutes post to the MDMA. In some embodiments, the serotonin receptor modulator is quetiapine and the psychedelic is MDMA, wherein the quetiapine is administered to post-treat at least 60 minutes post to the MDMA. In some embodiments, the serotonin receptor modulator is quetiapine and the psychedelic is MDMA, wherein the quetiapine is administered to post-treat at least 90 minutes post to the MDMA. In some embodiments, the serotonin receptor modulator is quetiapine and the psychedelic is MDMA, wherein the quetiapine is administered to post-treat at least 120 minutes post to the MDMA. In some embodiments, the serotonin receptor modulator is quetiapine and the psychedelic is MDMA, wherein the quetiapine is administered to post-treat at least 150 minutes post to the MDMA. In some embodiments, the serotonin receptor modulator is quetiapine and the psychedelic is MDMA, wherein the quetiapine is administered to post-treat between about 15 minutes and about 150 minutes post to the MDMA. In some embodiments, the serotonin receptor modulator is quetiapine and the psychedelic is MDMA, wherein the quetiapine is administered to post-treat at least 180 minutes post to the MDMA. In some embodiments, the serotonin receptor modulator is quetiapine and the psychedelic is MDMA, wherein the quetiapine is administered to post-treat at least 210 minutes post to the MDMA. In some embodiments, the serotonin receptor modulator is quetiapine and the psychedelic is MDMA, wherein the quetiapine is administered to post-treat at least 240 minutes post to the MDMA. In some embodiments, the serotonin receptor modulator is quetiapine and the psychedelic is MDMA, wherein the quetiapine is administered to post-treat at least 270 minutes post to the MDMA. In some embodiments, the serotonin receptor modulator is quetiapine and the psychedelic is MDMA, wherein the quetiapine is administered to post-treat at least 300 minutes post to the MDMA. In some embodiments, the serotonin receptor modulator is quetiapine and the psychedelic is MDMA, wherein the quetiapine is administered to post-treat at least 330 minutes post to the MDMA. In some embodiments, the serotonin receptor modulator is quetiapine and the psychedelic is MDMA, wherein the quetiapine is administered to post-treat at least 360 minutes post to the MDMA. In some preferred embodiments, the serotonin receptor modulator is quetiapine and the psychedelic is MDMA, wherein quetiapine is administered to post-treat between about 60 minutes and about 180 minutes post to the administration of MDMA.

In some embodiments, the serotonin receptor modulator is risperidone and the psychedelic is MDMA, wherein the risperidone is administered to post-treat at least 15 minutes post to the administration of MDMA. In some embodiments, the serotonin receptor modulator is risperidone and the psychedelic is MDMA, wherein the risperidone is administered to post-treat at least 30 minutes post to the MDMA. In some embodiments, the serotonin receptor modulator is risperidone and the psychedelic is MDMA, wherein the risperidone is administered to post-treat at least 60 minutes post to the MDMA. In some embodiments, the serotonin receptor modulator is risperidone and the psychedelic is MDMA, wherein the risperidone is administered to post-treat at least 90 minutes post to the MDMA. In some embodiments, the serotonin receptor modulator is risperidone and the psychedelic is MDMA, wherein the risperidone is administered to post-treat at least 120 minutes post to the MDMA. In some embodiments, the serotonin receptor modulator is risperidone and the psychedelic is MDMA, wherein the risperidone is administered to post-treat at least 150 minutes post to the MDMA. In some embodiments, the serotonin receptor modulator is risperidone and the psychedelic is MDMA, wherein the risperidone is administered to post-treat between about 15 minutes and about 150 minutes post to the MDMA. In some embodiments, the serotonin receptor modulator is risperidone and the psychedelic is MDMA, wherein the risperidone is administered to post-treat at least 180 minutes post to the MDMA. In some embodiments, the serotonin receptor modulator is risperidone and the psychedelic is MDMA, wherein the risperidone is administered to post-treat at least 210 minutes post to the MDMA. In some embodiments, the serotonin receptor modulator is risperidone and the psychedelic is MDMA, wherein the risperidone is administered to post-treat at least 240 minutes post to the MDMA. In some embodiments, the serotonin receptor modulator is risperidone and the psychedelic is MDMA, wherein the risperidone is administered to post-treat at least 270 minutes post to the MDMA. In some embodiments, the serotonin receptor modulator is risperidone and the psychedelic is MDMA, wherein the risperidone is administered to post-treat at least 300 minutes post to the MDMA. In some embodiments, the serotonin receptor modulator is risperidone and the psychedelic is MDMA, wherein the risperidone is administered to post-treat at least 330 minutes post to the MDMA. In some embodiments, the serotonin receptor modulator is risperidone and the psychedelic is MDMA, wherein the risperidone is administered to post-treat at least 360 minutes post to the MDMA. In some preferred embodiments, the serotonin receptor modulator is risperidone and the psychedelic is MDMA, wherein risperidone is administered to post-treat between about 60 minutes and about 180 minutes post to the administration of MDMA.

In some embodiments, the serotonin receptor modulator is eplivanserin and the psychedelic is DOM, wherein the eplivanserin is administered to post-treat at least 15 minutes post to the administration of DOM. In some embodiments, the serotonin receptor modulator is eplivanserin and the psychedelic is DOM, wherein the eplivanserin is administered to post-treat at least 30 minutes post to the DOM. In some embodiments, the serotonin receptor modulator is eplivanserin and the psychedelic is DOM, wherein the eplivanserin is administered to post-treat at least 60 minutes post to the DOM. In some embodiments, the serotonin receptor modulator is eplivanserin and the psychedelic is DOM, wherein the eplivanserin is administered to post-treat at least 90 minutes post to the DOM. In some embodiments, the serotonin receptor modulator is eplivanserin and the psychedelic is DOM, wherein the eplivanserin is administered to post-treat at least 120 minutes post to the DOM. In some embodiments, the serotonin receptor modulator is eplivanserin and the psychedelic is DOM, wherein the eplivanserin is administered to post-treat at least 150 minutes post to the DOM. In some embodiments, the serotonin receptor modulator is eplivanserin and the psychedelic is DOM, wherein the eplivanserin is administered to post-treat between about 15 minutes and about 150 minutes post to the DOM. In some embodiments, the serotonin receptor modulator is eplivanserin and the psychedelic is DOM, wherein the eplivanserin is administered to post-treat at least 180 minutes post to the DOM. In some embodiments, the serotonin receptor modulator is eplivanserin and the psychedelic is DOM, wherein the eplivanserin is administered to post-treat at least 210 minutes post to the DOM. In some embodiments, the serotonin receptor modulator is eplivanserin and the psychedelic is DOM, wherein the eplivanserin is administered to post-treat at least 240 minutes post to the DOM. In some embodiments, the serotonin receptor modulator is eplivanserin and the psychedelic is DOM, wherein the eplivanserin is administered to post-treat at least 270 minutes post to the DOM. In some embodiments, the serotonin receptor modulator is eplivanserin and the psychedelic is DOM, wherein the eplivanserin is administered to post-treat at least 300 minutes post to the DOM. In some embodiments, the serotonin receptor modulator is eplivanserin and the psychedelic is DOM, wherein the eplivanserin is administered to post-treat at least 330 minutes post to the DOM. In some embodiments, the serotonin receptor modulator is eplivanserin and the psychedelic is DOM, wherein the eplivanserin is administered to post-treat at least 360 minutes post to the DOM.

In some preferred embodiments, the serotonin receptor modulator is eplivanserin and the psychedelic is DOM, wherein eplivanserin is administered to post-treat between about 60 minutes and about 180 minutes post to the administration of DOM.

In some embodiments, the serotonin receptor modulator is volinanserin and the psychedelic is DOM, wherein the volinanserin is administered to post-treat at least 15 minutes post to the administration of DOM. In some embodiments, the serotonin receptor modulator is volinanserin and the psychedelic is DOM, wherein the volinanserin is administered to post-treat at least 30 minutes post to the DOM. In some embodiments, the serotonin receptor modulator is volinanserin and the psychedelic is DOM, wherein the volinanserin is administered to post-treat at least 60 minutes post to the DOM. In some embodiments, the serotonin receptor modulator is volinanserin and the psychedelic is DOM, wherein the volinanserin is administered to post-treat at least 90 minutes post to the DOM. In some embodiments, the serotonin receptor modulator is volinanserin and the psychedelic is DOM, wherein the volinanserin is administered to post-treat at least 120 minutes post to the DOM. In some embodiments, the serotonin receptor modulator is volinanserin and the psychedelic is DOM, wherein the volinanserin is administered to post-treat at least 150 minutes post to the DOM. In some embodiments, the serotonin receptor modulator is volinanserin and the psychedelic is DOM, wherein the volinanserin is administered to post-treat between about 15 minutes and about 150 minutes post to the DOM. In some embodiments, the serotonin receptor modulator is volinanserin and the psychedelic is DOM, wherein the volinanserin is administered to post-treat at least 180 minutes post to the DOM. In some embodiments, the serotonin receptor modulator is volinanserin and the psychedelic is DOM, wherein the volinanserin is administered to post-treat at least 210 minutes post to the DOM. In some embodiments, the serotonin receptor modulator is volinanserin and the psychedelic is DOM, wherein the volinanserin is administered to post-treat at least 240 minutes post to the DOM. In some embodiments, the serotonin receptor modulator is volinanserin and the psychedelic is DOM, wherein the volinanserin is administered to post-treat at least 270 minutes post to the DOM. In some embodiments, the serotonin receptor modulator is volinanserin and the psychedelic is DOM, wherein the volinanserin is administered to post-treat at least 300 minutes post to the DOM. In some embodiments, the serotonin receptor modulator is volinanserin and the psychedelic is DOM, wherein the volinanserin is administered to post-treat at least 330 minutes post to the DOM. In some embodiments, the serotonin receptor modulator is volinanserin and the psychedelic is DOM, wherein the volinanserin is administered to post-treat at least 360 minutes post to the DOM. In some preferred embodiments, the serotonin receptor modulator is volinanserin and the psychedelic is DOM, wherein volinanserin is administered to post-treat between about 60 minutes and about 180 minutes post to the administration of DOM.

In some embodiments, the serotonin receptor modulator is ketanserin and the psychedelic is DOM, wherein the ketanserin is administered to post-treat at least 15 minutes post to the administration of DOM. In some embodiments, the serotonin receptor modulator is ketanserin and the psychedelic is DOM, wherein the ketanserin is administered to post-treat at least 30 minutes post to the DOM. In some embodiments, the serotonin receptor modulator is ketanserin and the psychedelic is DOM, wherein the ketanserin is administered to post-treat at least 60 minutes post to the DOM. In some embodiments, the serotonin receptor modulator is ketanserin and the psychedelic is DOM, wherein the ketanserin is administered to post-treat at least 90 minutes post to the DOM. In some embodiments, the serotonin receptor modulator is ketanserin and the psychedelic is DOM, wherein the ketanserin is administered to post-treat at least 120 minutes post to the DOM. In some embodiments, the serotonin receptor modulator is ketanserin and the psychedelic is DOM, wherein the ketanserin is administered to post-treat at least 150 minutes post to the DOM. In some embodiments, the serotonin receptor modulator is ketanserin and the psychedelic is DOM, wherein the ketanserin is administered to post-treat between about 15 minutes and about 150 minutes post to the DOM. In some embodiments, the serotonin receptor modulator is ketanserin and the psychedelic is DOM, wherein the ketanserin is administered to post-treat at least 180 minutes post to the DOM. In some embodiments, the serotonin receptor modulator is ketanserin and the psychedelic is DOM, wherein the ketanserin is administered to post-treat at least 210 minutes post to the DOM. In some embodiments, the serotonin receptor modulator is ketanserin and the psychedelic is DOM, wherein the ketanserin is administered to post-treat at least 240 minutes post to the DOM. In some embodiments, the serotonin receptor modulator is ketanserin and the psychedelic is DOM, wherein the ketanserin is administered to post-treat at least 270 minutes post to the DOM. In some embodiments, the serotonin receptor modulator is ketanserin and the psychedelic is DOM, wherein the ketanserin is administered to post-treat at least 300 minutes post to the DOM. In some embodiments, the serotonin receptor modulator is ketanserin and the psychedelic is DOM, wherein the ketanserin is administered to post-treat at least 330 minutes post to the DOM. In some embodiments, the serotonin receptor modulator is ketanserin and the psychedelic is DOM, wherein the ketanserin is administered to post-treat at least 360 minutes post to the DOM. In some preferred embodiments, the serotonin receptor modulator is ketanserin and the psychedelic is DOM, wherein ketanserin is administered to post-treat between about 60 minutes and about 180 minutes post to the administration of DOM.

In some embodiments, the serotonin receptor modulator is ritanserin and the psychedelic is DOM, wherein the ritanserin is administered to post-treat at least 15 minutes post to the DOM. In some embodiments, the serotonin receptor modulator is ritanserin and the psychedelic is DOM, wherein the ritanserin is administered to post-treat at least 30 minutes post to the DOM. In some embodiments, the serotonin receptor modulator is ritanserin and the psychedelic is DOM, wherein the ritanserin is administered to post-treat at least 60 minutes post to the DOM. In some embodiments, the serotonin receptor modulator is ritanserin and the psychedelic is DOM, wherein the ritanserin is administered to post-treat at least 90 minutes post to the DOM. In some embodiments, the serotonin receptor modulator is ritanserin and the psychedelic is DOM, wherein the ritanserin is administered to post-treat at least 120 minutes post to the DOM. In some embodiments, the serotonin receptor modulator is ritanserin and the psychedelic is DOM, wherein the ritanserin is administered to post-treat at least 150 minutes post to the DOM. In some embodiments, the serotonin receptor modulator is ritanserin and the psychedelic is DOM, wherein the ritanserin is administered to post-treat between about 15 minutes and about 150 minutes post to the DOM. In some embodiments, the serotonin receptor modulator is ritanserin and the psychedelic is DOM, wherein the ritanserin is administered to post-treat at least 180 minutes post to the DOM. In some embodiments, the serotonin receptor modulator is ritanserin and the psychedelic is DOM, wherein the ritanserin is administered to post-treat at least 210 minutes post to the DOM. In some embodiments, the serotonin receptor modulator is ritanserin and the psychedelic is DOM, wherein the ritanserin is administered to post-treat at least 240 minutes post to the DOM. In some embodiments, the serotonin receptor modulator is ritanserin and the psychedelic is DOM, wherein the ritanserin is administered to post-treat at least 270 minutes post to the DOM. In some embodiments, the serotonin receptor modulator is ritanserin and the psychedelic is DOM, wherein the ritanserin is administered to post-treat at least 300 minutes post to the DOM. In some embodiments, the serotonin receptor modulator is ritanserin and the psychedelic is DOM, wherein the ritanserin is administered to post-treat at least 330 minutes post to the DOM. In some embodiments, the serotonin receptor modulator is ritanserin and the psychedelic is DOM, wherein the ritanserin is administered to post-treat at least 360 minutes post to the DOM. In some preferred embodiments, the serotonin receptor modulator is ritanserin and the psychedelic is DOM, wherein ritanserin is administered to post-treat between about 60 minutes and about 180 minutes post to the administration of DOM.

In some embodiments, the serotonin receptor modulator is pimavanserin and the psychedelic is DOM, wherein the pimavanserin is administered to post-treat at least 15 minutes post to the DOM. In some embodiments, the serotonin receptor modulator is pimavanserin and the psychedelic is DOM, wherein the pimavanserin is administered to post-treat at least 30 minutes post to the DOM. In some embodiments, the serotonin receptor modulator is pimavanserin and the psychedelic is DOM, wherein the pimavanserin is administered to post-treat at least 60 minutes post to the DOM. In some embodiments, the serotonin receptor modulator is pimavanserin and the psychedelic is DOM, wherein the pimavanserin is administered to post-treat at least 90 minutes post to the DOM. In some embodiments, the serotonin receptor modulator is pimavanserin and the psychedelic is DOM, wherein the pimavanserin is administered to post-treat at least 120 minutes post to the DOM. In some embodiments, the serotonin receptor modulator is pimavanserin and the psychedelic is DOM, wherein the pimavanserin is administered to post-treat at least 150 minutes post to the DOM. In some embodiments, the serotonin receptor modulator is pimavanserin and the psychedelic is DOM, wherein the pimavanserin is administered to post-treat between about 15 minutes and about 150 minutes post to the DOM. In some embodiments, the serotonin receptor modulator is pimavanserin and the psychedelic is DOM, wherein the pimavanserin is administered to post-treat at least 180 minutes post to the DOM. In some embodiments, the serotonin receptor modulator is pimavanserin and the psychedelic is DOM, wherein the pimavanserin is administered to post-treat at least 210 minutes post to the DOM. In some embodiments, the serotonin receptor modulator is pimavanserin and the psychedelic is DOM, wherein the pimavanserin is administered to post-treat at least 240 minutes post to the DOM. In some embodiments, the serotonin receptor modulator is pimavanserin and the psychedelic is DOM, wherein the pimavanserin is administered to post-treat at least 270 minutes post to the DOM. In some embodiments, the serotonin receptor modulator is pimavanserin and the psychedelic is DOM, wherein the pimavanserin is administered to post-treat at least 300 minutes post to the DOM. In some embodiments, the serotonin receptor modulator is pimavanserin and the psychedelic is DOM, wherein the pimavanserin is administered to post-treat at least 330 minutes post to the DOM. In some embodiments, the serotonin receptor modulator is pimavanserin and the psychedelic is DOM, wherein the pimavanserin is administered to post-treat at least 360 minutes post to the DOM. In some preferred embodiments, the serotonin receptor modulator is pimavanserin and the psychedelic is DOM, wherein pimavanserin is administered to post-treat between about 60 minutes and about 180 minutes post to the administration of DOM.

In some embodiments, the serotonin receptor modulator is nelotanserin and the psychedelic is DOM, wherein the nelotanserin is administered to post-treat at least 15 minutes post to the DOM. In some embodiments, the serotonin receptor modulator is nelotanserin and the psychedelic is DOM, wherein the nelotanserin is administered to post-treat at least 30 minutes post to the DOM. In some embodiments, the serotonin receptor modulator is nelotanserin and the psychedelic is DOM, wherein the nelotanserin is administered to post-treat at least 60 minutes post to the DOM. In some embodiments, the serotonin receptor modulator is nelotanserin and the psychedelic is DOM, wherein the nelotanserin is administered to post-treat at least 90 minutes post to the DOM. In some embodiments, the serotonin receptor modulator is nelotanserin and the psychedelic is DOM, wherein the nelotanserin is administered to post-treat at least 120 minutes post to the DOM. In some embodiments, the serotonin receptor modulator is nelotanserin and the psychedelic is DOM, wherein the nelotanserin is administered to post-treat at least 150 minutes post to the DOM. In some embodiments, the serotonin receptor modulator is nelotanserin and the psychedelic is DOM, wherein the nelotanserin is administered to post-treat between about 15 minutes and about 150 minutes post to the DOM. In some embodiments, the serotonin receptor modulator is nelotanserin and the psychedelic is DOM, wherein the nelotanserin is administered to post-treat at least 180 minutes post to the DOM. In some embodiments, the serotonin receptor modulator is nelotanserin and the psychedelic is DOM, wherein the nelotanserin is administered to post-treat at least 210 minutes post to the DOM. In some embodiments, the serotonin receptor modulator is nelotanserin and the psychedelic is DOM, wherein the nelotanserin is administered to post-treat at least 240 minutes post to the DOM. In some embodiments, the serotonin receptor modulator is nelotanserin and the psychedelic is DOM, wherein the nelotanserin is administered to post-treat at least 270 minutes post to the DOM. In some embodiments, the serotonin receptor modulator is nelotanserin and the psychedelic is DOM, wherein the nelotanserin is administered to post-treat at least 300 minutes post to the DOM. In some embodiments, the serotonin receptor modulator is nelotanserin and the psychedelic is DOM, wherein the nelotanserin is administered to post-treat at least 330 minutes post to the DOM. In some embodiments, the serotonin receptor modulator is nelotanserin and the psychedelic is DOM, wherein the nelotanserin is administered to post-treat at least 360 minutes post to the DOM. In some preferred embodiments, the serotonin receptor modulator is nelotanserin and the psychedelic is DOM, wherein nelotanserin is administered to post-treat between about 60 minutes and about 180 minutes post to the administration of DOM.

In some embodiments, the serotonin receptor modulator is pruvanserin and the psychedelic is DOM, wherein the pruvanserin is administered to post-treat at least 15 minutes post to the DOM. In some embodiments, the serotonin receptor modulator is pruvanserin and the psychedelic is DOM, wherein the pruvanserin is administered to post-treat at least 30 minutes post to the DOM. In some embodiments, the serotonin receptor modulator is pruvanserin and the psychedelic is DOM, wherein the pruvanserin is administered to post-treat at least 60 minutes post to the DOM. In some embodiments, the serotonin receptor modulator is pruvanserin and the psychedelic is DOM, wherein the pruvanserin is administered to post-treat at least 90 minutes post to the DOM. In some embodiments, the serotonin receptor modulator is pruvanserin and the psychedelic is DOM, wherein the pruvanserin is administered to post-treat at least 120 minutes post to the DOM. In some embodiments, the serotonin receptor modulator is pruvanserin and the psychedelic is DOM, wherein the pruvanserin is administered to post-treat at least 150 minutes post to the DOM. In some embodiments, the serotonin receptor modulator is pruvanserin and the psychedelic is DOM, wherein the pruvanserin is administered to post-treat between about 15 minutes and about 150 minutes post to the DOM. In some embodiments, the serotonin receptor modulator is pruvanserin and the psychedelic is DOM, wherein the pruvanserin is administered to post-treat at least 180 minutes post to the DOM. In some embodiments, the serotonin receptor modulator is pruvanserin and the psychedelic is DOM, wherein the pruvanserin is administered to post-treat at least 210 minutes post to the DOM. In some embodiments, the serotonin receptor modulator is pruvanserin and the psychedelic is DOM, wherein the pruvanserin is administered to post-treat at least 240 minutes post to the DOM. In some embodiments, the serotonin receptor modulator is pruvanserin and the psychedelic is DOM, wherein the pruvanserin is administered to post-treat at least 270 minutes post to the DOM. In some embodiments, the serotonin receptor modulator is pruvanserin and the psychedelic is DOM, wherein the pruvanserin is administered to post-treat at least 300 minutes post to the DOM. In some embodiments, the serotonin receptor modulator is pruvanserin and the psychedelic is DOM, wherein the pruvanserin is administered to post-treat at least 330 minutes post to the DOM. In some embodiments, the serotonin receptor modulator is pruvanserin and the psychedelic is DOM, wherein the pruvanserin is administered to post-treat at least 360 minutes post to the DOM. In some preferred embodiments, the serotonin receptor modulator is pruvanserin and the psychedelic is DOM, wherein pruvanserin is administered to post-treat between about 60 minutes and about 180 minutes post to the administration of DOM.

In some embodiments, the serotonin receptor modulator is flibanserin and the psychedelic is DOM, wherein the flibanserin is administered to post-treat at least 15 minutes post to the administration of DOM. In some embodiments, the serotonin receptor modulator is flibanserin and the psychedelic is DOM, wherein the flibanserin is administered to post-treat at least 30 minutes post to the DOM. In some embodiments, the serotonin receptor modulator is flibanserin and the psychedelic is DOM, wherein the flibanserin is administered to post-treat at least 60 minutes post to the DOM. In some embodiments, the serotonin receptor modulator is flibanserin and the psychedelic is DOM, wherein the flibanserin is administered to post-treat at least 90 minutes post to the DOM. In some embodiments, the serotonin receptor modulator is flibanserin and the psychedelic is DOM, wherein the flibanserin is administered to post-treat at least 120 minutes post to the DOM. In some embodiments, the serotonin receptor modulator is flibanserin and the psychedelic is DOM, wherein the flibanserin is administered to post-treat at least 150 minutes post to the DOM. In some embodiments, the serotonin receptor modulator is flibanserin and the psychedelic is DOM, wherein the flibanserin is administered to post-treat between about 15 minutes and about 150 minutes post to the DOM. In some embodiments, the serotonin receptor modulator is flibanserin and the psychedelic is DOM, wherein the flibanserin is administered to post-treat at least 180 minutes post to the DOM. In some embodiments, the serotonin receptor modulator is flibanserin and the psychedelic is DOM, wherein the flibanserin is administered to post-treat at least 210 minutes post to the DOM. In some embodiments, the serotonin receptor modulator is flibanserin and the psychedelic is DOM, wherein the flibanserin is administered to post-treat at least 240 minutes post to the DOM. In some embodiments, the serotonin receptor modulator is flibanserin and the psychedelic is DOM, wherein the flibanserin is administered to post-treat at least 270 minutes post to the DOM. In some embodiments, the serotonin receptor modulator is flibanserin and the psychedelic is DOM, wherein the flibanserin is administered to post-treat at least 300 minutes post to the DOM. In some embodiments, the serotonin receptor modulator is flibanserin and the psychedelic is DOM, wherein the flibanserin is administered to post-treat at least 330 minutes post to the DOM. In some embodiments, the serotonin receptor modulator is flibanserin and the psychedelic is DOM, wherein the flibanserin is administered to post-treat at least 360 minutes post to the DOM. In some preferred embodiments, the serotonin receptor modulator is flibanserin and the psychedelic is DOM, wherein flibanserin is administered to post-treat between about 60 minutes and about 180 minutes post to the administration of DOM.

In some embodiments, the serotonin receptor modulator is olanzapine and the psychedelic is DOM, wherein the olanzapine is administered to post-treat at least 15 minutes post to the administration of DOM. In some embodiments, the serotonin receptor modulator is olanzapine and the psychedelic is DOM, wherein the olanzapine is administered to post-treat at least 30 minutes post to the DOM. In some embodiments, the serotonin receptor modulator is olanzapine and the psychedelic is DOM, wherein the olanzapine is administered to post-treat at least 60 minutes post to the DOM. In some embodiments, the serotonin receptor modulator is olanzapine and the psychedelic is DOM, wherein the olanzapine is administered to post-treat at least 90 minutes post to the DOM. In some embodiments, the serotonin receptor modulator is olanzapine and the psychedelic is DOM, wherein the olanzapine is administered to post-treat at least 120 minutes post to the DOM. In some embodiments, the serotonin receptor modulator is olanzapine and the psychedelic is DOM, wherein the olanzapine is administered to post-treat at least 150 minutes post to the DOM. In some embodiments, the serotonin receptor modulator is olanzapine and the psychedelic is DOM, wherein the olanzapine is administered to post-treat between about 15 minutes and about 150 minutes post to the DOM. In some embodiments, the serotonin receptor modulator is olanzapine and the psychedelic is DOM, wherein the olanzapine is administered to post-treat at least 180 minutes post to the DOM. In some embodiments, the serotonin receptor modulator is olanzapine and the psychedelic is DOM, wherein the olanzapine is administered to post-treat at least 210 minutes post to the DOM. In some embodiments, the serotonin receptor modulator is olanzapine and the psychedelic is DOM, wherein the olanzapine is administered to post-treat at least 240 minutes post to the DOM. In some embodiments, the serotonin receptor modulator is olanzapine and the psychedelic is DOM, wherein the olanzapine is administered to post-treat at least 270 minutes post to the DOM. In some embodiments, the serotonin receptor modulator is olanzapine and the psychedelic is DOM, wherein the olanzapine is administered to post-treat at least 300 minutes post to the DOM. In some embodiments, the serotonin receptor modulator is olanzapine and the psychedelic is DOM, wherein the olanzapine is administered to post-treat at least 330 minutes post to the DOM. In some embodiments, the serotonin receptor modulator is olanzapine and the psychedelic is DOM, wherein the olanzapine is administered to post-treat at least 360 minutes post to the DOM. In some preferred embodiments, the serotonin receptor modulator is olanzapine and the psychedelic is DOM, wherein olanzapine is administered to post-treat between about 60 minutes and about 180 minutes post to the administration of DOM.

In some embodiments, the serotonin receptor modulator is quetiapine and the psychedelic is DOM, wherein the quetiapine is administered to post-treat at least 15 minutes post to the administration of DOM. In some embodiments, the serotonin receptor modulator is quetiapine and the psychedelic is DOM, wherein the quetiapine is administered to post-treat at least 30 minutes post to the DOM. In some embodiments, the serotonin receptor modulator is quetiapine and the psychedelic is DOM, wherein the quetiapine is administered to post-treat at least 60 minutes post to the DOM. In some embodiments, the serotonin receptor modulator is quetiapine and the psychedelic is DOM, wherein the quetiapine is administered to post-treat at least 90 minutes post to the DOM. In some embodiments, the serotonin receptor modulator is quetiapine and the psychedelic is DOM, wherein the quetiapine is administered to post-treat at least 120 minutes post to the DOM. In some embodiments, the serotonin receptor modulator is quetiapine and the psychedelic is DOM, wherein the quetiapine is administered to post-treat at least 150 minutes post to the DOM. In some embodiments, the serotonin receptor modulator is quetiapine and the psychedelic is DOM, wherein the quetiapine is administered to post-treat between about 15 minutes and about 150 minutes post to the DOM. In some embodiments, the serotonin receptor modulator is quetiapine and the psychedelic is DOM, wherein the quetiapine is administered to post-treat at least 180 minutes post to the DOM. In some embodiments, the serotonin receptor modulator is quetiapine and the psychedelic is DOM, wherein the quetiapine is administered to post-treat at least 210 minutes post to the DOM. In some embodiments, the serotonin receptor modulator is quetiapine and the psychedelic is DOM, wherein the quetiapine is administered to post-treat at least 240 minutes post to the DOM. In some embodiments, the serotonin receptor modulator is quetiapine and the psychedelic is DOM, wherein the quetiapine is administered to post-treat at least 270 minutes post to the DOM. In some embodiments, the serotonin receptor modulator is quetiapine and the psychedelic is DOM, wherein the quetiapine is administered to post-treat at least 300 minutes post to the DOM. In some embodiments, the serotonin receptor modulator is quetiapine and the psychedelic is DOM, wherein the quetiapine is administered to post-treat at least 330 minutes post to the DOM. In some embodiments, the serotonin receptor modulator is quetiapine and the psychedelic is DOM, wherein the quetiapine is administered to post-treat at least 360 minutes post to the DOM. In some preferred embodiments, the serotonin receptor modulator is quetiapine and the psychedelic is DOM, wherein quetiapine is administered to post-treat between about 60 minutes and about 180 minutes post to the administration of DOM.

In some embodiments, the serotonin receptor modulator is risperidone and the psychedelic is DOM, wherein the risperidone is administered to post-treat at least 15 minutes post to the administration of DOM. In some embodiments, the serotonin receptor modulator is risperidone and the psychedelic is DOM, wherein the risperidone is administered to post-treat at least 30 minutes post to the DOM. In some embodiments, the serotonin receptor modulator is risperidone and the psychedelic is DOM, wherein the risperidone is administered to post-treat at least 60 minutes post to the DOM. In some embodiments, the serotonin receptor modulator is risperidone and the psychedelic is DOM, wherein the risperidone is administered to post-treat at least 90 minutes post to the DOM. In some embodiments, the serotonin receptor modulator is risperidone and the psychedelic is DOM, wherein the risperidone is administered to post-treat at least 120 minutes post to the DOM. In some embodiments, the serotonin receptor modulator is risperidone and the psychedelic is DOM, wherein the risperidone is administered to post-treat at least 150 minutes post to the DOM. In some embodiments, the serotonin receptor modulator is risperidone and the psychedelic is DOM, wherein the risperidone is administered to post-treat between about 15 minutes and about 150 minutes post to the DOM. In some embodiments, the serotonin receptor modulator is risperidone and the psychedelic is DOM, wherein the risperidone is administered to post-treat at least 180 minutes post to the DOM. In some embodiments, the serotonin receptor modulator is risperidone and the psychedelic is DOM, wherein the risperidone is administered to post-treat at least 210 minutes post to the DOM. In some embodiments, the serotonin receptor modulator is risperidone and the psychedelic is DOM, wherein the risperidone is administered to post-treat at least 240 minutes post to the DOM. In some embodiments, the serotonin receptor modulator is risperidone and the psychedelic is DOM, wherein the risperidone is administered to post-treat at least 270 minutes post to the DOM. In some embodiments, the serotonin receptor modulator is risperidone and the psychedelic is DOM, wherein the risperidone is administered to post-treat at least 300 minutes post to the DOM. In some embodiments, the serotonin receptor modulator is risperidone and the psychedelic is DOM, wherein the risperidone is administered to post-treat at least 330 minutes post to the DOM. In some embodiments, the serotonin receptor modulator is risperidone and the psychedelic is DOM, wherein the risperidone is administered to post-treat at least 360 minutes post to the DOM. In some preferred embodiments, the serotonin receptor modulator is risperidone and the psychedelic is DOM, wherein risperidone is administered to post-treat between about 60 minutes and about 180 minutes post to the administration of DOM.

In some embodiments, the serotonin receptor modulator is eplivanserin and the psychedelic is mescaline, wherein eplivanserin is administered to post-treat at least 15 minutes post to the administration of mescaline. In some embodiments, the serotonin receptor modulator is eplivanserin and the psychedelic is mescaline, wherein the eplivanserin is administered to post-treat at least 30 minutes post to the mescaline. In some embodiments, the serotonin receptor modulator is eplivanserin and the psychedelic is mescaline, wherein the eplivanserin is administered to post-treat at least 60 minutes post to the mescaline. In some embodiments, the serotonin receptor modulator is eplivanserin and the psychedelic is mescaline, wherein the eplivanserin is administered to post-treat at least 90 minutes post to the mescaline. In some embodiments, the serotonin receptor modulator is eplivanserin and the psychedelic is mescaline, wherein the eplivanserin is administered to post-treat at least 120 minutes post to the mescaline. In some embodiments, the serotonin receptor modulator is eplivanserin and the psychedelic is mescaline, wherein the eplivanserin is administered to post-treat at least 150 minutes post to the mescaline. In some embodiments, the serotonin receptor modulator is eplivanserin and the psychedelic is mescaline, wherein the eplivanserin is administered to post-treat between about 15 minutes and about 150 minutes post to the mescaline. In some embodiments, the serotonin receptor modulator is eplivanserin and the psychedelic is mescaline, wherein the eplivanserin is administered to post-treat at least 180 minutes post to the mescaline. In some embodiments, the serotonin receptor modulator is eplivanserin and the psychedelic is mescaline, wherein the eplivanserin is administered to post-treat at least 210 minutes post to the mescaline. In some embodiments, the serotonin receptor modulator is eplivanserin and the psychedelic is mescaline, wherein the eplivanserin is administered to post-treat at least 240 minutes post to the mescaline. In some embodiments, the serotonin receptor modulator is eplivanserin and the psychedelic is mescaline, wherein the eplivanserin is administered to post-treat at least 270 minutes post to the mescaline. In some embodiments, the serotonin receptor modulator is eplivanserin and the psychedelic is mescaline, wherein the eplivanserin is administered to post-treat at least 300 minutes post to the mescaline. In some embodiments, the serotonin receptor modulator is eplivanserin and the psychedelic is mescaline, wherein the eplivanserin is administered to post-treat at least 330 minutes post to the mescaline. In some embodiments, the serotonin receptor modulator is eplivanserin and the psychedelic is mescaline, wherein the eplivanserin is administered to post-treat at least 360 minutes post to the mescaline. In some preferred embodiments, the serotonin receptor modulator is eplivanserin and the psychedelic is mescaline, wherein eplivanserin is administered to post-treat between about 60 minutes and about 180 minutes post to the administration of mescaline.

In some embodiments, the serotonin receptor modulator is volinanserin and the psychedelic is mescaline, wherein volinanserin is administered to post-treat at least 15 minutes post to the administration of mescaline. In some embodiments, the serotonin receptor modulator is volinanserin and the psychedelic is mescaline, wherein the volinanserin is administered to post-treat at least 30 minutes post to the mescaline. In some embodiments, the serotonin receptor modulator is volinanserin and the psychedelic is mescaline, wherein the volinanserin is administered to post-treat at least 60 minutes post to the mescaline. In some embodiments, the serotonin receptor modulator is volinanserin and the psychedelic is mescaline, wherein the volinanserin is administered to post-treat at least 90 minutes post to the mescaline. In some embodiments, the serotonin receptor modulator is volinanserin and the psychedelic is mescaline, wherein the volinanserin is administered to post-treat at least 120 minutes post to the mescaline. In some embodiments, the serotonin receptor modulator is volinanserin and the psychedelic is mescaline, wherein the volinanserin is administered to post-treat at least 150 minutes post to the mescaline. In some embodiments, the serotonin receptor modulator is volinanserin and the psychedelic is mescaline, wherein the volinanserin is administered to post-treat between about 15 minutes and about 150 minutes post to the mescaline. In some embodiments, the serotonin receptor modulator is volinanserin and the psychedelic is mescaline, wherein the volinanserin is administered to post-treat at least 180 minutes post to the mescaline. In some embodiments, the serotonin receptor modulator is volinanserin and the psychedelic is mescaline, wherein the volinanserin is administered to post-treat at least 210 minutes post to the mescaline. In some embodiments, the serotonin receptor modulator is volinanserin and the psychedelic is mescaline, wherein the volinanserin is administered to post-treat at least 240 minutes post to the mescaline. In some embodiments, the serotonin receptor modulator is volinanserin and the psychedelic is mescaline, wherein the volinanserin is administered to post-treat at least 270 minutes post to the mescaline. In some embodiments, the serotonin receptor modulator is volinanserin and the psychedelic is mescaline, wherein the volinanserin is administered to post-treat at least 300 minutes post to the mescaline. In some embodiments, the serotonin receptor modulator is volinanserin and the psychedelic is mescaline, wherein the volinanserin is administered to post-treat at least 330 minutes post to the mescaline. In some embodiments, the serotonin receptor modulator is volinanserin and the psychedelic is mescaline, wherein the volinanserin is administered to post-treat at least 360 minutes post to the mescaline. In some preferred embodiments, the serotonin receptor modulator is volinanserin and the psychedelic is mescaline, wherein volinanserin is administered to post-treat between about 60 minutes and about 180 minutes post to the administration of mescaline.

In some embodiments, the serotonin receptor modulator is ketanserin and the psychedelic is mescaline, wherein ketanserin is administered to post-treat at least 15 minutes post to the administration of mescaline. In some embodiments, the serotonin receptor modulator is ketanserin and the psychedelic is mescaline, wherein the ketanserin is administered to post-treat at least 30 minutes post to the mescaline. In some embodiments, the serotonin receptor modulator is ketanserin and the psychedelic is mescaline, wherein the ketanserin is administered to post-treat at least 60 minutes post to the mescaline. In some embodiments, the serotonin receptor modulator is ketanserin and the psychedelic is mescaline, wherein the ketanserin is administered to post-treat at least 90 minutes post to the mescaline. In some embodiments, the serotonin receptor modulator is ketanserin and the psychedelic is mescaline, wherein the ketanserin is administered to post-treat at least 120 minutes post to the mescaline. In some embodiments, the serotonin receptor modulator is ketanserin and the psychedelic is mescaline, wherein the ketanserin is administered to post-treat at least 150 minutes post to the mescaline. In some embodiments, the serotonin receptor modulator is ketanserin and the psychedelic is mescaline, wherein the ketanserin is administered to post-treat between about 15 minutes and about 150 minutes post to the mescaline. In some embodiments, the serotonin receptor modulator is ketanserin and the psychedelic is mescaline, wherein the ketanserin is administered to post-treat at least 180 minutes post to the mescaline. In some embodiments, the serotonin receptor modulator is ketanserin and the psychedelic is mescaline, wherein the ketanserin is administered to post-treat at least 210 minutes post to the mescaline. In some embodiments, the serotonin receptor modulator is ketanserin and the psychedelic is mescaline, wherein the ketanserin is administered to post-treat at least 240 minutes post to the mescaline. In some embodiments, the serotonin receptor modulator is ketanserin and the psychedelic is mescaline, wherein the ketanserin is administered to post-treat at least 270 minutes post to the mescaline. In some embodiments, the serotonin receptor modulator is ketanserin and the psychedelic is mescaline, wherein the ketanserin is administered to post-treat at least 300 minutes post to the mescaline. In some embodiments, the serotonin receptor modulator is ketanserin and the psychedelic is mescaline, wherein the ketanserin is administered to post-treat at least 330 minutes post to the mescaline. In some embodiments, the serotonin receptor modulator is ketanserin and the psychedelic is mescaline, wherein the ketanserin is administered to post-treat at least 360 minutes post to the mescaline. In some preferred embodiments, the serotonin receptor modulator is ketanserin and the psychedelic is mescaline, wherein ketanserin is administered to post-treat between about 60 minutes and about 180 minutes post to the administration of mescaline.

In some embodiments, the serotonin receptor modulator is ritanserin and the psychedelic is mescaline, wherein ritanserin is administered to post-treat at least 15 minutes post to the administration of mescaline. In some embodiments, the serotonin receptor modulator is ritanserin and the psychedelic is mescaline, wherein the ritanserin is administered to post-treat at least 30 minutes post to the mescaline. In some embodiments, the serotonin receptor modulator is ritanserin and the psychedelic is mescaline, wherein the ritanserin is administered to post-treat at least 60 minutes post to the mescaline. In some embodiments, the serotonin receptor modulator is ritanserin and the psychedelic is mescaline, wherein the ritanserin is administered to post-treat at least 90 minutes post to the mescaline. In some embodiments, the serotonin receptor modulator is ritanserin and the psychedelic is mescaline, wherein the ritanserin is administered to post-treat at least 120 minutes post to the mescaline. In some embodiments, the serotonin receptor modulator is ritanserin and the psychedelic is mescaline, wherein the ritanserin is administered to post-treat at least 150 minutes post to the mescaline. In some embodiments, the serotonin receptor modulator is ritanserin and the psychedelic is mescaline, wherein the ritanserin is administered to post-treat between about 15 minutes and about 150 minutes post to the mescaline. In some embodiments, the serotonin receptor modulator is ritanserin and the psychedelic is mescaline, wherein the ritanserin is administered to post-treat at least 180 minutes post to the mescaline. In some embodiments, the serotonin receptor modulator is ritanserin and the psychedelic is mescaline, wherein the ritanserin is administered to post-treat at least 210 minutes post to the mescaline. In some embodiments, the serotonin receptor modulator is ritanserin and the psychedelic is mescaline, wherein the ritanserin is administered to post-treat at least 240 minutes post to the mescaline. In some embodiments, the serotonin receptor modulator is ritanserin and the psychedelic is mescaline, wherein the ritanserin is administered to post-treat at least 270 minutes post to the mescaline. In some embodiments, the serotonin receptor modulator is ritanserin and the psychedelic is mescaline, wherein the ritanserin is administered to post-treat at least 300 minutes post to the mescaline. In some embodiments, the serotonin receptor modulator is ritanserin and the psychedelic is mescaline, wherein the ritanserin is administered to post-treat at least 330 minutes post to the mescaline. In some embodiments, the serotonin receptor modulator is ritanserin and the psychedelic is mescaline, wherein the ritanserin is administered to post-treat at least 360 minutes post to the mescaline. In some preferred embodiments, the serotonin receptor modulator is ritanserin and the psychedelic is mescaline, wherein ritanserin is administered to post-treat between about 60 minutes and about 180 minutes post to the administration of mescaline.

In some embodiments, the serotonin receptor modulator is pimavanserin and the psychedelic is mescaline, wherein pimavanserin is administered to post-treat at least 15 minutes post to the administration of mescaline. In some embodiments, the serotonin receptor modulator is pimavanserin and the psychedelic is mescaline, wherein the pimavanserin is administered to post-treat at least 30 minutes post to the mescaline. In some embodiments, the serotonin receptor modulator is pimavanserin and the psychedelic is mescaline, wherein the pimavanserin is administered to post-treat at least 60 minutes post to the mescaline. In some embodiments, the serotonin receptor modulator is pimavanserin and the psychedelic is mescaline, wherein the pimavanserin is administered to post-treat at least 90 minutes post to the mescaline. In some embodiments, the serotonin receptor modulator is pimavanserin and the psychedelic is mescaline, wherein the pimavanserin is administered to post-treat at least 120 minutes post to the mescaline. In some embodiments, the serotonin receptor modulator is pimavanserin and the psychedelic is mescaline, wherein the pimavanserin is administered to post-treat at least 150 minutes post to the mescaline. In some embodiments, the serotonin receptor modulator is pimavanserin and the psychedelic is mescaline, wherein the pimavanserin is administered to post-treat between about 15 minutes and about 150 minutes post to the mescaline. In some embodiments, the serotonin receptor modulator is pimavanserin and the psychedelic is mescaline, wherein the pimavanserin is administered to post-treat at least 180 minutes post to the mescaline. In some embodiments, the serotonin receptor modulator is pimavanserin and the psychedelic is mescaline, wherein the pimavanserin is administered to post-treat at least 210 minutes post to the mescaline. In some embodiments, the serotonin receptor modulator is pimavanserin and the psychedelic is mescaline, wherein the pimavanserin is administered to post-treat at least 240 minutes post to the mescaline. In some embodiments, the serotonin receptor modulator is pimavanserin and the psychedelic is mescaline, wherein the pimavanserin is administered to post-treat at least 270 minutes post to the mescaline. In some embodiments, the serotonin receptor modulator is pimavanserin and the psychedelic is mescaline, wherein the pimavanserin is administered to post-treat at least 300 minutes post to the mescaline. In some embodiments, the serotonin receptor modulator is pimavanserin and the psychedelic is mescaline, wherein the pimavanserin is administered to post-treat at least 330 minutes post to the mescaline. In some embodiments, the serotonin receptor modulator is pimavanserin and the psychedelic is mescaline, wherein the pimavanserin is administered to post-treat at least 360 minutes post to the mescaline. In some preferred embodiments, the serotonin receptor modulator is pimavanserin and the psychedelic is mescaline, wherein pimavanserin is administered to post-treat between about 60 minutes and about 180 minutes post to the administration of mescaline.

In some embodiments, the serotonin receptor modulator is nelotanserin and the psychedelic is mescaline, wherein nelotanserin is administered to post-treat at least 15 minutes post to the administration of mescaline. In some embodiments, the serotonin receptor modulator is nelotanserin and the psychedelic is mescaline, wherein the nelotanserin is administered to post-treat at least 30 minutes post to the mescaline. In some embodiments, the serotonin receptor modulator is nelotanserin and the psychedelic is mescaline, wherein the nelotanserin is administered to post-treat at least 60 minutes post to the mescaline. In some embodiments, the serotonin receptor modulator is nelotanserin and the psychedelic is mescaline, wherein the nelotanserin is administered to post-treat at least 90 minutes post to the mescaline. In some embodiments, the serotonin receptor modulator is nelotanserin and the psychedelic is mescaline, wherein the nelotanserin is administered to post-treat at least 120 minutes post to the mescaline. In some embodiments, the serotonin receptor modulator is nelotanserin and the psychedelic is mescaline, wherein the nelotanserin is administered to post-treat at least 150 minutes post to the mescaline. In some embodiments, the serotonin receptor modulator is nelotanserin and the psychedelic is mescaline, wherein the nelotanserin is administered to post-treat between about 15 minutes and about 150 minutes post to the mescaline. In some embodiments, the serotonin receptor modulator is nelotanserin and the psychedelic is mescaline, wherein the nelotanserin is administered to post-treat at least 180 minutes post to the mescaline. In some embodiments, the serotonin receptor modulator is nelotanserin and the psychedelic is mescaline, wherein the nelotanserin is administered to post-treat at least 210 minutes post to the mescaline. In some embodiments, the serotonin receptor modulator is nelotanserin and the psychedelic is mescaline, wherein the nelotanserin is administered to post-treat at least 240 minutes post to the mescaline. In some embodiments, the serotonin receptor modulator is nelotanserin and the psychedelic is mescaline, wherein the nelotanserin is administered to post-treat at least 270 minutes post to the mescaline. In some embodiments, the serotonin receptor modulator is nelotanserin and the psychedelic is mescaline, wherein the nelotanserin is administered to post-treat at least 300 minutes post to the mescaline. In some embodiments, the serotonin receptor modulator is nelotanserin and the psychedelic is mescaline, wherein the nelotanserin is administered to post-treat at least 330 minutes post to the mescaline. In some embodiments, the serotonin receptor modulator is nelotanserin and the psychedelic is mescaline, wherein the nelotanserin is administered to post-treat at least 360 minutes post to the mescaline. In some preferred embodiments, the serotonin receptor modulator is nelotanserin and the psychedelic is mescaline, wherein nelotanserin is administered to post-treat between about 60 minutes and about 180 minutes post to the administration of mescaline.

In some embodiments, the serotonin receptor modulator is pruvanserin and the psychedelic is mescaline, wherein pruvanserin is administered to post-treat at least 15 minutes post to the administration of mescaline. In some embodiments, the serotonin receptor modulator is pruvanserin and the psychedelic is mescaline, wherein the pruvanserin is administered to post-treat at least 30 minutes post to the mescaline. In some embodiments, the serotonin receptor modulator is pruvanserin and the psychedelic is mescaline, wherein the pruvanserin is administered to post-treat at least 60 minutes post to the mescaline. In some embodiments, the serotonin receptor modulator is pruvanserin and the psychedelic is mescaline, wherein the pruvanserin is administered to post-treat at least 90 minutes post to the mescaline. In some embodiments, the serotonin receptor modulator is pruvanserin and the psychedelic is mescaline, wherein the pruvanserin is administered to post-treat at least 120 minutes post to the mescaline. In some embodiments, the serotonin receptor modulator is pruvanserin and the psychedelic is mescaline, wherein the pruvanserin is administered to post-treat at least 150 minutes post to the mescaline. In some embodiments, the serotonin receptor modulator is pruvanserin and the psychedelic is mescaline, wherein the pruvanserin is administered to post-treat between about 15 minutes and about 150 minutes post to the mescaline. In some embodiments, the serotonin receptor modulator is pruvanserin and the psychedelic is mescaline, wherein the pruvanserin is administered to post-treat at least 180 minutes post to the mescaline. In some embodiments, the serotonin receptor modulator is pruvanserin and the psychedelic is mescaline, wherein the pruvanserin is administered to post-treat at least 210 minutes post to the mescaline. In some embodiments, the serotonin receptor modulator is pruvanserin and the psychedelic is mescaline, wherein the pruvanserin is administered to post-treat at least 240 minutes post to the mescaline. In some embodiments, the serotonin receptor modulator is pruvanserin and the psychedelic is mescaline, wherein the pruvanserin is administered to post-treat at least 270 minutes post to the mescaline. In some embodiments, the serotonin receptor modulator is pruvanserin and the psychedelic is mescaline, wherein the pruvanserin is administered to post-treat at least 300 minutes post to the mescaline. In some embodiments, the serotonin receptor modulator is pruvanserin and the psychedelic is mescaline, wherein the pruvanserin is administered to post-treat at least 330 minutes post to the mescaline. In some embodiments, the serotonin receptor modulator is pruvanserin and the psychedelic is mescaline, wherein the pruvanserin is administered to post-treat at least 360 minutes post to the mescaline. In some preferred embodiments, the serotonin receptor modulator is pruvanserin and the psychedelic is mescaline, wherein pruvanserin is administered to post-treat between about 60 minutes and about 180 minutes post to the administration of mescaline.

In some embodiments, the serotonin receptor modulator is flibanserin and the psychedelic is mescaline, wherein the flibanserin is administered to post-treat at least 15 minutes post to the administration of mescaline. In some embodiments, the serotonin receptor modulator is flibanserin and the psychedelic is mescaline, wherein the flibanserin is administered to post-treat at least 30 minutes post to the mescaline. In some embodiments, the serotonin receptor modulator is flibanserin and the psychedelic is mescaline, wherein the flibanserin is administered to post-treat at least 60 minutes post to the mescaline. In some embodiments, the serotonin receptor modulator is flibanserin and the psychedelic is mescaline, wherein the flibanserin is administered to post-treat at least 90 minutes post to the mescaline. In some embodiments, the serotonin receptor modulator is flibanserin and the psychedelic is mescaline, wherein the flibanserin is administered to post-treat at least 120 minutes post to the mescaline. In some embodiments, the serotonin receptor modulator is flibanserin and the psychedelic is mescaline, wherein the flibanserin is administered to post-treat at least 150 minutes post to the mescaline. In some embodiments, the serotonin receptor modulator is flibanserin and the psychedelic is mescaline, wherein the flibanserin is administered to post-treat between about 15 minutes and about 150 minutes post to the mescaline. In some embodiments, the serotonin receptor modulator is flibanserin and the psychedelic is mescaline, wherein the flibanserin is administered to post-treat at least 180 minutes post to the mescaline. In some embodiments, the serotonin receptor modulator is flibanserin and the psychedelic is mescaline, wherein the flibanserin is administered to post-treat at least 210 minutes post to the mescaline. In some embodiments, the serotonin receptor modulator is flibanserin and the psychedelic is mescaline, wherein the flibanserin is administered to post-treat at least 240 minutes post to the mescaline. In some embodiments, the serotonin receptor modulator is flibanserin and the psychedelic is mescaline, wherein the flibanserin is administered to post-treat at least 270 minutes post to the mescaline. In some embodiments, the serotonin receptor modulator is flibanserin and the psychedelic is mescaline, wherein the flibanserin is administered to post-treat at least 300 minutes post to the mescaline. In some embodiments, the serotonin receptor modulator is flibanserin and the psychedelic is mescaline, wherein the flibanserin is administered to post-treat at least 330 minutes post to the mescaline. In some embodiments, the serotonin receptor modulator is flibanserin and the psychedelic is mescaline, wherein the flibanserin is administered to post-treat at least 360 minutes post to the mescaline. In some preferred embodiments, the serotonin receptor modulator is flibanserin and the psychedelic is mescaline, wherein flibanserin is administered to post-treat between about 60 minutes and about 180 minutes post to the administration of mescaline.

In some embodiments, the serotonin receptor modulator is olanzapine and the psychedelic is mescaline, wherein olanzapine is administered to post-treat at least 15 minutes post to the administration of mescaline. In some embodiments, the serotonin receptor modulator is olanzapine and the psychedelic is mescaline, wherein the olanzapine is administered to post-treat at least 30 minutes post to the mescaline. In some embodiments, the serotonin receptor modulator is olanzapine and the psychedelic is mescaline, wherein the olanzapine is administered to post-treat at least 60 minutes post to the mescaline. In some embodiments, the serotonin receptor modulator is olanzapine and the psychedelic is mescaline, wherein the olanzapine is administered to post-treat at least 90 minutes post to the mescaline. In some embodiments, the serotonin receptor modulator is olanzapine and the psychedelic is mescaline, wherein the olanzapine is administered to post-treat at least 120 minutes post to the mescaline. In some embodiments, the serotonin receptor modulator is olanzapine and the psychedelic is mescaline, wherein the olanzapine is administered to post-treat at least 150 minutes post to the mescaline. In some embodiments, the serotonin receptor modulator is olanzapine and the psychedelic is mescaline, wherein the olanzapine is administered to post-treat between about 15 minutes and about 150 minutes post to the mescaline. In some embodiments, the serotonin receptor modulator is olanzapine and the psychedelic is mescaline, wherein the olanzapine is administered to post-treat at least 180 minutes post to the mescaline. In some embodiments, the serotonin receptor modulator is olanzapine and the psychedelic is mescaline, wherein the olanzapine is administered to post-treat at least 210 minutes post to the mescaline. In some embodiments, the serotonin receptor modulator is olanzapine and the psychedelic is mescaline, wherein the olanzapine is administered to post-treat at least 240 minutes post to the mescaline. In some embodiments, the serotonin receptor modulator is olanzapine and the psychedelic is mescaline, wherein the olanzapine is administered to post-treat at least 270 minutes post to the mescaline. In some embodiments, the serotonin receptor modulator is olanzapine and the psychedelic is mescaline, wherein the olanzapine is administered to post-treat at least 300 minutes post to the mescaline. In some embodiments, the serotonin receptor modulator is olanzapine and the psychedelic is mescaline, wherein the olanzapine is administered to post-treat at least 330 minutes post to the mescaline. In some embodiments, the serotonin receptor modulator is olanzapine and the psychedelic is mescaline, wherein the olanzapine is administered to post-treat at least 360 minutes post to the mescaline. In some preferred embodiments, the serotonin receptor modulator is olanzapine and the psychedelic is mescaline, wherein olanzapine is administered to post-treat between about 60 minutes and about 180 minutes post to the administration of mescaline.

In some embodiments, the serotonin receptor modulator is quetiapine and the psychedelic is mescaline, wherein quetiapine is administered to post-treat at least 15 minutes post to the administration of mescaline. In some embodiments, the serotonin receptor modulator is quetiapine and the psychedelic is mescaline, wherein the quetiapine is administered to post-treat at least 30 minutes post to the mescaline. In some embodiments, the serotonin receptor modulator is quetiapine and the psychedelic is mescaline, wherein the quetiapine is administered to post-treat at least 60 minutes post to the mescaline. In some embodiments, the serotonin receptor modulator is quetiapine and the psychedelic is mescaline, wherein the quetiapine is administered to post-treat at least 90 minutes post to the mescaline. In some embodiments, the serotonin receptor modulator is quetiapine and the psychedelic is mescaline, wherein the quetiapine is administered to post-treat at least 120 minutes post to the mescaline. In some embodiments, the serotonin receptor modulator is quetiapine and the psychedelic is mescaline, wherein the quetiapine is administered to post-treat at least 150 minutes post to the mescaline. In some embodiments, the serotonin receptor modulator is quetiapine and the psychedelic is mescaline, wherein the quetiapine is administered to post-treat between about 15 minutes and about 150 minutes post to the mescaline. In some embodiments, the serotonin receptor modulator is quetiapine and the psychedelic is mescaline, wherein the quetiapine is administered to post-treat at least 180 minutes post to the mescaline. In some embodiments, the serotonin receptor modulator is quetiapine and the psychedelic is mescaline, wherein the quetiapine is administered to post-treat at least 210 minutes post to the mescaline. In some embodiments, the serotonin receptor modulator is quetiapine and the psychedelic is mescaline, wherein the quetiapine is administered to post-treat at least 240 minutes post to the mescaline. In some embodiments, the serotonin receptor modulator is quetiapine and the psychedelic is mescaline, wherein the quetiapine is administered to post-treat at least 270 minutes post to the mescaline. In some embodiments, the serotonin receptor modulator is quetiapine and the psychedelic is mescaline, wherein the quetiapine is administered to post-treat at least 300 minutes post to the mescaline. In some embodiments, the serotonin receptor modulator is quetiapine and the psychedelic is mescaline, wherein the quetiapine is administered to post-treat at least 330 minutes post to the mescaline. In some embodiments, the serotonin receptor modulator is quetiapine and the psychedelic is mescaline, wherein the quetiapine is administered to post-treat at least 360 minutes post to the mescaline. In some preferred embodiments, the serotonin receptor modulator is quetiapine and the psychedelic is mescaline, wherein quetiapine is administered to post-treat between about 60 minutes and about 180 minutes post to the administration of mescaline.

In some embodiments, the serotonin receptor modulator is risperidone and the psychedelic is mescaline, wherein risperidone is administered to post-treat at least 15 minutes post to the administration of mescaline. In some embodiments, the serotonin receptor modulator is risperidone and the psychedelic is mescaline, wherein the risperidone is administered to post-treat at least 30 minutes post to the mescaline. In some embodiments, the serotonin receptor modulator is risperidone and the psychedelic is mescaline, wherein the risperidone is administered to post-treat at least 60 minutes post to the mescaline. In some embodiments, the serotonin receptor modulator is risperidone and the psychedelic is mescaline, wherein the risperidone is administered to post-treat at least 90 minutes post to the mescaline. In some embodiments, the serotonin receptor modulator is risperidone and the psychedelic is mescaline, wherein the risperidone is administered to post-treat at least 120 minutes post to the mescaline. In some embodiments, the serotonin receptor modulator is risperidone and the psychedelic is mescaline, wherein the risperidone is administered to post-treat at least 150 minutes post to the mescaline. In some embodiments, the serotonin receptor modulator is risperidone and the psychedelic is mescaline, wherein the risperidone is administered to post-treat between about 15 minutes and about 150 minutes post to the mescaline. In some embodiments, the serotonin receptor modulator is risperidone and the psychedelic is mescaline, wherein the risperidone is administered to post-treat at least 180 minutes post to the mescaline. In some embodiments, the serotonin receptor modulator is risperidone and the psychedelic is mescaline, wherein the risperidone is administered to post-treat at least 210 minutes post to the mescaline. In some embodiments, the serotonin receptor modulator is risperidone and the psychedelic is mescaline, wherein the risperidone is administered to post-treat at least 240 minutes post to the mescaline. In some embodiments, the serotonin receptor modulator is risperidone and the psychedelic is mescaline, wherein the risperidone is administered to post-treat at least 270 minutes post to the mescaline. In some embodiments, the serotonin receptor modulator is risperidone and the psychedelic is mescaline, wherein the risperidone is administered to post-treat at least 300 minutes post to the mescaline. In some embodiments, the serotonin receptor modulator is risperidone and the psychedelic is mescaline, wherein the risperidone is administered to post-treat at least 330 minutes post to the mescaline. In some embodiments, the serotonin receptor modulator is risperidone and the psychedelic is mescaline, wherein the risperidone is administered to post-treat at least 360 minutes post to the mescaline. In some preferred embodiments, the serotonin receptor modulator is risperidone and the psychedelic is mescaline, wherein risperidone is administered to post-treat between about 60 minutes and about 180 minutes post to the administration of mescaline.

In some embodiments, the serotonin receptor modulator is eplivanserin and the psychedelic is (R)-MDMA, wherein the eplivanserin is administered to post-treat at least 15 minutes post to the administration of (R)-MDMA. In some embodiments, the serotonin receptor modulator is eplivanserin and the psychedelic is (R)-MDMA, wherein the eplivanserin is administered to post-treat at least 30 minutes post to the (R)-MDMA. In some embodiments, the serotonin receptor modulator is eplivanserin and the psychedelic is (R)-MDMA, wherein the eplivanserin is administered to post-treat at least 60 minutes post to the (R)-MDMA. In some embodiments, the serotonin receptor modulator is eplivanserin and the psychedelic is (R)-MDMA, wherein the eplivanserin is administered to post-treat at least 90 minutes post to the (R)-MDMA. In some embodiments, the serotonin receptor modulator is eplivanserin and the psychedelic is (R)-MDMA, wherein the eplivanserin is administered to post-treat between at least 30 minutes post and 360 minutes post to the release or administration of the (R)-MDMA. In some embodiments, the serotonin receptor modulator is eplivanserin and the psychedelic is (R)-MDMA, wherein the eplivanserin is administered to post-treat between at least 60 minutes post and 360 minutes post to the release or administration the (R)-MDMA. In some embodiments, the serotonin receptor modulator is eplivanserin and the psychedelic is (R)-MDMA, wherein the eplivanserin is administered to post-treat between at least 90 minutes and 240 minutes post to the (R)-MDMA. In some embodiments, the serotonin receptor modulator is eplivanserin and the psychedelic is (R)-MDMA, wherein the eplivanserin is administered to post-treat at least 120 minutes post to the (R)-MDMA. In some embodiments, the serotonin receptor modulator is eplivanserin and the psychedelic is (R)-MDMA, wherein the eplivanserin is administered to post-treat at least 150 minutes post to the (R)-MDMA. In some embodiments, the serotonin receptor modulator is eplivanserin and the psychedelic is (R)-MDMA, wherein the eplivanserin is administered to post-treat between about 15 minutes and about 150 minutes post to the (R)-MDMA. In some embodiments, the serotonin receptor modulator is eplivanserin and the psychedelic is (R)-MDMA, wherein the eplivanserin is administered to post-treat at least 180 minutes post to the (R)-MDMA. In some embodiments, the serotonin receptor modulator is eplivanserin and the psychedelic is (R)-MDMA, wherein the eplivanserin is administered to post-treat at least 210 minutes post to the (R)-MDMA. In some embodiments, the serotonin receptor modulator is eplivanserin and the psychedelic is (R)-MDMA, wherein the eplivanserin is administered to post-treat at least 240 minutes post to the (R)-MDMA.

In some embodiments, the serotonin receptor modulator is eplivanserin and the psychedelic is (R)-MDMA, wherein the eplivanserin is administered to post-treat at least 270 minutes post to the (R)-MDMA. In some embodiments, the serotonin receptor modulator is eplivanserin and the psychedelic is (R)-MDMA, wherein the eplivanserin is administered to post-treat at least 300 minutes post to the (R)-MDMA. In some embodiments, the serotonin receptor modulator is eplivanserin and the psychedelic is (R)-MDMA, wherein the eplivanserin is administered to post-treat at least 330 minutes post to the (R)-MDMA. In some embodiments, the serotonin receptor modulator is eplivanserin and the psychedelic is (R)-MDMA, wherein the eplivanserin is administered to post-treat at least 360 minutes post to the (R)-MDMA.

In some preferred embodiments, the serotonin receptor modulator is eplivanserin and the psychedelic is (R)-MDMA, wherein eplivanserin is administered to post-treat between about 60 minutes and about 180 minutes post to the administration of (R)-MDMA.

In some embodiments, the serotonin receptor modulator is volinanserin and the psychedelic is (R)-MDMA, wherein the volinanserin is administered to post-treat at least 15 minutes post to the administration of (R)-MDMA. In some embodiments, the serotonin receptor modulator is volinanserin and the psychedelic is (R)-MDMA, wherein the volinanserin is administered to post-treat at least 30 minutes post to the (R)-MDMA. In some embodiments, the serotonin receptor modulator is volinanserin and the psychedelic is (R)-MDMA, wherein the volinanserin is administered to post-treat at least 60 minutes post to the (R)-MDMA. In some embodiments, the serotonin receptor modulator is volinanserin and the psychedelic is (R)-MDMA, wherein the volinanserin is administered to post-treat a subject between at least 15 minutes and 360 minutes post to the administration or release of (R)-MDMA. In some embodiments, the serotonin receptor modulator is volinanserin and the psychedelic is (R)-MDMA, wherein the volinanserin is administered to post-treat between at least 30 minutes and 360 minutes post to the administration or release of (R)-MDMA. In some embodiments, the serotonin receptor modulator is volinanserin and the psychedelic is (R)-MDMA, wherein the volinanserin is administered to post-treat between at least 60 minutes and 240 minutes post to the administration or release of (R)-MDMA. In some embodiments, the serotonin receptor modulator is volinanserin and the psychedelic is (R)-MDMA, wherein the volinanserin is administered to post-treat at least 90 minutes post to the (R)-MDMA. In some embodiments, the serotonin receptor modulator is volinanserin and the psychedelic is (R)-MDMA, wherein the volinanserin is administered to post-treat at least 120 minutes post to the (R)-MDMA.

In some embodiments, the serotonin receptor modulator is volinanserin and the psychedelic is (R)-MDMA, wherein the volinanserin is administered to post-treat at least 150 minutes post to the (R)-MDMA. In some embodiments, the serotonin receptor modulator is volinanserin and the psychedelic is (R)-MDMA, wherein the volinanserin is administered to post-treat between about 15 minutes and about 150 minutes post to the (R)-MDMA. In some embodiments, the serotonin receptor modulator is volinanserin and the psychedelic is (R)-MDMA, wherein the volinanserin is administered to post-treat at least 180 minutes post to the (R)-MDMA. In some embodiments, the serotonin receptor modulator is volinanserin and the psychedelic is (R)-MDMA, wherein the volinanserin is administered to post-treat at least 210 minutes post to the (R)-MDMA. In some embodiments, the serotonin receptor modulator is volinanserin and the psychedelic is (R)-MDMA, wherein the volinanserin is administered to post-treat at least 240 minutes post to the (R)-MDMA. In some embodiments, the serotonin receptor modulator is volinanserin and the psychedelic is (R)-MDMA, wherein the volinanserin is administered to post-treat at least 270 minutes post to the (R)-MDMA. In some embodiments, the serotonin receptor modulator is volinanserin and the psychedelic is (R)-MDMA, wherein the volinanserin is administered to post-treat at least 300 minutes post to the (R)-MDMA. In some embodiments, the serotonin receptor modulator is volinanserin and the psychedelic is (R)-MDMA, wherein the volinanserin is administered to post-treat at least 330 minutes post to the (R)-MDMA. In some embodiments, the serotonin receptor modulator is volinanserin and the psychedelic is (R)-MDMA, wherein the volinanserin is administered to post-treat at least 360 minutes post to the (R)-MDMA. In some preferred embodiments, the serotonin receptor modulator is volinanserin and the psychedelic is (R)-MDMA, wherein volinanserin is administered to post-treat between about 60 minutes and about 180 minutes post to the administration of (R)-MDMA.

In some embodiments, the serotonin receptor modulator is ketanserin and the psychedelic is (R)-MDMA, wherein the ketanserin is administered to post-treat at least 15 minutes post to the administration of (R)-MDMA. In some embodiments, the serotonin receptor modulator is ketanserin and the psychedelic is (R)-MDMA, wherein the ketanserin is administered to post-treat at least 30 minutes post to the (R)-MDMA. In some embodiments, the serotonin receptor modulator is ketanserin and the psychedelic is (R)-MDMA, wherein the ketanserin is administered to post-treat at least 60 minutes post to the (R)-MDMA. In some embodiments, the serotonin receptor modulator is ketanserin and the psychedelic is (R)-MDMA, wherein the ketanserin is administered to post-treat between at least 30 minutes and 360 minutes post to the administration or release (R)-MDMA. In some embodiments, the serotonin receptor modulator is ketanserin and the psychedelic is (R)-MDMA, wherein the ketanserin is administered to post-treat between at least 60 minutes and 240 minutes post to the administration or release of (R)-MDMA. In some embodiments, the serotonin receptor modulator is ketanserin and the psychedelic is (R)-MDMA, wherein the ketanserin is administered to post-treat at least 90 minutes post to the (R)-MDMA. In some embodiments, the serotonin receptor modulator is ketanserin and the psychedelic is (R)-MDMA, wherein the ketanserin is administered to post-treat at least 120 minutes post to the (R)-MDMA. In some embodiments, the serotonin receptor modulator is ketanserin and the psychedelic is (R)-MDMA, wherein the ketanserin is administered to post-treat at least 150 minutes post to the (R)-MDMA. In some embodiments, the serotonin receptor modulator is ketanserin and the psychedelic is (R)-MDMA, wherein the ketanserin is administered to post-treat between about 15 minutes and about 150 minutes post to the (R)-MDMA. In some embodiments, the serotonin receptor modulator is ketanserin and the psychedelic is (R)-MDMA, wherein the ketanserin is administered to post-treat at least 180 minutes post to the (R)-MDMA. In some embodiments, the serotonin receptor modulator is ketanserin and the psychedelic is (R)-MDMA, wherein the ketanserin is administered to post-treat at least 210 minutes post to the (R)-MDMA. In some embodiments, the serotonin receptor modulator is ketanserin and the psychedelic is (R)-MDMA, wherein the ketanserin is administered to post-treat at least 240 minutes post to the (R)-MDMA. In some embodiments, the serotonin receptor modulator is ketanserin and the psychedelic is (R)-MDMA, wherein the ketanserin is administered to post-treat at least 270 minutes post to the (R)-MDMA. In some embodiments, the serotonin receptor modulator is ketanserin and the psychedelic is (R)-MDMA, wherein the ketanserin is administered to post-treat at least 300 minutes post to the (R)-MDMA. In some embodiments, the serotonin receptor modulator is ketanserin and the psychedelic is (R)-MDMA, wherein the ketanserin is administered to post-treat at least 330 minutes post to the (R)-MDMA. In some embodiments, the serotonin receptor modulator is ketanserin and the psychedelic is (R)-MDMA, wherein the ketanserin is administered to post-treat at least 360 minutes post to the (R)-MDMA. In some preferred embodiments, the serotonin receptor modulator is ketanserin and the psychedelic is (R)-MDMA, wherein ketanserin is administered to post-treat between about 60 minutes and about 180 minutes post to the administration of (R)-MDMA.

In some embodiments, the serotonin receptor modulator is ritanserin and the psychedelic is (R)-MDMA, wherein the ritanserin is administered to post-treat at least 15 minutes post to the administration of (R)-MDMA. In some embodiments, the serotonin receptor modulator is ritanserin and the psychedelic is (R)-MDMA, wherein the ritanserin is administered to post-treat at least 30 minutes post to the (R)-MDMA. In some embodiments, the serotonin receptor modulator is ritanserin and the psychedelic is (R)-MDMA, wherein the ritanserin is administered to post-treat at least 60 minutes post to the (R)-MDMA. In some embodiments, the serotonin receptor modulator is ritanserin and the psychedelic is (R)-MDMA, wherein the ritanserin is administered to post-treat between at least 60 minutes and 240 minutes post to the administration or release of (R)-MDMA. In some embodiments, the serotonin receptor modulator is ritanserin and the psychedelic is (R)-MDMA, wherein the ritanserin is administered to post-treat at least 90 minutes post to the (R)-MDMA. In some embodiments, the serotonin receptor modulator is ritanserin and the psychedelic is (R)-MDMA, wherein the ritanserin is administered to post-treat at least 120 minutes post to the (R)-MDMA. In some embodiments, the serotonin receptor modulator is ritanserin and the psychedelic is (R)-MDMA, wherein the ritanserin is administered to post-treat at least 150 minutes post to the (R)-MDMA. In some embodiments, the serotonin receptor modulator is ritanserin and the psychedelic is (R)-MDMA, wherein the ritanserin is administered to post-treat between about 15 minutes and about 150 minutes post to the (R)-MDMA. In some embodiments, the serotonin receptor modulator is ritanserin and the psychedelic is (R)-MDMA, wherein the ritanserin is administered to post-treat at least 180 minutes post to the (R)-MDMA. In some embodiments, the serotonin receptor modulator is ritanserin and the psychedelic is (R)-MDMA, wherein the ritanserin is administered to post-treat at least 210 minutes post to the (R)-MDMA. In some embodiments, the serotonin receptor modulator is ritanserin and the psychedelic is (R)-MDMA, wherein the ritanserin is administered to post-treat at least 240 minutes post to the (R)-MDMA. In some embodiments, the serotonin receptor modulator is ritanserin and the psychedelic is (R)-MDMA, wherein the ritanserin is administered to post-treat at least 270 minutes post to the (R)-MDMA. In some embodiments, the serotonin receptor modulator is ritanserin and the psychedelic is (R)-MDMA, wherein the ritanserin is administered to post-treat at least 300 minutes post to the (R)-MDMA. In some embodiments, the serotonin receptor modulator is ritanserin and the psychedelic is (R)-MDMA, wherein the ritanserin is administered to post-treat at least 330 minutes post to the (R)-MDMA. In some embodiments, the serotonin receptor modulator is ritanserin and the psychedelic is (R)-MDMA, wherein the ritanserin is administered to post-treat at least 360 minutes post to the (R)-MDMA. In some preferred embodiments, the serotonin receptor modulator is ritanserin and the psychedelic is (R)-MDMA, wherein ritanserin is administered to post-treat between about 60 minutes and about 180 minutes post to the administration of (R)-MDMA.

In some embodiments, the serotonin receptor modulator is pimavanserin and the psychedelic is (R)-MDMA, wherein the pimavanserin is administered to post-treat at least 15 minutes post to the administration of (R)-MDMA. In some embodiments, the serotonin receptor modulator is pimavanserin and the psychedelic is (R)-MDMA, wherein the pimavanserin is administered to post-treat at least 30 minutes post to the (R)-MDMA. In some embodiments, the serotonin receptor modulator is pimavanserin and the psychedelic is (R)-MDMA, wherein the pimavanserin is administered to post-treat at least 60 minutes post to the (R)-MDMA. In some embodiments, the serotonin receptor modulator is pimavanserin and the psychedelic is (R)-MDMA, wherein the pimavanserin is administered to post-treat between at least 60 minutes and 240 minutes post to the administration or release of (R)-MDMA. In some embodiments, the serotonin receptor modulator is pimavanserin and the psychedelic is (R)-MDMA, wherein the pimavanserin is administered to post-treat at least 90 minutes post to the (R)-MDMA. In some embodiments, the serotonin receptor modulator is pimavanserin and the psychedelic is (R)-MDMA, wherein the pimavanserin is administered to post-treat at least 120 minutes post to the (R)-MDMA. In some embodiments, the serotonin receptor modulator is pimavanserin and the psychedelic is (R)-MDMA, wherein the pimavanserin is administered to post-treat at least 150 minutes post to the (R)-MDMA. In some embodiments, the serotonin receptor modulator is pimavanserin and the psychedelic is (R)-MDMA, wherein the pimavanserin is administered to post-treat between about 15 minutes and about 150 minutes post to the (R)-MDMA. In some embodiments, the serotonin receptor modulator is pimavanserin and the psychedelic is (R)-MDMA, wherein the pimavanserin is administered to post-treat at least 180 minutes post to the (R)-MDMA. In some embodiments, the serotonin receptor modulator is pimavanserin and the psychedelic is (R)-MDMA, wherein the pimavanserin is administered to post-treat at least 210 minutes post to the (R)-MDMA. In some embodiments, the serotonin receptor modulator is pimavanserin and the psychedelic is (R)-MDMA, wherein the pimavanserin is administered to post-treat at least 240 minutes post to the (R)-MDMA. In some embodiments, the serotonin receptor modulator is pimavanserin and the psychedelic is (R)-MDMA, wherein the pimavanserin is administered to post-treat at least 270 minutes post to the (R)-MDMA. In some embodiments, the serotonin receptor modulator is pimavanserin and the psychedelic is (R)-MDMA, wherein the pimavanserin is administered to post-treat at least 300 minutes post to the (R)-MDMA. In some embodiments, the serotonin receptor modulator is pimavanserin and the psychedelic is (R)-MDMA, wherein the pimavanserin is administered to post-treat at least 330 minutes post to the (R)-MDMA. In some embodiments, the serotonin receptor modulator is pimavanserin and the psychedelic is (R)-MDMA, wherein the pimavanserin is administered to post-treat at least 360 minutes post to the (R)-MDMA. In some preferred embodiments, the serotonin receptor modulator is pimavanserin and the psychedelic is (R)-MDMA, wherein pimavanserin is administered to post-treat between about 60 minutes and about 180 minutes post to the administration of (R)-MDMA.

In some embodiments, the serotonin receptor modulator is nelotanserin and the psychedelic is (R)-MDMA, wherein the nelotanserin is administered to post-treat at least 15 minutes post to the administration of (R)-MDMA. In some embodiments, the serotonin receptor modulator is nelotanserin and the psychedelic is (R)-MDMA, wherein the nelotanserin is administered to post-treat at least 30 minutes post to the (R)-MDMA. In some embodiments, the serotonin receptor modulator is nelotanserin and the psychedelic is (R)-MDMA, wherein the nelotanserin is administered to post-treat at least 60 minutes post to the (R)-MDMA. In some embodiments, the serotonin receptor modulator is nelotanserin and the psychedelic is (R)-MDMA, wherein the nelotanserin is administered to post-treat between at least 60 minutes and 240 minutes post to the administration or release of (R)-MDMA. In some embodiments, the serotonin receptor modulator is nelotanserin and the psychedelic is (R)-MDMA, wherein the nelotanserin is administered to post-treat at least 90 minutes post to the (R)-MDMA. In some embodiments, the serotonin receptor modulator is nelotanserin and the psychedelic is (R)-MDMA, wherein the nelotanserin is administered to post-treat at least 120 minutes post to the (R)-MDMA.

In some embodiments, the serotonin receptor modulator is nelotanserin and the psychedelic is (R)-MDMA, wherein the nelotanserin is administered to post-treat at least 150 minutes post to the (R)-MDMA. In some embodiments, the serotonin receptor modulator is nelotanserin and the psychedelic is (R)-MDMA, wherein the nelotanserin is administered to post-treat between about 15 minutes and about 150 minutes post to the (R)-MDMA. In some embodiments, the serotonin receptor modulator is nelotanserin and the psychedelic is (R)-MDMA, wherein the nelotanserin is administered to post-treat at least 180 minutes post to the (R)-MDMA. In some embodiments, the serotonin receptor modulator is nelotanserin and the psychedelic is (R)-MDMA, wherein the nelotanserin is administered to post-treat at least 210 minutes post to the (R)-MDMA. In some embodiments, the serotonin receptor modulator is nelotanserin and the psychedelic is (R)-MDMA, wherein the nelotanserin is administered to post-treat at least 240 minutes post to the (R)-MDMA. In some embodiments, the serotonin receptor modulator is nelotanserin and the psychedelic is (R)-MDMA, wherein the nelotanserin is administered to post-treat at least 270 minutes post to the (R)-MDMA.

In some embodiments, the serotonin receptor modulator is nelotanserin and the psychedelic is (R)-MDMA, wherein the nelotanserin is administered to post-treat at least 300 minutes post to the (R)-MDMA. In some embodiments, the serotonin receptor modulator is nelotanserin and the psychedelic is (R)-MDMA, wherein the nelotanserin is administered to post-treat at least 330 minutes post to the (R)-MDMA. In some embodiments, the serotonin receptor modulator is nelotanserin and the psychedelic is (R)-MDMA, wherein the nelotanserin is administered to post-treat at least 360 minutes post to the (R)-MDMA. In some preferred embodiments, the serotonin receptor modulator is nelotanserin and the psychedelic is (R)-MDMA, wherein nelotanserin is administered to post-treat between about 60 minutes and about 180 minutes post to the administration of (R)-MDMA.

In some embodiments, the serotonin receptor modulator is pruvanserin and the psychedelic is (R)-MDMA, wherein the pruvanserin is administered to post-treat at least 15 minutes post to the administration of (R)-MDMA. In some embodiments, the serotonin receptor modulator is pruvanserin and the psychedelic is (R)-MDMA, wherein the pruvanserin is administered to post-treat at least 30 minutes post to the (R)-MDMA. In some embodiments, the serotonin receptor modulator is pruvanserin and the psychedelic is (R)-MDMA, wherein the pruvanserin is administered to post-treat at least 60 minutes post to the (R)-MDMA. In some embodiments, the serotonin receptor modulator is pruvanserin and the psychedelic is (R)-MDMA, wherein the pruvanserin is administered to post-treat between at least 60 minutes and 240 minutes post to the administration or release of (R)-MDMA. In some embodiments, the serotonin receptor modulator is pruvanserin and the psychedelic is (R)-MDMA, wherein the pruvanserin is administered to post-treat at least 90 minutes post to the (R)-MDMA. In some embodiments, the serotonin receptor modulator is pruvanserin and the psychedelic is (R)-MDMA, wherein the pruvanserin is administered to post-treat at least 120 minutes post to the (R)-MDMA. In some embodiments, the serotonin receptor modulator is pruvanserin and the psychedelic is (R)-MDMA, wherein the pruvanserin is administered to post-treat at least 150 minutes post to the (R)-MDMA. In some embodiments, the serotonin receptor modulator is pruvanserin and the psychedelic is (R)-MDMA, wherein the pruvanserin is administered to post-treat between about 15 minutes and about 150 minutes post to the (R)-MDMA. In some embodiments, the serotonin receptor modulator is pruvanserin and the psychedelic is (R)-MDMA, wherein the pruvanserin is administered to post-treat at least 180 minutes post to the (R)-MDMA. In some embodiments, the serotonin receptor modulator is pruvanserin and the psychedelic is (R)-MDMA, wherein the pruvanserin is administered to post-treat at least 210 minutes post to the (R)-MDMA.

In some embodiments, the serotonin receptor modulator is pruvanserin and the psychedelic is (R)-MDMA, wherein the pruvanserin is administered to post-treat at least 240 minutes post to the (R)-MDMA. In some embodiments, the serotonin receptor modulator is pruvanserin and the psychedelic is (R)-MDMA, wherein the pruvanserin is administered to post-treat at least 270 minutes post to the (R)-MDMA. In some embodiments, the serotonin receptor modulator is pruvanserin and the psychedelic is (R)-MDMA, wherein the pruvanserin is administered to post-treat at least 300 minutes post to the (R)-MDMA. In some embodiments, the serotonin receptor modulator is pruvanserin and the psychedelic is (R)-MDMA, wherein the pruvanserin is administered to post-treat at least 330 minutes post to the (R)-MDMA. In some embodiments, the serotonin receptor modulator is pruvanserin and the psychedelic is (R)-MDMA, wherein the pruvanserin is administered to post-treat at least 360 minutes post to the (R)-MDMA. In some preferred embodiments, the serotonin receptor modulator is pruvanserin and the psychedelic is (R)-MDMA, wherein pruvanserin is administered to post-treat between about 60 minutes and about 180 minutes post to the administration of (R)-MDMA.

In some embodiments, the serotonin receptor modulator is flibanserin and the psychedelic is (R)-MDMA, wherein the flibanserin is administered to post-treat at least 15 minutes post to the administration of (R)-MDMA. In some embodiments, the serotonin receptor modulator is flibanserin and the psychedelic is (R)-MDMA, wherein the flibanserin is administered to post-treat at least 30 minutes post to the (R)-MDMA. In some embodiments, the serotonin receptor modulator is flibanserin and the psychedelic is (R)-MDMA, wherein the flibanserin is administered to post-treat at least 60 minutes post to the (R)-MDMA. In some embodiments, the serotonin receptor modulator is flibanserin and the psychedelic is (R)-MDMA, wherein the flibanserin is administered to post-treat at least 90 minutes post to the (R)-MDMA. In some embodiments, the serotonin receptor modulator is flibanserin and the psychedelic is (R)-MDMA, wherein the flibanserin is administered to post-treat at least 120 minutes post to the (R)-MDMA. In some embodiments, the serotonin receptor modulator is flibanserin and the psychedelic is (R)-MDMA, wherein the flibanserin is administered to post-treat at least 150 minutes post to the (R)-MDMA. In some embodiments, the serotonin receptor modulator is flibanserin and the psychedelic is (R)-MDMA, wherein the flibanserin is administered to post-treat between about 15 minutes and about 150 minutes post to the (R)-MDMA. In some embodiments, the serotonin receptor modulator is flibanserin and the psychedelic is (R)-MDMA, wherein the flibanserin is administered to post-treat at least 180 minutes post to the (R)-MDMA. In some embodiments, the serotonin receptor modulator is flibanserin and the psychedelic is (R)-MDMA, wherein the flibanserin is administered to post-treat at least 210 minutes post to the (R)-MDMA. In some embodiments, the serotonin receptor modulator is flibanserin and the psychedelic is (R)-MDMA, wherein the flibanserin is administered to post-treat at least 240 minutes post to the (R)-MDMA. In some embodiments, the serotonin receptor modulator is flibanserin and the psychedelic is (R)-MDMA, wherein the flibanserin is administered to post-treat at least 270 minutes post to the (R)-MDMA. In some embodiments, the serotonin receptor modulator is flibanserin and the psychedelic is (R)-MDMA, wherein the flibanserin is administered to post-treat at least 300 minutes post to the (R)-MDMA. In some embodiments, the serotonin receptor modulator is flibanserin and the psychedelic is (R)-MDMA, wherein the flibanserin is administered to post-treat at least 330 minutes post to the (R)-MDMA. In some embodiments, the serotonin receptor modulator is flibanserin and the psychedelic is (R)-MDMA, wherein the flibanserin is administered to post-treat at least 360 minutes post to the (R)-MDMA. In some preferred embodiments, the serotonin receptor modulator is flibanserin and the psychedelic is (R)-MDMA, wherein flibanserin is administered to post-treat between about 60 minutes and about 180 minutes post to the administration of (R)-MDMA.

In some embodiments, the serotonin receptor modulator is olanzapine and the psychedelic is (R)-MDMA, wherein the olanzapine is administered to post-treat at least 15 minutes post to the administration of (R)-MDMA. In some embodiments, the serotonin receptor modulator is olanzapine and the psychedelic is (R)-MDMA, wherein the olanzapine is administered to post-treat at least 30 minutes post to the (R)-MDMA. In some embodiments, the serotonin receptor modulator is olanzapine and the psychedelic is (R)-MDMA, wherein the olanzapine is administered to post-treat at least 60 minutes post to the (R)-MDMA. In some embodiments, the serotonin receptor modulator is olanzapine and the psychedelic is (R)-MDMA, wherein the olanzapine is administered to post-treat at least 90 minutes post to the (R)-MDMA. In some embodiments, the serotonin receptor modulator is olanzapine and the psychedelic is (R)-MDMA, wherein the olanzapine is administered to post-treat at least 120 minutes post to the (R)-MDMA. In some embodiments, the serotonin receptor modulator is olanzapine and the psychedelic is (R)-MDMA, wherein the olanzapine is administered to post-treat at least 150 minutes post to the (R)-MDMA. In some embodiments, the serotonin receptor modulator is olanzapine and the psychedelic is (R)-MDMA, wherein the olanzapine is administered to post-treat between about 15 minutes and about 150 minutes post to the (R)-MDMA. In some embodiments, the serotonin receptor modulator is olanzapine and the psychedelic is (R)-MDMA, wherein the olanzapine is administered to post-treat at least 180 minutes post to the (R)-MDMA. In some embodiments, the serotonin receptor modulator is olanzapine and the psychedelic is (R)-MDMA, wherein the olanzapine is administered to post-treat at least 210 minutes post to the (R)-MDMA. In some embodiments, the serotonin receptor modulator is olanzapine and the psychedelic is (R)-MDMA, wherein the olanzapine is administered to post-treat at least 240 minutes post to the (R)-MDMA. In some embodiments, the serotonin receptor modulator is olanzapine and the psychedelic is (R)-MDMA, wherein the olanzapine is administered to post-treat at least 270 minutes post to the (R)-MDMA. In some embodiments, the serotonin receptor modulator is olanzapine and the psychedelic is (R)-MDMA, wherein the olanzapine is administered to post-treat at least 300 minutes post to the (R)-MDMA. In some embodiments, the serotonin receptor modulator is olanzapine and the psychedelic is (R)-MDMA, wherein the olanzapine is administered to post-treat at least 330 minutes post to the (R)-MDMA. In some embodiments, the serotonin receptor modulator is olanzapine and the psychedelic is (R)-MDMA, wherein the olanzapine is administered to post-treat at least 360 minutes post to the (R)-MDMA. In some preferred embodiments, the serotonin receptor modulator is olanzapine and the psychedelic is (R)-MDMA, wherein olanzapine is administered to post-treat between about 60 minutes and about 180 minutes post to the administration of (R)-MDMA.

In some embodiments, the serotonin receptor modulator is quetiapine and the psychedelic is (R)-MDMA, wherein the quetiapine is administered to post-treat at least 15 minutes post to the administration of (R)-MDMA. In some embodiments, the serotonin receptor modulator is quetiapine and the psychedelic is (R)-MDMA, wherein the quetiapine is administered to post-treat at least 30 minutes post to the (R)-MDMA. In some embodiments, the serotonin receptor modulator is quetiapine and the psychedelic is (R)-MDMA, wherein the quetiapine is administered to post-treat at least 60 minutes post to the (R)-MDMA. In some embodiments, the serotonin receptor modulator is quetiapine and the psychedelic is (R)-MDMA, wherein the quetiapine is administered to post-treat at least 90 minutes post to the (R)-MDMA. In some embodiments, the serotonin receptor modulator is quetiapine and the psychedelic is (R)-MDMA, wherein the quetiapine is administered to post-treat at least 120 minutes post to the (R)-MDMA. In some embodiments, the serotonin receptor modulator is quetiapine and the psychedelic is (R)-MDMA, wherein the quetiapine is administered to post-treat at least 150 minutes post to the (R)-MDMA. In some embodiments, the serotonin receptor modulator is quetiapine and the psychedelic is (R)-MDMA, wherein the quetiapine is administered to post-treat between about 15 minutes and about 150 minutes post to the (R)-MDMA. In some embodiments, the serotonin receptor modulator is quetiapine and the psychedelic is (R)-MDMA, wherein the quetiapine is administered to post-treat at least 180 minutes post to the (R)-MDMA. In some embodiments, the serotonin receptor modulator is quetiapine and the psychedelic is (R)-MDMA, wherein the quetiapine is administered to post-treat at least 210 minutes post to the (R)-MDMA. In some embodiments, the serotonin receptor modulator is quetiapine and the psychedelic is (R)-MDMA, wherein the quetiapine is administered to post-treat at least 240 minutes post to the (R)-MDMA. In some embodiments, the serotonin receptor modulator is quetiapine and the psychedelic is (R)-MDMA, wherein the quetiapine is administered to post-treat at least 270 minutes post to the (R)-MDMA. In some embodiments, the serotonin receptor modulator is quetiapine and the psychedelic is (R)-MDMA, wherein the quetiapine is administered to post-treat at least 300 minutes post to the (R)-MDMA. In some embodiments, the serotonin receptor modulator is quetiapine and the psychedelic is (R)-MDMA, wherein the quetiapine is administered to post-treat at least 330 minutes post to the (R)-MDMA. In some embodiments, the serotonin receptor modulator is quetiapine and the psychedelic is (R)-MDMA, wherein the quetiapine is administered to post-treat at least 360 minutes post to the (R)-MDMA. In some preferred embodiments, the serotonin receptor modulator is quetiapine and the psychedelic is (R)-MDMA, wherein quetiapine is administered to post-treat between about 60 minutes and about 180 minutes post to the administration of (R)-MDMA.

In some embodiments, the serotonin receptor modulator is risperidone and the psychedelic is (R)-MDMA, wherein the risperidone is administered to post-treat at least 15 minutes post to the administration of (R)-MDMA. In some embodiments, the serotonin receptor modulator is risperidone and the psychedelic is (R)-MDMA, wherein the risperidone is administered to post-treat at least 30 minutes post to the (R)-MDMA. In some embodiments, the serotonin receptor modulator is risperidone and the psychedelic is (R)-MDMA, wherein the risperidone is administered to post-treat at least 60 minutes post to the (R)-MDMA. In some embodiments, the serotonin receptor modulator is risperidone and the psychedelic is (R)-MDMA, wherein the risperidone is administered to post-treat at least 90 minutes post to the (R)-MDMA. In some embodiments, the serotonin receptor modulator is risperidone and the psychedelic is (R)-MDMA, wherein the risperidone is administered to post-treat at least 120 minutes post to the (R)-MDMA. In some embodiments, the serotonin receptor modulator is risperidone and the psychedelic is (R)-MDMA, wherein the risperidone is administered to post-treat at least 150 minutes post to the (R)-MDMA. In some embodiments, the serotonin receptor modulator is risperidone and the psychedelic is (R)-MDMA, wherein the risperidone is administered to post-treat between about 15 minutes and about 150 minutes post to the (R)-MDMA. In some embodiments, the serotonin receptor modulator is risperidone and the psychedelic is (R)-MDMA, wherein the risperidone is administered to post-treat at least 180 minutes post to the (R)-MDMA. In some embodiments, the serotonin receptor modulator is risperidone and the psychedelic is (R)-MDMA, wherein the risperidone is administered to post-treat at least 210 minutes post to the (R)-MDMA. In some embodiments, the serotonin receptor modulator is risperidone and the psychedelic is (R)-MDMA, wherein the risperidone is administered to post-treat at least 240 minutes post to the (R)-MDMA. In some embodiments, the serotonin receptor modulator is risperidone and the psychedelic is (R)-MDMA, wherein the risperidone is administered to post-treat at least 270 minutes post to the (R)-MDMA. In some embodiments, the serotonin receptor modulator is risperidone and the psychedelic is (R)-MDMA, wherein the risperidone is administered to post-treat at least 300 minutes post to the (R)-MDMA. In some embodiments, the serotonin receptor modulator is risperidone and the psychedelic is (R)-MDMA, wherein the risperidone is administered to post-treat at least 330 minutes post to the (R)-MDMA. In some embodiments, the serotonin receptor modulator is risperidone and the psychedelic is (R)-MDMA, wherein the risperidone is administered to post-treat at least 360 minutes post to the (R)-MDMA. In some preferred embodiments, the serotonin receptor modulator is risperidone and the psychedelic is (R)-MDMA, wherein risperidone is administered to post-treat between about 60 minutes and about 180 minutes post to the administration of (R)-MDMA.

In some embodiments, the serotonin receptor modulator is eplivanserin and the psychedelic is (S)-MDMA, wherein the eplivanserin is administered to post-treat at least 15 minutes post to the administration of (S)-MDMA. In some embodiments, the serotonin receptor modulator is eplivanserin and the psychedelic is (S)-MDMA, wherein the eplivanserin is administered to post-treat at least 30 minutes post to the (S)-MDMA. In some embodiments, the serotonin receptor modulator is eplivanserin and the psychedelic is (S)-MDMA, wherein the eplivanserin is administered to post-treat at least 60 minutes post to the (S)-MDMA. In some embodiments, the serotonin receptor modulator is eplivanserin and the psychedelic is (S)-MDMA, wherein the eplivanserin is administered to post-treat at least 90 minutes post to the (S)-MDMA. In some embodiments, the serotonin receptor modulator is eplivanserin and the psychedelic is (S)-MDMA, wherein the eplivanserin is administered to post-treat between at least 30 minutes post and 360 minutes post to the release or administration of the (S)-MDMA. In some embodiments, the serotonin receptor modulator is eplivanserin and the psychedelic is (S)-MDMA, wherein the eplivanserin is administered to post-treat between at least 60 minutes post and 360 minutes post to the release or administration the (S)-MDMA. In some embodiments, the serotonin receptor modulator is eplivanserin and the psychedelic is (S)-MDMA, wherein the eplivanserin is administered to post-treat between at least 90 minutes and 240 minutes post to the (S)-MDMA. In some embodiments, the serotonin receptor modulator is eplivanserin and the psychedelic is (S)-MDMA, wherein the eplivanserin is administered to post-treat at least 120 minutes post to the (S)-MDMA. In some embodiments, the serotonin receptor modulator is eplivanserin and the psychedelic is (S)-MDMA, wherein the eplivanserin is administered to post-treat at least 150 minutes post to the (S)-MDMA. In some embodiments, the serotonin receptor modulator is eplivanserin and the psychedelic is (S)-MDMA, wherein the eplivanserin is administered to post-treat between about 15 minutes and about 150 minutes post to the (S)-MDMA. In some embodiments, the serotonin receptor modulator is eplivanserin and the psychedelic is (S)-MDMA, wherein the eplivanserin is administered to post-treat at least 180 minutes post to the (S)-MDMA. In some embodiments, the serotonin receptor modulator is eplivanserin and the psychedelic is (S)-MDMA, wherein the eplivanserin is administered to post-treat at least 210 minutes post to the (S)-MDMA. In some embodiments, the serotonin receptor modulator is eplivanserin and the psychedelic is (S)-MDMA, wherein the eplivanserin is administered to post-treat at least 240 minutes post to the (S)-MDMA.

In some embodiments, the serotonin receptor modulator is eplivanserin and the psychedelic is (S)-MDMA, wherein the eplivanserin is administered to post-treat at least 270 minutes post to the (S)-MDMA. In some embodiments, the serotonin receptor modulator is eplivanserin and the psychedelic is (S)-MDMA, wherein the eplivanserin is administered to post-treat at least 300 minutes post to the (S)-MDMA. In some embodiments, the serotonin receptor modulator is eplivanserin and the psychedelic is (S)-MDMA, wherein the eplivanserin is administered to post-treat at least 330 minutes post to the (S)-MDMA. In some embodiments, the serotonin receptor modulator is eplivanserin and the psychedelic is (S)-MDMA, wherein the eplivanserin is administered to post-treat at least 360 minutes post to the (S)-MDMA.

In some preferred embodiments, the serotonin receptor modulator is eplivanserin and the psychedelic is (S)-MDMA, wherein eplivanserin is administered to post-treat between about 60 minutes and about 180 minutes post to the administration of (S)-MDMA.

In some embodiments, the serotonin receptor modulator is volinanserin and the psychedelic is (S)-MDMA, wherein the volinanserin is administered to post-treat at least 15 minutes post to the administration of (S)-MDMA. In some embodiments, the serotonin receptor modulator is volinanserin and the psychedelic is (S)-MDMA, wherein the volinanserin is administered to post-treat at least 30 minutes post to the (S)-MDMA. In some embodiments, the serotonin receptor modulator is volinanserin and the psychedelic is (S)-MDMA, wherein the volinanserin is administered to post-treat at least 60 minutes post to the (S)-MDMA. In some embodiments, the serotonin receptor modulator is volinanserin and the psychedelic is (S)-MDMA, wherein the volinanserin is administered to post-treat a subject between at least 15 minutes and 360 minutes post to the administration or release of (S)-MDMA. In some embodiments, the serotonin receptor modulator is volinanserin and the psychedelic is (S)-MDMA, wherein the volinanserin is administered to post-treat between at least 30 minutes and 360 minutes post to the administration or release of (S)-MDMA. In some embodiments, the serotonin receptor modulator is volinanserin and the psychedelic is (S)-MDMA, wherein the volinanserin is administered to post-treat between at least 60 minutes and 240 minutes post to the administration or release of (S)-MDMA. In some embodiments, the serotonin receptor modulator is volinanserin and the psychedelic is (S)-MDMA, wherein the volinanserin is administered to post-treat at least 90 minutes post to the (S)-MDMA. In some embodiments, the serotonin receptor modulator is volinanserin and the psychedelic is (S)-MDMA, wherein the volinanserin is administered to post-treat at least 120 minutes post to the (S)-MDMA.

In some embodiments, the serotonin receptor modulator is volinanserin and the psychedelic is (S)-MDMA, wherein the volinanserin is administered to post-treat at least 150 minutes post to the (S)-MDMA. In some embodiments, the serotonin receptor modulator is volinanserin and the psychedelic is (S)-MDMA, wherein the volinanserin is administered to post-treat between about 15 minutes and about 150 minutes post to the (S)-MDMA. In some embodiments, the serotonin receptor modulator is volinanserin and the psychedelic is (S)-MDMA, wherein the volinanserin is administered to post-treat at least 180 minutes post to the (S)-MDMA. In some embodiments, the serotonin receptor modulator is volinanserin and the psychedelic is (S)-MDMA, wherein the volinanserin is administered to post-treat at least 210 minutes post to the (S)-MDMA. In some embodiments, the serotonin receptor modulator is volinanserin and the psychedelic is (S)-MDMA, wherein the volinanserin is administered to post-treat at least 240 minutes post to the (S)-MDMA. In some embodiments, the serotonin receptor modulator is volinanserin and the psychedelic is (S)-MDMA, wherein the volinanserin is administered to post-treat at least 270 minutes post to the (S)-MDMA. In some embodiments, the serotonin receptor modulator is volinanserin and the psychedelic is (S)-MDMA, wherein the volinanserin is administered to post-treat at least 300 minutes post to the (S)-MDMA. In some embodiments, the serotonin receptor modulator is volinanserin and the psychedelic is (S)-MDMA, wherein the volinanserin is administered to post-treat at least 330 minutes post to the (S)-MDMA. In some embodiments, the serotonin receptor modulator is volinanserin and the psychedelic is (S)-MDMA, wherein the volinanserin is administered to post-treat at least 360 minutes post to the (S)-MDMA. In some preferred embodiments, the serotonin receptor modulator is volinanserin and the psychedelic is (S)-MDMA, wherein volinanserin is administered to post-treat between about 60 minutes and about 180 minutes post to the administration of (S)-MDMA.

In some embodiments, the serotonin receptor modulator is ketanserin and the psychedelic is (S)-MDMA, wherein the ketanserin is administered to post-treat at least 15 minutes post to the administration of (S)-MDMA. In some embodiments, the serotonin receptor modulator is ketanserin and the psychedelic is (S)-MDMA, wherein the ketanserin is administered to post-treat at least 30 minutes post to the (S)-MDMA. In some embodiments, the serotonin receptor modulator is ketanserin and the psychedelic is (S)-MDMA, wherein the ketanserin is administered to post-treat at least 60 minutes post to the (S)-MDMA. In some embodiments, the serotonin receptor modulator is ketanserin and the psychedelic is (S)-MDMA, wherein the ketanserin is administered to post-treat between at least 30 minutes and 360 minutes post to the administration or release (S)-MDMA. In some embodiments, the serotonin receptor modulator is ketanserin and the psychedelic is (S)-MDMA, wherein the ketanserin is administered to post-treat between at least 60 minutes and 240 minutes post to the administration or release of (S)-MDMA. In some embodiments, the serotonin receptor modulator is ketanserin and the psychedelic is (S)-MDMA, wherein the ketanserin is administered to post-treat at least 90 minutes post to the (S)-MDMA. In some embodiments, the serotonin receptor modulator is ketanserin and the psychedelic is (S)-MDMA, wherein the ketanserin is administered to post-treat at least 120 minutes post to the (S)-MDMA. In some embodiments, the serotonin receptor modulator is ketanserin and the psychedelic is (S)-MDMA, wherein the ketanserin is administered to post-treat at least 150 minutes post to the (S)-MDMA. In some embodiments, the serotonin receptor modulator is ketanserin and the psychedelic is (S)-MDMA, wherein the ketanserin is administered to post-treat between about 15 minutes and about 150 minutes post to the (S)-MDMA. In some embodiments, the serotonin receptor modulator is ketanserin and the psychedelic is (S)-MDMA, wherein the ketanserin is administered to post-treat at least 180 minutes post to the (S)-MDMA. In some embodiments, the serotonin receptor modulator is ketanserin and the psychedelic is (S)-MDMA, wherein the ketanserin is administered to post-treat at least 210 minutes post to the (S)-MDMA. In some embodiments, the serotonin receptor modulator is ketanserin and the psychedelic is (S)-MDMA, wherein the ketanserin is administered to post-treat at least 240 minutes post to the (S)-MDMA. In some embodiments, the serotonin receptor modulator is ketanserin and the psychedelic is (S)-MDMA, wherein the ketanserin is administered to post-treat at least 270 minutes post to the (S)-MDMA. In some embodiments, the serotonin receptor modulator is ketanserin and the psychedelic is (S)-MDMA, wherein the ketanserin is administered to post-treat at least 300 minutes post to the (S)-MDMA. In some embodiments, the serotonin receptor modulator is ketanserin and the psychedelic is (S)-MDMA, wherein the ketanserin is administered to post-treat at least 330 minutes post to the (S)-MDMA. In some embodiments, the serotonin receptor modulator is ketanserin and the psychedelic is (S)-MDMA, wherein the ketanserin is administered to post-treat at least 360 minutes post to the (S)-MDMA. In some preferred embodiments, the serotonin receptor modulator is ketanserin and the psychedelic is (S)-MDMA, wherein ketanserin is administered to post-treat between about 60 minutes and about 180 minutes post to the administration of (S)-MDMA.

In some embodiments, the serotonin receptor modulator is ritanserin and the psychedelic is (S)-MDMA, wherein the ritanserin is administered to post-treat at least 15 minutes post to the administration of (S)-MDMA. In some embodiments, the serotonin receptor modulator is ritanserin and the psychedelic is (S)-MDMA, wherein the ritanserin is administered to post-treat at least 30 minutes post to the (S)-MDMA. In some embodiments, the serotonin receptor modulator is ritanserin and the psychedelic is (S)-MDMA, wherein the ritanserin is administered to post-treat at least 60 minutes post to the (S)-MDMA. In some embodiments, the serotonin receptor modulator is ritanserin and the psychedelic is (S)-MDMA, wherein the ritanserin is administered to post-treat between at least 60 minutes and 240 minutes post to the administration or release of (S)-MDMA. In some embodiments, the serotonin receptor modulator is ritanserin and the psychedelic is (S)-MDMA, wherein the ritanserin is administered to post-treat at least 90 minutes post to the (S)-MDMA. In some embodiments, the serotonin receptor modulator is ritanserin and the psychedelic is (S)-MDMA, wherein the ritanserin is administered to post-treat at least 120 minutes post to the (S)-MDMA. In some embodiments, the serotonin receptor modulator is ritanserin and the psychedelic is (S)-MDMA, wherein the ritanserin is administered to post-treat at least 150 minutes post to the (S)-MDMA. In some embodiments, the serotonin receptor modulator is ritanserin and the psychedelic is (S)-MDMA, wherein the ritanserin is administered to post-treat between about 15 minutes and about 150 minutes post to the (S)-MDMA. In some embodiments, the serotonin receptor modulator is ritanserin and the psychedelic is (S)-MDMA, wherein the ritanserin is administered to post-treat at least 180 minutes post to the (S)-MDMA. In some embodiments, the serotonin receptor modulator is ritanserin and the psychedelic is (S)-MDMA, wherein the ritanserin is administered to post-treat at least 210 minutes post to the (S)-MDMA. In some embodiments, the serotonin receptor modulator is ritanserin and the psychedelic is (S)-MDMA, wherein the ritanserin is administered to post-treat at least 240 minutes post to the (S)-MDMA. In some embodiments, the serotonin receptor modulator is ritanserin and the psychedelic is (S)-MDMA, wherein the ritanserin is administered to post-treat at least 270 minutes post to the (S)-MDMA. In some embodiments, the serotonin receptor modulator is ritanserin and the psychedelic is (S)-MDMA, wherein the ritanserin is administered to post-treat at least 300 minutes post to the (S)-MDMA. In some embodiments, the serotonin receptor modulator is ritanserin and the psychedelic is (S)-MDMA, wherein the ritanserin is administered to post-treat at least 330 minutes post to the (S)-MDMA. In some embodiments, the serotonin receptor modulator is ritanserin and the psychedelic is (S)-MDMA, wherein the ritanserin is administered to post-treat at least 360 minutes post to the (S)-MDMA. In some preferred embodiments, the serotonin receptor modulator is ritanserin and the psychedelic is (S)-MDMA, wherein ritanserin is administered to post-treat between about 60 minutes and about 180 minutes post to the administration of (S)-MDMA.

In some embodiments, the serotonin receptor modulator is pimavanserin and the psychedelic is (S)-MDMA, wherein the pimavanserin is administered to post-treat at least 15 minutes post to the administration of (S)-MDMA. In some embodiments, the serotonin receptor modulator is pimavanserin and the psychedelic is (S)-MDMA, wherein the pimavanserin is administered to post-treat at least 30 minutes post to the (S)-MDMA. In some embodiments, the serotonin receptor modulator is pimavanserin and the psychedelic is (S)-MDMA, wherein the pimavanserin is administered to post-treat at least 60 minutes post to the (S)-MDMA. In some embodiments, the serotonin receptor modulator is pimavanserin and the psychedelic is (S)-MDMA, wherein the pimavanserin is administered to post-treat between at least 60 minutes and 240 minutes post to the administration or release of (S)-MDMA. In some embodiments, the serotonin receptor modulator is pimavanserin and the psychedelic is (S)-MDMA, wherein the pimavanserin is administered to post-treat at least 90 minutes post to the (S)-MDMA. In some embodiments, the serotonin receptor modulator is pimavanserin and the psychedelic is (S)-MDMA, wherein the pimavanserin is administered to post-treat at least 120 minutes post to the (S)-MDMA. In some embodiments, the serotonin receptor modulator is pimavanserin and the psychedelic is (S)-MDMA, wherein the pimavanserin is administered to post-treat at least 150 minutes post to the (S)-MDMA. In some embodiments, the serotonin receptor modulator is pimavanserin and the psychedelic is (S)-MDMA, wherein the pimavanserin is administered to post-treat between about 15 minutes and about 150 minutes post to the (S)-MDMA. In some embodiments, the serotonin receptor modulator is pimavanserin and the psychedelic is (S)-MDMA, wherein the pimavanserin is administered to post-treat at least 180 minutes post to the (S)-MDMA. In some embodiments, the serotonin receptor modulator is pimavanserin and the psychedelic is (S)-MDMA, wherein the pimavanserin is administered to post-treat at least 210 minutes post to the (S)-MDMA. In some embodiments, the serotonin receptor modulator is pimavanserin and the psychedelic is (S)-MDMA, wherein the pimavanserin is administered to post-treat at least 240 minutes post to the (S)-MDMA. In some embodiments, the serotonin receptor modulator is pimavanserin and the psychedelic is (S)-MDMA, wherein the pimavanserin is administered to post-treat at least 270 minutes post to the (S)-MDMA. In some embodiments, the serotonin receptor modulator is pimavanserin and the psychedelic is (S)-MDMA, wherein the pimavanserin is administered to post-treat at least 300 minutes post to the (S)-MDMA. In some embodiments, the serotonin receptor modulator is pimavanserin and the psychedelic is (S)-MDMA, wherein the pimavanserin is administered to post-treat at least 330 minutes post to the (S)-MDMA. In some embodiments, the serotonin receptor modulator is pimavanserin and the psychedelic is (S)-MDMA, wherein the pimavanserin is administered to post-treat at least 360 minutes post to the (S)-MDMA. In some preferred embodiments, the serotonin receptor modulator is pimavanserin and the psychedelic is (S)-MDMA, wherein pimavanserin is administered to post-treat between about 60 minutes and about 180 minutes post to the administration of (S)-MDMA.

In some embodiments, the serotonin receptor modulator is nelotanserin and the psychedelic is (S)-MDMA, wherein the nelotanserin is administered to post-treat at least 15 minutes post to the administration of (S)-MDMA. In some embodiments, the serotonin receptor modulator is nelotanserin and the psychedelic is (S)-MDMA, wherein the nelotanserin is administered to post-treat at least 30 minutes post to the (S)-MDMA. In some embodiments, the serotonin receptor modulator is nelotanserin and the psychedelic is (S)-MDMA, wherein the nelotanserin is administered to post-treat at least 60 minutes post to the (S)-MDMA. In some embodiments, the serotonin receptor modulator is nelotanserin and the psychedelic is (S)-MDMA, wherein the nelotanserin is administered to post-treat between at least 60 minutes and 240 minutes post to the administration or release of (S)-MDMA. In some embodiments, the serotonin receptor modulator is nelotanserin and the psychedelic is (S)-MDMA, wherein the nelotanserin is administered to post-treat at least 90 minutes post to the (S)-MDMA. In some embodiments, the serotonin receptor modulator is nelotanserin and the psychedelic is (S)-MDMA, wherein the nelotanserin is administered to post-treat at least 120 minutes post to the (S)-MDMA.

In some embodiments, the serotonin receptor modulator is nelotanserin and the psychedelic is (S)-MDMA, wherein the nelotanserin is administered to post-treat at least 150 minutes post to the (S)-MDMA. In some embodiments, the serotonin receptor modulator is nelotanserin and the psychedelic is (S)-MDMA, wherein the nelotanserin is administered to post-treat between about 15 minutes and about 150 minutes post to the (S)-MDMA. In some embodiments, the serotonin receptor modulator is nelotanserin and the psychedelic is (S)-MDMA, wherein the nelotanserin is administered to post-treat at least 180 minutes post to the (S)-MDMA. In some embodiments, the serotonin receptor modulator is nelotanserin and the psychedelic is (S)-MDMA, wherein the nelotanserin is administered to post-treat at least 210 minutes post to the (S)-MDMA. In some embodiments, the serotonin receptor modulator is nelotanserin and the psychedelic is (S)-MDMA, wherein the nelotanserin is administered to post-treat at least 240 minutes post to the (S)-MDMA. In some embodiments, the serotonin receptor modulator is nelotanserin and the psychedelic is (S)-MDMA, wherein the nelotanserin is administered to post-treat at least 270 minutes post to the (S)-MDMA.

In some embodiments, the serotonin receptor modulator is nelotanserin and the psychedelic is (S)-MDMA, wherein the nelotanserin is administered to post-treat at least 300 minutes post to the (S)-MDMA. In some embodiments, the serotonin receptor modulator is nelotanserin and the psychedelic is (S)-MDMA, wherein the nelotanserin is administered to post-treat at least 330 minutes post to the (S)-MDMA. In some embodiments, the serotonin receptor modulator is nelotanserin and the psychedelic is (S)-MDMA, wherein the nelotanserin is administered to post-treat at least 360 minutes post to the (S)-MDMA. In some preferred embodiments, the serotonin receptor modulator is nelotanserin and the psychedelic is (S)-MDMA, wherein nelotanserin is administered to post-treat between about 60 minutes and about 180 minutes post to the administration of (S)-MDMA.

In some embodiments, the serotonin receptor modulator is pruvanserin and the psychedelic is (S)-MDMA, wherein the pruvanserin is administered to post-treat at least 15 minutes post to the administration of (S)-MDMA. In some embodiments, the serotonin receptor modulator is pruvanserin and the psychedelic is (S)-MDMA, wherein the pruvanserin is administered to post-treat at least 30 minutes post to the (S)-MDMA. In some embodiments, the serotonin receptor modulator is pruvanserin and the psychedelic is (S)-MDMA, wherein the pruvanserin is administered to post-treat at least 60 minutes post to the (S)-MDMA. In some embodiments, the serotonin receptor modulator is pruvanserin and the psychedelic is (S)-MDMA, wherein the pruvanserin is administered to post-treat between at least 60 minutes and 240 minutes post to the administration or release of (S)-MDMA. In some embodiments, the serotonin receptor modulator is pruvanserin and the psychedelic is (S)-MDMA, wherein the pruvanserin is administered to post-treat at least 90 minutes post to the (S)-MDMA. In some embodiments, the serotonin receptor modulator is pruvanserin and the psychedelic is (S)-MDMA, wherein the pruvanserin is administered to post-treat at least 120 minutes post to the (S)-MDMA. In some embodiments, the serotonin receptor modulator is pruvanserin and the psychedelic is (S)-MDMA, wherein the pruvanserin is administered to post-treat at least 150 minutes post to the (S)-MDMA. In some embodiments, the serotonin receptor modulator is pruvanserin and the psychedelic is (S)-MDMA, wherein the pruvanserin is administered to post-treat between about 15 minutes and about 150 minutes post to the (S)-MDMA. In some embodiments, the serotonin receptor modulator is pruvanserin and the psychedelic is (S)-MDMA, wherein the pruvanserin is administered to post-treat at least 180 minutes post to the (S)-MDMA. In some embodiments, the serotonin receptor modulator is pruvanserin and the psychedelic is (S)-MDMA, wherein the pruvanserin is administered to post-treat at least 210 minutes post to the (S)-MDMA.

In some embodiments, the serotonin receptor modulator is pruvanserin and the psychedelic is (S)-MDMA, wherein the pruvanserin is administered to post-treat at least 240 minutes post to the (S)-MDMA. In some embodiments, the serotonin receptor modulator is pruvanserin and the psychedelic is (S)-MDMA, wherein the pruvanserin is administered to post-treat at least 270 minutes post to the (S)-MDMA. In some embodiments, the serotonin receptor modulator is pruvanserin and the psychedelic is (S)-MDMA, wherein the pruvanserin is administered to post-treat at least 300 minutes post to the (S)-MDMA. In some embodiments, the serotonin receptor modulator is pruvanserin and the psychedelic is (S)-MDMA, wherein the pruvanserin is administered to post-treat at least 330 minutes post to the (S)-MDMA. In some embodiments, the serotonin receptor modulator is pruvanserin and the psychedelic is (S)-MDMA, wherein the pruvanserin is administered to post-treat at least 360 minutes post to the (S)-MDMA. In some preferred embodiments, the serotonin receptor modulator is pruvanserin and the psychedelic is (S)-MDMA, wherein pruvanserin is administered to post-treat between about 60 minutes and about 180 minutes post to the administration of (S)-MDMA.

In some embodiments, the serotonin receptor modulator is flibanserin and the psychedelic is (S)-MDMA, wherein the flibanserin is administered to post-treat at least 15 minutes post to the administration of (S)-MDMA. In some embodiments, the serotonin receptor modulator is flibanserin and the psychedelic is (S)-MDMA, wherein the flibanserin is administered to post-treat at least 30 minutes post to the (S)-MDMA. In some embodiments, the serotonin receptor modulator is flibanserin and the psychedelic is (S)-MDMA, wherein the flibanserin is administered to post-treat at least 60 minutes post to the (S)-MDMA. In some embodiments, the serotonin receptor modulator is flibanserin and the psychedelic is (S)-MDMA, wherein the flibanserin is administered to post-treat at least 90 minutes post to the (S)-MDMA. In some embodiments, the serotonin receptor modulator is flibanserin and the psychedelic is (S)-MDMA, wherein the flibanserin is administered to post-treat at least 120 minutes post to the (S)-MDMA. In some embodiments, the serotonin receptor modulator is flibanserin and the psychedelic is (S)-MDMA, wherein the flibanserin is administered to post-treat at least 150 minutes post to the (S)-MDMA. In some embodiments, the serotonin receptor modulator is flibanserin and the psychedelic is (S)-MDMA, wherein the flibanserin is administered to post-treat between about 15 minutes and about 150 minutes post to the (S)-MDMA. In some embodiments, the serotonin receptor modulator is flibanserin and the psychedelic is (S)-MDMA, wherein the flibanserin is administered to post-treat at least 180 minutes post to the (S)-MDMA. In some embodiments, the serotonin receptor modulator is flibanserin and the psychedelic is (S)-MDMA, wherein the flibanserin is administered to post-treat at least 210 minutes post to the (S)-MDMA. In some embodiments, the serotonin receptor modulator is flibanserin and the psychedelic is (S)-MDMA, wherein the flibanserin is administered to post-treat at least 240 minutes post to the (S)-MDMA. In some embodiments, the serotonin receptor modulator is flibanserin and the psychedelic is (S)-MDMA, wherein the flibanserin is administered to post-treat at least 270 minutes post to the (S)-MDMA. In some embodiments, the serotonin receptor modulator is flibanserin and the psychedelic is (S)-MDMA, wherein the flibanserin is administered to post-treat at least 300 minutes post to the (S)-MDMA. In some embodiments, the serotonin receptor modulator is flibanserin and the psychedelic is (S)-MDMA, wherein the flibanserin is administered to post-treat at least 330 minutes post to the (S)-MDMA. In some embodiments, the serotonin receptor modulator is flibanserin and the psychedelic is (S)-MDMA, wherein the flibanserin is administered to post-treat at least 360 minutes post to the (S)-MDMA. In some preferred embodiments, the serotonin receptor modulator is flibanserin and the psychedelic is (S)-MDMA, wherein flibanserin is administered to post-treat between about 60 minutes and about 180 minutes post to the administration of (S)-MDMA.

In some embodiments, the serotonin receptor modulator is olanzapine and the psychedelic is (S)-MDMA, wherein the olanzapine is administered to post-treat at least 15 minutes post to the administration of (S)-MDMA. In some embodiments, the serotonin receptor modulator is olanzapine and the psychedelic is (S)-MDMA, wherein the olanzapine is administered to post-treat at least 30 minutes post to the (S)-MDMA. In some embodiments, the serotonin receptor modulator is olanzapine and the psychedelic is (S)-MDMA, wherein the olanzapine is administered to post-treat at least 60 minutes post to the (S)-MDMA. In some embodiments, the serotonin receptor modulator is olanzapine and the psychedelic is (S)-MDMA, wherein the olanzapine is administered to post-treat at least 90 minutes post to the (S)-MDMA. In some embodiments, the serotonin receptor modulator is olanzapine and the psychedelic is (S)-MDMA, wherein the olanzapine is administered to post-treat at least 120 minutes post to the (S)-MDMA. In some embodiments, the serotonin receptor modulator is olanzapine and the psychedelic is (S)-MDMA, wherein the olanzapine is administered to post-treat at least 150 minutes post to the (S)-MDMA. In some embodiments, the serotonin receptor modulator is olanzapine and the psychedelic is (S)-MDMA, wherein the olanzapine is administered to post-treat between about 15 minutes and about 150 minutes post to the (S)-MDMA. In some embodiments, the serotonin receptor modulator is olanzapine and the psychedelic is (S)-MDMA, wherein the olanzapine is administered to post-treat at least 180 minutes post to the (S)-MDMA. In some embodiments, the serotonin receptor modulator is olanzapine and the psychedelic is (S)-MDMA, wherein the olanzapine is administered to post-treat at least 210 minutes post to the (S)-MDMA. In some embodiments, the serotonin receptor modulator is olanzapine and the psychedelic is (S)-MDMA, wherein the olanzapine is administered to post-treat at least 240 minutes post to the (S)-MDMA. In some embodiments, the serotonin receptor modulator is olanzapine and the psychedelic is (S)-MDMA, wherein the olanzapine is administered to post-treat at least 270 minutes post to the (S)-MDMA. In some embodiments, the serotonin receptor modulator is olanzapine and the psychedelic is (S)-MDMA, wherein the olanzapine is administered to post-treat at least 300 minutes post to the (S)-MDMA. In some embodiments, the serotonin receptor modulator is olanzapine and the psychedelic is (S)-MDMA, wherein the olanzapine is administered to post-treat at least 330 minutes post to the (S)-MDMA. In some embodiments, the serotonin receptor modulator is olanzapine and the psychedelic is (S)-MDMA, wherein the olanzapine is administered to post-treat at least 360 minutes post to the (S)-MDMA. In some preferred embodiments, the serotonin receptor modulator is olanzapine and the psychedelic is (S)-MDMA, wherein olanzapine is administered to post-treat between about 60 minutes and about 180 minutes post to the administration of (S)-MDMA.

In some embodiments, the serotonin receptor modulator is quetiapine and the psychedelic is (S)-MDMA, wherein the quetiapine is administered to post-treat at least 15 minutes post to the administration of (S)-MDMA. In some embodiments, the serotonin receptor modulator is quetiapine and the psychedelic is (S)-MDMA, wherein the quetiapine is administered to post-treat at least 30 minutes post to the (S)-MDMA. In some embodiments, the serotonin receptor modulator is quetiapine and the psychedelic is (S)-MDMA, wherein the quetiapine is administered to post-treat at least 60 minutes post to the (S)-MDMA. In some embodiments, the serotonin receptor modulator is quetiapine and the psychedelic is (S)-MDMA, wherein the quetiapine is administered to post-treat at least 90 minutes post to the (S)-MDMA. In some embodiments, the serotonin receptor modulator is quetiapine and the psychedelic is (S)-MDMA, wherein the quetiapine is administered to post-treat at least 120 minutes post to the (S)-MDMA. In some embodiments, the serotonin receptor modulator is quetiapine and the psychedelic is (S)-MDMA, wherein the quetiapine is administered to post-treat at least 150 minutes post to the (S)-MDMA. In some embodiments, the serotonin receptor modulator is quetiapine and the psychedelic is (S)-MDMA, wherein the quetiapine is administered to post-treat between about 15 minutes and about 150 minutes post to the (S)-MDMA. In some embodiments, the serotonin receptor modulator is quetiapine and the psychedelic is (S)-MDMA, wherein the quetiapine is administered to post-treat at least 180 minutes post to the (S)-MDMA. In some embodiments, the serotonin receptor modulator is quetiapine and the psychedelic is (S)-MDMA, wherein the quetiapine is administered to post-treat at least 210 minutes post to the (S)-MDMA. In some embodiments, the serotonin receptor modulator is quetiapine and the psychedelic is (S)-MDMA, wherein the quetiapine is administered to post-treat at least 240 minutes post to the (S)-MDMA. In some embodiments, the serotonin receptor modulator is quetiapine and the psychedelic is (S)-MDMA, wherein the quetiapine is administered to post-treat at least 270 minutes post to the (S)-MDMA. In some embodiments, the serotonin receptor modulator is quetiapine and the psychedelic is (S)-MDMA, wherein the quetiapine is administered to post-treat at least 300 minutes post to the (S)-MDMA. In some embodiments, the serotonin receptor modulator is quetiapine and the psychedelic is (S)-MDMA, wherein the quetiapine is administered to post-treat at least 330 minutes post to the (S)-MDMA. In some embodiments, the serotonin receptor modulator is quetiapine and the psychedelic is (S)-MDMA, wherein the quetiapine is administered to post-treat at least 360 minutes post to the (S)-MDMA. In some preferred embodiments, the serotonin receptor modulator is quetiapine and the psychedelic is (S)-MDMA, wherein quetiapine is administered to post-treat between about 60 minutes and about 180 minutes post to the administration of (S)-MDMA.

In some embodiments, the serotonin receptor modulator is risperidone and the psychedelic is (S)-MDMA, wherein the risperidone is administered to post-treat at least 15 minutes post to the administration of (S)-MDMA. In some embodiments, the serotonin receptor modulator is risperidone and the psychedelic is (S)-MDMA, wherein the risperidone is administered to post-treat at least 30 minutes post to the (S)-MDMA. In some embodiments, the serotonin receptor modulator is risperidone and the psychedelic is (S)-MDMA, wherein the risperidone is administered to post-treat at least 60 minutes post to the (S)-MDMA. In some embodiments, the serotonin receptor modulator is risperidone and the psychedelic is (S)-MDMA, wherein the risperidone is administered to post-treat at least 90 minutes post to the (S)-MDMA. In some embodiments, the serotonin receptor modulator is risperidone and the psychedelic is (S)-MDMA, wherein the risperidone is administered to post-treat at least 120 minutes post to the (S)-MDMA. In some embodiments, the serotonin receptor modulator is risperidone and the psychedelic is (S)-MDMA, wherein the risperidone is administered to post-treat at least 150 minutes post to the (S)-MDMA. In some embodiments, the serotonin receptor modulator is risperidone and the psychedelic is (S)-MDMA, wherein the risperidone is administered to post-treat between about 15 minutes and about 150 minutes post to the (S)-MDMA. In some embodiments, the serotonin receptor modulator is risperidone and the psychedelic is (S)-MDMA, wherein the risperidone is administered to post-treat at least 180 minutes post to the (S)-MDMA. In some embodiments, the serotonin receptor modulator is risperidone and the psychedelic is (S)-MDMA, wherein the risperidone is administered to post-treat at least 210 minutes post to the (S)-MDMA. In some embodiments, the serotonin receptor modulator is risperidone and the psychedelic is (S)-MDMA, wherein the risperidone is administered to post-treat at least 240 minutes post to the (S)-MDMA. In some embodiments, the serotonin receptor modulator is risperidone and the psychedelic is (S)-MDMA, wherein the risperidone is administered to post-treat at least 270 minutes post to the (S)-MDMA. In some embodiments, the serotonin receptor modulator is risperidone and the psychedelic is (S)-MDMA, wherein the risperidone is administered to post-treat at least 300 minutes post to the (S)-MDMA. In some embodiments, the serotonin receptor modulator is risperidone and the psychedelic is (S)-MDMA, wherein the risperidone is administered to post-treat at least 330 minutes post to the (S)-MDMA. In some embodiments, the serotonin receptor modulator is risperidone and the psychedelic is (S)-MDMA, wherein the risperidone is administered to post-treat at least 360 minutes post to the (S)-MDMA. In some preferred embodiments, the serotonin receptor modulator is risperidone and the psychedelic is (S)-MDMA, wherein risperidone is administered to post-treat between about 60 minutes and about 180 minutes post to the administration of (S)-MDMA.

In some embodiments, the serotonin receptor modulator is eplivanserin and the psychedelic is MBDB, wherein the eplivanserin is administered to post-treat at least 15 minutes post to the administration of MBDB. In some embodiments, the serotonin receptor modulator is eplivanserin and the psychedelic is MBDB, wherein the eplivanserin is administered to post-treat at least 30 minutes post to the MBDB. In some embodiments, the serotonin receptor modulator is eplivanserin and the psychedelic is MBDB, wherein the eplivanserin is administered to post-treat at least 60 minutes post to the MBDB. In some embodiments, the serotonin receptor modulator is eplivanserin and the psychedelic is MBDB, wherein the eplivanserin is administered to post-treat at least 90 minutes post to the MBDB. In some embodiments, the serotonin receptor modulator is eplivanserin and the psychedelic is MBDB, wherein the eplivanserin is administered to post-treat between at least 30 minutes post and 360 minutes post to the release or administration of the MBDB. In some embodiments, the serotonin receptor modulator is eplivanserin and the psychedelic is MBDB, wherein the eplivanserin is administered to post-treat between at least 60 minutes post and 360 minutes post to the release or administration the MBDB. In some embodiments, the serotonin receptor modulator is eplivanserin and the psychedelic is MBDB, wherein the eplivanserin is administered to post-treat between at least 90 minutes and 240 minutes post to the MBDB. In some embodiments, the serotonin receptor modulator is eplivanserin and the psychedelic is MBDB, wherein the eplivanserin is administered to post-treat at least 120 minutes post to the MBDB. In some embodiments, the serotonin receptor modulator is eplivanserin and the psychedelic is MBDB, wherein the eplivanserin is administered to post-treat at least 150 minutes post to the MBDB. In some embodiments, the serotonin receptor modulator is eplivanserin and the psychedelic is MBDB, wherein the eplivanserin is administered to post-treat between about 15 minutes and about 150 minutes post to the MBDB. In some embodiments, the serotonin receptor modulator is eplivanserin and the psychedelic is MBDB, wherein the eplivanserin is administered to post-treat at least 180 minutes post to the MBDB. In some embodiments, the serotonin receptor modulator is eplivanserin and the psychedelic is MBDB, wherein the eplivanserin is administered to post-treat at least 210 minutes post to the MBDB. In some embodiments, the serotonin receptor modulator is eplivanserin and the psychedelic is MBDB, wherein the eplivanserin is administered to post-treat at least 240 minutes post to the MBDB.

In some embodiments, the serotonin receptor modulator is eplivanserin and the psychedelic is MBDB, wherein the eplivanserin is administered to post-treat at least 270 minutes post to the MBDB. In some embodiments, the serotonin receptor modulator is eplivanserin and the psychedelic is MBDB, wherein the eplivanserin is administered to post-treat at least 300 minutes post to the MBDB. In some embodiments, the serotonin receptor modulator is eplivanserin and the psychedelic is MBDB, wherein the eplivanserin is administered to post-treat at least 330 minutes post to the MBDB. In some embodiments, the serotonin receptor modulator is eplivanserin and the psychedelic is MBDB, wherein the eplivanserin is administered to post-treat at least 360 minutes post to the MBDB.

In some preferred embodiments, the serotonin receptor modulator is eplivanserin and the psychedelic is MBDB, wherein eplivanserin is administered to post-treat between about 60 minutes and about 180 minutes post to the administration of MBDB.

In some embodiments, the serotonin receptor modulator is volinanserin and the psychedelic is MBDB, wherein the volinanserin is administered to post-treat at least 15 minutes post to the administration of MBDB. In some embodiments, the serotonin receptor modulator is volinanserin and the psychedelic is MBDB, wherein the volinanserin is administered to post-treat at least 30 minutes post to the MBDB. In some embodiments, the serotonin receptor modulator is volinanserin and the psychedelic is MBDB, wherein the volinanserin is administered to post-treat at least 60 minutes post to the MBDB. In some embodiments, the serotonin receptor modulator is volinanserin and the psychedelic is MBDB, wherein the volinanserin is administered to post-treat a subject between at least 15 minutes and 360 minutes post to the administration or release of MBDB. In some embodiments, the serotonin receptor modulator is volinanserin and the psychedelic is MBDB, wherein the volinanserin is administered to post-treat between at least 30 minutes and 360 minutes post to the administration or release of MBDB. In some embodiments, the serotonin receptor modulator is volinanserin and the psychedelic is MBDB, wherein the volinanserin is administered to post-treat between at least 60 minutes and 240 minutes post to the administration or release of MBDB. In some embodiments, the serotonin receptor modulator is volinanserin and the psychedelic is MBDB, wherein the volinanserin is administered to post-treat at least 90 minutes post to the MBDB. In some embodiments, the serotonin receptor modulator is volinanserin and the psychedelic is MBDB, wherein the volinanserin is administered to post-treat at least 120 minutes post to the MBDB.

In some embodiments, the serotonin receptor modulator is volinanserin and the psychedelic is MBDB, wherein the volinanserin is administered to post-treat at least 150 minutes post to the MBDB. In some embodiments, the serotonin receptor modulator is volinanserin and the psychedelic is MBDB, wherein the volinanserin is administered to post-treat between about 15 minutes and about 150 minutes post to the MBDB. In some embodiments, the serotonin receptor modulator is volinanserin and the psychedelic is MBDB, wherein the volinanserin is administered to post-treat at least 180 minutes post to the MBDB. In some embodiments, the serotonin receptor modulator is volinanserin and the psychedelic is MBDB, wherein the volinanserin is administered to post-treat at least 210 minutes post to the MBDB. In some embodiments, the serotonin receptor modulator is volinanserin and the psychedelic is MBDB, wherein the volinanserin is administered to post-treat at least 240 minutes post to the MBDB. In some embodiments, the serotonin receptor modulator is volinanserin and the psychedelic is MBDB, wherein the volinanserin is administered to post-treat at least 270 minutes post to the MBDB. In some embodiments, the serotonin receptor modulator is volinanserin and the psychedelic is MBDB, wherein the volinanserin is administered to post-treat at least 300 minutes post to the MBDB. In some embodiments, the serotonin receptor modulator is volinanserin and the psychedelic is MBDB, wherein the volinanserin is administered to post-treat at least 330 minutes post to the MBDB. In some embodiments, the serotonin receptor modulator is volinanserin and the psychedelic is MBDB, wherein the volinanserin is administered to post-treat at least 360 minutes post to the MBDB. In some preferred embodiments, the serotonin receptor modulator is volinanserin and the psychedelic is MBDB, wherein volinanserin is administered to post-treat between about 60 minutes and about 180 minutes post to the administration of MBDB.

In some embodiments, the serotonin receptor modulator is ketanserin and the psychedelic is MBDB, wherein the ketanserin is administered to post-treat at least 15 minutes post to the administration of MBDB. In some embodiments, the serotonin receptor modulator is ketanserin and the psychedelic is MBDB, wherein the ketanserin is administered to post-treat at least 30 minutes post to the MBDB. In some embodiments, the serotonin receptor modulator is ketanserin and the psychedelic is MBDB, wherein the ketanserin is administered to post-treat at least 60 minutes post to the MBDB. In some embodiments, the serotonin receptor modulator is ketanserin and the psychedelic is MBDB, wherein the ketanserin is administered to post-treat between at least 30 minutes and 360 minutes post to the administration or release MBDB. In some embodiments, the serotonin receptor modulator is ketanserin and the psychedelic is MBDB, wherein the ketanserin is administered to post-treat between at least 60 minutes and 240 minutes post to the administration or release of MBDB. In some embodiments, the serotonin receptor modulator is ketanserin and the psychedelic is MBDB, wherein the ketanserin is administered to post-treat at least 90 minutes post to the MBDB. In some embodiments, the serotonin receptor modulator is ketanserin and the psychedelic is MBDB, wherein the ketanserin is administered to post-treat at least 120 minutes post to the MBDB. In some embodiments, the serotonin receptor modulator is ketanserin and the psychedelic is MBDB, wherein the ketanserin is administered to post-treat at least 150 minutes post to the MBDB. In some embodiments, the serotonin receptor modulator is ketanserin and the psychedelic is MBDB, wherein the ketanserin is administered to post-treat between about 15 minutes and about 150 minutes post to the MBDB. In some embodiments, the serotonin receptor modulator is ketanserin and the psychedelic is MBDB, wherein the ketanserin is administered to post-treat at least 180 minutes post to the MBDB. In some embodiments, the serotonin receptor modulator is ketanserin and the psychedelic is MBDB, wherein the ketanserin is administered to post-treat at least 210 minutes post to the MBDB. In some embodiments, the serotonin receptor modulator is ketanserin and the psychedelic is MBDB, wherein the ketanserin is administered to post-treat at least 240 minutes post to the MBDB. In some embodiments, the serotonin receptor modulator is ketanserin and the psychedelic is MBDB, wherein the ketanserin is administered to post-treat at least 270 minutes post to the MBDB. In some embodiments, the serotonin receptor modulator is ketanserin and the psychedelic is MBDB, wherein the ketanserin is administered to post-treat at least 300 minutes post to the MBDB. In some embodiments, the serotonin receptor modulator is ketanserin and the psychedelic is MBDB, wherein the ketanserin is administered to post-treat at least 330 minutes post to the MBDB. In some embodiments, the serotonin receptor modulator is ketanserin and the psychedelic is MBDB, wherein the ketanserin is administered to post-treat at least 360 minutes post to the MBDB. In some preferred embodiments, the serotonin receptor modulator is ketanserin and the psychedelic is MBDB, wherein ketanserin is administered to post-treat between about 60 minutes and about 180 minutes post to the administration of MBDB.

In some embodiments, the serotonin receptor modulator is ritanserin and the psychedelic is MBDB, wherein the ritanserin is administered to post-treat at least 15 minutes post to the administration of MBDB. In some embodiments, the serotonin receptor modulator is ritanserin and the psychedelic is MBDB, wherein the ritanserin is administered to post-treat at least 30 minutes post to the MBDB. In some embodiments, the serotonin receptor modulator is ritanserin and the psychedelic is MBDB, wherein the ritanserin is administered to post-treat at least 60 minutes post to the MBDB. In some embodiments, the serotonin receptor modulator is ritanserin and the psychedelic is MBDB, wherein the ritanserin is administered to post-treat between at least 60 minutes and 240 minutes post to the administration or release of MBDB. In some embodiments, the serotonin receptor modulator is ritanserin and the psychedelic is MBDB, wherein the ritanserin is administered to post-treat at least 90 minutes post to the MBDB. In some embodiments, the serotonin receptor modulator is ritanserin and the psychedelic is MBDB, wherein the ritanserin is administered to post-treat at least 120 minutes post to the MBDB. In some embodiments, the serotonin receptor modulator is ritanserin and the psychedelic is MBDB, wherein the ritanserin is administered to post-treat at least 150 minutes post to the MBDB. In some embodiments, the serotonin receptor modulator is ritanserin and the psychedelic is MBDB, wherein the ritanserin is administered to post-treat between about 15 minutes and about 150 minutes post to the MBDB. In some embodiments, the serotonin receptor modulator is ritanserin and the psychedelic is MBDB, wherein the ritanserin is administered to post-treat at least 180 minutes post to the MBDB. In some embodiments, the serotonin receptor modulator is ritanserin and the psychedelic is MBDB, wherein the ritanserin is administered to post-treat at least 210 minutes post to the MBDB. In some embodiments, the serotonin receptor modulator is ritanserin and the psychedelic is MBDB, wherein the ritanserin is administered to post-treat at least 240 minutes post to the MBDB. In some embodiments, the serotonin receptor modulator is ritanserin and the psychedelic is MBDB, wherein the ritanserin is administered to post-treat at least 270 minutes post to the MBDB. In some embodiments, the serotonin receptor modulator is ritanserin and the psychedelic is MBDB, wherein the ritanserin is administered to post-treat at least 300 minutes post to the MBDB. In some embodiments, the serotonin receptor modulator is ritanserin and the psychedelic is MBDB, wherein the ritanserin is administered to post-treat at least 330 minutes post to the MBDB. In some embodiments, the serotonin receptor modulator is ritanserin and the psychedelic is MBDB, wherein the ritanserin is administered to post-treat at least 360 minutes post to the MBDB. In some preferred embodiments, the serotonin receptor modulator is ritanserin and the psychedelic is MBDB, wherein ritanserin is administered to post-treat between about 60 minutes and about 180 minutes post to the administration of MBDB.

In some embodiments, the serotonin receptor modulator is pimavanserin and the psychedelic is MBDB, wherein the pimavanserin is administered to post-treat at least 15 minutes post to the administration of MBDB. In some embodiments, the serotonin receptor modulator is pimavanserin and the psychedelic is MBDB, wherein the pimavanserin is administered to post-treat at least 30 minutes post to the MBDB. In some embodiments, the serotonin receptor modulator is pimavanserin and the psychedelic is MBDB, wherein the pimavanserin is administered to post-treat at least 60 minutes post to the MBDB. In some embodiments, the serotonin receptor modulator is pimavanserin and the psychedelic is MBDB, wherein the pimavanserin is administered to post-treat between at least 60 minutes and 240 minutes post to the administration or release of MBDB. In some embodiments, the serotonin receptor modulator is pimavanserin and the psychedelic is MBDB, wherein the pimavanserin is administered to post-treat at least 90 minutes post to the MBDB. In some embodiments, the serotonin receptor modulator is pimavanserin and the psychedelic is MBDB, wherein the pimavanserin is administered to post-treat at least 120 minutes post to the MBDB. In some embodiments, the serotonin receptor modulator is pimavanserin and the psychedelic is MBDB, wherein the pimavanserin is administered to post-treat at least 150 minutes post to the MBDB. In some embodiments, the serotonin receptor modulator is pimavanserin and the psychedelic is MBDB, wherein the pimavanserin is administered to post-treat between about 15 minutes and about 150 minutes post to the MBDB. In some embodiments, the serotonin receptor modulator is pimavanserin and the psychedelic is MBDB, wherein the pimavanserin is administered to post-treat at least 180 minutes post to the MBDB. In some embodiments, the serotonin receptor modulator is pimavanserin and the psychedelic is MBDB, wherein the pimavanserin is administered to post-treat at least 210 minutes post to the MBDB. In some embodiments, the serotonin receptor modulator is pimavanserin and the psychedelic is MBDB, wherein the pimavanserin is administered to post-treat at least 240 minutes post to the MBDB. In some embodiments, the serotonin receptor modulator is pimavanserin and the psychedelic is MBDB, wherein the pimavanserin is administered to post-treat at least 270 minutes post to the MBDB. In some embodiments, the serotonin receptor modulator is pimavanserin and the psychedelic is MBDB, wherein the pimavanserin is administered to post-treat at least 300 minutes post to the MBDB. In some embodiments, the serotonin receptor modulator is pimavanserin and the psychedelic is MBDB, wherein the pimavanserin is administered to post-treat at least 330 minutes post to the MBDB. In some embodiments, the serotonin receptor modulator is pimavanserin and the psychedelic is MBDB, wherein the pimavanserin is administered to post-treat at least 360 minutes post to the MBDB. In some preferred embodiments, the serotonin receptor modulator is pimavanserin and the psychedelic is MBDB, wherein pimavanserin is administered to post-treat between about 60 minutes and about 180 minutes post to the administration of MBDB.

In some embodiments, the serotonin receptor modulator is nelotanserin and the psychedelic is MBDB, wherein the nelotanserin is administered to post-treat at least 15 minutes post to the administration of MBDB. In some embodiments, the serotonin receptor modulator is nelotanserin and the psychedelic is MBDB, wherein the nelotanserin is administered to post-treat at least 30 minutes post to the MBDB. In some embodiments, the serotonin receptor modulator is nelotanserin and the psychedelic is MBDB, wherein the nelotanserin is administered to post-treat at least 60 minutes post to the MBDB. In some embodiments, the serotonin receptor modulator is nelotanserin and the psychedelic is MBDB, wherein the nelotanserin is administered to post-treat between at least 60 minutes and 240 minutes post to the administration or release of MBDB. In some embodiments, the serotonin receptor modulator is nelotanserin and the psychedelic is MBDB, wherein the nelotanserin is administered to post-treat at least 90 minutes post to the MBDB. In some embodiments, the serotonin receptor modulator is nelotanserin and the psychedelic is MBDB, wherein the nelotanserin is administered to post-treat at least 120 minutes post to the MBDB.

In some embodiments, the serotonin receptor modulator is nelotanserin and the psychedelic is MBDB, wherein the nelotanserin is administered to post-treat at least 150 minutes post to the MBDB. In some embodiments, the serotonin receptor modulator is nelotanserin and the psychedelic is MBDB, wherein the nelotanserin is administered to post-treat between about 15 minutes and about 150 minutes post to the MBDB. In some embodiments, the serotonin receptor modulator is nelotanserin and the psychedelic is MBDB, wherein the nelotanserin is administered to post-treat at least 180 minutes post to the MBDB. In some embodiments, the serotonin receptor modulator is nelotanserin and the psychedelic is MBDB, wherein the nelotanserin is administered to post-treat at least 210 minutes post to the MBDB. In some embodiments, the serotonin receptor modulator is nelotanserin and the psychedelic is MBDB, wherein the nelotanserin is administered to post-treat at least 240 minutes post to the MBDB. In some embodiments, the serotonin receptor modulator is nelotanserin and the psychedelic is MBDB, wherein the nelotanserin is administered to post-treat at least 270 minutes post to the MBDB.

In some embodiments, the serotonin receptor modulator is nelotanserin and the psychedelic is MBDB, wherein the nelotanserin is administered to post-treat at least 300 minutes post to the MBDB. In some embodiments, the serotonin receptor modulator is nelotanserin and the psychedelic is MBDB, wherein the nelotanserin is administered to post-treat at least 330 minutes post to the MBDB. In some embodiments, the serotonin receptor modulator is nelotanserin and the psychedelic is MBDB, wherein the nelotanserin is administered to post-treat at least 360 minutes post to the MBDB. In some preferred embodiments, the serotonin receptor modulator is nelotanserin and the psychedelic is MBDB, wherein nelotanserin is administered to post-treat between about 60 minutes and about 180 minutes post to the administration of MBDB.

In some embodiments, the serotonin receptor modulator is pruvanserin and the psychedelic is MBDB, wherein the pruvanserin is administered to post-treat at least 15 minutes post to the administration of MBDB. In some embodiments, the serotonin receptor modulator is pruvanserin and the psychedelic is MBDB, wherein the pruvanserin is administered to post-treat at least 30 minutes post to the MBDB. In some embodiments, the serotonin receptor modulator is pruvanserin and the psychedelic is MBDB, wherein the pruvanserin is administered to post-treat at least 60 minutes post to the MBDB. In some embodiments, the serotonin receptor modulator is pruvanserin and the psychedelic is MBDB, wherein the pruvanserin is administered to post-treat between at least 60 minutes and 240 minutes post to the administration or release of MBDB. In some embodiments, the serotonin receptor modulator is pruvanserin and the psychedelic is MBDB, wherein the pruvanserin is administered to post-treat at least 90 minutes post to the MBDB. In some embodiments, the serotonin receptor modulator is pruvanserin and the psychedelic is MBDB, wherein the pruvanserin is administered to post-treat at least 120 minutes post to the MBDB. In some embodiments, the serotonin receptor modulator is pruvanserin and the psychedelic is MBDB, wherein the pruvanserin is administered to post-treat at least 150 minutes post to the MBDB. In some embodiments, the serotonin receptor modulator is pruvanserin and the psychedelic is MBDB, wherein the pruvanserin is administered to post-treat between about 15 minutes and about 150 minutes post to the MBDB. In some embodiments, the serotonin receptor modulator is pruvanserin and the psychedelic is MBDB, wherein the pruvanserin is administered to post-treat at least 180 minutes post to the MBDB. In some embodiments, the serotonin receptor modulator is pruvanserin and the psychedelic is MBDB, wherein the pruvanserin is administered to post-treat at least 210 minutes post to the MBDB.

In some embodiments, the serotonin receptor modulator is pruvanserin and the psychedelic is MBDB, wherein the pruvanserin is administered to post-treat at least 240 minutes post to the MBDB. In some embodiments, the serotonin receptor modulator is pruvanserin and the psychedelic is MBDB, wherein the pruvanserin is administered to post-treat at least 270 minutes post to the MBDB. In some embodiments, the serotonin receptor modulator is pruvanserin and the psychedelic is MBDB, wherein the pruvanserin is administered to post-treat at least 300 minutes post to the MBDB. In some embodiments, the serotonin receptor modulator is pruvanserin and the psychedelic is MBDB, wherein the pruvanserin is administered to post-treat at least 330 minutes post to the MBDB. In some embodiments, the serotonin receptor modulator is pruvanserin and the psychedelic is MBDB, wherein the pruvanserin is administered to post-treat at least 360 minutes post to the MBDB. In some preferred embodiments, the serotonin receptor modulator is pruvanserin and the psychedelic is MBDB, wherein pruvanserin is administered to post-treat between about 60 minutes and about 180 minutes post to the administration of MBDB.

In some embodiments, the serotonin receptor modulator is flibanserin and the psychedelic is MBDB, wherein the flibanserin is administered to post-treat at least 15 minutes post to the administration of MBDB. In some embodiments, the serotonin receptor modulator is flibanserin and the psychedelic is MBDB, wherein the flibanserin is administered to post-treat at least 30 minutes post to the MBDB. In some embodiments, the serotonin receptor modulator is flibanserin and the psychedelic is MBDB, wherein the flibanserin is administered to post-treat at least 60 minutes post to the MBDB. In some embodiments, the serotonin receptor modulator is flibanserin and the psychedelic is MBDB, wherein the flibanserin is administered to post-treat at least 90 minutes post to the MBDB. In some embodiments, the serotonin receptor modulator is flibanserin and the psychedelic is MBDB, wherein the flibanserin is administered to post-treat at least 120 minutes post to the MBDB. In some embodiments, the serotonin receptor modulator is flibanserin and the psychedelic is MBDB, wherein the flibanserin is administered to post-treat at least 150 minutes post to the MBDB. In some embodiments, the serotonin receptor modulator is flibanserin and the psychedelic is MBDB, wherein the flibanserin is administered to post-treat between about 15 minutes and about 150 minutes post to the MBDB. In some embodiments, the serotonin receptor modulator is flibanserin and the psychedelic is MBDB, wherein the flibanserin is administered to post-treat at least 180 minutes post to the MBDB. In some embodiments, the serotonin receptor modulator is flibanserin and the psychedelic is MBDB, wherein the flibanserin is administered to post-treat at least 210 minutes post to the MBDB. In some embodiments, the serotonin receptor modulator is flibanserin and the psychedelic is MBDB, wherein the flibanserin is administered to post-treat at least 240 minutes post to the MBDB. In some embodiments, the serotonin receptor modulator is flibanserin and the psychedelic is MBDB, wherein the flibanserin is administered to post-treat at least 270 minutes post to the MBDB. In some embodiments, the serotonin receptor modulator is flibanserin and the psychedelic is MBDB, wherein the flibanserin is administered to post-treat at least 300 minutes post to the MBDB. In some embodiments, the serotonin receptor modulator is flibanserin and the psychedelic is MBDB, wherein the flibanserin is administered to post-treat at least 330 minutes post to the MBDB. In some embodiments, the serotonin receptor modulator is flibanserin and the psychedelic is MBDB, wherein the flibanserin is administered to post-treat at least 360 minutes post to the MBDB. In some preferred embodiments, the serotonin receptor modulator is flibanserin and the psychedelic is MBDB, wherein flibanserin is administered to post-treat between about 60 minutes and about 180 minutes post to the administration of MBDB.

In some embodiments, the serotonin receptor modulator is olanzapine and the psychedelic is MBDB, wherein the olanzapine is administered to post-treat at least 15 minutes post to the administration of MBDB. In some embodiments, the serotonin receptor modulator is olanzapine and the psychedelic is MBDB, wherein the olanzapine is administered to post-treat at least 30 minutes post to the MBDB. In some embodiments, the serotonin receptor modulator is olanzapine and the psychedelic is MBDB, wherein the olanzapine is administered to post-treat at least 60 minutes post to the MBDB. In some embodiments, the serotonin receptor modulator is olanzapine and the psychedelic is MBDB, wherein the olanzapine is administered to post-treat at least 90 minutes post to the MBDB. In some embodiments, the serotonin receptor modulator is olanzapine and the psychedelic is MBDB, wherein the olanzapine is administered to post-treat at least 120 minutes post to the MBDB. In some embodiments, the serotonin receptor modulator is olanzapine and the psychedelic is MBDB, wherein the olanzapine is administered to post-treat at least 150 minutes post to the MBDB. In some embodiments, the serotonin receptor modulator is olanzapine and the psychedelic is MBDB, wherein the olanzapine is administered to post-treat between about 15 minutes and about 150 minutes post to the MBDB. In some embodiments, the serotonin receptor modulator is olanzapine and the psychedelic is MBDB, wherein the olanzapine is administered to post-treat at least 180 minutes post to the MBDB. In some embodiments, the serotonin receptor modulator is olanzapine and the psychedelic is MBDB, wherein the olanzapine is administered to post-treat at least 210 minutes post to the MBDB. In some embodiments, the serotonin receptor modulator is olanzapine and the psychedelic is MBDB, wherein the olanzapine is administered to post-treat at least 240 minutes post to the MBDB. In some embodiments, the serotonin receptor modulator is olanzapine and the psychedelic is MBDB, wherein the olanzapine is administered to post-treat at least 270 minutes post to the MBDB. In some embodiments, the serotonin receptor modulator is olanzapine and the psychedelic is MBDB, wherein the olanzapine is administered to post-treat at least 300 minutes post to the MBDB. In some embodiments, the serotonin receptor modulator is olanzapine and the psychedelic is MBDB, wherein the olanzapine is administered to post-treat at least 330 minutes post to the MBDB. In some embodiments, the serotonin receptor modulator is olanzapine and the psychedelic is MBDB, wherein the olanzapine is administered to post-treat at least 360 minutes post to the MBDB. In some preferred embodiments, the serotonin receptor modulator is olanzapine and the psychedelic is MBDB, wherein olanzapine is administered to post-treat between about 60 minutes and about 180 minutes post to the administration of MBDB.

In some embodiments, the serotonin receptor modulator is quetiapine and the psychedelic is MBDB, wherein the quetiapine is administered to post-treat at least 15 minutes post to the administration of MBDB. In some embodiments, the serotonin receptor modulator is quetiapine and the psychedelic is MBDB, wherein the quetiapine is administered to post-treat at least 30 minutes post to the MBDB. In some embodiments, the serotonin receptor modulator is quetiapine and the psychedelic is MBDB, wherein the quetiapine is administered to post-treat at least 60 minutes post to the MBDB. In some embodiments, the serotonin receptor modulator is quetiapine and the psychedelic is MBDB, wherein the quetiapine is administered to post-treat at least 90 minutes post to the MBDB. In some embodiments, the serotonin receptor modulator is quetiapine and the psychedelic is MBDB, wherein the quetiapine is administered to post-treat at least 120 minutes post to the MBDB. In some embodiments, the serotonin receptor modulator is quetiapine and the psychedelic is MBDB, wherein the quetiapine is administered to post-treat at least 150 minutes post to the MBDB. In some embodiments, the serotonin receptor modulator is quetiapine and the psychedelic is MBDB, wherein the quetiapine is administered to post-treat between about 15 minutes and about 150 minutes post to the MBDB. In some embodiments, the serotonin receptor modulator is quetiapine and the psychedelic is MBDB, wherein the quetiapine is administered to post-treat at least 180 minutes post to the MBDB. In some embodiments, the serotonin receptor modulator is quetiapine and the psychedelic is MBDB, wherein the quetiapine is administered to post-treat at least 210 minutes post to the MBDB. In some embodiments, the serotonin receptor modulator is quetiapine and the psychedelic is MBDB, wherein the quetiapine is administered to post-treat at least 240 minutes post to the MBDB. In some embodiments, the serotonin receptor modulator is quetiapine and the psychedelic is MBDB, wherein the quetiapine is administered to post-treat at least 270 minutes post to the MBDB. In some embodiments, the serotonin receptor modulator is quetiapine and the psychedelic is MBDB, wherein the quetiapine is administered to post-treat at least 300 minutes post to the MBDB. In some embodiments, the serotonin receptor modulator is quetiapine and the psychedelic is MBDB, wherein the quetiapine is administered to post-treat at least 330 minutes post to the MBDB. In some embodiments, the serotonin receptor modulator is quetiapine and the psychedelic is MBDB, wherein the quetiapine is administered to post-treat at least 360 minutes post to the MBDB. In some preferred embodiments, the serotonin receptor modulator is quetiapine and the psychedelic is MBDB, wherein quetiapine is administered to post-treat between about 60 minutes and about 180 minutes post to the administration of MBDB.

In some embodiments, the serotonin receptor modulator is risperidone and the psychedelic is MBDB, wherein the risperidone is administered to post-treat at least 15 minutes post to the administration of MBDB. In some embodiments, the serotonin receptor modulator is risperidone and the psychedelic is MBDB, wherein the risperidone is administered to post-treat at least 30 minutes post to the MBDB. In some embodiments, the serotonin receptor modulator is risperidone and the psychedelic is MBDB, wherein the risperidone is administered to post-treat at least 60 minutes post to the MBDB. In some embodiments, the serotonin receptor modulator is risperidone and the psychedelic is MBDB, wherein the risperidone is administered to post-treat at least 90 minutes post to the MBDB. In some embodiments, the serotonin receptor modulator is risperidone and the psychedelic is MBDB, wherein the risperidone is administered to post-treat at least 120 minutes post to the MBDB. In some embodiments, the serotonin receptor modulator is risperidone and the psychedelic is MBDB, wherein the risperidone is administered to post-treat at least 150 minutes post to the MBDB. In some embodiments, the serotonin receptor modulator is risperidone and the psychedelic is MBDB, wherein the risperidone is administered to post-treat between about 15 minutes and about 150 minutes post to the MBDB. In some embodiments, the serotonin receptor modulator is risperidone and the psychedelic is MBDB, wherein the risperidone is administered to post-treat at least 180 minutes post to the MBDB. In some embodiments, the serotonin receptor modulator is risperidone and the psychedelic is MBDB, wherein the risperidone is administered to post-treat at least 210 minutes post to the MBDB. In some embodiments, the serotonin receptor modulator is risperidone and the psychedelic is MBDB, wherein the risperidone is administered to post-treat at least 240 minutes post to the MBDB. In some embodiments, the serotonin receptor modulator is risperidone and the psychedelic is MBDB, wherein the risperidone is administered to post-treat at least 270 minutes post to the MBDB. In some embodiments, the serotonin receptor modulator is risperidone and the psychedelic is MBDB, wherein the risperidone is administered to post-treat at least 300 minutes post to the MBDB. In some embodiments, the serotonin receptor modulator is risperidone and the psychedelic is MBDB, wherein the risperidone is administered to post-treat at least 330 minutes post to the MBDB. In some embodiments, the serotonin receptor modulator is risperidone and the psychedelic is MBDB, wherein the risperidone is administered to post-treat at least 360 minutes post to the MBDB. In some preferred embodiments, the serotonin receptor modulator is risperidone and the psychedelic is MBDB, wherein risperidone is administered to post-treat between about 60 minutes and about 180 minutes post to the administration of MBDB.

In some embodiments, the serotonin receptor modulator is eplivanserin and the psychedelic is methylone, wherein the eplivanserin is administered to post-treat at least 15 minutes post to the administration of methylone. In some embodiments, the serotonin receptor modulator is eplivanserin and the psychedelic is methylone, wherein the eplivanserin is administered to post-treat at least 30 minutes post to the methylone. In some embodiments, the serotonin receptor modulator is eplivanserin and the psychedelic is methylone, wherein the eplivanserin is administered to post-treat at least 60 minutes post to the methylone. In some embodiments, the serotonin receptor modulator is eplivanserin and the psychedelic is methylone, wherein the eplivanserin is administered to post-treat at least 90 minutes post to the methylone. In some embodiments, the serotonin receptor modulator is eplivanserin and the psychedelic is methylone, wherein the eplivanserin is administered to post-treat between at least 30 minutes post and 360 minutes post to the release or administration of the methylone. In some embodiments, the serotonin receptor modulator is eplivanserin and the psychedelic is methylone, wherein the eplivanserin is administered to post-treat between at least 60 minutes post and 360 minutes post to the release or administration the methylone. In some embodiments, the serotonin receptor modulator is eplivanserin and the psychedelic is methylone, wherein the eplivanserin is administered to post-treat between at least 90 minutes and 240 minutes post to the methylone. In some embodiments, the serotonin receptor modulator is eplivanserin and the psychedelic is methylone, wherein the eplivanserin is administered to post-treat at least 120 minutes post to the methylone. In some embodiments, the serotonin receptor modulator is eplivanserin and the psychedelic is methylone, wherein the eplivanserin is administered to post-treat at least 150 minutes post to the methylone. In some embodiments, the serotonin receptor modulator is eplivanserin and the psychedelic is methylone, wherein the eplivanserin is administered to post-treat between about 15 minutes and about 150 minutes post to the methylone. In some embodiments, the serotonin receptor modulator is eplivanserin and the psychedelic is methylone, wherein the eplivanserin is administered to post-treat at least 180 minutes post to the methylone. In some embodiments, the serotonin receptor modulator is eplivanserin and the psychedelic is methylone, wherein the eplivanserin is administered to post-treat at least 210 minutes post to the methylone. In some embodiments, the serotonin receptor modulator is eplivanserin and the psychedelic is methylone, wherein the eplivanserin is administered to post-treat at least 240 minutes post to the methylone.

In some embodiments, the serotonin receptor modulator is eplivanserin and the psychedelic is methylone, wherein the eplivanserin is administered to post-treat at least 270 minutes post to the methylone. In some embodiments, the serotonin receptor modulator is eplivanserin and the psychedelic is methylone, wherein the eplivanserin is administered to post-treat at least 300 minutes post to the methylone. In some embodiments, the serotonin receptor modulator is eplivanserin and the psychedelic is methylone, wherein the eplivanserin is administered to post-treat at least 330 minutes post to the methylone. In some embodiments, the serotonin receptor modulator is eplivanserin and the psychedelic is methylone, wherein the eplivanserin is administered to post-treat at least 360 minutes post to the methylone.

In some preferred embodiments, the serotonin receptor modulator is eplivanserin and the psychedelic is methylone, wherein eplivanserin is administered to post-treat between about 60 minutes and about 180 minutes post to the administration of methylone.

In some embodiments, the serotonin receptor modulator is volinanserin and the psychedelic is methylone, wherein the volinanserin is administered to post-treat at least 15 minutes post to the administration of methylone. In some embodiments, the serotonin receptor modulator is volinanserin and the psychedelic is methylone, wherein the volinanserin is administered to post-treat at least 30 minutes post to the methylone. In some embodiments, the serotonin receptor modulator is volinanserin and the psychedelic is methylone, wherein the volinanserin is administered to post-treat at least 60 minutes post to the methylone. In some embodiments, the serotonin receptor modulator is volinanserin and the psychedelic is methylone, wherein the volinanserin is administered to post-treat a subject between at least 15 minutes and 360 minutes post to the administration or release of methylone. In some embodiments, the serotonin receptor modulator is volinanserin and the psychedelic is methylone, wherein the volinanserin is administered to post-treat between at least 30 minutes and 360 minutes post to the administration or release of methylone. In some embodiments, the serotonin receptor modulator is volinanserin and the psychedelic is methylone, wherein the volinanserin is administered to post-treat between at least 60 minutes and 240 minutes post to the administration or release of methylone. In some embodiments, the serotonin receptor modulator is volinanserin and the psychedelic is methylone, wherein the volinanserin is administered to post-treat at least 90 minutes post to the methylone. In some embodiments, the serotonin receptor modulator is volinanserin and the psychedelic is methylone, wherein the volinanserin is administered to post-treat at least 120 minutes post to the methylone.

In some embodiments, the serotonin receptor modulator is volinanserin and the psychedelic is methylone, wherein the volinanserin is administered to post-treat at least 150 minutes post to the methylone. In some embodiments, the serotonin receptor modulator is volinanserin and the psychedelic is methylone, wherein the volinanserin is administered to post-treat between about 15 minutes and about 150 minutes post to the methylone. In some embodiments, the serotonin receptor modulator is volinanserin and the psychedelic is methylone, wherein the volinanserin is administered to post-treat at least 180 minutes post to the methylone. In some embodiments, the serotonin receptor modulator is volinanserin and the psychedelic is methylone, wherein the volinanserin is administered to post-treat at least 210 minutes post to the methylone. In some embodiments, the serotonin receptor modulator is volinanserin and the psychedelic is methylone, wherein the volinanserin is administered to post-treat at least 240 minutes post to the methylone. In some embodiments, the serotonin receptor modulator is volinanserin and the psychedelic is methylone, wherein the volinanserin is administered to post-treat at least 270 minutes post to the methylone. In some embodiments, the serotonin receptor modulator is volinanserin and the psychedelic is methylone, wherein the volinanserin is administered to post-treat at least 300 minutes post to the methylone. In some embodiments, the serotonin receptor modulator is volinanserin and the psychedelic is methylone, wherein the volinanserin is administered to post-treat at least 330 minutes post to the methylone. In some embodiments, the serotonin receptor modulator is volinanserin and the psychedelic is methylone, wherein the volinanserin is administered to post-treat at least 360 minutes post to the methylone. In some preferred embodiments, the serotonin receptor modulator is volinanserin and the psychedelic is methylone, wherein volinanserin is administered to post-treat between about 60 minutes and about 180 minutes post to the administration of methylone.

In some embodiments, the serotonin receptor modulator is ketanserin and the psychedelic is methylone, wherein the ketanserin is administered to post-treat at least 15 minutes post to the administration of methylone. In some embodiments, the serotonin receptor modulator is ketanserin and the psychedelic is methylone, wherein the ketanserin is administered to post-treat at least 30 minutes post to the methylone. In some embodiments, the serotonin receptor modulator is ketanserin and the psychedelic is methylone, wherein the ketanserin is administered to post-treat at least 60 minutes post to the methylone. In some embodiments, the serotonin receptor modulator is ketanserin and the psychedelic is methylone, wherein the ketanserin is administered to post-treat between at least 30 minutes and 360 minutes post to the administration or release methylone. In some embodiments, the serotonin receptor modulator is ketanserin and the psychedelic is methylone, wherein the ketanserin is administered to post-treat between at least 60 minutes and 240 minutes post to the administration or release of methylone. In some embodiments, the serotonin receptor modulator is ketanserin and the psychedelic is methylone, wherein the ketanserin is administered to post-treat at least 90 minutes post to the methylone. In some embodiments, the serotonin receptor modulator is ketanserin and the psychedelic is methylone, wherein the ketanserin is administered to post-treat at least 120 minutes post to the methylone. In some embodiments, the serotonin receptor modulator is ketanserin and the psychedelic is methylone, wherein the ketanserin is administered to post-treat at least 150 minutes post to the methylone. In some embodiments, the serotonin receptor modulator is ketanserin and the psychedelic is methylone, wherein the ketanserin is administered to post-treat between about 15 minutes and about 150 minutes post to the methylone. In some embodiments, the serotonin receptor modulator is ketanserin and the psychedelic is methylone, wherein the ketanserin is administered to post-treat at least 180 minutes post to the methylone. In some embodiments, the serotonin receptor modulator is ketanserin and the psychedelic is methylone, wherein the ketanserin is administered to post-treat at least 210 minutes post to the methylone. In some embodiments, the serotonin receptor modulator is ketanserin and the psychedelic is methylone, wherein the ketanserin is administered to post-treat at least 240 minutes post to the methylone. In some embodiments, the serotonin receptor modulator is ketanserin and the psychedelic is methylone, wherein the ketanserin is administered to post-treat at least 270 minutes post to the methylone. In some embodiments, the serotonin receptor modulator is ketanserin and the psychedelic is methylone, wherein the ketanserin is administered to post-treat at least 300 minutes post to the methylone. In some embodiments, the serotonin receptor modulator is ketanserin and the psychedelic is methylone, wherein the ketanserin is administered to post-treat at least 330 minutes post to the methylone. In some embodiments, the serotonin receptor modulator is ketanserin and the psychedelic is methylone, wherein the ketanserin is administered to post-treat at least 360 minutes post to the methylone. In some preferred embodiments, the serotonin receptor modulator is ketanserin and the psychedelic is methylone, wherein ketanserin is administered to post-treat between about 60 minutes and about 180 minutes post to the administration of methylone.

In some embodiments, the serotonin receptor modulator is ritanserin and the psychedelic is methylone, wherein the ritanserin is administered to post-treat at least 15 minutes post to the administration of methylone. In some embodiments, the serotonin receptor modulator is ritanserin and the psychedelic is methylone, wherein the ritanserin is administered to post-treat at least 30 minutes post to the methylone. In some embodiments, the serotonin receptor modulator is ritanserin and the psychedelic is methylone, wherein the ritanserin is administered to post-treat at least 60 minutes post to the methylone. In some embodiments, the serotonin receptor modulator is ritanserin and the psychedelic is methylone, wherein the ritanserin is administered to post-treat between at least 60 minutes and 240 minutes post to the administration or release of methylone. In some embodiments, the serotonin receptor modulator is ritanserin and the psychedelic is methylone, wherein the ritanserin is administered to post-treat at least 90 minutes post to the methylone. In some embodiments, the serotonin receptor modulator is ritanserin and the psychedelic is methylone, wherein the ritanserin is administered to post-treat at least 120 minutes post to the methylone. In some embodiments, the serotonin receptor modulator is ritanserin and the psychedelic is methylone, wherein the ritanserin is administered to post-treat at least 150 minutes post to the methylone. In some embodiments, the serotonin receptor modulator is ritanserin and the psychedelic is methylone, wherein the ritanserin is administered to post-treat between about 15 minutes and about 150 minutes post to the methylone. In some embodiments, the serotonin receptor modulator is ritanserin and the psychedelic is methylone, wherein the ritanserin is administered to post-treat at least 180 minutes post to the methylone. In some embodiments, the serotonin receptor modulator is ritanserin and the psychedelic is methylone, wherein the ritanserin is administered to post-treat at least 210 minutes post to the methylone. In some embodiments, the serotonin receptor modulator is ritanserin and the psychedelic is methylone, wherein the ritanserin is administered to post-treat at least 240 minutes post to the methylone. In some embodiments, the serotonin receptor modulator is ritanserin and the psychedelic is methylone, wherein the ritanserin is administered to post-treat at least 270 minutes post to the methylone. In some embodiments, the serotonin receptor modulator is ritanserin and the psychedelic is methylone, wherein the ritanserin is administered to post-treat at least 300 minutes post to the methylone. In some embodiments, the serotonin receptor modulator is ritanserin and the psychedelic is methylone, wherein the ritanserin is administered to post-treat at least 330 minutes post to the methylone. In some embodiments, the serotonin receptor modulator is ritanserin and the psychedelic is methylone, wherein the ritanserin is administered to post-treat at least 360 minutes post to the methylone. In some preferred embodiments, the serotonin receptor modulator is ritanserin and the psychedelic is methylone, wherein ritanserin is administered to post-treat between about 60 minutes and about 180 minutes post to the administration of methylone.

In some embodiments, the serotonin receptor modulator is pimavanserin and the psychedelic is methylone, wherein the pimavanserin is administered to post-treat at least 15 minutes post to the administration of methylone. In some embodiments, the serotonin receptor modulator is pimavanserin and the psychedelic is methylone, wherein the pimavanserin is administered to post-treat at least 30 minutes post to the methylone. In some embodiments, the serotonin receptor modulator is pimavanserin and the psychedelic is methylone, wherein the pimavanserin is administered to post-treat at least 60 minutes post to the methylone. In some embodiments, the serotonin receptor modulator is pimavanserin and the psychedelic is methylone, wherein the pimavanserin is administered to post-treat between at least 60 minutes and 240 minutes post to the administration or release of methylone. In some embodiments, the serotonin receptor modulator is pimavanserin and the psychedelic is methylone, wherein the pimavanserin is administered to post-treat at least 90 minutes post to the methylone. In some embodiments, the serotonin receptor modulator is pimavanserin and the psychedelic is methylone, wherein the pimavanserin is administered to post-treat at least 120 minutes post to the methylone. In some embodiments, the serotonin receptor modulator is pimavanserin and the psychedelic is methylone, wherein the pimavanserin is administered to post-treat at least 150 minutes post to the methylone. In some embodiments, the serotonin receptor modulator is pimavanserin and the psychedelic is methylone, wherein the pimavanserin is administered to post-treat between about 15 minutes and about 150 minutes post to the methylone. In some embodiments, the serotonin receptor modulator is pimavanserin and the psychedelic is methylone, wherein the pimavanserin is administered to post-treat at least 180 minutes post to the methylone. In some embodiments, the serotonin receptor modulator is pimavanserin and the psychedelic is methylone, wherein the pimavanserin is administered to post-treat at least 210 minutes post to the methylone. In some embodiments, the serotonin receptor modulator is pimavanserin and the psychedelic is methylone, wherein the pimavanserin is administered to post-treat at least 240 minutes post to the methylone. In some embodiments, the serotonin receptor modulator is pimavanserin and the psychedelic is methylone, wherein the pimavanserin is administered to post-treat at least 270 minutes post to the methylone. In some embodiments, the serotonin receptor modulator is pimavanserin and the psychedelic is methylone, wherein the pimavanserin is administered to post-treat at least 300 minutes post to the methylone. In some embodiments, the serotonin receptor modulator is pimavanserin and the psychedelic is methylone, wherein the pimavanserin is administered to post-treat at least 330 minutes post to the methylone. In some embodiments, the serotonin receptor modulator is pimavanserin and the psychedelic is methylone, wherein the pimavanserin is administered to post-treat at least 360 minutes post to the methylone. In some preferred embodiments, the serotonin receptor modulator is pimavanserin and the psychedelic is methylone, wherein pimavanserin is administered to post-treat between about 60 minutes and about 180 minutes post to the administration of methylone.

In some embodiments, the serotonin receptor modulator is nelotanserin and the psychedelic is methylone, wherein the nelotanserin is administered to post-treat at least 15 minutes post to the administration of methylone. In some embodiments, the serotonin receptor modulator is nelotanserin and the psychedelic is methylone, wherein the nelotanserin is administered to post-treat at least 30 minutes post to the methylone. In some embodiments, the serotonin receptor modulator is nelotanserin and the psychedelic is methylone, wherein the nelotanserin is administered to post-treat at least 60 minutes post to the methylone. In some embodiments, the serotonin receptor modulator is nelotanserin and the psychedelic is methylone, wherein the nelotanserin is administered to post-treat between at least 60 minutes and 240 minutes post to the administration or release of methylone. In some embodiments, the serotonin receptor modulator is nelotanserin and the psychedelic is methylone, wherein the nelotanserin is administered to post-treat at least 90 minutes post to the methylone. In some embodiments, the serotonin receptor modulator is nelotanserin and the psychedelic is methylone, wherein the nelotanserin is administered to post-treat at least 120 minutes post to the methylone.

In some embodiments, the serotonin receptor modulator is nelotanserin and the psychedelic is methylone, wherein the nelotanserin is administered to post-treat at least 150 minutes post to the methylone. In some embodiments, the serotonin receptor modulator is nelotanserin and the psychedelic is methylone, wherein the nelotanserin is administered to post-treat between about 15 minutes and about 150 minutes post to the methylone. In some embodiments, the serotonin receptor modulator is nelotanserin and the psychedelic is methylone, wherein the nelotanserin is administered to post-treat at least 180 minutes post to the methylone. In some embodiments, the serotonin receptor modulator is nelotanserin and the psychedelic is methylone, wherein the nelotanserin is administered to post-treat at least 210 minutes post to the methylone. In some embodiments, the serotonin receptor modulator is nelotanserin and the psychedelic is methylone, wherein the nelotanserin is administered to post-treat at least 240 minutes post to the methylone. In some embodiments, the serotonin receptor modulator is nelotanserin and the psychedelic is methylone, wherein the nelotanserin is administered to post-treat at least 270 minutes post to the methylone.

In some embodiments, the serotonin receptor modulator is nelotanserin and the psychedelic is methylone, wherein the nelotanserin is administered to post-treat at least 300 minutes post to the methylone. In some embodiments, the serotonin receptor modulator is nelotanserin and the psychedelic is methylone, wherein the nelotanserin is administered to post-treat at least 330 minutes post to the methylone. In some embodiments, the serotonin receptor modulator is nelotanserin and the psychedelic is methylone, wherein the nelotanserin is administered to post-treat at least 360 minutes post to the methylone. In some preferred embodiments, the serotonin receptor modulator is nelotanserin and the psychedelic is methylone, wherein nelotanserin is administered to post-treat between about 60 minutes and about 180 minutes post to the administration of methylone.

In some embodiments, the serotonin receptor modulator is pruvanserin and the psychedelic is methylone, wherein the pruvanserin is administered to post-treat at least 15 minutes post to the administration of methylone. In some embodiments, the serotonin receptor modulator is pruvanserin and the psychedelic is methylone, wherein the pruvanserin is administered to post-treat at least 30 minutes post to the methylone. In some embodiments, the serotonin receptor modulator is pruvanserin and the psychedelic is methylone, wherein the pruvanserin is administered to post-treat at least 60 minutes post to the methylone. In some embodiments, the serotonin receptor modulator is pruvanserin and the psychedelic is methylone, wherein the pruvanserin is administered to post-treat between at least 60 minutes and 240 minutes post to the administration or release of methylone. In some embodiments, the serotonin receptor modulator is pruvanserin and the psychedelic is methylone, wherein the pruvanserin is administered to post-treat at least 90 minutes post to the methylone. In some embodiments, the serotonin receptor modulator is pruvanserin and the psychedelic is methylone, wherein the pruvanserin is administered to post-treat at least 120 minutes post to the methylone. In some embodiments, the serotonin receptor modulator is pruvanserin and the psychedelic is methylone, wherein the pruvanserin is administered to post-treat at least 150 minutes post to the methylone. In some embodiments, the serotonin receptor modulator is pruvanserin and the psychedelic is methylone, wherein the pruvanserin is administered to post-treat between about 15 minutes and about 150 minutes post to the methylone. In some embodiments, the serotonin receptor modulator is pruvanserin and the psychedelic is methylone, wherein the pruvanserin is administered to post-treat at least 180 minutes post to the methylone. In some embodiments, the serotonin receptor modulator is pruvanserin and the psychedelic is methylone, wherein the pruvanserin is administered to post-treat at least 210 minutes post to the methylone.

In some embodiments, the serotonin receptor modulator is pruvanserin and the psychedelic is methylone, wherein the pruvanserin is administered to post-treat at least 240 minutes post to the methylone. In some embodiments, the serotonin receptor modulator is pruvanserin and the psychedelic is methylone, wherein the pruvanserin is administered to post-treat at least 270 minutes post to the methylone. In some embodiments, the serotonin receptor modulator is pruvanserin and the psychedelic is methylone, wherein the pruvanserin is administered to post-treat at least 300 minutes post to the methylone. In some embodiments, the serotonin receptor modulator is pruvanserin and the psychedelic is methylone, wherein the pruvanserin is administered to post-treat at least 330 minutes post to the methylone. In some embodiments, the serotonin receptor modulator is pruvanserin and the psychedelic is methylone, wherein the pruvanserin is administered to post-treat at least 360 minutes post to the methylone. In some preferred embodiments, the serotonin receptor modulator is pruvanserin and the psychedelic is methylone, wherein pruvanserin is administered to post-treat between about 60 minutes and about 180 minutes post to the administration of methylone.

In some embodiments, the serotonin receptor modulator is flibanserin and the psychedelic is methylone, wherein the flibanserin is administered to post-treat at least 15 minutes post to the administration of methylone. In some embodiments, the serotonin receptor modulator is flibanserin and the psychedelic is methylone, wherein the flibanserin is administered to post-treat at least 30 minutes post to the methylone. In some embodiments, the serotonin receptor modulator is flibanserin and the psychedelic is methylone, wherein the flibanserin is administered to post-treat at least 60 minutes post to the methylone. In some embodiments, the serotonin receptor modulator is flibanserin and the psychedelic is methylone, wherein the flibanserin is administered to post-treat at least 90 minutes post to the methylone. In some embodiments, the serotonin receptor modulator is flibanserin and the psychedelic is methylone, wherein the flibanserin is administered to post-treat at least 120 minutes post to the methylone. In some embodiments, the serotonin receptor modulator is flibanserin and the psychedelic is methylone, wherein the flibanserin is administered to post-treat at least 150 minutes post to the methylone. In some embodiments, the serotonin receptor modulator is flibanserin and the psychedelic is methylone, wherein the flibanserin is administered to post-treat between about 15 minutes and about 150 minutes post to the methylone. In some embodiments, the serotonin receptor modulator is flibanserin and the psychedelic is methylone, wherein the flibanserin is administered to post-treat at least 180 minutes post to the methylone. In some embodiments, the serotonin receptor modulator is flibanserin and the psychedelic is methylone, wherein the flibanserin is administered to post-treat at least 210 minutes post to the methylone. In some embodiments, the serotonin receptor modulator is flibanserin and the psychedelic is methylone, wherein the flibanserin is administered to post-treat at least 240 minutes post to the methylone. In some embodiments, the serotonin receptor modulator is flibanserin and the psychedelic is methylone, wherein the flibanserin is administered to post-treat at least 270 minutes post to the methylone. In some embodiments, the serotonin receptor modulator is flibanserin and the psychedelic is methylone, wherein the flibanserin is administered to post-treat at least 300 minutes post to the methylone. In some embodiments, the serotonin receptor modulator is flibanserin and the psychedelic is methylone, wherein the flibanserin is administered to post-treat at least 330 minutes post to the methylone. In some embodiments, the serotonin receptor modulator is flibanserin and the psychedelic is methylone, wherein the flibanserin is administered to post-treat at least 360 minutes post to the methylone. In some preferred embodiments, the serotonin receptor modulator is flibanserin and the psychedelic is methylone, wherein flibanserin is administered to post-treat between about 60 minutes and about 180 minutes post to the administration of methylone.

In some embodiments, the serotonin receptor modulator is olanzapine and the psychedelic is methylone, wherein the olanzapine is administered to post-treat at least 15 minutes post to the administration of methylone. In some embodiments, the serotonin receptor modulator is olanzapine and the psychedelic is methylone, wherein the olanzapine is administered to post-treat at least 30 minutes post to the methylone. In some embodiments, the serotonin receptor modulator is olanzapine and the psychedelic is methylone, wherein the olanzapine is administered to post-treat at least 60 minutes post to the methylone. In some embodiments, the serotonin receptor modulator is olanzapine and the psychedelic is methylone, wherein the olanzapine is administered to post-treat at least 90 minutes post to the methylone. In some embodiments, the serotonin receptor modulator is olanzapine and the psychedelic is methylone, wherein the olanzapine is administered to post-treat at least 120 minutes post to the methylone. In some embodiments, the serotonin receptor modulator is olanzapine and the psychedelic is methylone, wherein the olanzapine is administered to post-treat at least 150 minutes post to the methylone. In some embodiments, the serotonin receptor modulator is olanzapine and the psychedelic is methylone, wherein the olanzapine is administered to post-treat between about 15 minutes and about 150 minutes post to the methylone. In some embodiments, the serotonin receptor modulator is olanzapine and the psychedelic is methylone, wherein the olanzapine is administered to post-treat at least 180 minutes post to the methylone. In some embodiments, the serotonin receptor modulator is olanzapine and the psychedelic is methylone, wherein the olanzapine is administered to post-treat at least 210 minutes post to the methylone. In some embodiments, the serotonin receptor modulator is olanzapine and the psychedelic is methylone, wherein the olanzapine is administered to post-treat at least 240 minutes post to the methylone. In some embodiments, the serotonin receptor modulator is olanzapine and the psychedelic is methylone, wherein the olanzapine is administered to post-treat at least 270 minutes post to the methylone. In some embodiments, the serotonin receptor modulator is olanzapine and the psychedelic is methylone, wherein the olanzapine is administered to post-treat at least 300 minutes post to the methylone. In some embodiments, the serotonin receptor modulator is olanzapine and the psychedelic is methylone, wherein the olanzapine is administered to post-treat at least 330 minutes post to the methylone. In some embodiments, the serotonin receptor modulator is olanzapine and the psychedelic is methylone, wherein the olanzapine is administered to post-treat at least 360 minutes post to the methylone. In some preferred embodiments, the serotonin receptor modulator is olanzapine and the psychedelic is methylone, wherein olanzapine is administered to post-treat between about 60 minutes and about 180 minutes post to the administration of methylone.

In some embodiments, the serotonin receptor modulator is quetiapine and the psychedelic is methylone, wherein the quetiapine is administered to post-treat at least 15 minutes post to the administration of methylone. In some embodiments, the serotonin receptor modulator is quetiapine and the psychedelic is methylone, wherein the quetiapine is administered to post-treat at least 30 minutes post to the methylone. In some embodiments, the serotonin receptor modulator is quetiapine and the psychedelic is methylone, wherein the quetiapine is administered to post-treat at least 60 minutes post to the methylone. In some embodiments, the serotonin receptor modulator is quetiapine and the psychedelic is methylone, wherein the quetiapine is administered to post-treat at least 90 minutes post to the methylone. In some embodiments, the serotonin receptor modulator is quetiapine and the psychedelic is methylone, wherein the quetiapine is administered to post-treat at least 120 minutes post to the methylone. In some embodiments, the serotonin receptor modulator is quetiapine and the psychedelic is methylone, wherein the quetiapine is administered to post-treat at least 150 minutes post to the methylone. In some embodiments, the serotonin receptor modulator is quetiapine and the psychedelic is methylone, wherein the quetiapine is administered to post-treat between about 15 minutes and about 150 minutes post to the methylone. In some embodiments, the serotonin receptor modulator is quetiapine and the psychedelic is methylone, wherein the quetiapine is administered to post-treat at least 180 minutes post to the methylone. In some embodiments, the serotonin receptor modulator is quetiapine and the psychedelic is methylone, wherein the quetiapine is administered to post-treat at least 210 minutes post to the methylone. In some embodiments, the serotonin receptor modulator is quetiapine and the psychedelic is methylone, wherein the quetiapine is administered to post-treat at least 240 minutes post to the methylone. In some embodiments, the serotonin receptor modulator is quetiapine and the psychedelic is methylone, wherein the quetiapine is administered to post-treat at least 270 minutes post to the methylone. In some embodiments, the serotonin receptor modulator is quetiapine and the psychedelic is methylone, wherein the quetiapine is administered to post-treat at least 300 minutes post to the methylone. In some embodiments, the serotonin receptor modulator is quetiapine and the psychedelic is methylone, wherein the quetiapine is administered to post-treat at least 330 minutes post to the methylone. In some embodiments, the serotonin receptor modulator is quetiapine and the psychedelic is methylone, wherein the quetiapine is administered to post-treat at least 360 minutes post to the methylone. In some preferred embodiments, the serotonin receptor modulator is quetiapine and the psychedelic is methylone, wherein quetiapine is administered to post-treat between about 60 minutes and about 180 minutes post to the administration of methylone.

In some embodiments, the serotonin receptor modulator is risperidone and the psychedelic is methylone, wherein the risperidone is administered to post-treat at least 15 minutes post to the administration of methylone. In some embodiments, the serotonin receptor modulator is risperidone and the psychedelic is methylone, wherein the risperidone is administered to post-treat at least 30 minutes post to the methylone. In some embodiments, the serotonin receptor modulator is risperidone and the psychedelic is methylone, wherein the risperidone is administered to post-treat at least 60 minutes post to the methylone. In some embodiments, the serotonin receptor modulator is risperidone and the psychedelic is methylone, wherein the risperidone is administered to post-treat at least 90 minutes post to the methylone. In some embodiments, the serotonin receptor modulator is risperidone and the psychedelic is methylone, wherein the risperidone is administered to post-treat at least 120 minutes post to the methylone. In some embodiments, the serotonin receptor modulator is risperidone and the psychedelic is methylone, wherein the risperidone is administered to post-treat at least 150 minutes post to the methylone. In some embodiments, the serotonin receptor modulator is risperidone and the psychedelic is methylone, wherein the risperidone is administered to post-treat between about 15 minutes and about 150 minutes post to the methylone. In some embodiments, the serotonin receptor modulator is risperidone and the psychedelic is methylone, wherein the risperidone is administered to post-treat at least 180 minutes post to the methylone. In some embodiments, the serotonin receptor modulator is risperidone and the psychedelic is methylone, wherein the risperidone is administered to post-treat at least 210 minutes post to the methylone. In some embodiments, the serotonin receptor modulator is risperidone and the psychedelic is methylone, wherein the risperidone is administered to post-treat at least 240 minutes post to the methylone. In some embodiments, the serotonin receptor modulator is risperidone and the psychedelic is methylone, wherein the risperidone is administered to post-treat at least 270 minutes post to the methylone. In some embodiments, the serotonin receptor modulator is risperidone and the psychedelic is methylone, wherein the risperidone is administered to post-treat at least 300 minutes post to the methylone. In some embodiments, the serotonin receptor modulator is risperidone and the psychedelic is methylone, wherein the risperidone is administered to post-treat at least 330 minutes post to the methylone. In some embodiments, the serotonin receptor modulator is risperidone and the psychedelic is methylone, wherein the risperidone is administered to post-treat at least 360 minutes post to the methylone. In some preferred embodiments, the serotonin receptor modulator is risperidone and the psychedelic is methylone, wherein risperidone is administered to post-treat between about 60 minutes and about 180 minutes post to the administration of methylone.

In some embodiments, the serotonin receptor modulator is eplivanserin and the psychedelic is (R)-methylone, wherein the eplivanserin is administered to post-treat at least 15 minutes post to the administration of (R)-methylone. In some embodiments, the serotonin receptor modulator is eplivanserin and the psychedelic is (R)-methylone, wherein the eplivanserin is administered to post-treat at least 30 minutes post to the (R)-methylone. In some embodiments, the serotonin receptor modulator is eplivanserin and the psychedelic is (R)-methylone, wherein the eplivanserin is administered to post-treat at least 60 minutes post to the (R)-methylone. In some embodiments, the serotonin receptor modulator is eplivanserin and the psychedelic is (R)-methylone, wherein the eplivanserin is administered to post-treat at least 90 minutes post to the (R)-methylone. In some embodiments, the serotonin receptor modulator is eplivanserin and the psychedelic is (R)-methylone, wherein the eplivanserin is administered to post-treat between at least 30 minutes post and 360 minutes post to the release or administration of the (R)-methylone. In some embodiments, the serotonin receptor modulator is eplivanserin and the psychedelic is (R)-methylone, wherein the eplivanserin is administered to post-treat between at least 60 minutes post and 360 minutes post to the release or administration the (R)-methylone. In some embodiments, the serotonin receptor modulator is eplivanserin and the psychedelic is (R)-methylone, wherein the eplivanserin is administered to post-treat between at least 90 minutes and 240 minutes post to the (R)-methylone. In some embodiments, the serotonin receptor modulator is eplivanserin and the psychedelic is (R)-methylone, wherein the eplivanserin is administered to post-treat at least 120 minutes post to the (R)-methylone. In some embodiments, the serotonin receptor modulator is eplivanserin and the psychedelic is (R)-methylone, wherein the eplivanserin is administered to post-treat at least 150 minutes post to the (R)-methylone. In some embodiments, the serotonin receptor modulator is eplivanserin and the psychedelic is (R)-methylone, wherein the eplivanserin is administered to post-treat between about 15 minutes and about 150 minutes post to the (R)-methylone. In some embodiments, the serotonin receptor modulator is eplivanserin and the psychedelic is (R)-methylone, wherein the eplivanserin is administered to post-treat at least 180 minutes post to the (R)-methylone. In some embodiments, the serotonin receptor modulator is eplivanserin and the psychedelic is (R)-methylone, wherein the eplivanserin is administered to post-treat at least 210 minutes post to the (R)-methylone. In some embodiments, the serotonin receptor modulator is eplivanserin and the psychedelic is (R)-methylone, wherein the eplivanserin is administered to post-treat at least 240 minutes post to the (R)-methylone.

In some embodiments, the serotonin receptor modulator is eplivanserin and the psychedelic is (R)-methylone, wherein the eplivanserin is administered to post-treat at least 270 minutes post to the (R)-methylone. In some embodiments, the serotonin receptor modulator is eplivanserin and the psychedelic is (R)-methylone, wherein the eplivanserin is administered to post-treat at least 300 minutes post to the (R)-methylone. In some embodiments, the serotonin receptor modulator is eplivanserin and the psychedelic is (R)-methylone, wherein the eplivanserin is administered to post-treat at least 330 minutes post to the (R)-methylone. In some embodiments, the serotonin receptor modulator is eplivanserin and the psychedelic is (R)-methylone, wherein the eplivanserin is administered to post-treat at least 360 minutes post to the (R)-methylone.

In some preferred embodiments, the serotonin receptor modulator is eplivanserin and the psychedelic is (R)-methylone, wherein eplivanserin is administered to post-treat between about 60 minutes and about 180 minutes post to the administration of (R)-methylone.

In some embodiments, the serotonin receptor modulator is volinanserin and the psychedelic is (R)-methylone, wherein the volinanserin is administered to post-treat at least 15 minutes post to the administration of (R)-methylone. In some embodiments, the serotonin receptor modulator is volinanserin and the psychedelic is (R)-methylone, wherein the volinanserin is administered to post-treat at least 30 minutes post to the (R)-methylone. In some embodiments, the serotonin receptor modulator is volinanserin and the psychedelic is (R)-methylone, wherein the volinanserin is administered to post-treat at least 60 minutes post to the (R)-methylone. In some embodiments, the serotonin receptor modulator is volinanserin and the psychedelic is (R)-methylone, wherein the volinanserin is administered to post-treat a subject between at least 15 minutes and 360 minutes post to the administration or release of (R)-methylone. In some embodiments, the serotonin receptor modulator is volinanserin and the psychedelic is (R)-methylone, wherein the volinanserin is administered to post-treat between at least 30 minutes and 360 minutes post to the administration or release of (R)-methylone. In some embodiments, the serotonin receptor modulator is volinanserin and the psychedelic is (R)-methylone, wherein the volinanserin is administered to post-treat between at least 60 minutes and 240 minutes post to the administration or release of (R)-methylone. In some embodiments, the serotonin receptor modulator is volinanserin and the psychedelic is (R)-methylone, wherein the volinanserin is administered to post-treat at least 90 minutes post to the (R)-methylone. In some embodiments, the serotonin receptor modulator is volinanserin and the psychedelic is (R)-methylone, wherein the volinanserin is administered to post-treat at least 120 minutes post to the (R)-methylone.

In some embodiments, the serotonin receptor modulator is volinanserin and the psychedelic is (R)-methylone, wherein the volinanserin is administered to post-treat at least 150 minutes post to the (R)-methylone. In some embodiments, the serotonin receptor modulator is volinanserin and the psychedelic is (R)-methylone, wherein the volinanserin is administered to post-treat between about 15 minutes and about 150 minutes post to the (R)-methylone. In some embodiments, the serotonin receptor modulator is volinanserin and the psychedelic is (R)-methylone, wherein the volinanserin is administered to post-treat at least 180 minutes post to the (R)-methylone. In some embodiments, the serotonin receptor modulator is volinanserin and the psychedelic is (R)-methylone, wherein the volinanserin is administered to post-treat at least 210 minutes post to the (R)-methylone. In some embodiments, the serotonin receptor modulator is volinanserin and the psychedelic is (R)-methylone, wherein the volinanserin is administered to post-treat at least 240 minutes post to the (R)-methylone. In some embodiments, the serotonin receptor modulator is volinanserin and the psychedelic is (R)-methylone, wherein the volinanserin is administered to post-treat at least 270 minutes post to the (R)-methylone. In some embodiments, the serotonin receptor modulator is volinanserin and the psychedelic is (R)-methylone, wherein the volinanserin is administered to post-treat at least 300 minutes post to the (R)-methylone. In some embodiments, the serotonin receptor modulator is volinanserin and the psychedelic is (R)-methylone, wherein the volinanserin is administered to post-treat at least 330 minutes post to the (R)-methylone. In some embodiments, the serotonin receptor modulator is volinanserin and the psychedelic is (R)-methylone, wherein the volinanserin is administered to post-treat at least 360 minutes post to the (R)-methylone. In some preferred embodiments, the serotonin receptor modulator is volinanserin and the psychedelic is (R)-methylone, wherein volinanserin is administered to post-treat between about 60 minutes and about 180 minutes post to the administration of (R)-methylone.

In some embodiments, the serotonin receptor modulator is ketanserin and the psychedelic is (R)-methylone, wherein the ketanserin is administered to post-treat at least 15 minutes post to the administration of (R)-methylone. In some embodiments, the serotonin receptor modulator is ketanserin and the psychedelic is (R)-methylone, wherein the ketanserin is administered to post-treat at least 30 minutes post to the (R)-methylone. In some embodiments, the serotonin receptor modulator is ketanserin and the psychedelic is (R)-methylone, wherein the ketanserin is administered to post-treat at least 60 minutes post to the (R)-methylone. In some embodiments, the serotonin receptor modulator is ketanserin and the psychedelic is (R)-methylone, wherein the ketanserin is administered to post-treat between at least 30 minutes and 360 minutes post to the administration or release (R)-methylone. In some embodiments, the serotonin receptor modulator is ketanserin and the psychedelic is (R)-methylone, wherein the ketanserin is administered to post-treat between at least 60 minutes and 240 minutes post to the administration or release of (R)-methylone. In some embodiments, the serotonin receptor modulator is ketanserin and the psychedelic is (R)-methylone, wherein the ketanserin is administered to post-treat at least 90 minutes post to the (R)-methylone. In some embodiments, the serotonin receptor modulator is ketanserin and the psychedelic is (R)-methylone, wherein the ketanserin is administered to post-treat at least 120 minutes post to the (R)-methylone. In some embodiments, the serotonin receptor modulator is ketanserin and the psychedelic is (R)-methylone, wherein the ketanserin is administered to post-treat at least 150 minutes post to the (R)-methylone. In some embodiments, the serotonin receptor modulator is ketanserin and the psychedelic is (R)-methylone, wherein the ketanserin is administered to post-treat between about 15 minutes and about 150 minutes post to the (R)-methylone. In some embodiments, the serotonin receptor modulator is ketanserin and the psychedelic is (R)-methylone, wherein the ketanserin is administered to post-treat at least 180 minutes post to the (R)-methylone. In some embodiments, the serotonin receptor modulator is ketanserin and the psychedelic is (R)-methylone, wherein the ketanserin is administered to post-treat at least 210 minutes post to the (R)-methylone. In some embodiments, the serotonin receptor modulator is ketanserin and the psychedelic is (R)-methylone, wherein the ketanserin is administered to post-treat at least 240 minutes post to the (R)-methylone. In some embodiments, the serotonin receptor modulator is ketanserin and the psychedelic is (R)-methylone, wherein the ketanserin is administered to post-treat at least 270 minutes post to the (R)-methylone. In some embodiments, the serotonin receptor modulator is ketanserin and the psychedelic is (R)-methylone, wherein the ketanserin is administered to post-treat at least 300 minutes post to the (R)-methylone. In some embodiments, the serotonin receptor modulator is ketanserin and the psychedelic is (R)-methylone, wherein the ketanserin is administered to post-treat at least 330 minutes post to the (R)-methylone. In some embodiments, the serotonin receptor modulator is ketanserin and the psychedelic is (R)-methylone, wherein the ketanserin is administered to post-treat at least 360 minutes post to the (R)-methylone. In some preferred embodiments, the serotonin receptor modulator is ketanserin and the psychedelic is (R)-methylone, wherein ketanserin is administered to post-treat between about 60 minutes and about 180 minutes post to the administration of (R)-methylone.

In some embodiments, the serotonin receptor modulator is ritanserin and the psychedelic is (R)-methylone, wherein the ritanserin is administered to post-treat at least 15 minutes post to the administration of (R)-methylone. In some embodiments, the serotonin receptor modulator is ritanserin and the psychedelic is (R)-methylone, wherein the ritanserin is administered to post-treat at least 30 minutes post to the (R)-methylone. In some embodiments, the serotonin receptor modulator is ritanserin and the psychedelic is (R)-methylone, wherein the ritanserin is administered to post-treat at least 60 minutes post to the (R)-methylone. In some embodiments, the serotonin receptor modulator is ritanserin and the psychedelic is (R)-methylone, wherein the ritanserin is administered to post-treat between at least 60 minutes and 240 minutes post to the administration or release of (R)-methylone. In some embodiments, the serotonin receptor modulator is ritanserin and the psychedelic is (R)-methylone, wherein the ritanserin is administered to post-treat at least 90 minutes post to the (R)-methylone. In some embodiments, the serotonin receptor modulator is ritanserin and the psychedelic is (R)-methylone, wherein the ritanserin is administered to post-treat at least 120 minutes post to the (R)-methylone. In some embodiments, the serotonin receptor modulator is ritanserin and the psychedelic is (R)-methylone, wherein the ritanserin is administered to post-treat at least 150 minutes post to the (R)-methylone. In some embodiments, the serotonin receptor modulator is ritanserin and the psychedelic is (R)-methylone, wherein the ritanserin is administered to post-treat between about 15 minutes and about 150 minutes post to the (R)-methylone. In some embodiments, the serotonin receptor modulator is ritanserin and the psychedelic is (R)-methylone, wherein the ritanserin is administered to post-treat at least 180 minutes post to the (R)-methylone. In some embodiments, the serotonin receptor modulator is ritanserin and the psychedelic is (R)-methylone, wherein the ritanserin is administered to post-treat at least 210 minutes post to the (R)-methylone. In some embodiments, the serotonin receptor modulator is ritanserin and the psychedelic is (R)-methylone, wherein the ritanserin is administered to post-treat at least 240 minutes post to the (R)-methylone. In some embodiments, the serotonin receptor modulator is ritanserin and the psychedelic is (R)-methylone, wherein the ritanserin is administered to post-treat at least 270 minutes post to the (R)-methylone. In some embodiments, the serotonin receptor modulator is ritanserin and the psychedelic is (R)-methylone, wherein the ritanserin is administered to post-treat at least 300 minutes post to the (R)-methylone. In some embodiments, the serotonin receptor modulator is ritanserin and the psychedelic is (R)-methylone, wherein the ritanserin is administered to post-treat at least 330 minutes post to the (R)-methylone. In some embodiments, the serotonin receptor modulator is ritanserin and the psychedelic is (R)-methylone, wherein the ritanserin is administered to post-treat at least 360 minutes post to the (R)-methylone. In some preferred embodiments, the serotonin receptor modulator is ritanserin and the psychedelic is (R)-methylone, wherein ritanserin is administered to post-treat between about 60 minutes and about 180 minutes post to the administration of (R)-methylone.

In some embodiments, the serotonin receptor modulator is pimavanserin and the psychedelic is (R)-methylone, wherein the pimavanserin is administered to post-treat at least 15 minutes post to the administration of (R)-methylone. In some embodiments, the serotonin receptor modulator is pimavanserin and the psychedelic is (R)-methylone, wherein the pimavanserin is administered to post-treat at least 30 minutes post to the (R)-methylone. In some embodiments, the serotonin receptor modulator is pimavanserin and the psychedelic is (R)-methylone, wherein the pimavanserin is administered to post-treat at least 60 minutes post to the (R)-methylone. In some embodiments, the serotonin receptor modulator is pimavanserin and the psychedelic is (R)-methylone, wherein the pimavanserin is administered to post-treat between at least 60 minutes and 240 minutes post to the administration or release of (R)-methylone. In some embodiments, the serotonin receptor modulator is pimavanserin and the psychedelic is (R)-methylone, wherein the pimavanserin is administered to post-treat at least 90 minutes post to the (R)-methylone. In some embodiments, the serotonin receptor modulator is pimavanserin and the psychedelic is (R)-methylone, wherein the pimavanserin is administered to post-treat at least 120 minutes post to the (R)-methylone. In some embodiments, the serotonin receptor modulator is pimavanserin and the psychedelic is (R)-methylone, wherein the pimavanserin is administered to post-treat at least 150 minutes post to the (R)-methylone. In some embodiments, the serotonin receptor modulator is pimavanserin and the psychedelic is (R)-methylone, wherein the pimavanserin is administered to post-treat between about 15 minutes and about 150 minutes post to the (R)-methylone. In some embodiments, the serotonin receptor modulator is pimavanserin and the psychedelic is (R)-methylone, wherein the pimavanserin is administered to post-treat at least 180 minutes post to the (R)-methylone. In some embodiments, the serotonin receptor modulator is pimavanserin and the psychedelic is (R)-methylone, wherein the pimavanserin is administered to post-treat at least 210 minutes post to the (R)-methylone. In some embodiments, the serotonin receptor modulator is pimavanserin and the psychedelic is (R)-methylone, wherein the pimavanserin is administered to post-treat at least 240 minutes post to the (R)-methylone. In some embodiments, the serotonin receptor modulator is pimavanserin and the psychedelic is (R)-methylone, wherein the pimavanserin is administered to post-treat at least 270 minutes post to the (R)-methylone. In some embodiments, the serotonin receptor modulator is pimavanserin and the psychedelic is (R)-methylone, wherein the pimavanserin is administered to post-treat at least 300 minutes post to the (R)-methylone. In some embodiments, the serotonin receptor modulator is pimavanserin and the psychedelic is (R)-methylone, wherein the pimavanserin is administered to post-treat at least 330 minutes post to the (R)-methylone. In some embodiments, the serotonin receptor modulator is pimavanserin and the psychedelic is (R)-methylone, wherein the pimavanserin is administered to post-treat at least 360 minutes post to the (R)-methylone. In some preferred embodiments, the serotonin receptor modulator is pimavanserin and the psychedelic is (R)-methylone, wherein pimavanserin is administered to post-treat between about 60 minutes and about 180 minutes post to the administration of (R)-methylone.

In some embodiments, the serotonin receptor modulator is nelotanserin and the psychedelic is (R)-methylone, wherein the nelotanserin is administered to post-treat at least 15 minutes post to the administration of (R)-methylone. In some embodiments, the serotonin receptor modulator is nelotanserin and the psychedelic is (R)-methylone, wherein the nelotanserin is administered to post-treat at least 30 minutes post to the (R)-methylone. In some embodiments, the serotonin receptor modulator is nelotanserin and the psychedelic is (R)-methylone, wherein the nelotanserin is administered to post-treat at least 60 minutes post to the (R)-methylone. In some embodiments, the serotonin receptor modulator is nelotanserin and the psychedelic is (R)-methylone, wherein the nelotanserin is administered to post-treat between at least 60 minutes and 240 minutes post to the administration or release of (R)-methylone. In some embodiments, the serotonin receptor modulator is nelotanserin and the psychedelic is (R)-methylone, wherein the nelotanserin is administered to post-treat at least 90 minutes post to the (R)-methylone. In some embodiments, the serotonin receptor modulator is nelotanserin and the psychedelic is (R)-methylone, wherein the nelotanserin is administered to post-treat at least 120 minutes post to the (R)-methylone.

In some embodiments, the serotonin receptor modulator is nelotanserin and the psychedelic is (R)-methylone, wherein the nelotanserin is administered to post-treat at least 150 minutes post to the (R)-methylone. In some embodiments, the serotonin receptor modulator is nelotanserin and the psychedelic is (R)-methylone, wherein the nelotanserin is administered to post-treat between about 15 minutes and about 150 minutes post to the (R)-methylone. In some embodiments, the serotonin receptor modulator is nelotanserin and the psychedelic is (R)-methylone, wherein the nelotanserin is administered to post-treat at least 180 minutes post to the (R)-methylone. In some embodiments, the serotonin receptor modulator is nelotanserin and the psychedelic is (R)-methylone, wherein the nelotanserin is administered to post-treat at least 210 minutes post to the (R)-methylone. In some embodiments, the serotonin receptor modulator is nelotanserin and the psychedelic is (R)-methylone, wherein the nelotanserin is administered to post-treat at least 240 minutes post to the (R)-methylone. In some embodiments, the serotonin receptor modulator is nelotanserin and the psychedelic is (R)-methylone, wherein the nelotanserin is administered to post-treat at least 270 minutes post to the (R)-methylone.

In some embodiments, the serotonin receptor modulator is nelotanserin and the psychedelic is (R)-methylone, wherein the nelotanserin is administered to post-treat at least 300 minutes post to the (R)-methylone. In some embodiments, the serotonin receptor modulator is nelotanserin and the psychedelic is (R)-methylone, wherein the nelotanserin is administered to post-treat at least 330 minutes post to the (R)-methylone. In some embodiments, the serotonin receptor modulator is nelotanserin and the psychedelic is (R)-methylone, wherein the nelotanserin is administered to post-treat at least 360 minutes post to the (R)-methylone. In some preferred embodiments, the serotonin receptor modulator is nelotanserin and the psychedelic is (R)-methylone, wherein nelotanserin is administered to post-treat between about 60 minutes and about 180 minutes post to the administration of (R)-methylone.

In some embodiments, the serotonin receptor modulator is pruvanserin and the psychedelic is (R)-methylone, wherein the pruvanserin is administered to post-treat at least 15 minutes post to the administration of (R)-methylone. In some embodiments, the serotonin receptor modulator is pruvanserin and the psychedelic is (R)-methylone, wherein the pruvanserin is administered to post-treat at least 30 minutes post to the (R)-methylone. In some embodiments, the serotonin receptor modulator is pruvanserin and the psychedelic is (R)-methylone, wherein the pruvanserin is administered to post-treat at least 60 minutes post to the (R)-methylone. In some embodiments, the serotonin receptor modulator is pruvanserin and the psychedelic is (R)-methylone, wherein the pruvanserin is administered to post-treat between at least 60 minutes and 240 minutes post to the administration or release of (R)-methylone. In some embodiments, the serotonin receptor modulator is pruvanserin and the psychedelic is (R)-methylone, wherein the pruvanserin is administered to post-treat at least 90 minutes post to the (R)-methylone. In some embodiments, the serotonin receptor modulator is pruvanserin and the psychedelic is (R)-methylone, wherein the pruvanserin is administered to post-treat at least 120 minutes post to the (R)-methylone. In some embodiments, the serotonin receptor modulator is pruvanserin and the psychedelic is (R)-methylone, wherein the pruvanserin is administered to post-treat at least 150 minutes post to the (R)-methylone. In some embodiments, the serotonin receptor modulator is pruvanserin and the psychedelic is (R)-methylone, wherein the pruvanserin is administered to post-treat between about 15 minutes and about 150 minutes post to the (R)-methylone. In some embodiments, the serotonin receptor modulator is pruvanserin and the psychedelic is (R)-methylone, wherein the pruvanserin is administered to post-treat at least 180 minutes post to the (R)-methylone. In some embodiments, the serotonin receptor modulator is pruvanserin and the psychedelic is (R)-methylone, wherein the pruvanserin is administered to post-treat at least 210 minutes post to the (R)-methylone.

In some embodiments, the serotonin receptor modulator is pruvanserin and the psychedelic is (R)-methylone, wherein the pruvanserin is administered to post-treat at least 240 minutes post to the (R)-methylone. In some embodiments, the serotonin receptor modulator is pruvanserin and the psychedelic is (R)-methylone, wherein the pruvanserin is administered to post-treat at least 270 minutes post to the (R)-methylone. In some embodiments, the serotonin receptor modulator is pruvanserin and the psychedelic is (R)-methylone, wherein the pruvanserin is administered to post-treat at least 300 minutes post to the (R)-methylone. In some embodiments, the serotonin receptor modulator is pruvanserin and the psychedelic is (R)-methylone, wherein the pruvanserin is administered to post-treat at least 330 minutes post to the (R)-methylone. In some embodiments, the serotonin receptor modulator is pruvanserin and the psychedelic is (R)-methylone, wherein the pruvanserin is administered to post-treat at least 360 minutes post to the (R)-methylone. In some preferred embodiments, the serotonin receptor modulator is pruvanserin and the psychedelic is (R)-methylone, wherein pruvanserin is administered to post-treat between about 60 minutes and about 180 minutes post to the administration of (R)-methylone.

In some embodiments, the serotonin receptor modulator is flibanserin and the psychedelic is (R)-methylone, wherein the flibanserin is administered to post-treat at least 15 minutes post to the administration of (R)-methylone. In some embodiments, the serotonin receptor modulator is flibanserin and the psychedelic is (R)-methylone, wherein the flibanserin is administered to post-treat at least 30 minutes post to the (R)-methylone. In some embodiments, the serotonin receptor modulator is flibanserin and the psychedelic is (R)-methylone, wherein the flibanserin is administered to post-treat at least 60 minutes post to the (R)-methylone. In some embodiments, the serotonin receptor modulator is flibanserin and the psychedelic is (R)-methylone, wherein the flibanserin is administered to post-treat at least 90 minutes post to the (R)-methylone. In some embodiments, the serotonin receptor modulator is flibanserin and the psychedelic is (R)-methylone, wherein the flibanserin is administered to post-treat at least 120 minutes post to the (R)-methylone. In some embodiments, the serotonin receptor modulator is flibanserin and the psychedelic is (R)-methylone, wherein the flibanserin is administered to post-treat at least 150 minutes post to the (R)-methylone. In some embodiments, the serotonin receptor modulator is flibanserin and the psychedelic is (R)-methylone, wherein the flibanserin is administered to post-treat between about 15 minutes and about 150 minutes post to the (R)-methylone. In some embodiments, the serotonin receptor modulator is flibanserin and the psychedelic is (R)-methylone, wherein the flibanserin is administered to post-treat at least 180 minutes post to the (R)-methylone. In some embodiments, the serotonin receptor modulator is flibanserin and the psychedelic is (R)-methylone, wherein the flibanserin is administered to post-treat at least 210 minutes post to the (R)-methylone. In some embodiments, the serotonin receptor modulator is flibanserin and the psychedelic is (R)-methylone, wherein the flibanserin is administered to post-treat at least 240 minutes post to the (R)-methylone. In some embodiments, the serotonin receptor modulator is flibanserin and the psychedelic is (R)-methylone, wherein the flibanserin is administered to post-treat at least 270 minutes post to the (R)-methylone. In some embodiments, the serotonin receptor modulator is flibanserin and the psychedelic is (R)-methylone, wherein the flibanserin is administered to post-treat at least 300 minutes post to the (R)-methylone. In some embodiments, the serotonin receptor modulator is flibanserin and the psychedelic is (R)-methylone, wherein the flibanserin is administered to post-treat at least 330 minutes post to the (R)-methylone. In some embodiments, the serotonin receptor modulator is flibanserin and the psychedelic is (R)-methylone, wherein the flibanserin is administered to post-treat at least 360 minutes post to the (R)-methylone. In some preferred embodiments, the serotonin receptor modulator is flibanserin and the psychedelic is (R)-methylone, wherein flibanserin is administered to post-treat between about 60 minutes and about 180 minutes post to the administration of (R)-methylone.

In some embodiments, the serotonin receptor modulator is olanzapine and the psychedelic is (R)-methylone, wherein the olanzapine is administered to post-treat at least 15 minutes post to the administration of (R)-methylone. In some embodiments, the serotonin receptor modulator is olanzapine and the psychedelic is (R)-methylone, wherein the olanzapine is administered to post-treat at least 30 minutes post to the (R)-methylone. In some embodiments, the serotonin receptor modulator is olanzapine and the psychedelic is (R)-methylone, wherein the olanzapine is administered to post-treat at least 60 minutes post to the (R)-methylone. In some embodiments, the serotonin receptor modulator is olanzapine and the psychedelic is (R)-methylone, wherein the olanzapine is administered to post-treat at least 90 minutes post to the (R)-methylone. In some embodiments, the serotonin receptor modulator is olanzapine and the psychedelic is (R)-methylone, wherein the olanzapine is administered to post-treat at least 120 minutes post to the (R)-methylone. In some embodiments, the serotonin receptor modulator is olanzapine and the psychedelic is (R)-methylone, wherein the olanzapine is administered to post-treat at least 150 minutes post to the (R)-methylone. In some embodiments, the serotonin receptor modulator is olanzapine and the psychedelic is (R)-methylone, wherein the olanzapine is administered to post-treat between about 15 minutes and about 150 minutes post to the (R)-methylone. In some embodiments, the serotonin receptor modulator is olanzapine and the psychedelic is (R)-methylone, wherein the olanzapine is administered to post-treat at least 180 minutes post to the (R)-methylone. In some embodiments, the serotonin receptor modulator is olanzapine and the psychedelic is (R)-methylone, wherein the olanzapine is administered to post-treat at least 210 minutes post to the (R)-methylone. In some embodiments, the serotonin receptor modulator is olanzapine and the psychedelic is (R)-methylone, wherein the olanzapine is administered to post-treat at least 240 minutes post to the (R)-methylone. In some embodiments, the serotonin receptor modulator is olanzapine and the psychedelic is (R)-methylone, wherein the olanzapine is administered to post-treat at least 270 minutes post to the (R)-methylone. In some embodiments, the serotonin receptor modulator is olanzapine and the psychedelic is (R)-methylone, wherein the olanzapine is administered to post-treat at least 300 minutes post to the (R)-methylone. In some embodiments, the serotonin receptor modulator is olanzapine and the psychedelic is (R)-methylone, wherein the olanzapine is administered to post-treat at least 330 minutes post to the (R)-methylone. In some embodiments, the serotonin receptor modulator is olanzapine and the psychedelic is (R)-methylone, wherein the olanzapine is administered to post-treat at least 360 minutes post to the (R)-methylone. In some preferred embodiments, the serotonin receptor modulator is olanzapine and the psychedelic is (R)-methylone, wherein olanzapine is administered to post-treat between about 60 minutes and about 180 minutes post to the administration of (R)-methylone.

In some embodiments, the serotonin receptor modulator is quetiapine and the psychedelic is (R)-methylone, wherein the quetiapine is administered to post-treat at least 15 minutes post to the administration of (R)-methylone. In some embodiments, the serotonin receptor modulator is quetiapine and the psychedelic is (R)-methylone, wherein the quetiapine is administered to post-treat at least 30 minutes post to the (R)-methylone. In some embodiments, the serotonin receptor modulator is quetiapine and the psychedelic is (R)-methylone, wherein the quetiapine is administered to post-treat at least 60 minutes post to the (R)-methylone. In some embodiments, the serotonin receptor modulator is quetiapine and the psychedelic is (R)-methylone, wherein the quetiapine is administered to post-treat at least 90 minutes post to the (R)-methylone. In some embodiments, the serotonin receptor modulator is quetiapine and the psychedelic is (R)-methylone, wherein the quetiapine is administered to post-treat at least 120 minutes post to the (R)-methylone. In some embodiments, the serotonin receptor modulator is quetiapine and the psychedelic is (R)-methylone, wherein the quetiapine is administered to post-treat at least 150 minutes post to the (R)-methylone. In some embodiments, the serotonin receptor modulator is quetiapine and the psychedelic is (R)-methylone, wherein the quetiapine is administered to post-treat between about 15 minutes and about 150 minutes post to the (R)-methylone. In some embodiments, the serotonin receptor modulator is quetiapine and the psychedelic is (R)-methylone, wherein the quetiapine is administered to post-treat at least 180 minutes post to the (R)-methylone. In some embodiments, the serotonin receptor modulator is quetiapine and the psychedelic is (R)-methylone, wherein the quetiapine is administered to post-treat at least 210 minutes post to the (R)-methylone. In some embodiments, the serotonin receptor modulator is quetiapine and the psychedelic is (R)-methylone, wherein the quetiapine is administered to post-treat at least 240 minutes post to the (R)-methylone. In some embodiments, the serotonin receptor modulator is quetiapine and the psychedelic is (R)-methylone, wherein the quetiapine is administered to post-treat at least 270 minutes post to the (R)-methylone. In some embodiments, the serotonin receptor modulator is quetiapine and the psychedelic is (R)-methylone, wherein the quetiapine is administered to post-treat at least 300 minutes post to the (R)-methylone. In some embodiments, the serotonin receptor modulator is quetiapine and the psychedelic is (R)-methylone, wherein the quetiapine is administered to post-treat at least 330 minutes post to the (R)-methylone. In some embodiments, the serotonin receptor modulator is quetiapine and the psychedelic is (R)-methylone, wherein the quetiapine is administered to post-treat at least 360 minutes post to the (R)-methylone. In some preferred embodiments, the serotonin receptor modulator is quetiapine and the psychedelic is (R)-methylone, wherein quetiapine is administered to post-treat between about 60 minutes and about 180 minutes post to the administration of (R)-methylone.

In some embodiments, the serotonin receptor modulator is risperidone and the psychedelic is (R)-methylone, wherein the risperidone is administered to post-treat at least 15 minutes post to the administration of (R)-methylone. In some embodiments, the serotonin receptor modulator is risperidone and the psychedelic is (R)-methylone, wherein the risperidone is administered to post-treat at least 30 minutes post to the (R)-methylone. In some embodiments, the serotonin receptor modulator is risperidone and the psychedelic is (R)-methylone, wherein the risperidone is administered to post-treat at least 60 minutes post to the (R)-methylone. In some embodiments, the serotonin receptor modulator is risperidone and the psychedelic is (R)-methylone, wherein the risperidone is administered to post-treat at least 90 minutes post to the (R)-methylone. In some embodiments, the serotonin receptor modulator is risperidone and the psychedelic is (R)-methylone, wherein the risperidone is administered to post-treat at least 120 minutes post to the (R)-methylone. In some embodiments, the serotonin receptor modulator is risperidone and the psychedelic is (R)-methylone, wherein the risperidone is administered to post-treat at least 150 minutes post to the (R)-methylone. In some embodiments, the serotonin receptor modulator is risperidone and the psychedelic is (R)-methylone, wherein the risperidone is administered to post-treat between about 15 minutes and about 150 minutes post to the (R)-methylone. In some embodiments, the serotonin receptor modulator is risperidone and the psychedelic is (R)-methylone, wherein the risperidone is administered to post-treat at least 180 minutes post to the (R)-methylone. In some embodiments, the serotonin receptor modulator is risperidone and the psychedelic is (R)-methylone, wherein the risperidone is administered to post-treat at least 210 minutes post to the (R)-methylone. In some embodiments, the serotonin receptor modulator is risperidone and the psychedelic is (R)-methylone, wherein the risperidone is administered to post-treat at least 240 minutes post to the (R)-methylone. In some embodiments, the serotonin receptor modulator is risperidone and the psychedelic is (R)-methylone, wherein the risperidone is administered to post-treat at least 270 minutes post to the (R)-methylone. In some embodiments, the serotonin receptor modulator is risperidone and the psychedelic is (R)-methylone, wherein the risperidone is administered to post-treat at least 300 minutes post to the (R)-methylone. In some embodiments, the serotonin receptor modulator is risperidone and the psychedelic is (R)-methylone, wherein the risperidone is administered to post-treat at least 330 minutes post to the (R)-methylone. In some embodiments, the serotonin receptor modulator is risperidone and the psychedelic is (R)-methylone, wherein the risperidone is administered to post-treat at least 360 minutes post to the (R)-methylone. In some preferred embodiments, the serotonin receptor modulator is risperidone and the psychedelic is (R)-methylone, wherein risperidone is administered to post-treat between about 60 minutes and about 180 minutes post to the administration of (R)-methylone.

In some embodiments, the serotonin receptor modulator is eplivanserin and the psychedelic is (S)-methylone, wherein the eplivanserin is administered to post-treat at least 15 minutes post to the administration of (S)-methylone. In some embodiments, the serotonin receptor modulator is eplivanserin and the psychedelic is (S)-methylone, wherein the eplivanserin is administered to post-treat at least 30 minutes post to the (S)-methylone. In some embodiments, the serotonin receptor modulator is eplivanserin and the psychedelic is (S)-methylone, wherein the eplivanserin is administered to post-treat at least 60 minutes post to the (S)-methylone. In some embodiments, the serotonin receptor modulator is eplivanserin and the psychedelic is (S)-methylone, wherein the eplivanserin is administered to post-treat at least 90 minutes post to the (S)-methylone. In some embodiments, the serotonin receptor modulator is eplivanserin and the psychedelic is (S)-methylone, wherein the eplivanserin is administered to post-treat between at least 30 minutes post and 360 minutes post to the release or administration of the (S)-methylone. In some embodiments, the serotonin receptor modulator is eplivanserin and the psychedelic is (S)-methylone, wherein the eplivanserin is administered to post-treat between at least 60 minutes post and 360 minutes post to the release or administration the (S)-methylone. In some embodiments, the serotonin receptor modulator is eplivanserin and the psychedelic is (S)-methylone, wherein the eplivanserin is administered to post-treat between at least 90 minutes and 240 minutes post to the (S)-methylone. In some embodiments, the serotonin receptor modulator is eplivanserin and the psychedelic is (S)-methylone, wherein the eplivanserin is administered to post-treat at least 120 minutes post to the (S)-methylone. In some embodiments, the serotonin receptor modulator is eplivanserin and the psychedelic is (S)-methylone, wherein the eplivanserin is administered to post-treat at least 150 minutes post to the (S)-methylone. In some embodiments, the serotonin receptor modulator is eplivanserin and the psychedelic is (S)-methylone, wherein the eplivanserin is administered to post-treat between about 15 minutes and about 150 minutes post to the (S)-methylone. In some embodiments, the serotonin receptor modulator is eplivanserin and the psychedelic is (S)-methylone, wherein the eplivanserin is administered to post-treat at least 180 minutes post to the (S)-methylone. In some embodiments, the serotonin receptor modulator is eplivanserin and the psychedelic is (S)-methylone, wherein the eplivanserin is administered to post-treat at least 210 minutes post to the (S)-methylone. In some embodiments, the serotonin receptor modulator is eplivanserin and the psychedelic is (S)-methylone, wherein the eplivanserin is administered to post-treat at least 240 minutes post to the (S)-methylone.

In some embodiments, the serotonin receptor modulator is eplivanserin and the psychedelic is (S)-methylone, wherein the eplivanserin is administered to post-treat at least 270 minutes post to the (S)-methylone. In some embodiments, the serotonin receptor modulator is eplivanserin and the psychedelic is (S)-methylone, wherein the eplivanserin is administered to post-treat at least 300 minutes post to the (S)-methylone. In some embodiments, the serotonin receptor modulator is eplivanserin and the psychedelic is (S)-methylone, wherein the eplivanserin is administered to post-treat at least 330 minutes post to the (S)-methylone. In some embodiments, the serotonin receptor modulator is eplivanserin and the psychedelic is (S)-methylone, wherein the eplivanserin is administered to post-treat at least 360 minutes post to the (S)-methylone.

In some preferred embodiments, the serotonin receptor modulator is eplivanserin and the psychedelic is (S)-methylone, wherein eplivanserin is administered to post-treat between about 60 minutes and about 180 minutes post to the administration of (S)-methylone.

In some embodiments, the serotonin receptor modulator is volinanserin and the psychedelic is (S)-methylone, wherein the volinanserin is administered to post-treat at least 15 minutes post to the administration of (S)-methylone. In some embodiments, the serotonin receptor modulator is volinanserin and the psychedelic is (S)-methylone, wherein the volinanserin is administered to post-treat at least 30 minutes post to the (S)-methylone. In some embodiments, the serotonin receptor modulator is volinanserin and the psychedelic is (S)-methylone, wherein the volinanserin is administered to post-treat at least 60 minutes post to the (S)-methylone. In some embodiments, the serotonin receptor modulator is volinanserin and the psychedelic is (S)-methylone, wherein the volinanserin is administered to post-treat a subject between at least 15 minutes and 360 minutes post to the administration or release of (S)-methylone. In some embodiments, the serotonin receptor modulator is volinanserin and the psychedelic is (S)-methylone, wherein the volinanserin is administered to post-treat between at least 30 minutes and 360 minutes post to the administration or release of (S)-methylone. In some embodiments, the serotonin receptor modulator is volinanserin and the psychedelic is (S)-methylone, wherein the volinanserin is administered to post-treat between at least 60 minutes and 240 minutes post to the administration or release of (S)-methylone. In some embodiments, the serotonin receptor modulator is volinanserin and the psychedelic is (S)-methylone, wherein the volinanserin is administered to post-treat at least 90 minutes post to the (S)-methylone. In some embodiments, the serotonin receptor modulator is volinanserin and the psychedelic is (S)-methylone, wherein the volinanserin is administered to post-treat at least 120 minutes post to the (S)-methylone.

In some embodiments, the serotonin receptor modulator is volinanserin and the psychedelic is (S)-methylone, wherein the volinanserin is administered to post-treat at least 150 minutes post to the (S)-methylone. In some embodiments, the serotonin receptor modulator is volinanserin and the psychedelic is (S)-methylone, wherein the volinanserin is administered to post-treat between about 15 minutes and about 150 minutes post to the (S)-methylone. In some embodiments, the serotonin receptor modulator is volinanserin and the psychedelic is (S)-methylone, wherein the volinanserin is administered to post-treat at least 180 minutes post to the (S)-methylone. In some embodiments, the serotonin receptor modulator is volinanserin and the psychedelic is (S)-methylone, wherein the volinanserin is administered to post-treat at least 210 minutes post to the (S)-methylone. In some embodiments, the serotonin receptor modulator is volinanserin and the psychedelic is (S)-methylone, wherein the volinanserin is administered to post-treat at least 240 minutes post to the (S)-methylone. In some embodiments, the serotonin receptor modulator is volinanserin and the psychedelic is (S)-methylone, wherein the volinanserin is administered to post-treat at least 270 minutes post to the (S)-methylone. In some embodiments, the serotonin receptor modulator is volinanserin and the psychedelic is (S)-methylone, wherein the volinanserin is administered to post-treat at least 300 minutes post to the (S)-methylone. In some embodiments, the serotonin receptor modulator is volinanserin and the psychedelic is (S)-methylone, wherein the volinanserin is administered to post-treat at least 330 minutes post to the (S)-methylone. In some embodiments, the serotonin receptor modulator is volinanserin and the psychedelic is (S)-methylone, wherein the volinanserin is administered to post-treat at least 360 minutes post to the (S)-methylone. In some preferred embodiments, the serotonin receptor modulator is volinanserin and the psychedelic is (S)-methylone, wherein volinanserin is administered to post-treat between about 60 minutes and about 180 minutes post to the administration of (S)-methylone.

In some embodiments, the serotonin receptor modulator is ketanserin and the psychedelic is (S)-methylone, wherein the ketanserin is administered to post-treat at least 15 minutes post to the administration of (S)-methylone. In some embodiments, the serotonin receptor modulator is ketanserin and the psychedelic is (S)-methylone, wherein the ketanserin is administered to post-treat at least 30 minutes post to the (S)-methylone. In some embodiments, the serotonin receptor modulator is ketanserin and the psychedelic is (S)-methylone, wherein the ketanserin is administered to post-treat at least 60 minutes post to the (S)-methylone. In some embodiments, the serotonin receptor modulator is ketanserin and the psychedelic is (S)-methylone, wherein the ketanserin is administered to post-treat between at least 30 minutes and 360 minutes post to the administration or release (S)-methylone. In some embodiments, the serotonin receptor modulator is ketanserin and the psychedelic is (S)-methylone, wherein the ketanserin is administered to post-treat between at least 60 minutes and 240 minutes post to the administration or release of (S)-methylone. In some embodiments, the serotonin receptor modulator is ketanserin and the psychedelic is (S)-methylone, wherein the ketanserin is administered to post-treat at least 90 minutes post to the (S)-methylone. In some embodiments, the serotonin receptor modulator is ketanserin and the psychedelic is (S)-methylone, wherein the ketanserin is administered to post-treat at least 120 minutes post to the (S)-methylone. In some embodiments, the serotonin receptor modulator is ketanserin and the psychedelic is (S)-methylone, wherein the ketanserin is administered to post-treat at least 150 minutes post to the (S)-methylone. In some embodiments, the serotonin receptor modulator is ketanserin and the psychedelic is (S)-methylone, wherein the ketanserin is administered to post-treat between about 15 minutes and about 150 minutes post to the (S)-methylone. In some embodiments, the serotonin receptor modulator is ketanserin and the psychedelic is (S)-methylone, wherein the ketanserin is administered to post-treat at least 180 minutes post to the (S)-methylone. In some embodiments, the serotonin receptor modulator is ketanserin and the psychedelic is (S)-methylone, wherein the ketanserin is administered to post-treat at least 210 minutes post to the (S)-methylone. In some embodiments, the serotonin receptor modulator is ketanserin and the psychedelic is (S)-methylone, wherein the ketanserin is administered to post-treat at least 240 minutes post to the (S)-methylone. In some embodiments, the serotonin receptor modulator is ketanserin and the psychedelic is (S)-methylone, wherein the ketanserin is administered to post-treat at least 270 minutes post to the (S)-methylone. In some embodiments, the serotonin receptor modulator is ketanserin and the psychedelic is (S)-methylone, wherein the ketanserin is administered to post-treat at least 300 minutes post to the (S)-methylone. In some embodiments, the serotonin receptor modulator is ketanserin and the psychedelic is (S)-methylone, wherein the ketanserin is administered to post-treat at least 330 minutes post to the (S)-methylone. In some embodiments, the serotonin receptor modulator is ketanserin and the psychedelic is (S)-methylone, wherein the ketanserin is administered to post-treat at least 360 minutes post to the (S)-methylone. In some preferred embodiments, the serotonin receptor modulator is ketanserin and the psychedelic is (S)-methylone, wherein ketanserin is administered to post-treat between about 60 minutes and about 180 minutes post to the administration of (S)-methylone.

In some embodiments, the serotonin receptor modulator is ritanserin and the psychedelic is (S)-methylone, wherein the ritanserin is administered to post-treat at least 15 minutes post to the administration of (S)-methylone. In some embodiments, the serotonin receptor modulator is ritanserin and the psychedelic is (S)-methylone, wherein the ritanserin is administered to post-treat at least 30 minutes post to the (S)-methylone. In some embodiments, the serotonin receptor modulator is ritanserin and the psychedelic is (S)-methylone, wherein the ritanserin is administered to post-treat at least 60 minutes post to the (S)-methylone. In some embodiments, the serotonin receptor modulator is ritanserin and the psychedelic is (S)-methylone, wherein the ritanserin is administered to post-treat between at least 60 minutes and 240 minutes post to the administration or release of (S)-methylone. In some embodiments, the serotonin receptor modulator is ritanserin and the psychedelic is (S)-methylone, wherein the ritanserin is administered to post-treat at least 90 minutes post to the (S)-methylone. In some embodiments, the serotonin receptor modulator is ritanserin and the psychedelic is (S)-methylone, wherein the ritanserin is administered to post-treat at least 120 minutes post to the (S)-methylone. In some embodiments, the serotonin receptor modulator is ritanserin and the psychedelic is (S)-methylone, wherein the ritanserin is administered to post-treat at least 150 minutes post to the (S)-methylone. In some embodiments, the serotonin receptor modulator is ritanserin and the psychedelic is (S)-methylone, wherein the ritanserin is administered to post-treat between about 15 minutes and about 150 minutes post to the (S)-methylone. In some embodiments, the serotonin receptor modulator is ritanserin and the psychedelic is (S)-methylone, wherein the ritanserin is administered to post-treat at least 180 minutes post to the (S)-methylone. In some embodiments, the serotonin receptor modulator is ritanserin and the psychedelic is (S)-methylone, wherein the ritanserin is administered to post-treat at least 210 minutes post to the (S)-methylone. In some embodiments, the serotonin receptor modulator is ritanserin and the psychedelic is (S)-methylone, wherein the ritanserin is administered to post-treat at least 240 minutes post to the (S)-methylone. In some embodiments, the serotonin receptor modulator is ritanserin and the psychedelic is (S)-methylone, wherein the ritanserin is administered to post-treat at least 270 minutes post to the (S)-methylone. In some embodiments, the serotonin receptor modulator is ritanserin and the psychedelic is (S)-methylone, wherein the ritanserin is administered to post-treat at least 300 minutes post to the (S)-methylone. In some embodiments, the serotonin receptor modulator is ritanserin and the psychedelic is (S)-methylone, wherein the ritanserin is administered to post-treat at least 330 minutes post to the (S)-methylone. In some embodiments, the serotonin receptor modulator is ritanserin and the psychedelic is (S)-methylone, wherein the ritanserin is administered to post-treat at least 360 minutes post to the (S)-methylone. In some preferred embodiments, the serotonin receptor modulator is ritanserin and the psychedelic is (S)-methylone, wherein ritanserin is administered to post-treat between about 60 minutes and about 180 minutes post to the administration of (S)-methylone.

In some embodiments, the serotonin receptor modulator is pimavanserin and the psychedelic is (S)-methylone, wherein the pimavanserin is administered to post-treat at least 15 minutes post to the administration of (S)-methylone. In some embodiments, the serotonin receptor modulator is pimavanserin and the psychedelic is (S)-methylone, wherein the pimavanserin is administered to post-treat at least 30 minutes post to the (S)-methylone. In some embodiments, the serotonin receptor modulator is pimavanserin and the psychedelic is (S)-methylone, wherein the pimavanserin is administered to post-treat at least 60 minutes post to the (S)-methylone. In some embodiments, the serotonin receptor modulator is pimavanserin and the psychedelic is (S)-methylone, wherein the pimavanserin is administered to post-treat between at least 60 minutes and 240 minutes post to the administration or release of (S)-methylone. In some embodiments, the serotonin receptor modulator is pimavanserin and the psychedelic is (S)-methylone, wherein the pimavanserin is administered to post-treat at least 90 minutes post to the (S)-methylone. In some embodiments, the serotonin receptor modulator is pimavanserin and the psychedelic is (S)-methylone, wherein the pimavanserin is administered to post-treat at least 120 minutes post to the (S)-methylone. In some embodiments, the serotonin receptor modulator is pimavanserin and the psychedelic is (S)-methylone, wherein the pimavanserin is administered to post-treat at least 150 minutes post to the (S)-methylone. In some embodiments, the serotonin receptor modulator is pimavanserin and the psychedelic is (S)-methylone, wherein the pimavanserin is administered to post-treat between about 15 minutes and about 150 minutes post to the (S)-methylone. In some embodiments, the serotonin receptor modulator is pimavanserin and the psychedelic is (S)-methylone, wherein the pimavanserin is administered to post-treat at least 180 minutes post to the (S)-methylone. In some embodiments, the serotonin receptor modulator is pimavanserin and the psychedelic is (S)-methylone, wherein the pimavanserin is administered to post-treat at least 210 minutes post to the (S)-methylone. In some embodiments, the serotonin receptor modulator is pimavanserin and the psychedelic is (S)-methylone, wherein the pimavanserin is administered to post-treat at least 240 minutes post to the (S)-methylone. In some embodiments, the serotonin receptor modulator is pimavanserin and the psychedelic is (S)-methylone, wherein the pimavanserin is administered to post-treat at least 270 minutes post to the (S)-methylone. In some embodiments, the serotonin receptor modulator is pimavanserin and the psychedelic is (S)-methylone, wherein the pimavanserin is administered to post-treat at least 300 minutes post to the (S)-methylone. In some embodiments, the serotonin receptor modulator is pimavanserin and the psychedelic is (S)-methylone, wherein the pimavanserin is administered to post-treat at least 330 minutes post to the (S)-methylone. In some embodiments, the serotonin receptor modulator is pimavanserin and the psychedelic is (S)-methylone, wherein the pimavanserin is administered to post-treat at least 360 minutes post to the (S)-methylone. In some preferred embodiments, the serotonin receptor modulator is pimavanserin and the psychedelic is (S)-methylone, wherein pimavanserin is administered to post-treat between about 60 minutes and about 180 minutes post to the administration of (S)-methylone.

In some embodiments, the serotonin receptor modulator is nelotanserin and the psychedelic is (S)-methylone, wherein the nelotanserin is administered to post-treat at least 15 minutes post to the administration of (S)-methylone. In some embodiments, the serotonin receptor modulator is nelotanserin and the psychedelic is (S)-methylone, wherein the nelotanserin is administered to post-treat at least 30 minutes post to the (S)-methylone. In some embodiments, the serotonin receptor modulator is nelotanserin and the psychedelic is (S)-methylone, wherein the nelotanserin is administered to post-treat at least 60 minutes post to the (S)-methylone. In some embodiments, the serotonin receptor modulator is nelotanserin and the psychedelic is (S)-methylone, wherein the nelotanserin is administered to post-treat between at least 60 minutes and 240 minutes post to the administration or release of (S)-methylone. In some embodiments, the serotonin receptor modulator is nelotanserin and the psychedelic is (S)-methylone, wherein the nelotanserin is administered to post-treat at least 90 minutes post to the (S)-methylone. In some embodiments, the serotonin receptor modulator is nelotanserin and the psychedelic is (S)-methylone, wherein the nelotanserin is administered to post-treat at least 120 minutes post to the (S)-methylone.

In some embodiments, the serotonin receptor modulator is nelotanserin and the psychedelic is (S)-methylone, wherein the nelotanserin is administered to post-treat at least 150 minutes post to the (S)-methylone. In some embodiments, the serotonin receptor modulator is nelotanserin and the psychedelic is (S)-methylone, wherein the nelotanserin is administered to post-treat between about 15 minutes and about 150 minutes post to the (S)-methylone. In some embodiments, the serotonin receptor modulator is nelotanserin and the psychedelic is (S)-methylone, wherein the nelotanserin is administered to post-treat at least 180 minutes post to the (S)-methylone. In some embodiments, the serotonin receptor modulator is nelotanserin and the psychedelic is (S)-methylone, wherein the nelotanserin is administered to post-treat at least 210 minutes post to the (S)-methylone. In some embodiments, the serotonin receptor modulator is nelotanserin and the psychedelic is (S)-methylone, wherein the nelotanserin is administered to post-treat at least 240 minutes post to the (S)-methylone. In some embodiments, the serotonin receptor modulator is nelotanserin and the psychedelic is (S)-methylone, wherein the nelotanserin is administered to post-treat at least 270 minutes post to the (S)-methylone.

In some embodiments, the serotonin receptor modulator is nelotanserin and the psychedelic is (S)-methylone, wherein the nelotanserin is administered to post-treat at least 300 minutes post to the (S)-methylone. In some embodiments, the serotonin receptor modulator is nelotanserin and the psychedelic is (S)-methylone, wherein the nelotanserin is administered to post-treat at least 330 minutes post to the (S)-methylone. In some embodiments, the serotonin receptor modulator is nelotanserin and the psychedelic is (S)-methylone, wherein the nelotanserin is administered to post-treat at least 360 minutes post to the (S)-methylone. In some preferred embodiments, the serotonin receptor modulator is nelotanserin and the psychedelic is (S)-methylone, wherein nelotanserin is administered to post-treat between about 60 minutes and about 180 minutes post to the administration of (S)-methylone.

In some embodiments, the serotonin receptor modulator is pruvanserin and the psychedelic is (S)-methylone, wherein the pruvanserin is administered to post-treat at least 15 minutes post to the administration of (S)-methylone. In some embodiments, the serotonin receptor modulator is pruvanserin and the psychedelic is (S)-methylone, wherein the pruvanserin is administered to post-treat at least 30 minutes post to the (S)-methylone. In some embodiments, the serotonin receptor modulator is pruvanserin and the psychedelic is (S)-methylone, wherein the pruvanserin is administered to post-treat at least 60 minutes post to the (S)-methylone. In some embodiments, the serotonin receptor modulator is pruvanserin and the psychedelic is (S)-methylone, wherein the pruvanserin is administered to post-treat between at least 60 minutes and 240 minutes post to the administration or release of (S)-methylone. In some embodiments, the serotonin receptor modulator is pruvanserin and the psychedelic is (S)-methylone, wherein the pruvanserin is administered to post-treat at least 90 minutes post to the (S)-methylone. In some embodiments, the serotonin receptor modulator is pruvanserin and the psychedelic is (S)-methylone, wherein the pruvanserin is administered to post-treat at least 120 minutes post to the (S)-methylone. In some embodiments, the serotonin receptor modulator is pruvanserin and the psychedelic is (S)-methylone, wherein the pruvanserin is administered to post-treat at least 150 minutes post to the (S)-methylone. In some embodiments, the serotonin receptor modulator is pruvanserin and the psychedelic is (S)-methylone, wherein the pruvanserin is administered to post-treat between about 15 minutes and about 150 minutes post to the (S)-methylone. In some embodiments, the serotonin receptor modulator is pruvanserin and the psychedelic is (S)-methylone, wherein the pruvanserin is administered to post-treat at least 180 minutes post to the (S)-methylone. In some embodiments, the serotonin receptor modulator is pruvanserin and the psychedelic is (S)-methylone, wherein the pruvanserin is administered to post-treat at least 210 minutes post to the (S)-methylone.

In some embodiments, the serotonin receptor modulator is pruvanserin and the psychedelic is (S)-methylone, wherein the pruvanserin is administered to post-treat at least 240 minutes post to the (S)-methylone. In some embodiments, the serotonin receptor modulator is pruvanserin and the psychedelic is (S)-methylone, wherein the pruvanserin is administered to post-treat at least 270 minutes post to the (S)-methylone. In some embodiments, the serotonin receptor modulator is pruvanserin and the psychedelic is (S)-methylone, wherein the pruvanserin is administered to post-treat at least 300 minutes post to the (S)-methylone. In some embodiments, the serotonin receptor modulator is pruvanserin and the psychedelic is (S)-methylone, wherein the pruvanserin is administered to post-treat at least 330 minutes post to the (S)-methylone. In some embodiments, the serotonin receptor modulator is pruvanserin and the psychedelic is (S)-methylone, wherein the pruvanserin is administered to post-treat at least 360 minutes post to the (S)-methylone. In some preferred embodiments, the serotonin receptor modulator is pruvanserin and the psychedelic is (S)-methylone, wherein pruvanserin is administered to post-treat between about 60 minutes and about 180 minutes post to the administration of (S)-methylone.

In some embodiments, the serotonin receptor modulator is flibanserin and the psychedelic is (S)-methylone, wherein the flibanserin is administered to post-treat at least 15 minutes post to the administration of (S)-methylone. In some embodiments, the serotonin receptor modulator is flibanserin and the psychedelic is (S)-methylone, wherein the flibanserin is administered to post-treat at least 30 minutes post to the (S)-methylone. In some embodiments, the serotonin receptor modulator is flibanserin and the psychedelic is (S)-methylone, wherein the flibanserin is administered to post-treat at least 60 minutes post to the (S)-methylone. In some embodiments, the serotonin receptor modulator is flibanserin and the psychedelic is (S)-methylone, wherein the flibanserin is administered to post-treat at least 90 minutes post to the (S)-methylone. In some embodiments, the serotonin receptor modulator is flibanserin and the psychedelic is (S)-methylone, wherein the flibanserin is administered to post-treat at least 120 minutes post to the (S)-methylone. In some embodiments, the serotonin receptor modulator is flibanserin and the psychedelic is (S)-methylone, wherein the flibanserin is administered to post-treat at least 150 minutes post to the (S)-methylone. In some embodiments, the serotonin receptor modulator is flibanserin and the psychedelic is (S)-methylone, wherein the flibanserin is administered to post-treat between about 15 minutes and about 150 minutes post to the (S)-methylone. In some embodiments, the serotonin receptor modulator is flibanserin and the psychedelic is (S)-methylone, wherein the flibanserin is administered to post-treat at least 180 minutes post to the (S)-methylone. In some embodiments, the serotonin receptor modulator is flibanserin and the psychedelic is (S)-methylone, wherein the flibanserin is administered to post-treat at least 210 minutes post to the (S)-methylone. In some embodiments, the serotonin receptor modulator is flibanserin and the psychedelic is (S)-methylone, wherein the flibanserin is administered to post-treat at least 240 minutes post to the (S)-methylone. In some embodiments, the serotonin receptor modulator is flibanserin and the psychedelic is (S)-methylone, wherein the flibanserin is administered to post-treat at least 270 minutes post to the (S)-methylone. In some embodiments, the serotonin receptor modulator is flibanserin and the psychedelic is (S)-methylone, wherein the flibanserin is administered to post-treat at least 300 minutes post to the (S)-methylone. In some embodiments, the serotonin receptor modulator is flibanserin and the psychedelic is (S)-methylone, wherein the flibanserin is administered to post-treat at least 330 minutes post to the (S)-methylone. In some embodiments, the serotonin receptor modulator is flibanserin and the psychedelic is (S)-methylone, wherein the flibanserin is administered to post-treat at least 360 minutes post to the (S)-methylone. In some preferred embodiments, the serotonin receptor modulator is flibanserin and the psychedelic is (S)-methylone, wherein flibanserin is administered to post-treat between about 60 minutes and about 180 minutes post to the administration of (S)-methylone.

In some embodiments, the serotonin receptor modulator is olanzapine and the psychedelic is (S)-methylone, wherein the olanzapine is administered to post-treat at least 15 minutes post to the administration of (S)-methylone. In some embodiments, the serotonin receptor modulator is olanzapine and the psychedelic is (S)-methylone, wherein the olanzapine is administered to post-treat at least 30 minutes post to the (S)-methylone. In some embodiments, the serotonin receptor modulator is olanzapine and the psychedelic is (S)-methylone, wherein the olanzapine is administered to post-treat at least 60 minutes post to the (S)-methylone. In some embodiments, the serotonin receptor modulator is olanzapine and the psychedelic is (S)-methylone, wherein the olanzapine is administered to post-treat at least 90 minutes post to the (S)-methylone. In some embodiments, the serotonin receptor modulator is olanzapine and the psychedelic is (S)-methylone, wherein the olanzapine is administered to post-treat at least 120 minutes post to the (S)-methylone. In some embodiments, the serotonin receptor modulator is olanzapine and the psychedelic is (S)-methylone, wherein the olanzapine is administered to post-treat at least 150 minutes post to the (S)-methylone. In some embodiments, the serotonin receptor modulator is olanzapine and the psychedelic is (S)-methylone, wherein the olanzapine is administered to post-treat between about 15 minutes and about 150 minutes post to the (S)-methylone. In some embodiments, the serotonin receptor modulator is olanzapine and the psychedelic is (S)-methylone, wherein the olanzapine is administered to post-treat at least 180 minutes post to the (S)-methylone. In some embodiments, the serotonin receptor modulator is olanzapine and the psychedelic is (S)-methylone, wherein the olanzapine is administered to post-treat at least 210 minutes post to the (S)-methylone. In some embodiments, the serotonin receptor modulator is olanzapine and the psychedelic is (S)-methylone, wherein the olanzapine is administered to post-treat at least 240 minutes post to the (S)-methylone. In some embodiments, the serotonin receptor modulator is olanzapine and the psychedelic is (S)-methylone, wherein the olanzapine is administered to post-treat at least 270 minutes post to the (S)-methylone. In some embodiments, the serotonin receptor modulator is olanzapine and the psychedelic is (S)-methylone, wherein the olanzapine is administered to post-treat at least 300 minutes post to the (S)-methylone. In some embodiments, the serotonin receptor modulator is olanzapine and the psychedelic is (S)-methylone, wherein the olanzapine is administered to post-treat at least 330 minutes post to the (S)-methylone. In some embodiments, the serotonin receptor modulator is olanzapine and the psychedelic is (S)-methylone, wherein the olanzapine is administered to post-treat at least 360 minutes post to the (S)-methylone. In some preferred embodiments, the serotonin receptor modulator is olanzapine and the psychedelic is (S)-methylone, wherein olanzapine is administered to post-treat between about 60 minutes and about 180 minutes post to the administration of (S)-methylone.

In some embodiments, the serotonin receptor modulator is quetiapine and the psychedelic is (S)-methylone, wherein the quetiapine is administered to post-treat at least 15 minutes post to the administration of (S)-methylone. In some embodiments, the serotonin receptor modulator is quetiapine and the psychedelic is (S)-methylone, wherein the quetiapine is administered to post-treat at least 30 minutes post to the (S)-methylone. In some embodiments, the serotonin receptor modulator is quetiapine and the psychedelic is (S)-methylone, wherein the quetiapine is administered to post-treat at least 60 minutes post to the (S)-methylone. In some embodiments, the serotonin receptor modulator is quetiapine and the psychedelic is (S)-methylone, wherein the quetiapine is administered to post-treat at least 90 minutes post to the (S)-methylone. In some embodiments, the serotonin receptor modulator is quetiapine and the psychedelic is (S)-methylone, wherein the quetiapine is administered to post-treat at least 120 minutes post to the (S)-methylone. In some embodiments, the serotonin receptor modulator is quetiapine and the psychedelic is (S)-methylone, wherein the quetiapine is administered to post-treat at least 150 minutes post to the (S)-methylone. In some embodiments, the serotonin receptor modulator is quetiapine and the psychedelic is (S)-methylone, wherein the quetiapine is administered to post-treat between about 15 minutes and about 150 minutes post to the (S)-methylone. In some embodiments, the serotonin receptor modulator is quetiapine and the psychedelic is (S)-methylone, wherein the quetiapine is administered to post-treat at least 180 minutes post to the (S)-methylone. In some embodiments, the serotonin receptor modulator is quetiapine and the psychedelic is (S)-methylone, wherein the quetiapine is administered to post-treat at least 210 minutes post to the (S)-methylone. In some embodiments, the serotonin receptor modulator is quetiapine and the psychedelic is (S)-methylone, wherein the quetiapine is administered to post-treat at least 240 minutes post to the (S)-methylone. In some embodiments, the serotonin receptor modulator is quetiapine and the psychedelic is (S)-methylone, wherein the quetiapine is administered to post-treat at least 270 minutes post to the (S)-methylone. In some embodiments, the serotonin receptor modulator is quetiapine and the psychedelic is (S)-methylone, wherein the quetiapine is administered to post-treat at least 300 minutes post to the (S)-methylone. In some embodiments, the serotonin receptor modulator is quetiapine and the psychedelic is (S)-methylone, wherein the quetiapine is administered to post-treat at least 330 minutes post to the (S)-methylone. In some embodiments, the serotonin receptor modulator is quetiapine and the psychedelic is (S)-methylone, wherein the quetiapine is administered to post-treat at least 360 minutes post to the (S)-methylone. In some preferred embodiments, the serotonin receptor modulator is quetiapine and the psychedelic is (S)-methylone, wherein quetiapine is administered to post-treat between about 60 minutes and about 180 minutes post to the administration of (S)-methylone.

In some embodiments, the serotonin receptor modulator is risperidone and the psychedelic is (S)-methylone, wherein the risperidone is administered to post-treat at least 15 minutes post to the administration of (S)-methylone. In some embodiments, the serotonin receptor modulator is risperidone and the psychedelic is (S)-methylone, wherein the risperidone is administered to post-treat at least 30 minutes post to the (S)-methylone. In some embodiments, the serotonin receptor modulator is risperidone and the psychedelic is (S)-methylone, wherein the risperidone is administered to post-treat at least 60 minutes post to the (S)-methylone. In some embodiments, the serotonin receptor modulator is risperidone and the psychedelic is (S)-methylone, wherein the risperidone is administered to post-treat at least 90 minutes post to the (S)-methylone. In some embodiments, the serotonin receptor modulator is risperidone and the psychedelic is (S)-methylone, wherein the risperidone is administered to post-treat at least 120 minutes post to the (S)-methylone. In some embodiments, the serotonin receptor modulator is risperidone and the psychedelic is (S)-methylone, wherein the risperidone is administered to post-treat at least 150 minutes post to the (S)-methylone. In some embodiments, the serotonin receptor modulator is risperidone and the psychedelic is (S)-methylone, wherein the risperidone is administered to post-treat between about 15 minutes and about 150 minutes post to the (S)-methylone. In some embodiments, the serotonin receptor modulator is risperidone and the psychedelic is (S)-methylone, wherein the risperidone is administered to post-treat at least 180 minutes post to the (S)-methylone. In some embodiments, the serotonin receptor modulator is risperidone and the psychedelic is (S)-methylone, wherein the risperidone is administered to post-treat at least 210 minutes post to the (S)-methylone. In some embodiments, the serotonin receptor modulator is risperidone and the psychedelic is (S)-methylone, wherein the risperidone is administered to post-treat at least 240 minutes post to the (S)-methylone. In some embodiments, the serotonin receptor modulator is risperidone and the psychedelic is (S)-methylone, wherein the risperidone is administered to post-treat at least 270 minutes post to the (S)-methylone. In some embodiments, the serotonin receptor modulator is risperidone and the psychedelic is (S)-methylone, wherein the risperidone is administered to post-treat at least 300 minutes post to the (S)-methylone. In some embodiments, the serotonin receptor modulator is risperidone and the psychedelic is (S)-methylone, wherein the risperidone is administered to post-treat at least 330 minutes post to the (S)-methylone. In some embodiments, the serotonin receptor modulator is risperidone and the psychedelic is (S)-methylone, wherein the risperidone is administered to post-treat at least 360 minutes post to the (S)-methylone. In some preferred embodiments, the serotonin receptor modulator is risperidone and the psychedelic is (S)-methylone, wherein risperidone is administered to post-treat between about 60 minutes and about 180 minutes post to the administration of (S)-methylone.

In some embodiments, the serotonin receptor modulator is eplivanserin and the psychedelic is N-ethyl-3,4-methylenedioxyamphetamine (MDEA), wherein the eplivanserin is administered to post-treat at least 15 minutes post to the administration of N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 30 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 60 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 90 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat between at least 30 minutes post and 360 minutes post to the release or administration of the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat between at least 60 minutes post and 360 minutes post to the release or administration the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat between at least 90 minutes and 240 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 120 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 150 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat between about 15 minutes and about 150 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 180 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 210 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 240 minutes post to the N-ethyl-3,4-methylenedioxyamphetamine (MDEA).

In some embodiments, the serotonin receptor modulator is eplivanserin and the psychedelic is N-ethyl-3,4-methylenedioxyamphetamine (MDEA), wherein the eplivanserin is administered to post-treat at least 270 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 300 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 330 minutes post to the N-ethyl-3,4-methylenedioxyamphetamine (MDEA). In some embodiments, the serotonin receptor modulator is eplivanserin and the psychedelic is N-ethyl-3,4-methylenedioxyamphetamine (MDEA), wherein the eplivanserin is administered to post-treat at least 360 minutes post to the N-ethyl-3,4-methylenedioxyamphetamine (MDEA).

In some preferred embodiments, the serotonin receptor modulator is eplivanserin and the psychedelic is N-ethyl-3,4-methylenedioxyamphetamine (MDEA), wherein eplivanserin is administered to post-treat between about 60 minutes and about 180 minutes post to the administration of N-ethyl-3,4-methylenedioxyamphetamine (MDEA).

In some embodiments, the serotonin receptor modulator is volinanserin and the psychedelic is N-ethyl-3,4-methylenedioxyamphetamine (MDEA), wherein the volinanserin is administered to post-treat at least 15 minutes post to the administration of N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 30 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 60 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat a subject between at least 15 minutes and 360 minutes post to the administration or release of N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat between at least 30 minutes and 360 minutes post to the administration or release of N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat between at least 60 minutes and 240 minutes post to the administration or release of N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 90 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 120 minutes post to the N-ethyl-3,4-methylenedioxyamphetamine (MDEA).

In some embodiments, the serotonin receptor modulator is volinanserin and the psychedelic is N-ethyl-3,4-methylenedioxyamphetamine (MDEA), wherein the volinanserin is administered to post-treat at least 150 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat between about 15 minutes and about 150 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 180 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 210 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 240 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 270 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 300 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 330 minutes post to the N-ethyl-3,4-methylenedioxyamphetamine (MDEA). In some embodiments, the serotonin receptor modulator is volinanserin and the psychedelic is N-ethyl-3,4-methylenedioxyamphetamine (MDEA), wherein the volinanserin is administered to post-treat at least 360 minutes post to the N-ethyl-3,4-methylenedioxyamphetamine (MDEA). In some preferred embodiments, the serotonin receptor modulator is volinanserin and the psychedelic is N-ethyl-3,4-methylenedioxyamphetamine (MDEA), wherein volinanserin is administered to post-treat between about 60 minutes and about 180 minutes post to the administration of N-ethyl-3,4-methylenedioxyamphetamine (MDEA).

In some embodiments, the serotonin receptor modulator is ketanserin and the psychedelic is N-ethyl-3,4-methylenedioxyamphetamine (MDEA), wherein the ketanserin is administered to post-treat at least 15 minutes post to the administration of N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 30 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 60 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat between at least 30 minutes and 360 minutes post to the administration or release N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat between at least 60 minutes and 240 minutes post to the administration or release of N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 90 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 120 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 150 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat between about 15 minutes and about 150 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 180 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 210 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 240 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 270 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 300 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 330 minutes post to the N-ethyl-3,4-methylenedioxyamphetamine (MDEA). In some embodiments, the serotonin receptor modulator is ketanserin and the psychedelic is N-ethyl-3,4-methylenedioxyamphetamine (MDEA), wherein the ketanserin is administered to post-treat at least 360 minutes post to the N-ethyl-3,4-methylenedioxyamphetamine (MDEA). In some preferred embodiments, the serotonin receptor modulator is ketanserin and the psychedelic is N-ethyl-3,4-methylenedioxyamphetamine (MDEA), wherein ketanserin is administered to post-treat between about 60 minutes and about 180 minutes post to the administration of N-ethyl-3,4-methylenedioxyamphetamine (MDEA).

In some embodiments, the serotonin receptor modulator is ritanserin and the psychedelic is N-ethyl-3,4-methylenedioxyamphetamine (MDEA), wherein the ritanserin is administered to post-treat at least 15 minutes post to the administration of N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 30 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 60 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat between at least 60 minutes and 240 minutes post to the administration or release of N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 90 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 120 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 150 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat between about 15 minutes and about 150 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 180 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 210 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 240 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 270 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 300 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 330 minutes post to the N-ethyl-3,4-methylenedioxyamphetamine (MDEA). In some embodiments, the serotonin receptor modulator is ritanserin and the psychedelic is N-ethyl-3,4-methylenedioxyamphetamine (MDEA), wherein the ritanserin is administered to post-treat at least 360 minutes post to the N-ethyl-3,4-methylenedioxyamphetamine (MDEA). In some preferred embodiments, the serotonin receptor modulator is ritanserin and the psychedelic is N-ethyl-3,4-methylenedioxyamphetamine (MDEA), wherein ritanserin is administered to post-treat between about 60 minutes and about 180 minutes post to the administration of N-ethyl-3,4-methylenedioxyamphetamine (MDEA).

In some embodiments, the serotonin receptor modulator is pimavanserin and the psychedelic is N-ethyl-3,4-methylenedioxyamphetamine (MDEA), wherein the pimavanserin is administered to post-treat at least 15 minutes post to the administration of N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 30 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 60 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat between at least 60 minutes and 240 minutes post to the administration or release of N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 90 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 120 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 150 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat between about 15 minutes and about 150 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 180 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 210 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 240 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 270 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 300 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 330 minutes post to the N-ethyl-3,4-methylenedioxyamphetamine (MDEA). In some embodiments, the serotonin receptor modulator is pimavanserin and the psychedelic is N-ethyl-3,4-methylenedioxyamphetamine (MDEA), wherein the pimavanserin is administered to post-treat at least 360 minutes post to the N-ethyl-3,4-methylenedioxyamphetamine (MDEA). In some preferred embodiments, the serotonin receptor modulator is pimavanserin and the psychedelic is N-ethyl-3,4-methylenedioxyamphetamine (MDEA), wherein pimavanserin is administered to post-treat between about 60 minutes and about 180 minutes post to the administration of N-ethyl-3,4-methylenedioxyamphetamine (MDEA).

In some embodiments, the serotonin receptor modulator is nelotanserin and the psychedelic is N-ethyl-3,4-methylenedioxyamphetamine (MDEA), wherein the nelotanserin is administered to post-treat at least 15 minutes post to the administration of N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 30 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 60 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat between at least 60 minutes and 240 minutes post to the administration or release of N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 90 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 120 minutes post to the N-ethyl-3,4-methylenedioxyamphetamine (MDEA).

In some embodiments, the serotonin receptor modulator is nelotanserin and the psychedelic is N-ethyl-3,4-methylenedioxyamphetamine (MDEA), wherein the nelotanserin is administered to post-treat at least 150 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat between about 15 minutes and about 150 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 180 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 210 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 240 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 270 minutes post to the N-ethyl-3,4-methylenedioxyamphetamine (MDEA).

In some embodiments, the serotonin receptor modulator is nelotanserin and the psychedelic is N-ethyl-3,4-methylenedioxyamphetamine (MDEA), wherein the nelotanserin is administered to post-treat at least 300 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 330 minutes post to the N-ethyl-3,4-methylenedioxyamphetamine (MDEA). In some embodiments, the serotonin receptor modulator is nelotanserin and the psychedelic is N-ethyl-3,4-methylenedioxyamphetamine (MDEA), wherein the nelotanserin is administered to post-treat at least 360 minutes post to the N-ethyl-3,4-methylenedioxyamphetamine (MDEA). In some preferred embodiments, the serotonin receptor modulator is nelotanserin and the psychedelic is N-ethyl-3,4-methylenedioxyamphetamine (MDEA), wherein nelotanserin is administered to post-treat between about 60 minutes and about 180 minutes post to the administration of N-ethyl-3,4-methylenedioxyamphetamine (MDEA).

In some embodiments, the serotonin receptor modulator is pruvanserin and the psychedelic is N-ethyl-3,4-methylenedioxyamphetamine (MDEA), wherein the pruvanserin is administered to post-treat at least 15 minutes post to the administration of N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 30 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 60 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat between at least 60 minutes and 240 minutes post to the administration or release of N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 90 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 120 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 150 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat between about 15 minutes and about 150 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 180 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 210 minutes post to the N-ethyl-3,4-methylenedioxyamphetamine (MDEA).

In some embodiments, the serotonin receptor modulator is pruvanserin and the psychedelic is N-ethyl-3,4-methylenedioxyamphetamine (MDEA), wherein the pruvanserin is administered to post-treat at least 240 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 270 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 300 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 330 minutes post to the N-ethyl-3,4-methylenedioxyamphetamine (MDEA). In some embodiments, the serotonin receptor modulator is pruvanserin and the psychedelic is N-ethyl-3,4-methylenedioxyamphetamine (MDEA), wherein the pruvanserin is administered to post-treat at least 360 minutes post to the N-ethyl-3,4-methylenedioxyamphetamine (MDEA). In some preferred embodiments, the serotonin receptor modulator is pruvanserin and the psychedelic is N-ethyl-3,4-methylenedioxyamphetamine (MDEA), wherein pruvanserin is administered to post-treat between about 60 minutes and about 180 minutes post to the administration of N-ethyl-3,4-methylenedioxyamphetamine (MDEA).

In some embodiments, the serotonin receptor modulator is flibanserin and the psychedelic is N-ethyl-3,4-methylenedioxyamphetamine (MDEA), wherein the flibanserin is administered to post-treat at least 15 minutes post to the administration of N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 30 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 60 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 90 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 120 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 150 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat between about 15 minutes and about 150 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 180 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 210 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 240 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 270 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 300 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 330 minutes post to the N-ethyl-3,4-methylenedioxyamphetamine (MDEA). In some embodiments, the serotonin receptor modulator is flibanserin and the psychedelic is N-ethyl-3,4-methylenedioxyamphetamine (MDEA), wherein the flibanserin is administered to post-treat at least 360 minutes post to the N-ethyl-3,4-methylenedioxyamphetamine (MDEA). In some preferred embodiments, the serotonin receptor modulator is flibanserin and the psychedelic is N-ethyl-3,4-methylenedioxyamphetamine (MDEA), wherein flibanserin is administered to post-treat between about 60 minutes and about 180 minutes post to the administration of N-ethyl-3,4-methylenedioxyamphetamine (MDEA).

In some embodiments, the serotonin receptor modulator is olanzapine and the psychedelic is N-ethyl-3,4-methylenedioxyamphetamine (MDEA), wherein the olanzapine is administered to post-treat at least 15 minutes post to the administration of N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 30 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 60 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 90 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 120 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 150 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat between about 15 minutes and about 150 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 180 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 210 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 240 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 270 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 300 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 330 minutes post to the N-ethyl-3,4-methylenedioxyamphetamine (MDEA). In some embodiments, the serotonin receptor modulator is olanzapine and the psychedelic is N-ethyl-3,4-methylenedioxyamphetamine (MDEA), wherein the olanzapine is administered to post-treat at least 360 minutes post to the N-ethyl-3,4-methylenedioxyamphetamine (MDEA). In some preferred embodiments, the serotonin receptor modulator is olanzapine and the psychedelic is N-ethyl-3,4-methylenedioxyamphetamine (MDEA), wherein olanzapine is administered to post-treat between about 60 minutes and about 180 minutes post to the administration of N-ethyl-3,4-methylenedioxyamphetamine (MDEA).

In some embodiments, the serotonin receptor modulator is quetiapine and the psychedelic is N-ethyl-3,4-methylenedioxyamphetamine (MDEA), wherein the quetiapine is administered to post-treat at least 15 minutes post to the administration of N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 30 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 60 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 90 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 120 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 150 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat between about 15 minutes and about 150 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 180 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 210 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 240 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 270 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 300 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 330 minutes post to the N-ethyl-3,4-methylenedioxyamphetamine (MDEA). In some embodiments, the serotonin receptor modulator is quetiapine and the psychedelic is N-ethyl-3,4-methylenedioxyamphetamine (MDEA), wherein the quetiapine is administered to post-treat at least 360 minutes post to the N-ethyl-3,4-methylenedioxyamphetamine (MDEA). In some preferred embodiments, the serotonin receptor modulator is quetiapine and the psychedelic is N-ethyl-3,4-methylenedioxyamphetamine (MDEA), wherein quetiapine is administered to post-treat between about 60 minutes and about 180 minutes post to the administration of N-ethyl-3,4-methylenedioxyamphetamine (MDEA).

In some embodiments, the serotonin receptor modulator is risperidone and the psychedelic is N-ethyl-3,4-methylenedioxyamphetamine (MDEA), wherein the risperidone is administered to post-treat at least 15 minutes post to the administration of N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 30 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 60 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 90 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 120 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 150 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat between about 15 minutes and about 150 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 180 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 210 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 240 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 270 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 300 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 330 minutes post to the N-ethyl-3,4-methylenedioxyamphetamine (MDEA). In some embodiments, the serotonin receptor modulator is risperidone and the psychedelic is N-ethyl-3,4-methylenedioxyamphetamine (MDEA), wherein the risperidone is administered to post-treat at least 360 minutes post to the N-ethyl-3,4-methylenedioxyamphetamine (MDEA). In some preferred embodiments, the serotonin receptor modulator is risperidone and the psychedelic is N-ethyl-3,4-methylenedioxyamphetamine (MDEA), wherein risperidone is administered to post-treat between about 60 minutes and about 180 minutes post to the administration of N-ethyl-3,4-methylenedioxyamphetamine (MDEA).

In some embodiments, the serotonin receptor modulator is eplivanserin and the psychedelic is (S)—N-ethyl-3,4-methylenedioxyamphetamine (S-MDEA), wherein the eplivanserin is administered to post-treat at least 15 minutes post to the administration of (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 30 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 60 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 90 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat between at least 30 minutes post and 360 minutes post to the release or administration of the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat between at least 60 minutes post and 360 minutes post to the release or administration the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat between at least 90 minutes and 240 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 120 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 150 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat between about 15 minutes and about 150 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 180 minutes post to the (S)—N-ethyl-3,4-methylenedioxy amphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 210 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 240 minutes post to the (S)—N-ethyl-3,4-methylenedioxyamphetamine (S-MDEA).

In some embodiments, the serotonin receptor modulator is eplivanserin and the psychedelic is (S)—N-ethyl-3,4-methylenedioxyamphetamine (S-MDEA), wherein the eplivanserin is administered to post-treat at least 270 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 300 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 330 minutes post to the (S)—N-ethyl-3,4-methylenedioxyamphetamine (S-MDEA). In some embodiments, the serotonin receptor modulator is eplivanserin and the psychedelic is (S)—N-ethyl-3,4-methylenedioxyamphetamine (S-MDEA), wherein the eplivanserin is administered to post-treat at least 360 minutes post to the (S)—N-ethyl-3,4-methylenedioxy amphetamine (S-MDEA).

In some preferred embodiments, the serotonin receptor modulator is eplivanserin and the psychedelic is (S)—N-ethyl-3,4-methylenedioxyamphetamine (S-MDEA), wherein eplivanserin is administered to post-treat between about 60 minutes and about 180 minutes post to the administration of (S)—N-ethyl-3,4-methylenedioxyamphetamine (S-MDEA).

In some embodiments, the serotonin receptor modulator is volinanserin and the psychedelic is (S)—N-ethyl-3,4-methylenedioxyamphetamine (S-MDEA), wherein the volinanserin is administered to post-treat at least 15 minutes post to the administration of (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 30 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 60 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat a subject between at least 15 minutes and 360 minutes post to the administration or release of (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat between at least 30 minutes and 360 minutes post to the administration or release of (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat between at least 60 minutes and 240 minutes post to the administration or release of (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 90 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 120 minutes post to the (S)—N-ethyl-3,4-methylenedioxy amphetamine (S-MDEA).

In some embodiments, the serotonin receptor modulator is volinanserin and the psychedelic is (S)—N-ethyl-3,4-methylenedioxyamphetamine (S-MDEA), wherein the volinanserin is administered to post-treat at least 150 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat between about 15 minutes and about 150 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 180 minutes post to the (S)—N-ethyl-3,4-methylenedioxy amphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 210 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 240 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 270 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 300 minutes post to the (S)—N-ethyl-3,4-methylenedioxy amphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 330 minutes post to the (S)—N-ethyl-3,4-methylenedioxyamphetamine (S-MDEA). In some embodiments, the serotonin receptor modulator is volinanserin and the psychedelic is (S)—N-ethyl-3,4-methylenedioxyamphetamine (S-MDEA), wherein the volinanserin is administered to post-treat at least 360 minutes post to the (S)—N-ethyl-3,4-methylenedioxyamphetamine (S-MDEA). In some preferred embodiments, the serotonin receptor modulator is volinanserin and the psychedelic is (S)—N-ethyl-3,4-methylenedioxyamphetamine (S-MDEA), wherein volinanserin is administered to post-treat between about 60 minutes and about 180 minutes post to the administration of (S)—N-ethyl-3,4-methylenedioxyamphetamine (S-MDEA).

In some embodiments, the serotonin receptor modulator is ketanserin and the psychedelic is (S)—N-ethyl-3,4-methylenedioxyamphetamine (S-MDEA), wherein the ketanserin is administered to post-treat at least 15 minutes post to the administration of (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 30 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 60 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat between at least 30 minutes and 360 minutes post to the administration or release (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat between at least 60 minutes and 240 minutes post to the administration or release of (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 90 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 120 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 150 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat between about 15 minutes and about 150 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 180 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 210 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 240 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 270 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 300 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 330 minutes post to the (S)—N-ethyl-3,4-methylenedioxyamphetamine (S-MDEA). In some embodiments, the serotonin receptor modulator is ketanserin and the psychedelic is (S)—N-ethyl-3,4-methylenedioxyamphetamine (S-MDEA), wherein the ketanserin is administered to post-treat at least 360 minutes post to the (S)—N-ethyl-3,4-methylenedioxyamphetamine (S-MDEA). In some preferred embodiments, the serotonin receptor modulator is ketanserin and the psychedelic is (S)—N-ethyl-3,4-methylenedioxyamphetamine (S-MDEA), wherein ketanserin is administered to post-treat between about 60 minutes and about 180 minutes post to the administration of (S)—N-ethyl-3,4-methylenedioxyamphetamine (S-MDEA).

In some embodiments, the serotonin receptor modulator is ritanserin and the psychedelic is (S)—N-ethyl-3,4-methylenedioxyamphetamine (S-MDEA), wherein the ritanserin is administered to post-treat at least 15 minutes post to the administration of (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 30 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 60 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat between at least 60 minutes and 240 minutes post to the administration or release of (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 90 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 120 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 150 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat between about 15 minutes and about 150 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 180 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 210 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 240 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 270 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 300 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 330 minutes post to the (S)—N-ethyl-3,4-methylenedioxyamphetamine (S-MDEA). In some embodiments, the serotonin receptor modulator is ritanserin and the psychedelic is (S)—N-ethyl-3,4-methylenedioxyamphetamine (S-MDEA), wherein the ritanserin is administered to post-treat at least 360 minutes post to the (S)—N-ethyl-3,4-methylenedioxyamphetamine (S-MDEA). In some preferred embodiments, the serotonin receptor modulator is ritanserin and the psychedelic is (S)—N-ethyl-3,4-methylenedioxyamphetamine (S-MDEA), wherein ritanserin is administered to post-treat between about 60 minutes and about 180 minutes post to the administration of (S)—N-ethyl-3,4-methylenedioxyamphetamine (S-MDEA).

In some embodiments, the serotonin receptor modulator is pimavanserin and the psychedelic is (S)—N-ethyl-3,4-methylenedioxyamphetamine (S-MDEA), wherein the pimavanserin is administered to post-treat at least 15 minutes post to the administration of (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 30 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 60 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat between at least 60 minutes and 240 minutes post to the administration or release of (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 90 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 120 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 150 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat between about 15 minutes and about 150 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 180 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 210 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 240 minutes post to the (S)—N-ethyl-3,4-methylenedioxy amphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 270 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 300 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 330 minutes post to the (S)—N-ethyl-3,4-methylenedioxyamphetamine (S-MDEA). In some embodiments, the serotonin receptor modulator is pimavanserin and the psychedelic is (S)—N-ethyl-3,4-methylenedioxyamphetamine (S-MDEA), wherein the pimavanserin is administered to post-treat at least 360 minutes post to the (S)—N-ethyl-3,4-methylenedioxy amphetamine (S-MDEA). In some preferred embodiments, the serotonin receptor modulator is pimavanserin and the psychedelic is (S)—N-ethyl-3,4-methylenedioxyamphetamine (S-MDEA), wherein pimavanserin is administered to post-treat between about 60 minutes and about 180 minutes post to the administration of (S)—N-ethyl-3,4-methylenedioxy amphetamine (S-MDEA).

In some embodiments, the serotonin receptor modulator is nelotanserin and the psychedelic is (S)—N-ethyl-3,4-methylenedioxyamphetamine (S-MDEA), wherein the nelotanserin is administered to post-treat at least 15 minutes post to the administration of (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 30 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 60 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat between at least 60 minutes and 240 minutes post to the administration or release of (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 90 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 120 minutes post to the (S)—N-ethyl-3,4-methylenedioxyamphetamine (S-MDEA).

In some embodiments, the serotonin receptor modulator is nelotanserin and the psychedelic is (S)—N-ethyl-3,4-methylenedioxyamphetamine (S-MDEA), wherein the nelotanserin is administered to post-treat at least 150 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat between about 15 minutes and about 150 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 180 minutes post to the (S)—N-ethyl-3,4-methylenedioxy amphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 210 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 240 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 270 minutes post to the (S)—N-ethyl-3,4-methylenedioxyamphetamine (S-MDEA).

In some embodiments, the serotonin receptor modulator is nelotanserin and the psychedelic is (S)—N-ethyl-3,4-methylenedioxyamphetamine (S-MDEA), wherein the nelotanserin is administered to post-treat at least 300 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 330 minutes post to the (S)—N-ethyl-3,4-methylenedioxyamphetamine (S-MDEA). In some embodiments, the serotonin receptor modulator is nelotanserin and the psychedelic is (S)—N-ethyl-3,4-methylenedioxyamphetamine (S-MDEA), wherein the nelotanserin is administered to post-treat at least 360 minutes post to the (S)—N-ethyl-3,4-methylenedioxyamphetamine (S-MDEA). In some preferred embodiments, the serotonin receptor modulator is nelotanserin and the psychedelic is (S)—N-ethyl-3,4-methylenedioxyamphetamine (S-MDEA), wherein nelotanserin is administered to post-treat between about 60 minutes and about 180 minutes post to the administration of (S)—N-ethyl-3,4-methylenedioxyamphetamine (S-MDEA).

In some embodiments, the serotonin receptor modulator is pruvanserin and the psychedelic is (S)—N-ethyl-3,4-methylenedioxyamphetamine (S-MDEA), wherein the pruvanserin is administered to post-treat at least 15 minutes post to the administration of (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 30 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 60 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat between at least 60 minutes and 240 minutes post to the administration or release of (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 90 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 120 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 150 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat between about 15 minutes and about 150 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 180 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 210 minutes post to the (S)—N-ethyl-3,4-methylenedioxyamphetamine (S-MDEA).

In some embodiments, the serotonin receptor modulator is pruvanserin and the psychedelic is (S)—N-ethyl-3,4-methylenedioxyamphetamine (S-MDEA), wherein the pruvanserin is administered to post-treat at least 240 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 270 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 300 minutes post to the (S)—N-ethyl-3,4-methylenedioxyamphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 330 minutes post to the (S)—N-ethyl-3,4-methylenedioxy amphetamine (S-MDEA). In some embodiments, the serotonin receptor modulator is pruvanserin and the psychedelic is (S)—N-ethyl-3,4-methylenedioxyamphetamine (S-MDEA), wherein the pruvanserin is administered to post-treat at least 360 minutes post to the (S)—N-ethyl-3,4-methylenedioxyamphetamine (S-MDEA). In some preferred embodiments, the serotonin receptor modulator is pruvanserin and the psychedelic is (S)—N-ethyl-3,4-methylenedioxyamphetamine (S-MDEA), wherein pruvanserin is administered to post-treat between about 60 minutes and about 180 minutes post to the administration of (S)—N-ethyl-3,4-methylenedioxyamphetamine (S-MDEA).

In some embodiments, the serotonin receptor modulator is flibanserin and the psychedelic is (S)—N-ethyl-3,4-methylenedioxyamphetamine (S-MDEA), wherein the flibanserin is administered to post-treat at least 15 minutes post to the administration of (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 30 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 60 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 90 minutes post to the (S)—N-ethyl-3,4-methylenedioxy amphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 120 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 150 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat between about 15 minutes and about 150 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 180 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 210 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 240 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 270 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 300 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 330 minutes post to the (S)—N-ethyl-3,4-methylenedioxyamphetamine (S-MDEA). In some embodiments, the serotonin receptor modulator is flibanserin and the psychedelic is (S)—N-ethyl-3,4-methylenedioxyamphetamine (S-MDEA), wherein the flibanserin is administered to post-treat at least 360 minutes post to the (S)—N-ethyl-3,4-methylenedioxyamphetamine (S-MDEA). In some preferred embodiments, the serotonin receptor modulator is flibanserin and the psychedelic is (S)—N-ethyl-3,4-methylenedioxyamphetamine (S-MDEA), wherein flibanserin is administered to post-treat between about 60 minutes and about 180 minutes post to the administration of (S)—N-ethyl-3,4-methylenedioxyamphetamine (S-MDEA).

In some embodiments, the serotonin receptor modulator is olanzapine and the psychedelic is (S)—N-ethyl-3,4-methylenedioxyamphetamine (S-MDEA), wherein the olanzapine is administered to post-treat at least 15 minutes post to the administration of (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 30 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 60 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 90 minutes post to the (S)—N-ethyl-3,4-methylenedioxy amphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 120 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 150 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat between about 15 minutes and about 150 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 180 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 210 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 240 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 270 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 300 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 330 minutes post to the (S)—N-ethyl-3,4-methylenedioxyamphetamine (S-MDEA). In some embodiments, the serotonin receptor modulator is olanzapine and the psychedelic is (S)—N-ethyl-3,4-methylenedioxyamphetamine (S-MDEA), wherein the olanzapine is administered to post-treat at least 360 minutes post to the (S)—N-ethyl-3,4-methylenedioxyamphetamine (S-MDEA). In some preferred embodiments, the serotonin receptor modulator is olanzapine and the psychedelic is (S)—N-ethyl-3,4-methylenedioxyamphetamine (S-MDEA), wherein olanzapine is administered to post-treat between about 60 minutes and about 180 minutes post to the administration of (S)—N-ethyl-3,4-methylenedioxyamphetamine (S-MDEA).

In some embodiments, the serotonin receptor modulator is quetiapine and the psychedelic is (S)—N-ethyl-3,4-methylenedioxyamphetamine (S-MDEA), wherein the quetiapine is administered to post-treat at least 15 minutes post to the administration of (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 30 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 60 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 90 minutes post to the (S)—N-ethyl-3,4-methylenedioxy amphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 120 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 150 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat between about 15 minutes and about 150 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 180 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 210 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 240 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 270 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 300 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 330 minutes post to the (S)—N-ethyl-3,4-methylenedioxyamphetamine (S-MDEA). In some embodiments, the serotonin receptor modulator is quetiapine and the psychedelic is (S)—N-ethyl-3,4-methylenedioxyamphetamine (S-MDEA), wherein the quetiapine is administered to post-treat at least 360 minutes post to the (S)—N-ethyl-3,4-methylenedioxyamphetamine (S-MDEA). In some preferred embodiments, the serotonin receptor modulator is quetiapine and the psychedelic is (S)—N-ethyl-3,4-methylenedioxyamphetamine (S-MDEA), wherein quetiapine is administered to post-treat between about 60 minutes and about 180 minutes post to the administration of (S)—N-ethyl-3,4-methylenedioxyamphetamine (S-MDEA).

In some embodiments, the serotonin receptor modulator is risperidone and the psychedelic is (S)—N-ethyl-3,4-methylenedioxyamphetamine (S-MDEA), wherein the risperidone is administered to post-treat at least 15 minutes post to the administration of(S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 30 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 60 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 90 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 120 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 150 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat between about 15 minutes and about 150 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 180 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 210 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 240 minutes post to the (S)—N-ethyl-3,4-methylenedioxy amphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 270 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 300 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 330 minutes post to the (S)—N-ethyl-3,4-methylenedioxyamphetamine (S-MDEA). In some embodiments, the serotonin receptor modulator is risperidone and the psychedelic is (S)—N-ethyl-3,4-methylenedioxyamphetamine (S-MDEA), wherein the risperidone is administered to post-treat at least 360 minutes post to the (S)—N-ethyl-3,4-methylenedioxy amphetamine (S-MDEA). In some preferred embodiments, the serotonin receptor modulator is risperidone and the psychedelic is (S)—N-ethyl-3,4-methylenedioxyamphetamine (S-MDEA), wherein risperidone is administered to post-treat between about 60 minutes and about 180 minutes post to the administration of (S)—N-ethyl-3,4-methylenedioxy amphetamine (S-MDEA).

In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat at least 15 minutes post to the administration of N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 30 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 60 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat at least 90 minutes post to the N-ethyl-2-(5-fluoro-TH-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat between at least 30 minutes post and 360 minutes post to the release or administration of the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat between at least 60 minutes post and 360 minutes post to the release or administration the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat between at least 90 minutes and 240 minutes post to the N-ethyl-2-(5-fluoro-TH-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 120 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 150 minutes post to the N-ethyl-2-(5-fluoro-TH-indol-3-yl)-N-methylethan-1-amine. In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat between about 15 minutes and about 150 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 180 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 210 minutes post to the N-ethyl-2-(5-fluoro-TH-indol-3-yl)-N-methylethan-1-amine. In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat at least 240 minutes post to the N-ethyl-2-(5-fluoro-TH-indol-3-yl)-N-methylethan-1-amine.

In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 270 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-1H-indol-3-yl)-N-methylethan-1-amine, wherein the eplivanserin is administered to post-treat at least 300 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 330 minutes post to the N-ethyl-2-(5-fluoro-1H-indol-3-yl)-N-methylethan-1-amine. In some embodiments, the serotonin receptor modulator is eplivanserin and the psychedelic is N-ethyl-2-(5-fluoro-TH-indol-3-yl)-N-methylethan-1-amine, wherein the eplivanserin is administered to post-treat at least 360 minutes post to the N-ethyl-2-(5-fluoro-1H-indol-3-yl)-N-methylethan-1-amine.

In some preferred embodiments, the serotonin receptor modulator is eplivanserin and the psychedelic is N-ethyl-2-(5-fluoro-TH-indol-3-yl)-N-methylethan-1-amine, wherein eplivanserin is administered to post-treat between about 60 minutes and about 180 minutes post to the administration of N-ethyl-2-(5-fluoro-TH-indol-3-yl)-N-methylethan-1-amine.

In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 15 minutes post to the administration of N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 30 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 60 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat a subject between at least 15 minutes and 360 minutes post to the administration or release of N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat between at least 30 minutes and 360 minutes post to the administration or release of N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat between at least 60 minutes and 240 minutes post to the administration or release of N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 90 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 120 minutes post to the N-ethyl-2-(5-fluoro-1H-indol-3-yl)-N-methylethan-1-amine.

In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 150 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat between about 15 minutes and about 150 minutes post to the N-ethyl-2-(5-fluoro-TH-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 180 minutes post to the N-ethyl-2-(5-fluoro-TH-indol-3-yl)-N-methylethan-1-amine. In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 210 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 240 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 270 minutes post to the N-ethyl-2-(5-fluoro-1H-indol-3-yl)-N-methylethan-1-amine. In some embodiments, the serotonin receptor modulator is volinanserin and the psychedelic is N-ethyl-2-(5-fluoro-TH-indol-3-yl)-N-methylethan-1-amine, wherein the volinanserin is administered to post-treat at least 300 minutes post to the N-ethyl-2-(5-fluoro-TH-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 330 minutes post to the N-ethyl-2-(5-fluoro-TH-indol-3-yl)-N-methylethan-1-amine. In some embodiments, the serotonin receptor modulator is volinanserin and the psychedelic is N-ethyl-2-(5-fluoro-1H-indol-3-yl)-N-methylethan-1-amine, wherein the volinanserin is administered to post-treat at least 360 minutes post to the N-ethyl-2-(5-fluoro-1H-indol-3-yl)-N-methylethan-1-amine. In some preferred embodiments, the serotonin receptor modulator is volinanserin and the psychedelic is N-ethyl-2-(5-fluoro-TH-indol-3-yl)-N-methylethan-1-amine, wherein volinanserin is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 15 minutes post to the administration of N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 30 minutes post to the N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 60 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat between at least 30 minutes and 360 minutes post to the administration or release N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat between at least 60 minutes and 240 minutes post to the administration or release of N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 90 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 120 minutes post to the N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 150 minutes post to the N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-1H-indol-3-yl)-N-methylethan-1-amine, wherein the ketanserin is administered to post-treat between about 15 minutes and about 150 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 180 minutes post to the N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-1H-indol-3-yl)-N-methylethan-1-amine, wherein the ketanserin is administered to post-treat at least 210 minutes post to the N-ethyl-2-(5-fluoro-1H-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 240 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 270 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 300 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 330 minutes post to the N-ethyl-2-(5-fluoro-TH-indol-3-yl)-N-methylethan-1-amine. In some embodiments, the serotonin receptor modulator is ketanserin and the psychedelic is N-ethyl-2-(5-fluoro-TH-indol-3-yl)-N-methylethan-1-amine, wherein the ketanserin is administered to post-treat at least 360 minutes post to the N-ethyl-2-(5-fluoro-TH-indol-3-yl)-N-methylethan-1-amine. In some preferred embodiments, the serotonin receptor modulator is ketanserin and the psychedelic is N-ethyl-2-(5-fluoro-TH-indol-3-yl)-N-methylethan-1-amine, wherein ketanserin is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 15 minutes post to the administration of N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 30 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-1H-indol-3-yl)-N-methylethan-1-amine, wherein the ritanserin is administered to post-treat at least 60 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-1H-indol-3-yl)-N-methylethan-1-amine, wherein the ritanserin is administered to post-treat between at least 60 minutes and 240 minutes post to the administration or release of N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 90 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 120 minutes post to the N-ethyl-2-(5-fluoro-TH-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 150 minutes post to the N-ethyl-2-(5-fluoro-TH-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat between about 15 minutes and about 150 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 180 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 210 minutes post to the N-ethyl-2-(5-fluoro-TH-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 240 minutes post to the N-ethyl-2-(5-fluoro-TH-indol-3-yl)-N-methylethan-1-amine. In some embodiments, the serotonin receptor modulator is ritanserin and the psychedelic is N-ethyl-2-(5-fluoro-TH-indol-3-yl)-N-methylethan-1-amine, wherein the ritanserin is administered to post-treat at least 270 minutes post to the N-ethyl-2-(5-fluoro-TH-indol-3-yl)-N-methylethan-1-amine. In some embodiments, the serotonin receptor modulator is ritanserin and the psychedelic is N-ethyl-2-(5-fluoro-1H-indol-3-yl)-N-methylethan-1-amine, wherein the ritanserin is administered to post-treat at least 300 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-1H-indol-3-yl)-N-methylethan-1-amine, wherein the ritanserin is administered to post-treat at least 330 minutes post to the N-ethyl-2-(5-fluoro-1H-indol-3-yl)-N-methylethan-1-amine. In some embodiments, the serotonin receptor modulator is ritanserin and the psychedelic is N-ethyl-2-(5-fluoro-1H-indol-3-yl)-N-methylethan-1-amine, wherein the ritanserin is administered to post-treat at least 360 minutes post to the N-ethyl-2-(5-fluoro-1H-indol-3-yl)-N-methylethan-1-amine. In some preferred embodiments, the serotonin receptor modulator is ritanserin and the psychedelic is N-ethyl-2-(5-fluoro-TH-indol-3-yl)-N-methylethan-1-amine, wherein ritanserin is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 15 minutes post to the administration of N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 30 minutes post to the N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 60 minutes post to the N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat between at least 60 minutes and 240 minutes post to the administration or release of N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-1H-indol-3-yl)-N-methylethan-1-amine, wherein the pimavanserin is administered to post-treat at least 90 minutes post to the N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 120 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 150 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-1H-indol-3-yl)-N-methylethan-1-amine, wherein the pimavanserin is administered to post-treat between about 15 minutes and about 150 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-1H-indol-3-yl)-N-methylethan-1-amine, wherein the pimavanserin is administered to post-treat at least 180 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 210 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 240 minutes post to the N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 270 minutes post to the N-ethyl-2-(5-fluoro-TH-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 300 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 330 minutes post to the N-ethyl-2-(5-fluoro-1H-indol-3-yl)-N-methylethan-1-amine. In some embodiments, the serotonin receptor modulator is pimavanserin and the psychedelic is N-ethyl-2-(5-fluoro-TH-indol-3-yl)-N-methylethan-1-amine, wherein the pimavanserin is administered to post-treat at least 360 minutes post to the N-ethyl-2-(5-fluoro-TH-indol-3-yl)-N-methylethan-1-amine. In some preferred embodiments, the serotonin receptor modulator is pimavanserin and the psychedelic is N-ethyl-2-(5-fluoro-TH-indol-3-yl)-N-methylethan-1-amine, wherein pimavanserin is administered to post-treat between about 60 minutes and about 180 minutes post to the administration ofN-ethyl-2-(5-fluoro-1H-indol-3-yl)-N-methylethan-1-amine.

In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 15 minutes post to the administration of N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 30 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 60 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat between at least 60 minutes and 240 minutes post to the administration or release of N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 90 minutes post to the N-ethyl-2-(5-fluoro-TH-indol-3-yl)-N-methylethan-1-amine. In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 120 minutes post to the N-ethyl-2-(5-fluoro-1H-indol-3-yl)-N-methylethan-1-amine.

In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 150 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat between about 15 minutes and about 150 minutes post to the N-ethyl-2-(5-fluoro-TH-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 180 minutes post to the N-ethyl-2-(5-fluoro-TH-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 210 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 240 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 270 minutes post to the N-ethyl-2-(5-fluoro-1H-indol-3-yl)-N-methylethan-1-amine.

In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 300 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-1H-indol-3-yl)-N-methylethan-1-amine, wherein the nelotanserin is administered to post-treat at least 330 minutes post to the N-ethyl-2-(5-fluoro-1H-indol-3-yl)-N-methylethan-1-amine. In some embodiments, the serotonin receptor modulator is nelotanserin and the psychedelic is N-ethyl-2-(5-fluoro-TH-indol-3-yl)-N-methylethan-1-amine, wherein the nelotanserin is administered to post-treat at least 360 minutes post to the N-ethyl-2-(5-fluoro-1H-indol-3-yl)-N-methylethan-1-amine. In some preferred embodiments, the serotonin receptor modulator is nelotanserin and the psychedelic is N-ethyl-2-(5-fluoro-TH-indol-3-yl)-N-methylethan-1-amine, wherein nelotanserin is administered to post-treat between about 60 minutes and about 180 minutes post to the administration of N-ethyl-2-(5-fluoro-TH-indol-3-yl)-N-methylethan-1-amine.

In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 15 minutes post to the administration of N-ethyl-2-(5-fluoro-1H-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 30 minutes post to the N-ethyl-2-(5-fluoro-1H-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 60 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-1H-indol-3-yl)-N-methylethan-1-amine, wherein the pruvanserin is administered to post-treat between at least 60 minutes and 240 minutes post to the administration or release of N-ethyl-2-(5-fluoro-1H-indol-3-yl)-N-methylethan-1-amine. In some embodiments, the serotonin receptor modulator is pruvanserin and the psychedelic is N-ethyl-2-(5-fluoro-1H-indol-3-yl)-N-methylethan-1-amine, wherein the pruvanserin is administered to post-treat at least 90 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 120 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 150 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat between about 15 minutes and about 150 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 180 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 210 minutes post to the N-ethyl-2-(5-fluoro-TH-indol-3-yl)-N-methylethan-1-amine.

In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 240 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 270 minutes post to the N-ethyl-2-(5-fluoro-TH-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 300 minutes post to the N-ethyl-2-(5-fluoro-1H-indol-3-yl)-N-methylethan-1-amine. In some embodiments, the serotonin receptor modulator is pruvanserin and the psychedelic is N-ethyl-2-(5-fluoro-TH-indol-3-yl)-N-methylethan-1-amine, wherein the pruvanserin is administered to post-treat at least 330 minutes post to the N-ethyl-2-(5-fluoro-1H-indol-3-yl)-N-methylethan-1-amine. In some embodiments, the serotonin receptor modulator is pruvanserin and the psychedelic is N-ethyl-2-(5-fluoro-1H-indol-3-yl)-N-methylethan-1-amine, wherein the pruvanserin is administered to post-treat at least 360 minutes post to the N-ethyl-2-(5-fluoro-1H-indol-3-yl)-N-methylethan-1-amine. In some preferred embodiments, the serotonin receptor modulator is pruvanserin and the psychedelic is N-ethyl-2-(5-fluoro-TH-indol-3-yl)-N-methylethan-1-amine, wherein pruvanserin is administered to post-treat between about 60 minutes and about 180 minutes post to the administration of N-ethyl-2-(5-fluoro-TH-indol-3-yl)-N-methylethan-1-amine.

In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 15 minutes post to the administration of N-ethyl-2-(5-fluoro-1H-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 30 minutes post to the N-ethyl-2-(5-fluoro-1H-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 60 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 90 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 120 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 150 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat between about 15 minutes and about 150 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-1H-indol-3-yl)-N-methylethan-1-amine, wherein the flibanserin is administered to post-treat at least 180 minutes post to the N-ethyl-2-(5-fluoro-1H-indol-3-yl)-N-methylethan-1-amine. In some embodiments, the serotonin receptor modulator is flibanserin and the psychedelic is N-ethyl-2-(5-fluoro-1H-indol-3-yl)-N-methylethan-1-amine, wherein the flibanserin is administered to post-treat at least 210 minutes post to the N-ethyl-2-(5-fluoro-1H-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 240 minutes post to the N-ethyl-2-(5-fluoro-1H-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 270 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-1H-indol-3-yl)-N-methylethan-1-amine, wherein the flibanserin is administered to post-treat at least 300 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 330 minutes post to the N-ethyl-2-(5-fluoro-TH-indol-3-yl)-N-methylethan-1-amine. In some embodiments, the serotonin receptor modulator is flibanserin and the psychedelic is N-ethyl-2-(5-fluoro-TH-indol-3-yl)-N-methylethan-1-amine, wherein the flibanserin is administered to post-treat at least 360 minutes post to the N-ethyl-2-(5-fluoro-TH-indol-3-yl)-N-methylethan-1-amine. In some preferred embodiments, the serotonin receptor modulator is flibanserin and the psychedelic is N-ethyl-2-(5-fluoro-TH-indol-3-yl)-N-methylethan-1-amine, wherein flibanserin is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 15 minutes post to the administration of N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 30 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 60 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-1H-indol-3-yl)-N-methylethan-1-amine, wherein the olanzapine is administered to post-treat at least 90 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 120 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-1H-indol-3-yl)-N-methylethan-1-amine, wherein the olanzapine is administered to post-treat at least 150 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-1H-indol-3-yl)-N-methylethan-1-amine, wherein the olanzapine is administered to post-treat between about 15 minutes and about 150 minutes post to the N-ethyl-2-(5-fluoro-TH-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 180 minutes post to the N-ethyl-2-(5-fluoro-TH-indol-3-yl)-N-methylethan-1-amine. In some embodiments, the serotonin receptor modulator is olanzapine and the psychedelic is N-ethyl-2-(5-fluoro-1H-indol-3-yl)-N-methylethan-1-amine, wherein the olanzapine is administered to post-treat at least 210 minutes post to the N-ethyl-2-(5-fluoro-TH-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 240 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 270 minutes post to the N-ethyl-2-(5-fluoro-1H-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 300 minutes post to the N-ethyl-2-(5-fluoro-TH-indol-3-yl)-N-methylethan-1-amine. In some embodiments, the serotonin receptor modulator is olanzapine and the psychedelic is N-ethyl-2-(5-fluoro-TH-indol-3-yl)-N-methylethan-1-amine, wherein the olanzapine is administered to post-treat at least 330 minutes post to the N-ethyl-2-(5-fluoro-TH-indol-3-yl)-N-methylethan-1-amine. In some embodiments, the serotonin receptor modulator is olanzapine and the psychedelic is N-ethyl-2-(5-fluoro-1H-indol-3-yl)-N-methylethan-1-amine, wherein the olanzapine is administered to post-treat at least 360 minutes post to the N-ethyl-2-(5-fluoro-TH-indol-3-yl)-N-methylethan-1-amine. In some preferred embodiments, the serotonin receptor modulator is olanzapine and the psychedelic is N-ethyl-2-(5-fluoro-TH-indol-3-yl)-N-methylethan-1-amine, wherein olanzapine is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 15 minutes post to the administration of N-ethyl-2-(5-fluoro-1H-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 30 minutes post to the N-ethyl-2-(5-fluoro-1H-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 60 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 90 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 120 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 150 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat between about 15 minutes and about 150 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 180 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 210 minutes post to the N-ethyl-2-(5-fluoro-1H-indol-3-yl)-N-methylethan-1-amine. In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 240 minutes post to the N-ethyl-2-(5-fluoro-1H-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 270 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 300 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-TH-indol-3-yl)-N-methylethan-1-amine, wherein the quetiapine is administered to post-treat at least 330 minutes post to the N-ethyl-2-(5-fluoro-TH-indol-3-yl)-N-methylethan-1-amine. In some embodiments, the serotonin receptor modulator is quetiapine and the psychedelic is N-ethyl-2-(5-fluoro-1H-indol-3-yl)-N-methylethan-1-amine, wherein the quetiapine is administered to post-treat at least 360 minutes post to the N-ethyl-2-(5-fluoro-TH-indol-3-yl)-N-methylethan-1-amine. In some preferred embodiments, the serotonin receptor modulator is quetiapine and the psychedelic is N-ethyl-2-(5-fluoro-TH-indol-3-yl)-N-methylethan-1-amine, wherein quetiapine is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 15 minutes post to the administration of N-ethyl-2-(5-fluoro-1H-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 30 minutes post to the N-ethyl-2-(5-fluoro-1H-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 60 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-1H-indol-3-yl)-N-methylethan-1-amine, wherein the risperidone is administered to post-treat at least 90 minutes post to the N-ethyl-2-(5-fluoro-1H-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 120 minutes post to the N-ethyl-2-(5-fluoro-1H-indol-3-yl)-N-methylethan-1-amine. In some embodiments, the serotonin receptor modulator is risperidone and the psychedelic is N-ethyl-2-(5-fluoro-1H-indol-3-yl)-N-methylethan-1-amine, wherein the risperidone is administered to post-treat at least 150 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-1H-indol-3-yl)-N-methylethan-1-amine, wherein the risperidone is administered to post-treat between about 15 minutes and about 150 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 180 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 210 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-1H-indol-3-yl)-N-methylethan-1-amine, wherein the risperidone is administered to post-treat at least 240 minutes post to the N-ethyl-2-(5-fluoro-1H-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 270 minutes post to the N-ethyl-2-(5-fluoro-1H-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 300 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 330 minutes post to the N-ethyl-2-(5-fluoro-TH-indol-3-yl)-N-methylethan-1-amine. In some embodiments, the serotonin receptor modulator is risperidone and the psychedelic is N-ethyl-2-(5-fluoro-TH-indol-3-yl)-N-methylethan-1-amine, wherein the risperidone is administered to post-treat at least 360 minutes post to the N-ethyl-2-(5-fluoro-TH-indol-3-yl)-N-methylethan-1-amine. In some preferred embodiments, the serotonin receptor modulator is risperidone and the psychedelic is N-ethyl-2-(5-fluoro-1H-indol-3-yl)-N-methylethan-1-amine, wherein risperidone is administered to post-treat between about 60 minutes and about 180 minutes post to the administration of N-ethyl-2-(5-fluoro-1H-indol-3-yl)-N-methylethan-1-amine.

In some embodiments, the serotonin receptor modulator is eplivanserin and the psychedelic is 4-OH-DIPT hemi-glutarate, wherein the eplivanserin is administered to post-treat at least 15 minutes post to the administration of 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 30 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 60 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 90 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat between at least 30 minutes post and 360 minutes post to the release or administration of the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat between at least 60 minutes post and 360 minutes post to the release or administration the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat between at least 90 minutes and 240 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 120 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 150 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat between about 15 minutes and about 150 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 180 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 210 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 240 minutes post to the 4-OH-DIPT hemi-glutarate.

In some embodiments, the serotonin receptor modulator is eplivanserin and the psychedelic is 4-OH-DIPT hemi-glutarate, wherein the eplivanserin is administered to post-treat at least 270 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 300 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 330 minutes post to the 4-OH-DIPT hemi-glutarate. In some embodiments, the serotonin receptor modulator is eplivanserin and the psychedelic is 4-OH-DIPT hemi-glutarate, wherein the eplivanserin is administered to post-treat at least 360 minutes post to the 4-OH-DIPT hemi-glutarate.

In some preferred embodiments, the serotonin receptor modulator is eplivanserin and the psychedelic is 4-OH-DIPT hemi-glutarate, wherein eplivanserin is administered to post-treat between about 60 minutes and about 180 minutes post to the administration of 4-OH-DIPT hemi-glutarate.

In some embodiments, the serotonin receptor modulator is volinanserin and the psychedelic is 4-OH-DIPT hemi-glutarate, wherein the volinanserin is administered to post-treat at least 15 minutes post to the administration of 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 30 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 60 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat a subject between at least 15 minutes and 360 minutes post to the administration or release of 4-OH-DIPT hemi-glutarate. In some embodiments, the serotonin receptor modulator is volinanserin and the psychedelic is 4-OH-DIPT hemi-glutarate, wherein the volinanserin is administered to post-treat between at least 30 minutes and 360 minutes post to the administration or release of 4-OH-DIPT hemi-glutarate. In some embodiments, the serotonin receptor modulator is volinanserin and the psychedelic is 4-OH-DIPT hemi-glutarate, wherein the volinanserin is administered to post-treat between at least 60 minutes and 240 minutes post to the administration or release of 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 90 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 120 minutes post to the 4-OH-DIPT hemi-glutarate.

In some embodiments, the serotonin receptor modulator is volinanserin and the psychedelic is 4-OH-DIPT hemi-glutarate, wherein the volinanserin is administered to post-treat at least 150 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat between about 15 minutes and about 150 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 180 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 210 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 240 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 270 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 300 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 330 minutes post to the 4-OH-DIPT hemi-glutarate. In some embodiments, the serotonin receptor modulator is volinanserin and the psychedelic is 4-OH-DIPT hemi-glutarate, wherein the volinanserin is administered to post-treat at least 360 minutes post to the 4-OH-DIPT hemi-glutarate. In some preferred embodiments, the serotonin receptor modulator is volinanserin and the psychedelic is 4-OH-DIPT hemi-glutarate, wherein volinanserin is administered to post-treat between about 60 minutes and about 180 minutes post to the administration of 4-OH-DIPT hemi-glutarate.

In some embodiments, the serotonin receptor modulator is ketanserin and the psychedelic is 4-OH-DIPT hemi-glutarate, wherein the ketanserin is administered to post-treat at least 15 minutes post to the administration of 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 30 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 60 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat between at least 30 minutes and 360 minutes post to the administration or release 4-OH-DIPT hemi-glutarate. In some embodiments, the serotonin receptor modulator is ketanserin and the psychedelic is 4-OH-DIPT hemi-glutarate, wherein the ketanserin is administered to post-treat between at least 60 minutes and 240 minutes post to the administration or release of 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 90 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 120 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 150 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat between about 15 minutes and about 150 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 180 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 210 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 240 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 270 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 300 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 330 minutes post to the 4-OH-DIPT hemi-glutarate. In some embodiments, the serotonin receptor modulator is ketanserin and the psychedelic is 4-OH-DIPT hemi-glutarate, wherein the ketanserin is administered to post-treat at least 360 minutes post to the 4-OH-DIPT hemi-glutarate. In some preferred embodiments, the serotonin receptor modulator is ketanserin and the psychedelic is 4-OH-DIPT hemi-glutarate, wherein ketanserin is administered to post-treat between about 60 minutes and about 180 minutes post to the administration of 4-OH-DIPT hemi-glutarate.

In some embodiments, the serotonin receptor modulator is ritanserin and the psychedelic is 4-OH-DIPT hemi-glutarate, wherein the ritanserin is administered to post-treat at least 15 minutes post to the administration of 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 30 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 60 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat between at least 60 minutes and 240 minutes post to the administration or release of 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 90 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 120 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 150 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat between about 15 minutes and about 150 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 180 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 210 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 240 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 270 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 300 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 330 minutes post to the 4-OH-DIPT hemi-glutarate. In some embodiments, the serotonin receptor modulator is ritanserin and the psychedelic is 4-OH-DIPT hemi-glutarate, wherein the ritanserin is administered to post-treat at least 360 minutes post to the 4-OH-DIPT hemi-glutarate. In some preferred embodiments, the serotonin receptor modulator is ritanserin and the psychedelic is 4-OH-DIPT hemi-glutarate, wherein ritanserin is administered to post-treat between about 60 minutes and about 180 minutes post to the administration of 4-OH-DIPT hemi-glutarate.

In some embodiments, the serotonin receptor modulator is pimavanserin and the psychedelic is 4-OH-DIPT hemi-glutarate, wherein the pimavanserin is administered to post-treat at least 15 minutes post to the administration of 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 30 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 60 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat between at least 60 minutes and 240 minutes post to the administration or release of 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 90 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 120 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 150 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat between about 15 minutes and about 150 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 180 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 210 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 240 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 270 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 300 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 330 minutes post to the 4-OH-DIPT hemi-glutarate. In some embodiments, the serotonin receptor modulator is pimavanserin and the psychedelic is 4-OH-DIPT hemi-glutarate, wherein the pimavanserin is administered to post-treat at least 360 minutes post to the 4-OH-DIPT hemi-glutarate. In some preferred embodiments, the serotonin receptor modulator is pimavanserin and the psychedelic is 4-OH-DIPT hemi-glutarate, wherein pimavanserin is administered to post-treat between about 60 minutes and about 180 minutes post to the administration of 4-OH-DIPT hemi-glutarate.

In some embodiments, the serotonin receptor modulator is nelotanserin and the psychedelic is 4-OH-DIPT hemi-glutarate, wherein the nelotanserin is administered to post-treat at least 15 minutes post to the administration of 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 30 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 60 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat between at least 60 minutes and 240 minutes post to the administration or release of 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 90 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 120 minutes post to the 4-OH-DIPT hemi-glutarate.

In some embodiments, the serotonin receptor modulator is nelotanserin and the psychedelic is 4-OH-DIPT hemi-glutarate, wherein the nelotanserin is administered to post-treat at least 150 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat between about 15 minutes and about 150 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 180 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 210 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 240 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 270 minutes post to the 4-OH-DIPT hemi-glutarate.

In some embodiments, the serotonin receptor modulator is nelotanserin and the psychedelic is 4-OH-DIPT hemi-glutarate, wherein the nelotanserin is administered to post-treat at least 300 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 330 minutes post to the 4-OH-DIPT hemi-glutarate. In some embodiments, the serotonin receptor modulator is nelotanserin and the psychedelic is 4-OH-DIPT hemi-glutarate, wherein the nelotanserin is administered to post-treat at least 360 minutes post to the 4-OH-DIPT hemi-glutarate. In some preferred embodiments, the serotonin receptor modulator is nelotanserin and the psychedelic is 4-OH-DIPT hemi-glutarate, wherein nelotanserin is administered to post-treat between about 60 minutes and about 180 minutes post to the administration of 4-OH-DIPT hemi-glutarate.

In some embodiments, the serotonin receptor modulator is pruvanserin and the psychedelic is 4-OH-DIPT hemi-glutarate, wherein the pruvanserin is administered to post-treat at least 15 minutes post to the administration of 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 30 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 60 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat between at least 60 minutes and 240 minutes post to the administration or release of 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 90 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 120 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 150 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat between about 15 minutes and about 150 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 180 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 210 minutes post to the 4-OH-DIPT hemi-glutarate.

In some embodiments, the serotonin receptor modulator is pruvanserin and the psychedelic is 4-OH-DIPT hemi-glutarate, wherein the pruvanserin is administered to post-treat at least 240 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 270 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 300 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 330 minutes post to the 4-OH-DIPT hemi-glutarate. In some embodiments, the serotonin receptor modulator is pruvanserin and the psychedelic is 4-OH-DIPT hemi-glutarate, wherein the pruvanserin is administered to post-treat at least 360 minutes post to the 4-OH-DIPT hemi-glutarate. In some preferred embodiments, the serotonin receptor modulator is pruvanserin and the psychedelic is 4-OH-DIPT hemi-glutarate, wherein pruvanserin is administered to post-treat between about 60 minutes and about 180 minutes post to the administration of 4-OH-DIPT hemi-glutarate.

In some embodiments, the serotonin receptor modulator is flibanserin and the psychedelic is 4-OH-DIPT hemi-glutarate, wherein the flibanserin is administered to post-treat at least 15 minutes post to the administration of 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 30 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 60 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 90 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 120 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 150 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat between about 15 minutes and about 150 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 180 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 210 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 240 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 270 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 300 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 330 minutes post to the 4-OH-DIPT hemi-glutarate. In some embodiments, the serotonin receptor modulator is flibanserin and the psychedelic is 4-OH-DIPT hemi-glutarate, wherein the flibanserin is administered to post-treat at least 360 minutes post to the 4-OH-DIPT hemi-glutarate. In some preferred embodiments, the serotonin receptor modulator is flibanserin and the psychedelic is 4-OH-DIPT hemi-glutarate, wherein flibanserin is administered to post-treat between about 60 minutes and about 180 minutes post to the administration of 4-OH-DIPT hemi-glutarate.

In some embodiments, the serotonin receptor modulator is olanzapine and the psychedelic is 4-OH-DIPT hemi-glutarate, wherein the olanzapine is administered to post-treat at least 15 minutes post to the administration of 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 30 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 60 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 90 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 120 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 150 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat between about 15 minutes and about 150 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 180 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 210 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 240 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 270 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 300 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 330 minutes post to the 4-OH-DIPT hemi-glutarate. In some embodiments, the serotonin receptor modulator is olanzapine and the psychedelic is 4-OH-DIPT hemi-glutarate, wherein the olanzapine is administered to post-treat at least 360 minutes post to the 4-OH-DIPT hemi-glutarate. In some preferred embodiments, the serotonin receptor modulator is olanzapine and the psychedelic is 4-OH-DIPT hemi-glutarate, wherein olanzapine is administered to post-treat between about 60 minutes and about 180 minutes post to the administration of 4-OH-DIPT hemi-glutarate.

In some embodiments, the serotonin receptor modulator is quetiapine and the psychedelic is 4-OH-DIPT hemi-glutarate, wherein the quetiapine is administered to post-treat at least 15 minutes post to the administration of 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 30 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 60 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 90 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 120 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 150 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat between about 15 minutes and about 150 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 180 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 210 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 240 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 270 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 300 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 330 minutes post to the 4-OH-DIPT hemi-glutarate. In some embodiments, the serotonin receptor modulator is quetiapine and the psychedelic is 4-OH-DIPT hemi-glutarate, wherein the quetiapine is administered to post-treat at least 360 minutes post to the 4-OH-DIPT hemi-glutarate. In some preferred embodiments, the serotonin receptor modulator is quetiapine and the psychedelic is 4-OH-DIPT hemi-glutarate, wherein quetiapine is administered to post-treat between about 60 minutes and about 180 minutes post to the administration of 4-OH-DIPT hemi-glutarate.

In some embodiments, the serotonin receptor modulator is risperidone and the psychedelic is 4-OH-DIPT hemi-glutarate, wherein the risperidone is administered to post-treat at least 15 minutes post to the administration of 4-OH-DIPT hemi-glutarate. In some embodiments, the serotonin receptor modulator is risperidone and the psychedelic is 4-OH-DIPT hemi-glutarate, wherein the risperidone is administered to post-treat at least 30 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 60 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 90 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 120 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 150 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat between about 15 minutes and about 150 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 180 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 210 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 240 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 270 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 300 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 330 minutes post to the 4-OH-DIPT hemi-glutarate. In some embodiments, the serotonin receptor modulator is risperidone and the psychedelic is 4-OH-DIPT hemi-glutarate, wherein the risperidone is administered to post-treat at least 360 minutes post to the 4-OH-DIPT hemi-glutarate. In some preferred embodiments, the serotonin receptor modulator is risperidone and the psychedelic is 4-OH-DIPT hemi-glutarate, wherein risperidone is administered to post-treat between about 60 minutes and about 180 minutes post to the administration of 4-OH-DIPT hemi-glutarate.

In some embodiments, the serotonin receptor modulator is eplivanserin and the psychedelic is 5,6-dimethoxy-2-aminoindane, wherein the eplivanserin is administered to post-treat at least 15 minutes post to the administration of 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 30 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 60 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 90 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat between at least 30 minutes post and 360 minutes post to the release or administration of the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat between at least 60 minutes post and 360 minutes post to the release or administration the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat between at least 90 minutes and 240 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 120 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 150 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat between about 15 minutes and about 150 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 180 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 210 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 240 minutes post to the 5,6-dimethoxy-2-aminoindane.

In some embodiments, the serotonin receptor modulator is eplivanserin and the psychedelic is 5,6-dimethoxy-2-aminoindane, wherein the eplivanserin is administered to post-treat at least 270 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 300 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 330 minutes post to the 5,6-dimethoxy-2-aminoindane. In some embodiments, the serotonin receptor modulator is eplivanserin and the psychedelic is 5,6-dimethoxy-2-aminoindane, wherein the eplivanserin is administered to post-treat at least 360 minutes post to the 5,6-dimethoxy-2-aminoindane.

In some preferred embodiments, the serotonin receptor modulator is eplivanserin and the psychedelic is 5,6-dimethoxy-2-aminoindane, wherein eplivanserin is administered to post-treat between about 60 minutes and about 180 minutes post to the administration of 5,6-dimethoxy-2-aminoindane.

In some embodiments, the serotonin receptor modulator is volinanserin and the psychedelic is 5,6-dimethoxy-2-aminoindane, wherein the volinanserin is administered to post-treat at least 15 minutes post to the administration of 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 30 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 60 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat a subject between at least 15 minutes and 360 minutes post to the administration or release of 5,6-dimethoxy-2-aminoindane. In some embodiments, the serotonin receptor modulator is volinanserin and the psychedelic is 5,6-dimethoxy-2-aminoindane, wherein the volinanserin is administered to post-treat between at least 30 minutes and 360 minutes post to the administration or release of 5,6-dimethoxy-2-aminoindane. In some embodiments, the serotonin receptor modulator is volinanserin and the psychedelic is 5,6-dimethoxy-2-aminoindane, wherein the volinanserin is administered to post-treat between at least 60 minutes and 240 minutes post to the administration or release of 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 90 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 120 minutes post to the 5,6-dimethoxy-2-aminoindane.

In some embodiments, the serotonin receptor modulator is volinanserin and the psychedelic is 5,6-dimethoxy-2-aminoindane, wherein the volinanserin is administered to post-treat at least 150 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat between about 15 minutes and about 150 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 180 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 210 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 240 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 270 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 300 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 330 minutes post to the 5,6-dimethoxy-2-aminoindane. In some embodiments, the serotonin receptor modulator is volinanserin and the psychedelic is 5,6-dimethoxy-2-aminoindane, wherein the volinanserin is administered to post-treat at least 360 minutes post to the 5,6-dimethoxy-2-aminoindane. In some preferred embodiments, the serotonin receptor modulator is volinanserin and the psychedelic is 5,6-dimethoxy-2-aminoindane, wherein volinanserin is administered to post-treat between about 60 minutes and about 180 minutes post to the administration of 5,6-dimethoxy-2-aminoindane.

In some embodiments, the serotonin receptor modulator is ketanserin and the psychedelic is 5,6-dimethoxy-2-aminoindane, wherein the ketanserin is administered to post-treat at least 15 minutes post to the administration of 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 30 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 60 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat between at least 30 minutes and 360 minutes post to the administration or release 5,6-dimethoxy-2-aminoindane. In some embodiments, the serotonin receptor modulator is ketanserin and the psychedelic is 5,6-dimethoxy-2-aminoindane, wherein the ketanserin is administered to post-treat between at least 60 minutes and 240 minutes post to the administration or release of 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 90 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 120 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 150 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat between about 15 minutes and about 150 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 180 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 210 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 240 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 270 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 300 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 330 minutes post to the 5,6-dimethoxy-2-aminoindane. In some embodiments, the serotonin receptor modulator is ketanserin and the psychedelic is 5,6-dimethoxy-2-aminoindane, wherein the ketanserin is administered to post-treat at least 360 minutes post to the 5,6-dimethoxy-2-aminoindane. In some preferred embodiments, the serotonin receptor modulator is ketanserin and the psychedelic is 5,6-dimethoxy-2-aminoindane, wherein ketanserin is administered to post-treat between about 60 minutes and about 180 minutes post to the administration of 5,6-dimethoxy-2-aminoindane.

In some embodiments, the serotonin receptor modulator is ritanserin and the psychedelic is 5,6-dimethoxy-2-aminoindane, wherein the ritanserin is administered to post-treat at least 15 minutes post to the administration of 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 30 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 60 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat between at least 60 minutes and 240 minutes post to the administration or release of 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 90 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 120 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 150 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat between about 15 minutes and about 150 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 180 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 210 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 240 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 270 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 300 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 330 minutes post to the 5,6-dimethoxy-2-aminoindane. In some embodiments, the serotonin receptor modulator is ritanserin and the psychedelic is 5,6-dimethoxy-2-aminoindane, wherein the ritanserin is administered to post-treat at least 360 minutes post to the 5,6-dimethoxy-2-aminoindane. In some preferred embodiments, the serotonin receptor modulator is ritanserin and the psychedelic is 5,6-dimethoxy-2-aminoindane, wherein ritanserin is administered to post-treat between about 60 minutes and about 180 minutes post to the administration of 5,6-dimethoxy-2-aminoindane.

In some embodiments, the serotonin receptor modulator is pimavanserin and the psychedelic is 5,6-dimethoxy-2-aminoindane, wherein the pimavanserin is administered to post-treat at least 15 minutes post to the administration of 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 30 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 60 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat between at least 60 minutes and 240 minutes post to the administration or release of 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 90 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 120 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 150 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat between about 15 minutes and about 150 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 180 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 210 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 240 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 270 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 300 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 330 minutes post to the 5,6-dimethoxy-2-aminoindane. In some embodiments, the serotonin receptor modulator is pimavanserin and the psychedelic is 5,6-dimethoxy-2-aminoindane, wherein the pimavanserin is administered to post-treat at least 360 minutes post to the 5,6-dimethoxy-2-aminoindane. In some preferred embodiments, the serotonin receptor modulator is pimavanserin and the psychedelic is 5,6-dimethoxy-2-aminoindane, wherein pimavanserin is administered to post-treat between about 60 minutes and about 180 minutes post to the administration of 5,6-dimethoxy-2-aminoindane.

In some embodiments, the serotonin receptor modulator is nelotanserin and the psychedelic is 5,6-dimethoxy-2-aminoindane, wherein the nelotanserin is administered to post-treat at least 15 minutes post to the administration of 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 30 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 60 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat between at least 60 minutes and 240 minutes post to the administration or release of 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 90 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 120 minutes post to the 5,6-dimethoxy-2-aminoindane.

In some embodiments, the serotonin receptor modulator is nelotanserin and the psychedelic is 5,6-dimethoxy-2-aminoindane, wherein the nelotanserin is administered to post-treat at least 150 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat between about 15 minutes and about 150 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 180 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 210 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 240 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 270 minutes post to the 5,6-dimethoxy-2-aminoindane.

In some embodiments, the serotonin receptor modulator is nelotanserin and the psychedelic is 5,6-dimethoxy-2-aminoindane, wherein the nelotanserin is administered to post-treat at least 300 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 330 minutes post to the 5,6-dimethoxy-2-aminoindane. In some embodiments, the serotonin receptor modulator is nelotanserin and the psychedelic is 5,6-dimethoxy-2-aminoindane, wherein the nelotanserin is administered to post-treat at least 360 minutes post to the 5,6-dimethoxy-2-aminoindane. In some preferred embodiments, the serotonin receptor modulator is nelotanserin and the psychedelic is 5,6-dimethoxy-2-aminoindane, wherein nelotanserin is administered to post-treat between about 60 minutes and about 180 minutes post to the administration of 5,6-dimethoxy-2-aminoindane.

In some embodiments, the serotonin receptor modulator is pruvanserin and the psychedelic is 5,6-dimethoxy-2-aminoindane, wherein the pruvanserin is administered to post-treat at least 15 minutes post to the administration of 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 30 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 60 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat between at least 60 minutes and 240 minutes post to the administration or release of 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 90 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 120 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 150 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat between about 15 minutes and about 150 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 180 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 210 minutes post to the 5,6-dimethoxy-2-aminoindane.

In some embodiments, the serotonin receptor modulator is pruvanserin and the psychedelic is 5,6-dimethoxy-2-aminoindane, wherein the pruvanserin is administered to post-treat at least 240 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 270 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 300 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 330 minutes post to the 5,6-dimethoxy-2-aminoindane. In some embodiments, the serotonin receptor modulator is pruvanserin and the psychedelic is 5,6-dimethoxy-2-aminoindane, wherein the pruvanserin is administered to post-treat at least 360 minutes post to the 5,6-dimethoxy-2-aminoindane. In some preferred embodiments, the serotonin receptor modulator is pruvanserin and the psychedelic is 5,6-dimethoxy-2-aminoindane, wherein pruvanserin is administered to post-treat between about 60 minutes and about 180 minutes post to the administration of 5,6-dimethoxy-2-aminoindane.

In some embodiments, the serotonin receptor modulator is flibanserin and the psychedelic is 5,6-dimethoxy-2-aminoindane, wherein the flibanserin is administered to post-treat at least 15 minutes post to the administration of 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 30 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 60 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 90 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 120 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 150 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat between about 15 minutes and about 150 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 180 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 210 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 240 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 270 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 300 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 330 minutes post to the 5,6-dimethoxy-2-aminoindane. In some embodiments, the serotonin receptor modulator is flibanserin and the psychedelic is 5,6-dimethoxy-2-aminoindane, wherein the flibanserin is administered to post-treat at least 360 minutes post to the 5,6-dimethoxy-2-aminoindane. In some preferred embodiments, the serotonin receptor modulator is flibanserin and the psychedelic is 5,6-dimethoxy-2-aminoindane, wherein flibanserin is administered to post-treat between about 60 minutes and about 180 minutes post to the administration of 5,6-dimethoxy-2-aminoindane.

In some embodiments, the serotonin receptor modulator is olanzapine and the psychedelic is 5,6-dimethoxy-2-aminoindane, wherein the olanzapine is administered to post-treat at least 15 minutes post to the administration of 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 30 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 60 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 90 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 120 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 150 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat between about 15 minutes and about 150 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 180 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 210 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 240 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 270 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 300 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 330 minutes post to the 5,6-dimethoxy-2-aminoindane. In some embodiments, the serotonin receptor modulator is olanzapine and the psychedelic is 5,6-dimethoxy-2-aminoindane, wherein the olanzapine is administered to post-treat at least 360 minutes post to the 5,6-dimethoxy-2-aminoindane. In some preferred embodiments, the serotonin receptor modulator is olanzapine and the psychedelic is 5,6-dimethoxy-2-aminoindane, wherein olanzapine is administered to post-treat between about 60 minutes and about 180 minutes post to the administration of 5,6-dimethoxy-2-aminoindane.

In some embodiments, the serotonin receptor modulator is quetiapine and the psychedelic is 5,6-dimethoxy-2-aminoindane, wherein the quetiapine is administered to post-treat at least 15 minutes post to the administration of 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 30 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 60 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 90 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 120 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 150 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat between about 15 minutes and about 150 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 180 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 210 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 240 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 270 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 300 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 330 minutes post to the 5,6-dimethoxy-2-aminoindane. In some embodiments, the serotonin receptor modulator is quetiapine and the psychedelic is 5,6-dimethoxy-2-aminoindane, wherein the quetiapine is administered to post-treat at least 360 minutes post to the 5,6-dimethoxy-2-aminoindane. In some preferred embodiments, the serotonin receptor modulator is quetiapine and the psychedelic is 5,6-dimethoxy-2-aminoindane, wherein quetiapine is administered to post-treat between about 60 minutes and about 180 minutes post to the administration of 5,6-dimethoxy-2-aminoindane.

In some embodiments, the serotonin receptor modulator is risperidone and the psychedelic is 5,6-dimethoxy-2-aminoindane, wherein the risperidone is administered to post-treat at least 15 minutes post to the administration of 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 30 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 60 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 90 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 120 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 150 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat between about 15 minutes and about 150 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 180 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 210 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 240 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 270 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 300 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 330 minutes post to the 5,6-dimethoxy-2-aminoindane. In some embodiments, the serotonin receptor modulator is risperidone and the psychedelic is 5,6-dimethoxy-2-aminoindane, wherein the risperidone is administered to post-treat at least 360 minutes post to the 5,6-dimethoxy-2-aminoindane. In some preferred embodiments, the serotonin receptor modulator is risperidone and the psychedelic is 5,6-dimethoxy-2-aminoindane, wherein risperidone is administered to post-treat between about 60 minutes and about 180 minutes post to the administration of 5,6-dimethoxy-2-aminoindane.

In some embodiments, the serotonin receptor modulator is eplivanserin and the psychedelic is 5-methoxy-2-aminoindane, wherein the eplivanserin is administered to post-treat at least 15 minutes post to the administration of 5-methoxy-2-aminoindane. In some embodiments, the serotonin receptor modulator is eplivanserin and the psychedelic is 5-methoxy-2-aminoindane, wherein the eplivanserin is administered to post-treat at least 30 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 60 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 90 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat between at least 30 minutes post and 360 minutes post to the release or administration of the 5-methoxy-2-aminoindane. In some embodiments, the serotonin receptor modulator is eplivanserin and the psychedelic is 5-methoxy-2-aminoindane, wherein the eplivanserin is administered to post-treat between at least 60 minutes post and 360 minutes post to the release or administration the 5-methoxy-2-aminoindane. In some embodiments, the serotonin receptor modulator is eplivanserin and the psychedelic is 5-methoxy-2-aminoindane, wherein the eplivanserin is administered to post-treat between at least 90 minutes and 240 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 120 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 150 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat between about 15 minutes and about 150 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 180 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 210 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 240 minutes post to the 5-methoxy-2-aminoindane.

In some embodiments, the serotonin receptor modulator is eplivanserin and the psychedelic is 5-methoxy-2-aminoindane, wherein the eplivanserin is administered to post-treat at least 270 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 300 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 330 minutes post to the 5-methoxy-2-aminoindane. In some embodiments, the serotonin receptor modulator is eplivanserin and the psychedelic is 5-methoxy-2-aminoindane, wherein the eplivanserin is administered to post-treat at least 360 minutes post to the 5-methoxy-2-aminoindane.

In some preferred embodiments, the serotonin receptor modulator is eplivanserin and the psychedelic is 5-methoxy-2-aminoindane, wherein eplivanserin is administered to post-treat between about 60 minutes and about 180 minutes post to the administration of 5-methoxy-2-aminoindane.

In some embodiments, the serotonin receptor modulator is volinanserin and the psychedelic is 5-methoxy-2-aminoindane, wherein the volinanserin is administered to post-treat at least 15 minutes post to the administration of 5-methoxy-2-aminoindane. In some embodiments, the serotonin receptor modulator is volinanserin and the psychedelic is 5-methoxy-2-aminoindane, wherein the volinanserin is administered to post-treat at least 30 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 60 minutes post to the 5-methoxy-2-aminoindane. In some embodiments, the serotonin receptor modulator is volinanserin and the psychedelic is 5-methoxy-2-aminoindane, wherein the volinanserin is administered to post-treat a subject between at least 15 minutes and 360 minutes post to the administration or release of 5-methoxy-2-aminoindane. In some embodiments, the serotonin receptor modulator is volinanserin and the psychedelic is 5-methoxy-2-aminoindane, wherein the volinanserin is administered to post-treat between at least 30 minutes and 360 minutes post to the administration or release of 5-methoxy-2-aminoindane. In some embodiments, the serotonin receptor modulator is volinanserin and the psychedelic is 5-methoxy-2-aminoindane, wherein the volinanserin is administered to post-treat between at least 60 minutes and 240 minutes post to the administration or release of 5-methoxy-2-aminoindane. In some embodiments, the serotonin receptor modulator is volinanserin and the psychedelic is 5-methoxy-2-aminoindane, wherein the volinanserin is administered to post-treat at least 90 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 120 minutes post to the 5-methoxy-2-aminoindane.

In some embodiments, the serotonin receptor modulator is volinanserin and the psychedelic is 5-methoxy-2-aminoindane, wherein the volinanserin is administered to post-treat at least 150 minutes post to the 5-methoxy-2-aminoindane. In some embodiments, the serotonin receptor modulator is volinanserin and the psychedelic is 5-methoxy-2-aminoindane, wherein the volinanserin is administered to post-treat between about 15 minutes and about 150 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 180 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 210 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 240 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 270 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 300 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 330 minutes post to the 5-methoxy-2-aminoindane. In some embodiments, the serotonin receptor modulator is volinanserin and the psychedelic is 5-methoxy-2-aminoindane, wherein the volinanserin is administered to post-treat at least 360 minutes post to the 5-methoxy-2-aminoindane. In some preferred embodiments, the serotonin receptor modulator is volinanserin and the psychedelic is 5-methoxy-2-aminoindane, wherein volinanserin is administered to post-treat between about 60 minutes and about 180 minutes post to the administration of 5-methoxy-2-aminoindane.

In some embodiments, the serotonin receptor modulator is ketanserin and the psychedelic is 5-methoxy-2-aminoindane, wherein the ketanserin is administered to post-treat at least 15 minutes post to the administration of 5-methoxy-2-aminoindane. In some embodiments, the serotonin receptor modulator is ketanserin and the psychedelic is 5-methoxy-2-aminoindane, wherein the ketanserin is administered to post-treat at least 30 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 60 minutes post to the 5-methoxy-2-aminoindane. In some embodiments, the serotonin receptor modulator is ketanserin and the psychedelic is 5-methoxy-2-aminoindane, wherein the ketanserin is administered to post-treat between at least 30 minutes and 360 minutes post to the administration or release 5-methoxy-2-aminoindane. In some embodiments, the serotonin receptor modulator is ketanserin and the psychedelic is 5-methoxy-2-aminoindane, wherein the ketanserin is administered to post-treat between at least 60 minutes and 240 minutes post to the administration or release of 5-methoxy-2-aminoindane. In some embodiments, the serotonin receptor modulator is ketanserin and the psychedelic is 5-methoxy-2-aminoindane, wherein the ketanserin is administered to post-treat at least 90 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 120 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 150 minutes post to the 5-methoxy-2-aminoindane. In some embodiments, the serotonin receptor modulator is ketanserin and the psychedelic is 5-methoxy-2-aminoindane, wherein the ketanserin is administered to post-treat between about 15 minutes and about 150 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 180 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 210 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 240 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 270 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 300 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 330 minutes post to the 5-methoxy-2-aminoindane. In some embodiments, the serotonin receptor modulator is ketanserin and the psychedelic is 5-methoxy-2-aminoindane, wherein the ketanserin is administered to post-treat at least 360 minutes post to the 5-methoxy-2-aminoindane. In some preferred embodiments, the serotonin receptor modulator is ketanserin and the psychedelic is 5-methoxy-2-aminoindane, wherein ketanserin is administered to post-treat between about 60 minutes and about 180 minutes post to the administration of 5-methoxy-2-aminoindane.

In some embodiments, the serotonin receptor modulator is ritanserin and the psychedelic is 5-methoxy-2-aminoindane, wherein the ritanserin is administered to post-treat at least 15 minutes post to the administration of 5-methoxy-2-aminoindane. In some embodiments, the serotonin receptor modulator is ritanserin and the psychedelic is 5-methoxy-2-aminoindane, wherein the ritanserin is administered to post-treat at least 30 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 60 minutes post to the 5-methoxy-2-aminoindane. In some embodiments, the serotonin receptor modulator is ritanserin and the psychedelic is 5-methoxy-2-aminoindane, wherein the ritanserin is administered to post-treat between at least 60 minutes and 240 minutes post to the administration or release of 5-methoxy-2-aminoindane. In some embodiments, the serotonin receptor modulator is ritanserin and the psychedelic is 5-methoxy-2-aminoindane, wherein the ritanserin is administered to post-treat at least 90 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 120 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 150 minutes post to the 5-methoxy-2-aminoindane. In some embodiments, the serotonin receptor modulator is ritanserin and the psychedelic is 5-methoxy-2-aminoindane, wherein the ritanserin is administered to post-treat between about 15 minutes and about 150 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 180 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 210 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 240 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 270 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 300 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 330 minutes post to the 5-methoxy-2-aminoindane. In some embodiments, the serotonin receptor modulator is ritanserin and the psychedelic is 5-methoxy-2-aminoindane, wherein the ritanserin is administered to post-treat at least 360 minutes post to the 5-methoxy-2-aminoindane. In some preferred embodiments, the serotonin receptor modulator is ritanserin and the psychedelic is 5-methoxy-2-aminoindane, wherein ritanserin is administered to post-treat between about 60 minutes and about 180 minutes post to the administration of 5-methoxy-2-aminoindane.

In some embodiments, the serotonin receptor modulator is pimavanserin and the psychedelic is 5-methoxy-2-aminoindane, wherein the pimavanserin is administered to post-treat at least 15 minutes post to the administration of 5-methoxy-2-aminoindane. In some embodiments, the serotonin receptor modulator is pimavanserin and the psychedelic is 5-methoxy-2-aminoindane, wherein the pimavanserin is administered to post-treat at least 30 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 60 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat between at least 60 minutes and 240 minutes post to the administration or release of 5-methoxy-2-aminoindane. In some embodiments, the serotonin receptor modulator is pimavanserin and the psychedelic is 5-methoxy-2-aminoindane, wherein the pimavanserin is administered to post-treat at least 90 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 120 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 150 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat between about 15 minutes and about 150 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 180 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 210 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 240 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 270 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 300 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 330 minutes post to the 5-methoxy-2-aminoindane. In some embodiments, the serotonin receptor modulator is pimavanserin and the psychedelic is 5-methoxy-2-aminoindane, wherein the pimavanserin is administered to post-treat at least 360 minutes post to the 5-methoxy-2-aminoindane. In some preferred embodiments, the serotonin receptor modulator is pimavanserin and the psychedelic is 5-methoxy-2-aminoindane, wherein pimavanserin is administered to post-treat between about 60 minutes and about 180 minutes post to the administration of 5-methoxy-2-aminoindane.

In some embodiments, the serotonin receptor modulator is nelotanserin and the psychedelic is 5-methoxy-2-aminoindane, wherein the nelotanserin is administered to post-treat at least 15 minutes post to the administration of 5-methoxy-2-aminoindane. In some embodiments, the serotonin receptor modulator is nelotanserin and the psychedelic is 5-methoxy-2-aminoindane, wherein the nelotanserin is administered to post-treat at least 30 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 60 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat between at least 60 minutes and 240 minutes post to the administration or release of 5-methoxy-2-aminoindane. In some embodiments, the serotonin receptor modulator is nelotanserin and the psychedelic is 5-methoxy-2-aminoindane, wherein the nelotanserin is administered to post-treat at least 90 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 120 minutes post to the 5-methoxy-2-aminoindane.

In some embodiments, the serotonin receptor modulator is nelotanserin and the psychedelic is 5-methoxy-2-aminoindane, wherein the nelotanserin is administered to post-treat at least 150 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat between about 15 minutes and about 150 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 180 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 210 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 240 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 270 minutes post to the 5-methoxy-2-aminoindane.

In some embodiments, the serotonin receptor modulator is nelotanserin and the psychedelic is 5-methoxy-2-aminoindane, wherein the nelotanserin is administered to post-treat at least 300 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 330 minutes post to the 5-methoxy-2-aminoindane. In some embodiments, the serotonin receptor modulator is nelotanserin and the psychedelic is 5-methoxy-2-aminoindane, wherein the nelotanserin is administered to post-treat at least 360 minutes post to the 5-methoxy-2-aminoindane. In some preferred embodiments, the serotonin receptor modulator is nelotanserin and the psychedelic is 5-methoxy-2-aminoindane, wherein nelotanserin is administered to post-treat between about 60 minutes and about 180 minutes post to the administration of 5-methoxy-2-aminoindane.

In some embodiments, the serotonin receptor modulator is pruvanserin and the psychedelic is 5-methoxy-2-aminoindane, wherein the pruvanserin is administered to post-treat at least 15 minutes post to the administration of 5-methoxy-2-aminoindane. In some embodiments, the serotonin receptor modulator is pruvanserin and the psychedelic is 5-methoxy-2-aminoindane, wherein the pruvanserin is administered to post-treat at least 30 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 60 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat between at least 60 minutes and 240 minutes post to the administration or release of 5-methoxy-2-aminoindane. In some embodiments, the serotonin receptor modulator is pruvanserin and the psychedelic is 5-methoxy-2-aminoindane, wherein the pruvanserin is administered to post-treat at least 90 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 120 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 150 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat between about 15 minutes and about 150 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 180 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 210 minutes post to the 5-methoxy-2-aminoindane.

In some embodiments, the serotonin receptor modulator is pruvanserin and the psychedelic is 5-methoxy-2-aminoindane, wherein the pruvanserin is administered to post-treat at least 240 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 270 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 300 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 330 minutes post to the 5-methoxy-2-aminoindane. In some embodiments, the serotonin receptor modulator is pruvanserin and the psychedelic is 5-methoxy-2-aminoindane, wherein the pruvanserin is administered to post-treat at least 360 minutes post to the 5-methoxy-2-aminoindane. In some preferred embodiments, the serotonin receptor modulator is pruvanserin and the psychedelic is 5-methoxy-2-aminoindane, wherein pruvanserin is administered to post-treat between about 60 minutes and about 180 minutes post to the administration of 5-methoxy-2-aminoindane.

In some embodiments, the serotonin receptor modulator is flibanserin and the psychedelic is 5-methoxy-2-aminoindane, wherein the flibanserin is administered to post-treat at least 15 minutes post to the administration of 5-methoxy-2-aminoindane. In some embodiments, the serotonin receptor modulator is flibanserin and the psychedelic is 5-methoxy-2-aminoindane, wherein the flibanserin is administered to post-treat at least 30 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 60 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 90 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 120 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 150 minutes post to the 5-methoxy-2-aminoindane. In some embodiments, the serotonin receptor modulator is flibanserin and the psychedelic is 5-methoxy-2-aminoindane, wherein the flibanserin is administered to post-treat between about 15 minutes and about 150 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 180 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 210 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 240 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 270 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 300 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 330 minutes post to the 5-methoxy-2-aminoindane. In some embodiments, the serotonin receptor modulator is flibanserin and the psychedelic is 5-methoxy-2-aminoindane, wherein the flibanserin is administered to post-treat at least 360 minutes post to the 5-methoxy-2-aminoindane. In some preferred embodiments, the serotonin receptor modulator is flibanserin and the psychedelic is 5-methoxy-2-aminoindane, wherein flibanserin is administered to post-treat between about 60 minutes and about 180 minutes post to the administration of 5-methoxy-2-aminoindane.

In some embodiments, the serotonin receptor modulator is olanzapine and the psychedelic is 5-methoxy-2-aminoindane, wherein the olanzapine is administered to post-treat at least 15 minutes post to the administration of 5-methoxy-2-aminoindane. In some embodiments, the serotonin receptor modulator is olanzapine and the psychedelic is 5-methoxy-2-aminoindane, wherein the olanzapine is administered to post-treat at least 30 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 60 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 90 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 120 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 150 minutes post to the 5-methoxy-2-aminoindane. In some embodiments, the serotonin receptor modulator is olanzapine and the psychedelic is 5-methoxy-2-aminoindane, wherein the olanzapine is administered to post-treat between about 15 minutes and about 150 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 180 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 210 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 240 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 270 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 300 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 330 minutes post to the 5-methoxy-2-aminoindane. In some embodiments, the serotonin receptor modulator is olanzapine and the psychedelic is 5-methoxy-2-aminoindane, wherein the olanzapine is administered to post-treat at least 360 minutes post to the 5-methoxy-2-aminoindane. In some preferred embodiments, the serotonin receptor modulator is olanzapine and the psychedelic is 5-methoxy-2-aminoindane, wherein olanzapine is administered to post-treat between about 60 minutes and about 180 minutes post to the administration of 5-methoxy-2-aminoindane.

In some embodiments, the serotonin receptor modulator is quetiapine and the psychedelic is 5-methoxy-2-aminoindane, wherein the quetiapine is administered to post-treat at least 15 minutes post to the administration of 5-methoxy-2-aminoindane. In some embodiments, the serotonin receptor modulator is quetiapine and the psychedelic is 5-methoxy-2-aminoindane, wherein the quetiapine is administered to post-treat at least 30 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 60 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 90 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 120 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 150 minutes post to the 5-methoxy-2-aminoindane. In some embodiments, the serotonin receptor modulator is quetiapine and the psychedelic is 5-methoxy-2-aminoindane, wherein the quetiapine is administered to post-treat between about 15 minutes and about 150 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 180 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 210 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 240 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 270 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 300 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 330 minutes post to the 5-methoxy-2-aminoindane. In some embodiments, the serotonin receptor modulator is quetiapine and the psychedelic is 5-methoxy-2-aminoindane, wherein the quetiapine is administered to post-treat at least 360 minutes post to the 5-methoxy-2-aminoindane. In some preferred embodiments, the serotonin receptor modulator is quetiapine and the psychedelic is 5-methoxy-2-aminoindane, wherein quetiapine is administered to post-treat between about 60 minutes and about 180 minutes post to the administration of 5-methoxy-2-aminoindane.

In some embodiments, the serotonin receptor modulator is risperidone and the psychedelic is 5-methoxy-2-aminoindane, wherein the risperidone is administered to post-treat at least 15 minutes post to the administration of 5-methoxy-2-aminoindane. In some embodiments, the serotonin receptor modulator is risperidone and the psychedelic is 5-methoxy-2-aminoindane, wherein the risperidone is administered to post-treat at least 30 minutes post to the 5-methoxy-2-aminoindane. In some embodiments, the serotonin receptor modulator is risperidone and the psychedelic is 5-methoxy-2-aminoindane, wherein the risperidone is administered to post-treat at least 60 minutes post to the 5-methoxy-2-aminoindane. In some embodiments, the serotonin receptor modulator is risperidone and the psychedelic is 5-methoxy-2-aminoindane, wherein the risperidone is administered to post-treat at least 90 minutes post to the 5-methoxy-2-aminoindane. In some embodiments, the serotonin receptor modulator is risperidone and the psychedelic is 5-methoxy-2-aminoindane, wherein the risperidone is administered to post-treat at least 120 minutes post to the 5-methoxy-2-aminoindane. In some embodiments, the serotonin receptor modulator is risperidone and the psychedelic is 5-methoxy-2-aminoindane, wherein the risperidone is administered to post-treat at least 150 minutes post to the 5-methoxy-2-aminoindane. In some embodiments, the serotonin receptor modulator is risperidone and the psychedelic is 5-methoxy-2-aminoindane, wherein the risperidone is administered to post-treat between about 15 minutes and about 150 minutes post to the 5-methoxy-2-aminoindane. In some embodiments, the serotonin receptor modulator is risperidone and the psychedelic is 5-methoxy-2-aminoindane, wherein the risperidone is administered to post-treat at least 180 minutes post to the 5-methoxy-2-aminoindane. In some embodiments, the serotonin receptor modulator is risperidone and the psychedelic is 5-methoxy-2-aminoindane, wherein the risperidone is administered to post-treat at least 210 minutes post to the 5-methoxy-2-aminoindane. In some embodiments, the serotonin receptor modulator is risperidone and the psychedelic is 5-methoxy-2-aminoindane, wherein the risperidone is administered to post-treat at least 240 minutes post to the 5-methoxy-2-aminoindane. In some embodiments, the serotonin receptor modulator is risperidone and the psychedelic is 5-methoxy-2-aminoindane, wherein the risperidone is administered to post-treat at least 270 minutes post to the 5-methoxy-2-aminoindane. In some embodiments, the serotonin receptor modulator is risperidone and the psychedelic is 5-methoxy-2-aminoindane, wherein the risperidone is administered to post-treat at least 300 minutes post to the 5-methoxy-2-aminoindane. In some embodiments, the serotonin receptor modulator is risperidone and the psychedelic is 5-methoxy-2-aminoindane, wherein the risperidone is administered to post-treat at least 330 minutes post to the 5-methoxy-2-aminoindane. In some embodiments, the serotonin receptor modulator is risperidone and the psychedelic is 5-methoxy-2-aminoindane, wherein the risperidone is administered to post-treat at least 360 minutes post to the 5-methoxy-2-aminoindane. In some preferred embodiments, the serotonin receptor modulator is risperidone and the psychedelic is 5-methoxy-2-aminoindane, wherein risperidone is administered to post-treat between about 60 minutes and about 180 minutes post to the administration of 5-methoxy-2-aminoindane.

In some embodiments, the serotonin receptor modulator is eplivanserin and the psychedelic is 2-Br-LSD, wherein the eplivanserin is administered to post-treat at least 15 minutes post to the administration of 2-Br-LSD. In some embodiments, the serotonin receptor modulator is eplivanserin and the psychedelic is 2-Br-LSD, wherein the eplivanserin is administered to post-treat at least 30 minutes post to the 2-Br-LSD. In some embodiments, the serotonin receptor modulator is eplivanserin and the psychedelic is 2-Br-LSD, wherein the eplivanserin is administered to post-treat at least 60 minutes post to the 2-Br-LSD. In some embodiments, the serotonin receptor modulator is eplivanserin and the psychedelic is 2-Br-LSD, wherein the eplivanserin is administered to post-treat at least 90 minutes post to the 2-Br-LSD. In some embodiments, the serotonin receptor modulator is eplivanserin and the psychedelic is 2-Br-LSD, wherein the eplivanserin is administered to post-treat between at least 30 minutes post and 360 minutes post to the release or administration of the 2-Br-LSD. In some embodiments, the serotonin receptor modulator is eplivanserin and the psychedelic is 2-Br-LSD, wherein the eplivanserin is administered to post-treat between at least 60 minutes post and 360 minutes post to the release or administration the 2-Br-LSD. In some embodiments, the serotonin receptor modulator is eplivanserin and the psychedelic is 2-Br-LSD, wherein the eplivanserin is administered to post-treat between at least 90 minutes and 240 minutes post to the 2-Br-LSD. In some embodiments, the serotonin receptor modulator is eplivanserin and the psychedelic is 2-Br-LSD, wherein the eplivanserin is administered to post-treat at least 120 minutes post to the 2-Br-LSD. In some embodiments, the serotonin receptor modulator is eplivanserin and the psychedelic is 2-Br-LSD, wherein the eplivanserin is administered to post-treat at least 150 minutes post to the 2-Br-LSD. In some embodiments, the serotonin receptor modulator is eplivanserin and the psychedelic is 2-Br-LSD, wherein the eplivanserin is administered to post-treat between about 15 minutes and about 150 minutes post to the 2-Br-LSD. In some embodiments, the serotonin receptor modulator is eplivanserin and the psychedelic is 2-Br-LSD, wherein the eplivanserin is administered to post-treat at least 180 minutes post to the 2-Br-LSD. In some embodiments, the serotonin receptor modulator is eplivanserin and the psychedelic is 2-Br-LSD, wherein the eplivanserin is administered to post-treat at least 210 minutes post to the 2-Br-LSD. In some embodiments, the serotonin receptor modulator is eplivanserin and the psychedelic is 2-Br-LSD, wherein the eplivanserin is administered to post-treat at least 240 minutes post to the 2-Br-LSD.

In some embodiments, the serotonin receptor modulator is eplivanserin and the psychedelic is 2-Br-LSD, wherein the eplivanserin is administered to post-treat at least 270 minutes post to the 2-Br-LSD. In some embodiments, the serotonin receptor modulator is eplivanserin and the psychedelic is 2-Br-LSD, wherein the eplivanserin is administered to post-treat at least 300 minutes post to the 2-Br-LSD. In some embodiments, the serotonin receptor modulator is eplivanserin and the psychedelic is 2-Br-LSD, wherein the eplivanserin is administered to post-treat at least 330 minutes post to the 2-Br-LSD. In some embodiments, the serotonin receptor modulator is eplivanserin and the psychedelic is 2-Br-LSD, wherein the eplivanserin is administered to post-treat at least 360 minutes post to the 2-Br-LSD.

In some preferred embodiments, the serotonin receptor modulator is eplivanserin and the psychedelic is 2-Br-LSD, wherein eplivanserin is administered to post-treat between about 60 minutes and about 180 minutes post to the administration of 2-Br-LSD.

In some embodiments, the serotonin receptor modulator is volinanserin and the psychedelic is 2-Br-LSD, wherein the volinanserin is administered to post-treat at least 15 minutes post to the administration of 2-Br-LSD. In some embodiments, the serotonin receptor modulator is volinanserin and the psychedelic is 2-Br-LSD, wherein the volinanserin is administered to post-treat at least 30 minutes post to the 2-Br-LSD. In some embodiments, the serotonin receptor modulator is volinanserin and the psychedelic is 2-Br-LSD, wherein the volinanserin is administered to post-treat at least 60 minutes post to the 2-Br-LSD. In some embodiments, the serotonin receptor modulator is volinanserin and the psychedelic is 2-Br-LSD, wherein the volinanserin is administered to post-treat a subject between at least 15 minutes and 360 minutes post to the administration or release of 2-Br-LSD. In some embodiments, the serotonin receptor modulator is volinanserin and the psychedelic is 2-Br-LSD, wherein the volinanserin is administered to post-treat between at least 30 minutes and 360 minutes post to the administration or release of 2-Br-LSD. In some embodiments, the serotonin receptor modulator is volinanserin and the psychedelic is 2-Br-LSD, wherein the volinanserin is administered to post-treat between at least 60 minutes and 240 minutes post to the administration or release of 2-Br-LSD. In some embodiments, the serotonin receptor modulator is volinanserin and the psychedelic is 2-Br-LSD, wherein the volinanserin is administered to post-treat at least 90 minutes post to the 2-Br-LSD. In some embodiments, the serotonin receptor modulator is volinanserin and the psychedelic is 2-Br-LSD, wherein the volinanserin is administered to post-treat at least 120 minutes post to the 2-Br-LSD.

In some embodiments, the serotonin receptor modulator is volinanserin and the psychedelic is 2-Br-LSD, wherein the volinanserin is administered to post-treat at least 150 minutes post to the 2-Br-LSD. In some embodiments, the serotonin receptor modulator is volinanserin and the psychedelic is 2-Br-LSD, wherein the volinanserin is administered to post-treat between about 15 minutes and about 150 minutes post to the 2-Br-LSD. In some embodiments, the serotonin receptor modulator is volinanserin and the psychedelic is 2-Br-LSD, wherein the volinanserin is administered to post-treat at least 180 minutes post to the 2-Br-LSD. In some embodiments, the serotonin receptor modulator is volinanserin and the psychedelic is 2-Br-LSD, wherein the volinanserin is administered to post-treat at least 210 minutes post to the 2-Br-LSD. In some embodiments, the serotonin receptor modulator is volinanserin and the psychedelic is 2-Br-LSD, wherein the volinanserin is administered to post-treat at least 240 minutes post to the 2-Br-LSD. In some embodiments, the serotonin receptor modulator is volinanserin and the psychedelic is 2-Br-LSD, wherein the volinanserin is administered to post-treat at least 270 minutes post to the 2-Br-LSD. In some embodiments, the serotonin receptor modulator is volinanserin and the psychedelic is 2-Br-LSD, wherein the volinanserin is administered to post-treat at least 300 minutes post to the 2-Br-LSD. In some embodiments, the serotonin receptor modulator is volinanserin and the psychedelic is 2-Br-LSD, wherein the volinanserin is administered to post-treat at least 330 minutes post to the 2-Br-LSD. In some embodiments, the serotonin receptor modulator is volinanserin and the psychedelic is 2-Br-LSD, wherein the volinanserin is administered to post-treat at least 360 minutes post to the 2-Br-LSD. In some preferred embodiments, the serotonin receptor modulator is volinanserin and the psychedelic is 2-Br-LSD, wherein volinanserin is administered to post-treat between about 60 minutes and about 180 minutes post to the administration of 2-Br-LSD.

In some embodiments, the serotonin receptor modulator is ketanserin and the psychedelic is 2-Br-LSD, wherein the ketanserin is administered to post-treat at least 15 minutes post to the administration of 2-Br-LSD. In some embodiments, the serotonin receptor modulator is ketanserin and the psychedelic is 2-Br-LSD, wherein the ketanserin is administered to post-treat at least 30 minutes post to the 2-Br-LSD. In some embodiments, the serotonin receptor modulator is ketanserin and the psychedelic is 2-Br-LSD, wherein the ketanserin is administered to post-treat at least 60 minutes post to the 2-Br-LSD. In some embodiments, the serotonin receptor modulator is ketanserin and the psychedelic is 2-Br-LSD, wherein the ketanserin is administered to post-treat between at least 30 minutes and 360 minutes post to the administration or release 2-Br-LSD. In some embodiments, the serotonin receptor modulator is ketanserin and the psychedelic is 2-Br-LSD, wherein the ketanserin is administered to post-treat between at least 60 minutes and 240 minutes post to the administration or release of 2-Br-LSD. In some embodiments, the serotonin receptor modulator is ketanserin and the psychedelic is 2-Br-LSD, wherein the ketanserin is administered to post-treat at least 90 minutes post to the 2-Br-LSD. In some embodiments, the serotonin receptor modulator is ketanserin and the psychedelic is 2-Br-LSD, wherein the ketanserin is administered to post-treat at least 120 minutes post to the 2-Br-LSD. In some embodiments, the serotonin receptor modulator is ketanserin and the psychedelic is 2-Br-LSD, wherein the ketanserin is administered to post-treat at least 150 minutes post to the 2-Br-LSD. In some embodiments, the serotonin receptor modulator is ketanserin and the psychedelic is 2-Br-LSD, wherein the ketanserin is administered to post-treat between about 15 minutes and about 150 minutes post to the 2-Br-LSD. In some embodiments, the serotonin receptor modulator is ketanserin and the psychedelic is 2-Br-LSD, wherein the ketanserin is administered to post-treat at least 180 minutes post to the 2-Br-LSD. In some embodiments, the serotonin receptor modulator is ketanserin and the psychedelic is 2-Br-LSD, wherein the ketanserin is administered to post-treat at least 210 minutes post to the 2-Br-LSD. In some embodiments, the serotonin receptor modulator is ketanserin and the psychedelic is 2-Br-LSD, wherein the ketanserin is administered to post-treat at least 240 minutes post to the 2-Br-LSD. In some embodiments, the serotonin receptor modulator is ketanserin and the psychedelic is 2-Br-LSD, wherein the ketanserin is administered to post-treat at least 270 minutes post to the 2-Br-LSD. In some embodiments, the serotonin receptor modulator is ketanserin and the psychedelic is 2-Br-LSD, wherein the ketanserin is administered to post-treat at least 300 minutes post to the 2-Br-LSD. In some embodiments, the serotonin receptor modulator is ketanserin and the psychedelic is 2-Br-LSD, wherein the ketanserin is administered to post-treat at least 330 minutes post to the 2-Br-LSD. In some embodiments, the serotonin receptor modulator is ketanserin and the psychedelic is 2-Br-LSD, wherein the ketanserin is administered to post-treat at least 360 minutes post to the 2-Br-LSD. In some preferred embodiments, the serotonin receptor modulator is ketanserin and the psychedelic is 2-Br-LSD, wherein ketanserin is administered to post-treat between about 60 minutes and about 180 minutes post to the administration of 2-Br-LSD.

In some embodiments, the serotonin receptor modulator is ritanserin and the psychedelic is 2-Br-LSD, wherein the ritanserin is administered to post-treat at least 15 minutes post to the administration of 2-Br-LSD. In some embodiments, the serotonin receptor modulator is ritanserin and the psychedelic is 2-Br-LSD, wherein the ritanserin is administered to post-treat at least 30 minutes post to the 2-Br-LSD. In some embodiments, the serotonin receptor modulator is ritanserin and the psychedelic is 2-Br-LSD, wherein the ritanserin is administered to post-treat at least 60 minutes post to the 2-Br-LSD. In some embodiments, the serotonin receptor modulator is ritanserin and the psychedelic is 2-Br-LSD, wherein the ritanserin is administered to post-treat between at least 60 minutes and 240 minutes post to the administration or release of 2-Br-LSD. In some embodiments, the serotonin receptor modulator is ritanserin and the psychedelic is 2-Br-LSD, wherein the ritanserin is administered to post-treat at least 90 minutes post to the 2-Br-LSD. In some embodiments, the serotonin receptor modulator is ritanserin and the psychedelic is 2-Br-LSD, wherein the ritanserin is administered to post-treat at least 120 minutes post to the 2-Br-LSD. In some embodiments, the serotonin receptor modulator is ritanserin and the psychedelic is 2-Br-LSD, wherein the ritanserin is administered to post-treat at least 150 minutes post to the 2-Br-LSD. In some embodiments, the serotonin receptor modulator is ritanserin and the psychedelic is 2-Br-LSD, wherein the ritanserin is administered to post-treat between about 15 minutes and about 150 minutes post to the 2-Br-LSD. In some embodiments, the serotonin receptor modulator is ritanserin and the psychedelic is 2-Br-LSD, wherein the ritanserin is administered to post-treat at least 180 minutes post to the 2-Br-LSD. In some embodiments, the serotonin receptor modulator is ritanserin and the psychedelic is 2-Br-LSD, wherein the ritanserin is administered to post-treat at least 210 minutes post to the 2-Br-LSD. In some embodiments, the serotonin receptor modulator is ritanserin and the psychedelic is 2-Br-LSD, wherein the ritanserin is administered to post-treat at least 240 minutes post to the 2-Br-LSD. In some embodiments, the serotonin receptor modulator is ritanserin and the psychedelic is 2-Br-LSD, wherein the ritanserin is administered to post-treat at least 270 minutes post to the 2-Br-LSD. In some embodiments, the serotonin receptor modulator is ritanserin and the psychedelic is 2-Br-LSD, wherein the ritanserin is administered to post-treat at least 300 minutes post to the 2-Br-LSD. In some embodiments, the serotonin receptor modulator is ritanserin and the psychedelic is 2-Br-LSD, wherein the ritanserin is administered to post-treat at least 330 minutes post to the 2-Br-LSD. In some embodiments, the serotonin receptor modulator is ritanserin and the psychedelic is 2-Br-LSD, wherein the ritanserin is administered to post-treat at least 360 minutes post to the 2-Br-LSD. In some preferred embodiments, the serotonin receptor modulator is ritanserin and the psychedelic is 2-Br-LSD, wherein ritanserin is administered to post-treat between about 60 minutes and about 180 minutes post to the administration of 2-Br-LSD.

In some embodiments, the serotonin receptor modulator is pimavanserin and the psychedelic is 2-Br-LSD, wherein the pimavanserin is administered to post-treat at least 15 minutes post to the administration of 2-Br-LSD. In some embodiments, the serotonin receptor modulator is pimavanserin and the psychedelic is 2-Br-LSD, wherein the pimavanserin is administered to post-treat at least 30 minutes post to the 2-Br-LSD. In some embodiments, the serotonin receptor modulator is pimavanserin and the psychedelic is 2-Br-LSD, wherein the pimavanserin is administered to post-treat at least 60 minutes post to the 2-Br-LSD. In some embodiments, the serotonin receptor modulator is pimavanserin and the psychedelic is 2-Br-LSD, wherein the pimavanserin is administered to post-treat between at least 60 minutes and 240 minutes post to the administration or release of 2-Br-LSD. In some embodiments, the serotonin receptor modulator is pimavanserin and the psychedelic is 2-Br-LSD, wherein the pimavanserin is administered to post-treat at least 90 minutes post to the 2-Br-LSD. In some embodiments, the serotonin receptor modulator is pimavanserin and the psychedelic is 2-Br-LSD, wherein the pimavanserin is administered to post-treat at least 120 minutes post to the 2-Br-LSD. In some embodiments, the serotonin receptor modulator is pimavanserin and the psychedelic is 2-Br-LSD, wherein the pimavanserin is administered to post-treat at least 150 minutes post to the 2-Br-LSD. In some embodiments, the serotonin receptor modulator is pimavanserin and the psychedelic is 2-Br-LSD, wherein the pimavanserin is administered to post-treat between about 15 minutes and about 150 minutes post to the 2-Br-LSD. In some embodiments, the serotonin receptor modulator is pimavanserin and the psychedelic is 2-Br-LSD, wherein the pimavanserin is administered to post-treat at least 180 minutes post to the 2-Br-LSD. In some embodiments, the serotonin receptor modulator is pimavanserin and the psychedelic is 2-Br-LSD, wherein the pimavanserin is administered to post-treat at least 210 minutes post to the 2-Br-LSD. In some embodiments, the serotonin receptor modulator is pimavanserin and the psychedelic is 2-Br-LSD, wherein the pimavanserin is administered to post-treat at least 240 minutes post to the 2-Br-LSD. In some embodiments, the serotonin receptor modulator is pimavanserin and the psychedelic is 2-Br-LSD, wherein the pimavanserin is administered to post-treat at least 270 minutes post to the 2-Br-LSD. In some embodiments, the serotonin receptor modulator is pimavanserin and the psychedelic is 2-Br-LSD, wherein the pimavanserin is administered to post-treat at least 300 minutes post to the 2-Br-LSD. In some embodiments, the serotonin receptor modulator is pimavanserin and the psychedelic is 2-Br-LSD, wherein the pimavanserin is administered to post-treat at least 330 minutes post to the 2-Br-LSD. In some embodiments, the serotonin receptor modulator is pimavanserin and the psychedelic is 2-Br-LSD, wherein the pimavanserin is administered to post-treat at least 360 minutes post to the 2-Br-LSD. In some preferred embodiments, the serotonin receptor modulator is pimavanserin and the psychedelic is 2-Br-LSD, wherein pimavanserin is administered to post-treat between about 60 minutes and about 180 minutes post to the administration of 2-Br-LSD.

In some embodiments, the serotonin receptor modulator is nelotanserin and the psychedelic is 2-Br-LSD, wherein the nelotanserin is administered to post-treat at least 15 minutes post to the administration of 2-Br-LSD. In some embodiments, the serotonin receptor modulator is nelotanserin and the psychedelic is 2-Br-LSD, wherein the nelotanserin is administered to post-treat at least 30 minutes post to the 2-Br-LSD. In some embodiments, the serotonin receptor modulator is nelotanserin and the psychedelic is 2-Br-LSD, wherein the nelotanserin is administered to post-treat at least 60 minutes post to the 2-Br-LSD. In some embodiments, the serotonin receptor modulator is nelotanserin and the psychedelic is 2-Br-LSD, wherein the nelotanserin is administered to post-treat between at least 60 minutes and 240 minutes post to the administration or release of 2-Br-LSD. In some embodiments, the serotonin receptor modulator is nelotanserin and the psychedelic is 2-Br-LSD, wherein the nelotanserin is administered to post-treat at least 90 minutes post to the 2-Br-LSD. In some embodiments, the serotonin receptor modulator is nelotanserin and the psychedelic is 2-Br-LSD, wherein the nelotanserin is administered to post-treat at least 120 minutes post to the 2-Br-LSD.

In some embodiments, the serotonin receptor modulator is nelotanserin and the psychedelic is 2-Br-LSD, wherein the nelotanserin is administered to post-treat at least 150 minutes post to the 2-Br-LSD. In some embodiments, the serotonin receptor modulator is nelotanserin and the psychedelic is 2-Br-LSD, wherein the nelotanserin is administered to post-treat between about 15 minutes and about 150 minutes post to the 2-Br-LSD. In some embodiments, the serotonin receptor modulator is nelotanserin and the psychedelic is 2-Br-LSD, wherein the nelotanserin is administered to post-treat at least 180 minutes post to the 2-Br-LSD. In some embodiments, the serotonin receptor modulator is nelotanserin and the psychedelic is 2-Br-LSD, wherein the nelotanserin is administered to post-treat at least 210 minutes post to the 2-Br-LSD. In some embodiments, the serotonin receptor modulator is nelotanserin and the psychedelic is 2-Br-LSD, wherein the nelotanserin is administered to post-treat at least 240 minutes post to the 2-Br-LSD. In some embodiments, the serotonin receptor modulator is nelotanserin and the psychedelic is 2-Br-LSD, wherein the nelotanserin is administered to post-treat at least 270 minutes post to the 2-Br-LSD.

In some embodiments, the serotonin receptor modulator is nelotanserin and the psychedelic is 2-Br-LSD, wherein the nelotanserin is administered to post-treat at least 300 minutes post to the 2-Br-LSD. In some embodiments, the serotonin receptor modulator is nelotanserin and the psychedelic is 2-Br-LSD, wherein the nelotanserin is administered to post-treat at least 330 minutes post to the 2-Br-LSD. In some embodiments, the serotonin receptor modulator is nelotanserin and the psychedelic is 2-Br-LSD, wherein the nelotanserin is administered to post-treat at least 360 minutes post to the 2-Br-LSD. In some preferred embodiments, the serotonin receptor modulator is nelotanserin and the psychedelic is 2-Br-LSD, wherein nelotanserin is administered to post-treat between about 60 minutes and about 180 minutes post to the administration of 2-Br-LSD.

In some embodiments, the serotonin receptor modulator is pruvanserin and the psychedelic is 2-Br-LSD, wherein the pruvanserin is administered to post-treat at least 15 minutes post to the administration of 2-Br-LSD. In some embodiments, the serotonin receptor modulator is pruvanserin and the psychedelic is 2-Br-LSD, wherein the pruvanserin is administered to post-treat at least 30 minutes post to the 2-Br-LSD. In some embodiments, the serotonin receptor modulator is pruvanserin and the psychedelic is 2-Br-LSD, wherein the pruvanserin is administered to post-treat at least 60 minutes post to the 2-Br-LSD. In some embodiments, the serotonin receptor modulator is pruvanserin and the psychedelic is 2-Br-LSD, wherein the pruvanserin is administered to post-treat between at least 60 minutes and 240 minutes post to the administration or release of 2-Br-LSD. In some embodiments, the serotonin receptor modulator is pruvanserin and the psychedelic is 2-Br-LSD, wherein the pruvanserin is administered to post-treat at least 90 minutes post to the 2-Br-LSD. In some embodiments, the serotonin receptor modulator is pruvanserin and the psychedelic is 2-Br-LSD, wherein the pruvanserin is administered to post-treat at least 120 minutes post to the 2-Br-LSD. In some embodiments, the serotonin receptor modulator is pruvanserin and the psychedelic is 2-Br-LSD, wherein the pruvanserin is administered to post-treat at least 150 minutes post to the 2-Br-LSD. In some embodiments, the serotonin receptor modulator is pruvanserin and the psychedelic is 2-Br-LSD, wherein the pruvanserin is administered to post-treat between about 15 minutes and about 150 minutes post to the 2-Br-LSD. In some embodiments, the serotonin receptor modulator is pruvanserin and the psychedelic is 2-Br-LSD, wherein the pruvanserin is administered to post-treat at least 180 minutes post to the 2-Br-LSD. In some embodiments, the serotonin receptor modulator is pruvanserin and the psychedelic is 2-Br-LSD, wherein the pruvanserin is administered to post-treat at least 210 minutes post to the 2-Br-LSD.

In some embodiments, the serotonin receptor modulator is pruvanserin and the psychedelic is 2-Br-LSD, wherein the pruvanserin is administered to post-treat at least 240 minutes post to the 2-Br-LSD. In some embodiments, the serotonin receptor modulator is pruvanserin and the psychedelic is 2-Br-LSD, wherein the pruvanserin is administered to post-treat at least 270 minutes post to the 2-Br-LSD. In some embodiments, the serotonin receptor modulator is pruvanserin and the psychedelic is 2-Br-LSD, wherein the pruvanserin is administered to post-treat at least 300 minutes post to the 2-Br-LSD. In some embodiments, the serotonin receptor modulator is pruvanserin and the psychedelic is 2-Br-LSD, wherein the pruvanserin is administered to post-treat at least 330 minutes post to the 2-Br-LSD. In some embodiments, the serotonin receptor modulator is pruvanserin and the psychedelic is 2-Br-LSD, wherein the pruvanserin is administered to post-treat at least 360 minutes post to the 2-Br-LSD. In some preferred embodiments, the serotonin receptor modulator is pruvanserin and the psychedelic is 2-Br-LSD, wherein pruvanserin is administered to post-treat between about 60 minutes and about 180 minutes post to the administration of 2-Br-LSD.

In some embodiments, the serotonin receptor modulator is flibanserin and the psychedelic is 2-Br-LSD, wherein the flibanserin is administered to post-treat at least 15 minutes post to the administration of 2-Br-LSD. In some embodiments, the serotonin receptor modulator is flibanserin and the psychedelic is 2-Br-LSD, wherein the flibanserin is administered to post-treat at least 30 minutes post to the 2-Br-LSD. In some embodiments, the serotonin receptor modulator is flibanserin and the psychedelic is 2-Br-LSD, wherein the flibanserin is administered to post-treat at least 60 minutes post to the 2-Br-LSD. In some embodiments, the serotonin receptor modulator is flibanserin and the psychedelic is 2-Br-LSD, wherein the flibanserin is administered to post-treat at least 90 minutes post to the 2-Br-LSD. In some embodiments, the serotonin receptor modulator is flibanserin and the psychedelic is 2-Br-LSD, wherein the flibanserin is administered to post-treat at least 120 minutes post to the 2-Br-LSD. In some embodiments, the serotonin receptor modulator is flibanserin and the psychedelic is 2-Br-LSD, wherein the flibanserin is administered to post-treat at least 150 minutes post to the 2-Br-LSD. In some embodiments, the serotonin receptor modulator is flibanserin and the psychedelic is 2-Br-LSD, wherein the flibanserin is administered to post-treat between about 15 minutes and about 150 minutes post to the 2-Br-LSD. In some embodiments, the serotonin receptor modulator is flibanserin and the psychedelic is 2-Br-LSD, wherein the flibanserin is administered to post-treat at least 180 minutes post to the 2-Br-LSD. In some embodiments, the serotonin receptor modulator is flibanserin and the psychedelic is 2-Br-LSD, wherein the flibanserin is administered to post-treat at least 210 minutes post to the 2-Br-LSD. In some embodiments, the serotonin receptor modulator is flibanserin and the psychedelic is 2-Br-LSD, wherein the flibanserin is administered to post-treat at least 240 minutes post to the 2-Br-LSD. In some embodiments, the serotonin receptor modulator is flibanserin and the psychedelic is 2-Br-LSD, wherein the flibanserin is administered to post-treat at least 270 minutes post to the 2-Br-LSD. In some embodiments, the serotonin receptor modulator is flibanserin and the psychedelic is 2-Br-LSD, wherein the flibanserin is administered to post-treat at least 300 minutes post to the 2-Br-LSD. In some embodiments, the serotonin receptor modulator is flibanserin and the psychedelic is 2-Br-LSD, wherein the flibanserin is administered to post-treat at least 330 minutes post to the 2-Br-LSD. In some embodiments, the serotonin receptor modulator is flibanserin and the psychedelic is 2-Br-LSD, wherein the flibanserin is administered to post-treat at least 360 minutes post to the 2-Br-LSD. In some preferred embodiments, the serotonin receptor modulator is flibanserin and the psychedelic is 2-Br-LSD, wherein flibanserin is administered to post-treat between about 60 minutes and about 180 minutes post to the administration of 2-Br-LSD.

In some embodiments, the serotonin receptor modulator is olanzapine and the psychedelic is 2-Br-LSD, wherein the olanzapine is administered to post-treat at least 15 minutes post to the administration of 2-Br-LSD. In some embodiments, the serotonin receptor modulator is olanzapine and the psychedelic is 2-Br-LSD, wherein the olanzapine is administered to post-treat at least 30 minutes post to the 2-Br-LSD. In some embodiments, the serotonin receptor modulator is olanzapine and the psychedelic is 2-Br-LSD, wherein the olanzapine is administered to post-treat at least 60 minutes post to the 2-Br-LSD. In some embodiments, the serotonin receptor modulator is olanzapine and the psychedelic is 2-Br-LSD, wherein the olanzapine is administered to post-treat at least 90 minutes post to the 2-Br-LSD. In some embodiments, the serotonin receptor modulator is olanzapine and the psychedelic is 2-Br-LSD, wherein the olanzapine is administered to post-treat at least 120 minutes post to the 2-Br-LSD. In some embodiments, the serotonin receptor modulator is olanzapine and the psychedelic is 2-Br-LSD, wherein the olanzapine is administered to post-treat at least 150 minutes post to the 2-Br-LSD. In some embodiments, the serotonin receptor modulator is olanzapine and the psychedelic is 2-Br-LSD, wherein the olanzapine is administered to post-treat between about 15 minutes and about 150 minutes post to the 2-Br-LSD. In some embodiments, the serotonin receptor modulator is olanzapine and the psychedelic is 2-Br-LSD, wherein the olanzapine is administered to post-treat at least 180 minutes post to the 2-Br-LSD. In some embodiments, the serotonin receptor modulator is olanzapine and the psychedelic is 2-Br-LSD, wherein the olanzapine is administered to post-treat at least 210 minutes post to the 2-Br-LSD. In some embodiments, the serotonin receptor modulator is olanzapine and the psychedelic is 2-Br-LSD, wherein the olanzapine is administered to post-treat at least 240 minutes post to the 2-Br-LSD. In some embodiments, the serotonin receptor modulator is olanzapine and the psychedelic is 2-Br-LSD, wherein the olanzapine is administered to post-treat at least 270 minutes post to the 2-Br-LSD. In some embodiments, the serotonin receptor modulator is olanzapine and the psychedelic is 2-Br-LSD, wherein the olanzapine is administered to post-treat at least 300 minutes post to the 2-Br-LSD. In some embodiments, the serotonin receptor modulator is olanzapine and the psychedelic is 2-Br-LSD, wherein the olanzapine is administered to post-treat at least 330 minutes post to the 2-Br-LSD. In some embodiments, the serotonin receptor modulator is olanzapine and the psychedelic is 2-Br-LSD, wherein the olanzapine is administered to post-treat at least 360 minutes post to the 2-Br-LSD. In some preferred embodiments, the serotonin receptor modulator is olanzapine and the psychedelic is 2-Br-LSD, wherein olanzapine is administered to post-treat between about 60 minutes and about 180 minutes post to the administration of 2-Br-LSD.

In some embodiments, the serotonin receptor modulator is quetiapine and the psychedelic is 2-Br-LSD, wherein the quetiapine is administered to post-treat at least 15 minutes post to the administration of 2-Br-LSD. In some embodiments, the serotonin receptor modulator is quetiapine and the psychedelic is 2-Br-LSD, wherein the quetiapine is administered to post-treat at least 30 minutes post to the 2-Br-LSD. In some embodiments, the serotonin receptor modulator is quetiapine and the psychedelic is 2-Br-LSD, wherein the quetiapine is administered to post-treat at least 60 minutes post to the 2-Br-LSD. In some embodiments, the serotonin receptor modulator is quetiapine and the psychedelic is 2-Br-LSD, wherein the quetiapine is administered to post-treat at least 90 minutes post to the 2-Br-LSD. In some embodiments, the serotonin receptor modulator is quetiapine and the psychedelic is 2-Br-LSD, wherein the quetiapine is administered to post-treat at least 120 minutes post to the 2-Br-LSD. In some embodiments, the serotonin receptor modulator is quetiapine and the psychedelic is 2-Br-LSD, wherein the quetiapine is administered to post-treat at least 150 minutes post to the 2-Br-LSD. In some embodiments, the serotonin receptor modulator is quetiapine and the psychedelic is 2-Br-LSD, wherein the quetiapine is administered to post-treat between about 15 minutes and about 150 minutes post to the 2-Br-LSD. In some embodiments, the serotonin receptor modulator is quetiapine and the psychedelic is 2-Br-LSD, wherein the quetiapine is administered to post-treat at least 180 minutes post to the 2-Br-LSD. In some embodiments, the serotonin receptor modulator is quetiapine and the psychedelic is 2-Br-LSD, wherein the quetiapine is administered to post-treat at least 210 minutes post to the 2-Br-LSD. In some embodiments, the serotonin receptor modulator is quetiapine and the psychedelic is 2-Br-LSD, wherein the quetiapine is administered to post-treat at least 240 minutes post to the 2-Br-LSD. In some embodiments, the serotonin receptor modulator is quetiapine and the psychedelic is 2-Br-LSD, wherein the quetiapine is administered to post-treat at least 270 minutes post to the 2-Br-LSD. In some embodiments, the serotonin receptor modulator is quetiapine and the psychedelic is 2-Br-LSD, wherein the quetiapine is administered to post-treat at least 300 minutes post to the 2-Br-LSD. In some embodiments, the serotonin receptor modulator is quetiapine and the psychedelic is 2-Br-LSD, wherein the quetiapine is administered to post-treat at least 330 minutes post to the 2-Br-LSD. In some embodiments, the serotonin receptor modulator is quetiapine and the psychedelic is 2-Br-LSD, wherein the quetiapine is administered to post-treat at least 360 minutes post to the 2-Br-LSD. In some preferred embodiments, the serotonin receptor modulator is quetiapine and the psychedelic is 2-Br-LSD, wherein quetiapine is administered to post-treat between about 60 minutes and about 180 minutes post to the administration of 2-Br-LSD.

In some embodiments, the serotonin receptor modulator is risperidone and the psychedelic is 2-Br-LSD, wherein the risperidone is administered to post-treat at least 15 minutes post to the administration of 2-Br-LSD. In some embodiments, the serotonin receptor modulator is risperidone and the psychedelic is 2-Br-LSD, wherein the risperidone is administered to post-treat at least 30 minutes post to the 2-Br-LSD. In some embodiments, the serotonin receptor modulator is risperidone and the psychedelic is 2-Br-LSD, wherein the risperidone is administered to post-treat at least 60 minutes post to the 2-Br-LSD. In some embodiments, the serotonin receptor modulator is risperidone and the psychedelic is 2-Br-LSD, wherein the risperidone is administered to post-treat at least 90 minutes post to the 2-Br-LSD. In some embodiments, the serotonin receptor modulator is risperidone and the psychedelic is 2-Br-LSD, wherein the risperidone is administered to post-treat at least 120 minutes post to the 2-Br-LSD. In some embodiments, the serotonin receptor modulator is risperidone and the psychedelic is 2-Br-LSD, wherein the risperidone is administered to post-treat at least 150 minutes post to the 2-Br-LSD. In some embodiments, the serotonin receptor modulator is risperidone and the psychedelic is 2-Br-LSD, wherein the risperidone is administered to post-treat between about 15 minutes and about 150 minutes post to the 2-Br-LSD. In some embodiments, the serotonin receptor modulator is risperidone and the psychedelic is 2-Br-LSD, wherein the risperidone is administered to post-treat at least 180 minutes post to the 2-Br-LSD. In some embodiments, the serotonin receptor modulator is risperidone and the psychedelic is 2-Br-LSD, wherein the risperidone is administered to post-treat at least 210 minutes post to the 2-Br-LSD. In some embodiments, the serotonin receptor modulator is risperidone and the psychedelic is 2-Br-LSD, wherein the risperidone is administered to post-treat at least 240 minutes post to the 2-Br-LSD. In some embodiments, the serotonin receptor modulator is risperidone and the psychedelic is 2-Br-LSD, wherein the risperidone is administered to post-treat at least 270 minutes post to the 2-Br-LSD. In some embodiments, the serotonin receptor modulator is risperidone and the psychedelic is 2-Br-LSD, wherein the risperidone is administered to post-treat at least 300 minutes post to the 2-Br-LSD. In some embodiments, the serotonin receptor modulator is risperidone and the psychedelic is 2-Br-LSD, wherein the risperidone is administered to post-treat at least 330 minutes post to the 2-Br-LSD. In some embodiments, the serotonin receptor modulator is risperidone and the psychedelic is 2-Br-LSD, wherein the risperidone is administered to post-treat at least 360 minutes post to the 2-Br-LSD. In some preferred embodiments, the serotonin receptor modulator is risperidone and the psychedelic is 2-Br-LSD, wherein risperidone is administered to post-treat between about 60 minutes and about 180 minutes post to the administration of 2-Br-LSD.

In some embodiments, the serotonin receptor modulator is eplivanserin and the psychedelic is MDAI, wherein the eplivanserin is administered to post-treat at least 15 minutes post to the administration of MDAI. In some embodiments, the serotonin receptor modulator is eplivanserin and the psychedelic is MDAI, wherein the eplivanserin is administered to post-treat at least 30 minutes post to the MDAI. In some embodiments, the serotonin receptor modulator is eplivanserin and the psychedelic is MDAI, wherein the eplivanserin is administered to post-treat at least 60 minutes post to the MDAI. In some embodiments, the serotonin receptor modulator is eplivanserin and the psychedelic is MDAI, wherein the eplivanserin is administered to post-treat at least 90 minutes post to the MDAI. In some embodiments, the serotonin receptor modulator is eplivanserin and the psychedelic is MDAI, wherein the eplivanserin is administered to post-treat between at least 30 minutes post and 360 minutes post to the release or administration of the MDAI. In some embodiments, the serotonin receptor modulator is eplivanserin and the psychedelic is MDAI, wherein the eplivanserin is administered to post-treat between at least 60 minutes post and 360 minutes post to the release or administration the MDAI. In some embodiments, the serotonin receptor modulator is eplivanserin and the psychedelic is MDAI, wherein the eplivanserin is administered to post-treat between at least 90 minutes and 240 minutes post to the MDAI. In some embodiments, the serotonin receptor modulator is eplivanserin and the psychedelic is MDAI, wherein the eplivanserin is administered to post-treat at least 120 minutes post to the MDAI. In some embodiments, the serotonin receptor modulator is eplivanserin and the psychedelic is MDAI, wherein the eplivanserin is administered to post-treat at least 150 minutes post to the MDAI. In some embodiments, the serotonin receptor modulator is eplivanserin and the psychedelic is MDAI, wherein the eplivanserin is administered to post-treat between about 15 minutes and about 150 minutes post to the MDAI In some embodiments, the serotonin receptor modulator is eplivanserin and the psychedelic is MDAI, wherein the eplivanserin is administered to post-treat at least 180 minutes post to the MDAI. In some embodiments, the serotonin receptor modulator is eplivanserin and the psychedelic is MDAI, wherein the eplivanserin is administered to post-treat at least 210 minutes post to the MDAI. In some embodiments, the serotonin receptor modulator is eplivanserin and the psychedelic is MDAI, wherein the eplivanserin is administered to post-treat at least 240 minutes post to the MDAI.

In some embodiments, the serotonin receptor modulator is eplivanserin and the psychedelic is MDAI, wherein the eplivanserin is administered to post-treat at least 270 minutes post to the MDAI. In some embodiments, the serotonin receptor modulator is eplivanserin and the psychedelic is MDAI, wherein the eplivanserin is administered to post-treat at least 300 minutes post to the MDAI. In some embodiments, the serotonin receptor modulator is eplivanserin and the psychedelic is MDAI, wherein the eplivanserin is administered to post-treat at least 330 minutes post to the MDAI. In some embodiments, the serotonin receptor modulator is eplivanserin and the psychedelic is MDAI, wherein the eplivanserin is administered to post-treat at least 360 minutes post to the MDAI.

In some preferred embodiments, the serotonin receptor modulator is eplivanserin and the psychedelic is MDAI, wherein eplivanserin is administered to post-treat between about 60 minutes and about 180 minutes post to the administration of MDAI.

In some embodiments, the serotonin receptor modulator is volinanserin and the psychedelic is MDAI, wherein the volinanserin is administered to post-treat at least 15 minutes post to the administration of MDAI. In some embodiments, the serotonin receptor modulator is volinanserin and the psychedelic is MDAI, wherein the volinanserin is administered to post-treat at least 30 minutes post to the MDAI. In some embodiments, the serotonin receptor modulator is volinanserin and the psychedelic is MDAI, wherein the volinanserin is administered to post-treat at least 60 minutes post to the MDAI. In some embodiments, the serotonin receptor modulator is volinanserin and the psychedelic is MDAI, wherein the volinanserin is administered to post-treat a subject between at least 15 minutes and 360 minutes post to the administration or release of MDAI. In some embodiments, the serotonin receptor modulator is volinanserin and the psychedelic is MDAI, wherein the volinanserin is administered to post-treat between at least 30 minutes and 360 minutes post to the administration or release of MDAI. In some embodiments, the serotonin receptor modulator is volinanserin and the psychedelic is MDAI, wherein the volinanserin is administered to post-treat between at least 60 minutes and 240 minutes post to the administration or release of MDAI. In some embodiments, the serotonin receptor modulator is volinanserin and the psychedelic is MDAI, wherein the volinanserin is administered to post-treat at least 90 minutes post to the MDAI. In some embodiments, the serotonin receptor modulator is volinanserin and the psychedelic is MDAI, wherein the volinanserin is administered to post-treat at least 120 minutes post to the MDAI.

In some embodiments, the serotonin receptor modulator is volinanserin and the psychedelic is MDAI, wherein the volinanserin is administered to post-treat at least 150 minutes post to the MDAI. In some embodiments, the serotonin receptor modulator is volinanserin and the psychedelic is MDAI, wherein the volinanserin is administered to post-treat between about 15 minutes and about 150 minutes post to the MDAI. In some embodiments, the serotonin receptor modulator is volinanserin and the psychedelic is MDAI, wherein the volinanserin is administered to post-treat at least 180 minutes post to the MDAI. In some embodiments, the serotonin receptor modulator is volinanserin and the psychedelic is MDAI, wherein the volinanserin is administered to post-treat at least 210 minutes post to the MDAI. In some embodiments, the serotonin receptor modulator is volinanserin and the psychedelic is MDAI, wherein the volinanserin is administered to post-treat at least 240 minutes post to the MDAI. In some embodiments, the serotonin receptor modulator is volinanserin and the psychedelic is MDAI, wherein the volinanserin is administered to post-treat at least 270 minutes post to the MDAI. In some embodiments, the serotonin receptor modulator is volinanserin and the psychedelic is MDAI, wherein the volinanserin is administered to post-treat at least 300 minutes post to the MDAI. In some embodiments, the serotonin receptor modulator is volinanserin and the psychedelic is MDAI, wherein the volinanserin is administered to post-treat at least 330 minutes post to the MDAI. In some embodiments, the serotonin receptor modulator is volinanserin and the psychedelic is MDAI, wherein the volinanserin is administered to post-treat at least 360 minutes post to the MDAI. In some preferred embodiments, the serotonin receptor modulator is volinanserin and the psychedelic is MDAI, wherein volinanserin is administered to post-treat between about 60 minutes and about 180 minutes post to the administration of MDAI.

In some embodiments, the serotonin receptor modulator is ketanserin and the psychedelic is MDAI, wherein the ketanserin is administered to post-treat at least 15 minutes post to the administration of MDAI. In some embodiments, the serotonin receptor modulator is ketanserin and the psychedelic is MDAI, wherein the ketanserin is administered to post-treat at least 30 minutes post to the MDAI. In some embodiments, the serotonin receptor modulator is ketanserin and the psychedelic is MDAI, wherein the ketanserin is administered to post-treat at least 60 minutes post to the MDAI. In some embodiments, the serotonin receptor modulator is ketanserin and the psychedelic is MDAI, wherein the ketanserin is administered to post-treat between at least 30 minutes and 360 minutes post to the administration or release MDAI. In some embodiments, the serotonin receptor modulator is ketanserin and the psychedelic is MDAI, wherein the ketanserin is administered to post-treat between at least 60 minutes and 240 minutes post to the administration or release of MDAI. In some embodiments, the serotonin receptor modulator is ketanserin and the psychedelic is MDAI, wherein the ketanserin is administered to post-treat at least 90 minutes post to the MDAI. In some embodiments, the serotonin receptor modulator is ketanserin and the psychedelic is MDAI, wherein the ketanserin is administered to post-treat at least 120 minutes post to the MDAI. In some embodiments, the serotonin receptor modulator is ketanserin and the psychedelic is MDAI, wherein the ketanserin is administered to post-treat at least 150 minutes post to the MDAI. In some embodiments, the serotonin receptor modulator is ketanserin and the psychedelic is MDAI, wherein the ketanserin is administered to post-treat between about 15 minutes and about 150 minutes post to the MDAI. In some embodiments, the serotonin receptor modulator is ketanserin and the psychedelic is MDAI, wherein the ketanserin is administered to post-treat at least 180 minutes post to the MDAI. In some embodiments, the serotonin receptor modulator is ketanserin and the psychedelic is MDAI, wherein the ketanserin is administered to post-treat at least 210 minutes post to the MDAI. In some embodiments, the serotonin receptor modulator is ketanserin and the psychedelic is MDAI, wherein the ketanserin is administered to post-treat at least 240 minutes post to the MDAI. In some embodiments, the serotonin receptor modulator is ketanserin and the psychedelic is MDAI, wherein the ketanserin is administered to post-treat at least 270 minutes post to the MDAI. In some embodiments, the serotonin receptor modulator is ketanserin and the psychedelic is MDAI, wherein the ketanserin is administered to post-treat at least 300 minutes post to the MDAI. In some embodiments, the serotonin receptor modulator is ketanserin and the psychedelic is MDAI, wherein the ketanserin is administered to post-treat at least 330 minutes post to the MDAI. In some embodiments, the serotonin receptor modulator is ketanserin and the psychedelic is MDAI, wherein the ketanserin is administered to post-treat at least 360 minutes post to the MDAI. In some preferred embodiments, the serotonin receptor modulator is ketanserin and the psychedelic is MDAI, wherein ketanserin is administered to post-treat between about 60 minutes and about 180 minutes post to the administration of MDAI.

In some embodiments, the serotonin receptor modulator is ritanserin and the psychedelic is MDAI, wherein the ritanserin is administered to post-treat at least 15 minutes post to the administration of MDAI. In some embodiments, the serotonin receptor modulator is ritanserin and the psychedelic is MDAI, wherein the ritanserin is administered to post-treat at least 30 minutes post to the MDAI. In some embodiments, the serotonin receptor modulator is ritanserin and the psychedelic is MDAI, wherein the ritanserin is administered to post-treat at least 60 minutes post to the MDAI. In some embodiments, the serotonin receptor modulator is ritanserin and the psychedelic is MDAI, wherein the ritanserin is administered to post-treat between at least 60 minutes and 240 minutes post to the administration or release of MDAI. In some embodiments, the serotonin receptor modulator is ritanserin and the psychedelic is MDAI, wherein the ritanserin is administered to post-treat at least 90 minutes post to the MDAI. In some embodiments, the serotonin receptor modulator is ritanserin and the psychedelic is MDAI, wherein the ritanserin is administered to post-treat at least 120 minutes post to the MDAI. In some embodiments, the serotonin receptor modulator is ritanserin and the psychedelic is MDAI, wherein the ritanserin is administered to post-treat at least 150 minutes post to the MDAI. In some embodiments, the serotonin receptor modulator is ritanserin and the psychedelic is MDAI, wherein the ritanserin is administered to post-treat between about 15 minutes and about 150 minutes post to the MDAI. In some embodiments, the serotonin receptor modulator is ritanserin and the psychedelic is MDAI, wherein the ritanserin is administered to post-treat at least 180 minutes post to the MDAI. In some embodiments, the serotonin receptor modulator is ritanserin and the psychedelic is MDAI, wherein the ritanserin is administered to post-treat at least 210 minutes post to the MDAI. In some embodiments, the serotonin receptor modulator is ritanserin and the psychedelic is MDAI, wherein the ritanserin is administered to post-treat at least 240 minutes post to the MDAI. In some embodiments, the serotonin receptor modulator is ritanserin and the psychedelic is MDAI, wherein the ritanserin is administered to post-treat at least 270 minutes post to the MDAI. In some embodiments, the serotonin receptor modulator is ritanserin and the psychedelic is MDAI, wherein the ritanserin is administered to post-treat at least 300 minutes post to the MDAI. In some embodiments, the serotonin receptor modulator is ritanserin and the psychedelic is MDAI, wherein the ritanserin is administered to post-treat at least 330 minutes post to the MDAI. In some embodiments, the serotonin receptor modulator is ritanserin and the psychedelic is MDAI, wherein the ritanserin is administered to post-treat at least 360 minutes post to the MDAI. In some preferred embodiments, the serotonin receptor modulator is ritanserin and the psychedelic is MDAI, wherein ritanserin is administered to post-treat between about 60 minutes and about 180 minutes post to the administration of MDAI.

In some embodiments, the serotonin receptor modulator is pimavanserin and the psychedelic is MDAI, wherein the pimavanserin is administered to post-treat at least 15 minutes post to the administration of MDAI. In some embodiments, the serotonin receptor modulator is pimavanserin and the psychedelic is MDAI, wherein the pimavanserin is administered to post-treat at least 30 minutes post to the MDAI. In some embodiments, the serotonin receptor modulator is pimavanserin and the psychedelic is MDAI, wherein the pimavanserin is administered to post-treat at least 60 minutes post to the MDAI. In some embodiments, the serotonin receptor modulator is pimavanserin and the psychedelic is MDAI, wherein the pimavanserin is administered to post-treat between at least 60 minutes and 240 minutes post to the administration or release of MDAI. In some embodiments, the serotonin receptor modulator is pimavanserin and the psychedelic is MDAI, wherein the pimavanserin is administered to post-treat at least 90 minutes post to the MDAI. In some embodiments, the serotonin receptor modulator is pimavanserin and the psychedelic is MDAI, wherein the pimavanserin is administered to post-treat at least 120 minutes post to the MDAI. In some embodiments, the serotonin receptor modulator is pimavanserin and the psychedelic is MDAI, wherein the pimavanserin is administered to post-treat at least 150 minutes post to the MDAI. In some embodiments, the serotonin receptor modulator is pimavanserin and the psychedelic is MDAI, wherein the pimavanserin is administered to post-treat between about 15 minutes and about 150 minutes post to the MDAI. In some embodiments, the serotonin receptor modulator is pimavanserin and the psychedelic is MDAI, wherein the pimavanserin is administered to post-treat at least 180 minutes post to the MDAI. In some embodiments, the serotonin receptor modulator is pimavanserin and the psychedelic is MDAI, wherein the pimavanserin is administered to post-treat at least 210 minutes post to the MDAI. In some embodiments, the serotonin receptor modulator is pimavanserin and the psychedelic is MDAI, wherein the pimavanserin is administered to post-treat at least 240 minutes post to the MDAI. In some embodiments, the serotonin receptor modulator is pimavanserin and the psychedelic is MDAI, wherein the pimavanserin is administered to post-treat at least 270 minutes post to the MDAI. In some embodiments, the serotonin receptor modulator is pimavanserin and the psychedelic is MDAI, wherein the pimavanserin is administered to post-treat at least 300 minutes post to the MDAI. In some embodiments, the serotonin receptor modulator is pimavanserin and the psychedelic is MDAI, wherein the pimavanserin is administered to post-treat at least 330 minutes post to the MDAI. In some embodiments, the serotonin receptor modulator is pimavanserin and the psychedelic is MDAI, wherein the pimavanserin is administered to post-treat at least 360 minutes post to the MDAI. In some preferred embodiments, the serotonin receptor modulator is pimavanserin and the psychedelic is MDAI, wherein pimavanserin is administered to post-treat between about 60 minutes and about 180 minutes post to the administration of MDAI.

In some embodiments, the serotonin receptor modulator is nelotanserin and the psychedelic is MDAI, wherein the nelotanserin is administered to post-treat at least 15 minutes post to the administration of MDAI. In some embodiments, the serotonin receptor modulator is nelotanserin and the psychedelic is MDAI, wherein the nelotanserin is administered to post-treat at least 30 minutes post to the MDAI. In some embodiments, the serotonin receptor modulator is nelotanserin and the psychedelic is MDAI, wherein the nelotanserin is administered to post-treat at least 60 minutes post to the MDAI. In some embodiments, the serotonin receptor modulator is nelotanserin and the psychedelic is MDAI, wherein the nelotanserin is administered to post-treat between at least 60 minutes and 240 minutes post to the administration or release of MDAI. In some embodiments, the serotonin receptor modulator is nelotanserin and the psychedelic is MDAI, wherein the nelotanserin is administered to post-treat at least 90 minutes post to the MDAI. In some embodiments, the serotonin receptor modulator is nelotanserin and the psychedelic is MDAI, wherein the nelotanserin is administered to post-treat at least 120 minutes post to the MDAI.

In some embodiments, the serotonin receptor modulator is nelotanserin and the psychedelic is MDAI, wherein the nelotanserin is administered to post-treat at least 150 minutes post to the MDAI. In some embodiments, the serotonin receptor modulator is nelotanserin and the psychedelic is MDAI, wherein the nelotanserin is administered to post-treat between about 15 minutes and about 150 minutes post to the MDAI. In some embodiments, the serotonin receptor modulator is nelotanserin and the psychedelic is MDAI, wherein the nelotanserin is administered to post-treat at least 180 minutes post to the MDAI. In some embodiments, the serotonin receptor modulator is nelotanserin and the psychedelic is MDAI, wherein the nelotanserin is administered to post-treat at least 210 minutes post to the MDAI. In some embodiments, the serotonin receptor modulator is nelotanserin and the psychedelic is MDAI, wherein the nelotanserin is administered to post-treat at least 240 minutes post to the MDAI. In some embodiments, the serotonin receptor modulator is nelotanserin and the psychedelic is MDAI, wherein the nelotanserin is administered to post-treat at least 270 minutes post to the MDAI.

In some embodiments, the serotonin receptor modulator is nelotanserin and the psychedelic is MDAI, wherein the nelotanserin is administered to post-treat at least 300 minutes post to the MDAI. In some embodiments, the serotonin receptor modulator is nelotanserin and the psychedelic is MDAI, wherein the nelotanserin is administered to post-treat at least 330 minutes post to the MDAI. In some embodiments, the serotonin receptor modulator is nelotanserin and the psychedelic is MDAI, wherein the nelotanserin is administered to post-treat at least 360 minutes post to the MDAI. In some preferred embodiments, the serotonin receptor modulator is nelotanserin and the psychedelic is MDAI, wherein nelotanserin is administered to post-treat between about 60 minutes and about 180 minutes post to the administration of MDAI.

In some embodiments, the serotonin receptor modulator is pruvanserin and the psychedelic is MDAI, wherein the pruvanserin is administered to post-treat at least 15 minutes post to the administration of MDAI. In some embodiments, the serotonin receptor modulator is pruvanserin and the psychedelic is MDAI, wherein the pruvanserin is administered to post-treat at least 30 minutes post to the MDAI. In some embodiments, the serotonin receptor modulator is pruvanserin and the psychedelic is MDAI, wherein the pruvanserin is administered to post-treat at least 60 minutes post to the MDAI. In some embodiments, the serotonin receptor modulator is pruvanserin and the psychedelic is MDAI, wherein the pruvanserin is administered to post-treat between at least 60 minutes and 240 minutes post to the administration or release of MDAI. In some embodiments, the serotonin receptor modulator is pruvanserin and the psychedelic is MDAI, wherein the pruvanserin is administered to post-treat at least 90 minutes post to the MDAI. In some embodiments, the serotonin receptor modulator is pruvanserin and the psychedelic is MDAI, wherein the pruvanserin is administered to post-treat at least 120 minutes post to the MDAI. In some embodiments, the serotonin receptor modulator is pruvanserin and the psychedelic is MDAI, wherein the pruvanserin is administered to post-treat at least 150 minutes post to the MDAI. In some embodiments, the serotonin receptor modulator is pruvanserin and the psychedelic is MDAI, wherein the pruvanserin is administered to post-treat between about 15 minutes and about 150 minutes post to the MDAI. In some embodiments, the serotonin receptor modulator is pruvanserin and the psychedelic is MDAI, wherein the pruvanserin is administered to post-treat at least 180 minutes post to the MDAI. In some embodiments, the serotonin receptor modulator is pruvanserin and the psychedelic is MDAI, wherein the pruvanserin is administered to post-treat at least 210 minutes post to the MDAI.

In some embodiments, the serotonin receptor modulator is pruvanserin and the psychedelic is MDAI, wherein the pruvanserin is administered to post-treat at least 240 minutes post to the MDAI. In some embodiments, the serotonin receptor modulator is pruvanserin and the psychedelic is MDAI, wherein the pruvanserin is administered to post-treat at least 270 minutes post to the MDAI. In some embodiments, the serotonin receptor modulator is pruvanserin and the psychedelic is MDAI, wherein the pruvanserin is administered to post-treat at least 300 minutes post to the MDAI. In some embodiments, the serotonin receptor modulator is pruvanserin and the psychedelic is MDAI, wherein the pruvanserin is administered to post-treat at least 330 minutes post to the MDAI. In some embodiments, the serotonin receptor modulator is pruvanserin and the psychedelic is MDAI, wherein the pruvanserin is administered to post-treat at least 360 minutes post to the MDAI. In some preferred embodiments, the serotonin receptor modulator is pruvanserin and the psychedelic is MDAI, wherein pruvanserin is administered to post-treat between about 60 minutes and about 180 minutes post to the administration of MDAI.

In some embodiments, the serotonin receptor modulator is flibanserin and the psychedelic is MDAI, wherein the flibanserin is administered to post-treat at least 15 minutes post to the administration of MDAI. In some embodiments, the serotonin receptor modulator is flibanserin and the psychedelic is MDAI, wherein the flibanserin is administered to post-treat at least 30 minutes post to the MDAI. In some embodiments, the serotonin receptor modulator is flibanserin and the psychedelic is MDAI, wherein the flibanserin is administered to post-treat at least 60 minutes post to the MDAI. In some embodiments, the serotonin receptor modulator is flibanserin and the psychedelic is MDAI, wherein the flibanserin is administered to post-treat at least 90 minutes post to the MDAI. In some embodiments, the serotonin receptor modulator is flibanserin and the psychedelic is MDAI, wherein the flibanserin is administered to post-treat at least 120 minutes post to the MDAI. In some embodiments, the serotonin receptor modulator is flibanserin and the psychedelic is MDAI, wherein the flibanserin is administered to post-treat at least 150 minutes post to the MDAI. In some embodiments, the serotonin receptor modulator is flibanserin and the psychedelic is MDAI, wherein the flibanserin is administered to post-treat between about 15 minutes and about 150 minutes post to the MDAI. In some embodiments, the serotonin receptor modulator is flibanserin and the psychedelic is MDAI, wherein the flibanserin is administered to post-treat at least 180 minutes post to the MDAI. In some embodiments, the serotonin receptor modulator is flibanserin and the psychedelic is MDAI, wherein the flibanserin is administered to post-treat at least 210 minutes post to the MDAI. In some embodiments, the serotonin receptor modulator is flibanserin and the psychedelic is MDAI, wherein the flibanserin is administered to post-treat at least 240 minutes post to the MDAI. In some embodiments, the serotonin receptor modulator is flibanserin and the psychedelic is MDAI, wherein the flibanserin is administered to post-treat at least 270 minutes post to the MDAI. In some embodiments, the serotonin receptor modulator is flibanserin and the psychedelic is MDAI, wherein the flibanserin is administered to post-treat at least 300 minutes post to the MDAI. In some embodiments, the serotonin receptor modulator is flibanserin and the psychedelic is MDAI, wherein the flibanserin is administered to post-treat at least 330 minutes post to the MDAI. In some embodiments, the serotonin receptor modulator is flibanserin and the psychedelic is MDAI, wherein the flibanserin is administered to post-treat at least 360 minutes post to the MDAI. In some preferred embodiments, the serotonin receptor modulator is flibanserin and the psychedelic is MDAI, wherein flibanserin is administered to post-treat between about 60 minutes and about 180 minutes post to the administration of MDAI.

In some embodiments, the serotonin receptor modulator is olanzapine and the psychedelic is MDAI, wherein the olanzapine is administered to post-treat at least 15 minutes post to the administration of MDAI. In some embodiments, the serotonin receptor modulator is olanzapine and the psychedelic is MDAI, wherein the olanzapine is administered to post-treat at least 30 minutes post to the MDAI. In some embodiments, the serotonin receptor modulator is olanzapine and the psychedelic is MDAI, wherein the olanzapine is administered to post-treat at least 60 minutes post to the MDAI. In some embodiments, the serotonin receptor modulator is olanzapine and the psychedelic is MDAI, wherein the olanzapine is administered to post-treat at least 90 minutes post to the MDAI. In some embodiments, the serotonin receptor modulator is olanzapine and the psychedelic is MDAI, wherein the olanzapine is administered to post-treat at least 120 minutes post to the MDAI. In some embodiments, the serotonin receptor modulator is olanzapine and the psychedelic is MDAI, wherein the olanzapine is administered to post-treat at least 150 minutes post to the MDAI. In some embodiments, the serotonin receptor modulator is olanzapine and the psychedelic is MDAI, wherein the olanzapine is administered to post-treat between about 15 minutes and about 150 minutes post to the MDAI. In some embodiments, the serotonin receptor modulator is olanzapine and the psychedelic is MDAI, wherein the olanzapine is administered to post-treat at least 180 minutes post to the MDAI. In some embodiments, the serotonin receptor modulator is olanzapine and the psychedelic is MDAI, wherein the olanzapine is administered to post-treat at least 210 minutes post to the MDAI. In some embodiments, the serotonin receptor modulator is olanzapine and the psychedelic is MDAI, wherein the olanzapine is administered to post-treat at least 240 minutes post to the MDAI. In some embodiments, the serotonin receptor modulator is olanzapine and the psychedelic is MDAI, wherein the olanzapine is administered to post-treat at least 270 minutes post to the MDAI. In some embodiments, the serotonin receptor modulator is olanzapine and the psychedelic is MDAI, wherein the olanzapine is administered to post-treat at least 300 minutes post to the MDAI. In some embodiments, the serotonin receptor modulator is olanzapine and the psychedelic is MDAI, wherein the olanzapine is administered to post-treat at least 330 minutes post to the MDAI. In some embodiments, the serotonin receptor modulator is olanzapine and the psychedelic is MDAI, wherein the olanzapine is administered to post-treat at least 360 minutes post to the MDAI. In some preferred embodiments, the serotonin receptor modulator is olanzapine and the psychedelic is MDAI, wherein olanzapine is administered to post-treat between about 60 minutes and about 180 minutes post to the administration of MDAI.

In some embodiments, the serotonin receptor modulator is quetiapine and the psychedelic is MDAI, wherein the quetiapine is administered to post-treat at least 15 minutes post to the administration of MDAI. In some embodiments, the serotonin receptor modulator is quetiapine and the psychedelic is MDAI, wherein the quetiapine is administered to post-treat at least 30 minutes post to the MDAI. In some embodiments, the serotonin receptor modulator is quetiapine and the psychedelic is MDAI, wherein the quetiapine is administered to post-treat at least 60 minutes post to the MDAI. In some embodiments, the serotonin receptor modulator is quetiapine and the psychedelic is MDAI, wherein the quetiapine is administered to post-treat at least 90 minutes post to the MDAI. In some embodiments, the serotonin receptor modulator is quetiapine and the psychedelic is MDAI, wherein the quetiapine is administered to post-treat at least 120 minutes post to the MDAI. In some embodiments, the serotonin receptor modulator is quetiapine and the psychedelic is MDAI, wherein the quetiapine is administered to post-treat at least 150 minutes post to the MDAI. In some embodiments, the serotonin receptor modulator is quetiapine and the psychedelic is MDAI, wherein the quetiapine is administered to post-treat between about 15 minutes and about 150 minutes post to the MDAI. In some embodiments, the serotonin receptor modulator is quetiapine and the psychedelic is MDAI, wherein the quetiapine is administered to post-treat at least 180 minutes post to the MDAI. In some embodiments, the serotonin receptor modulator is quetiapine and the psychedelic is MDAI, wherein the quetiapine is administered to post-treat at least 210 minutes post to the MDAI. In some embodiments, the serotonin receptor modulator is quetiapine and the psychedelic is MDAI, wherein the quetiapine is administered to post-treat at least 240 minutes post to the MDAI. In some embodiments, the serotonin receptor modulator is quetiapine and the psychedelic is MDAI, wherein the quetiapine is administered to post-treat at least 270 minutes post to the MDAI. In some embodiments, the serotonin receptor modulator is quetiapine and the psychedelic is MDAI, wherein the quetiapine is administered to post-treat at least 300 minutes post to the MDAI. In some embodiments, the serotonin receptor modulator is quetiapine and the psychedelic is MDAI, wherein the quetiapine is administered to post-treat at least 330 minutes post to the MDAI. In some embodiments, the serotonin receptor modulator is quetiapine and the psychedelic is MDAI, wherein the quetiapine is administered to post-treat at least 360 minutes post to the MDAI. In some preferred embodiments, the serotonin receptor modulator is quetiapine and the psychedelic is MDAI, wherein quetiapine is administered to post-treat between about 60 minutes and about 180 minutes post to the administration of MDAI.

In some embodiments, the serotonin receptor modulator is risperidone and the psychedelic is MDAI, wherein the risperidone is administered to post-treat at least 15 minutes post to the administration of MDAI. In some embodiments, the serotonin receptor modulator is risperidone and the psychedelic is MDAI, wherein the risperidone is administered to post-treat at least 30 minutes post to the MDAI. In some embodiments, the serotonin receptor modulator is risperidone and the psychedelic is MDAI, wherein the risperidone is administered to post-treat at least 60 minutes post to the MDAI. In some embodiments, the serotonin receptor modulator is risperidone and the psychedelic is MDAI, wherein the risperidone is administered to post-treat at least 90 minutes post to the MDAI. In some embodiments, the serotonin receptor modulator is risperidone and the psychedelic is MDAI, wherein the risperidone is administered to post-treat at least 120 minutes post to the MDAI. In some embodiments, the serotonin receptor modulator is risperidone and the psychedelic is MDAI, wherein the risperidone is administered to post-treat at least 150 minutes post to the MDAI. In some embodiments, the serotonin receptor modulator is risperidone and the psychedelic is MDAI, wherein the risperidone is administered to post-treat between about 15 minutes and about 150 minutes post to the MDAI. In some embodiments, the serotonin receptor modulator is risperidone and the psychedelic is MDAI, wherein the risperidone is administered to post-treat at least 180 minutes post to the MDAI. In some embodiments, the serotonin receptor modulator is risperidone and the psychedelic is MDAI, wherein the risperidone is administered to post-treat at least 210 minutes post to the MDAI. In some embodiments, the serotonin receptor modulator is risperidone and the psychedelic is MDAI, wherein the risperidone is administered to post-treat at least 240 minutes post to the MDAI. In some embodiments, the serotonin receptor modulator is risperidone and the psychedelic is MDAI, wherein the risperidone is administered to post-treat at least 270 minutes post to the MDAI. In some embodiments, the serotonin receptor modulator is risperidone and the psychedelic is MDAI, wherein the risperidone is administered to post-treat at least 300 minutes post to the MDAI. In some embodiments, the serotonin receptor modulator is risperidone and the psychedelic is MDAI, wherein the risperidone is administered to post-treat at least 330 minutes post to the MDAI. In some embodiments, the serotonin receptor modulator is risperidone and the psychedelic is MDAI, wherein the risperidone is administered to post-treat at least 360 minutes post to the MDAI. In some preferred embodiments, the serotonin receptor modulator is risperidone and the psychedelic is MDAI, wherein risperidone is administered to post-treat between about 60 minutes and about 180 minutes post to the administration of MDAI.

In yet another aspect, also provided herein are methods of treating a disease or disorder, wherein the methods comprise administering to the subject a therapeutically effective amount of 2C-B. In some embodiments, the methods further comprise administering to the subject a serotonin receptor modulator, wherein the serotonin receptor modulator is administered at most about 3 hours post to the administration of 2C-B. In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of 2C-B. In some embodiments, the methods further comprise administering to the subject a serotonin receptor modulator, wherein the serotonin receptor modulator is administered at most about 3 hours post to the administration of 2C-B.

In yet another aspect, also provided herein are methods of treating a disease or disorder, wherein the methods comprise administering to the subject a therapeutically effective amount of 1,3-Benzodioxolyl-N-methylbutanamine (MBDB). In some embodiments, the methods further comprise administering to the subject a serotonin receptor modulator, wherein the serotonin receptor modulator is administered at most about 3 hours post to the administration of MBDB. In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of MBDB. In some embodiments, the methods further comprise administering to the subject a serotonin receptor modulator, wherein the serotonin receptor modulator is administered at most about 3 hours post to the administration of MBDB.

In yet another aspect, also provided herein are methods of treating a disease or disorder, wherein the methods comprise administering to the subject a therapeutically effective amount of 3,4-methylenedioxy-N-methylcathinone (Methylone). In some embodiments, the methods further comprise administering to the subject a serotonin receptor modulator, wherein the serotonin receptor modulator is administered at most about 3 hours post to the administration of 3,4-methylenedioxy-N-methylcathinone (Methylone). In some embodiments, the disease or disorder is depression or a disease or disorder related to depression. In some embodiments, the disease or disorder is post-traumatic stress disorder. In some embodiments, the disease or disorder is fibromyalgia. In some embodiments, the disease or disorder is a stress-related disorder. In some embodiments, the stress-related disorder is acute stress disorder (ASD), an adjustment disorder, reactive attachment disorder (RAD), and/or disinhibited social engagement disorder (DSED). In some embodiments, the disease or disorder is a musculoskeletal pain disorder including fibromyalgia, muscle pain, joint stiffness, osteoarthritis, rheumatoid arthritis, and muscle cramps. In some embodiments, the disease or disorder is a disease of women's reproductive health including premenstrual dysphoric disorder (PMDD), premenstrual syndrome (PMS), post-partum depression, and menopause.

In yet another aspect, also provided herein are methods of treating fibromyalgia or a disease or disorder related to chronic widespread pain, fatigue or hypersensitivity, wherein the methods comprise administering to the subject a therapeutically effective amount of 3,4-methylenedioxy-N-methylcathinone (Methylone). In some embodiments, the methods further comprise administering to the subject a serotonin receptor modulator, wherein the serotonin receptor modulator is administered at most about 3 hours post to the administration of 3,4-methylenedioxy-N-methylcathinone (Methylone).

In yet another aspect, provided herein are uses of a psychedelic and a serotonin receptor modulator as disclosed herein in the preparation of a medicament for treating a disease or disorder (e.g., depression or a disease or disorder related to depression), wherein the serotonin receptor modulator is released at most about 3 hours post to the release of the psychedelic.

In yet another aspect, provided herein are pharmaceutical composition comprising a psychedelic and a serotonin receptor modulator as disclosed herein for use in treating a disease or disorder (e.g., depression or a disease or disorder related to depression), wherein the serotonin receptor modulator is released at most about 3 hours post to the release of the psychedelic.

In yet another aspect, provided herein are psychedelics for use in combination with a serotonin receptor modulator in a method of treating a disease or disorder (e.g., depression or a disease or disorder related to depression), wherein the serotonin receptor modulator is released at most about 3 hours post to the release of the psychedelic.

In some embodiments, the subject is a mammal. In some embodiments, the subject is a mouse, rabbit, dog, pig, cattle, or human. In some embodiments, the subject is an infant, adult, or child. In some embodiments, the compositions described herein are administered orally, intravenously, subcutaneously, by inhalation, or by an injection. In some embodiments, the compositions described herein are administered orally. In some embodiments, the compositions described herein are administered orally via a pill, ampoule, vial, or tablet.

In some embodiments, the pharmaceutical compositions described herein (e.g., a single unit dosage form) are administered orally, intravenously, subcutaneously, by inhalation, or by an injection. In some embodiments, the pharmaceutical compositions described herein (e.g., a single unit dosage form) are administered in an injectable dosage form. In some embodiments, the pharmaceutical compositions described herein (e.g., a single unit dosage form) are administered orally. In some embodiments, the pharmaceutical compositions described herein (e.g., a single unit dosage form) are administered orally via a pill, ampoule, vial, or tablet.

In some embodiments, the compositions described herein are formulated for immediate release, modified release, sustained release, extended release, controlled release and/or delayed release. In some embodiments, the compositions described herein are solid formulations for oral administration that may be formulated for immediate and/or modified release. In some embodiments, the compositions described herein are formulated to included “modified release” coatings encompassing coatings that delay release, sustain release, extend release, prevent release, minimize release, allow for pulsed release, allow for programmed release and/or otherwise prolong the release of a drug relative to formulations lacking such coatings which release a drug relatively quickly (i.e., “immediate release” compositions).

In various embodiments herein, a composition (e.g., pharmaceutical composition, dosage form, combination or formulation) provided herein is administered at any frequency. For example, in some embodiments, a single dose is provided. In other embodiments, the composition is or is formulated to be administered to an individual in need thereof at least once a day. In other embodiments, the composition is or is formulated to be administered to an individual in need thereof once a day. In other embodiments, the composition is or is formulated to be administered to an individual in need thereof at least twice a day. In various other embodiments, the composition is or is formulated for twice daily, once daily, twice weekly, thrice weekly, or the like administration.

For administration to a subject, the compositions as disclosed herein, in some embodiments, is provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers, in some embodiments, are formulated by conventional methods and can be administered to the subject at a suitable dose. Preferably, the compositions are sterile. These compositions, in some embodiments, contain adjuvants such as preservative, emulsifying agents and dispersing agents. Prevention of the action of microorganisms, in some embodiments, is ensured by the inclusion of various antibacterial and antifungal agents.

In some embodiments, the compositions are in any suitable form, (depending upon the desired method of administration). In some embodiments, the compositions are provided in unit dosage form, provided in a sealed container, or provided as part of a kit. In some embodiments, such a kit includes instructions for use.

In some embodiments, the compositions are adapted for administration by any appropriate route, including a parenteral (e.g., subcutaneous, intramuscular, intravenous, or inhalation) route. In some embodiments, the compositions are prepared by any method known in the art of pharmacy, for example by mixing the active ingredient with the carrier(s) or excipient(s) under sterile conditions.

In some embodiments, the methods described herein are for treating a disease or disorder that is a brain disease or disorder. In some embodiments, the methods described herein are for increasing at least one of translation, transcription or secretion of neurotrophic factors. In some embodiments, the compositions provided herein have, for example, anti-addictive properties, antidepressant properties, anxiolytic properties, or a combination thereof. In some embodiments, the brain disorder is a neuropsychiatric disease. In some embodiments, the methods described herein are for treating a disease or disorder that is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder for example alcohol abuse, opiate addition, or abuse), depression, and anxiety.

In some embodiments, the brain disease or disorder is a neurodegenerative disorder, Alzheimer's disease or Parkinson's disease. In some embodiments, the brain disease or disorder is psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the methods described herein are for treating a disease or disorder that is a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, hypoxic brain injury, Chronic traumatic encephalopathy (CTE), traumatic brain injury, dementia, and addiction (e.g., substance use disorder). In some embodiments, the neurological disease is a migraine or cluster headache. In some embodiments, the neurological disease is a neurodegenerative disorder, dementia, Alzheimer's disease, or Parkinson's disease. In some embodiments, the neurological disease is dementia. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, the disease or disorder is depression or a disease or disorder related to depression.

In some embodiments, the depression is major depressive disorder, persistent depressive disorder, bipolar disorder, treatment resistant depression (TRD), postpartum depression, premenstrual dysphoric disorder, or seasonal affective disorder.

In some embodiments, the disease or disorder is an anxiety disorder. In some embodiments, the anxiety disorder is generalized anxiety disorder, post-traumatic stress disorder, obsessive-compulsive disorder, panic disorder, social anxiety disorder, phobias, or borderline personality disorder.

In some embodiments, the methods described herein are for increasing neuronal plasticity and has, for example, anti-addictive properties, antidepressant properties, anxiolytic properties, or a combination thereof. In some embodiments, decreased neuronal plasticity is associated with a neuropsychiatric disease.

In additional embodiment, the disease or disorder treated by the methods provided herein is selected from the group consisting of substance-induced mood disorder, bipolar disorder, dysthymia, major depression, major depressive disorder, post-traumatic stress disorder, generalized anxiety disorder, obsessive-compulsive disorder, panic disorder, social anxiety disorder, phobias, dementia, Parkinson's Disease, Alzheimer's Disease, vascular dementia, Lewy body dementia, frontotemporal dementia, mixed dementia, mild cognitive impairment, Creutzfeldt-Jakob disease, Wernicke-Korsakoff Syndrome, stroke, schizophrenia, paranoid personality disorder, schizoid personality disorder, schizotypal personality disorder, antisocial personality disorder, borderline personality disorder, psychosis, disorders of social isolation, disorders of disorganized behavior, disorders of aggression, disorders of agitation, disorders of compulsive behavior, disorders of excitability, disorders of hostility, disorders of self-harm, disorders of lack of restraint, cognitive disorders, delusional disorders, amnesia, mood disorders, disorders of paranoia, disorders of hallucinations, disorders of apathy, disorders of fatigue, disorders of incoherent speech, disorders of impaired motor coordination, disorders of a lack of emotional response, disorders of mental concentration, chorea, negative schizophrenia symptoms including: lack of pleasure/trouble with speech/flattening affect/social withdrawal/lack of follow through with tasks/struggles with activities of daily living, hypoxic brain injury, traumatic brain injury, Chronic traumatic encephalopathy (CTE), drug addiction, alcohol abuse, substance abuse disorder, opiate addiction, cyclothymia, persistent depressive disorder, seasonal affective disorder, hyperactivity, defiant behavior, attention deficit hyperactivity disorders, anorexia nervosa, binge eating, bulimia, anxiety disorders, suicidal behavior, separation anxiety disorder, dissociative identity disorder, dissociative amnesia, depersonalization-derealization disorder, fugue state, disruptive mood dysregulation disorder, premenstrual dysphoric disorder, antenatal depression, postpartum depression, double depression, minor depressive disorder, atypical depression, psychotic depression, bipolar I disorder, bipolar II disorder, reactive attachment disorder, disinhibited social engagement disorder, acute stress disorder, adjustment disorder, complex post-traumatic stress disorder, prolonged grief disorder, social communication disorder, autism spectrum disorder, developmental coordination disorder, Tourette syndrome, Tic disorder, dyslexia, dyscalculia, insomnia, hypersomnia, idiopathic hypersomnia, Kleine-Levin syndrome, insufficient sleep syndrome, narcolepsy, restless leg syndrome, sleep apnea, night terrors, exploding head syndrome, narcissistic personality disorder, neuropathic pain, night eating syndrome, nocturia, nosophobia, alcohol use disorder, acrophobia, early-onset dementia, ailurophobia, algophobia, anosmia, anthropophobia, aphasia, arachnophobia, arithmophobia, Asperger Syndrome, astraphobia, ataxia, ataxophobia, atelophobia, ADHD—inattentive type in adults, bipolar disorder in children, bruxism, childhood schizophrenia, chronic pain, circadian rhythm sleep disorders, claustrophobia, cocaine addiction, concussion, conversion disorder in adults, conversion disorder in children and adolescents, delirium, delusional disorder, dependent personality disorder, derealization disorder, depression in Parkinson's Disease, developmental delay, diplopia, fibromyalgia, Gaucher Disease, Guillain-Barre Syndrome, haphephobia, hoarding disorder, iatrophobia, inhalant abuse, inherited metabolic disorders, jet lag, Joubert Syndrome, Klinefelter Syndrome, learning disabilities, leukophobia, locked-in-syndrome, mania, melanophobia, microphobia, migraine, migraine aura, nicotine headache, nicotine withdrawal, oppositional defiant disorder, ophidiophobia, ornithophobia, overactive bladder, phobophobia, Prader-Willi Syndrome, primary progressive aphasia, progressive supranuclear palsy, psychosomatic disorder, Rett Syndrome, Down Syndrome, Patau Syndrome, Edwards Syndrome, schizoaffective disorder, schizophreniform disorder, self-injury, serotonin syndrome, shift work sleep disorder, sleep anxiety, sleep paralysis, sleepwalking, speech impediment, epilepsy, tinnitus, transient global amnesia, transverse myelitis, Turner syndrome, traumatic brain injury, tuberous sclerosis complex, vocal cord paralysis, Williams Syndrome, and Zellweger syndrome.

In certain embodiments, the methods described herein are for treating a disease or disorder that is a musculoskeletal pain disorder including fibromyalgia, muscle pain, joint stiffness, osteoarthritis, rheumatoid arthritis, and muscle cramps. In certain embodiments, the methods described herein are for treating a disease or disorder that is a disease of women's reproductive health including premenstrual dysphoric disorder (PMDD), premenstrual syndrome (PMS), post-partum depression, and menopause.

Described herein, in certain embodiments, are compositions comprising: a) a psychedelic; b) a serotonin receptor modulator; and c) an excipient, wherein the serotonin receptor modulator is released at most about 3 hours post to the release of the psychedelic. In some embodiments, the psychedelic is an agonist for a serotonin receptor. In some embodiments, the serotonin receptor is serotonin receptor 1B, serotonin receptor 4, serotonin receptor 6, or serotonin receptor 7. In some embodiments, the psychedelic is selected from the group consisting of psilocybin, psilocin, baeocystin, norbaeocystin, lisurgide, LSD, dimethyltryptamine, carboxamindotryptamine, ibogaine, tabernanthalog, 3,4-methylenedioxy-methamphetamine (MDMA), 1-acetyl LSD, 0-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), asymbescaline (3,4-Diethoxy-5-methoxyphenethylamine), or combinations thereof. In some embodiments, the psychedelic is LSD. In some embodiments, the LSD derivative is 1P-LSD, 1B-LSD, ETH-LAD, 1P-ETH-LAD, AL-LAD, LSZ, LSM-775, or 1-(4-Bromofuro[2,3-f][1]benzofuran-8-yl)propan-2-amine. In some embodiments, the psychedelic is mescaline. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine). In some embodiments, the psychedelic is a phenethylamine or a tryptamine. In some embodiments, the phenethylamine or the tryptamine is selected from the group consisting of 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-o-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl]dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl]dihydrogen phosphate, and 4-hydroxy-N,N,N-trimethyltryptamine, 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T, Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N-methyl-T, 5-Methoxy-N,N-tetramethylene-T, 6-Methoxy-1-methy 1-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1,2,3,4-tetrahydro-C, or alpha,N-Dimethyl-5-methoxy-T, or combinations thereof. In some embodiments, the psychedelic is psilocybin. In some embodiments, the psychedelic is 1-acetyl LSD. In some embodiments, the psychedelic is O-acetyl psilocin. In some embodiments, the serotonin receptor modulator is a serotonin receptor antagonist. In some embodiments, the serotonin receptor modulator is a serotonin receptor inverse agonist. In some embodiments, the serotonin receptor is serotonin receptor 2A. In some embodiments, the serotonin receptor modulator comprises glemanserin (MDL-11,939), eplivanserin (SR-46,349), ketanserin, ritanserin, altanserin, acepromazine, mianserin, mirtazapine, quetiapine, SB204741, SB206553, SB242084, LY272015, SB243213, Blonanserin, SB200646, RS102221, nefazodone, volinanserin (MDL-100,907), pimavanserin (ACO-103), nelotanserin, lorcaserin, flibanserin, or roluperiodone. In some embodiments, the serotonin receptor modulator is ketanserin. In some embodiments, the serotonin receptor modulator is pimavanserin. In some embodiments, the serotonin receptor modulator is eplivanserin. In some embodiments, the serotonin receptor modulator is flibanserin. In some embodiments, the serotonin receptor modulator is roluperiodone. In some embodiments, the serotonin receptor modulator is released at most about 2 hours post to the release of the psychedelic. In some embodiments, the serotonin receptor modulator is released at most about 1.5 hours post to the release of the psychedelic. In some embodiments, the serotonin receptor modulator is released at most about 1 hour post to the release of the psychedelic. In some embodiments, the serotonin receptor modulator is released at about 1 hour post to the release of the psychedelic. In some embodiments, the psychedelic is provided at a dose of about 100 milligrams (mg) to about 1 gram (g). In some embodiments, the psychedelic is provided at a dose of about 100 mg to about 800 mg. In some embodiments, the psychedelic is provided at a dose of about 100 mg to about 500 mg. In some embodiments, the psychedelic is provided at a dose of about 10 mg to about 400 mg. In some embodiments, the psychedelic is provided at a dose of about 20 mg to about 200 mg. In some embodiments, the psychedelic is provided at a dose of about 10 mg to about 100 mg. In some embodiments, the psychedelic is provided at a dose of about 10 micrograms (μg) to about 400 μg. In some embodiments, the psychedelic is provided at a dose of about 20 μg to about 200 μg. In some embodiments, the psychedelic is provided at a dose of about 10 μg to about 100 μg. In some embodiments, the serotonin receptor modulator is provided at a dose of about 10 mg to about 350 mg. In some embodiments, the serotonin receptor modulator is provided at a dose of about 20 mg to about 200 mg. In some embodiments, the serotonin receptor modulator is provided at a dose of about 10 mg to about 100 mg. In some embodiments, the psychedelic is provided at a dose of about 10 mg to about 100 mg and the serotonin receptor modulator is provided at a dose of about 10 mg to about 100 mg. In some embodiments, the psychedelic is psilocybin and the serotonin receptor modulator is ketanserin. In some embodiments, the composition is in a form of a single pill, single ampoule, or single unit dosage form.

Described herein, in certain embodiments, are methods of treating a disease or disorder in a subject in need thereof, the method comprising administering to the subject a composition comprising: a) a psychedelic; b) a serotonin receptor modulator; and c) an excipient, wherein the serotonin receptor modulator is released at most about 3 hours post to the release of the psychedelic. In some embodiments, the disease or disorder is depression or a disease or disorder related to depression. In some embodiments, the depression is major depressive disorder, persistent depressive disorder, bipolar disorder, treatment resistant depression (TRD), postpartum depression, premenstrual dysphoric disorder, or seasonal affective disorder. In some embodiments, the disease or disorder related to depression is anxiety. In some embodiments, the psychedelic is an agonist for a serotonin receptor. In some embodiments, the serotonin receptor is serotonin receptor 1B, serotonin receptor 4, serotonin receptor 6, or serotonin receptor 7. In some embodiments, the psychedelic is selected from the group consisting of psilocybin, psilocin, baeocystin, norbaeocystin, lisurgide, LSD, dimethyltryptamine, carboxamindotryptamine, ibogaine, tabemanthalog, 3,4-methylenedioxy-methamphetamine (MDMA), 1-acetyl LSD, 0-acetyl psilocin, mescaline (3,4,5-trimethoxy phenethylamine), proscaline (2-(3,5-dimethoxy-4-propoxyphenyl)ethanamine), metaescaline (2-(3-ethoxy-4,5-dimethoxyphenyl)ethanamine), allylescaline (4-Allyloxy-3,5-dimethyloxy phenylethylamine), methallylescaline (4-Methallyloxy-3,5 dimethoxyphenethylamine), asymbescaline (3,4-Diethoxy-5-methoxyphenethylamine). In some embodiments, the psychedelic is LSD. In some embodiments, the LSD derivative is 1P-LSD, 1B-LSD, ETH-LAD, 1P-ETH-LAD, AL-LAD, LSZ, LSM-775, 1-(4-Bromofuro[2,3-f] [1]benzofuran-8-yl)propan-2-amine. In some embodiments, the psychedelic is mescaline. In some embodiments, the mescaline derivative is mescaline-NBOMe, proscaline (2-(3,5-dimethoxy-4-propoxyphenyl)ethanamine), or metaescaline (2-(3-ethoxy-4,5-dimethoxyphenyl)ethanamine). In some embodiments, the psychedelic is a phenethylamine or a tryptamine. In some embodiments, the phenethylamine or the tryptamine is selected from the group consisting of 25I-NBOH, N-(2-Methoxybenzyl)-2-(3,4,5-trimethoxyphenyl)ethanamine, N-(2-hydroxybenzyl)-2,5-dimethoxy-4-iodo-phenethylamine, N-(2-hydroxybenzyl)-2,5-dimethoxy-4-chloro-phenethylamine, N-(2-hydroxybenzyl)-2,5-dimethoxy-4-bromo-phenethylamine, 4-Allyloxy-3,5-dimethyloxyphenylethylamine, N-(2-fluorobenzyl)-2,5-dimethoxy-4-iodo-phenethylamine, 2,5-dimethoxy-4-tert-butylthio-phenethylamine, 2,5-dimethoxy-4-propylthio-phenethylamine, 2,5-dimethoxy-4-propylphenethylamine, 2,5-dimethoxy-4-nitrophenethylamine, 2,5-dimethoxy-4-nitroamphetamine, 2,5-dimethoxy-4-methylphenethylamine, 2,5-dimethoxy-4-methylamphetamine, 2,5-dimethoxy-4-isopropylthio-phenethylamine, 2,5-dimethoxy-4-iodophenethylamine, 2,5-dimethoxy-4-iodoamphetamine, 2,5-dimethoxy-4-fluorophenethylamine, 2,5-dimethoxy-4-ethylthio-phenethylamine, 2,5-dimethoxy-4-ethylphenethylamine, 2,5-dimethoxy-4-cyclopropylmethylthio-phenethylamine, 2,5-dimethoxy-4-chlorophenethylamine, 2,5-dimethoxy-4-chloroamphetamine, 2,5-dimethoxy-4-bromophenethylamine, 2,5-dimethoxy-4-bromoamphetamine, 2,5-dimethoxy-4-bromo-o-ketophenethylamine, 2,5-dimethoxy-4-(2-fluoroethylthio)-phenethylamine, 2-(4-propyl-2,5-dimethoxyphenyl)-N-[(2-methoxyphenyl)methyl]ethanamine, 2-(4-methyl-2,5-dimethoxyphenyl)-N-[(2-methoxyphenyl)methyl]ethanamine, 2-(4-iodo-2,5-dimethoxyphenyl)-N-[(2-methoxyphenyl)methyl]ethanamine, 2-(4-fluoro-2,5-dimethoxyphenyl)-N-[(2-methoxyphenyl)methyl]ethanamine, 2-(4-ethyl-2,5-dimethoxyphenyl)-N-[(2-methoxyphenyl)methyl]ethanamine, 2-(4-chloro-2,5-dimethoxyphenyl)-N-[(2-methoxyphenyl)methyl]ethanamine, 2-(4-bromo-2,5-dimethoxyphenyl)-N-[(2-methoxyphenyl)methyl]ethanamine, 4-AcO-MET, 4-AcO-MALT and 4-AcO-DALT, Aeruginascin or N,N,N-trimethyl-4-phosphoryloxytryptamine, 4-Hydroxy-N,N,N-trimethyltryptamine, 5-meo-DMT, Ibogaine, [3-(2-Dimethylaminoethyl)-1H-indol-4-yl]dihydrogen phosphate, 4-hydroxytryptamine, 4-hydroxy-N,N-dimethyl-tryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl]dihydrogen phosphate, and 4-hydroxy-N,N,N-trimethyltryptamine, 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetram-ethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N, N-dimethyl-T, N-Isopropyl-4-methoxy-N-methyl-T, N-Iso-propyl-5-methoxy-N-methyl-T,5,6-Dimethoxy-N-isopropyl-N-methyl-T, 5-Methoxy-N,N-tetramethylene-T, 6-Methoxy-1-methy 1-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, 7-Methoxy-1-methyl-1,2,3,4-tetrahydro-C, or alpha,N-Dimethyl-5-methoxy-T. In some embodiments, the psychedelic is psilocybin. In some embodiments, the psychedelic is 1-acetyl LSD. In some embodiments, the psychedelic is 0-acetyl psilocin. In some embodiments, the serotonin receptor modulator is a serotonin receptor antagonist. In some embodiments, the serotonin receptor modulator is a serotonin receptor inverse agonist. In some embodiments, the serotonin receptor is serotonin receptor 2A. In some embodiments, the serotonin receptor modulator comprises ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, or lumateperone. In some embodiments, the serotonin receptor modulator is ketanserin. In some embodiments, the serotonin receptor modulator is pimavanserin. In some embodiments, the serotonin receptor modulator is eplivanserin. In some embodiments, the serotonin receptor modulator is released at most about 2 hours post to the release of the psychedelic. In some embodiments, the serotonin receptor modulator is released at most about 1.5 hours post to the release of the psychedelic. In some embodiments, the serotonin receptor modulator is released at most about 1 hour post to the release of the psychedelic. In some embodiments, the serotonin receptor modulator is released at about 1 hour post to the release of the psychedelic. In some embodiments, the psychedelic is provided at a dose of about 100 milligrams (mg) to about 1 gram (g). In some embodiments, the psychedelic is provided at a dose of about 100 mg to about 800 mg. In some embodiments, the psychedelic is provided at a dose of about 100 mg to about 500 mg. In some embodiments, the psychedelic is provided at a dose of about 10 mg to about 400 mg. In some embodiments, the psychedelic is provided at a dose of about 20 mg to about 200 mg. In some embodiments, the psychedelic is provided at a dose of about 10 mg to about 100 mg. In some embodiments, the psychedelic is provided at a dose of about 10 micrograms (μg) to about 400 μg. In some embodiments, the psychedelic is provided at a dose of about 20 μg to about 200 μg. In some embodiments, the psychedelic is provided at a dose of about 10 μg to about 100 μg. In some embodiments, the serotonin receptor modulator is provided at a dose of about 10 mg to about 350 mg. In some embodiments, the serotonin receptor modulator is provided at a dose of about 20 mg to about 200 mg. In some embodiments, the serotonin receptor modulator is provided at a dose of about 10 mg to about 100 mg. In some embodiments, the psychedelic is provided at a dose of about 10 mg to about 100 mg and the serotonin receptor modulator is provided at a dose of about 10 mg to about 100 mg. In some embodiments, the composition is in a form of a single pill, single ampoule, or single unit dosage form. In some embodiments, the psychedelic is psilocybin and the serotonin receptor modulator is ketanserin.

In some embodiments, the composition is in a form of a single pill, single ampoule, or single unit dosage form.

In some embodiments, the composition is in a single unit dosage form.

In another aspect, also provided herein are methods of treating a disease or disorder in a subject in need thereof, the method comprising administering to the subject a psychedelic and a serotonin receptor modulator, wherein the serotonin receptor is administered post to the administration of the psychedelic, and wherein the serotonin receptor modulator is administered at most about 3 hours post to the administration of the psychedelic.

In yet another aspect, also provided herein are methods of treating a disease or disorder in a subject in need thereof, the method comprising (i) administering to the subject a serotonin receptor modulator to posttreat the subject, and (ii) administering to the subject a psychedelic, and wherein the serotonin receptor modulator is administered at most about 3 hours post to the administration of the psychedelic.

In yet another aspect, also provided herein are methods of reducing the adverse effects of a psychedelic in the treatment of a disease or disorder in a subject in need thereof, the method comprising (i) administering to the subject a serotonin receptor modulator to posttreat the subject, and (ii) administering to the subject a psychedelic, and wherein the serotonin receptor modulator is administered at most about 3 hours post to the administration of the psychedelic.

In some embodiments, the disease or disorder is depression or a disease or disorder related to depression.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art. Psychedelic and 5-HT2A Antagonist Studies

### Hallucinogen-Induced Head Twitches

Mice administered the hallucinogen, LSD, were reported to respond with rapid and violent head shaking that does not occur in normal mice. See, Keller and Umbreit, Science, 1956, 124: 723). This response was found to be remarkably consistent when scored by different observers across laboratories. The head-shake response is elicited by a wide variety of known hallucinogens such as LSD, psilocybin, psilocin, N,N-dimethyltryptamine (DMT), and mescaline as well as serotonin-releasing agents and direct 5-HT2 agonists. See, Canal and Morgan, Drug Test Anal., 2012, 4, 556-576 2012. 2,5-dimethoxy-4-iodoamphetamine (DOI) has also been reported to elicit head shakes in rats (see Amt and Hyttel Eur. J. Pharmacol., 1989, 161:45; also see Kennett et al., Br. J. Pharmacol., 1994, 111: 797-802) and head-twitches in mice (see Darmani et al., Pharmacol. Biochem. Behav., 1990, 36: 901-606), both of which were blocked by administration of the fairly selective 5-HT2A antagonist ketanserin. Later studies have confirmed 5-HT2A receptors are the primary, direct mediators of the response and that the headshake response in rats is essentially the same as head-twitches in mice, at least in regards to similarity in appearance and 5-HT2A receptor dependence. See, Canal and Morgan, Drug Test Anal., 2012, 4, 556-576 2012. The head twitch and head shake response in mice and rats have therefore been widely used to explore the effect of treatments on 5-HT2A receptors in vivo.

### Animals for all Studies

Male C57BL/6J mice (approximately 25 g) was group housed in stock room B20D of the Saretius animal at the University of Reading. Animals was maintained under a 12 h light/dark cycle, at 23° C. with humidity controlled

### Example 1: Effect of Psilocybin and LSD on Head Twitches Responses in Mice

Psilocybin was formulated in saline at a concentration of 0.2 mg/mL to give a dose of 1 mg/kg when administered i.p. in a 5 mL/kg dosing volume. Lysergic acid diethylamine tartrate (LSD tartrate) was formulated in saline at a concentration of 0.02 and 0.063 mg free base/mL to give doses of 0.1 and 0.32 mg/kg when administered i.p. in a 5 mL/kg dosing volume. LSD was supplied as a tartrate salt with 68.3% free base content.

### Procedure

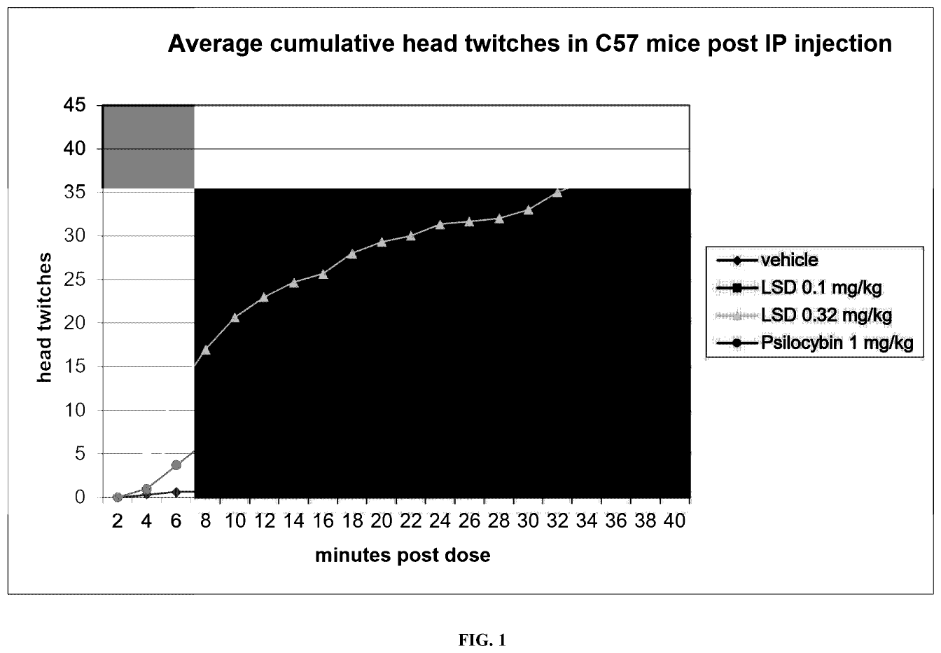

At T=−1 h, Mice were individually housed into transparent test cages with bedding removed and left to habituate. At T=0 h, groups of 3 mice were dosed intraperitoneally with vehicle (saline), psilocybin (1 mg/kg) or LSD (0.1 or 0.32 mg/kg). Following dosing, mice were replaced into their test cages and head-shake responses continuously scored for 40 min. Head twitches per minutes post dose was illustrated in FIG. 1. Test condition groups of Example 1 are summarized in Table 1.

TABLE 1
Synopsis of head shake test pilot schedule
for psilocybin and LSD in mice

| | | | T = 0-40 min |
|---|---|---|---|
| | 1 h pre-test | T = 0 pre-test | Assess no of |
| Group | Place in cage | Administered i.p. | head shakes 40 |
| (n) | to habituate | (5 mL/kg saline) | min per mouse |
| 3 | Yes | Vehicle | Yes |
| 3 | Yes | Psilocybin 1 mg/kg | Yes |
| 3 | Yes | LSD 0.1 mg/kg | Yes |
| 3 | Yes | LSD 0.32 mg/kg | Yes |

### Example 2: Comparison of LSD Induced Head Twitches in Mice with and without Administration of Volinanserin after T=5 Minutes

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Volinanserin HCl (91.1% free base content) was dissolved in saline at concentrations of 0.2 mg/ml to give a dose of 1 mg/kg when administered intravenously (i.v.) in a 5 mL/kg dosing volume.

### Procedure

At T=−60 min, C57BL/6J mice were individually housed into transparent test cages with bedding removed and left to habituate. At T=−4 min, mice were placed in a Heated cage at 40° C.

At T=0 hr, groups of 3 mice were dosed intraperitoneally with psilocybin (1 mg/kg) or LSD (0.32 mg/kg). Following dosing, mice were replaced into the heated cages and head-twitch responses continuously scored for 5 min. After 5 min, mice were intravenously dosed via the lateral tail vein with either vehicle or volinanserin 1 mg/kg in 5 mL/kg dosing volumes.

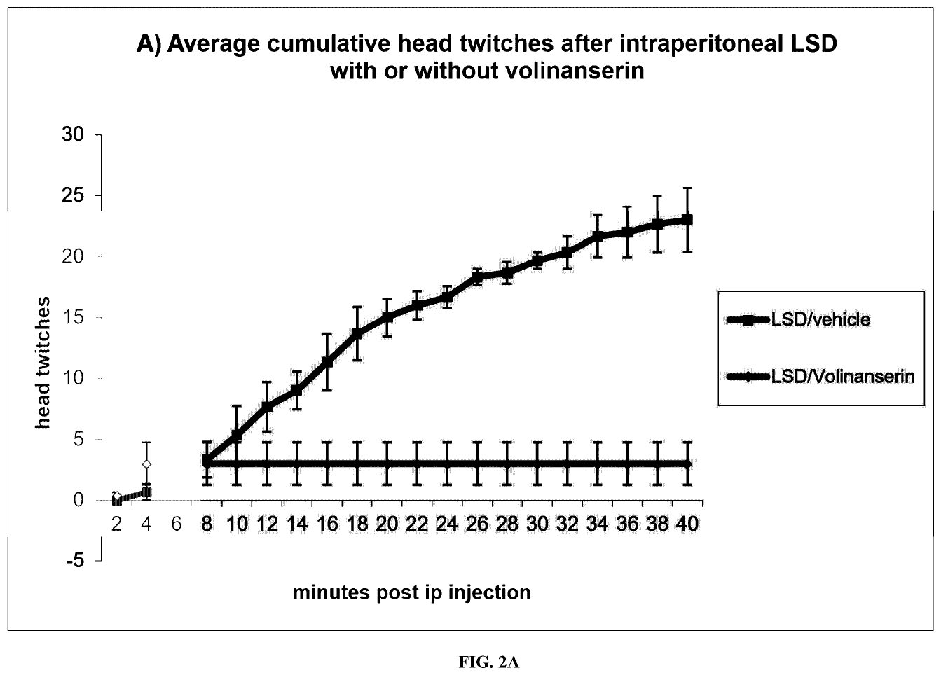

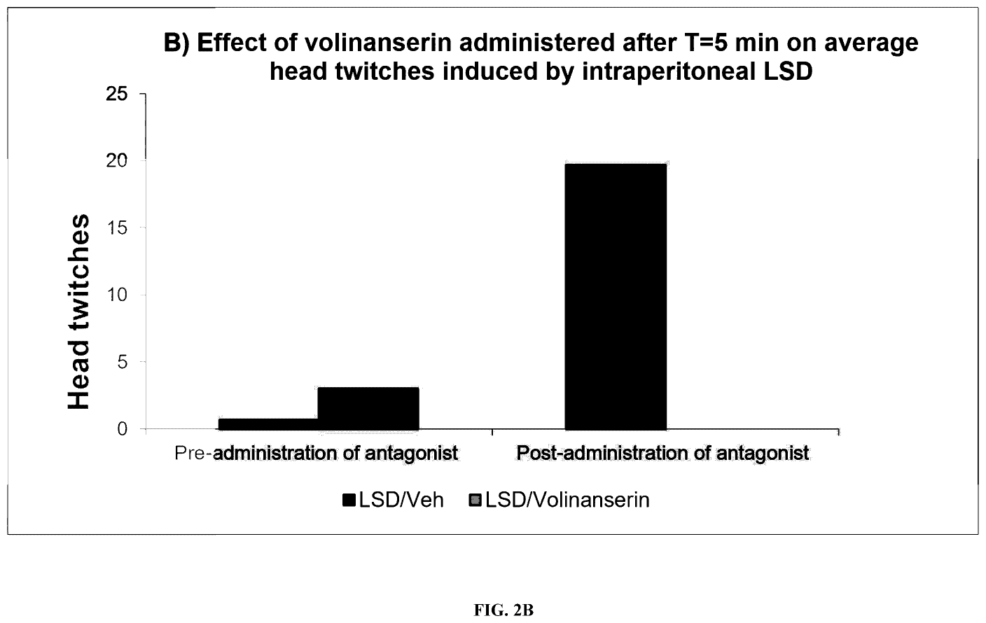

Mice were replaced into the observation cages and head twitch behavior were monitored until 40 min after agonist dosing.
Mice were dosed intraperitoneally with LSD (0.32 mg/kg) and head-twitch responses were continuously scored for 5 min. After 5 min, mice were intravenously dosed with either vehicle (saline) or volinanserin (1 mg/kg). Cumulative head-twitches were measured every 2 minutes until 40 minutes after agonist dosing. As shown in FIG. 2, administration of volinanserin at this time point completely suppressed the head-twitch response. FIG. 2A shows a graph, FIG. 2B shows a bar chart illustrating head twitches occurring before and after control or antagonist administration. Test condition groups of Example 2 are summarized in Table 2.

TABLE 2
Synopsis of mouse twitch test pilot schedule

| | 60 min pretest | | | | T = 0-40 min |
|---|---|---|---|---|---|
| | Place in | 5 min pretest | T = 0 pretest | T = 5 min | Assess number of |
| Group | test cage to | Place in | Treatment i.p. | Treatment i.v. | head shakes 40 |
| (n) | habituate | heated cage | (5 mL/kg saline) | (2 mL/kg saline) | min per mouse |
| 3 | Yes | Yes | LSD 0.32 mg/kg | Vehicle | Yes |
| 3 | yes | yes | LSD 0.32 mg/kg | Volinanserin 1 mg/kg | Yes |

### Example 3: Comparison of LSD Induced Head Twitches in Mice with and without Administration of Volinanserin after T=10 Minutes

### Formulation

Psilocybin was formulated in saline at a concentration of 0.2 mg/mL to give a dose of 1 mg/kg when administered i.p. in a 5 mL/kg dosing volume. Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume.

Volinanserin HCl (91.1% free base content) was dissolved in Vehicle 1 (saline) at concentrations of 0.2 mg/ml to give a dose of 1 mg/kg when administered i.v. in a 5 mL/kg dosing volume.

### Procedure

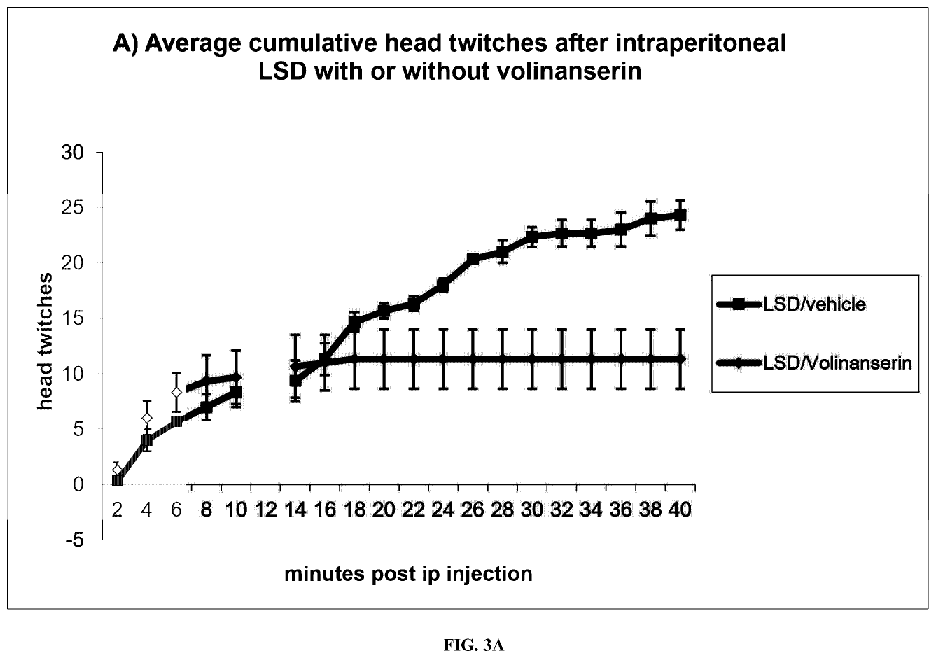

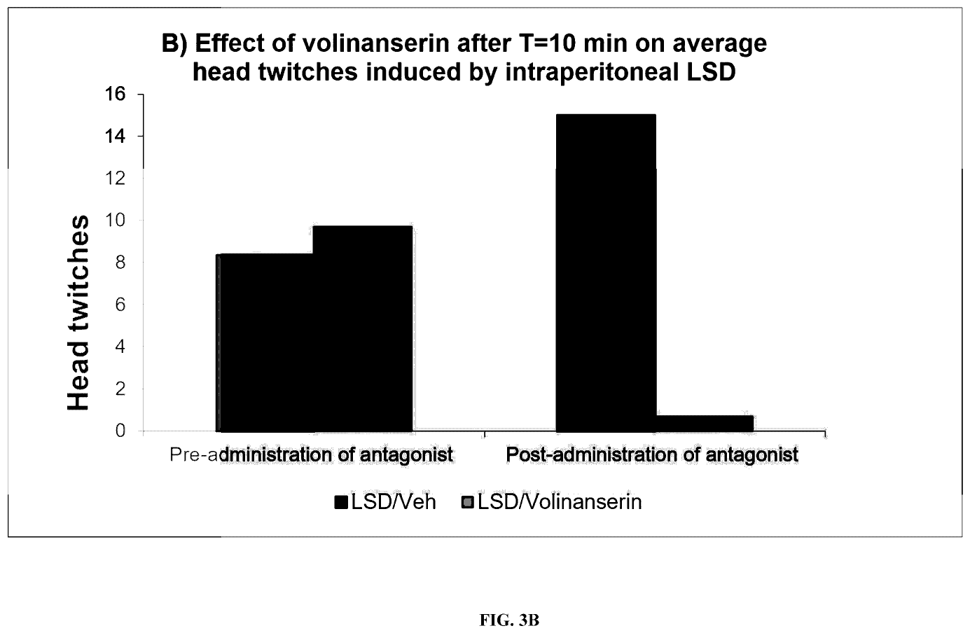

At T=−60 min, C57BL/6J mice were individually housed into transparent observation cages with bedding removed and left to habituate. At T=0 h, groups of 3 mice were dosed intraperitoneally with LSD (0.32 mg/kg). Following dosing, mice were placed into heated cages at 40° C. and head-twitch responses continuously scored for 10 min. After 10 min, mice were intravenously dosed via the lateral tail vein with either vehicle 1 (saline) or volinanserin 1 mg/kg in 5 mL/kg dosing volumes. Mice were replaced into the observation cages and head twitch behavior were monitored until 40 min after agonist dosing. Cumulative head-twitch behavior was measured every 2 minutes until 40 minutes after agonist dosing. As shown in FIG. 3, administration of volinanserin at this time point completely suppressed the head-twitch response. FIG. 3A shows a graph, FIG. 3B shows a bar chart showing head twitches occurring before and after control or antagonist administration. Test condition groups of Example 3 are summarized in Table 3.

TABLE 3
Synopsis of mouse twitch test pilot schedule

| | 60 min pre-test | | | T = 10 min | T = 0-40 min |
|---|---|---|---|---|---|
| | Place in | T = 0 pretest | | Treatment i.v. | Assess number of |
| Grp | test cage to | Treatment i.p. | Place in | (2 mL/kg) and | head shakes 40 |
| (n) | habituate | (5 mL/kg saline) | heated cage | replace in test cage | min per mouse |
| 3 | Yes | LSD 0.32 mg/kg | Yes | Vehicle 1 | Yes |
| 3 | yes | LSD 0.32 mg/kg | Yes | Volinanserin 1 mg/kg | Yes |

### Example 4: Comparison of Psilocybin Induced Head Twitches in Mice with and without Administration of Volinanserin after T=8 Minutes

### Formulation

Psilocybin was formulated in saline at a concentration of 0.4 mg/mL to give a dose of 2 mg/kg when administered i.p. in a 5 mL/kg dosing volume. Volinanserin HCl (91.1% free base content) was dissolved in Vehicle 1 (saline) at concentrations of 0.2 mg/ml to give a dose of 1 mg/kg when administered i.v. in a 5 mL/kg dosing volume.

### Procedure

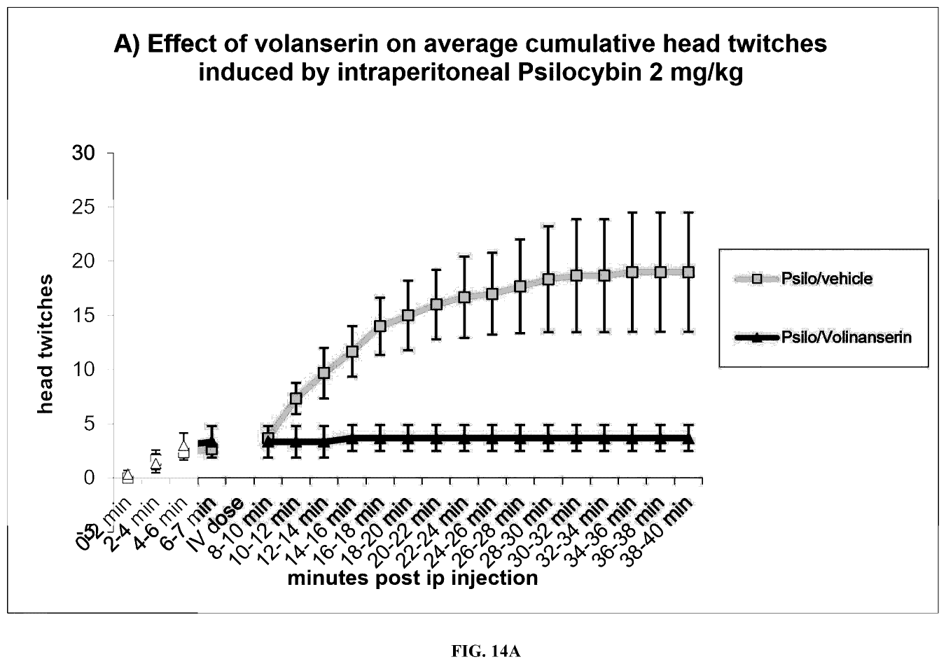

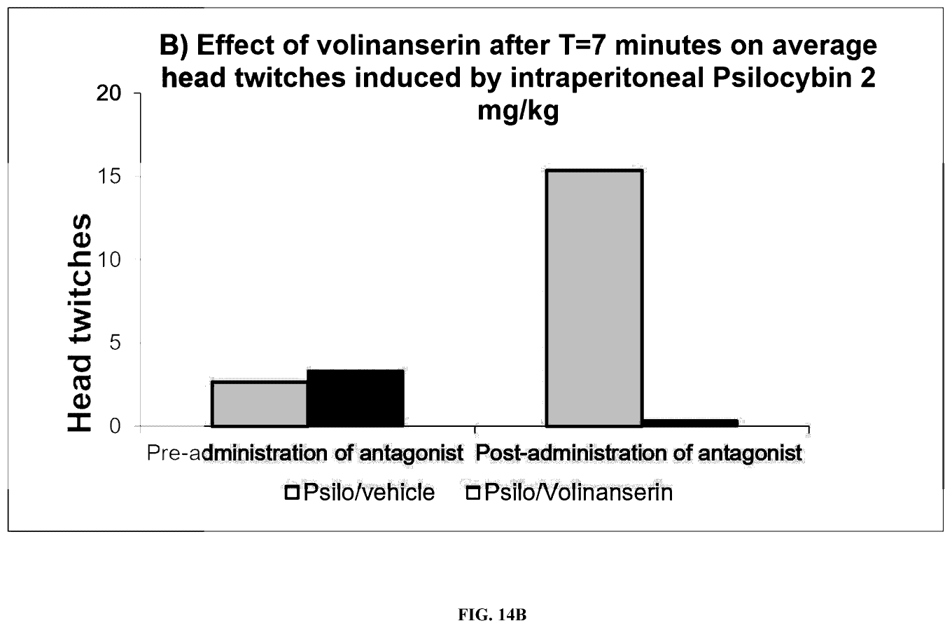

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (saline) or volinanserin 1 mg/kg in 5 mL/kg dosing volumes. Mice were replaced into the observation cages and head twitch behavior were monitored until 40 min after agonist dosing. Cumulative head-twitches were measured every 2 minutes until 40 minutes after agonist dosing. As shown in FIG. 14, administration of volinanserin at this time point completely suppressed the head-twitch response. FIG. 14A shows a graph, FIG. 14B shows a bar chart illustrating that head twitches occurring before and after control or antagonist administration. Test condition groups of Example 4 are summarized in Table 4.

TABLE 4
Synopsis of mouse twitch test pilot schedule

| | 60 min pre-test | | | T = 7 min | T = 0-40 min |
|---|---|---|---|---|---|
| | Place in | T = −3 min | T = 0 pretest | Treatment i.v. | Assess number of |
| Grp | test cage to | Place in | Treatment i.p. | (5 mL/kg) and | head shakes 40 |
| (n) | habituate | heated cage | (5 mL/kg saline) | replace in test cage | min per mouse |
| 3 | yes | Yes | Psilocybin 2 mg/kg | Vehicle 1 | Yes |
| 3 | yes | Yes | Psilocybin 2 mg/kg | Volinanserin 1 mg/kg | Yes |

### Example 5A: Effect of Intravenous Administration of Volinanserin on LSD-Induced Head Twitches in Mice

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Volinanserin HCl (91.1% free base content) was dissolved in Vehicle 1 (saline) at concentrations of 0.2 mg/ml to give a dose of 1 mg/kg when administered i.v. in a 5 mL/kg dosing volume.

### Procedure

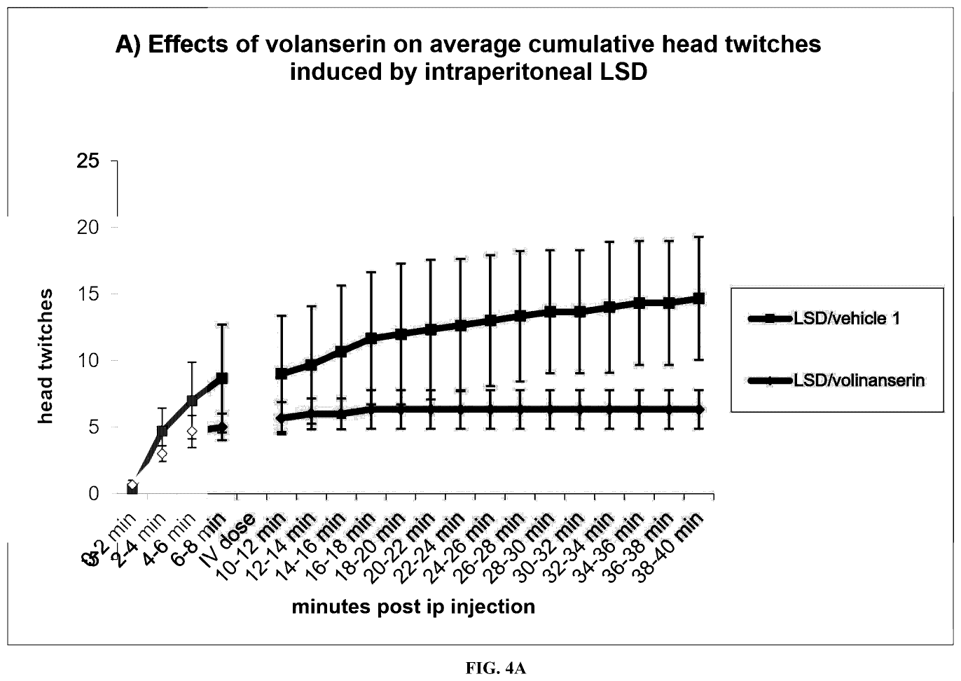

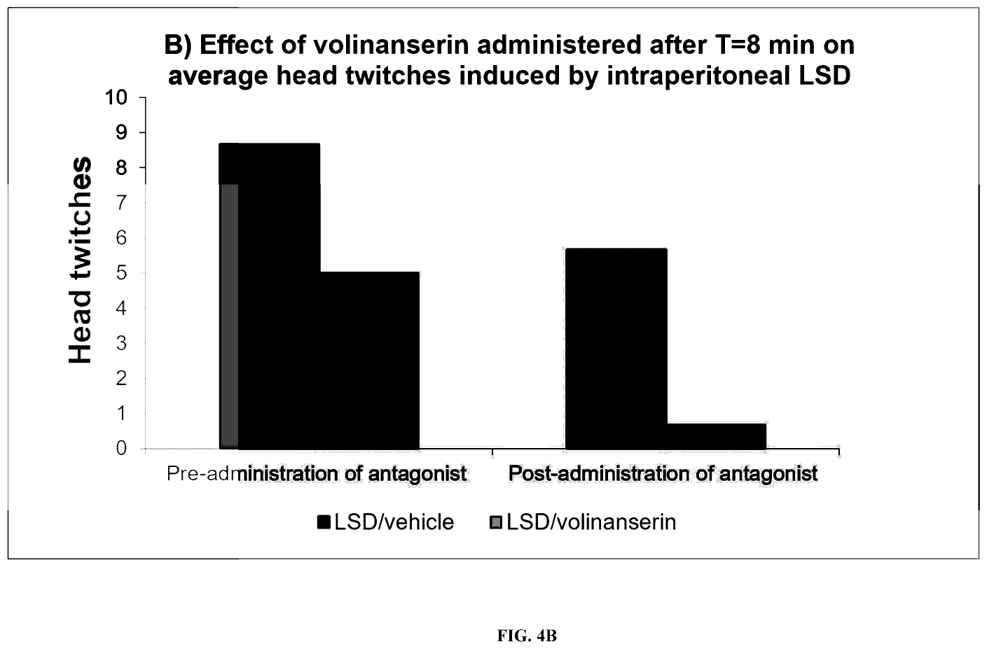

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 1 (saline) or volinanserin 1 mg/kg in 5 mL/kg dosing volumes. Mice were replaced into the observation cages and head twitch behavior were monitored until 40 min after agonist dosing. Cumulative head-twitch behavior was measured every 2 minutes until 40 minutes after agonist dosing. As shown in FIG. 4, administration of volinanserin at this time point completely suppressed the head-twitch response. FIG. 4A shows a graph, FIG. 4B shows a bar chart illustrating head twitches occurring before and after control or antagonist administration. Test condition groups of Example 5A are summarized in Table 5.

TABLE 5
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | LSD 0.32 mg/kg | Vehicle 1 | Yes |
| 3 | yes | yes | LSD 0.32 mg/kg | Volinanserin 1 mg/kg | Yes |

### Example 5B: Effect of Intravenous Administration of Ketanserin on LSD-Induced Head Twitches in Mice

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Ketanserin Tartrate (74.7% free base content) was dissolved in Vehicle 2 (DMSO: Cremophor EL: Hydroxypropyl-o-cyclodextrin (20% in water) [10:10:80]) at concentrations of 0.4 mg/ml to give a dose of 2 mg/kg when administered i.v. in a 5 mL/kg dosing volume.

### Procedure

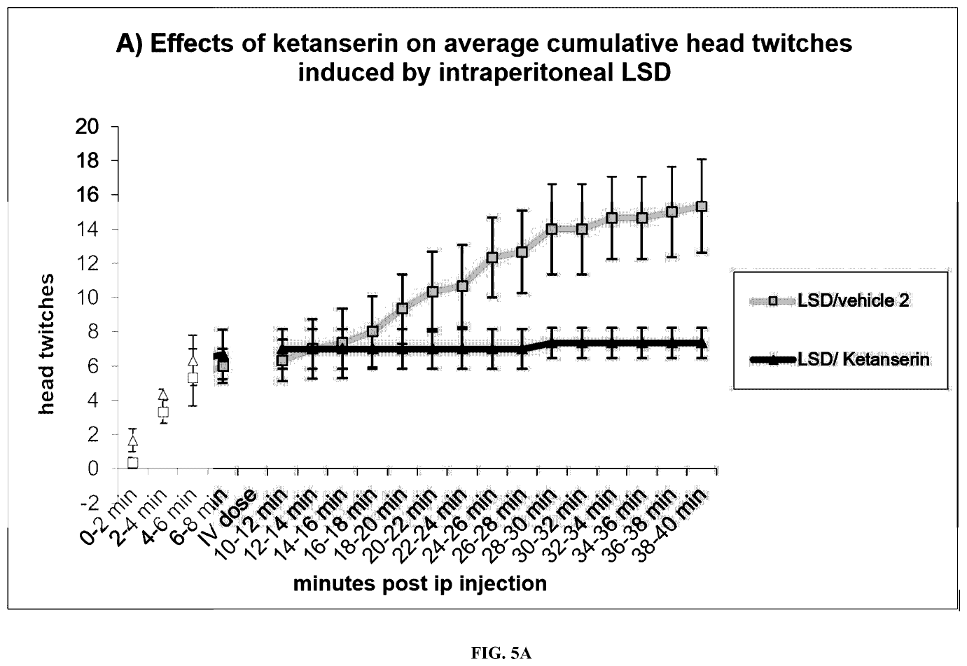

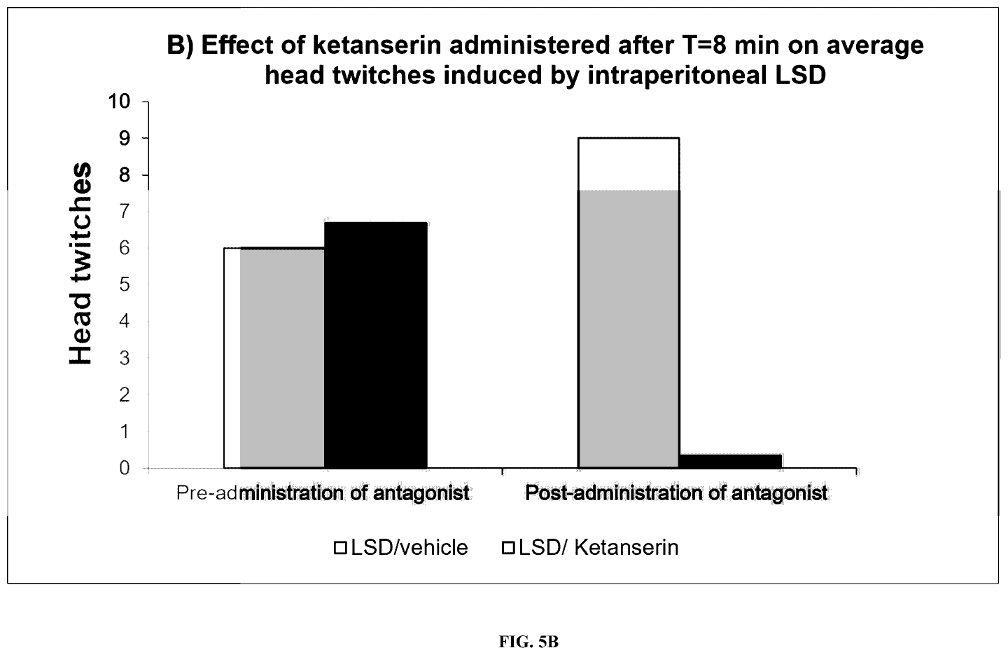

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with Vehicle 2 (DMSO:Cremophor:HPCD) or ketanserin (2 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 5, administration of ketanserin completely suppressed the head-twitch response. FIG. 5A shows a graph, FIG. 5B shows a bar chart showing head twitches occurring before and after control or antagonist administration. Test condition groups of Example 5B are summarized in Table 6.

TABLE 6
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | yes | yes | LSD 0.32 mg/kg | Vehicle 2 | Yes |
| 3 | yes | yes | LSD 0.32 mg/kg | Ketanserin 2 mg/kg | Yes |

### Example 6: Effect of Intravenous Administration of Eplivanserin and Pimavanserin on LSD-Induced Head Twitches in Mice

### Animal

9 Male C57BL/6J mice (approximately 25 g) were group housed in stock room B20D of the Saretius animal at the University of Reading. Animals were maintained under a 12 h light/dark cycle, at 23° C. with humidity controlled according to Home Office regulations.

### Formulation Checks

Pimavanserin Tartrate (85.07% free base content) will be assessed in up to 4 different formulations to identify a vehicle suitable for intravenous (i.v.) administration in which it forms a solution at a concentrations of 0.2 mg free base equivalents/ml to give a dose of 1 mg/kg when administered i.v. in a 5 mL/kg dosing volume.

Eplivanserin will be assessed in up to 4 different formulations to identify a vehicle suitable for i.v. administration in which it forms a solution at a concentrations of 0.2 mg free base equivalents/ml to give a dose of 1 mg/kg when administered i.v. in a 5 mL/kg dosing volume.

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Eplivanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Pimavanserin Tartrate (85.07% free base content) was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

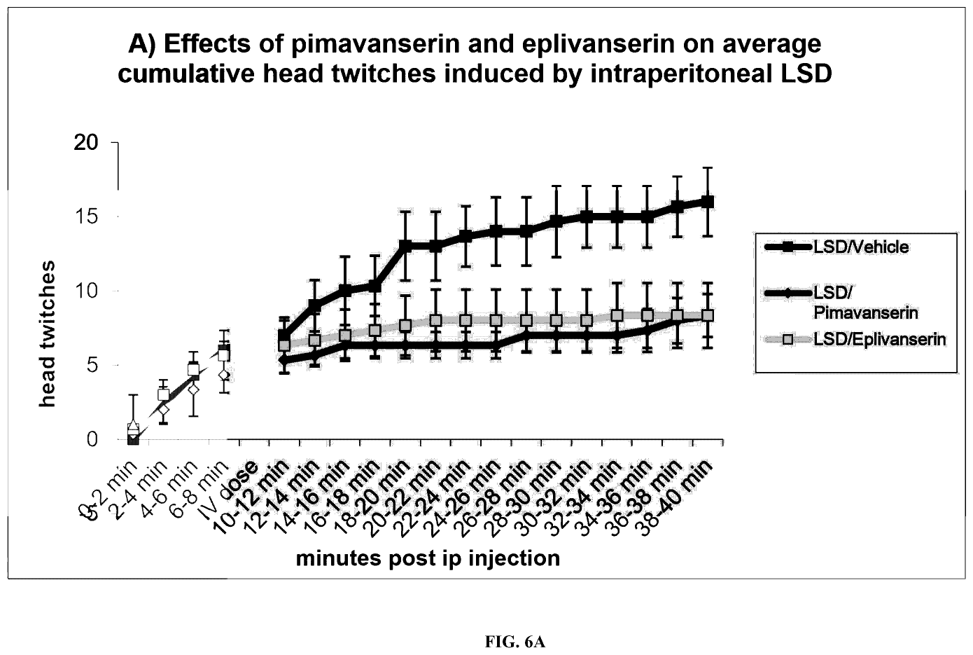

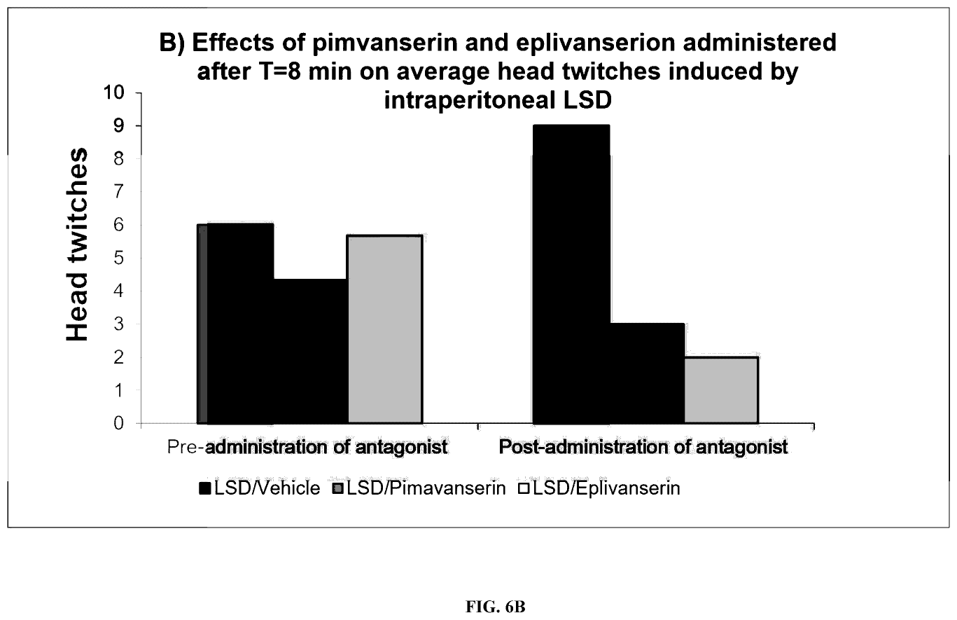

At T=−60 min, C57BL/6J mice were individually housed into transparent observation cages with bedding removed and left to habituate. At T=−2 min, mice were placed into heated cages at 40° C. At T=0 h, groups of 3 mice will be dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 1 (DMSO:HPCD [10:90]) or eplivanserin 1 mg/kg or pimavanserin (1 mg/kg) in 5 mL/kg dosing volumes. Mice were then replaced into the observation cages and head twitch behavior monitored until 40 min after agonist dosing. Cumulative head-twitch behavior was measured every 2 minutes until 40 minutes after agonist dosing. As shown in FIG. 6, administration of eplivanserin and pimavanserin partially suppressed the head-twitch response. FIG. 6A shows a graph, FIG. 6B shows a bar chart illustrating head twitches occurring before and after control or antagonist administration. Test condition groups of Example 6 are summarized in Table 7.

TABLE 7
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | LSD 0.32 mg/kg | Vehicle 1 | Yes |
| 3 | yes | yes | LSD 0.32 mg/kg | Eplivanserin 1 mg/kg | Yes |
| 3 | yes | yes | LSD 0.32 mg/kg | Pimavanserin 1 mg/kg | Yes |

### Example 7: Effect of Intravenous Administration of Eplivanserin and Pimavanserin on Psilocybin Induced Head Twitches in Mice

### Formulation

Psilocybin was formulated in saline at a concentration of 0.4 mg/mL to give a dose of 2 mg/kg when administered i.p. in a 5 mL/kg dosing volume. Eplivanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Pimavanserin Tartrate (85.07% free base content) was dissolved in Vehicle 1 (DMSO:HPCD=10:90) at concentrations of 0.2 mg free base equivalents/ml to give a dose of 1 mg/kg when administered i.v. in a 5 mL/kg dosing volume.

### Procedure

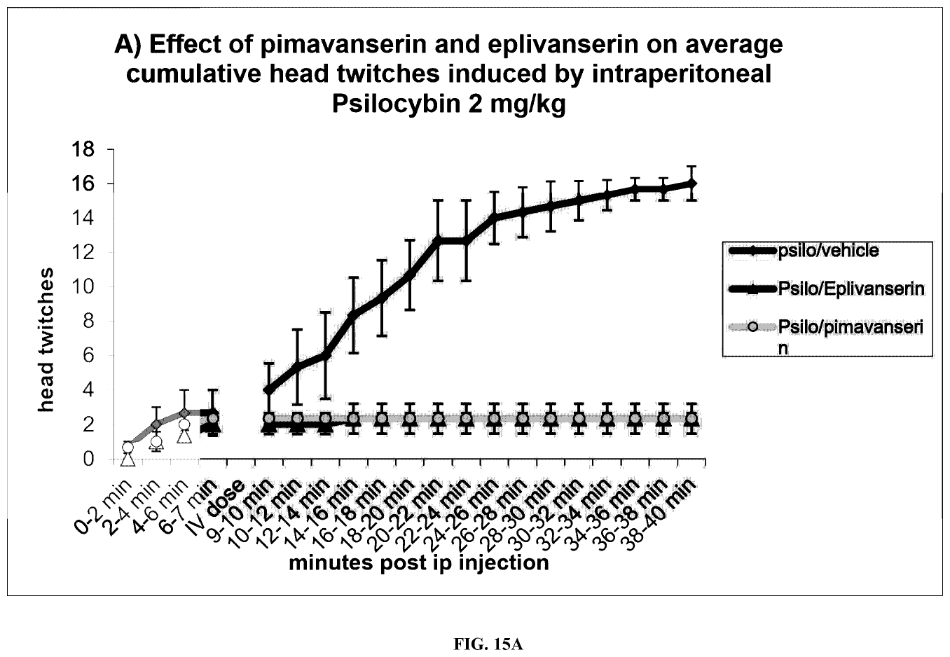

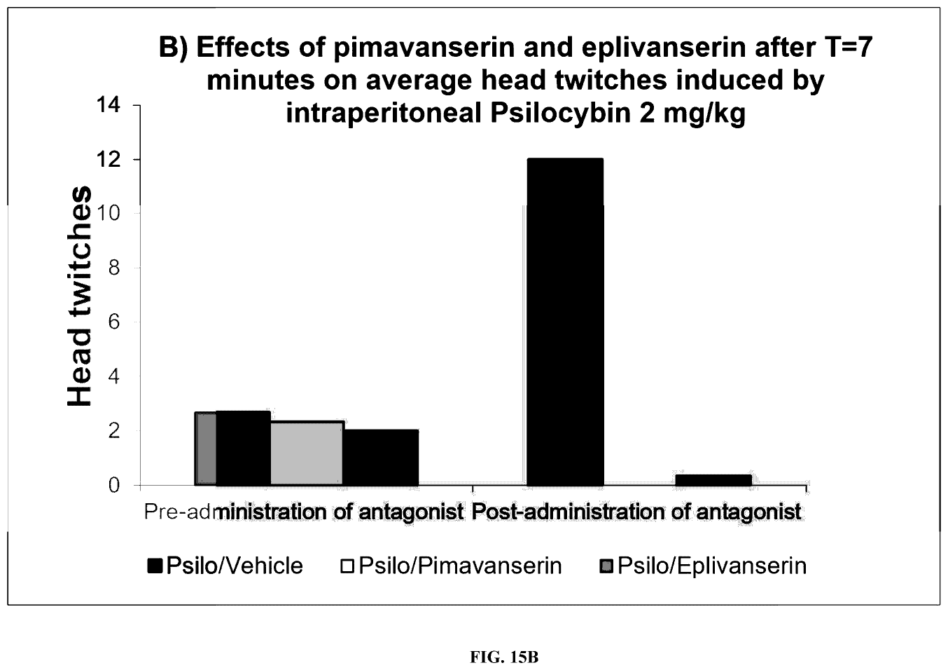

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (DMSO:HPCD [10:90]) or eplivanserin 1 mg/kg or pimavanserin (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 15, administration of eplivanserin and pimavanserin completely suppressed the head-twitch response. FIG. 15A shows a graph, FIG. 15B shows a bar chart showing head twitches occurring before and after control or antagonist administration. Test condition groups of Example 7 are summarized in Table 8.

TABLE 8
Synopsis of mouse twitch test pilot schedule

| | 60 min pre-test | | | T = 7 min | T = 0-40 min |
|---|---|---|---|---|---|
| | Place in | T = −3 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | Psilocybin 2 mg/kg | Vehicle 1 | Yes |
| 3 | yes | yes | Psilocybin 2 mg/kg | Eplivanserin 1 mg/kg | Yes |
| 3 | yes | yes | Psilocybin 2 mg/kg | Pimavanserin 1 mg/kg | Yes |

### Example 8: Effect of Intravenous Administration of Risperidone and Pruvanserin on LSD-Induced Head Twitches in Mice

### Formulation Checks

Pruvanserin was assessed in up to 4 different formulations to identify a vehicle suitable for i.v. administration in which it forms a solution at a concentrations of 0.2 mg free base equivalents/ml to give a dose of 1 mg/kg when administered i.v. in a 5 mL/kg dosing volume.

Risperidone was assessed in up to 4 different formulations to identify a vehicle suitable for i.v. administration in which it forms a solution at a concentrations of 0.2 mg free base equivalents/ml to give a dose of 1 mg/kg when administered i.v. in a 5 mL/kg dosing volume.

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Risperidone was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Pruvanserin (free base) was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

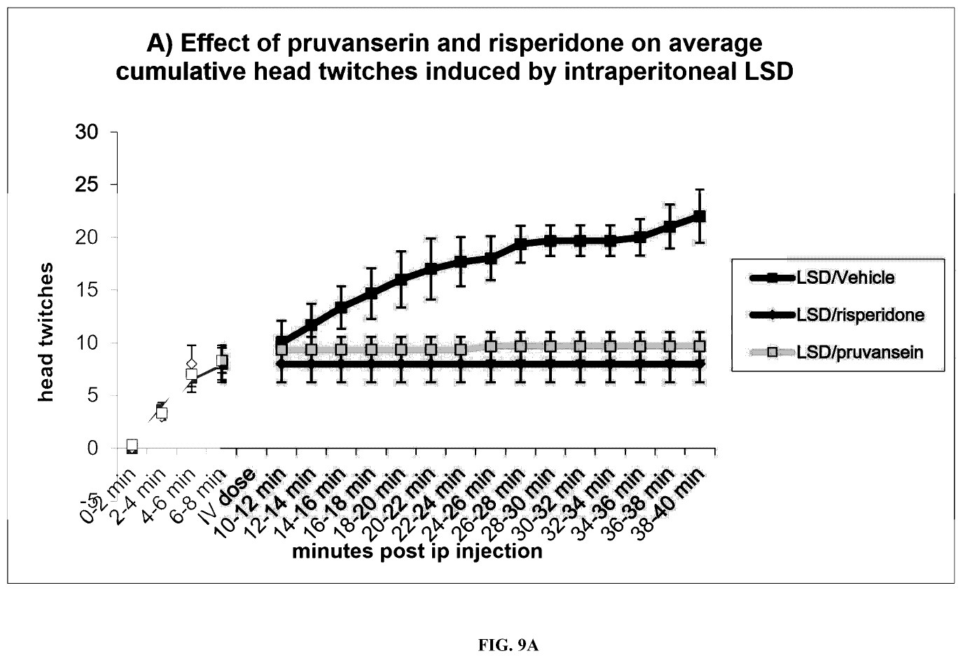

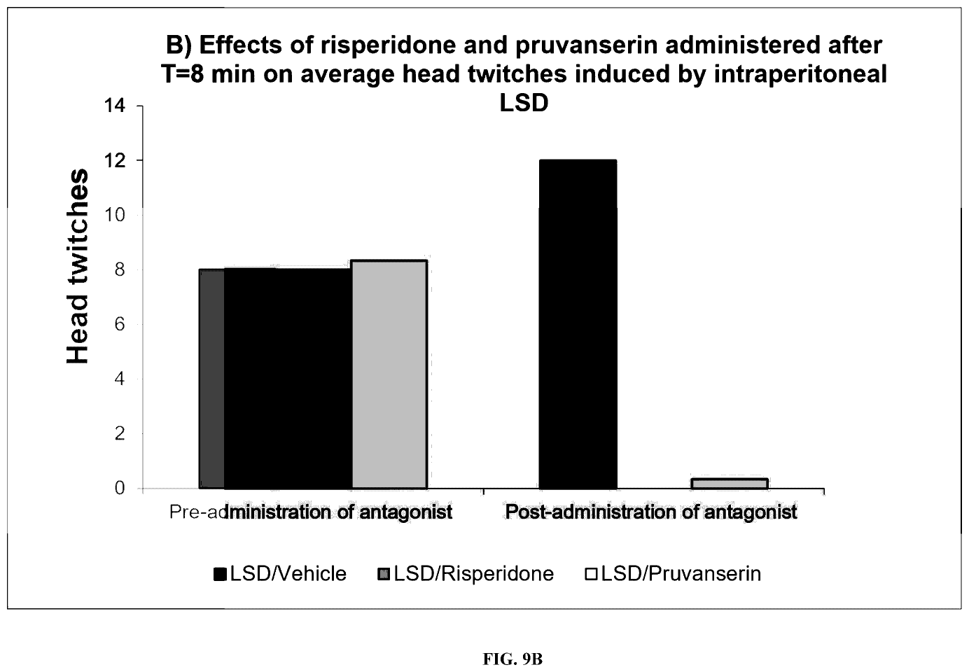

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 1 (DMSO:HPCD [10:90]) or risperidone 1 mg/kg or pruvanserin (1 mg/kg) in 5 mL/kg dosing volumes. Mice were replaced into the observation cages and head twitch behavior was monitored until 40 min after agonist dosing. As shown in FIG. 9, administration of risperidone and pruvanserin completely suppressed the head-twitch response. FIG. 9A shows a graph, FIG. 9B shows a bar chart showing head twitches occurring before and after control or antagonist administration. Test condition groups of Example 8 are summarized in Table 9.

TABLE 9
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Grp | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | LSD 0.32 mg/kg | Vehicle 1 | Yes |
| 3 | yes | yes | LSD 0.32 mg/kg | Risperidone 1 mg/kg | Yes |
| 3 | yes | yes | LSD 0.32 mg/kg | Pruvanserin 1 mg/kg | Yes |

### Example 9: Effect of Intravenous Administration of Risperidone and Pruvanserin on Psilocybin Induced Head Twitches in Mice

### Formulation

Psilocybin was formulated in saline at a concentration of 0.4 mg/mL to give a dose of 2 mg/kg when administered i.p. in a 5 mL/kg dosing volume. Risperidone was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Pruvanserin Tartrate (85.07% free base content) was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

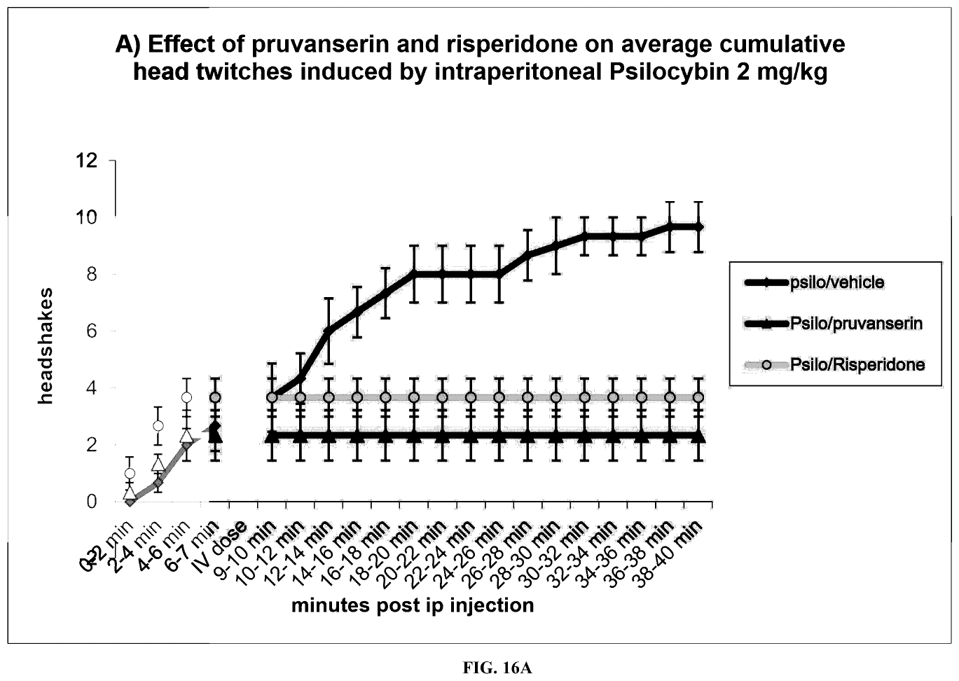

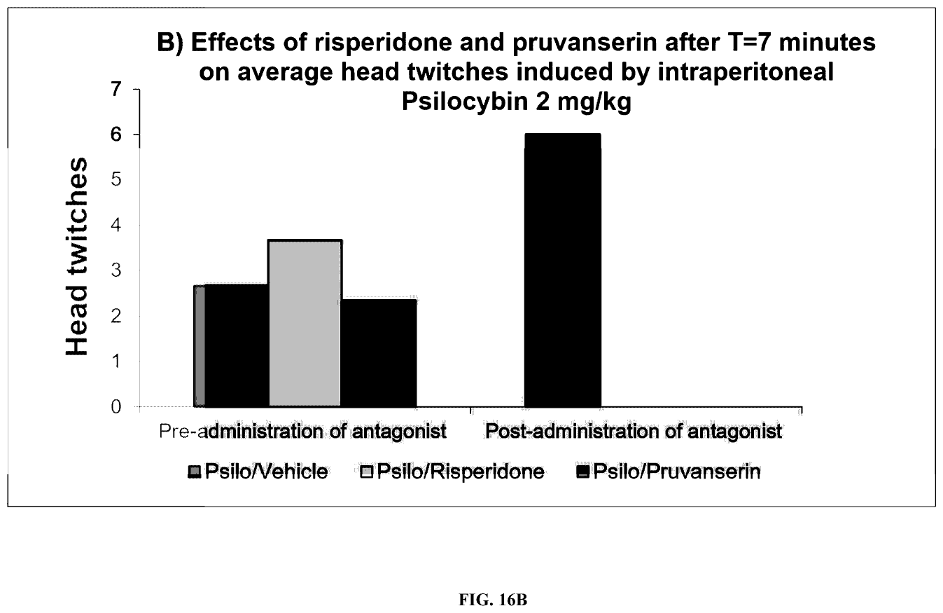

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (DMSO:HPCD [10:90]) or risperidone 1 mg/kg or pruvanserin (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 16, administration of pruvanserin and risperidone completely suppressed the head-twitch response. FIG. 16A shows a graph, FIG. 16B shows a bar chart showing head twitches occurring before and after control or antagonist administration. Test condition groups of Example 9 are summarized in Table 10.

TABLE 10
Synopsis of mouse twitch test pilot schedule

| | 60 min pre-test | | | T = 7 min | T = 0-40 min |
|---|---|---|---|---|---|
| | Place in | T = −3 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | Psilocybin 2 mg/kg | Vehicle 1 | Yes |
| 3 | yes | yes | Psilocybin 2 mg/kg | Risperidone 1 mg/kg | Yes |
| 3 | yes | yes | Psilocybin 2 mg/kg | Pruvanserin 1 mg/kg | Yes |

### Example 10: Effect of Intravenous Administration of Ritanserin, Nelotanserin and Olanzapine on LSD-Induced Head Twitches in Mice

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Ritanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Nelotanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Olanzapine was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

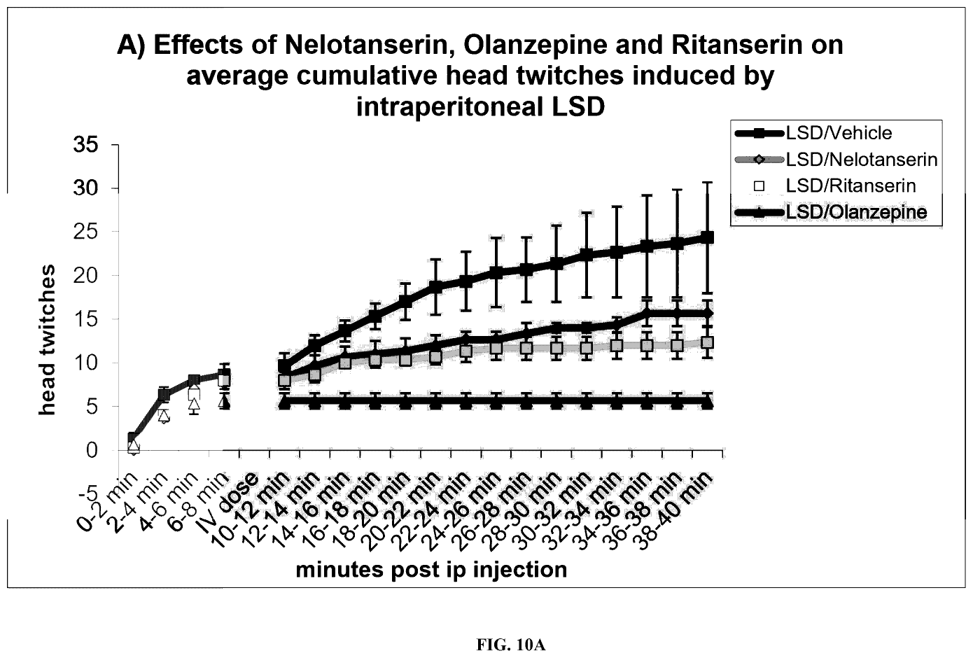

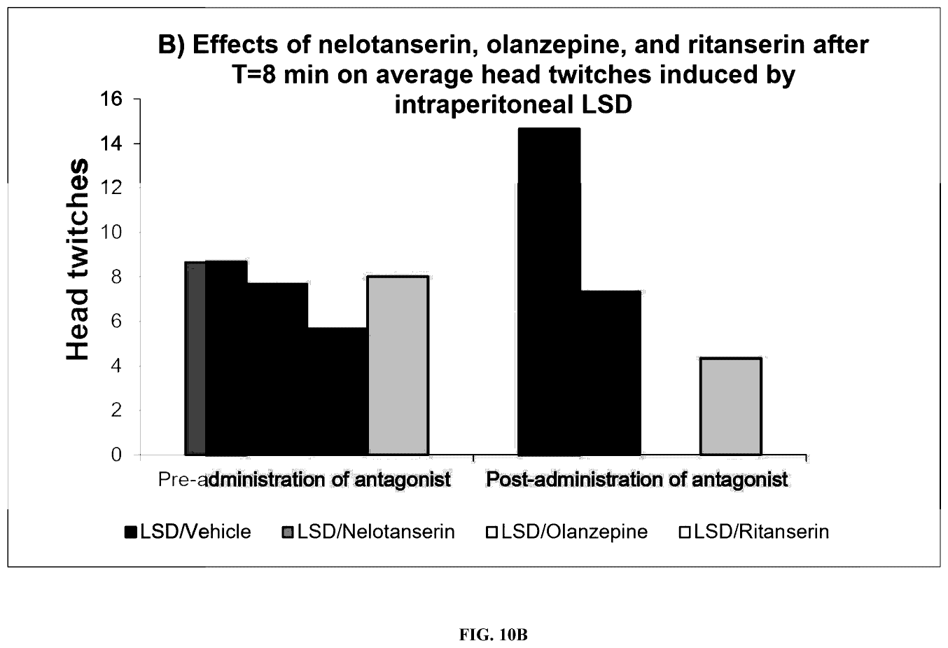

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 1 (DMSO:HPCD [10:90]) or ritanserin (1 mg/kg) or nelotanserin (1 mg/kg) or olanzapine (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 10, administration of olanzapine completely suppressed the head-twitch response, and administration of ritanserin and nelotanserin partially suppressed the head-twitch response. FIG. 10A shows a graph, FIG. 10B shows a bar chart showing head twitches occurring before and after control or antagonist administration. Test condition groups of Example 10 are summarized in Table 11.

TABLE 11
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | LSD 0.32 mg/kg | Vehicle 1 | Yes |
| 3 | yes | yes | LSD 0.32 mg/kg | Ritanserin 1 mg/kg | Yes |
| 3 | yes | yes | LSD 0.32 mg/kg | Nelotanserin 1 mg/kg | Yes |
| 3 | yes | yes | LSD 0.32 mg/kg | Olanzapine 1 mg/kg | Yes |

### Example 11: Effect of Intravenous Administration of Ritanserin, Nelotanserin and Olanzapine on Psilocybin Induced Head Twitches in Mice

### Formulation

Psilocybin was formulated in saline at a concentration of 0.4 mg/mL to give a dose of 2 mg/kg when administered i.p. in a 5 mL/kg dosing volume. Ritanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Nelotanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume. Olanzapine was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

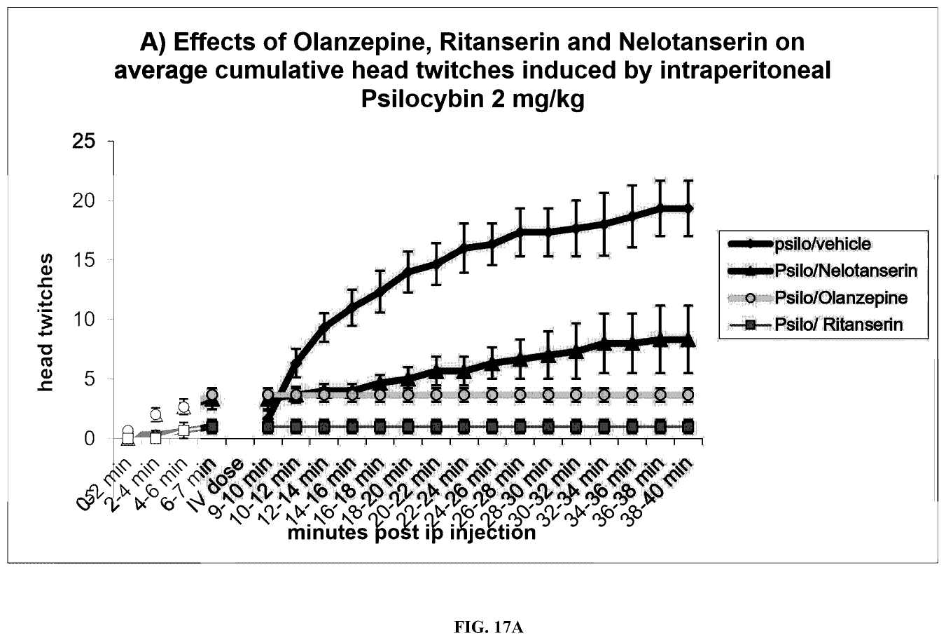

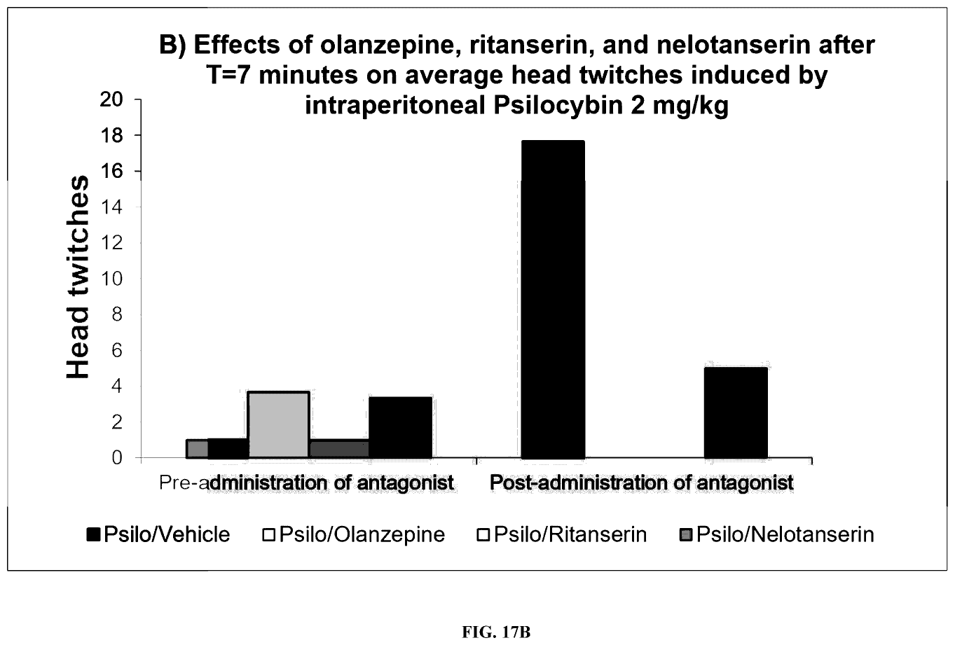

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (DMSO:HPCD [10:90]) or ritanserin (1 mg/kg) or nelotanserin (1 mg/kg) or olanzapine (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 17, administration of olanzapine and ritanserin completely suppressed the head-twitch response, and administration of nelotanserin partially suppressed the head-twitch response. FIG. 17A shows a graph, FIG. 17B shows a bar chart showing head twitches occurring before and after control or antagonist administration. Test condition groups of Example 11 are summarized in Table 12.

TABLE 12
Synopsis of mouse twitch test pilot schedule

| | 60 min pretest | | | T = 7 min | T = 0-40 min |
|---|---|---|---|---|---|
| | Place in | T = −3 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | Psilocybin 2 mg/kg | Vehicle 1 | Yes |
| 3 | Yes | Yes | Psilocybin 2 mg/kg | Ritanserin 1 mg/kg | Yes |
| 3 | Yes | Yes | Psilocybin 2 mg/kg | Nelotanserin 1 mg/kg | Yes |
| 3 | Yes | Yes | Psilocybin 2 mg/kg | Olanzapine 1 mg/kg | Yes |

### Example 12: Effect of Intravenous Administration of Quetiapine on LSD-Induced Head Twitches in Mice

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Quetiapine was dissolved in Vehicle 1 (saline) at concentrations of 0.2 mg free base equivalents/ml to give a dose of 1 mg/kg when administered i.v. in a 5 mL/kg dosing volume.

### Procedure

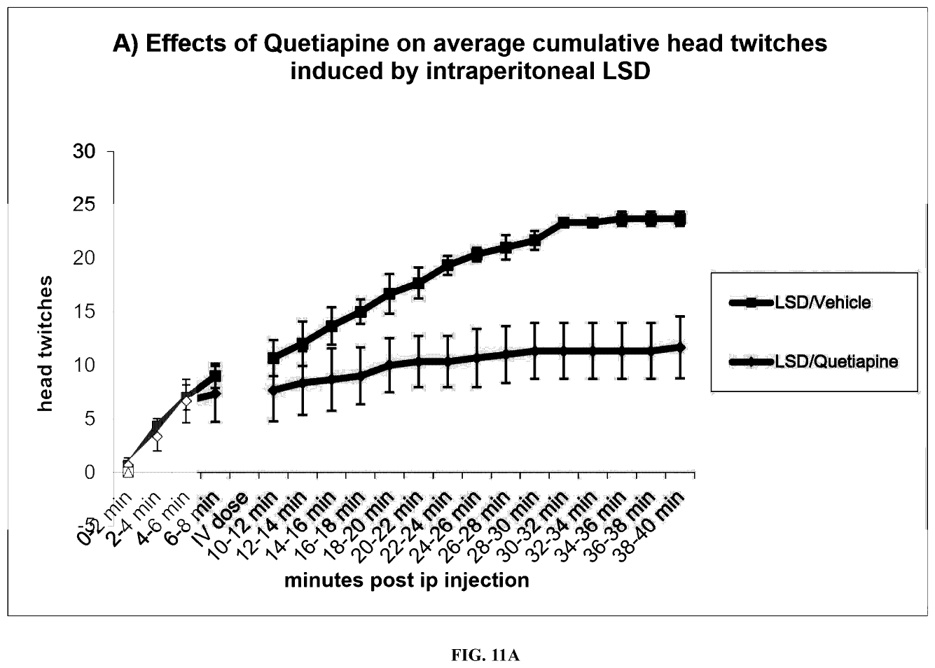

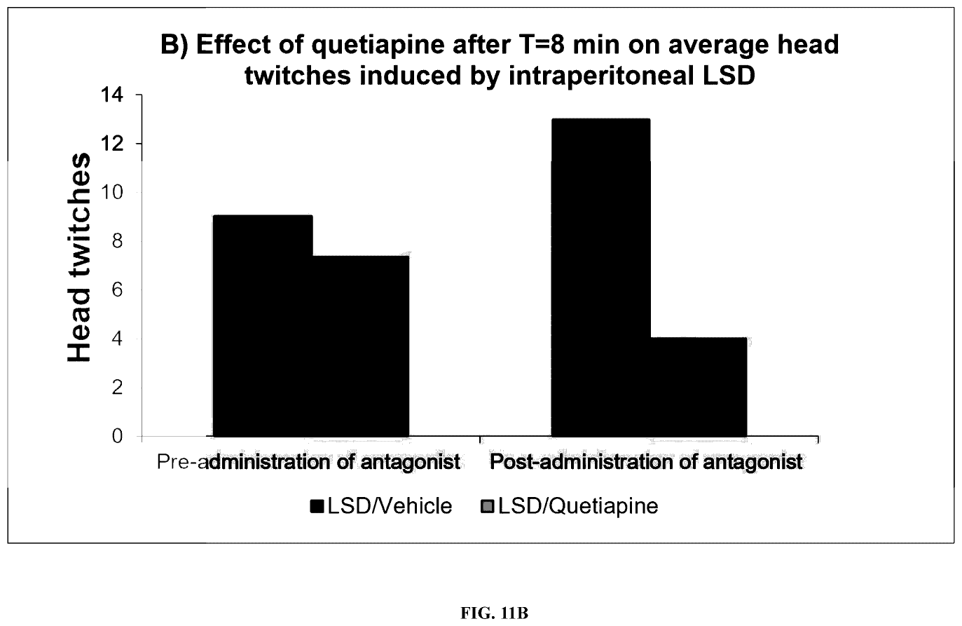

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 1 (saline) or quetiapine (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 11, administration of quetiapine partially suppressed the head-twitch response. FIG. 11A shows a graph, FIG. 11B shows a bar chart showing head twitches occurring before and after control or antagonist administration. Test condition groups of Example 12 are summarized in Table 13.

TABLE 13
Synopsis of mouse twitch test pilot schedule

| | 60 min pre-test | | | T = 8 min | T = 0-40 min |
|---|---|---|---|---|---|
| | Place in | T = −2 min | T = 0 pretest | Treatment i.v. | Assess number of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | LSD 0.32 mg/kg | Vehicle 1 | Yes |
| 3 | Yes | Yes | LSD 0.32 mg/kg | Quetiapine1 mg/kg | Yes |

### Example 13A: Effect of Intravenous Administration of Quetiapine on Psilocybin Induced Head Twitches in Mice

### Formulation

Psilocybin (free base) was formulated in saline at a concentration of 0.4 mg/mL to give a dose of 2 mg/kg when administered i.p. in a 5 mL/kg dosing volume. Quetiapine was dissolved in Vehicle 1 (saline) at concentrations of 0.2 mg free base equivalents/ml to give a dose of 1 mg/kg when administered i.v. in a 5 mL/kg dosing volume.

### Procedure

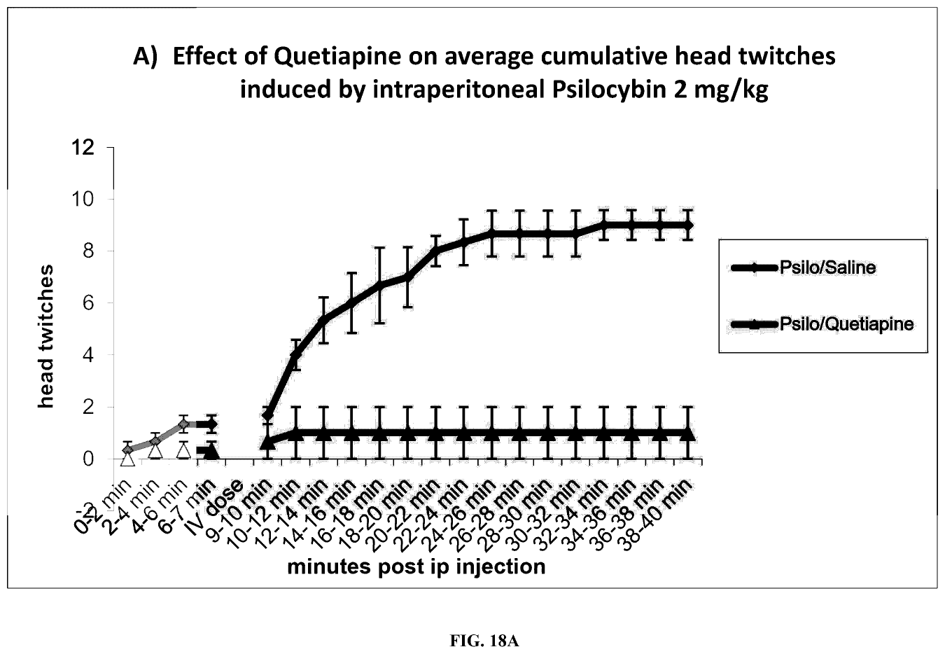

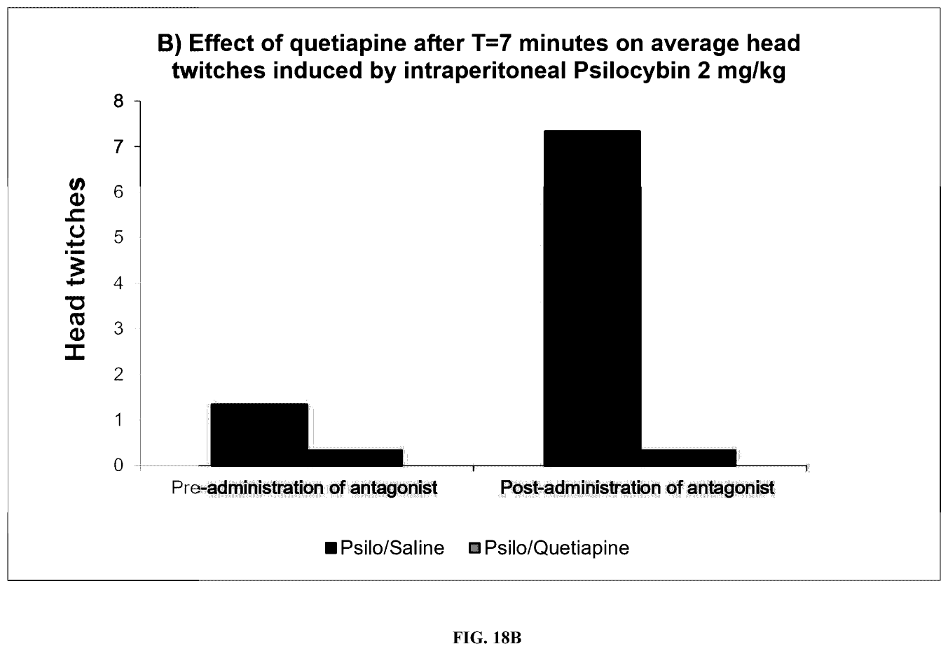

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (saline) or quetiapine (1 mg/kg) in vehicle 1 in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 18, administration of quetiapine completely suppressed the head-twitch response. FIG. 18A shows a graph, FIG. 18B shows a bar chart showing head twitches occurring before and after control or antagonist administration. Test condition groups of Example 13A are summarized in Table 14.

TABLE 14
Synopsis of mouse twitch test pilot schedule

| | 60 min pre-test | | | T = 7 min | T = 0-40 min |
|---|---|---|---|---|---|
| | Place in | T = −3 min | T = 0 pretest | Treatment i.v. | Assess no of |
| Group | test cage to | Place in | Treatment i.p. | (5 mL/kg) and | head shakes/40 |
| (n) | habituate | heated cage | (5 mL/kg saline) | replace in test cage | min per mouse |
| 3 | Yes | Yes | Psilocybin 2 mg/kg | Vehicle 1 (saline) | Yes |
| 3 | yes | yes | Psilocybin 2 mg/kg | Quetiapine 1 mg/kg | Yes |

### Example 13B: Effect of Intravenous Administration of Ketanserin on Psilocybin Induced Head Twitches in Mice

### Formulation

Psilocybin (free base) was formulated in saline at a concentration of 0.4 mg/mL to give a dose of 2 mg/kg when administered i.p. in a 5 mL/kg dosing volume.

Ketanserin Tartrate (74.7% free base content) was dissolved in Vehicle 2 (DMSO: Cremophor EL: Hydroxypropyl-o-cyclodextrin (20% in water) [10:10:80]) at concentrations of 0.4 mg/ml to give a dose of 2 mg/kg when administered i.v. in a 5 mL/kg dosing volume.

### Procedure

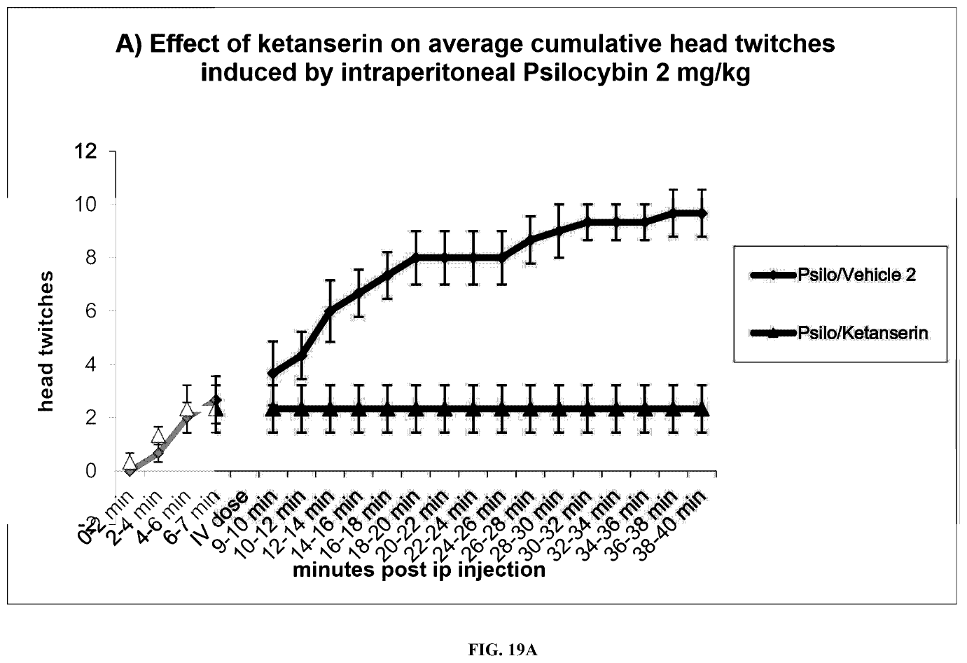

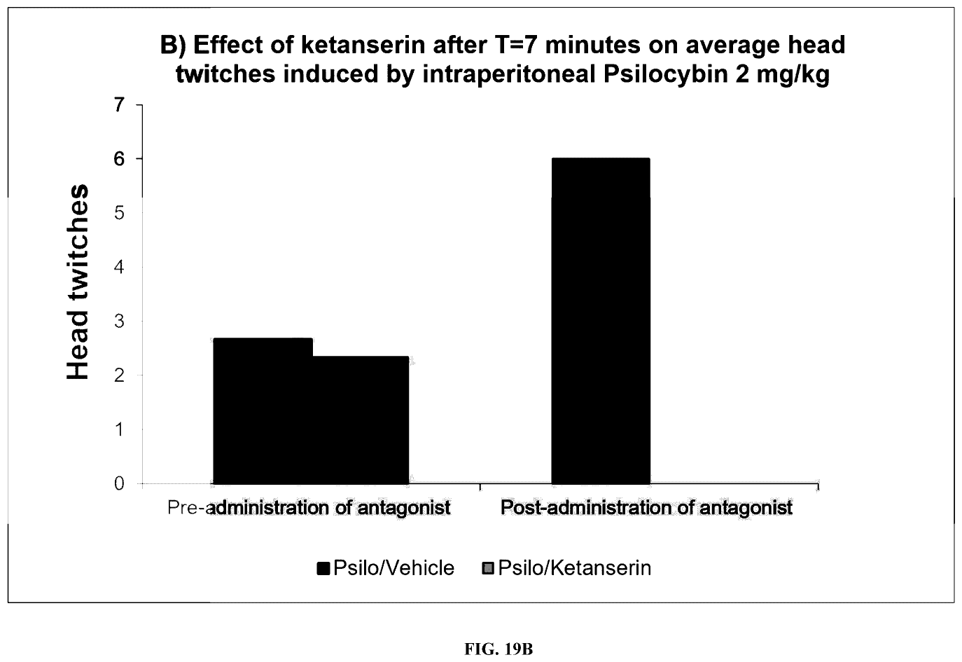

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either Vehicle 2 (DMSO:cremophor:HPCD [10:10:80]) or ketanserin (2 mg/kg) in Vehicle 2 in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 19, administration of ketanserin completely suppressed the head-twitch response. FIG. 19A shows a graph, FIG. 19B shows a bar chart showing head twitches occurring before and after control or antagonist administration. Test condition groups of Example 13B are summarized in Table 15.

TABLE 15
Synopsis of mouse twitch test pilot schedule

| | 60 min pretest | | | T = 7 min | T = 0-40 min |
|---|---|---|---|---|---|
| | Place in | T = −3 min | T = 0 pretest | Treatment i.v. | Assess no of |
| Group | test cage to | Place in | Treatment i.p. | (5 mL/kg) and | head shakes/40 |
| (n) | habituate | heated cage | (5 mL/kg saline) | replace in test cage | min per mouse |
| 3 | yes | yes | Psilocybin 2 mg/kg | Vehicle 2 | Yes |
| | | | | (DMSO:Cremophor:HPCD) | |
| 3 | yes | yes | Psilocybin 2 mg/kg | Ketanserin 2 mg/kg | Yes |

### Example 14: Effect of Intravenous Administration of AC-279 on LSD- and Psilocybin-Induced Head Twitches in Mice

### Formulation Check

AC-279 was assessed in up to 4 different formulations to identify a vehicle suitable for i.v. administration in which it forms a solution at a concentrations of 0.2 mg free base equivalents/ml to give a dose of 1 mg/kg when administered i.v. in a 5 mL/kg dosing volume.

### Formulation

Lysergic acid diethylamine tartrate (LSD tartrate, 67.2% free base content) was formulated in Vehicle 1 (saline) at a concentration of 0.063 mg free base/mL to give doses of 0.32 mg/kg when administered i.p. in a 5 mL/kg dosing volume.

Psilocybin (free base) was formulated in Vehicle (saline) at a concentration of 0.4 mg/mL to give a dose of 2 mg/kg when administered i.p. in a 5 mL/kg dosing volume.

AC-279 was dissolved in Vehicle 1 (10% DMSO:90% HPCD) at concentrations of 0.2 mg free base equivalents/ml to give a dose of 1 mg/kg when administered i.v. in a 5 mL/kg dosing volume.

### Procedure 1: LSD

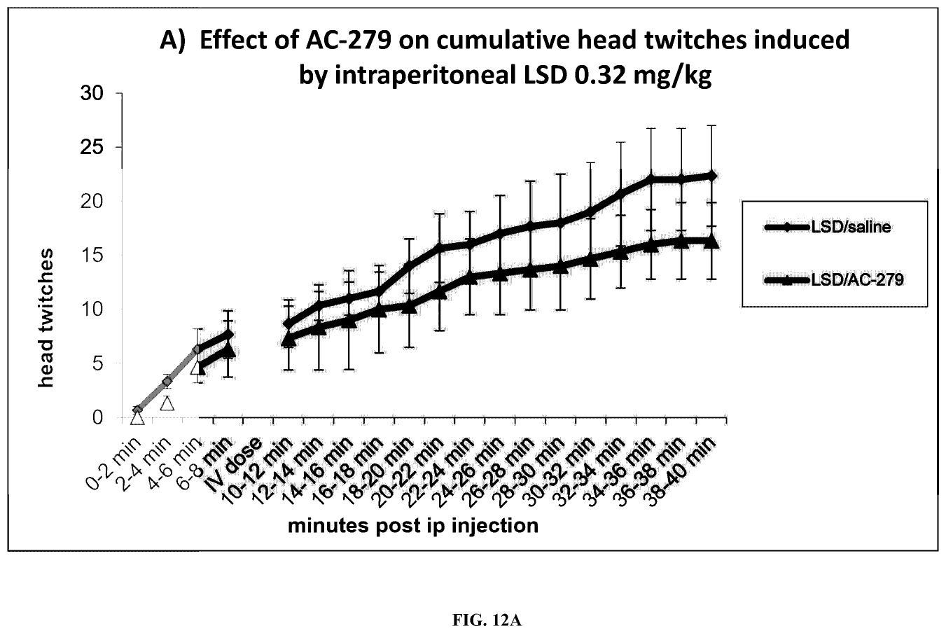

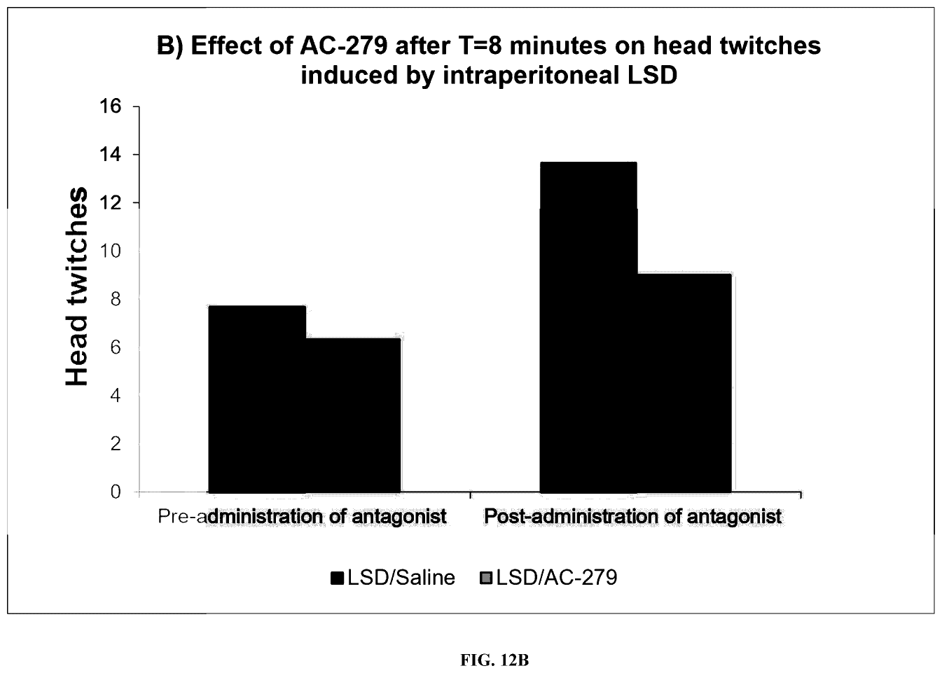

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle (saline) or AC-279 (1 mg/kg) in 5 mL/kg dosing volumes. Mice were then replaced into the observation cages and head twitch behavior was monitored until 40 min after agonist dosing. Cumulative head-twitch behavior was measured every 2 minutes until 40 minutes after agonist dosing. As shown in FIG. 12, administration of AC-279 did not suppress the head-twitch response. FIG. 12A shows a graph, FIG. 12B shows a bar chart showing head twitches occurring before and after control or antagonist administration. Procedure 2: Psilocybin

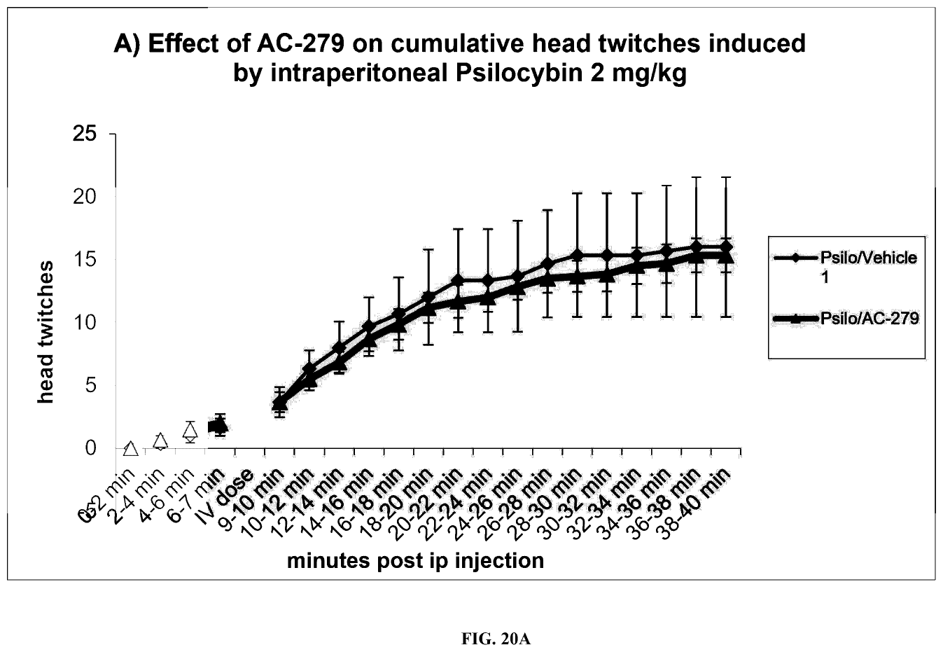

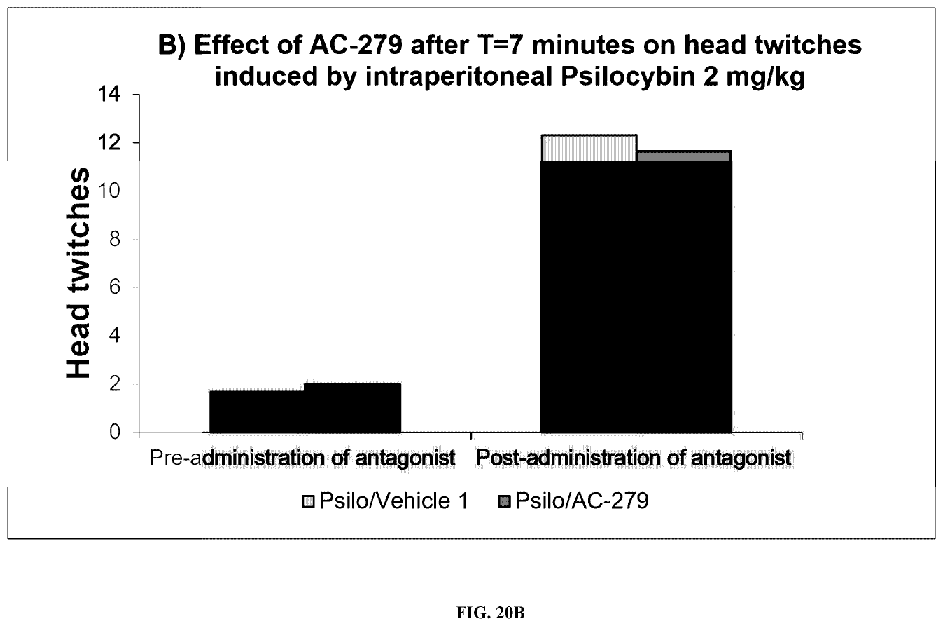

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with vehicle 1 (10% DMSO:90% HPCD) or AC-279 (1 mg/kg) in 5 mL/kg dosing volumes. Mice were replaced into the observation cages and head twitch behavior was monitored until 40 min after agonist dosing. Cumulative head-twitch behavior was measured every 2 minutes until 40 minutes after agonist dosing. As shown in FIG. 20, administration of AC-279 did not suppress the head-twitch response. FIG. 20A shows a graph, FIG. 20B shows a bar chart showing head twitches occurring before and after control or antagonist administration. Procedures 1 and 2 test condition groups of Example 14 are summarized in Table 16.

TABLE 16
Synopsis of mouse twitch test study schedule

| | 60 min pretest | | | T = 8 min | T = 0-40 min |
|---|---|---|---|---|---|
| | Place in | T = −2 min | T = 0 pretest | Treatment i.v. | Assess no of |
| Group | test cage to | Place in | Treatment i.p. | (5 mL/kg) and | head shakes/40 |
| (n) | habituate | heated cage | (5 mL/kg saline) | replace in test cage | min per mouse |
| 3 | Yes | Yes | LSD 0.32 mg/kg | Vehicle (saline) | Yes |
| 3 | Yes | Yes | LSD 0.32 mg/kg | AC-279 1 mg/kg | Yes |
| | 60 min pretest | T = −3 min | T = 0 pretest | T = 7 min | T = 0-40 min |
| 3 | yes | yes | Psilocybin 2 mg/kg | Vehicle 1 | Yes |
| | | | | (DMSO:HPCD) | |
| 3 | yes | yes | Psilocybin 2 mg/kg | AC-279 1 mg/kg | Yes |

### Example 15: Effect of Intravenous Administration of Eplivanserin on LSD-Induced Head Twitches in Mice

### Formulation

Lysergic acid diethylamine tartrate (LSD tartrate, 67.2% free base content) was formulated in saline at a concentration of 0.063 mg free base/mL to give doses of 0.32 mg/kg when administered i.p. in a 5 mL/kg dosing volume. Eplivanserin was dissolved in Vehicle 1 (DMSO:HPCD [10:90]) at concentrations of 0.2 mg free base equivalents/ml to give a dose of 1 mg/kg when administered i.v. in a 5 mL/kg dosing volume.

### Procedure

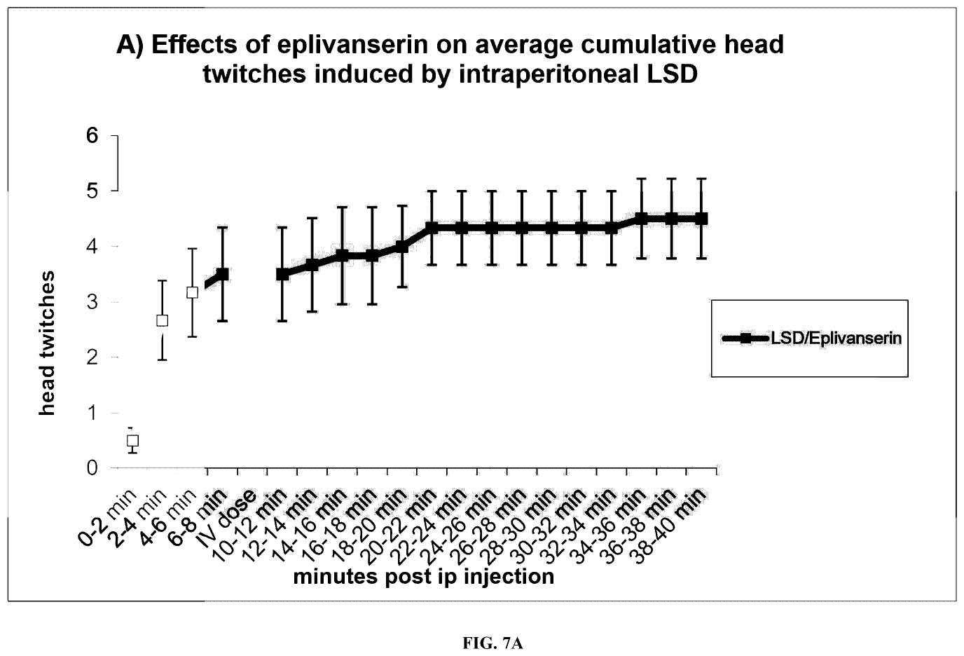

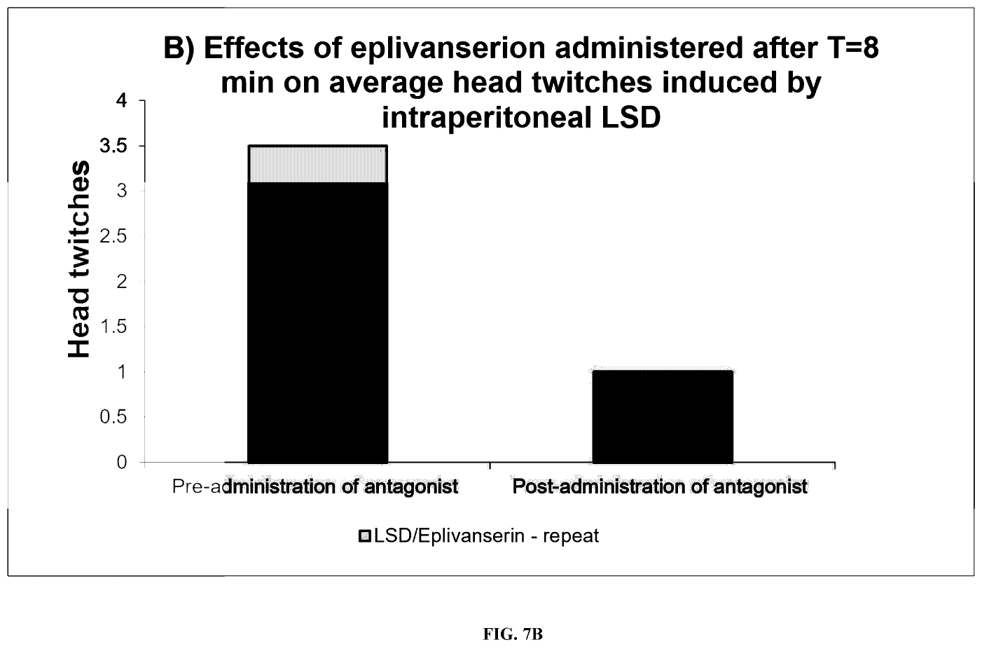

At T=−60 min, C57BL/6J mice were individually housed into transparent observation cages with bedding removed and left to habituate. At T=−2 min, mice were placed into heated cages at 40° C. At T=0 h, all 6 mice were dosed intraperitoneally with LSD (0.32 mg/kg). Following dosing, mice will be replaced into the heated cages and head-twitch responses continuously scored for 8 min. After 8 min, mice were intravenously dosed via the lateral tail vein with eplivanserin (1 mg/kg) in 5 mL/kg dosing volumes. Mice were then replaced into the observation cages and head twitch behavior was monitored until 40 min after agonist dosing. Cumulative head-twitch behavior was measured every 2 minutes until 40 minutes after agonist dosing. As shown in FIG. 7, administration of eplivanserin (in n=6 mice) partially suppressed the head-twitch response. FIG. 7A shows a graph, FIG. 7B shows a bar chart showing head twitches occurring before and after control or antagonist administration. Test condition groups of Example 15 are summarized in Table 17.

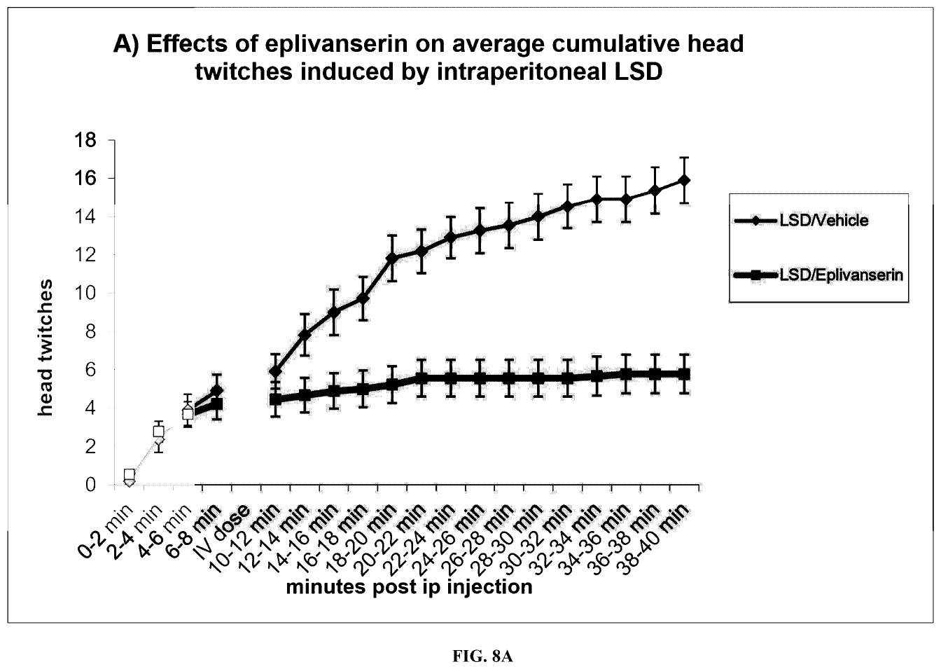

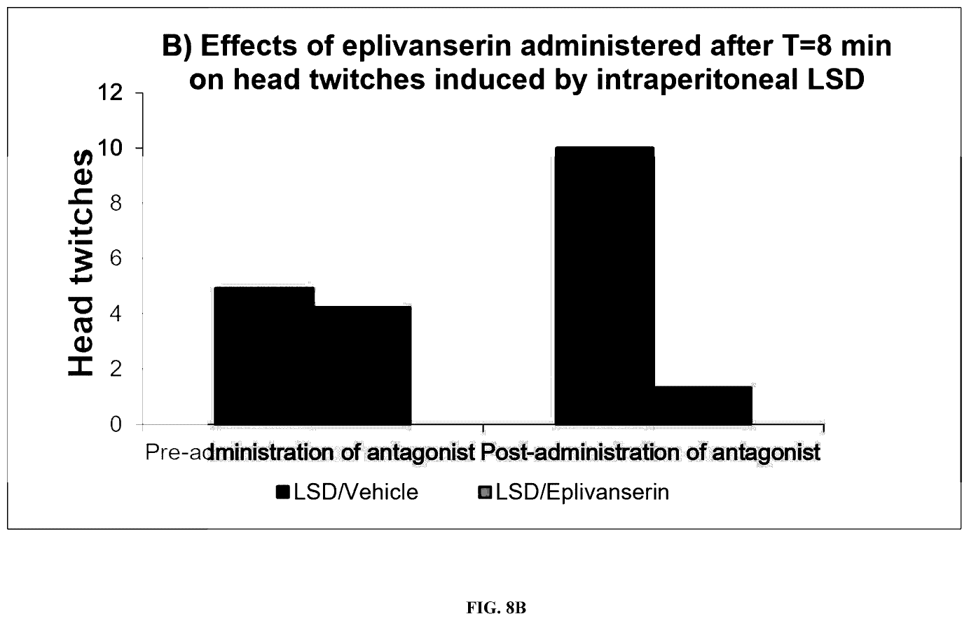

The results from Example 6 and Example 15 were pooled and graphed to illustrate the average effects of eplivanserin on cumulative head twitches induced by intraperitoneal LSD administration from the two protocols. Mice were dosed intraperitoneally with LSD (0.32 mg/kg) and head-twitch responses were continuously scored for 8 minutes. After 8 minutes, mice were intravenously dosed with either vehicle (DMSO:HPCD [10:90]) or eplivanserin (1 mg/kg). Cumulative head-twitch behavior was measured every 2 minutes until 40 minutes after agonist dosing. The plots show the average head twitches in mice dosed with LSD and eplivanserin in Example 6 (n=3) and Example 15 (n=6), compared to the average head twitches in mice dosed with LSD and the vehicle (DMSO:HPCD [10:90]) in Example 6 and across all other studies with the same vehicle. As shown in FIG. 8, administration of eplivanserin partially suppressed the head-twitch response. FIG. 8A shows a graph, FIG. 8B shows a bar chart showing head twitches occurring before and after control or antagonist administration.

TABLE 17
Synopsis of mouse twitch test pilot schedule

| | 60 min pretest | | | T = 8 min | T = 0-40 min |
|---|---|---|---|---|---|
| | Place in | T = −2 min | T = 0 pretest | Treatment i.v. | Assess no of |
| Group | test cage to | Place in | Treatment i.p. | (5 mL/kg) and | head shakes 40 |
| (n) | habituate | heated cage | (5 mL/kg saline) | replace in test cage | min per mouse |
| 6 | yes | yes | LSD 0.32 mg/kg | Eplivanserin 1 mg/kg | Yes |

### Example 16: Effect of Intravenous Administration of Flibanserin on LSD- and Psilocybin-Induced Head Twitches in Mice

### Formulation

Lysergic acid diethylamine tartrate (LSD tartrate, 67.2% free base content) was formulated in Vehicle 1 (saline) at a concentration of 0.063 mg free base/mL to give doses of 0.32 mg/kg when administered i.p. in a 5 mL/kg dosing volume.

Psilocybin (free base) was formulated in Vehicle 1 (saline) at a concentration of 0.4 mg/mL to give a dose of 2 mg/kg when administered i.p. in a 5 mL/kg dosing volume.

Flibanserin was dissolved in Vehicle 2 (DMSO:HPCD (20% w/v in water) [10:90]) at concentrations of 0.2 and 0.8 mg free base equivalents/ml to give doses of 1 and 4 mg/kg when administered i.v. in 5 mL/kg dosing volumes.

### Procedure 1: LSD

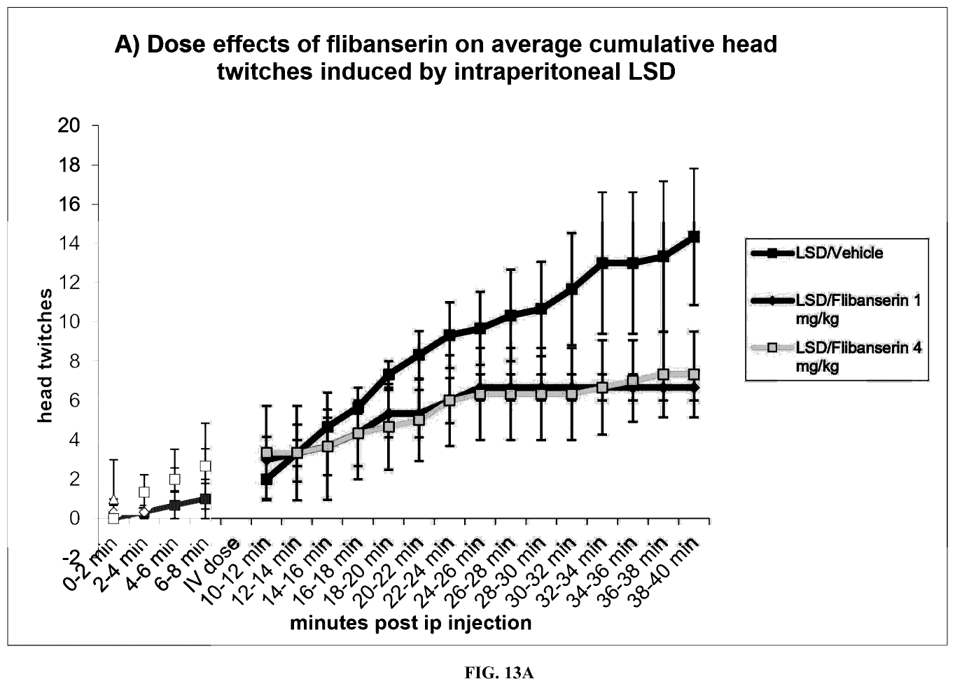

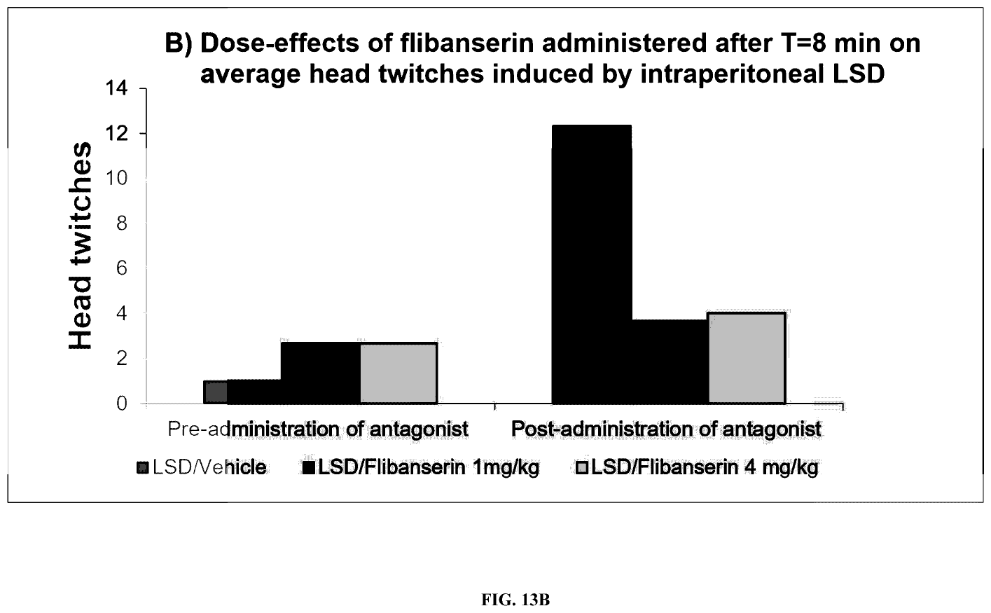

At T=−60 min, C57BL/6J mice were individually housed into transparent observation cages with bedding removed and left to habituate. At T=−2 min, mice were placed into heated cages at 40° C. At T=0 h, groups of 3 mice were dosed intraperitoneally with LSD (0.32 mg/kg). Following dosing, mice were replaced into the heated cages and head-twitch responses continuously scored for 8 min. After 8 min, mice were intravenously dosed via the lateral tail vein with either vehicle 2 (DMSO:HPCD) or Flibanserin (1 mg/kg or 4 mg/kg) in 5 mL/kg dosing volumes. Mice were then replaced into the observation cages and head twitch behavior was monitored until 40 min after agonist dosing. Cumulative head-twitch behavior was measured every 2 minutes until 40 minutes after agonist dosing. As shown in FIG. 13, administration of flibanserin (1 mg/kg or 4 mg/kg) partially suppressed the head-twitch response. FIG. 13A shows a graph, FIG. 13B shows a bar chart showing head twitches occurring before and after control or antagonist administration.

### Procedure 2: Psilocybin

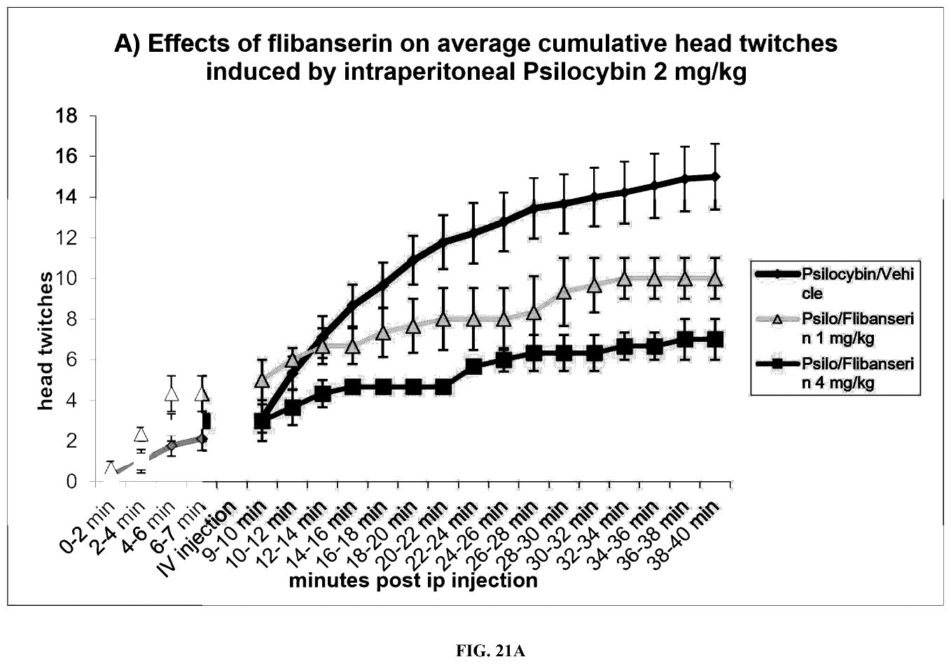

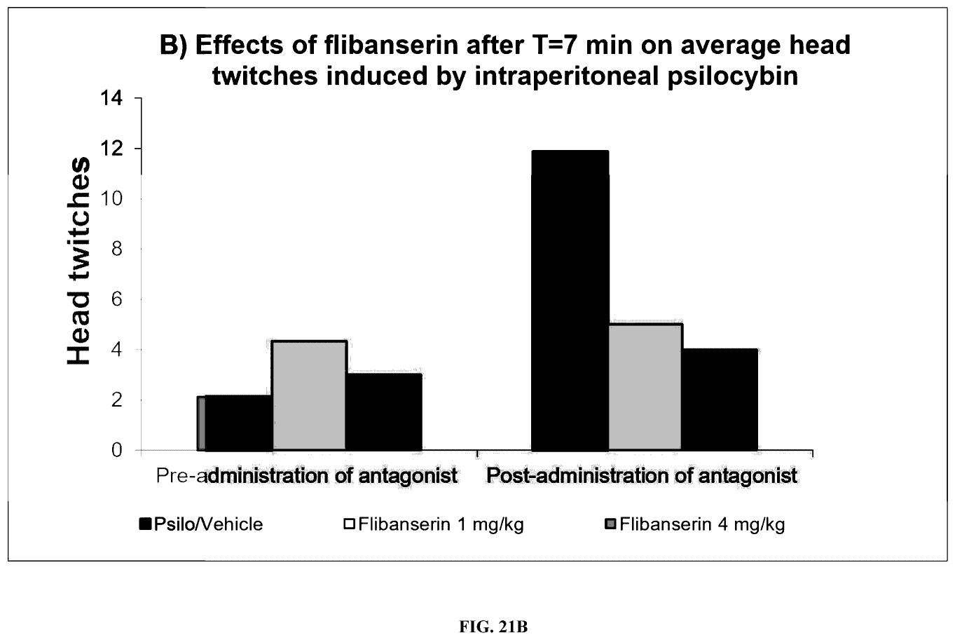

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with Flibanserin (1 or 4 mg/kg) in 5 mL/kg dosing volumes. Mice were then replaced into the observation cages and head twitch behavior was monitored until 40 min after agonist dosing. Since this combination of Psilocybin and vehicle had already been tested multiple times, a separate control group was not run concurrently to minimize the number of animals required for the study. Instead, the control groups from the other psilocybin and studies with the same vehicle 2 and dosing schedule were all averaged together. Cumulative head-twitch behavior was measured every 2 minutes until 40 minutes after agonist dosing. The average cumulative head twitches in mice dosed with psilocybin and then the same vehicle (DMSO:HPCD [10:90]) from the other studies is plotted for reference. As shown in FIG. 21, administration of flibanserin (1 mg/kg or 4 mg/kg) partially suppressed the head-twitch response. FIG. 21A shows a graph, FIG. 21B shows a bar chart showing head twitches occurring before and after control or antagonist administration. Procedures 1 and 2 test condition groups of Example 16 are summarized in Table 18.

TABLE 18
Synopsis of mouse twitch test study schedule

| | 60 min pre-test | | | T = 8 min | T = 0-40 min |
|---|---|---|---|---|---|
| | Place in | T = −2 min | T = 0 pretest | Treatment i.v. | Assess no of |
| Group | test cage to | Place in | Treatment i.p. | (5 mL/kg) and | head shakes/40 |
| (n) | habituate | heated cage | (5 mL/kg saline) | replace in test cage | min per mouse |
| 3 | Yes | Yes | LSD 0.32 mg/kg | Vehicle 2 | Yes |
| | | | | (DMSO:HPCD) | |
| 3 | yes | yes | LSD 0.32 mg/kg | Flibanserin 1 mg/kg | Yes |
| 3 | yes | yes | LSD 0.32 mg/kg | Flibanserin 4 mg/kg | Yes |
| | 60 min pre-test | T = −3 min | T = 0 pretest | T = 7 min | T = 0-40 min |
| 9 | Yes | yes | Psilocybin 2 mg/kg | Vehicle 2 | Yes |
| | | | | (DMSO:HPCD) | |
| 3 | yes | yes | Psilocybin 2 mg/kg | Flibanserin 1 mg/kg | Yes |
| 3 | yes | yes | Psilocybin 2 mg/kg | Flibanserin 4 mg/kg | Yes |

### Example 17: Effect of Intravenous Administration of Nelotanserin on Psilocybin Induced Head Twitches in Mice

### Formulation

Psilocybin (free base) was formulated in saline at a concentration of 0.4 mg/mL to give a dose of 2 mg/kg when administered i.p. in a 5 mL/kg dosing volume. Nelotanserin (free base) was dissolved in Vehicle 1 (DMSO:Kolliphor:HPCD (20% in water) [10:10:80]) at concentrations of 0.8 mg free base equivalents/ml to give a dose of 4 mg/kg when administered i.v. in a 5 mL/kg dosing volume.

### Procedure

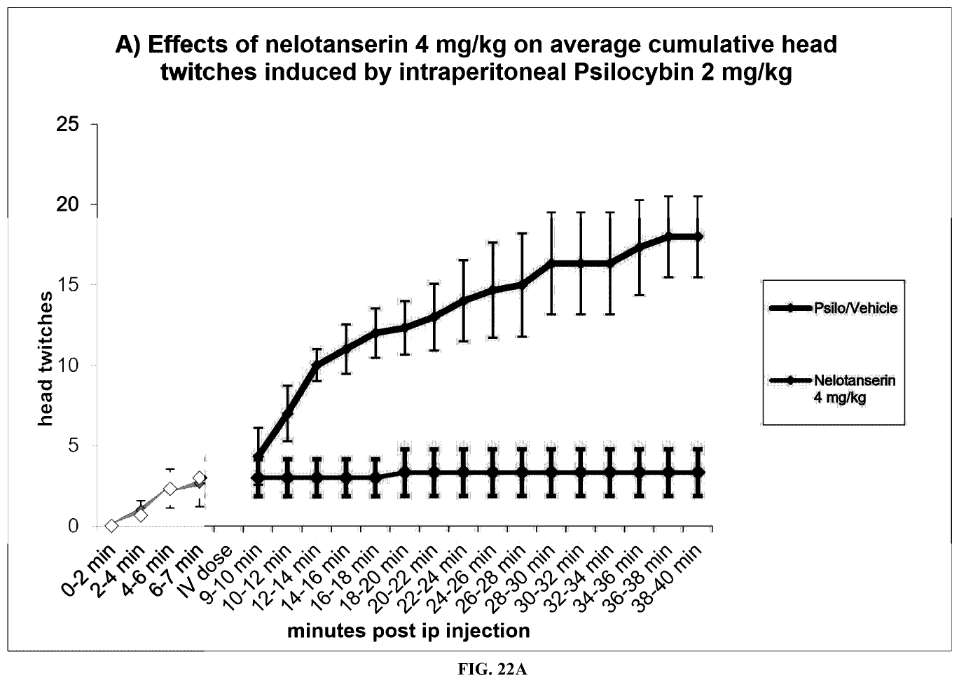

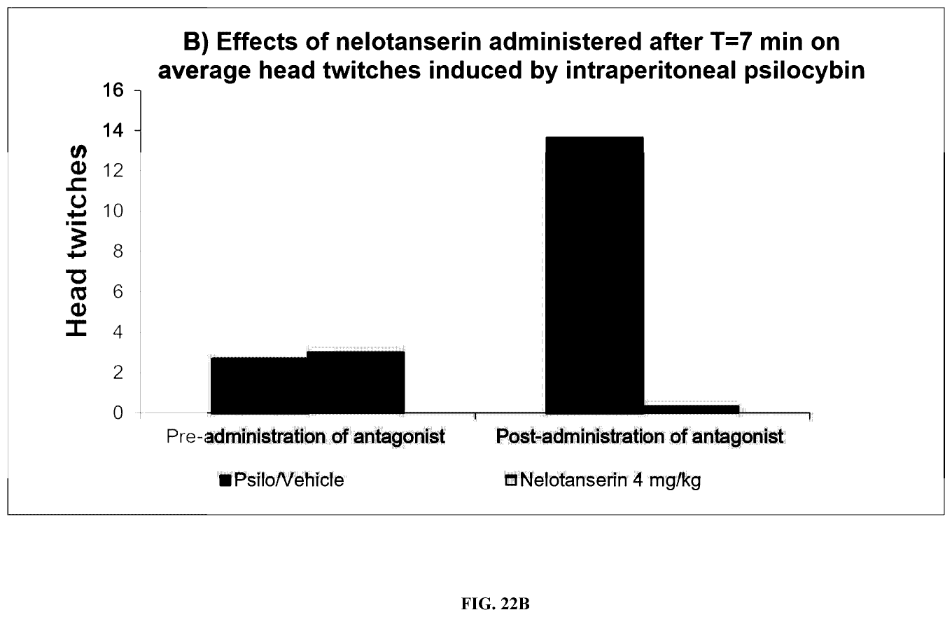

At T=−60 min, C57BL/6J mice were individually housed into transparent observation cages with bedding removed and left to habituate. At T=−3 min, mice were placed into heated cages at 40° C. At T=0 h, groups of 3 mice were dosed intraperitoneally with psilocybin (2 mg/kg). Following dosing, mice were replaced into the heated cages and head-twitch responses continuously scored for 7 min. After 7 min, mice were intravenously dosed via the lateral tail vein with either vehicle 1 (DMSO:Kolliphor:HPCD) or nelotanserin (4 mg/kg) in 5 mL/kg dosing volumes. Mice were then replaced into the observation cages and head twitch behavior was monitored until 40 min after agonist dosing. Cumulative head-twitch behavior was measured every 2 minutes until 40 minutes after agonist dosing. As shown in FIG. 22, administration of higher dose nelotanserin (4 mg/kg) completely suppressed the head-twitch response. FIG. 22A shows a graph, FIG. 22B shows a bar chart showing head twitches occurring before and after control or antagonist administration. Test condition groups of Example 17 are summarized in Table 19.

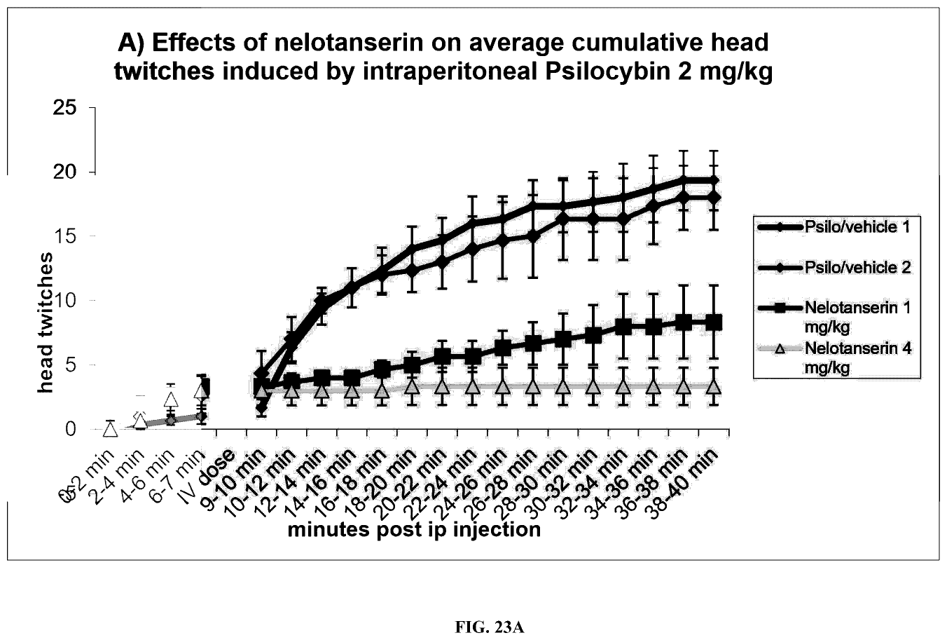

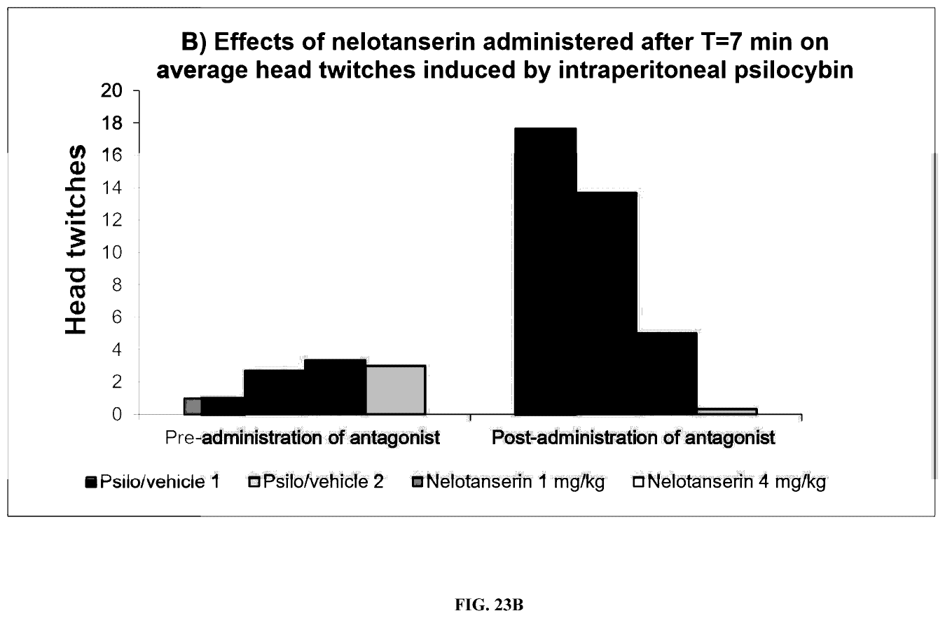

The results from Example 11 and Example 17 were analyzed in combination and graphed to illustrate the effects of nelotanserin dosage on cumulative head twitches induced by intraperitoneal psilocybin administration from the two protocols. Mice were dosed intraperitoneally with psilocybin (2 mg/kg) and head-twitch responses were continuously scored for 7 minutes. After 7 minutes, mice were intravenously dosed with either vehicle 1 (DMSO:HPCD [10:90]) or nelotanserin (ling/kg) in Example 11, or with either vehicle 2 (DMSO:Kolliphor:HPCD (20% in water)=10:10:80) or nelotanserin (4 mg/kg) in Example 17. Cumulative head-twitch behavior was measured every 2 minutes until 40 minutes after agonist dosing. As shown in FIG. 23, administration of nelotanserin at 1 mg/kg partially suppressed the head-twitch response, and administration of nelotanserin at 4 mg/kg completed suppressed the response. This indicated a dose-responsive effect of nelotanserin in suppression of psilocybin-induced head twitches. FIG. 23A shows a graph, FIG. 23B shows a bar chart showing head twitches occurring before and after control or antagonist administration.

TABLE 19
Synopsis of mouse twitch test pilot schedule

| | 60 min pre-test | | | T = 7 min | T = 0-40 min |
|---|---|---|---|---|---|
| | Place in | T = −3 min | T = 0 pretest | Treatment i.v. | Assess no of |
| Group | test cage to | Place in | Treatment i.p. | (5 mL/kg) and | head shakes 40 |
| (n) | habituate | heated cage | (5 mL/kg saline) | replace in test cage | min per mouse |
| 3 | Yes | Yes | Psilocybin 2 mg/kg | Vehicle 1 | Yes |
| | | | | (DMSO:Kolliphor:HPCD) | |
| 3 | yes | yes | Psilocybin 2 mg/kg | Nelotanserin 4 mg/kg | Yes |

### Example 18. Results

Table 20 is a list of all combinations tested of various 5HT-2A receptor antagonists with LSD-treated animals and a summary description of the results from the examples (Examples 1-17) herein above.

TABLE 20
LSD Combinations tested

| | 5HT2A | |
|---|---|---|
| Psychedelic | antagonist | Result |
| LSD | Volinanserin | Full suppression of head twitch at |
| | | all 3 timepoints tested |
| LSD | Eplivanserin | Partial suppression of head twitch |
| LSD | Pimavanserin | Partial suppression of head twitch |
| LSD | Pruvanserin | Full suppression of head twitch |
| LSD | Nelotanserin | Partial suppression of head twitch |
| LSD | Ritanserin | Full suppression of head twitch |
| LSD | Risperidone | Full suppression of head twitch |
| LSD | Olanzapine | Full suppression of head twitch |
| LSD | Quetiapine | Partial suppression of head twitch |
| LSD | AC-279 | very minimal suppression of head twitch |
| | (N-Desmethyl | |
| | Pimavanserin) | |
| LSD | Flibanserin | Partial suppression of head twitch |

Table 21 is a list of all combinations tested of various 5HT-2A receptor antagonists with psilocybin-treated animals and a summary description of the results from the examples (Examples 1-17) herein above.

TABLE 21
Psilocybin Combinations tested

| | 5HT2A | |
|---|---|---|
| Psychedelic | antagonist | Result |
| Psilocybin | Volinanserin | Full suppression of head twitch |
| Psilocybin | Eplivanserin | Full suppression of head twitch |
| Psilocybin | Pimavanserin | Full suppression of head twitch |
| Psilocybin | Pruvanserin | Full suppression of head twitch |
| Psilocybin | Nelotanserin | Partial suppression of head twitch, |
| | | full suppression at high dose |
| Psilocybin | Ritanserin | Full suppression of head twitch |
| Psilocybin | Risperidone | Full suppression of head twitch |
| Psilocybin | Olanzapine | Full suppression of head twitch |
| Psilocybin | Quetiapine | Full suppression of head twitch |
| Psilocybin | AC-279 | No suppression of head twitch |
| | (N-Desmethyl | |
| | Pimavanserin) | |
| Psilocybin | Flibanserin | Partial suppression of head twitch |

Described in Table 22 are ranges of dosing of various 5HT-2A receptor antagonists in order to achieve a rapid ending of the hallucinogenic effects of a psychedelic trip.

TABLE 22
Dosing for psychedelic trip ending

| | Dose administered as a monotherapy in an oral or I.V. or intranasal |
|---|---|
| Antagonist | dosage form to end or reduce the intensity of a psychedelic trip |
| Volinanserin | 1 mg to about 60 mg, or about 5 mg to about 20 mg |
| Pimavanserin | 1 mg to about 60 mg, or about 17 mg to about 34 mg |
| Ketanserin | about 10 mg to about 80 mg, about 30 mg to about 50 mg, |
| | or about 40 mg |
| Pruvanserin | 1 mg to about 40 mg, or about 3 mg to about 10 mg |
| Nelotanserin | 1 mg to about 80 mg, or about 40 mg to about 80 mg |
| Ritanserin | 1 mg to about 40 mg, or about 2.5 mg to about 10 mg |
| Eplivanserin | 1 mg to about 40 mg, or about 5 mg to about 10 mg |
| Flibanserin | 10 mg to about 200 mg, or about 80 mg to about 120 mg, |
| | or about 100 mg |
| Risperidone | 0.5 mg to about 20 mg or about.5 mg, or about 1 mg, |
| | or about 2 mg, or about 3 mg or about 4 mg or about |
| | 5 mg or about 7.5 mg or about 10 mg or about 16 mg |
| Olanzapine | about 2.5 mg to about 30 mg, or about 5 mg or about 7.5 mg or |
| | about 10 mg, or about 15 mg or about 20 mg or about 25 mg |
| Quetiapine | about 25 mg to about 800 mg, or about 50 mg to about 100 mg, or |
| | about 150 mg or about 200 mg or about 250 mg or about 300 mg |
| extended-release of | 50 mg to about 450 mg, or about 150 mg or about 210 mg, or |
| olanzapine (e.g., | about 300 mg or about 405 mg |
| ZYPREXA RELPREVV) | |
| extended-release of | 50 mg to about 300 mg, or about 50 mg or about 100 mg or about |
| quetiapine | 200 mg, or about 300 mg |
| extended-release of | 12.5 mg, or about 25 mg, or about 37.5 mg, or about 50 mg |
| risperidone (e.g., | |
| RISPERDAL CONSTA) | |

### Discussion

As shown in the examples above and in Table 20, the selective 5HT2A antagonists volinanserin and pruvanserin, and the selective 5HT2A/5HT2C antagonist ritanserin resulted in full suppression of LSD-induced head twitch compared to vehicle treated mice. The selective 5HT2A antagonists eplivanserin, pimavanserin, nelotanserin, and flibanserin resulted in partial suppression of LSD's psychedelic effects when administered after LSD, indicating that these selective 5HT2A antagonists reduced LSD-induced psychedelic effects. The active metabolite of pimavanserin, i.e., AC-279 (N-desmethyl Pimavanserin) showed only very minimal effects on LSD-induced psychedelic effects. Finally, the atypical antipsychotics risperidone and olanzapine resulted in full ending of LSD induced head twitch while quetiapine resulted in partial reduction in the effects. As shown in Table 21, selective 5HT2A antagonists volinanserin, eplivanserin, pimavanserin, and pruvanserin, and selective 5HT2A/5HT2C antagonist ritanserin resulted in full ending of psilocybin-induced head twitch compared to in vehicle-treated mice. Selective 5HT2A antagonists nelotanserin and flibanserin resulted in partial suppression of psilocybin's psychedelic effects when administered after psilocybin, indicating that nelotanserin and flibanserin reduced psilocybin-induced psychedelic effects. The active metabolite of pimavanserin, i.e., AC-279 (N-desmethyl Pimavanserin) did not have significant effects in reducing psilocybin-induced psychedelic effects. The atypical antipsychotics risperidone, olanzapine, and quetiapine resulted in full suppression of psilocybin induced head twitch. These results showed that the 5HT2A antagonists in Table 20 and Table 21, when administered after psychedelics (such as LSD and psilocybin) could end or reduce the intensity of hallucinations induced by psychedelics (such as LSD and psilocybin).

The antagonists in Table 20 and Table 21 could be administered after LSD, Psilocybin, or other psychedelics to tailor the duration or intensity of psychedelic trips. Some of the benefits of administering the antagonists in Table 20 and Table 21 include tailoring psychedelic trips to durations that are suitable for psychedelic-assisted psychotherapy, ending bad trips, reducing the intensity of bad trips, treating psychosis induced by psychedelics, and treating overdose of psychedelics. The optimal duration of psychedelic assisted psychotherapy for a patient could be determined by a person of skilled in the art (e.g., a physician) to control the duration of the psychedelic effects to the optimal duration for each individual patient. This allows healthcare providers to tailor psychedelic trips for individual patients to minimize bad trips. By administering the antagonists in Table 20 and Table 21, each patient may receive a different and unique duration of therapy and a unique intensity of therapy that has been determined to be optimal for that patient. This allows personalized psychedelic assisted psychotherapy. Administration of the antagonists in Table 20 and Table 21 allows a new degree of control over the psychedelic experience by controlling both the duration and intensity of the psychedelic experience including controlling, visual hallucinations, auditory hallucinations, sensory hallucinations, depersonalization, delusions, ego loss, elation, feelings of being in other worlds, changes in the perception of time, altered perception of space, changes in the perception of bodily sensations, oceanic boundlessness, connections to higher powers, perception of inner peace, perception of love, altered perception of boundaries between self and surroundings, altered sense of memory, sensations of awe, and sensations of fear, Anxious ego dissolution, Visionary restructuralization, Vigilance reduction, Auditory alterations.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating depression in a human, the method comprising:
(a) administering a psychedelic to the human with depression, and
(b) administering to olanzapine, risperidone, or quetiapine to the human after the administration of the psychedelic in an amount to suppresses or halt hallucinogenic effects of the psychedelic;
wherein the depression is major depressive disorder, persistent depressive disorder, bipolar disorder, treatment resistant depression (TRD), postpartum depression, premenstrual dysphoric disorder, or seasonal affective disorder.

2. The method of claim 1, wherein olanzapine, risperidone, or quetiapine is administered to the human at most about 3 hours after the administration of the psychedelic.

3. The method of claim 1, wherein olanzapine, risperidone, or quetiapine is administered to the human at most about 2 hours after the administration of the psychedelic.

4. The method of claim 1, wherein olanzapine, risperidone, or quetiapine is administered to the human at most about 1 hour after the administration of the psychedelic.

5. The method of claim 1, wherein olanzapine, risperidone, or quetiapine is administered to the human at most about 0.5 hours after the administration of the psychedelic.

6-9. (canceled)

10. The method of claim 1, wherein the psychedelic is an agonist of serotonin receptor 2A.

11. The method of claim 1, wherein the psychedelic is selected from psilocin, psilocybin, N,N-dimethyl-tryptamine (DMT), 4-Acetoxy-DMT, lysergic acid diethylamine (LSD), 1-acetyl LSD (ALD-52), 1P-LSD, 5-MeO-DMT, 2C-B, ibogaine, 3,4-methylenedioxy-methamphetamine (MDMA), DOM, mescaline, (R)-MDMA, (S)-MDMA, 1,3-benzodioxolyl-N-methylbutanamine (MBDB), Methylone, (R)-methylone, (S)-methylone, N-ethyl-3,4-methylenedioxyamphetamine (MDEA), (S)-MDEA, (R)-MDEA, N-ethyl-2-(5-fluoro-1H-indol-3yl)-N-methylethan-1-amine, 4-OH-N,N-Diisopropyltryptamine (4-OH-DiPT) hemi-glutarate, 5,6-dimethoxy-2-aminoindane, 5-methoxy-2-aminoindane, and 2-Br-LSD.

12. The method of claim 11, wherein the psychedelic is psilocybin, lysergic acid diethylamine, N,N-dimethyl-tryptamine, 5-MeO-N,N-dimethyl-tryptamine, 0-acetyl psilocin, or psilocin.

13-17. (canceled)

18. The method of claim 1, wherein the psychedelic is a phenethylamine psychedelic, a tryptamine psychedelic, or an ergot psychedelic; and olanzapine is administered to the human.

19. The method of claim 1, wherein the psychedelic is a phenethylamine psychedelic, a tryptamine psychedelic, or an ergot psychedelic; and risperidone is administered to the human.

20. The method of claim 1, wherein the psychedelic is a phenethylamine psychedelic, a tryptamine psychedelic, or an ergot psychedelic; and quetiapine is administered to the human.

21. The method of claim 1, wherein the psychedelic is administered to the human orally, intravenously, subcutaneously, by inhalation, or by an injection.

22. The method of claim 1, wherein olanzapine, risperidone, or quetiapine is administered to the human orally, intravenously, subcutaneously, by inhalation, or by an injection.

23. A method of reducing adverse effects of a psychedelic used in the treatment of depression in a human, the method comprising:
(a) administering a psychedelic to the human with depression, and
(b) administering olanzapine, risperidone, or quetiapine to the human after the administration of the psychedelic in an amount that reduces adverse effects of the psychedelic experienced by the human,
wherein the depression is major depressive disorder, persistent depressive disorder, bipolar disorder, treatment resistant depression (TRD), postpartum depression, premenstrual dysphoric disorder, or seasonal affective disorder.

24. The method of claim 23, wherein the olanzapine, risperidone, or quetiapine is administered to the human at most about 3 hours after the administration of the psychedelic.

25. The method of claim 23, wherein the psychedelic is selected from the group consisting of psilocin, psilocybin, N,N-dimethyl-tryptamine (DMT), 4-Acetoxy-DMT, lysergic acid diethylamine (LSD), 1-acetyl LSD (ALD-52), 1P-LSD, 5-MeO-DMT, 2C-B, ibogaine, 3,4-methylenedioxy-methamphetamine (MDMA), DOM, mescaline, (R)-MDMA, (S)-MDMA, 1,3-benzodioxolyl-N-methylbutanamine (MBDB), Methylone, (R)-methylone, (S)-methylone, N-ethyl-3,4-methylenedioxyamphetamine (MDEA), (S)-MDEA, (R)-MDEA, N-ethyl-2-(5-fluoro-1H-indol-3yl)-N-methylethan-1-amine, 4-OH-N,N-Diisopropyltryptamine (4-OH-DiPT) hemi-glutarate, 5,6-dimethoxy-2-aminoindane, 5-methoxy-2-aminoindane, and 2-Br-LSD.

26-30. (canceled)

31. A method of treating depression in a human, the method comprising:
(a) administering psilocybin or lysergic acid diethylamine to the human with depression, and
(b) administering to olanzapine, risperidone, or quetiapine to the human after the administration of psilocybin or lysergic acid diethylamine in an amount to suppresses or halt hallucinogenic effects of the psilocybin or lysergic acid diethylamine;
wherein the depression is major depressive disorder, persistent depressive disorder, bipolar disorder, treatment resistant depression (TRD), postpartum depression, premenstrual dysphoric disorder, or seasonal affective disorder.

32. The method of claim 31, wherein the olanzapine, risperidone, or quetiapine is administered to the human at most about 3 hours after the administration of the psilocybin or lysergic acid diethylamine.

33. A method of reducing adverse effects of a psychedelic used in the treatment of a depression in a human, the method comprising:
(a) administering psilocybin or lysergic acid diethylamine to the human with depression, and
(b) administering olanzapine, risperidone, or quetiapine to the human after the administration of the psilocybin or lysergic acid diethylamine in an amount that reduces adverse effects of the psilocybin or lysergic acid diethylamine experienced by the human,
wherein the depression is major depressive disorder, persistent depressive disorder, bipolar disorder, treatment resistant depression (TRD), postpartum depression, premenstrual dysphoric disorder, or seasonal affective disorder; and
wherein adverse effects of the psilocybin or lysergic acid diethylamine comprises hallucinogenic effects, bad trips, psychosis induced by psilocybin or lysergic acid diethylamine, and treating overdose of psilocybin or lysergic acid diethylamine.

34. The method of claim 33, wherein the olanzapine, risperidone, or quetiapine is administered to the human at most about 3 hours after the administration of the psilocybin or lysergic acid diethylamine.
